(19) United States
(12) Patent Application Publication
(10) Pub. No.: US 2026/0022116 A1
(43) Pub. Date: Jan. 22, 2026

(54) IMIDAZOPYRIDINE COMPOUNDS, PREPARATION THEREOF AND THERAPEUTIC USES THEREOF

(71) Applicant: SANOFI, Paris (FR)

(72) Inventors: Maria Esther Arranz Plaza, Paris (FR); Florian Alain Auger, Paris (FR); Patrick Bernardelli, Paris (FR); Guillaume Begis, Paris (FR); Victor Certal, Paris (FR); Ingrid Devillers, Paris (FR); Virginie Rosine Duteil, Paris (FR); Christophe Marcireau, Paris (FR); Franck Slowinski, Paris (FR); Jidong Zhang, Paris (FR)

(21) Appl. No.: 18/995,259

(22) PCT Filed: Jul. 19, 2023
(86) PCT No.: PCT/EP2023/070084

§ 371 Date: Jan. 16, 2025

(30) Foreign Application Priority Data
Jul. 20, 2022 (EP) 22186122.2

(51) Int. Cl. C07D 471/04; A61K 31/437; A61K 31/444; A61K 31/4545; A61K 31/4725; A61K 31/496; A61K 31/498; A61K 31/519; A61K 31/5377; A61K 31/538; A61K 31/5383; A61K 31/5386; A61K 31/541; A61K 31/553; C07D 519/00

(52) U.S. Cl. CPC C07D 471/04; A61K 31/437; A61K 31/444; A61K 31/4545; A61K 31/4725; A61K 31/496; A61K 31/498; A61K 31/519; A61K 31/5377; A61K 31/538; A61K 31/5383; A61K 31/5386; A61K 31/541; A61K 31/553; C07D 519/00

(57) ABSTRACT

Compound are provided which can inhibit ERK5. Also provided are pharmaceutical compositions and medical uses of the same, including the use in treating or preventing conditions such as cancers.

Compounds are provided which can inhibit ERK5. Also provided are pharmaceutical compositions and medical uses of the same, including the use in treating or preventing conditions such as cancers.

### SUMMARY

The mitogen-activated protein kinase (MAPK) cascade is a highly-conserved cellular pathway which transmits signals from the cell surface to the nucleus. The pathway plays an important role in cell proliferation, differentiation, and migration and it is well known to be involved in the development of cancer. Proteins in the pathway include the extracellular signal-regulated kinase (ERK) proteins; among those, ERK5 (expressed from the MAPK7 gene) plays an important role in cell proliferation, as well as epithelial development and neural differentiation (see, e.g., Nishimoto et al., EMBO Reports (2006) 7(8):782-786). ERK5 is unique among the ERK proteins, having a large C-terminal domain which contains a transcriptional activation domain (TAD) as well as a nuclear localization signal and two proline-rich regions (see, e.g., Guo et al., Exp Ther Med. (2020) 19:1997-2007). Autophosphorylation of the TAD is required for transcriptional activation (see, e.g., Morimoto et al., J Biol Chem. (2007) 282(49):35449-35456).

ERK5 plays an important role in controlling cell proliferation and cell cycle progression, for example via direct or indirect phosphorylation of MEF2C, cMYC, SGK1, RSK, FOS, and FRA1 among others (see, e.g., Paudel et al., Int J Mol Sci. (2021) 22:7594-7614; Terasawa et al., Genes to Cells (2003) 8(3):263-273). The involvement of ERK5 in numerous biological pathways means that its activity is associated with many aspects of cancer progression, including tumour angiogenesis, metastasis, inflammation, sustained proliferation, and evasion of growth suppression. It therefore presents an attractive target for modulating disease pathology and treatment in a wide range of conditions. In previous studies, ERK5 inhibition or down-regulation has been shown to block tumorigenesis in murine leukaemia cells, reduce growth of chronic myeloid leukaemia cells, inhibit growth of breast cancer and multiple myeloma cells, suppress colon cancer cell proliferation, and have to have an impact on renal cell carcinoma, mesothelioma, adenocarcinoma, neuroblastoma and hepatocellular carcinoma cell growth or survival, among others (see, e.g., Stecca et al., Int J Mol Sci. (2019) 20:1426-1446).

ERK5 inhibition thus represents a promising approach to tackle a broad range of cancers. Several ERK5 inhibitors have been developed and some are under clinical review. For example, WO 2019/170543 (Bayer AG and Bayer Pharma AG) discloses compounds which are said to be active as ERK5 inhibitors in the μM to nM concentration range. In particular, ERK5 inhibition has been reported to have potential use in the treatment of melanoma in combination with BRAF-MEK1/2-ERK1/2 pathway inhibitors to overcome intrinsic resistance and hinder or delay some modes of acquired resistance (see, e.g., Cook et al., Front. Cell Dev. Biol. (2022)).

Despite recent progress in cancer treatment with the development of targeted therapies and immunotherapies, not all cancer patients can be offered an efficient therapeutic solution.

There is therefore a need to identify and develop new drugs. The present disclosure seeks to address this need by providing novel compounds for use as ERK5 inhibitors and for the treatment of ERK5 related diseases and conditions.

Accordingly, a first aspect provides a compound, being of Formula (I)

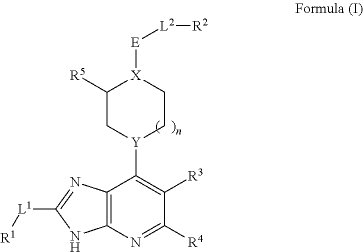

or a pharmaceutically acceptable salt thereof, wherein:
   R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
      wherein each R^{A} is independently selected from halo, —OH, —NHR′, —CN, oxo, —SO₂CH₃, —(C₁-C₃)alkyl, —O(C₁-C₃)alkyl, —C(O)R″, —C(O)NHR′, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, —NH₂, and —CN, and/or wherein two occurrences of R^{A} may be taken together with the atom(s) to which they are attached to form a (C₃-C₆)cycloalkyl group, or a 3- to 6-membered heterocycloalkyl group,
      wherein each R′ is independently selected from —H, methyl, and phenyl, and
      wherein each R″ is independently selected from —OH, methyl, phenyl optionally substituted by —OCF₃, and tetrahydrofuranyl;
   L¹ is selected from a direct bond, —O—, —CH₂—, and —CH═;
   R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
      wherein each R^{B} is selected from halo, —OH, oxo, —NH₂, —NHMe, —NO₂, —CN, —SF₅, —SiMe₃, —B(OH)₂, —(C₁-C₃)alkyl, —O(C₁-C₃)alkyl, —(C₃-C₆)cycloalkyl, —SO₂CH₃, —NHC(O)Me, phenyl, benzyl, 1-λ⁶-2-thiazolidine-1,1-dionyl, and pyridyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
      and further wherein, when R² is —(C₃-C₆)cycloalkyl, R² is optionally substituted at one ring atom by a spirocyclic oxindolyl group;
   L² is selected from a direct bond, —CH₂—, —CH₂CH₂—, and —CH₂O—, wherein L², when present, is optionally substituted with —NH₂ or methyl;
   R³ is selected from —H, halo, —OH, and methyl optionally substituted with —OH;
   R⁴ is —H, or —NH₂;
   R⁵ is —H, or methyl;
   X is CH or N;
   Y is selected from CH, CF, and N (with the proviso that when Y is N, X is also N);
   E is selected from —C(O)—, —S(O)₂—, and —O— (with the proviso that when E is —O—, X and Y are both CH); and
n is 0, 1, or 2.

In embodiments, the compound is a compound of Formula (I-A)

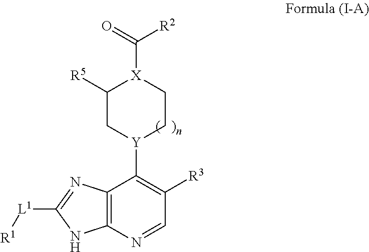

or a pharmaceutically acceptable salt thereof, wherein R¹, R², R³, R⁵, L¹, X, Y, and n are as defined hereinbefore.

In embodiments, the compound is a compound of Formula (I-B)

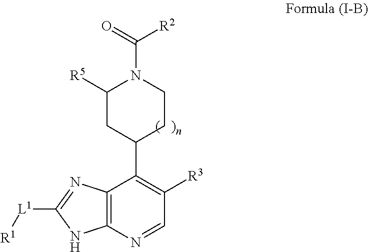

or a pharmaceutically acceptable salt thereof, wherein R¹, R², R³, R⁵, L¹, and n are as defined hereinbefore.

In embodiments, the compound is as defined hereinbefore, wherein:
R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
   wherein each R^{A} is independently selected from halo, —OH, —NHR′, —CN, oxo, —SO₂CH₃, —(C₁-C₃)alkyl, —O(C₁-C₃)alkyl, —C(O)R″, —C(O)NHR′, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, —NH₂, and —CN,
   and/or wherein two occurrences of R^{A} may be taken together with the atom(s) to which they are attached to form a (C₃-C₆)cycloalkyl group, or a 3- to 6-membered heterocycloalkyl group,
   wherein each R′ is independently selected from —H, methyl, and phenyl, and
   wherein each R″ is independently selected from methyl, phenyl optionally substituted by —OCF₃, and tetrahydrofuranyl;
R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
   wherein each R^{B} is selected from halo, —OH, —NH₂, —NO₂, —CN, —SF₅, —B(OH)₂, —(C₁-C₃)alkyl, —O(C₁-C₃)alkyl, —(C₃-C₆)cycloalkyl, —SO₂CH₃, phenyl, benzyl, 1-λ⁶-2-thiazolidine-1,1-dionyl, and pyridyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
   and further wherein, when R² is —(C₃-C₆)cycloalkyl, R² is optionally substituted at one ring atom by a spirocyclic oxindolyl group; and
X and Y are each independently selected from CH, and N (with the proviso that when Y is N, X is also N).

In embodiments, the compound is a compound of Formula (I-C) or Formula (I-D)

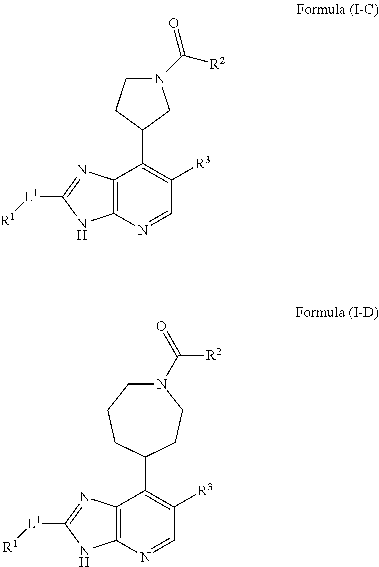

or a pharmaceutically acceptable salt thereof, wherein R¹, R², R³, and L¹ are as defined hereinbefore.

In embodiments, R² is selected from —(C₃-C₆)cycloalkyl, 5- to 6-membered heterocycloalkyl, 9- to 10-membered heterocycloalkyl, —(C₆-C₁₀)aryl, 5- to 6-membered heteroaryl, and 9- to 10-membered heteroaryl, wherein R² is optionally substituted with one or more occurrences of R^{B} as defined hereinbefore.

In embodiments, R² is selected from:

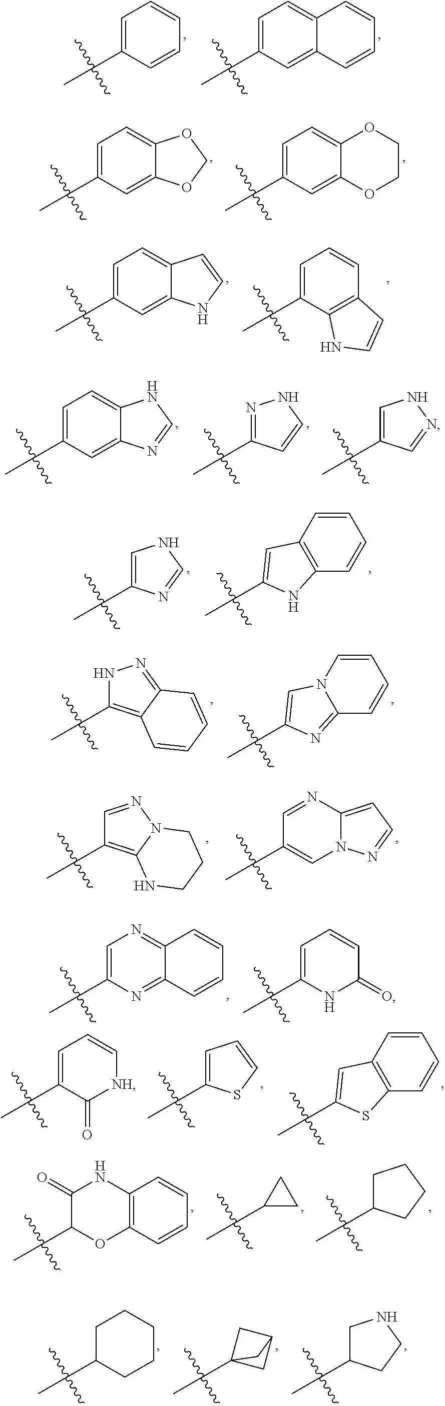

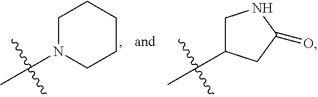

wherein R² is optionally substituted by one or more occurrences of R^{B} as defined hereinbefore.

In embodiments, R² is selected from:

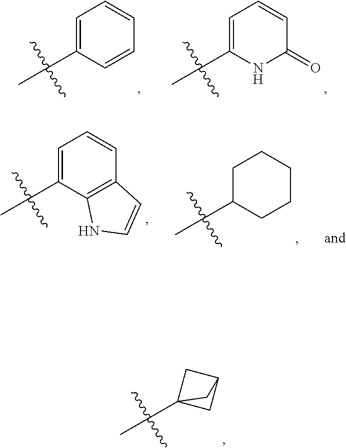

wherein R² is optionally substituted by one or more occurrences of R^{B}, wherein each R^{B} is independently selected from the group consisting of halo, —NH₂, —O(C₁-C₃)alkyl, —(C₁-C₃)alkyl, —(C₃-C₆)cycloalkyl, —SF₅, and —NO₂, and wherein each occurrence of —O(C₁-C₃)alkyl, —(C₁-C₃)alkyl, and —(C₃-C₆)cycloalkyl, is optionally substituted by one or more groups independently selected from halo and —CF₃.

In embodiments, at least one occurrence of R^{B} is —SF₅.

In embodiments, R² is:

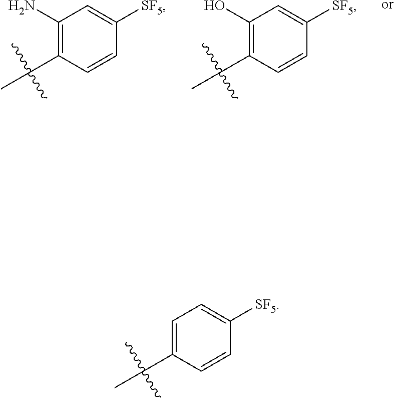

In embodiments, R¹ is selected from —H, —(C₁-C₃)alkyl, —(C₃-C₆)cycloalkyl, —(C₅-C₇)cycloalkenyl, —(C₆-C₁₀)aryl, 4- to 9-membered heterocycloalkyl, 6-membered heterocycloalkenyl, 5- to 6-membered heteroaryl, and 9- to 10-membered heteroaryl, wherein R¹ is optionally substituted with one or more occurrences of R^{A} as defined hereinbefore.

In embodiments, R¹ is selected from:

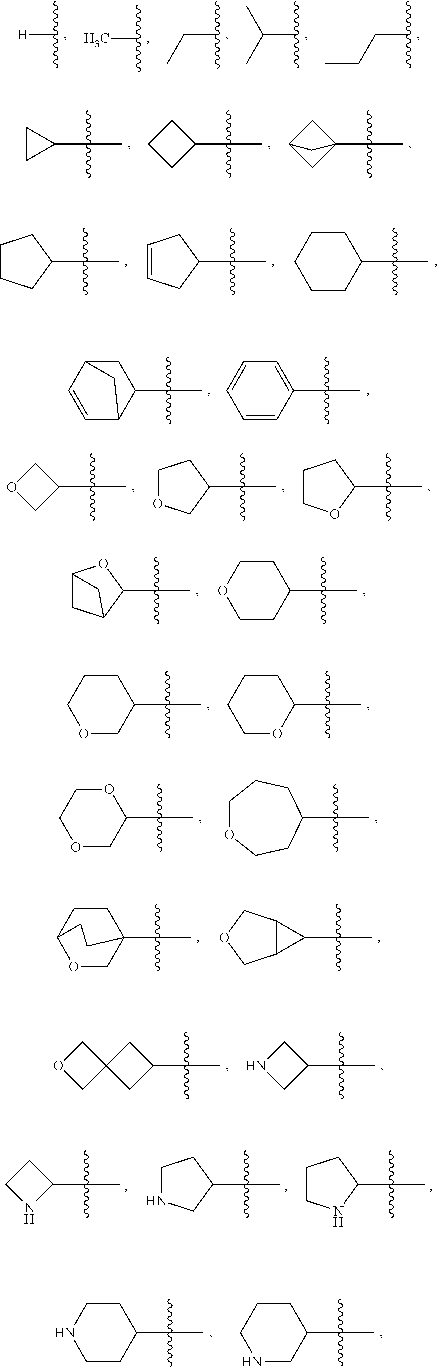

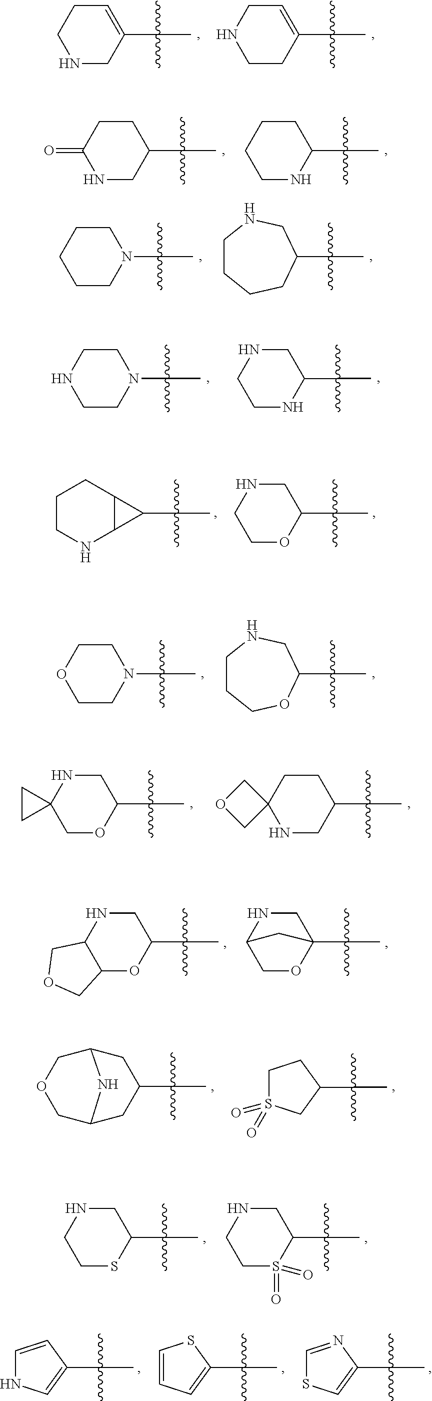

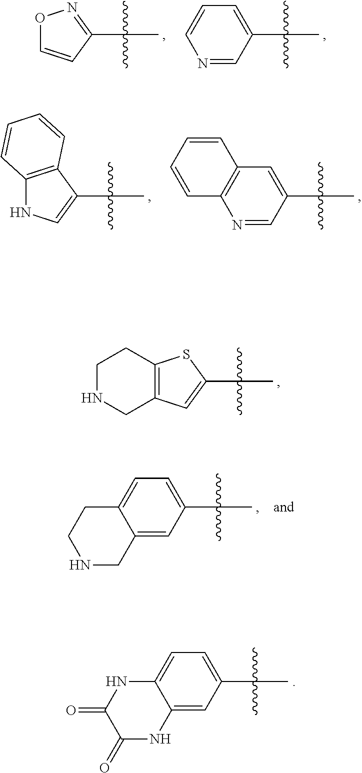

wherein R¹ is optionally substituted by one or more occurrences of R^{A} as defined hereinbefore.

In embodiments, R¹ is selected from:

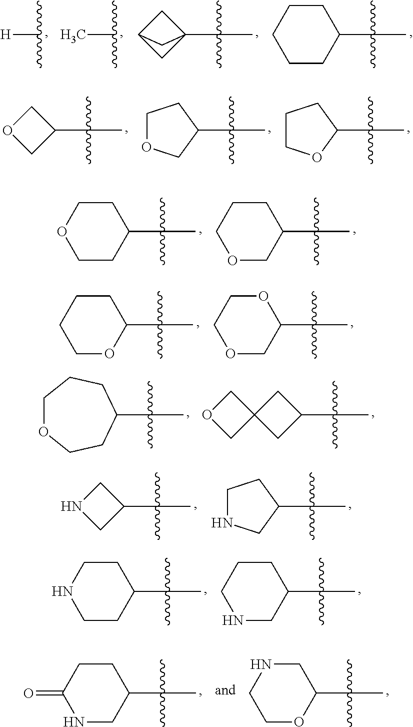

wherein R¹ is optionally substituted by one or more occurrences of R^{A}, wherein each R^{A} is independently selected from the group consisting of -halo, —OH, —NH₂, oxo, —(C₁-C₃)alkyl, —C(O)R″, and —O(C₁-C₃)alkyl, wherein each occurrence of (C₁-C₃)alkyl may be optionally substituted by one or more halo groups, and wherein each R″ is independently selected from methyl, phenyl optionally substituted by —OCF₃, and tetrahydrofuranyl.

In embodiments, R¹ is selected from —(C₄-C₈)cycloalkenyl, —(C₆-C₁₀)aryl, 7- to 10-membered multicyclic (e.g., bicyclic) heterocycloalkyl, 5- to 8-membered heterocycloalkenyl, and 7- to 10-membered multicyclic (e.g., bicyclic) heteroaryl, wherein R¹ is optionally substituted with one or more occurrences of R^{A} as defined hereinbefore, or R¹ is —(C₁-C₆)alkyl substituted by one or more substituents selected from —OH, —NH₂, and —O(C₁-C₃)alkyl.

In embodiments, L¹ is a direct bond or —CH₂—.

Another aspect provides a compound, being of Formula (II)

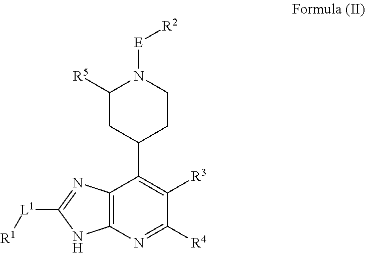

or a pharmaceutically acceptable salt thereof, wherein R¹, L¹, R³, R⁴, and R⁵ are as defined hereinbefore, and wherein:
   R² is selected from —(C₃-C₆)cycloalkyl (e.g., cyclopropyl), —(C₆-C₁₀)aryl, 5- to 10-membered heterocycloalkyl, and 5- to 10-membered heteroaryl, wherein R² is optionally substituted by one or more occurrences of R^{B}, wherein each R^{B} is independently selected from the group consisting of halo, —OH, oxo, —NO₂, —NH₂, —NHMe, —CN, —SF₅, —SO₂CH₃, —NHC(O)Me, —SiMe₃, —(C₁-C₃)alkyl optionally substituted by one or more halo, —O(C₁-C₃)alkyl optionally substituted by one or more halo, —(C₃-C₆)cycloalkyl optionally substituted by —CF₃, phenyl optionally substituted by one or more halo, —OH, or —CF₃, benzyl optionally substituted by halo, and 1-λ⁶-2-thiazolidine-1,1-dionyl,
      and further wherein, when R² is cyclopropyl, R² is optionally substituted at one ring atom by a spirocyclic oxindolyl group; and
   E is —C(O)— or —S(O)₂—.

A further aspect provides a compound, being of Formula (III)

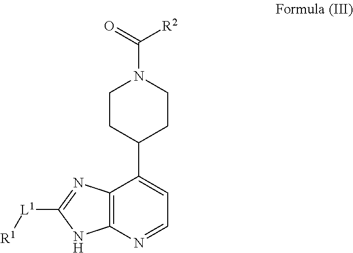

or a pharmaceutically acceptable salt thereof, wherein:
   R¹ is —H, —(C₁-C₃)alkyl, —(C₄-C₆)cycloalkyl, —(C₅-C₇)cycloalkenyl, —(C₆-C₁₀)aryl, or 4- to 9-membered heterocycloalkyl, 6-membered heterocycloalkenyl, 5- to 6-membered heteroaryl, and 9- to 10-membered heteroaryl, wherein R¹ is optionally substituted by one or more R^{A}, wherein each R^{A} is independently selected from halo, —OH, oxo, —NH₂, —NHMe, —C(O)OH, —C(O)Me, —SO₂CH₃, —NHC(O)R″, —(C₁-C₃)alkyl, —O(C₁-C₃)alkyl, cyclopropyl, oxetanyl, sulfolanyl, phenyl, benzyl, and pyridyl, wherein each occurrence of —(C₁-C₃)alkyl, —O(C₁-C₃)alkyl, and phenyl is optionally substituted by one or more groups independently selected from halo, —OH, and —NH₂,
      wherein each R″ is independently selected from methyl, and tetrahydrofuranyl;
   L¹ is a direct bond or —CH₂—; and
   R² is selected from —(C₃-C₆)cycloalkyl, —(C₆-C₁₀)aryl, 5- to 6-membered heterocycloalkyl, and 5- to 10-membered heteroaryl, wherein R² is optionally substituted by one or more occurrences of R^{B}, wherein each R^{B} is independently selected from the group consisting of halo, —NO₂, —NH₂, —NHMe, —NHAc, —O(C₁-C₃)alkyl, —SF₅, —SiMe₃, —(C₁-C₃)alkyl, 1-λ⁶-2-thiazolidine-1,1-dionyl, phenyl, benzyl, and —(C₃-C₆)cycloalkyl optionally substituted by —CF₃, wherein each occurrence of —(C₁-C₃)alkyl, —O(C₁-C₃)alkyl, phenyl, and benzyl, is optionally substituted by one or more groups independently selected from halo, —NH₂, and —CF₃, and further wherein, when R² is cyclopropyl, R² is optionally substituted at one ring atom by a spirocyclic oxindolyl group.

A further aspect provides a compound, being of Formula (IV)

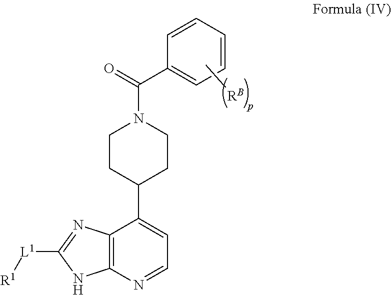

or a pharmaceutically acceptable salt thereof, wherein R¹, and L¹ are as defined hereinbefore, and wherein:
   p is 1, 2, 3, 4, or 5; and
   each R^{B} is independently selected from the group consisting of halo, —NO₂, —NH₂, —NHMe, —NHAc, —OCH₃, —OCF₃, —SF₅, SiMe₃, (C₁-C₃)alkyl, —O(C₁-C₃)alkyl, 1-λ⁶-2-thiazolidine-1,1-dionyl, phenyl, and —(C₃-C₆)cycloalkyl optionally substituted by —CF₃, wherein each occurrence of —(C₁-C₃)alkyl, —O(C₁-C₃)alkyl, and phenyl is optionally substituted by one or more groups independently selected from halo, —NH₂, and —CF₃.

A further aspect provides a compound, being of Formula (V)

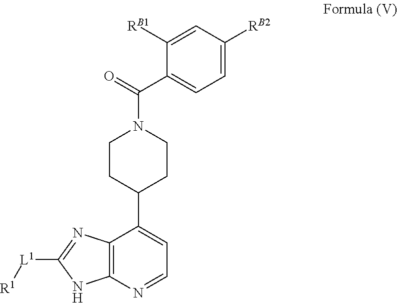

or a pharmaceutically acceptable salt thereof, wherein R¹, and L¹ are as defined hereinbefore, and wherein:
   R^{B1} is either —H, or is selected from the group consisting of halo (e.g. —F, or —Cl), —OH, —NH₂, —NHMe, —NHAc, and —NO₂; and
   R^{B2} is selected from the group consisting of halo (e.g. —Br), —SF₅, —SO₂CH₃, —SiMe₃, —(C₁-C₃)alkyl optionally substituted by one or more halo groups, —O(C₁-C₃)alkyl optionally substituted by one or more halo groups, 1-trifluoromethylcyclopropan-1-yl, and phenyl optionally substituted by one or more groups independently selected from halo, or —NH₂.

In embodiments, R^{B2} is —SF₅.

A further aspect provides a compound, being of Formula (VIII), (IX), or (X)

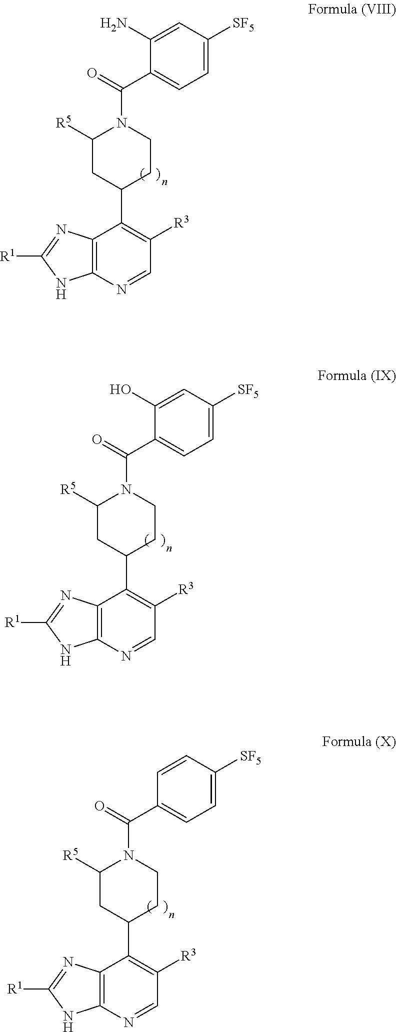

or a pharmaceutically acceptable salt thereof, wherein R¹, R³, R⁵, and n are as defined hereinbefore.

A further aspect provides a compound selected from the group consisting of:
[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
[2-amino-4-(trifluoromethoxy)phenyl]-[4-(2-tetrahydrofuran-3-yl-3H-imidazo[4,5-b]pyridin-7-yl)-1-piperidyl]methanone,
[2-amino-4-(trifluoromethoxy)phenyl]-[4-(2-tetrahydropyran-4-yl-3H-imidazo[4,5-b]pyridin-7-yl)-1-piperidyl]methanone,
[4-[2-(4-piperidyl)-3H-imidazo[4,5-b]pyridin-7-yl]-1-piperidyl]-[4-(trifluoromethoxy)phenyl]methanone,
trans-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-aminocyclohexyl)-3H-imidazo[4,5-b]pyridin-7-yl]-1-piperidyl]-[4-(trifluoromethoxy)phenyl]methanone,
1-[3-[7-[1-[4-(trifluoromethoxy)benzoyl]-4-piperidyl]-3H-imidazo[4,5-b]pyridin-2-yl]azetidin-1-yl]ethanone,
[4-(2-tetrahydropyran-4-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(1-methyl-4-piperidyl)-3H-imidazo[4,5-b]pyridin-7-yl]-1-piperidyl]methanone,
[4-[2-(1-methyl-4-piperidyl)-3H-imidazo[4,5-b]pyridin-7-yl]-1-piperidyl]-[4-(trifluoromethoxy)phenyl]methanone,
[4-(2-methyl-3H-imidazo[4,5-b]pyridin-7-yl)-1-piperidyl]-[4-(trifluoromethoxy)phenyl]methanone,
[4-[2-(3-methoxy-1-bicyclo[1.1.1]pentanyl)-3H-imidazo[4,5-b]pyridin-7-yl]-1-piperidyl]-[4-(trifluoromethoxy)phenyl]methanone,
5-[7-[1-[4-(trifluoromethoxy)benzoyl]-4-piperidyl]-3H-imidazo[4,5-b]pyridin-2-yl]piperidin-2-one,
[4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone,
[4-(3H-imidazo[4,5-b]pyridin-7-yl)-1-piperidyl]-[4-(trifluoromethoxy)phenyl]methanone,
[4-[2-(oxetan-3-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone,
[4-[2-(azetidin-3-yl)-3H-imidazo[4,5-b]pyridin-7-yl]-1-piperidyl]-[4-(trifluoromethoxy)phenyl]methanone,
[4-(2-morpholin-2-yl-3H-imidazo[4,5-b]pyridin-7-yl]-1-piperidyl]-[4-(trifluoromethoxy)phenyl]methanone,
[4-[2-(1-methylpyrrolidin-3-yl)-3H-imidazo[4,5-b]pyridin-7-yl]-1-piperidyl]-[4-(trifluoromethoxy)phenyl]methanone,
[4-(2-tetrahydrofuran-3-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone,
[2-amino-4-(trifluoromethoxy)phenyl]-[4-(2-morpholin-2-yl-3H-imidazo[4,5-b]pyridin-7-yl)-1-piperidyl]methanone,
[4-[2-(2,2-dimethyltetrahydropyran-4-yl)-3H-imidazo[4,5-b]pyridin-7-yl]-1-piperidyl]-[4-(trifluoromethoxy)phenyl]methanone,
[4-[2-(6,6-dimethyltetrahydropyran-3-yl)-3H-imidazo[4,5-b]pyridin-7-yl]-1-piperidyl]-[4-(trifluoromethoxy)phenyl]methanone,
[4-[2-(1,4-dioxan-2-yl)-3H-imidazo[4,5-b]pyridin-7-yl]-1-piperidyl]-[4-(trifluoromethoxy)phenyl]methanone,
[4-(2-tetrahydrofuran-2-yl-3H-imidazo[4,5-b]pyridin-7-yl)-1-piperidyl]-[4-(trifluoromethoxy)phenyl]methanone,
[4-[2-(4-methylmorpholin-2-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(5,5-difluoro-3-piperidyl)-3H-imidazo[4,5-b]pyridin-7-yl]-1-piperidyl]methanone,
[2-amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone,
[4-[2-[1-(2,2-difluoroethyl)azetidin-3-yl]-3H-imidazo[4,5-b]pyridin-7-yl]-1-piperidyl]-[4-(trifluoromethoxy)phenyl]methanone,
[4-[2-(oxetan-3-ylmethyl)-3H-imidazo[4,5-b]pyridin-7-yl]-1-piperidyl]-[4-(trifluoromethoxy)phenyl]methanone,
(4-chloro-1H-indol-7-yl)-[4-(2-tetrahydropyran-4-yl-3H-imidazo[4,5-b]pyridin-7-yl)-1-piperidyl]methanone,
[2-nitro-4-(pentafluoro-λ⁶-sulfanyl)phenyl]-[4-(2-tetrahydropyran-4-yl-3H-imidazo[4,5-b]pyridin-7-yl)-1-piperidyl]methanone,
[4-[2-(tetrahydrofuran-3-ylmethyl)-3H-imidazo[4,5-b]pyridin-7-yl]-1-piperidyl]-[4-(trifluoromethoxy)phenyl]methanone,
[4-[2-(oxepan-4-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(oxepan-4-yl)-3H-imidazo[4,5-b]pyridin-7-yl]-1-piperidyl]methanone,
[4-[2-(2-oxaspiro[3.3]heptan-6-ylmethyl)-3H-imidazo[4,5-b]pyridin-7-yl]-1-piperidyl]-[4-(trifluoromethoxy)phenyl]methanone,
[4-(1,1,2,2,2-pentafluoroethyl)phenyl]-[4-(2-tetrahydropyran-4-yl-3H-imidazo[4,5-b]pyridin-7-yl)-1-piperidyl]methanone,
[4-(pentafluoro-6-sulfanyl)phenyl]-[4-(2-tetrahydropyran-4-yl-3H-imidazo[4,5-b]pyridin-7-yl)-1-piperidyl]methanone,
(cis)-[4-(2-tetrahydropyran-4-yl-3H-imidazo[4,5-b]pyridin-7-yl)-1-piperidyl]-[4-(2,2,2-trifluoroethyl)cyclohexyl]methanone,
(trans)-[4-(2-tetrahydropyran-4-yl-3H-imidazo[4,5-b]pyridin-7-yl)-1-piperidyl]-[4-(2,2,2-trifluoroethyl)cyclohexyl]methanone,
[2-amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone,
[2-amino-4-(trifluoromethoxy)phenyl]-[4-[2-(oxetan-3-ylmethyl)-3H-imidazo[4,5-b]pyridin-7-yl]-1-piperidyl]methanone,
(4-methoxy-1H-indol-7-yl)-[4-(2-tetrahydropyran-4-yl-3H-imidazo[4,5-b]pyridin-7-yl)-1-piperidyl]methanone,
6-[4-(2-tetrahydropyran-4-yl-3H-imidazo[4,5-b]pyridin-7-yl)piperidine-1-carbonyl]-3-(trifluoromethyl)-1H-pyridin-2-one,
[4-(2-tetrahydropyran-4-yl-3H-imidazo[4,5-b]pyridin-7-yl)-1-piperidyl]-[4-(2,2,2-trifluoroethyl)phenyl]methanone,
[4-(trifluoromethoxy)phenyl]-[4-[2-(trifluoromethyl)-3H-imidazo[4,5-b]pyridin-7-yl]-1-piperidyl]methanone,
[4-(2-tetrahydropyran-2-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-(2-tetrahydropyran-2-yl-3H-imidazo[4,5-b]pyridin-7-yl)-1-piperidyl]methanone,
(3-methoxy-1-bicyclo[1.1.1]pentanyl)-[4-(2-tetrahydropyran-4-yl-3H-imidazo[4,5-b]pyridin-7-yl)-1-piperidyl]methanone,
[4-(2-tetrahydropyran-4-yl-3H-imidazo[4,5-b]pyridin-7-yl)-1-piperidyl]-[3-(trifluoromethyl)-1-bicyclo[1.1.1]pentanyl]methanone,
[4-(2-tetrahydropyran-4-yl-3H-imidazo[4,5-b]pyridin-7-yl)-1-piperidyl]-[4-[1-(trifluoromethyl)cyclopropyl]phenyl]methanone,
[4-[2-(5,5-difluoro-3-piperidyl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(tetrahydropyran-4-ylmethyl)-3H-imidazo[4,5-b]pyridin-7-yl]-1-piperidyl]methanone,
[4-(2-pyrrolidin-3-yl-3H-imidazo[4,5-b]pyridin-7-yl)-1-piperidyl]-[4-(trifluoromethoxy)phenyl]methanone,
[4-[2-[(1-methyl-4-piperidyl)methyl]-3H-imidazo[4,5-b]pyridin-7-yl]-1-piperidyl]-[4-(trifluoromethoxy)phenyl]methanone,
[2-amino-4-(trifluoromethoxy)phenyl]-[4-(2-tetrahydropyran-3-yl-3H-imidazo[4,5-b]pyridin-7-yl)-1-piperidyl]methanone,
[4-[2-(tetrahydropyran-4-ylmethyl)-3H-imidazo[4,5-b]pyridin-7-yl]-1-piperidyl]-[4-(trifluoromethoxy)phenyl]methanone,
[4-(2-tetrahydropyran-3-yl-3H-imidazo[4,5-b]pyridin-7-yl)-1-piperidyl]-[4-(trifluoromethoxy)phenyl]methanone, and
[4-[2-[1-(2,2-difluoroethyl)-4-piperidyl]-3H-imidazo[4,5-b]pyridin-7-yl]-1-piperidyl]-[4-(trifluoromethoxy)phenyl]methanone,
   and the pharmaceutically acceptable salts thereof.

A further aspect provides a compound selected from the group consisting of:
[2-amino-4-(trifluoromethoxy)phenyl]-[4-[2-(oxetan-3-yl)-3H-imidazo[4,5-b]pyridin-7-yl]-1-piperidyl]methanone;
[4-[2-[tetrahydrofuran-2-yl]-3H-imidazo[4,5-b]pyridin-7-yl]-1-piperidyl]-[4-(trifluoromethoxy)phenyl]methanone;
[4-[2-(3-hydroxy-1-bicyclo[1.1.1]pentanyl)-3H-imidazo[4,5-b]pyridin-7-yl]-1-piperidyl]-[4-(trifluoromethoxy)phenyl]methanone;
[4-[2-[pyrrolidin-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[tetrahydropyran-2-yl]-3H-imidazo[4,5-b]pyridin-7-yl]-1-piperidyl]methanone;
2-naphthyl-[4-(2-tetrahydropyran-4-yl-3H-imidazo[4,5-b]pyridin-7-yl)-1-piperidyl]methanone;
1H-indazol-3-yl-[4-(2-tetrahydropyran-4-yl-3H-imidazo[4,5-b]pyridin-7-yl)-1-piperidyl]methanone;
[4-(2-tetrahydropyran-4-yl-3H-imidazo[4,5-b]pyridin-7-yl)-1-piperidyl]-[7-(trifluoromethyl)-4,5,6,7-tetrahydropyrazolo[1,5-a]pyrimidin-3-yl]methanone;
(1-methyl-5-phenyl-pyrazol-3-yl)-[4-(2-tetrahydropyran-4-yl-3H-imidazo[4,5-b]pyridin-7-yl)-1-piperidyl]methanone;
(3-amino-2-naphthyl)-[4-(2-tetrahydropyran-4-yl-3H-imidazo[4,5-b]pyridin-7-yl)-1-piperidyl]methanone;
(2-amino-3,4,5,6-tetrafluoro-phenyl)-[4-(2-tetrahydropyran-4-yl-3H-imidazo[4,5-b]pyridin-7-yl)-1-piperidyl]methanone;
[1-isopropyl-2-(trifluoromethyl)benzimidazol-5-yl]-[4-(2-tetrahydropyran-4-yl-3H-imidazo[4,5-b]pyridin-7-yl)-1-piperidyl]methanone;
(2-amino-4-methylsulfonyl-phenyl)-[4-(2-tetrahydropyran-4-yl-3H-imidazo[4,5-b]pyridin-7-yl)-1-piperidyl]methanone;
[3-(1,1-dioxo-1,2-thiazolidin-2-yl)phenyl]-[4-(2-tetrahydropyran-4-yl-3H-imidazo[4,5-b]pyridin-7-yl)-1-piperidyl]methanone;
[2-(3-hydroxyphenyl)cyclopropyl]-[4-(2-tetrahydropyran-4-yl-3H-imidazo[4,5-b]pyridin-7-yl)-1-piperidyl]methanone;
2-[2-chloro-4-(trifluoromethyl)phenoxy]-1-[4-(2-tetrahydropyran-4-yl-3H-imidazo[4,5-b]pyridin-7-yl)-1-piperidyl]propan-1-one;
(3-phenylcyclopentyl)-[4-(2-tetrahydropyran-4-yl-3H-imidazo[4,5-b]pyridin-7-yl)-1-piperidyl]methanone;
[4-hydroxy-1-[2-(trifluoromethyl)phenyl]pyrazol-3-yl]-[4-(2-tetrahydropyran-4-yl-3H-imidazo[4,5-b]pyridin-7-yl)-1-piperidyl]methanone;
[4-[2-[5,5-difluoro-3-piperidyl]-3H-imidazo[4,5-b]pyridin-7-yl]-1-piperidyl]-[4-(trifluoromethoxy)phenyl]methanone;
[3-amino-4-phenyl-5-(trifluoromethyl)-2-thienyl]-[4-(2-tetrahydropyran-4-yl-3H-imidazo[4,5-b]pyridin-7-yl)-1-piperidyl]methanone;
(7-hydroxy-1H-indol-2-yl)-[4-(2-tetrahydropyran-4-yl-3H-imidazo[4,5-b]pyridin-7-yl)-1-piperidyl]methanone;
[4-[2-(1-cyclopropyl-4-piperidyl)-3H-imidazo[4,5-b]pyridin-7-yl]-1-piperidyl]-[4-(trifluoromethoxy)phenyl]methanone;
1-[4-[7-[1-[4-(trifluoromethoxy)benzoyl]-4-piperidyl]-3H-imidazo[4,5-b]pyridin-2-yl]-1-piperidyl]ethanone;
[2-amino-4-(pentafluoro-λ⁶-sulfanyl)phenyl]-[4-(2-tetrahydropyran-4-yl-3H-imidazo[4,5-b]pyridin-7-yl)-1-piperidyl]methanone;
(cis)-[4-(2-tetrahydropyran-4-yl-3H-imidazo[4,5-b]pyridin-7-yl)-1-piperidyl]-[4-(trifluoromethoxy)cyclohexyl]methanone;
1-[4-[7-[1-[2-amino-4-(trifluoromethoxy)benzoyl]-4-piperidyl]-3H-imidazo[4,5-b]pyridin-2-yl]-1-piperidyl]ethanone;
[2-amino-4-(trifluoromethoxy)phenyl]-[4-[2-(1-cyclopropyl-4-piperidyl)-3H-imidazo[4,5-b]pyridin-7-yl]-1-piperidyl]methanone;
[4-(2-cyclohexyl-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-(1,4-dioxan-2-yl)-3H-imidazo[4,5-b]pyridin-7-yl]-1-piperidyl]methanone;
2-[4-(2-tetrahydropyran-4-yl-3H-imidazo[4,5-b]pyridin-7-yl)piperidine-1-carbonyl]benzothiophene-5-carbonitrile;
(5-amino-1-phenyl-pyrazol-4-yl)-[4-(2-tetrahydropyran-4-yl-3H-imidazo[4,5-b]pyridin-7-yl)-1-piperidyl]methanone;
2-[2-oxo-2-[4-(2-tetrahydropyran-4-yl-3H-imidazo[4,5-b]pyridin-7-yl)-1-piperidyl]ethyl]-4H-1,4-benzoxazin-3-one;
1-(3,4-difluorophenyl)-4-[4-(2-tetrahydropyran-4-yl-3H-imidazo[4,5-b]pyridin-7-yl)piperidine-1-carbonyl]pyrrolidin-2-one;
[4-(3-chloro-4-fluoro-phenyl)phenyl]-[4-(2-tetrahydropyran-4-yl-3H-imidazo[4,5-b]pyridin-7-yl)-1-piperidyl]methanone;
(7-amino-2-methyl-pyrazolo[1,5-a]pyrimidin-6-yl)-[4-(2-tetrahydropyran-4-yl-3H-imidazo[4,5-b]pyridin-7-yl)-1-piperidyl]methanone;
1-[(2-chlorophenyl)methyl]-4-[4-(2-tetrahydropyran-4-yl-3H-imidazo[4,5-b]pyridin-7-yl)piperidine-1-carbonyl]pyrrolidin-2-one;
1-(4-fluorophenyl)-4-[4-(2-tetrahydropyran-4-yl-3H-imidazo[4,5-b]pyridin-7-yl)piperidine-1-carbonyl]pyrrolidin-2-one;
[1-[(2-chlorophenyl)methyl]pyrazol-4-yl]-[4-(2-tetrahydropyran-4-yl-3H-imidazo[4,5-b]pyridin-7-yl)-1-piperidyl]methanone;
[4-(2-aminophenyl)phenyl]-[4-(2-tetrahydropyran-4-yl-3H-imidazo[4,5-b]pyridin-7-yl)-1-piperidyl]methanone;
(3,6-dichloroimidazo[1,2-a]pyridin-2-yl)-[4-(2-tetrahydropyran-4-yl-3H-imidazo[4,5-b]pyridin-7-yl)-1-piperidyl]methanone;
(4-amino-1-ethyl-pyrazol-3-yl)-[4-(2-tetrahydropyran-4-yl-3H-imidazo[4,5-b]pyridin-7-yl)-1-piperidyl]methanone;
(3-aminoquinoxalin-2-yl)-[4-(2-tetrahydropyran-4-yl-3H-imidazo[4,5-b]pyridin-7-yl)-1-piperidyl]methanone;
2-[4-(2-tetrahydropyran-4-yl-3H-imidazo[4,5-b]pyridin-7-yl)piperidine-1-carbonyl]spiro[cyclopropane-1,3′-indoline]-2′-one;
[1-(2-chlorophenyl)pyrrolidin-3-yl]-[4-(2-tetrahydropyran-4-yl-3H-imidazo[4,5-b]pyridin-7-yl)-1-piperidyl]methanone;
(2-amino-4,5-dichloro-phenyl)-[4-(2-tetrahydropyran-4-yl-3H-imidazo[4,5-b]pyridin-7-yl)-1-piperidyl]methanone;
[1-(4-fluorophenyl)imidazol-4-yl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-(1-methoxy-1-methyl-ethyl)-3H-imidazo[4,5-b]pyridin-7-yl]-1-piperidyl]methanone;
[2-amino-4-(trifluoromethoxy)phenyl]-[4-[2-(1-hydroxy-1-methyl-ethyl)-3H-imidazo[4,5-b]pyridin-7-yl]-1-piperidyl]methanone;
[2-amino-4-(trifluoromethoxy)phenyl]-[4-(2-cyclohexyl-3H-imidazo[4,5-b]pyridin-7-yl)-1-piperidyl]methanone;
[2-amino-4-(trifluoromethoxy)phenyl]-[4-[2-(3-methoxypropyl)-3H-imidazo[4,5-b]pyridin-7-yl]-1-piperidyl]methanone;
[4-[2-(3-amino-1-bicyclo[1.1.1]pentanyl)-3H-imidazo[4,5-b]pyridin-7-yl]-1-piperidyl]-[4-(trifluoromethoxy)phenyl]methanone;
[4-[2-(3-amino-1-bicyclo[1.1.1]pentanyl)-3H-imidazo[4,5-b]pyridin-7-yl]-1-piperidyl]-[2-amino-4-(trifluoromethoxy)phenyl]methanone;
(trans)-[4-(2-tetrahydropyran-4-yl-3H-imidazo[4,5-b]pyridin-7-yl)-1-piperidyl]-[4-(trifluoromethoxy)cyclohexyl]methanone;
[3-fluoro-4-(trifluoromethoxy)phenyl]-[4-(2-tetrahydropyran-4-yl-3H-imidazo[4,5-b]pyridin-7-yl)-1-piperidyl]methanone;
(cis)-[2-amino-4-(trifluoromethoxy)phenyl]-[4-[2-(3-methoxycyclobutyl)-3H-imidazo[4,5-b]pyridin-7-yl]-1-piperidyl]methanone;
(trans)-[2-amino-4-(trifluoromethoxy)phenyl]-[4-[2-(3-methoxycyclobutyl)-3H-imidazo[4,5-b]pyridin-7-yl]-1-piperidyl]methanone;
[4-[2-[(4-methylpiperazin-1-yl)methyl]-3H-imidazo[4,5-b]pyridin-7-yl]-1-piperidyl]-[4-(trifluoromethoxy)phenyl]methanone;
[4-[2-[2-(5-chloro-2-hydroxy-phenyl)thiazol-4-yl]-3H-imidazo[4,5-b]pyridin-7-yl]-1-piperidyl]-[4-(trifluoromethoxy)phenyl]methanone;
[4-[2-(2-fluoro-5-hydroxy-phenyl)-3H-imidazo[4,5-b]pyridin-7-yl]-1-piperidyl]-[4-(trifluoromethoxy)phenyl]methanone;
N-[3-[7-[1-[4-(trifluoromethoxy)benzoyl]-4-piperidyl]-3H-imidazo[4,5-b]pyridin-2-yl]phenyl]tetrahydrofuran-2-carboxamide;
[4-[2-[5-(hydroxymethyl)isoxazol-3-yl]-3H-imidazo[4,5-b]pyridin-7-yl]-1-piperidyl]-[4-(trifluoromethoxy)phenyl]methanone;
1-ethyl-3-hydroxy-6-[7-[1-[4-(trifluoromethoxy)benzoyl]-4-piperidyl]-3H-imidazo[4,5-b]pyridin-2-yl]quinoxalin-2-one;
[3-(2-tetrahydropyran-4-yl-3H-imidazo[4,5-b]pyridin-7-yl)pyrrolidin-1-yl]-[4-(trifluoromethoxy)phenyl]methanone;
[2-amino-4-(trifluoromethoxy)phenyl]-[3-(2-tetrahydropyran-4-yl-3H-imidazo[4,5-b]pyridin-7-yl)pyrrolidin-1-yl]methanone;
(4-bromophenyl)-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[1,4-dioxan-2-yl]-3H-imidazo[4,5-b]pyridin-7-yl]-1-piperidyl]methanone;
4-[7-[1-[2-amino-4-(trifluoromethoxy)benzoyl]-4-piperidyl]-3H-imidazo[4,5-b]pyridin-2-yl]cyclohexanone;
[4-(2-tetrahydropyran-4-yl-3H-imidazo[4,5-b]pyridin-7-yl)-1-piperidyl]-(4-trimethylsilylphenyl)methanone;
(cis)-[4-[2-(3-hydroxycyclobutyl)-3H-imidazo[4,5-b]pyridin-7-yl]-1-piperidyl]-[4-(trifluoromethoxy)phenyl]methanone;
(trans)-[4-[2-(3-hydroxycyclobutyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-(3-methoxy-1-bicyclo[1.1.1]pentanyl)-3H-imidazo[4,5-b]pyridin-7-yl]-1-piperidyl]methanone;
(trans)-[4-(trifluoromethoxy)phenyl]-[4-[2-(4-methoxycyclohexyl)-3H-imidazo[4,5-b]pyridin-7-yl]-1-piperidyl]methanone;
(cis)-[4-(trifluoromethoxy)phenyl]-[4-[2-(4-methoxycyclohexyl)-3H-imidazo[4,5-b]pyridin-7-yl]-1-piperidyl]methanone;
(trans)-[2-amino-4-(trifluoromethoxy)phenyl]-[4-[2-(4-methoxycyclohexyl)-3H-imidazo[4,5-b]pyridin-7-yl]-1-piperidyl]methanone;
(cis)-[2-amino-4-(trifluoromethoxy)phenyl]-[4-[2-(4-methoxycyclohexyl)-3H-imidazo[4,5-b]pyridin-7-yl]-1-piperidyl]methanone;
(2,2-difluoro-1,3-benzodioxol-5-yl)-[4-(2-tetrahydropyran-4-yl-3H-imidazo[4,5-b]pyridin-7-yl)-1-piperidyl]methanone;
(4-bromo-1H-indol-7-yl)-[4-(2-tetrahydropyran-4-yl-3H-imidazo[4,5-b]pyridin-7-yl)-1-piperidyl]methanone;
[4-(2-tetrahydropyran-4-yl-3H-imidazo[4,5-b]pyridin-7-yl)-1-piperidyl]-[2-(trifluoromethyl)-1H-indol-6-yl]methanone;
[4-(cyclopropoxy)phenyl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[5,5-difluoro-3-piperidyl]-3H-imidazo[4,5-b]pyridin-7-yl]-1-piperidyl]methanone;
[2-amino-4-(trifluoromethoxy)phenyl]-[3-(2-tetrahydropyran-4-yl-3H-imidazo[4,5-b]pyridin-7-yl)pyrrolidin-1-yl]methanone;
2-tetrahydropyran-4-yl-7-[1-[4-(trifluoromethoxy)phenyl]sulfonyl-4-piperidyl]-3H-imidazo[4,5-b]pyridine;
[2-amino-4-(trifluoromethoxy)phenyl]-[4-[2-[oxepan-4-yl]-3H-imidazo[4,5-b]pyridin-7-yl]-1-piperidyl]methanone;
[4-[2-[rac-(2R,3S)-3-aminotetrahydrofuran-2-yl]-3H-imidazo[4,5-b]pyridin-7-yl]-1-piperidyl]-[4-(trifluoromethoxy)phenyl]methanone;
(cis)-[2-amino-4-(trifluoromethoxy)phenyl]-[4-[2-(3-hydroxycyclobutyl)-3H-imidazo[4,5-b]pyridin-7-yl]-1-piperidyl]methanone;
(trans)-[2-amino-4-(trifluoromethoxy)phenyl]-[4-[2-(3-hydroxycyclobutyl)-3H-imidazo[4,5-b]pyridin-7-yl]-1-piperidyl]methanone;
[3-(2-tetrahydropyran-4-yl-3H-imidazo[4,5-b]pyridin-7-yl)pyrrolidin-1-yl]-[4-(trifluoromethoxy)phenyl]methanone;
[2-amino-4-(trifluoromethoxy)phenyl]-[4-(2-morpholino-3H-imidazo[4,5-b]pyridin-7-yl)-1-piperidyl]methanone;
[2-amino-4-(trifluoromethoxy)phenyl]-[4-[2-(2-methylmorpholin-2-yl)-3H-imidazo[4,5-b]pyridin-7-yl]-1-piperidyl]methanone;
[4-[2-[6,6-dimethylmorpholin-2-yl]-3H-imidazo[4,5-b]pyridin-7-yl]-1-piperidyl]-[4-(trifluoromethoxy)phenyl]methanone;
[4-[2-[6,6-dimethylmorpholin-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[tetrahydrofuran-2-yl]-3H-imidazo[4,5-b]pyridin-7-yl]-1-piperidyl]methanone;
[4-[2-(7-oxa-4-azaspiro[2.5]octan-6-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[6,6-dimethylmorpholin-2-yl]-3H-imidazo[4,5-b]pyridin-7-yl]-1-piperidyl]methanone;
[2-amino-4-(trifluoromethoxy)phenyl]-[4-[2-[6,6-dimethylmorpholin-2-yl]-3H-imidazo[4,5-b]pyridin-7-yl]-1-piperidyl]methanone;
[4-[2-[1-(oxetan-3-yl)-4-piperidyl]-3H-imidazo[4,5-b]pyridin-7-yl]-1-piperidyl]-[4-(trifluoromethoxy)phenyl]methanone;
[4-[2-(2-oxabicyclo[2.2.2]octan-4-yl)-3H-imidazo[4,5-b]pyridin-7-yl]-1-piperidyl]-[4-(trifluoromethoxy)phenyl]methanone;
[4-(2-morpholino-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-[tetrahydrofuran-3-yl]-3H-imidazo[4,5-b]pyridin-7-yl]-1-piperidyl]methanone;
[2-amino-4-(trifluoromethoxy)phenyl]-[4-[2-[1-(oxetan-3-yl)-4-piperidyl]-3H-imidazo[4,5-b]pyridin-7-yl]-1-piperidyl]methanone;
[2-amino-3-fluoro-4-(trifluoromethoxy)phenyl]-[4-(2-tetrahydropyran-4-yl-3H-imidazo[4,5-b]pyridin-7-yl)-1-piperidyl]methanone;
[4-[2-[rac-(2R,3S)-3-aminotetrahydrofuran-2-yl]-3H-imidazo[4,5-b]pyridin-7-yl]-1-piperidyl]-[2-amino-4-(trifluoromethoxy)phenyl]methanone;
(cis)-[4-(2-tetrahydropyran-4-yl-3H-imidazo[4,5-b]pyridin-7-yl)cyclohexyl]-[4-(trifluoromethoxy)-1-piperidyl]methanone;
(trans)-[4-(2-tetrahydropyran-4-yl-3H-imidazo[4,5-b]pyridin-7-yl)cyclohexyl]-[4-(trifluoromethoxy)-1-piperidyl]methanone;
[4-[2-(4-amino-1-methyl-2-oxabicyclo[2.1.1]hexan-3-yl)-3H-imidazo[4,5-b]pyridin-7-yl]-1-piperidyl]-[2-amino-4-(trifluoromethoxy)phenyl]methanone;
(trans)-[4-[2-(3-methoxycyclobutyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-(7-oxa-4-azaspiro[2.5]octan-6-yl)-3H-imidazo[4,5-b]pyridin-7-yl]-1-piperidyl]methanone;
[2-amino-4-(trifluoromethoxy)phenyl]-[4-[2-(2-oxabicyclo[2.2.2]octan-4-yl)-3H-imidazo[4,5-b]pyridin-7-yl]-1-piperidyl]methanone;
[4-(2-cyclopent-3-en-1-yl-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone;
(2-amino-4-bromo-phenyl)-[4-(2-tetrahydropyran-4-yl-3H-imidazo[4,5-b]pyridin-7-yl)-1-piperidyl]methanone;
[4-[2-(4-amino-1-methyl-2-oxabicyclo[2.1.1]hexan-3-yl)-3H-imidazo[4,5-b]pyridin-7-yl]-1-piperidyl]-[4-(trifluoromethoxy)phenyl]methanone;
N-[2-[4-(2-tetrahydropyran-4-yl-3H-imidazo[4,5-b]pyridin-7-yl)piperidine-1-carbonyl]-5-(trifluoromethoxy)phenyl]acetamide;
(trans)-[4-[2-(3-methoxycyclobutyl)-3H-imidazo[4,5-b]pyridin-7-yl]-1-piperidyl]-[4-(trifluoromethoxy)phenyl]methanone;
[4-[2-(1,2,3,6-tetrahydropyridin-5-yl)-3H-imidazo[4,5-b]pyridin-7-yl]-1-piperidyl]-[4-(trifluoromethoxy)phenyl]methanone;
[4-[2-[4-phenylpyrrolidin-3-yl]-3H-imidazo[4,5-b]pyridin-7-yl]-1-piperidyl]-[4-(trifluoromethoxy)phenyl]methanone;
[4-[2-[4-fluoropyrrolidin-2-yl]-3H-imidazo[4,5-b]pyridin-7-yl]-1-piperidyl]-[4-(trifluoromethoxy)phenyl]methanone;
[4-[2-(2-amino-3,3,3-trifluoro-propyl)-3H-imidazo[4,5-b]pyridin-7-yl]-1-piperidyl]-[4-(trifluoromethoxy)phenyl]methanone;
[4-[2-[6-(aminomethyl)-3-pyridyl]-3H-imidazo[4,5-b]pyridin-7-yl]-1-piperidyl]-[4-(trifluoromethoxy)phenyl]methanone;
[4-[2-[rac-(3R,4S)-4-(4-pyridyl)pyrrolidin-3-yl]-3H-imidazo[4,5-b]pyridin-7-yl]-1-piperidyl]-[4-(trifluoromethoxy)phenyl]methanone;
[4-[2-(3-amino-2-bicyclo[2.2.1]hept-5-enyl)-3H-imidazo[4,5-b]pyridin-7-yl]-1-piperidyl]-[4-(trifluoromethoxy)phenyl]methanone;
[4-[2-[pyrrolidin-2-yl]-3H-imidazo[4,5-b]pyridin-7-yl]-1-piperidyl]-[4-(trifluoromethoxy)phenyl]methanone;
[4-[2-(3-amino-2-thienyl)-3H-imidazo[4,5-b]pyridin-7-yl]-1-piperidyl]-[4-(trifluoromethoxy)phenyl]methanone;
4-[7-[1-[4-(trifluoromethoxy)benzoyl]-4-piperidyl]-3H-imidazo[4,5-b]pyridin-2-yl]piperidine-4-carbonitrile;
[4-[2-[2-piperidyl]-3H-imidazo[4,5-b]pyridin-7-yl]-1-piperidyl]-[4-(trifluoromethoxy)phenyl]methanone;
[4-[2-(4-aminotetrahydropyran-4-yl)-3H-imidazo[4,5-b]pyridin-7-yl]-1-piperidyl]-[4-(trifluoromethoxy)phenyl]methanone;
[4-[2-(3-amino-4-methoxy-phenyl)-3H-imidazo[4,5-b]pyridin-7-yl]-1-piperidyl]-[4-(trifluoromethoxy)phenyl]methanone;
[4-[2-(2-piperidyl)-3H-imidazo[4,5-b]pyridin-7-yl]-1-piperidyl]-[4-(trifluoromethoxy)phenyl]methanone;
[4-[2-[pyrrolidin-2-yl]-3H-imidazo[4,5-b]pyridin-7-yl]-1-piperidyl]-[4-(trifluoromethoxy)phenyl]methanone;
[4-[2-(1,2,3,4-tetrahydroisoquinolin-6-yl)-3H-imidazo[4,5-b]pyridin-7-yl]-1-piperidyl]-[4-(trifluoromethoxy)phenyl]methanone;
[4-[2-(3-amino-5-hydroxy-phenyl)-3H-imidazo[4,5-b]pyridin-7-yl]-1-piperidyl]-[4-(trifluoromethoxy)phenyl]methanone;
[4-[2-(1H-indol-3-yl)-3H-imidazo[4,5-b]pyridin-7-yl]-1-piperidyl]-[4-(trifluoromethoxy)phenyl]methanone;
[4-[2-(4,5,6,7-tetrahydrothieno[3,2-c]pyridin-2-yl)-3H-imidazo[4,5-b]pyridin-7-yl]-1-piperidyl]-[4-(trifluoromethoxy)phenyl]methanone;
[4-[2-(azetidin-2-yl)-3H-imidazo[4,5-b]pyridin-7-yl]-1-piperidyl]-[4-(trifluoromethoxy)phenyl]methanone;2,2,2-trifluoroacetic acid
[4-[2-(3-oxa-9-azabicyclo[3.3.1]nonan-7-yl)-3H-imidazo[4,5-b]pyridin-7-yl]-1-piperidyl]-[4-(trifluoromethoxy)phenyl]methanone;
[4-[2-(4-amino-3-pyridyl)-3H-imidazo[4,5-b]pyridin-7-yl]-1-piperidyl]-[4-(trifluoromethoxy)phenyl]methanone;
[4-[2-[piperazin-2-yl]-3H-imidazo[4,5-b]pyridin-7-yl]-1-piperidyl]-[4-(trifluoromethoxy)phenyl]methanone;
[4-[2-(3-aminocyclobutyl)-3H-imidazo[4,5-b]pyridin-7-yl]-1-piperidyl]-[4-(trifluoromethoxy)phenyl]methanone;
[4-[2-[3-(methylamino)phenyl]-3H-imidazo[4,5-b]pyridin-7-yl]-1-piperidyl]-[4-(trifluoromethoxy)phenyl]methanone;
[4-[2-(2-azabicyclo[4.1.0]heptan-7-yl)-3H-imidazo[4,5-b]pyridin-7-yl]-1-piperidyl]-[4-(trifluoromethoxy)phenyl]methanone;
[4-[2-[4-hydroxy-6-(trifluoromethyl)-3-quinolyl]-3H-imidazo[4,5-b]pyridin-7-yl]-1-piperidyl]-[4-(trifluoromethoxy)phenyl]methanone;
[4-[2-(3-oxabicyclo[3.1.0]hexan-6-yl)-3H-imidazo[4,5-b]pyridin-7-yl]-1-piperidyl]-[4-(trifluoromethoxy)phenyl]methanone;
N-[4-hydroxy-2-[7-[1-[4-(trifluoromethoxy)benzoyl]-4-piperidyl]-3H-imidazo[4,5-b]pyridin-2-yl]phenyl]acetamide;
5,6-dimethyl-3-[7-[1-[4-(trifluoromethoxy)benzoyl]-4-piperidyl]-3H-imidazo[4,5-b]pyridin-2-yl]-1H-pyridin-2-one;
1-[3,5-dimethyl-4-[7-[1-[4-(trifluoromethoxy)benzoyl]-4-piperidyl]-3H-imidazo[4,5-b]pyridin-2-yl]-1H-pyrrol-2-yl]ethanone;
[4-[2-[1-(1,1-dioxothiolan-3-yl)-4-piperidyl]-3H-imidazo[4,5-b]pyridin-7-yl]-1-piperidyl]-[4-(trifluoromethoxy)phenyl]methanone;
[4-[2-(1-methylsulfonylazetidin-3-yl)-3H-imidazo[4,5-b]pyridin-7-yl]-1-piperidyl]-[4-(trifluoromethoxy)phenyl]methanone;
[2-hydroxy-4-(trifluoromethoxy)phenyl]-[4-(2-tetrahydropyran-4-yl-3H-imidazo[4,5-b]pyridin-7-yl)-1-piperidyl]methanone;
[4-[2-(1,4-oxazepan-2-yl)-3H-imidazo[4,5-b]pyridin-7-yl]-1-piperidyl]-[4-(trifluoromethoxy)phenyl]methanone;
[4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone;
[4-(1,1,2,2-tetrafluoroethoxy)phenyl]-[4-(2-tetrahydropyran-4-yl-3H-imidazo[4,5-b]pyridin-7-yl)-1-piperidyl]methanone;
[4-(1,1,2,2,2-pentafluoroethoxy)phenyl]-[4-(2-tetrahydropyran-4-yl-3H-imidazo[4,5-b]pyridin-7-yl)-1-piperidyl]methanone;
[4-(2-phenyl-3H-imidazo[4,5-b]pyridin-7-yl)-1-piperidyl]-[4-(trifluoromethoxy)phenyl]methanone;
(2,2,3,3-tetrafluoro-1,4-benzodioxin-6-yl)-[4-(2-tetrahydropyran-4-yl-3H-imidazo[4,5-b]pyridin-7-yl)-1-piperidyl]methanone;
[4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[7-oxa-4-azaspiro[2.5]octan-6-yl]-3H-imidazo[4,5-b]pyridin-7-yl]-1-piperidyl]methanone;
[4-[2-[rac-(1S,3S)-3-aminocyclohexyl]-3H-imidazo[4,5-b]pyridin-7-yl]-1-piperidyl]-[4-(trifluoromethoxy)phenyl]methanone;
[4-(2-cyclopentyl-3H-imidazo[4,5-b]pyridin-7-yl)-1-piperidyl]-[4-(trifluoromethoxy)phenyl]methanone;
[4-[2-(4-methylpiperazin-1-yl)-3H-imidazo[4,5-b]pyridin-7-yl]-1-piperidyl]-[4-(trifluoromethoxy)phenyl]methanone;
[4-[2-[7-oxa-4-azaspiro[2.5]octan-6-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-(2-cyclopent-3-en-1-yl-3H-imidazo[4,5-b]pyridin-7-yl)-1-piperidyl]methanone;
[4-[2-(azepan-3-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-(2-cyclopentyl-3H-imidazo[4,5-b]pyridin-7-yl)-1-piperidyl]methanone;
[4-[2-[4-amino-1-methyl-2-oxabicyclo[2.1.1]hexan-3-yl]-3H-imidazo[4,5-b]pyridin-7-yl]-1-piperidyl]-[2-amino-4-(trifluoromethoxy)phenyl]methanone;
[4-[2-(3-hydroxyphenyl)-3H-imidazo[4,5-b]pyridin-7-yl]-1-piperidyl]-[4-(trifluoromethoxy)phenyl]methanone;
[4-[2-[rac-(1R,3S)-3-aminocyclohexyl]-3H-imidazo[4,5-b]pyridin-7-yl]-1-piperidyl]-[4-(trifluoromethoxy)phenyl]methanone;
[4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[4-(2-tetrahydropyran-4-yl-3H-imidazo[4,5-b]pyridin-7-yl)azepan-1-yl]methanone;
[4-[2-(3-fluoro-4-piperidyl)-3H-imidazo[4,5-b]pyridin-7-yl]-1-piperidyl]-[4-(trifluoromethoxy)phenyl]methanone;
[4-[2-(1,2,3,6-tetrahydropyridin-4-yl)-3H-imidazo[4,5-b]pyridin-7-yl]-1-piperidyl]-[4-(trifluoromethoxy)phenyl]methanone;
3-[7-[1-[4-(trifluoromethoxy)benzoyl]-4-piperidyl]-3H-imidazo[4,5-b]pyridin-2-yl]bicyclo[1.1.1]pentane-1-carboxylic acid;
3-[4-(2-tetrahydropyran-4-yl-3H-imidazo[4,5-b]pyridin-7-yl)piperidine-1-carbonyl]-6-(trifluoromethyl)-1H-pyridin-2-one;
[4-(difluoromethoxy)phenyl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[1,1-dioxothiolan-3-yl]-3H-imidazo[4,5-b]pyridin-7-yl]-1-piperidyl]methanone;
[4-[2-[1,4-oxazepan-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[1,4-oxazepan-2-yl]-3H-imidazo[4,5-b]pyridin-7-yl]-1-piperidyl]methanone;
[4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[4-(2-tetrahydropyran-4-yl-3H-imidazo[4,5-b]pyridin-7-yl)azepan-1-yl]methanone;
(trans)-4-[7-[1-[4-(trifluoromethoxy)benzoyl]-4-piperidyl]-3H-imidazo[4,5-b]pyridin-2-yl]cyclohexanecarboxylic acid;
(cis)-[4-[2-[3-aminotetrahydrofuran-2-yl]-3H-imidazo[4,5-b]pyridin-7-yl]-1-piperidyl]-[2-amino-4-(trifluoromethoxy)phenyl]methanone;
[4-[2-(1,1-dioxothiolan-3-yl)-3H-imidazo[4,5-b]pyridin-7-yl]-1-piperidyl]-[4-(trifluoromethoxy)phenyl]methanone;
[4-[2-(4-hydroxy-4-methyl-cyclohexyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(trifluoromethoxy)phenyl]-[4-[2-(4-hydroxy-4-methyl-cyclohexyl)-3H-imidazo[4,5-b]pyridin-7-yl]-1-piperidyl]methanone;
[2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(trifluoromethoxy)phenyl]-[4-[2-(1,2,3,6-tetrahydropyridin-4-yl)-3H-imidazo[4,5-b]pyridin-7-yl]-1-piperidyl]methanone;
[4-[2-[azepan-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[azepan-3-yl]-3H-imidazo[4,5-b]pyridin-7-yl]-1-piperidyl]methanone;
[2-amino-4-(pentafluoro-λ⁶-sulfanyl)phenyl]-[4-(2-cyclopentyl-3H-imidazo[4,5-b]pyridin-7-yl)-1-piperidyl]methanone;
[4-(2-cyclopentyl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydrofuran-2-yl-3H-imidazo[4,5-b]pyridin-7-yl)-1-piperidyl]methanone;
(cis)-[4-[2-(4-methoxycyclohexyl)-3H-imidazo[4,5-b]pyridin-7-yl]-1-piperidyl]-[4-(pentafluoro-λ⁶-sulfanyl)phenyl]methanone;
(trans)-[4-[2-(4-methoxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
(cis)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone;
(trans)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone;
[4-(pentafluoro-λ⁶-sulfanyl)phenyl]-[4-[2-[tetrahydrofuran-2-yl]-3H-imidazo[4,5-b]pyridin-7-yl]-1-piperidyl]methanone;
[4-(pentafluoro-λ⁶-sulfanyl)phenyl]-[4-[2-[tetrahydrofuran-3-yl]-3H-imidazo[4,5-b]pyridin-7-yl]-1-piperidyl]methanone;
[4-[2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[4-(pentafluoro-λ⁶-sulfanyl)phenyl]-[4-[2-(4-piperidyl)-3H-imidazo[4,5-b]pyridin-7-yl]-1-piperidyl]methanone;hydrochloride;
[4-[2-[morpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(3-methoxy-1-bicyclo[1.1.1]pentanyl)-3H-imidazo[4,5-b]pyridin-7-yl]-1-piperidyl]methanone;
[4-(2-N-morpholino-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-(4-piperidyl)-3H-imidazo[4,5-b]pyridin-7-yl]-1-piperidyl]methanone;
[2-amino-4-(pentafluoro-λ⁶-sulfanyl)phenyl]-[4-[2-[tetrahydrofuran-3-yl]-3H-imidazo[4,5-b]pyridin-7-yl]-1-piperidyl]methanone;
(cis)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone;
(trans)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone;
[2-amino-4-(pentafluoro-λ⁶-sulfanyl)phenyl]-[4-(2-N-morpholino-3H-imidazo[4,5-b]pyridin-7-yl)-1-piperidyl]methanone;
(trans)-[4-[2-(3-methoxycyclobutyl)-3H-imidazo[4,5-b]pyridin-7-yl]-1-piperidyl]-[4-(pentafluoro-λ⁶-sulfanyl)phenyl]methanone;
[4-[2-(1-methyl-4-piperidyl)-3H-imidazo[4,5-b]pyridin-7-yl]-1-piperidyl]-[4-(pentafluoro-λ⁶-sulfanyl)phenyl]methanone;
[4-[2-(1-cyclopropyl-4-piperidyl)-3H-imidazo[4,5-b]pyridin-7-yl]-1-piperidyl]-[4-(pentafluoro-λ⁶-sulfanyl)phenyl]methanone;
[4-[2-[morpholin-2-yl]-3H-imidazo[4,5-b]pyridin-7-yl]-1-piperidyl]-[4-(pentafluoro-λ⁶-sulfanyl)phenyl]methanone;
(cis)-[4-[2-(3-methoxycyclobutyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(1-methyl-4-piperidyl)-3H-imidazo[4,5-b]pyridin-7-yl]-1-piperidyl]methanone;
[2-amino-4-(pentafluoro-λ⁶-sulfanyl)phenyl]-[4-[2-[morpholin-2-yl]-3H-imidazo[4,5-b]pyridin-7-yl]-1-piperidyl]methanone;
[2-amino-4-(pentafluoro-λ⁶-sulfanyl)phenyl]-[4-[2-(1-cyclopropyl-4-piperidyl)-3H-imidazo[4,5-b]pyridin-7-yl]-1-piperidyl]methanone;
[4-[2-(1-methoxy-1-methyl-ethyl)-3H-imidazo[4,5-b]pyridin-7-yl]-1-piperidyl]-[4-(pentafluoro-λ⁶-sulfanyl)phenyl]methanone;
[2-(methylamino)-4-(pentafluoro-λ⁶-sulfanyl)phenyl]-[4-[2-(1-methyl-4-piperidyl)-3H-imidazo[4,5-b]pyridin-7-yl]-1-piperidyl]methanone;
[2-amino-4-(pentafluoro-λ⁶-sulfanyl)phenyl]-[4-[2-(1-methoxy-1-methyl-ethyl)-3H-imidazo[4,5-b]pyridin-7-yl]-1-piperidyl]methanone;
(cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
[4-[2-[4-methylmorpholin-2-yl]-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
(cis)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[morpholin-2-yl]-3H-imidazo[4,5-b]pyridin-7-yl]-1-piperidyl]methanone;
(trans)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone;
[4-[2-[4-methylmorpholin-2-yl]-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone;
[4-(pentafluoro-λ⁶-sulfanyl)phenyl]-[4-(2-tetrahydropyran-4-yl-3H-imidazo[4,5-b]pyridin-7-yl)azepan-1-yl]methanone;
[4-[2-[2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone;
3-methyl-3-[7-[1-[4-(pentafluoro-λ⁶-sulfanyl)benzoyl]-4-piperidyl]-3H-imidazo[4,5-b]pyridin-2-yl]cyclobutanone;
[4-[2-(1-hydroxy-1-methyl-ethyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(1-hydroxy-1-methyl-ethyl)-3H-imidazo[4,5-b]pyridin-7-yl]-1-piperidyl]methanone;
[(cis)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[(trans)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-[2-oxa-5-azabicyclo[2.2.1]heptan-1-yl]-3H-imidazo[4,5-b]pyridin-7-yl]-1-piperidyl]methanone;
[2-amino-4-(trifluoromethoxy)phenyl]-[4-fluoro-4-(6-fluoro-2-tetrahydropyran-4-yl-3H-imidazo[4,5-b]pyridin-7-yl)-1-piperidyl]methanone;
[4-[2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(3-hydroxy-1-bicyclo[1.1.1]pentanyl)-3H-imidazo[4,5-b]pyridin-7-yl]-1-piperidyl]methanone;
[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[2-hydroxy-4-(pentafluoro-λ⁶-sulfanyl)phenyl]-[4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[4-(6-fluoro-2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(cis)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(trans)-2-methyl-4-(2-tetrahydropyran-4-yl-3H-imidazo[4,5-b]pyridin-7-yl)-1-piperidyl]methanone;
(cis)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone;
(trans)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone;
[4-(6-fluoro-2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(6-fluoro-2-morpholin-2-yl-3H-imidazo[4,5-b]pyridin-7-yl)-1-piperidyl]-[4-(pentafluoro-λ⁶-sulfanyl)phenyl]methanone;
[4-[6-fluoro-2-(2-oxabicyclo[2.2.2]octan-4-yl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(2-oxabicyclo[2.2.2]octan-4-yl)-3H-imidazo[4,5-b]pyridin-7-yl]-1-piperidyl]methanone;
[4-(6-fluoro-2-tetrahydropyran-3-yl-3H-imidazo[4,5-b]pyridin-7-yl)-1-piperidyl]-[4-(pentafluoro-λ⁶-sulfanyl)phenyl]methanone;
(cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
[2-fluoro-4-(pentafluoro-λ⁶-sulfanyl)phenyl]-[4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[4-(6-fluoro-2-tetrahydropyran-3-yl-3H-imidazo[4,5-b]pyridin-7-yl)-1-piperidyl]methanone;
(trans)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone;
(cis)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone;
[4-(6-fluoro-2-tetrahydrofuran-3-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(oxepan-4-yl)-3H-imidazo[4,5-b]pyridin-7-yl]-1-piperidyl]methanone;
[4-[6-fluoro-2-(oxepan-4-yl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(6-fluoro-2-tetrahydrofuran-3-yl-3H-imidazo[4,5-b]pyridin-7-yl)-1-piperidyl]methanone;
[4-[2-[6-methylmorpholin-2-yl]-3H-imidazo[4,5-b]pyridin-7-yl]-1-piperidyl]-[4-(pentafluoro-λ⁶-sulfanyl)phenyl]methanone;
[4-[6-fluoro-2-(1-methoxy-1-methyl-ethyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(pentafluoro-λ⁶-sulfanyl)phenyl]-[4-[6-fluoro-2-(3-methoxy-1-bicyclo[1.1.1]pentanyl)-3H-imidazo[4,5-b]pyridin-7-yl]-1-piperidyl]methanone;
[4-[6-fluoro-2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(6-fluoro-2-tetrahydrofuran-2-yl-3H-imidazo[4,5-b]pyridin-7-yl)-1-piperidyl]methanone;
[4-(6-fluoro-2-tetrahydrofuran-2-yl-3H-imidazo[4,5-b]pyridin-7-yl)-1-piperidyl]-[4-(pentafluoro-λ⁶-sulfanyl)phenyl]methanone;
[4-(6-fluoro-2-tetrahydropyran-4-yl-3H-imidazo[4,5-b]pyridin-7-yl)-1-piperidyl]-[2-hydroxy-4-(pentafluoro-λ⁶-sulfanyl)phenyl]methanone;
[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone;
[2-amino-4-(pentafluoro-λ⁶-sulfanyl)phenyl]-[4-[2-[4-(hydroxymethyl)-1-piperidyl]-3H-imidazo[4,5-b]pyridin-7-yl]-1-piperidyl]methanone;
[4-[6-fluoro-2-(3-methoxycyclobutyl)-3H-imidazo[4,5-b]pyridin-7-yl]-1-piperidyl]-[4-(pentafluoro-λ⁶-sulfanyl)phenyl]methanone;
[4-(6-fluoro-2-tetrahydropyran-2-yl-3H-imidazo[4,5-b]pyridin-7-yl)-1-piperidyl]-[4-(pentafluoro-λ⁶-sulfanyl)phenyl]methanone;
(trans)-[4-[6-fluoro-2-(4-hydroxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
[4-[6-fluoro-2-[2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
[4-[2-[3-methoxycyclohexyl]-3H-imidazo[4,5-b]pyridin-7-yl]-1-piperidyl]-[4-(pentafluoro-λ⁶-sulfanyl)phenyl]methanone;
[4-(6-fluoro-2-thiomorpholin-2-yl-3H-imidazo[4,5-b]pyridin-7-yl)-1-piperidyl]-[4-(pentafluoro-λ⁶-sulfanyl)phenyl]methanone;
(trans)-[4-[6-fluoro-2-(4-methoxycyclohexyl)-3H-imidazo[4,5-b]pyridin-7-yl]-1-piperidyl]-[2-hydroxy-4-(pentafluoro-λ⁶-sulfanyl)phenyl]methanone;
[2-amino-4-(pentafluoro-λ⁶-sulfanyl)phenyl]-[4-[6-fluoro-2-(1-methoxy-1-methyl-ethyl)-3H-imidazo[4,5-b]pyridin-7-yl]-1-piperidyl]methanone;
[4-[6-fluoro-2-[tetrahydrofuran-3-yl]-3H-imidazo[4,5-b]pyridin-7-yl]-1-piperidyl]-[4-(pentafluoro-λ⁶-sulfanyl)phenyl]methanone;
[4-[6-fluoro-2-[6-methylmorpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[tetrahydrofuran-3-yl]-3H-imidazo[4,5-b]pyridin-7-yl]-1-piperidyl]methanone;
[4-[6-fluoro-2-[morpholin-2-yl]-3H-imidazo[4,5-b]pyridin-7-yl]-1-piperidyl]-[4-(pentafluoro-λ⁶-sulfanyl)phenyl]methanone;
5-[6-fluoro-7-[1-[4-(pentafluoro-λ⁶-sulfanyl)benzoyl]-4-piperidyl]-3H-imidazo[4,5-b]pyridin-2-yl]piperidin-2-one;
(trans)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(2-cyclopentyl-6-fluoro-3H-imidazo[4,5-b]pyridin-7-yl)-1-piperidyl]methanone;
[2-amino-4-(pentafluoro-λ⁶-sulfanyl)phenyl]-[4-[2-[3-methoxycyclohexyl]-3H-imidazo[4,5-b]pyridin-7-yl]-1-piperidyl]methanone;
[4-[6-fluoro-2-[oxepan-4-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[oxepan-4-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[tetrahydrofuran-2-yl]-3H-imidazo[4,5-b]pyridin-7-yl]-1-piperidyl]methanone;
[4-[6-fluoro-2-[tetrahydrofuran-2-yl]-3H-imidazo[4,5-b]pyridin-7-yl]-1-piperidyl]-[4-(pentafluoro-λ⁶-sulfanyl)phenyl]methanone;
[4-[6-fluoro-2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(3-hydroxy-1-bicyclo[1.1.1]pentanyl)-3H-imidazo[4,5-b]pyridin-7-yl]-1-piperidyl]methanone;
(trans)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
[4-(2-cyclopentyl-6-fluoro-3H-imidazo[4,5-b]pyridin-7-yl)-1-piperidyl]-[4-(pentafluoro-λ⁶-sulfanyl)phenyl]methanone;
[4-[6-fluoro-2-(3-methoxy-1-bicyclo[1.1.1]pentanyl)-3H-imidazo[4,5-b]pyridin-7-yl]-1-piperidyl]-[4-(trifluoromethoxy)phenyl]methanone;
[4-[6-fluoro-2-(3-methoxy-1-bicyclo[1.1.1]pentanyl)-3H-imidazo[4,5-b]pyridin-7-yl]-1-piperidyl]-[2-hydroxy-4-(pentafluoro-λ⁶-sulfanyl)phenyl]methanone;
[2-amino-4-(trifluoromethoxy)phenyl]-[4-[6-fluoro-2-(3-methoxy-1-bicyclo[1.1.1]pentanyl)-3H-imidazo[4,5-b]pyridin-7-yl]-1-piperidyl]methanone;
[4-[6-fluoro-2-(2-oxabicyclo[2.2.2]octan-4-yl)-3H-imidazo[4,5-b]pyridin-7-yl]-1-piperidyl]-[4-(trifluoromethoxy)phenyl]methanone;
[2-amino-5-(pentafluoro-λ⁶-sulfanyl)phenyl]-[4-(2-tetrahydropyran-4-yl-3H-imidazo[4,5-b]pyridin-7-yl)-1-piperidyl]methanone;
[4-[2-(1-cyclopropyl-4-piperidyl)-6-fluoro-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-[3-(trifluoromethyl)-1-bicyclo[1.1.1]pentanyl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[tetrahydropyran-2-yl]-3H-imidazo[4,5-b]pyridin-7-yl]-1-piperidyl]methanone;
[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;
[4-[6-fluoro-2-[3-(trifluoromethyl)-1-bicyclo[1.1.1]pentanyl]-3H-imidazo[4,5-b]pyridin-7-yl]-1-piperidyl]-[4-(trifluoromethoxy)phenyl]methanone;
[4-[2-(1-methoxy-1-methyl-ethyl)-3H-imidazo[4,5-b]pyridin-7-yl]-1-piperidyl]-[4-(trifluoromethoxy)phenyl]methanone;
[4-[6-fluoro-2-[tetrahydropyran-2-yl]-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone;
(cis)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone;
(cis)-[4-[6-fluoro-2-(4-methoxycyclohexyl)-3H-imidazo[4,5-b]pyridin-7-yl]-1-piperidyl]-[4-(trifluoromethoxy)phenyl]methanone;
(trans)-[4-[6-fluoro-2-(4-methoxycyclohexyl)-3H-imidazo[4,5-b]pyridin-7-yl]-1-piperidyl]-[4-(trifluoromethoxy)phenyl]methanone;
[4-[6-fluoro-2-(1-methoxy-1-methyl-ethyl)-3H-imidazo[4,5-b]pyridin-7-yl]-1-piperidyl]-[4-(trifluoromethoxy)phenyl]methanone;
(trans)-(4-(6-fluoro-2-(4-hydroxy-4-methylcyclohexyl)-3H-imidazo[4,5-b]pyridin-7-yl)piperidin-1-yl)(4-(pentafluoro-λ⁶-sulfanyl)phenyl)methanone;
(cis)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-(1-methoxy-1-methyl-ethyl)-3H-imidazo[4,5-b]pyridin-7-yl]-1-piperidyl]methanone;
(trans)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone;
4-(6-fluoro-2-N-morpholino-3H-imidazo[4,5-b]pyridin-7-yl)-1-piperidyl]-[4-(pentafluoro-λ⁶-sulfanyl)phenyl]methanone;
(trans)-[4-[6-fluoro-2-[4-hydroxy-4-(trifluoromethyl)cyclohexyl]-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-(2-oxabicyclo[2.2.2]octan-4-yl)-3H-imidazo[4,5-b]pyridin-7-yl]-1-piperidyl]methanone;
[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
(cis)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
2-[6-fluoro-7-[1-[4-(pentafluoro-λ6-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylic acid;
[4-[2-(1,1-dioxo-1,4-thiazinan-2-yl)-6-fluoro-3H-imidazo[4,5-b]pyridin-7-yl]-1-piperidyl]-[4-(pentafluoro-λ⁶-sulfanyl)phenyl]methanone;
[4-[6-fluoro-2-(3-methoxycyclobutyl)-3H-imidazo[4,5-b]pyridin-7-yl]-1-piperidyl]-[4-(trifluoromethoxy)phenyl]methanone;
(2-amino-4-(trifluoromethoxy) phenyl]-[4-[6-fluoro-2-(3-methoxycyclobutyl)-3H-imidazo[4,5-b]pyridin-7-yl]-1-piperidyl]methanone;
(cis)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone;
(trans)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone; and
7-[6-fluoro-7-[1-[4-(trifluoromethoxy)benzoyl]-4-piperidyl]-3H-imidazo[4,5-b]pyridin-2-yl]-2-oxa-5-azaspiro[3.5]nonane-5-carboxylic acid, and the pharmaceutically acceptable salts thereof.

A further aspect provides a compound as defined hereinbefore, wherein the compound has an IC₅₀ value of less than about 2 μM, e.g., less than about 1 μM, 0.5 μM, 0.2 μM, 100 nM, or 50 nM, against ERK5.

A further aspect provides a pharmaceutical composition comprising the compound defined hereinbefore and at least one pharmaceutically acceptable excipient or carrier.

A further aspect provides a compound, or a pharmaceutical composition, as defined hereinbefore for use in therapy.

A further aspect provides a compound, or a pharmaceutical composition, as defined hereinbefore for use in the treatment or prevention of cancer.

In embodiments, the cancer is characterized by increased MAPK7 expression and/or increased ERK5 activity.

In embodiments, the cancer is selected from leukaemia, breast cancer, multiple myeloma, colon cancer, renal cell carcinoma, mesothelioma, adenocarcinoma, neuroblastoma, and hepatocellular carcinoma.

### DETAILED DESCRIPTION

Although specific embodiments of the present disclosure will now be described with reference to the description and examples, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety.

The practice of the present disclosure will employ, unless otherwise indicated, conventional techniques of chemical synthesis, tissue culture, immunology, molecular biology, microbiology, cell biology, recombinant DNA, etc., which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press). All numerical designations, e.g., pH, temperature, time, concentration, molecular weight, etc., including ranges, are approximations which are varied (+) or (−) by increments of, e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±1% of the value. It also is to be understood, although not always explicitly stated, that the reagents described herein are merely exemplary and that equivalents of such are known in the art.

As used in the specification and claims, the singular forms “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, without excluding other elements. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass, and to disclose, the corresponding statements in which the term “comprising” is replaced by “consisting essentially of” or “consisting of”.

A “subject,” “individual”, or “patient” is used interchangeably herein, and refers to a vertebrate, such as a mammal. Mammals include, but are not limited to, rodents, farm animals, sport animals, pets, and primates; for example murines, rats, rabbit, simians, bovines, ovines, porcines, canines, felines, equines, and humans. In a particular embodiment, the mammal is a human.

“Administering” is defined herein as a means of providing an agent or a composition containing the agent to a subject in a manner that results in the agent being contacted with (e.g., being inside) the subject's body. Such an administration can be by any route including, without limitation, oral, transdermal (e.g., by the vagina, rectum, or oral mucosa), by injection (e.g., subcutaneous, intravenous, parenteral, intraperitoneal, or into the central nervous system), or by inhalation (e.g., oral or nasal). Administration may also involve providing a substance or composition to a part of the surface of the subject's body, for example by topical administration to the skin. Pharmaceutical preparations are, of course, given by forms suitable for each administration route.

“Treating” or “treatment” of a disease includes: (1) preventing the disease, i.e. causing the clinical symptoms of the disease not to develop in a patient that may be predisposed to the disease but does not yet experience or display symptoms of the disease; (2) inhibiting the disease, i.e. arresting or reducing the development of the disease or its clinical symptoms; and/or (3) relieving the disease, i.e. causing regression of the disease or its clinical symptoms.

The term “suffering” as it relates to the term “treatment” refers to a patient or individual who has been diagnosed with or is predisposed to the disease. A patient may also be referred to being “at risk of suffering” from a disease because of a history of disease in their family lineage or because of the presence of genetic mutations associated with the disease. A patient at risk of a disease has not yet developed all or some of the characteristic pathologies of the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including, for example, the activity of the specific compound employed, the age, body weight, general health, sex, and diet of the subject, the time of administration, the rate of excretion, the drug combination, the severity of the particular disorder being treated and the form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with the condition. The total daily dose may be administered in single or divided doses and may, at the physician's discretion, fall outside of the typical range given herein.

As used herein, the terms “increased” and “elevated” are used interchangeably and encompass any measurable increase in a biological function and/or a biological activity and/or a concentration. For example, an increase can be by at least about 10%, e.g. at least about 15%, 20%, 25%, 30%, 35%, 40%, 45%, 50%, 55%, 60%, 65%, 70%, 75%, 80%, 85%, 90%, or 95%, such as at least about 95%, 96%, 97%, 98%, 99%, or 100%. Thus, an increase can be by at least about 2-fold, 3-fold, 4-fold, 5-fold, 6-fold, 7-fold, 8-fold, 9-fold, or 10-fold, such as at least about 20-fold, 25-fold, 50-fold, 100-fold, or higher, relative to a control or baseline amount or function, or activity, or concentration.

As used herein, the terms “increased expression” and/or “increased activity” of a substance, such as ERK5, in a sample or cancer or patient, typically refers to an increase in the amount of the substance (e.g., of the MAPK7 gene product or ERK5 protein), although it may also denote an increase in the biological activity of the substance (e.g., constitutive activation of phosphorylation and/or reduced discrimination of phosphorylation sites of ERK5). For example, an increase can be by an amount of about 5%, e.g., about 10%, 15%, 20%, 25%, 30%, 35%, 40%, 45%, 50%, 55%, 60%, 65%, 70%, 75%, 80%, 85%, 90%, or 95%, such as about 96%, 97%, 98%, 99%, or 100%. Thus, the increase can be about 2-fold, 3-fold, 4-fold, 5-fold, 6-fold, 7-fold, 8-fold, 9-fold, or 10-fold, such as about 20-fold, 25-fold, 50-fold, 100-fold, or higher, relative to the amount (or activity) of the substance, such as ERK5, in a control sample or control samples, such as an individual or group of individuals who are not suffering from the disease or disorder (e.g. cancer) or an internal control, as determined by techniques known in the art. A subject can also be determined to have an “increased expression” or “increased activity” of ERK5 if the expression and/or activity of ERK5 is increased by one standard deviation, two standard deviations, three standard deviations, four standard deviations, five standard deviations, or more, relative to the mean (average) or median amount of ERK5 in a control group of samples or a baseline group of samples or a retrospective analysis of patient samples. As practiced in the art, such control or baseline expression levels can be previously determined, or measured prior to the measurement in the sample or cancer or subject, or can be obtained from a database of such control samples.

As used herein, the term “pharmaceutically acceptable excipient” encompasses any of the standard pharmaceutical excipients, for example as described in Remington's Pharmaceutical Sciences (20th ed., Mack Publishing Co. 2000). Such excipients include carriers such as a phosphate buffered saline solution, water, and emulsions, such as an oil/water or water/oil emulsion, and various types of wetting agents. Pharmaceutical compositions also can include stabilizers, preservatives, adjuvants, fillers, binders, lubricants, and the like.

As used herein, the term “alkyl” means a saturated linear or branched free radical consisting essentially of carbon atoms and a corresponding number of hydrogen atoms. Exemplary alkyl groups include methyl, ethyl, n-propyl, isopropyl, n-butyl, isobutyl, etc. Other alkyl groups will be readily apparent to those of skill in the art given the benefit of the present disclosure. The terms “(C₁-C₃)alkyl”, “(C₁-C₆)alkyl”, etc., have equivalent meanings, i.e., a saturated linear or branched free radical consisting essentially of 1 to 3 (or 1 to 6) carbon atoms and a corresponding number of hydrogen atoms. The definition of “alkyl” also applies in the context of other groups which comprise alkyl groups, such as “O(C₁-C₃)alkyl”. The term “haloalkyl” means an alkyl group which is substituted by one or more halogens. Exemplary haloalkyl groups include trifluoromethyl, trifluoroethyl, difluoroethyl, pentafluoroethyl, chloromethyl, etc. One or more carbon atoms in the backbone of the alkyl group may be substituted by (or bonded to) a heteroatom by a multiple bond (e.g., a double bond); for example, a carbon atom of the alkyl group may be bonded to oxygen via a double bond (i.e., substituted by oxo to provide a carbonyl function). The presence of such a substituent does not prevent the carbon backbone of the free radical being considered as an alkyl group.

As used herein, the term “cyclic group” means a saturated, partially or fully unsaturated, or aromatic group having at least 3 to 10 atoms (i.e., ring atoms) that form a ring. Where a cyclic group is defined as having a certain number of members, the term “members”, “membered” and the like is used to denote the number of ring atoms in said cyclic group. For example, a 5-membered cyclic group (e.g., a 5-membered heterocyclic group) contains 5 ring atoms. It will be appreciated that a cyclic group may be part of a larger cyclic system; for example, bicyclo[4.3.0]nonane comprises two carbocyclic groups, namely a cyclohexane group and a cyclopentane group, which are fused to form the carbocyclic system which makes up the molecule. The term “cyclic group” is intended to encompass both carbocyclic groups as well as heterocyclic groups. The term “carbocyclic” refers to a group having at least 3 to 9 carbon atoms that form a ring. The term “heterocyclic” refers to a group having at least 3 to 10 atoms that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen.

The term “spiro” or “spirocyclic” as used herein in relation to cyclic groups denotes that a first cyclic group within a multicyclic system is attached to a second cyclic group within said multicyclic system, wherein the ring atoms of said first cyclic group and the ring atoms of said second cyclic group have only one atom in common, i.e., said first and second cyclic groups share only one common ring atom. For example, the spiro[5.5]undecanyl group comprises two cyclohexane rings which have a single carbon ring atom in common.

The term “fused” as used herein in relation to cyclic groups denotes that a first cyclic group within a multicyclic system is attached to a second cyclic group within said multicyclic system, wherein the ring atoms of said first cyclic group and the ring atoms of said second cyclic group have two adjacent atoms in common, i.e., said first and second cyclic groups share two common ring atoms. For example, the bicyclo[4.4.0]decanyl group comprises two cyclohexane rings which have two adjacent carbon ring atoms in common.

The term “bridged” as used herein in relation to cyclic groups denotes that a first cyclic group within a multicyclic system is attached to a second cyclic group within said multicyclic system, wherein the ring atoms of said first cyclic group and the ring atoms of said second cyclic group have more than two adjacent atoms in common, i.e., said first and second cyclic groups share three or more common ring atoms. For example, the bicyclo[3.3.1]nonanyl group comprises two cyclohexane rings which have three adjacent carbon ring atoms in common.

Within the structural formulae described herein, any ring system (including any spiro, fused, or bridged ring system) may be connected to other parts of a molecule through any atom having suitable valency. For example, a bicyclic ring may be connected to another part of the molecule through a ring atom (e.g., a secondary carbon atom or heteroatom such as N), or a bridgehead (e.g., a tertiary carbon atom). Spiro, fused, and bridged rings may be fully unsaturated, partially unsaturated, or fully saturated, and may have aromatic character in one or more of their constituent rings.

As used herein, the term “cycloalkyl” means a saturated free radical having at least 3 to 9 carbon atoms (i.e., ring atoms) that form a ring. Exemplary cycloalkyl groups include cyclopropyl, cyclobutyl, cyclopentyl and cyclohexyl. It will be appreciated that the cycloalkyl group may be monocyclic or multicyclic (e.g., fused, bridged, or spirocyclic). In the case of multicyclic cycloalkyl groups, there are further rings, e.g. 1 or more further rings, all of which contain from 3 to 7 carbon atoms (i.e., ring atoms). Exemplary cycloalkyl groups having such further rings include bicyclo[1.1.1]pentanyl. The term “(C₃-C₇)cycloalkyl” denotes that the cycloalkyl group contains from 3 to 7 carbon atoms in the ring portion of the group, which may be monocyclic or multicyclic (e.g., fused, bridged, or spirocyclic), for example cyclopropanyl (having 3 ring carbon atoms) or bicyclo[1.1.1]pentanyl (having 5 ring carbon atoms). One or more ring atoms of the cycloalkyl group may be substituted by (i.e., bonded to) a heteroatom by a double bond (e.g., cycloalkyl substituted by oxo). The presence of such a substituent does not prevent the carbon backbone of the free radical being considered as a cycloalkyl group.

As used herein, the term “cycloalkenyl” means an unsaturated (i.e., partially or fully unsaturated) free radical having at least 3 to 9 carbon atoms (i.e., ring atoms) that form a ring. The term “cycloalkenyl” is not intended to encompass cyclic groups having aromatic character (those being considered as aryl groups as defined herein). Exemplary cycloalkenyl groups include cyclohexenyl. It will be appreciated that the cycloalkenyl group may be monocyclic or multicyclic (e.g., bridged). In the case of multicyclic cycloalkenyl groups, there are further rings, e.g. 1 or more further rings, all of which contain from 3 to 9 carbon atoms (i.e., ring atoms). Those further rings may be saturated or unsaturated. Exemplary cycloalkenyl groups having such further rings include bicyclo[2.2.1]hept-5-enyl. The term “(C₄-C₈)cycloalkenyl” denotes that the cycloalkenyl group contains from 4 to 8 carbon atoms in the ring portion of the group, for example cyclohexenyl (having 6 ring carbon atoms) or bicyclo[2.2.1]hept-5-enyl (having 7 ring carbon atoms). The double bond (or bonds) within the cycloalkenyl group is/are typically between ring carbon atoms (i.e., endocyclic), although may also be between one ring carbon atom and an adjacent non-ring carbon atom (i.e., exocyclic).

As used herein, the term “aryl” means an aromatic free radical having at least 6 carbon atoms (i.e., ring atoms) that form a ring. It will be appreciated that the aryl group may be monocyclic or multicyclic (e.g., fused). In the case of multicyclic aryl groups, there are further rings, e.g. 1 or more further rings, all of which contain at least 3 carbon atoms (i.e., ring atoms). The further rings may also contain one or more heteroatoms and they may be saturated, unsaturated, or aromatic. A multicyclic aryl group is typically attached to the rest of the molecule via an aromatic ring, and typically not via a ring containing a heteroatom. In embodiments, the multicyclic aryl group does not contain any ring heteroatoms. Examples of aryl groups include phenyl and naphthalenyl, as well as indenyl and indanyl groups. Other aryl groups include, for example, tetrahydroisoquinolinyl bonded to the rest of the molecule via its phenyl ring. The term “(C₆-C₁₀)aryl” denotes that the aryl group contains from 6 to 10 carbon atoms in the ring portion of the group, which may be monocyclic or multicyclic (e.g., fused), for example phenyl (having 6 ring carbon atoms) or indanyl (having 9 ring carbon atoms).

As used herein, the term “heterocycloalkyl” means a saturated free radical having at least 3 to atoms (i.e., ring atoms) that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen. For example, the term “4- to 10-membered heterocycloalkyl” means a saturated free radical containing from 4 to 10 ring atoms, of which one or more is a hetero ring atom. Heterocycloalkyl rings may have oxo substituents, typically adjacent to a heteroatom (e.g., 2-oxopyrrolidinyl), but the oxygen atom does not form part of the ring and is excluded from the number of ring atoms. The presence of such a substituent does not prevent the ring (or rings) of the free radical being considered as a heterocycloalkyl group. Exemplary heterocycloalkyl groups include tetrahydrofuranyl, piperidinyl, morpholinyl and piperazinyl. Any ring sulphur atom may optionally carry one or more pendant (i.e., non-ring) oxygen atoms, as found in, e.g., a sulfolanyl group. In the case of multicyclic heterocyclic groups, there are further rings, e.g. 1 or more further rings, all of which contain from 3 to 7 ring atoms selected from carbon, nitrogen, sulphur, and oxygen. The further rings may be saturated, or partially or fully unsaturated (e.g., having aromatic character). Multicyclic heterocyclic groups include fused, bridged and spirocyclic ring systems. Where a multicyclic heterocycloalkyl group contains an unsaturated fused ring, the group is typically not bonded to the rest of the molecule via that fused ring. Exemplary heterocyclic groups having such further rings include 2-oxaspiro[3.3]heptanyl, tetrahydroisoquinolinyl, 1-azaspiro[3.3]heptan-2-onyl, and 2-azabicyclo[4.1.0]heptanyl. Where a heterocycloalkyl group is described as being “X- to Y-membered” (where X and Y are integers), this means that the heterocycloalkyl group contains a total number of ring atoms from X to Y. Thus, for example, a “4- to 7-membered heterocycloalkyl group” contains a total of 4, 5, 6 or 7 ring atoms, for example tetrahydropyranyl (6 ring atoms).

As used herein, the term “heterocycloalkenyl” means an unsaturated (i.e., partially or fully unsaturated) free radical having at least 3 to 6 atoms (i.e., ring atoms) that form a ring, wherein at least 1 to 5 of said ring atoms are carbon and the remaining at least 1 to 5 ring atom(s) (i.e., hetero ring atom(s)) are selected independently from the group consisting of nitrogen, sulphur, and oxygen. Heterocycloalkenyl rings may have oxo substituents, typically adjacent to a heteroatom, but the oxygen atom does not form part of the ring and is excluded from the number of ring atoms. Exemplary heterocycloalkenyl groups include tetrahydropyridyl. Any ring sulphur atom may optionally carry one or more pendant (i.e., non-ring) oxygen atoms. It will be appreciated that the heterocycloalkenyl group may be monocyclic or multicyclic (e.g., bridged). In the case of multicyclic heterocycloalkenyl groups, there are further rings, e.g. 1 or more further rings, all of which contain from 3 to 6 ring atoms selected from carbon, nitrogen, sulphur, and oxygen. Said further rings may be saturated, or partially or fully unsaturated (e.g., having aromatic character). Multicyclic heterocycloalkenyl groups include fused, bridged and spirocyclic ring systems. Where a multicyclic heterocycloalkenyl group contains an unsaturated fused ring, the group is typically not bonded to the rest of the molecule via that fused ring. Exemplary heterocycloalkenyl groups having such further rings include tetrahydroindolyl. Where a heterocycloalkenyl group is described as being “X- to Y-membered”, this means that the heterocycloalkenyl group contains a total number of ring atoms from X to Y. Thus, for example, a “5- to 8-membered heterocycloalkenyl group” contains a total of 5, 6, 7 or 8 ring atoms, for example dihydropyranyl (6 ring atoms).

As used herein, the term “heteroaryl” means an aromatic (i.e., having aromatic character) free radical typically containing from 6 to 10 ring atoms, wherein 1 to 9 of said ring atoms are carbon and the remaining 1 to 9 ring atom(s) (i.e., hetero ring atom(s)) are selected independently from the group consisting of nitrogen, sulphur, and oxygen. It will be appreciated that the heteroaryl group may be monocyclic or multicyclic (e.g., fused). In the case of multicyclic heteroaryl groups, there are further rings, e.g. 1 or more further rings, all of which contain at least 3 atoms (i.e., ring atoms), which further rings may optionally be aromatic. Examples of heteroaryl groups include monocyclic groups such as pyridyl, and 2-oxopyridinyl, as well as multicyclic groups such as indolyl. Where a heteroaryl group is described as being “X- to Y-membered”, this means that the heteroaryl group contains a total number of ring atoms from X to Y. Thus, for example, a “5- to 10-membered heteroaryl group” contains a total of 5, 6, 7, 8, 9 or 10 ring atoms, for example indolyl (9 ring atoms).

As used herein, the terms “halo” and “halogen” mean fluorine, chlorine, bromine, or iodine.

These terms are used interchangeably and may refer to a halogen free radical group or to a halogen atom as such. Those of skill in the art will readily be able to ascertain the identification of which in view of the context in which this term is used in the present disclosure.

As used herein, the term “CN” means a free radical having a carbon atom linked to a nitrogen atom via a triple bond. The CN radical is attached via its carbon atom.

As used herein, the term “oxo” means a free radical wherein an oxygen atom is connected to the atom bearing this radical via a double bond. For example, where a carbon atom carries an oxo radical it forms a carbon-oxygen double bond. It will be appreciated that not all atoms within a given structure can be substituted by oxo, and that this will depend on the free valency of the atom to be substituted.

As used herein, “—C(O)—” means

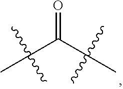 [SMILES: *C(*)=O]

“—S(O)₂—” means

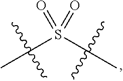 [SMILES: *S(*)(=O)=O]

“—C(O)NH—” means

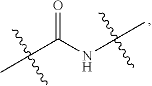 [SMILES: *NC(*)=O]

and “—NHC(O)—” means

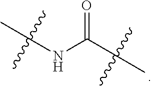 [SMILES: *NC(*)=O]

The compounds of the present disclosure are described, inter alia, by way of structural formulae. It will be appreciated that these formulae typically show only one form (e.g., resonance form, tautomeric form, etc.) of the compound, whereas certain compounds may exist in more than one such form. This will be readily apparent to the skilled reader. The present disclosure includes all possible tautomers of the compounds characterised by the structural formulae hereinbefore and below, including as single tautomers, or as any mixture of tautomers in any ratio. It will also be appreciated that certain of the present compounds may exist in one or more isomeric (e.g., stereoisomeric) forms. The present disclosure includes all possible stereoisomers, enantiomers, diastereomers, etc. of the compounds described hereinbefore and below, as well as cis- and trans-forms and conformers of the same. The purification and the separation of isomers may be accomplished by methods described hereinafter, as well as by techniques known in the art. For example, optical isomers of the compounds can be obtained by resolution of the racemic mixture of diastereoisomeric salts thereof (e.g., using an optically active acid or base, or by the formation of covalent diastereomers). A different process for separation of optical isomers involves the use of chiral chromatography (e.g., HPLC columns using a chiral phase), with or without conventional derivatization. Enzymatic separation, with or without derivatisation, may also be useful, and optically active compounds of the present disclosure can likewise be obtained by chiral syntheses utilizing optically active starting materials. The present disclosure includes all possible stereoisomers of the compounds described herein as single stereoisomers, or as any mixture of said stereoisomers, e.g. (R)- or (S)-isomers, in any ratio.

The compounds of the disclosure may exist in the form of free acids or bases, or may exist as addition salts with suitable acids or bases. For example, basic compounds of Formula (I) may be provided as pharmaceutically acceptable acid addition salts with an acid such as HCl, TFA, or formic acid (e.g., HCl). Methods for forming salts are described below and are also known in the art (see, e.g., Berge et al., J Pharm Sci. (1977) 66:1-19).

As used herein, the term “pharmaceutically acceptable” when used in connection with salts means a salt of a currently disclosed compound that may be administered without any resultant substantial undesirable biological effect(s) or any resultant deleterious interaction(s) with any other component of a pharmaceutical composition in which it may be contained.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Compositions and methods provided herein may be combined with one or more of any of the other compositions and methods provided herein.

The following abbreviations and empirical formulae are used herein:

| | |
|---|---|
| Ac | Acetyl |
| ADDP | 1,1′-(azodicarbonyl)dipiperidine |
| ATP | adenosine triphosphate |
| BOC/Boc | tert-butyloxy carbonyl |
| Bn | benzyl |
| BuLi | butyl lithium |
| CDI | 1,1′-carbonyldiimidazole |
| DAD | diode-array detection |
| DBU | 1,8-diazabicyclo[5.4.0]undec-7-ene |
| DCM | dichloromethane |
| DIEA/DIPEA | diisopropylethylamine |
| Diox | 1,4-dioxane |
| 4-DMAP | 4-dimethylaminopyridine |
| DMF | N,N-dimethylformamide |
| DMSO | dimethyl sulfoxide |
| 4EBP1 | eukaryotic translation initiation factor 4E- |
| | binding protein 1 |
| EDC/EDCI | 1-ethyl-3-(3- |
| | dimethylaminopropyl)carbodiimide |
| ee | enantiomeric excess |
| ERK | extracellular signal-regulated kinase |
| EtOAc/AcOEt | ethyl acetate |
| ES | electrospray ionisation |
| FRET | Forster resonance energy transfer |
| HATU | hexafluorophosphate azabenzotriazole |
| | tetramethyl uronium |
| IPA | isopropyl alcohol |
| Ir[dF(CF3)ppy]2(dtbpy))PF6 | [4,4′-Bis(1,1-dimethylethyl)-2,2′- |
| | bipyridine-N1,N1′]bis[3,5-difluoro-2-[5- |
| | (trifluoromethyl)-2-pyridinyl-N]phenyl- |
| | C]Iridium(III) hexafluorophosphate |
| LC/MS | liquid chromatography/mass spectrometry |
| LED | light-emitting diode |
| MAPK/MEK | mitogen-activated protein kinase |
| Me | methyl |
| MeCN/ACN | acetonitrile |
| MeOH | methanol |
| MeTHF | 2-methyltetrahydrofuran |
| MS | mass spectrometry |
| MTBE | methyl tert-butyl ether |
| Pd/C | palladium on carbon |
| PDA | photodiode array |
| Pd₂(dba)₃ | tris(dibenzylideneacetone)dipalladium(0) |
| Pd(dppf)Cl₂ | [1,1′- |
| | Bis(diphenylphosphino)ferrocene]di- |
| | chloropalladium(II) |
| Pd(PPh₃)₂Cl₂ | bis(triphenylphosphine) palladium(II) |
| | dichloride |
| PhOK | potassium phenolate |
| PMB | p-methoxybenzyl |
| PPh₃ | triphenylphosphine |
| Py | pyridine |
| rac | racemic mixture |
| RPMI | Roswell Park Memorial Institute medium |
| RT | room temperature |
| SCX | strong cation exchange |
| SFC | Supercritical Fluid Chromatography |
| TAD | transcriptional activation domain |
| TATU | O-(7-Azabenzotriazole-1-yl)-N,N,N′,N′- |
| | tetramethyluronium tetrafluoroborate |
| TBAF | tetrabutylammonium fluoride |
| TBTU | 2-(1H-Benzotriazole-1-yl)-1,1,3,3- |
| | tetramethylaminium tetrafluoroborate |
| TEA | triethylamine |
| TFA | trifluoroacetic acid |
| THF | tetrahydrofuran |
| TLC | thin layer chromatography |
| t_{RA} | retention time |
| uma | unified atomic mass unit |
| UV | ultraviolet |

### Compounds

In a first aspect the present disclosure provides a compound being of Formula (I)

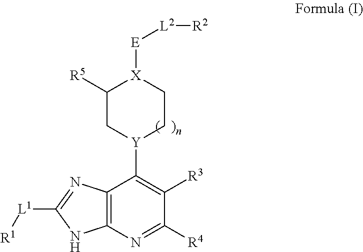

or a pharmaceutically acceptable salt thereof, wherein:
   R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
      wherein each R^{A} is independently selected from halo, —OH, —NHR′, —CN, oxo, —SO₂CH₃, —(C₁-C₃)alkyl, —O(C₁-C₃)alkyl, —C(O)R″, —C(O)NHR′, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, —NH₂, and —CN,
      and/or wherein two occurrences of R^{A} may be taken together with the atom(s) to which they are attached to form a (C₃-C₆)cycloalkyl group, or a 3- to 6-membered heterocycloalkyl group,
      wherein each R′ is independently selected from —H, methyl, and phenyl, and
      wherein each R″ is independently selected from —OH, methyl, phenyl optionally substituted by —OCF₃, and tetrahydrofuranyl;
   L¹ is selected from a direct bond, —O—, —CH₂—, and —CH═;
   R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
      wherein each R^{B} is selected from halo, —OH, oxo, —NH₂, —NHMe, —NO₂, —CN, —SF₅, —SiMe₃, —B(OH)₂, —(C₁-C₃)alkyl, —O(C₁-C₃)alkyl, —(C₃-C₆)cycloalkyl, —O(C₃—C₆)cycloalkyl, —SO₂CH₃, —NHC(O)Me, —NHC(O)OC(CH₃)₃, phenyl, benzyl, 1-λ⁶-2-thiazolidine-1,1-dionyl, and pyridyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
      and further wherein, when R² is —(C₃-C₆)cycloalkyl, R² is optionally substituted at one ring atom by a spirocyclic oxindolyl group;
   L² is selected from a direct bond, —CH₂—, —CH₂CH₂—, and —CH₂O—, wherein L² (when present) is optionally substituted with —NH₂ or methyl;
   R³ is selected from —H, halo, —OH, and methyl optionally substituted with —OH;
   R⁴ is —H, or —NH₂;
   R⁵ is —H, or methyl;
   X is CH or N;
   Y is selected from CH, CF, and N (with the proviso that when Y is N, X is also N);
   E is selected from —C(O)—, —S(O)₂—, and —O— (with the proviso that when E is —O—, X and Y are both CH); and
n is 0, 1, or 2.

In embodiments, the compound is a compound of Formula (I) or a pharmaceutically acceptable salt thereof, wherein:
R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
   wherein each R^{A} is independently selected from halo, —OH, —NHR′, —CN, oxo, —SO₂CH₃, —(C₁-C₃)alkyl, —O(C₁-C₃)alkyl, —C(O)R″, —C(O)NHR′, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, —NH₂, and —CN,
   and/or wherein two occurrences of R^{A} may be taken together with the atom(s) to which they are attached to form a (C₃-C₆)cycloalkyl group, or a 3- to 6-membered heterocycloalkyl group,
   wherein each R′ is independently selected from —H, methyl, and phenyl, and
   wherein each R″ is independently selected from methyl, phenyl optionally substituted by —OCF₃, and tetrahydrofuranyl;
L¹ is selected from a direct bond, —O—, —CH₂—, and —CH═;
R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
   wherein each R^{B} is selected from halo, —OH, —NH₂, —NO₂, —CN, —SF₅, —B(OH)₂, —(C₁-C₃)alkyl, —O(C₁-C₃)alkyl, —(C₃-C₆)cycloalkyl, —SO₂CH₃, phenyl, benzyl, 1-λ⁶-2-thiazolidine-1,1-dionyl, and pyridyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
   and further wherein, when R² is —(C₃-C₆)cycloalkyl, R² is optionally substituted at one ring atom by a spirocyclic oxindolyl group;
L² is selected from a direct bond, —CH₂—, —CH₂CH₂—, and —CH₂O—, wherein L² (when present) is optionally substituted with —NH₂ or methyl;
R³ is selected from —H, halo, —OH, and methyl optionally substituted with —OH;
R⁴ is —H, or —NH₂;
R⁵ is —H, or methyl;
X and Y are each independently selected from CH, and N (with the proviso that when Y is N, X is also N);
E is selected from —C(O)—, —S(O)₂—, and —O— (with the proviso that when E is —O—, X and Y are both CH); and
n is 0, 1, or 2.

In embodiments, the compound is a compound of Formula (I) or a pharmaceutically acceptable salt thereof, wherein:
R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
   wherein each R^{A} is independently selected from halo, —OH, —NHR′, —CN, oxo, —SO₂CH₃, —(C₁-C₃)alkyl, —O(C₁-C₃)alkyl, —C(O)R″, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, and —NH₂,
   and/or wherein two occurrences of R^{A} may be taken together with the atom(s) to which they are attached to form a (C₃-C₆)cycloalkyl group, or a 3- to 6-membered heterocycloalkyl group,
   wherein each R′ is independently selected from —H, and methyl, and
   wherein each R″ is independently selected from —OH, methyl, and tetrahydrofuranyl;
L¹ is selected from a direct bond, and —CH₂—;
R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
   wherein each R^{B} is selected from halo, —OH, oxo, —NH₂, —NHMe, —NO₂, —CN, —SF₅, —SiMe₃, —(C₁-C₃)alkyl, —O(C₁-C₃)alkyl, —(C₃-C₆)cycloalkyl, —O(C₃-C₆)cycloalkyl, —SO₂CH₃, —NHC(O)Me, —NHC(O)OC(CH₃)₃, phenyl, benzyl, and 1-λ⁶-2-thiazolidine-1,1-dionyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
   and further wherein, when R² is —(C₃-C₆)cycloalkyl, R² is optionally substituted at one ring atom by a spirocyclic oxindolyl group;
L² is selected from a direct bond, —CH₂—, and —CH₂O—, wherein L² (when present) is optionally substituted with methyl;
R³ is selected from —H, and halo;
R⁴ is —H;
R⁵ is —H, or methyl;
X is CH, or N;
Y is CH, or CF;
E is selected from —C(O)—, and —S(O)₂—; and
n is 0, 1, or 2.

In embodiments, the compound is a compound of Formula (I) or a pharmaceutically acceptable salt thereof, wherein:
R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
   wherein each R^{A} is independently selected from halo, —OH, —NHR′, —CN, oxo, —SO₂CH₃, —(C₁-C₃)alkyl, —O(C₁-C₃)alkyl, —C(O)R″, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, and —NH₂,
   and/or wherein two occurrences of R^{A} may be taken together with the atom(s) to which they are attached to form a (C₃-C₆)cycloalkyl group, or a 3- to 6-membered heterocycloalkyl group,
   wherein each R′ is independently selected from —H, and methyl, and
   wherein each R″ is independently selected from methyl, and tetrahydrofuranyl;
L¹ is selected from a direct bond, and —CH₂—;
R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
   wherein each R^{B} is selected from halo, —OH, —NH₂, —NO₂, —CN, —SF₅, —(C₁-C₃)alkyl, —O(C₁-C₃)alkyl, —(C₃-C₆)cycloalkyl, —SO₂CH₃, phenyl, benzyl, and 1-λ⁶-2-thiazolidine-1,1-dionyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
   and further wherein, when R² is —(C₃-C₆)cycloalkyl, R² is optionally substituted at one ring atom by a spirocyclic oxindolyl group;
L² is selected from a direct bond, —CH₂—, and —CH₂O—, wherein L² (when present) is optionally substituted with methyl;
R³ is selected from —H, and halo;
R⁴ is —H;
R⁵ is —H, or methyl;
X and Y are each independently selected from CH, and N (with the proviso that Y is not N);
E is selected from —C(O)—, and —S(O)₂—; and
n is 0, 1, or 2.

In embodiments, the compound is a compound of Formula (I-A)

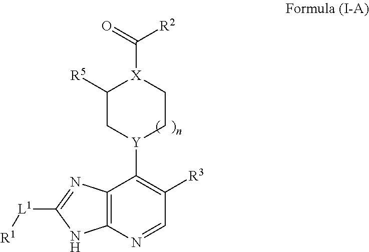

or a pharmaceutically acceptable salt thereof, wherein R¹, R², R³, R⁵, L¹, X, Y, and n are as defined herein.

In embodiments, the compound is a compound of Formula (I-B)

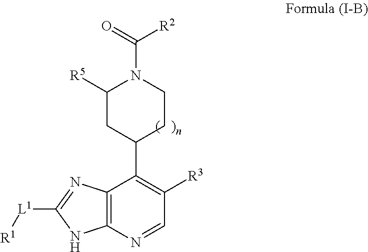

or a pharmaceutically acceptable salt thereof, wherein R¹, R², R³, R⁵, L¹, and n are as defined herein.

In embodiments, R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A}, wherein each R^{A} is independently selected from halo, —OH, —NHR′, —CN, oxo, —SO₂CH₃, —(C₁-C₃)alkyl, —O(C₁-C₃)alkyl, —C(O)R″, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, and —NH₂, and/or wherein two occurrences of R^{A} may be taken together with the atom(s) to which they are attached to form a (C₃-C₆)cycloalkyl group, or a 3- to 6-membered heterocycloalkyl group, wherein each R′ is independently selected from —H, and methyl, and wherein each R″ is independently selected from —OH, methyl, and tetrahydrofuranyl.

In embodiments, R¹ is selected from —H, —(C₁-C₃)alkyl, —(C₃-C₆)cycloalkyl, —(C₅-C₇)cycloalkenyl, —(C₆-C₁₀)aryl, 4- to 9-membered heterocycloalkyl, 6-membered heterocycloalkenyl, 5- to 6-membered heteroaryl, and 9- to 10-membered heteroaryl, wherein R¹ is optionally substituted with one or more occurrences of R^{A} as defined herein.

In embodiments, R¹ is 6- to 10-membered multicyclic (e.g., bicyclic) heterocycloalkyl or 7- to 10-membered multicyclic (e.g., bicyclic) heteroaryl, wherein R¹ is optionally substituted with one or more occurrences of R^{A} as defined herein.

In embodiments, R¹ is 6- to 10-membered multicyclic (e.g., bicyclic) heterocycloalkyl optionally substituted with one or more occurrences of R^{A} as defined herein. In embodiments, R¹ is 7- to 9-membered multicyclic (e.g., bicyclic) heterocycloalkyl optionally substituted with one or more occurrences of R^{A}. In embodiments, R¹ is 7- to 9-membered bicyclic heterocycloalkyl comprising one or two (e.g., two) hetero ring atoms selected from nitrogen, oxygen, and sulfur, wherein R¹ is optionally substituted with one or more occurrences of R^{A}.

In embodiments, R¹ is 7- to 10-membered multicyclic (e.g., bicyclic) heteroaryl optionally substituted with one or more occurrences of R^{A} as defined herein. In embodiments, R¹ is 7- to 10-membered bicyclic heteroaryl comprising one or two (e.g., two) hetero ring atoms selected from nitrogen, oxygen, and sulfur, wherein R¹ is optionally substituted with one or more occurrences of R^{A}. In embodiments, R¹ is 9- or 10-membered bicyclic heteroaryl comprising one or two (e.g., two) hetero ring atoms selected from nitrogen, and sulfur, wherein R¹ is optionally substituted with one or more occurrences of R^{A}. In embodiments, R¹ is 8- to 9-membered (e.g., 9-membered) multicyclic (e.g., bicyclic) heteroaryl optionally substituted with one or more occurrences of R^{A}. In embodiments, R¹ is an unsubstituted 9-membered bicyclic heteroaryl.

In embodiments, R¹ is fully saturated. In other embodiments, R¹ is partially or fully unsaturated (e.g., a partially or fully unsaturated cyclic group). In embodiments, R¹ is selected from —(C₄-C₈)cycloalkenyl, 5- to 8-membered heterocycloalkenyl, —(C₆-C₁₀)aryl, and 9- to 10-membered heteroaryl, wherein R¹ is optionally substituted with one or more occurrences of R^{A} as defined herein. In embodiments, R¹ is —(C₄-C₈)cycloalkenyl, or 5- to 8-membered heterocycloalkenyl, wherein R¹ is optionally substituted with one or more occurrences of R^{A}. In embodiments, R¹ is —(C₆-C₁₀)aryl, or 9- to 10-membered heteroaryl, wherein R¹ is optionally substituted with one or more occurrences of R^{A}. In embodiments, R¹ is 5- to 8-membered heterocycloalkenyl, or 9- to 10-membered heteroaryl, wherein R¹ is optionally substituted with one or more occurrences of R^{A}. In embodiments, R¹ is —(C₄-C₈)cycloalkenyl, or —(C₆-C₁₀)aryl, wherein R¹ is optionally substituted with one or more occurrences of R^{A}.

In embodiments, R¹ is selected from —(C₄-C₈)cycloalkenyl, —(C₆-C₁₀)aryl, 7- to 10-membered multicyclic (e.g., bicyclic) heterocycloalkyl, 5- to 8-membered heterocycloalkenyl, and 7- to 10-membered multicyclic (e.g., bicyclic) heteroaryl, wherein R¹ is optionally substituted with one or more occurrences of R^{A} as defined herein, or R¹ is —(C₁-C₆)alkyl substituted by one or more substituents selected from —OH, —NH₂, and —O(C₁-C₃)alkyl.

In embodiments, R¹ is selected from:

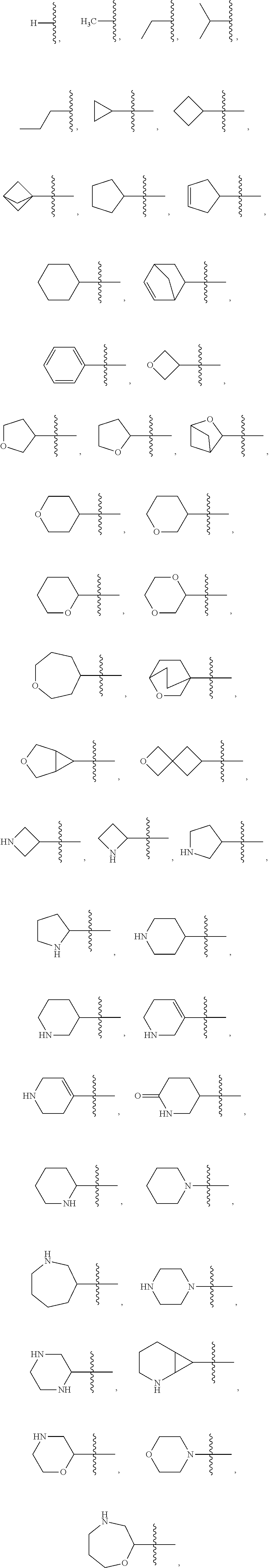

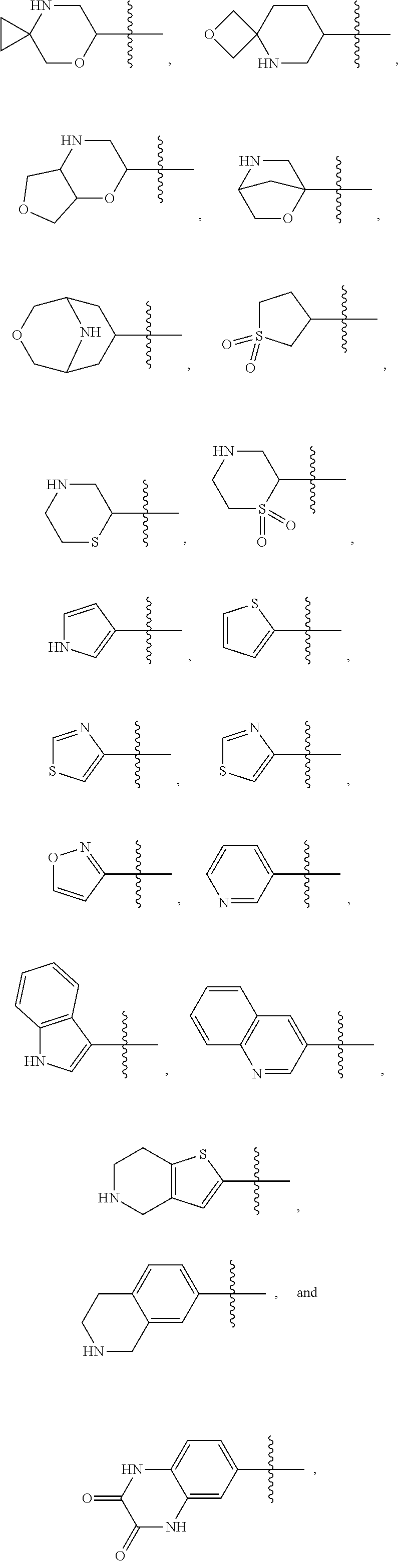

wherein R¹ is optionally substituted by one or more occurrences of R^{A} as defined herein.

In embodiments, R′ is selected from:

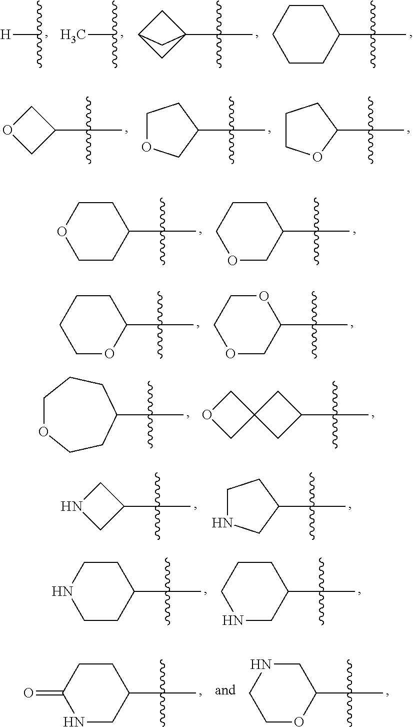

wherein R¹ is optionally substituted by one or more occurrences of R^{A} as defined hereinbefore. In embodiments, each R^{A} is independently selected from the group consisting of -halo, —OH, —NH₂, oxo, —(C₁-C₃)alkyl, —C(O)R″, and —O(C₁-C₃)alkyl, wherein each occurrence of (C₁-C₃)alkyl may be optionally substituted by one or more halo groups, wherein each R″ is independently selected from methyl, phenyl optionally substituted by —OCF₃, and tetrahydrofuranyl. In other embodiments (e.g., wherein R¹ is cycloalkyl) R¹ is substituted by two occurrences of R^{A} which, when taken together with the atom(s) to which they are attached, form a 3- to 6-membered heterocycloalkyl group.

In embodiments, R¹ is selected from:

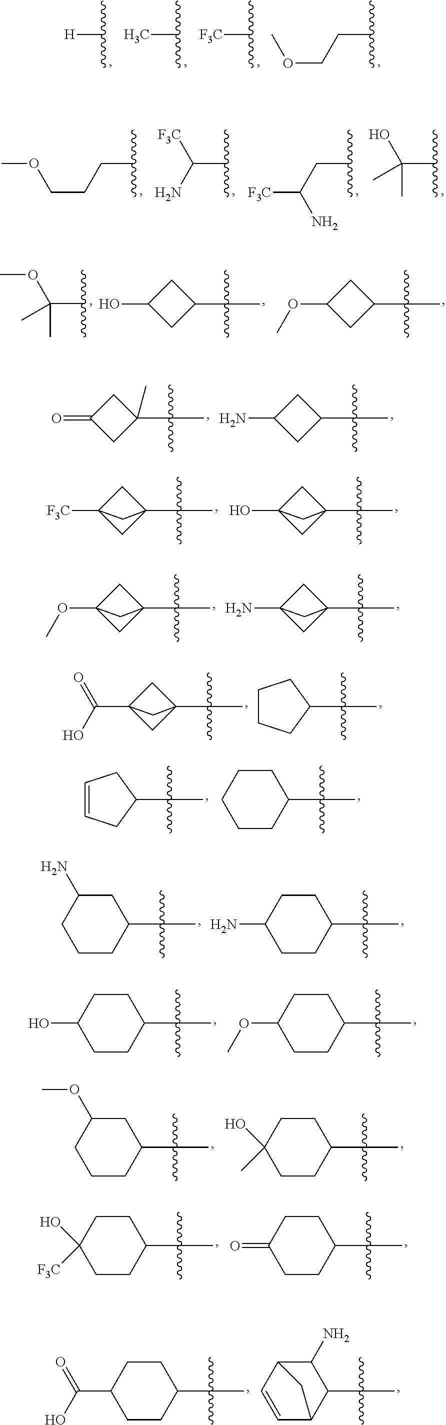

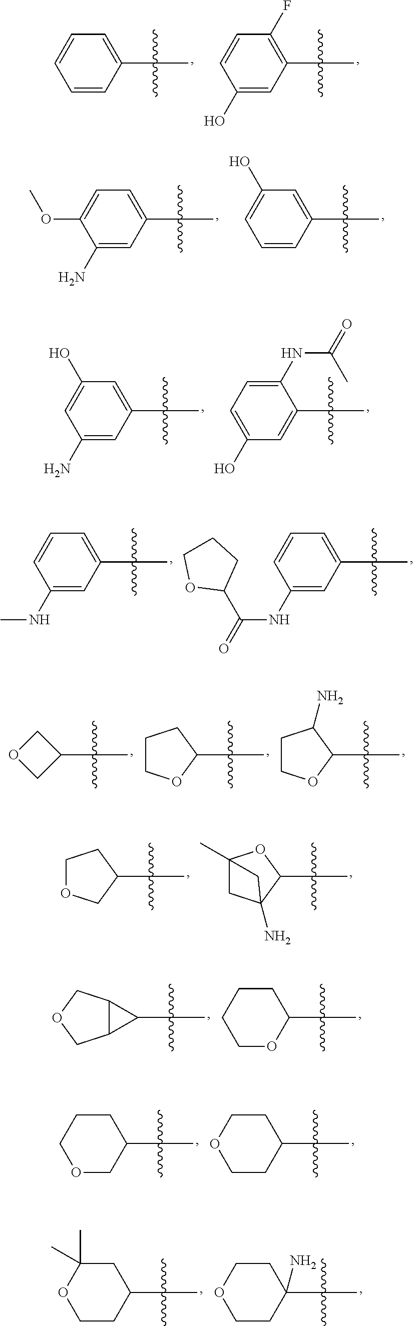

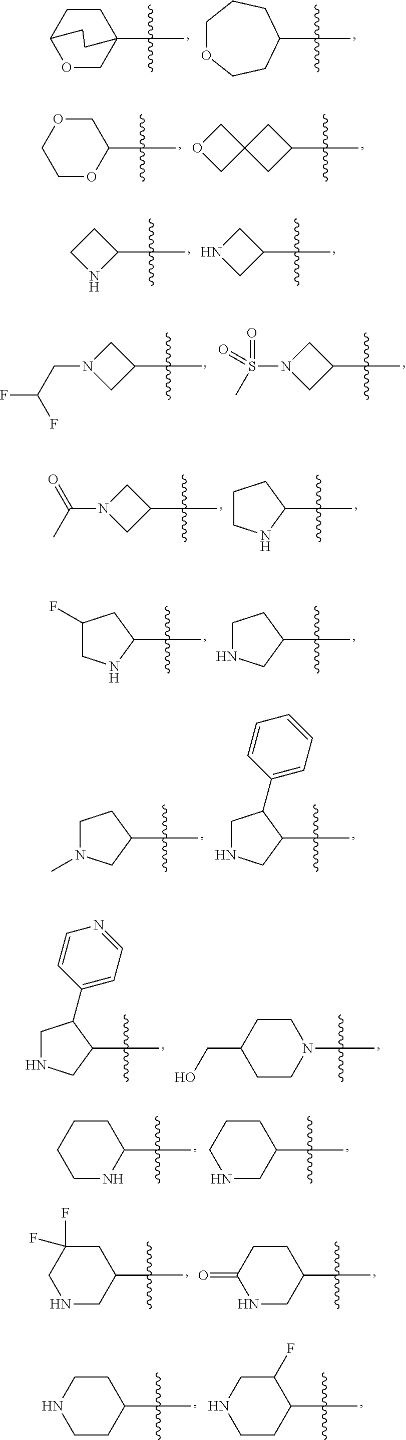

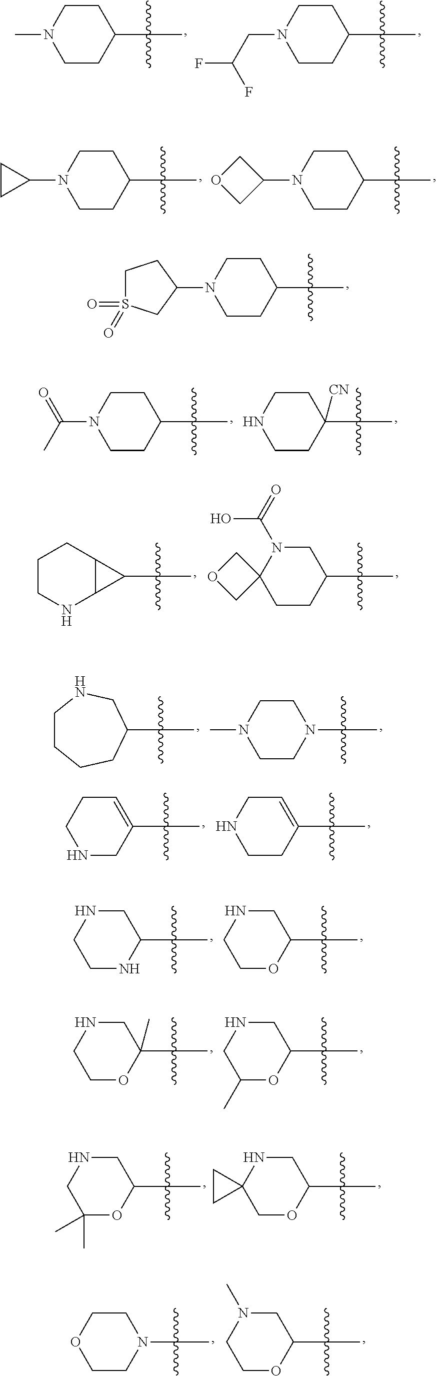

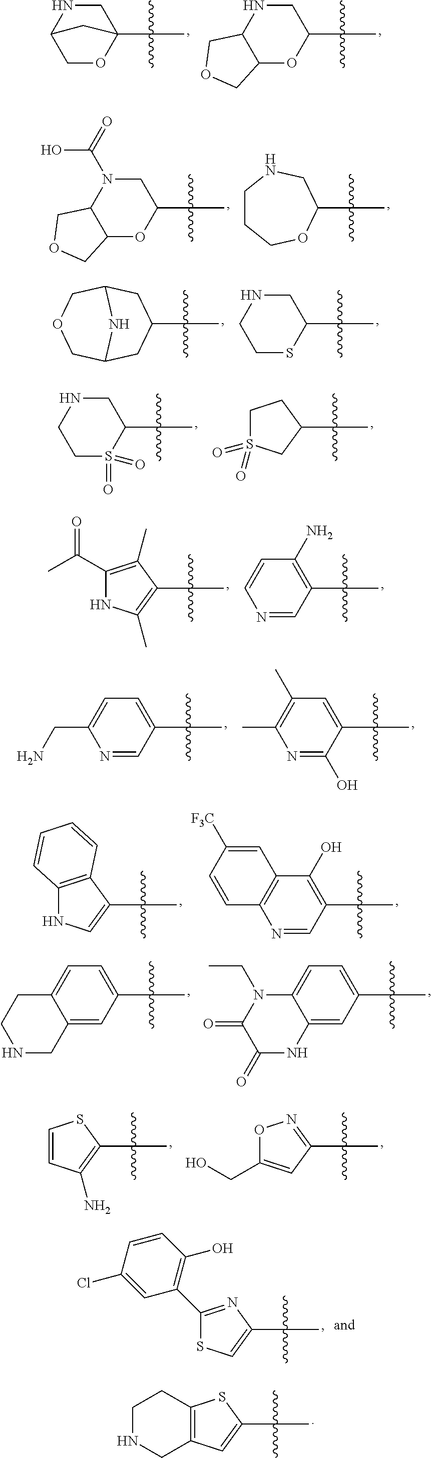

In embodiments, R¹ is selected from:

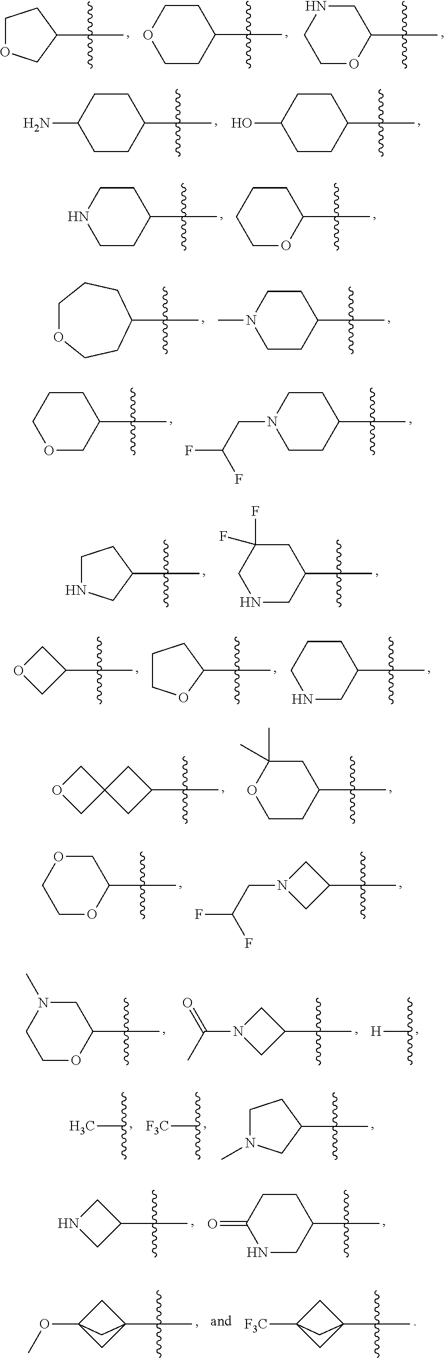

In embodiments, R¹ is —(C₄-C₆)cycloalkyl, or 5- to 7-membered heterocycloalkyl, wherein the heterocycloalkyl groups comprise one or two atoms independently selected from O and N, and wherein the cycloalkyl or heterocycloalkyl groups are optionally and independently substituted by one or more occurrences of R^{A} as defined hereinbefore. In embodiments, each R^{A} is independently selected from —OH, —NH₂, -Me, difluoroethyl, and —OCH₃.

In embodiments, R¹ is selected from:

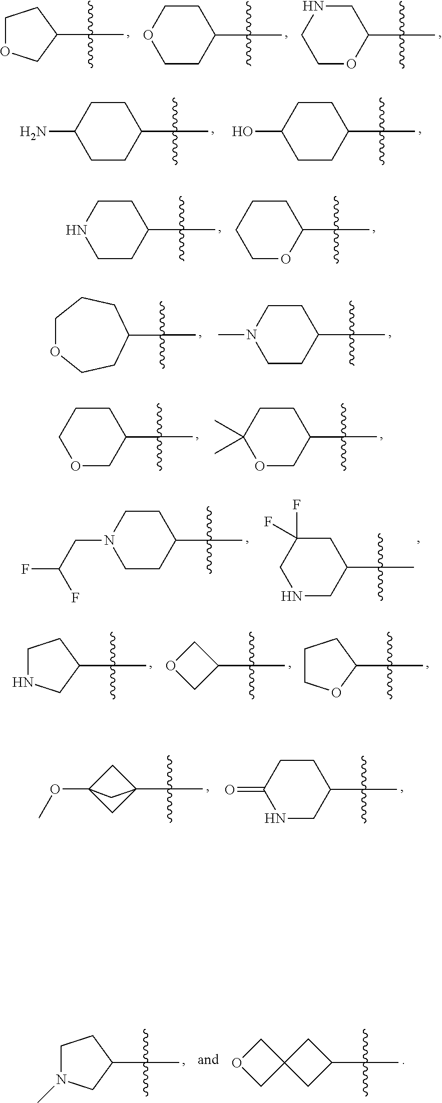

In other embodiments, R¹ is selected from:

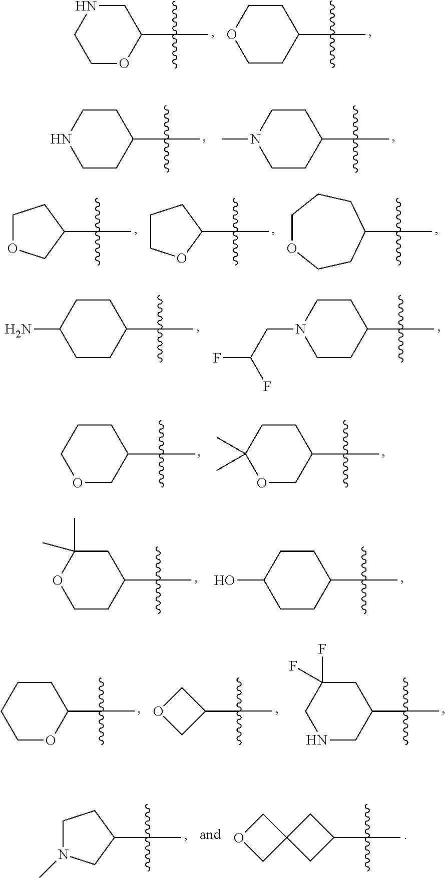

In embodiments, L¹ is a direct bond or —CH₂—. In embodiments, L¹ is a direct bond. In other embodiments, L¹ is —CH₂—.

In embodiments, L¹ is a direct bond or —CH₂—, and R¹ is —(C₄-C₆)cycloalkyl, or 5- to 7-membered heterocycloalkyl, wherein the heterocycloalkyl group comprises one or two atoms independently selected from O and N, and wherein the cycloalkyl or heterocycloalkyl group is optionally and independently substituted by one or more occurrences of R^{A} as defined hereinbefore. In embodiments, each R^{A} is independently selected from —OH, —NH₂, -Me, difluoroethyl, and —OCH₃.

In embodiments, L¹ is —CH₂— and R¹ is —(C₃-C₇)cycloalkyl, 4- to 10-membered heterocycloalkyl, or 5- to 10-membered heteroaryl, wherein R¹ is optionally substituted with one or more occurrences of R^{A} as defined herein.

In embodiments, when L¹ is —O—, R¹ is not —H.

In embodiments, R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B}, wherein each R^{B} is selected from halo, —OH, oxo, —NH₂, —NHMe, —NO₂, —CN, —SF₅, —SiMe₃, —(C₁-C₃)alkyl, —O(C₁-C₃)alkyl, —(C₃-C₆)cycloalkyl, —O(C₃-C₆)cycloalkyl, —SO₂CH₃, —NHC(O)Me, —NHC(O)OC(CH₃)₃, phenyl, benzyl, and 1-λ⁶-2-thiazolidine-1,1-dionyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃, and further wherein, when R² is —(C₃-C₆)cycloalkyl, R² is optionally substituted at one ring atom by a spirocyclic oxindolyl group.

In embodiments, R² is selected from —(C₃-C₆)cycloalkyl, 5- to 6-membered heterocycloalkyl, 9- to 10-membered heterocycloalkyl, —(C₆-C₁₀)aryl, 5- to 6-membered heteroaryl, and 9- to 10-membered heteroaryl, wherein R² is optionally substituted with one or more occurrences of R^{B} as defined herein.

In embodiments, R² is selected from —(C₄-C₆)cycloalkyl, —(C₆-C₁₀)aryl, and 5- to 10-membered heteroaryl, wherein R² is optionally substituted by one or more occurrences of R^{B} as defined hereinbefore. In embodiments, each R^{B} is independently selected from the group consisting of halo, —NO₂, —NH₂, —OCH₃, —OCF₃, —SF₅, —(C₁-C₃)alkyl optionally substituted by one or more halo, and —(C₃-C₆)cycloalkyl optionally substituted by —CF₃.

In embodiments, R² is selected from —(C₅-C₆)cycloalkyl, phenyl, and 6- to 9-membered heteroaryl, wherein R² is optionally substituted by one or more occurrences of R^{B}, wherein each R^{B} is independently selected from the group consisting of halo, —NO₂, —NH₂, —OCH₃, —OCF₃, —SF₅, —(C₁-C₂)alkyl optionally substituted by one or more fluoro, and cyclopropyl optionally substituted by —CF₃.

In embodiments, R² is selected from:

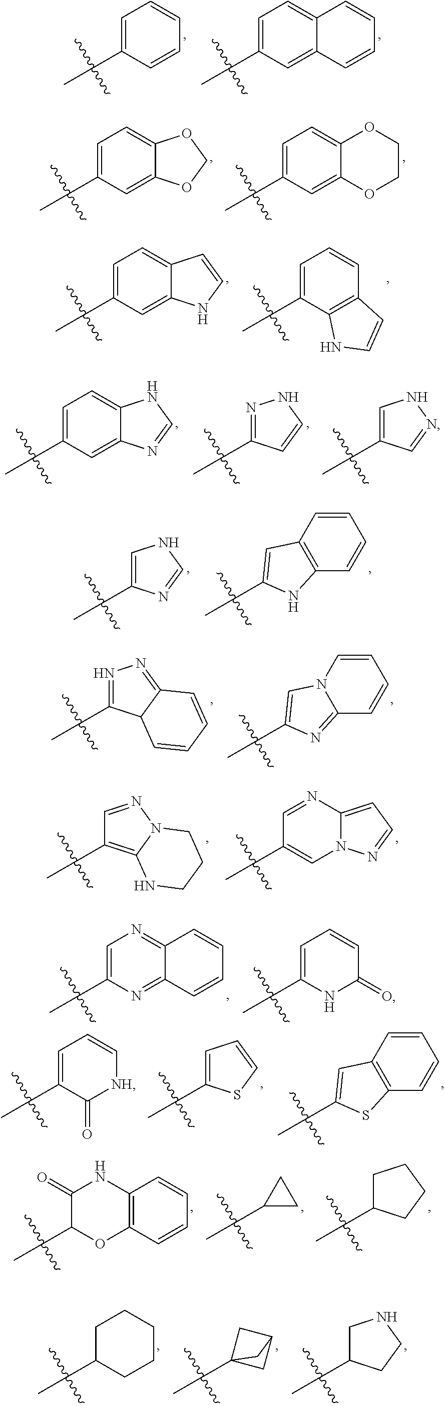

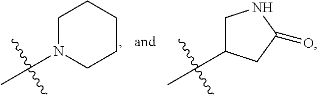

wherein R² is optionally substituted by one or more occurrences of R^{B} as defined herein.

In embodiments, R² is —(C₃-C₆)cycloalkyl optionally substituted by one or more occurrences of R^{B} as defined herein, further wherein R² is optionally substituted at one ring atom by a spirocyclic oxindolyl group.

In embodiments, R^{B} is selected from —NO₂, —SF₅, —SiMe₃, —B(OH)₂, —(C₃-C₆)cycloalkyl, —O(C₃-C₆)cycloalkyl, phenyl, and benzyl optionally substituted by one or more groups independently selected from halo, —OH, —NH₂, and —CF₃, wherein each occurrence of —(C₃-C₆)cycloalkyl, and phenyl, is substituted by one or more groups independently selected from halo, —OH, —NH₂, and —CF₃. In embodiments, R^{B} is not —O(C₃-C₆)cycloalkyl. In embodiments, R^{B} is not —O-cyclopropyl. In embodiments, R^{B} is not —NHC(O)OC(CH₃)₃.

In embodiments, at least one occurrence of R^{B} is —SF₅. In embodiments, one occurrence of R^{B} is —SF₅. In embodiments, R² is substituted with one occurrence of R^{B}, and R^{B} is —SF₅. In embodiments, R² is substituted with two occurrences of R^{B}, wherein one occurrence of R^{B} is —SF₅, and the other occurrence of R^{B} is selected from —F, —OH, —NH₂, —NHMe, and —NO₂. In embodiments, R² is substituted with two occurrences of R^{B}, wherein one occurrence of R^{B} is —SF₅, and the other occurrence of R^{B} is selected from —OH and —NH₂. In embodiments, R² is substituted with two occurrences of R^{B}, wherein one occurrence of R^{B} is —SF₅, and the other occurrence of R^{B} is —NH₂.

In embodiments, R² is phenyl substituted with one, or two occurrences of R^{B}, and at least one occurrence of R^{B} is —SF₅. In embodiments, R² is phenyl substituted with one, or two occurrences of R^{B}, and one occurrence of R^{B} is —SF₅. In embodiments, R² is phenyl substituted with one, or two occurrences of R^{B}, wherein one occurrence of R^{B} is —SF₅, and the other occurrence of R^{B} (where present) is selected from —F, —OH, —NH₂, —NHMe, and —NO₂. In embodiments, R² is phenyl substituted with one, or two occurrences of R^{B}, wherein one occurrence of R^{B} is —SF₅, and the other occurrence of R^{B} (where present) is selected from —OH, and —NH₂. In embodiments, R² is phenyl substituted with two occurrences of R^{B}, wherein one occurrence of R^{B} is —SF₅, and the other occurrence of R^{B} is selected from —F, —OH, —NH₂, —NHMe, and —NO₂. In embodiments, R² is phenyl substituted with two occurrences of R^{B}, wherein one occurrence of R^{B} is —SF₅, and the other occurrence of R^{B} is selected from —OH and —NH₂. In embodiments, R² is phenyl substituted with two occurrences of R^{B}, wherein one occurrence of R^{B} is —SF₅, and the other occurrence of R^{B} is —NH₂. In embodiments, R² is phenyl substituted with one occurrence of R^{B}, and R^{B} is —SF₅.

In embodiments, R² is selected from:

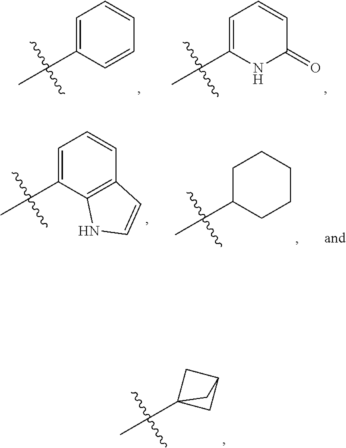

wherein R² is optionally substituted by one or more occurrences of R^{B} as defined hereinbefore. In embodiments, each R^{B} is independently selected from the group consisting of halo, —NH₂, —O(C₁-C₃)alkyl, —(C₁-C₃)alkyl, —(C₃-C₆)cycloalkyl, —SF₅, and —NO₂, wherein each occurrence of —O(C₁-C₃)alkyl, —(C₁-C₃)alkyl, and —(C₃-C₆)cycloalkyl, is optionally substituted by one or more groups independently selected from halo and —CF₃.

In embodiments, R² is selected from:

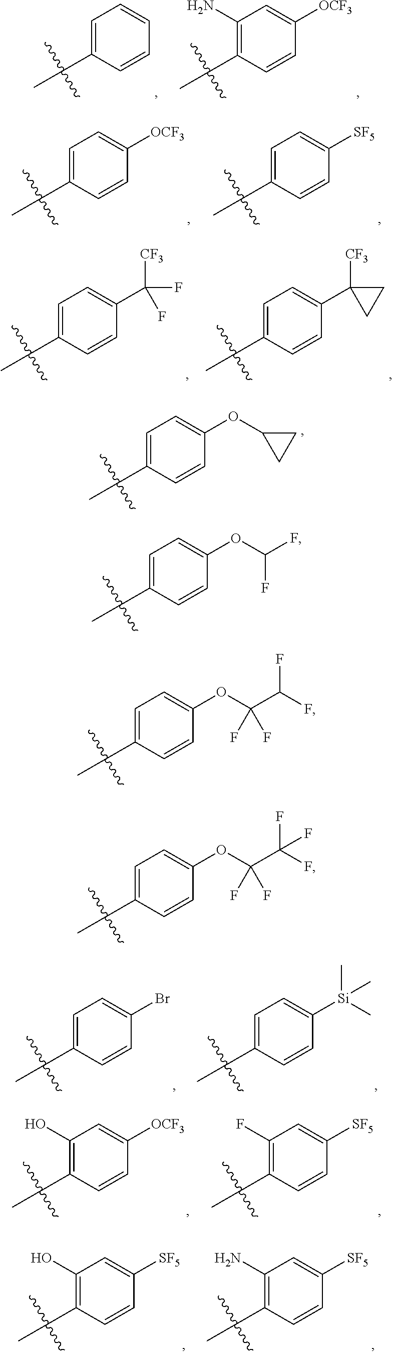

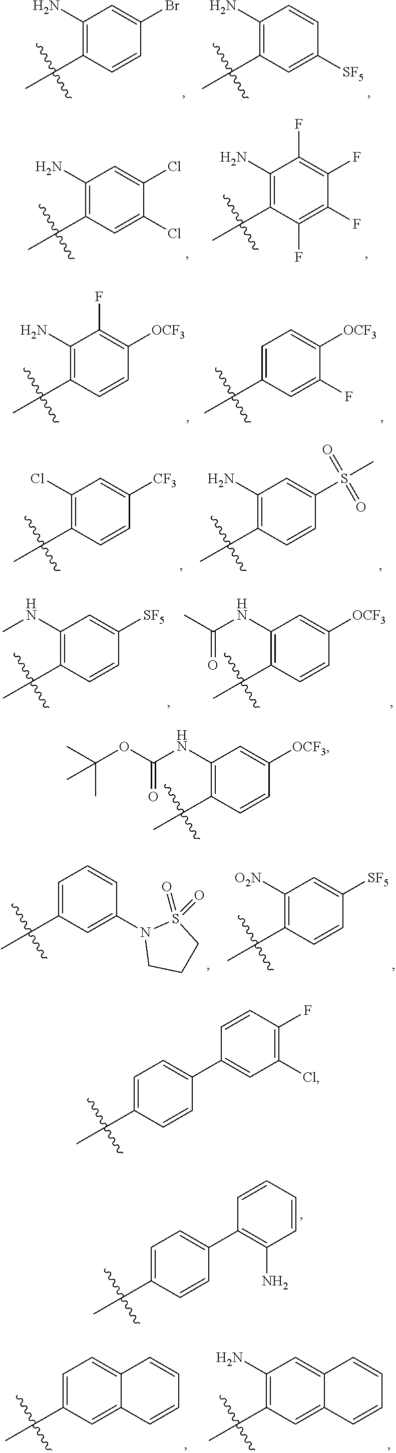

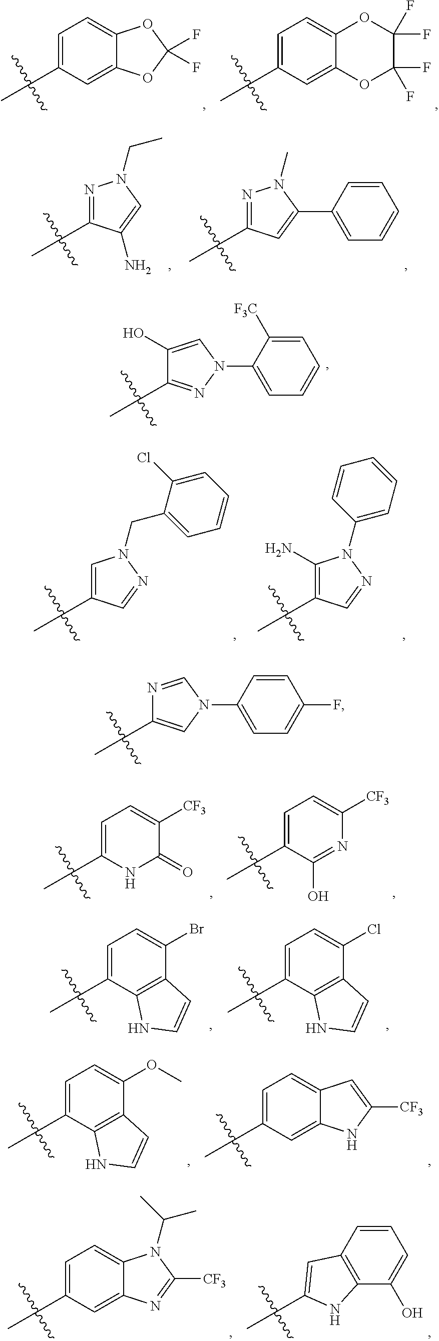

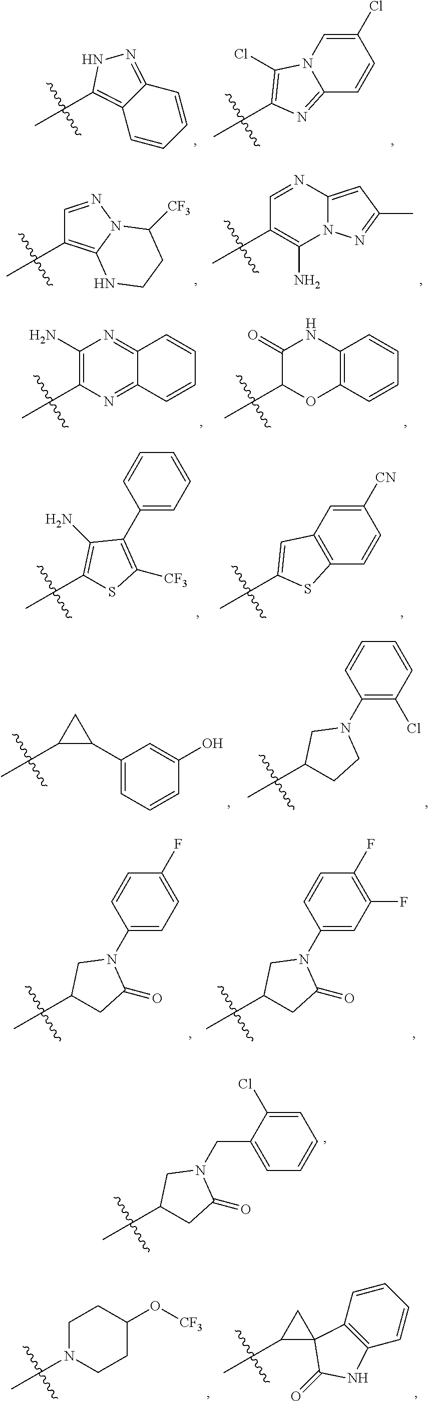

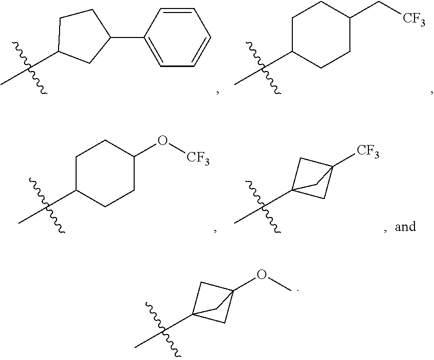

In embodiments, R² is selected from:

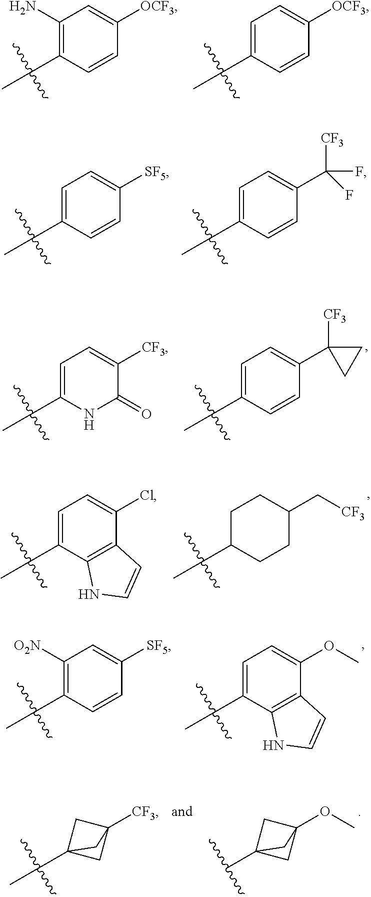

In embodiments, R² is selected from:

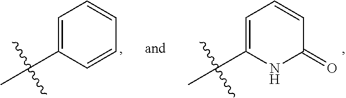

wherein R² is optionally substituted by one or more occurrences of R^{B}, wherein each R^{B} is independently selected from the group consisting of —NH₂, —OCF₃, —SF₅, and (C₁-C₃)alkyl optionally substituted by one or more halo.

In embodiments, R² is selected from:

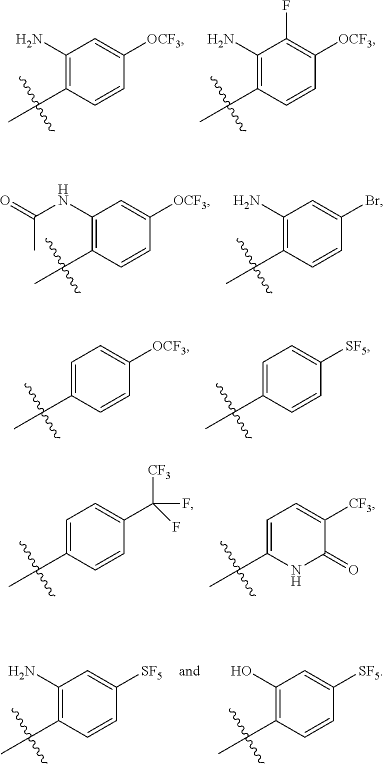

In embodiments, R² is selected from:

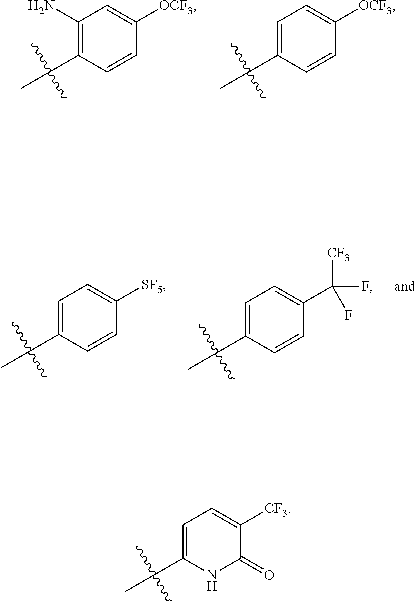

In embodiments, R² is

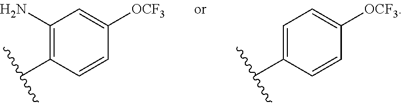 [SMILES: *c1ccc(OC(F)(F)F)cc1N ; *c1ccc(OC(F)(F)F)cc1]

In other embodiments, R² is

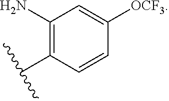 [SMILES: *c1ccc(OC(F)(F)F)cc1N]

In embodiments, R² is:

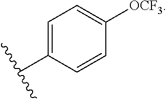 [SMILES: *c1ccc(OC(F)(F)F)cc1]

In embodiments, R² is:

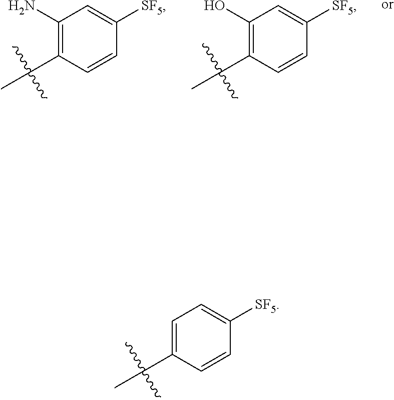

In embodiments, R² is

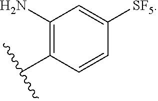

In other embodiments, R² is

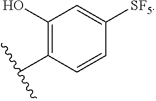

In other embodiments, R² is

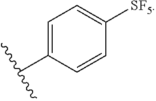

In embodiments, L² is selected from a direct bond, —CH₂—, and —CH₂O—, wherein L² (when present) is optionally substituted with methyl. In embodiments, L² is selected from a direct bond, unsubstituted —CH₂—, and —CH₂O— substituted with methyl.

In embodiments, L² is a direct bond.

In embodiments, L² is a direct bond, and R² is selected from:

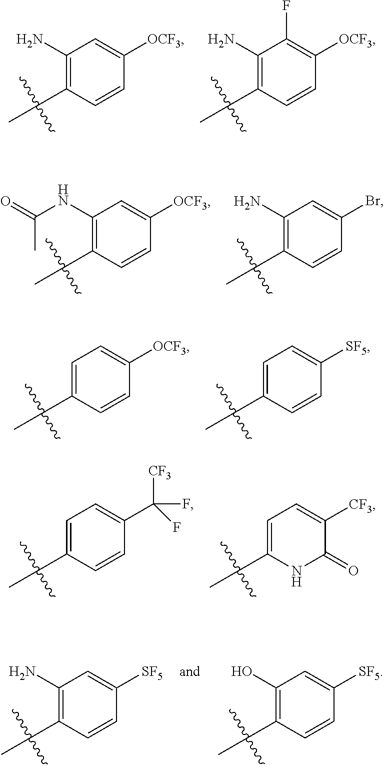

In embodiments, L² is a direct bond, and R² is selected from:

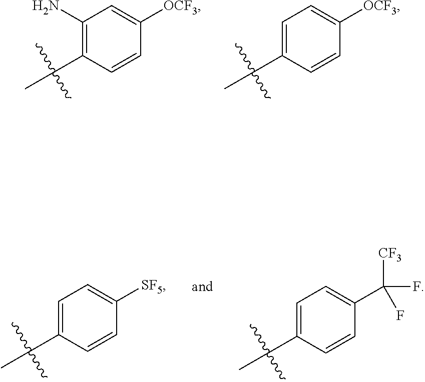

In embodiments, L² is a direct bond, and R² is selected from:

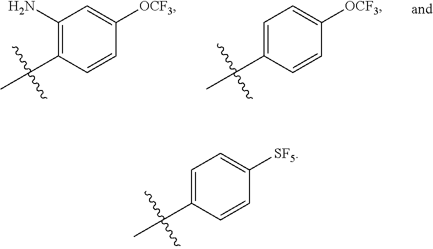

In embodiments, L² is a direct bond, and R² is selected from:

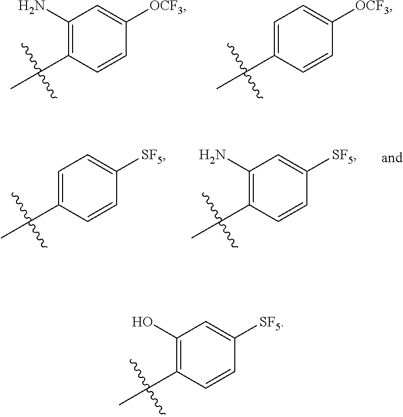

In embodiments, L² is a direct bond, and R² is:

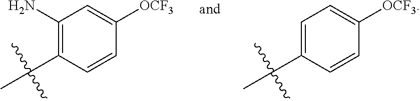 [SMILES: *c1ccc(OC(F)(F)F)cc1N ; *c1ccc(OC(F)(F)F)cc1]

In embodiments, L² is a direct bond, and R² is

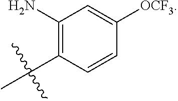 [SMILES: *c1ccc(OC(F)(F)F)cc1N]

In other embodiments, L² is a direct bond, and R² is

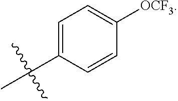 [SMILES: *c1ccc(OC(F)(F)F)cc1]

In embodiments, L² is a direct bond, and R² is:

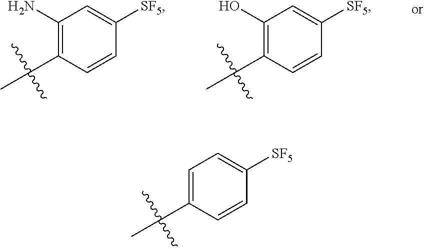

In embodiments, L² is a direct bond, and R² is

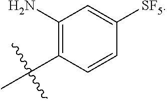

In other embodiments, L² is a direct bond, and R² is

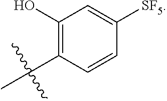

In other embodiments, L² is a direct bond, and R² is

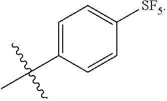

In embodiments, R³ is —H or halo (e.g., —F). In embodiments, R³ is halo (e.g., —F). In embodiments, R³ is —F.

In embodiments, R³ is —H. In embodiments, R⁴ is —H. In embodiments, R³ and R⁴ are both —H.

In embodiments, R³ is —H or halo (e.g., —F), and R⁴ is —H. In embodiments, R³ is halo (e.g., —F), and R⁴ is —H. In embodiments, R³ is —F, and R⁴ is —H.

In embodiments, R⁵ is —H. In embodiments, R³, R⁴, and R⁵ are all —H.

In embodiments, R⁵ is methyl.

In embodiments, R³ is halo (e.g., —F), R⁴ is —H, and R⁵ is —H. In embodiments, R³ is halo (e.g., —F), R⁴ is —H, and R⁵ is methyl. In embodiments, R³ is —F, R⁴ is —H, and R⁵ is —H. In embodiments, R³ is —F, R⁴ is —H, and R⁵ is methyl.

In embodiments, X is N.

In embodiments, Y is CH.

In embodiments, Y is CF. In embodiments, X is N and Y is CF.

In embodiments, E is —C(O)— or —S(O)₂—. In embodiments, E is —C(O)—.

In embodiments, n is 1. In embodiments, n is 0 or 2. In embodiments, n is 0. In embodiments, n is 2.

In embodiments, X is N and Y is CH. In embodiments, X is N, Y is CH and n is 1. In embodiments, X is N, Y is CH, n is 1 and R⁵ is —H.

In embodiments, X is N and E is —C(O)—. In embodiments, X is N, Y is CH, and E is —C(O)—.

In embodiments, X is N, Y is CH, R⁵ is —H, n is 1, and E is —C(O)—.

In embodiments, X is N, Y is CH, and n is 0. In embodiments, X is N, Y is CH, n is 0, and R⁵ is —H. In embodiments, X is N, Y is CH, n is 0, E is —C(O)—, L² is a direct bond, R⁴ is —H, and R⁵ is —H. Viewed from this aspect, the disclosure provides a compound of Formula (I-C)

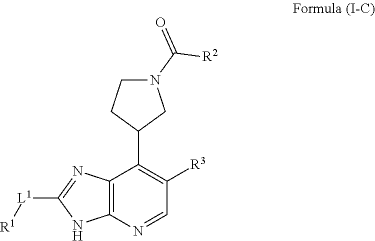

or a pharmaceutically acceptable salt thereof, wherein R¹, R², R³, and L¹ are as defined herein.

In embodiments, X is N, Y is CH, and n is 2. In embodiments, X is N, Y is CH, n is 2, and R⁵ is —H. In embodiments, X is N, Y is CH, n is 2, E is —C(O)—, L² is a direct bond, R⁴ is —H, and R⁵ is —H. Viewed from this aspect, the disclosure provides a compound of Formula (I-D)

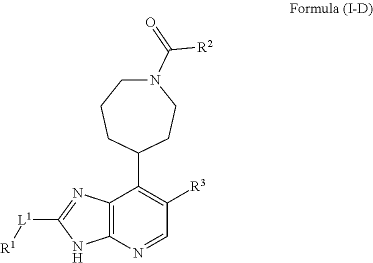

or a pharmaceutically acceptable salt thereof, wherein R¹, R², R³, and L¹ are as defined herein.

In embodiments, R¹ is cyclohexyl or 4- to 7-membered heterocycloalkyl, optionally substituted by one or more R^{A}, wherein: each R^{A} is independently selected from halo, and (C₁-C₃)alkyl optionally substituted by one or more halo; L¹ is a direct bond or —CH₂—; R² is

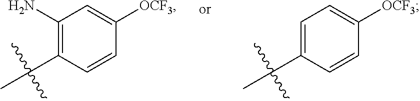 [SMILES: *c1ccc(OC(F)(F)F)cc1N ; *c1ccc(OC(F)(F)F)cc1]

L² is a direct bond; X is N, Y is CH, R³ is —H, R⁴ is —H, R⁵ is H, n is 1, and E is —C(O)—.

In embodiments, the compound is a compound of Formula (II)

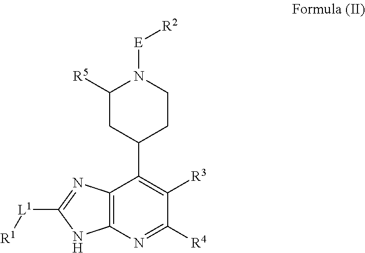

or a pharmaceutically acceptable salt thereof, wherein R¹, L¹, R³, R⁴, and R⁵ are as hereinbefore defined, and wherein:
   R² is selected from —(C₃-C₆)cycloalkyl (e.g., cyclopropyl), —(C₆-C₁₀)aryl, 5- to 10-membered heterocycloalkyl, and 5- to 10-membered heteroaryl, wherein R² is optionally substituted by one or more occurrences of R^{B}, wherein each R^{B} is independently selected from the group consisting of halo, —OH, oxo, —NO₂, —NH₂, —NHMe, —CN, —SF₅, —SO₂CH₃, —NHC(O)Me, —NHC(O)OC(CH₃)₃, —SiMe₃, —(C₁-C₃)alkyl optionally substituted by one or more halo, —O(C₁-C₃)alkyl optionally substituted by one or more halo, —(C₃-C₆)cycloalkyl optionally substituted by —CF₃, —O-cyclopropyl, phenyl optionally substituted by one or more halo, —OH, or —CF₃, benzyl optionally substituted by halo, and 1-λ⁶-2-thiazolidine-1,1-dionyl,
      and further wherein, when R² is cyclopropyl, R² is optionally substituted at one ring atom by a spirocyclic oxindolyl group; and
   E is —C(O)— or —S(O)₂—.

In embodiments, R² is selected from —(C₄-C₆)cycloalkyl, —(C₆-C₁₀)aryl, and 5- to 10-membered heteroaryl, wherein R² is optionally substituted by one or more occurrences of R^{B} wherein each R^{B} is independently selected from the group consisting of halo, —NO₂, —NH₂, —OCH₃, —OCF₃, —SF₅, —(C₁-C₃)alkyl optionally substituted by one or more halo, and —(C₃-C₆)cycloalkyl optionally substituted by —CF₃.

In embodiments, E is —C(O)—.

In embodiments, the compound is a compound of Formula (III)

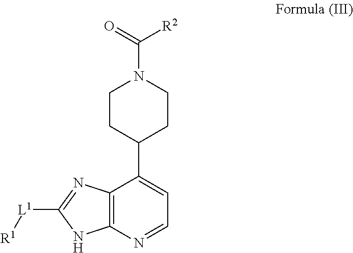

or a pharmaceutically acceptable salt thereof, wherein:
   R¹ is —H, —(C₁-C₃)alkyl, —(C₄-C₆)cycloalkyl, —(C₅-C₇)cycloalkenyl, —(C₆-C₁₀)aryl, or 4- to 9-membered heterocycloalkyl, 6-membered heterocycloalkenyl, 5- to 6-membered heteroaryl, and 9- to 10-membered heteroaryl, wherein R¹ is optionally substituted by one or more R^{A}, wherein each R^{A} is independently selected from halo, —OH, oxo, —NH₂, —NHMe, —C(O)OH, —C(O)Me, —SO₂CH₃, —NHC(O)R′, —(C₁-C₃)alkyl, —O(C₁-C₃)alkyl, cyclopropyl, oxetanyl, sulfolanyl, phenyl, benzyl, and pyridyl, wherein each occurrence of —(C₁-C₃)alkyl, —O(C₁-C₃)alkyl, and phenyl is optionally substituted by one or more groups independently selected from halo, —OH, and —NH₂, wherein each R″ is independently selected from methyl, and tetrahydrofuranyl;
   L¹ is a direct bond or —CH₂—; and
   R² is selected from —(C₃-C₆)cycloalkyl, —(C₆-C₁₀)aryl, 5- to 6-membered heterocycloalkyl, and 5- to 10-membered heteroaryl, wherein R² is optionally substituted by one or more occurrences of R^{B}, wherein each R^{B} is independently selected from the group consisting of halo, —NO₂, —NH₂, —NHMe, —NHAc, —NHBoc, —O(C₁-C₃)alkyl, —SF₅, —SiMe₃, —(C₁-C₃)alkyl, —O-cyclopropyl, 1-λ⁶-2-thiazolidine-1,1-dionyl, phenyl, benzyl, and —(C₃-C₆)cycloalkyl optionally substituted by —CF₃, wherein each occurrence of —(C₁-C₃)alkyl, —O(C₁-C₃)alkyl, phenyl, and benzyl, is optionally substituted by one or more groups independently selected from halo, —NH₂, and —CF₃—, and further wherein, when R² is cyclopropyl, R² is optionally substituted at one ring atom by a spirocyclic oxindolyl group.

In embodiments, R² is selected from —(C₄-C₆)cycloalkyl, (C₆-C₁₀)aryl, and 5- to 10-membered heteroaryl, wherein R² is optionally substituted by one or more occurrences of R^{B}, wherein each R^{B} is independently selected from the group consisting of halo, —NO₂, —NH₂, —OCH₃, —OCF₃, —SF₅, (C₁-C₃)alkyl optionally substituted by one or more halo, and —(C₃-C₆)cycloalkyl optionally substituted by —CF₃.

In embodiments, the compound is a compound of Formula (III), or a pharmaceutically acceptable salt thereof, wherein:
R¹ is cyclohexyl or 4- to 7-membered heterocycloalkyl, optionally substituted by one or more R^{A}, wherein each R^{A} is independently selected from halo, and (C₁-C₃)alkyl optionally substituted by one or more halo groups;
L¹ is a direct bond or —CH₂—; and
R² is selected from —(C₄-C₆)cycloalkyl, (C₆-C₁₀)aryl, and 5- to 10-membered heteroaryl, wherein R² is optionally substituted by one or more occurrences of R^{B}, wherein each R^{B} is independently selected from the group consisting of halo, —NO₂, —NH₂, —OCH₃, —OCF₃, —SF₅, (C₁-C₃)alkyl optionally substituted by one or more halo, and —(C₃-C₆)cycloalkyl optionally substituted by —CF₃.

In embodiments, R² is selected from:

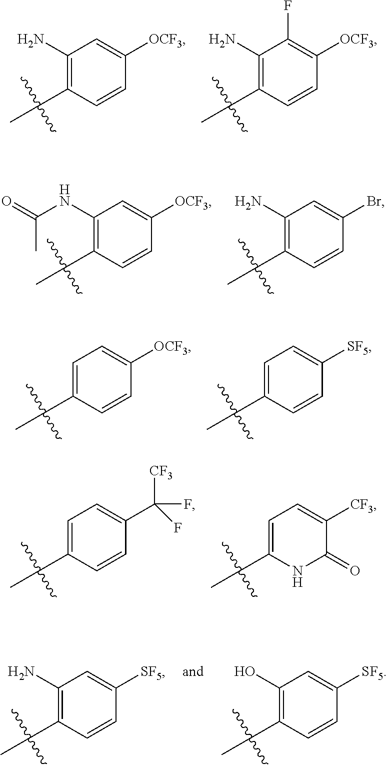

In embodiments, L² is a direct bond, and R² is selected from:

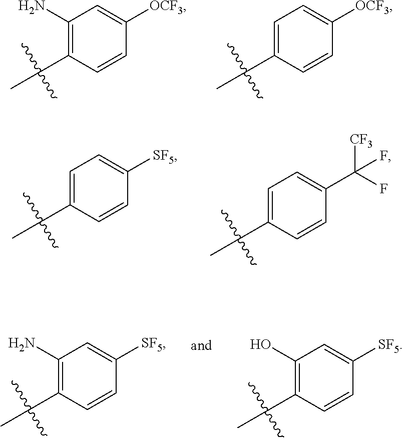

In embodiments, L² is a direct bond, and R² is selected from:

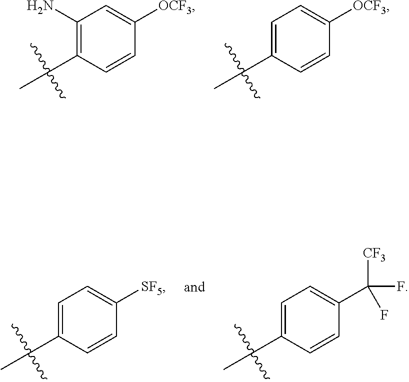

In other embodiments, L² is a direct bond, and R² is:

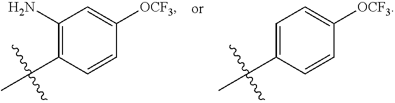 [SMILES: *c1ccc(OC(F)(F)F)cc1N ; *c1ccc(OC(F)(F)F)cc1]

In further embodiments, L² is a direct bond, and R² is

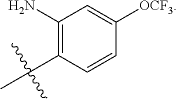 [SMILES: *c1ccc(OC(F)(F)F)cc1N]

In embodiments, R² is:

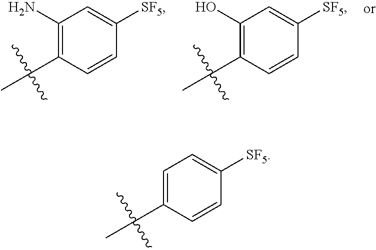

In embodiments, R² is

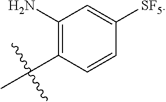

In embodiments, R² is

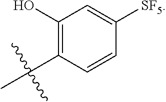

In other embodiments, R² is

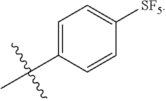

In embodiments, the compound is a compound of Formula (IV)

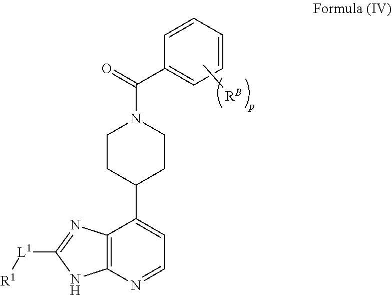

or a pharmaceutically acceptable salt thereof, wherein R¹, and L¹ are as hereinbefore defined, and wherein:
   p is 1, 2, 3, 4, or 5; and
   each R^{B} is independently selected from the group consisting of halo, —NO₂, —NH₂, —NHMe, —NHAc, —NHBoc, —OCH₃, —OCF₃, —SF₅, SiMe₃, (C₁-C₃)alkyl, —O(C₁-C₃)alkyl, —O— cyclopropyl, 1-λ⁶-2-thiazolidine-1,1-dionyl, phenyl, and —(C₃-C₆)cycloalkyl optionally substituted by —CF₃, wherein each occurrence of —(C₁-C₃)alkyl, —O(C₁-C₃)alkyl, and phenyl is optionally substituted by one or more groups independently selected from halo, —NH₂, and —CF₃.

In embodiments, p is 1 or 2; and each R^{B} is independently selected from the group consisting of halo, —NO₂, —NH₂, —OCH₃, —OCF₃, —SF₅, (C₁-C₃)alkyl optionally substituted by one or more halo, and —(C₃-C₆)cycloalkyl optionally substituted by —CF₃.

In embodiments, p is 1, 2, or 3. In embodiments, p is 1 or 2. In embodiments, p is 1. In other embodiments, p is 2. In other embodiments, p is 3.

In embodiments, p is 1 or 2, and each R^{B} is independently selected from halo, —OH, —OCF₃, —NH₂, —NHMe, —NHAc, and —SF₅. In embodiments, p is 1 or 2, and each R^{B} is independently selected from halo, —OH, —OCF₃, —NH₂, and —SF₅.

In embodiments, each R^{B} is independently selected from —OCF₃, —NH₂, and —SF₅, and p is 1 or 2.

In embodiments, p is 3, 4, or 5. In embodiments, p is 3, 4, or 5, and at least one R^{B} is halo (e.g., —F). In embodiments, p is 4 or 5, and 4 occurrences of R^{B} are halo (e.g., —F). In embodiments, p is 4 or 5, and 4 occurrences of R^{B} are —F.

In embodiments, the compound is a compound of Formula (V)

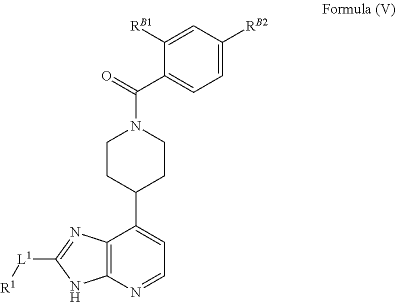

or a pharmaceutically acceptable salt thereof, wherein R¹, and L¹ are as hereinbefore defined, and wherein:
   R^{B1} is either —H, or is selected from the group consisting of halo (e.g. —F, or —Cl), —OH, —NH₂, —NHMe, —NHAc, —NHBoc, and —NO₂; and
   R^{B2} is selected from the group consisting of halo (e.g. —Br), —SF₅, —SO₂CH₃, —SiMe₃, —(C₁-C₃)alkyl optionally substituted by one or more halo groups, —O(C₁-C₃)alkyl optionally substituted by one or more halo groups, 1-trifluoromethylcyclopropan-1-yl, —O— cyclopropyl, and phenyl optionally substituted by one or more groups independently selected from halo, or —NH₂.

In embodiments, R^{B1} is either —H, or is selected from the group consisting of —NH₂, and —NO₂; and R^{B2} is selected from the group consisting of —OCF₃, —SF₅, —CF₂CF₃, —CH₂CF₃, and 1-trifluoromethylcyclopropan-1-yl.

In embodiments, R^{B1} is either —H, or is selected from the group consisting of halo (e.g., —F), —NH₂, —NHMe, —NHAc, and —OH; and R^{B2} is selected from the group consisting of —OCF₃ and —SF₅.

In embodiments, R^{B2} is —SF₅. In embodiments, R^{B1} is either —H, or is selected from the group consisting of halo (e.g., —F), —NH₂, —NHMe, —NO₂, and —OH; and R^{B2} is —SF₅. In embodiments, R^{B1} is either —H, or is selected from the group consisting of halo (e.g., —F), —NH₂, —NHMe, and —OH; and R^{B2} is —SF₅.

In embodiments, the compound is a compound of Formula (VI) or (VII)

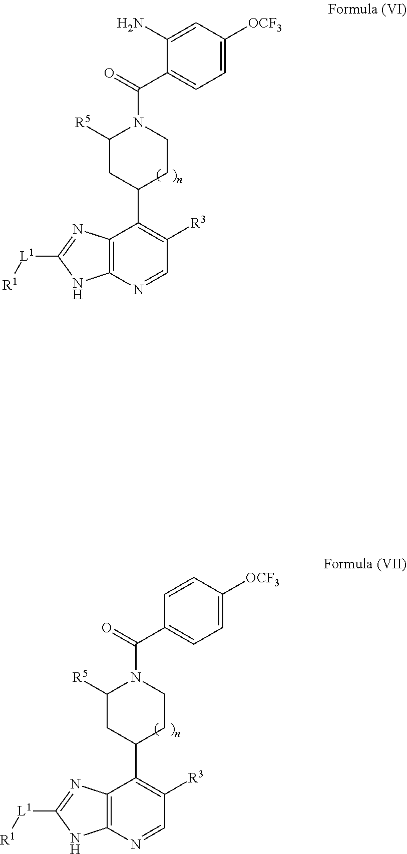

or a pharmaceutically acceptable salt thereof, wherein R¹, R³, R⁵, n, and L¹ are as hereinbefore defined.

In embodiments, the compound is a compound of Formula (VI), or a pharmaceutically acceptable salt thereof. In other embodiments, the compound is a compound of Formula (VII), or a pharmaceutically acceptable salt thereof.

In embodiments, the compound is a compound of Formula (VI-A) or (VII-A)

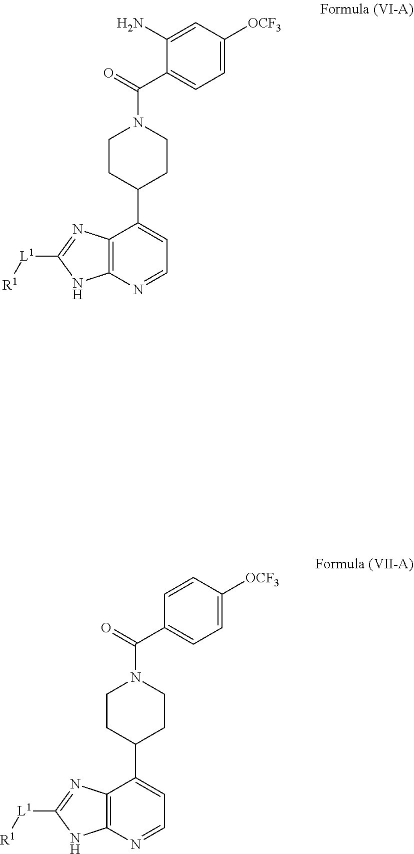

or a pharmaceutically acceptable salt thereof, wherein R¹ and L¹ are as hereinbefore defined.

In embodiments, L¹ is —CH₂—. In other embodiments, L¹ is a direct bond.

In embodiments, the compound is a compound of Formula (VI-A), or a pharmaceutically acceptable salt thereof. In other embodiments, the compound is a compound of Formula (VII-A), or a pharmaceutically acceptable salt thereof.

In embodiments, the compound is a compound of Formula (VIII), (IX), or (X)

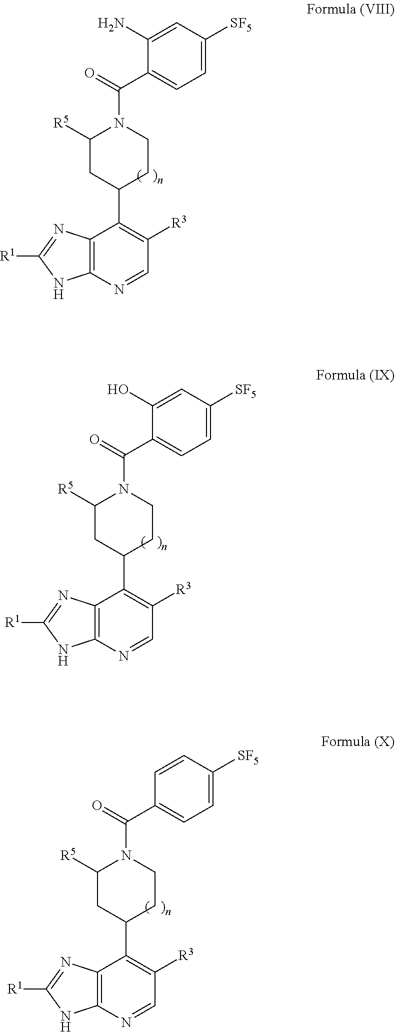

or a pharmaceutically acceptable salt thereof, wherein R¹, R³, R⁵, and n are as hereinbefore defined.

In embodiments, the compound is a compound of Formula (VIII), or a pharmaceutically acceptable salt thereof. In other embodiments, the compound is a compound of Formula (IX), or a pharmaceutically acceptable salt thereof. In other embodiments, the compound is a compound of Formula (X), or a pharmaceutically acceptable salt thereof.

In embodiments, the compound is a compound of Formula (VIII-A), (IX-A), or (X-A)

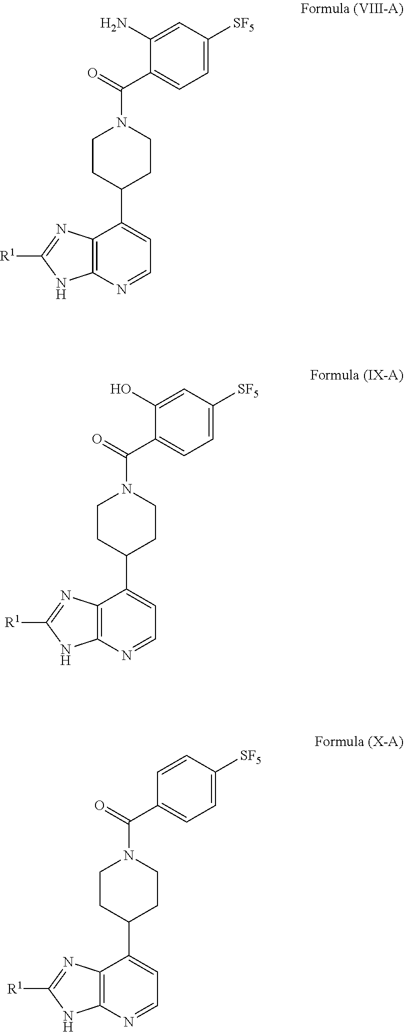

or a pharmaceutically acceptable salt thereof, wherein R¹ is as hereinbefore defined.

In embodiments, the compound is a compound of Formula (VIII-A), or a pharmaceutically acceptable salt thereof. In other embodiments, the compound is a compound of Formula (IX-A), or a pharmaceutically acceptable salt thereof. In other embodiments, the compound is a compound of Formula (X-A), or a pharmaceutically acceptable salt thereof.

In embodiments, the compound is as defined herein (e.g., a compound of Formula (I) or a pharmaceutically acceptable salt thereof), with the proviso that if R^{B} is —O(C₃-C₆)cycloalkyl or —NHBoc, then R¹ is not —(C₃-C₆)cycloalkyl or 4- to 6-membered monocyclic heterocycloalkyl. In embodiments, the compound is as defined herein (e.g., a compound of Formula (I) or a pharmaceutically acceptable salt thereof), with the proviso that if R^{B} is —O— cyclopropyl or —NHBoc, then R¹ is not cyclopropyl, oxetanyl, tetrahydrofuranyl, tetrahydropyranyl, or piperidinyl. In embodiments, the compound is as defined herein (e.g., a compound of Formula (I) or a pharmaceutically acceptable salt thereof), with the proviso that if R^{B} is —O-cyclopropyl, then R¹ is not

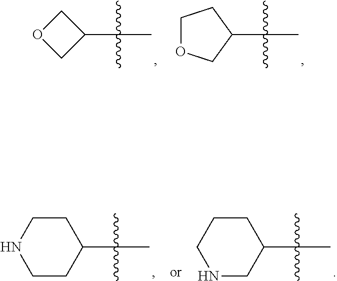 [SMILES: *C1COC1 ; *C1CCOC1 ; *C1CCNCC1 ; *C1CCCNC1]

In embodiments, the compound is as defined herein (e.g., a compound of Formula (I) or a pharmaceutically acceptable salt thereof), with the proviso that if R^{B} is —NHBoc, then R¹ is not

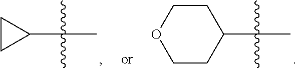

In embodiments, the compound (e.g., the compound of Formula (I)) is not as defined in WO 2022/187518 A1. In embodiments, the compound (e.g., the compound of Formula (I)) is not one or more of (e.g., any of) the compounds exemplified in WO 2022/187518 A1.

In embodiments, the compound (e.g., the compound of Formula (I)) is not one or more of (e.g., any of):
[2-amino-4-(trifluoromethoxy)phenyl]-[4-(2-tetrahydrofuran-3-yl-3H-imidazo[4,5-b]pyridin-7-yl)-1-piperidyl]methanone,
[2-amino-4-(trifluoromethoxy)phenyl]-[4-(2-tetrahydropyran-4-yl-3H-imidazo[4,5-b]pyridin-7-yl)-1-piperidyl]methanone,
5-[7-[1-[4-(trifluoromethoxy)benzoyl]-4-piperidyl]-3H-imidazo[4,5-b]pyridin-2-yl]piperidin-2-one,
[4-(3H-imidazo[4,5-b]pyridin-7-yl)-1-piperidyl]-[4-(trifluoromethoxy)phenyl]methanone,
[4-(2-morpholin-2-yl-3H-imidazo[4,5-b]pyridin-7-yl]-1-piperidyl]-[4-(trifluoromethoxy)phenyl]methanone,
[2-amino-4-(trifluoromethoxy)phenyl]-[4-(2-morpholin-2-yl-3H-imidazo[4,5-b]pyridin-7-yl)-1-piperidyl]methanone,
[4-[2-[(2R)-morpholin-2-yl]-3H-imidazo[4,5-b]pyridin-7-yl]-1-piperidyl]-[4-(trifluoromethoxy)phenyl]methanone;
[4-[2-(3-piperidyl)-3H-imidazo[4,5-b]pyridin-7-yl]-1-piperidyl]-[4-(trifluoromethoxy)phenyl]methanone;
5-[7-[1-[2-amino-4-(trifluoromethoxy)benzoyl]-4-piperidyl]-3H-imidazo[4,5-b]pyridin-2-yl]piperidin-2-one;
[2-amino-4-(trifluoromethoxy)phenyl]-[4-(6-fluoro-2-tetrahydropyran-4-yl-3H-imidazo[4,5-b]pyridin-7-yl)-1-piperidyl]methanone;
[2-amino-4-(trifluoromethoxy)phenyl]-[4-[2-(3-piperidyl)-3H-imidazo[4,5-b]pyridin-7-yl]-1-piperidyl]methanone;
(2-amino-4-trifluoromethoxyphenyl)[4-(3H-1,3,4-triazainden-7-yl)-1-piperidyl]methanone;
(1-phenyl-4-pyrazolyl)[4-(3H-1,3,4-triazainden-7-yl)-1-piperidyl]methanone;
(2-amino-4-trifluoromethoxyphenyl)[4-(2-cyclopropyl-imidazo[4,5-b]pyridin-7-yl)-1-piperidyl]methanone;
5-{7-[1-(2-amino-4-trifluoromethoxybenzoyl)-4-piperidyl]-imidazo[4,5-b]pyridin-2-yl}-2-piperidinone;
(2-amino-4-chlorophenyl){4-[2-(2-morpholinyl)-3H-1,3,4-triazainden-7-yl]-1-piperidyl}methanone;
5-{7-[1-(2-amino-4-chlorobenzoyl)-4-piperidyl]-3H-1,3,4-triazainden-2-yl}-2-piperidinone;
(2-amino-4-chlorophenyl){4-[2-(3-piperidyl)-3H-1,3,4-triazainden-7-yl]-1-piperidyl}methanone;
6-{7-[1-(4-trifluoromethoxy-benzoyl)-4-piperidyl]-imidazo[4,5-b]pyridin-2-yl}-3-morpholinone;
6-{7-[1-(2-amino-4-chlorobenzoyl)-4-piperidyl]-3H-1,3,4-triazainden-2-yl}-3-morpholinone;
3-({4-[2-(tetrahydro-2H-pyran-4-yl)-3H-1,3,4-triazainden-7-yl]-1-piperidyl}carbonyl)-6-(trifluoromethyl)-2(1H)-pyridinone;
(2-amino-4-trifluoromethoxyphenyl){4-[2-(1-methyl-3-pyrrolidinyl)-3H-1,3,4-triazainden-7-yl]-1-piperidyl}methanone;
6-{7-[1-(2-amino-4-trifluoromethoxy-benzoyl)-4-piperidyl]-imidazo[4,5-b]pyridin-2-yl}-3-morpholinone;
(2-amino-4-trifluoromethoxyphenyl){4-[6-fluoro-2-(1-methyl-4-pyrazolyl)-3H-1,3,4-triazainden-7-yl]-1-piperidyl}methanone;
(2-amino-4-chlorophenyl){4-[6-fluoro-2-(1-methyl-4-pyrazolyl)-3H-1,3,4-triazainden-7-yl]-1-piperidyl}methanone;
5-{7-[1-(2-amino-4-chlorobenzoyl)-4-piperidyl]-6-fluoro-3H-1,3,4-triazainden-2-yl}-2-piperidinone;
6-{7-[1-(2-amino-4-trifluoromethoxybenzoyl)-4-piperidyl]-6-fluoro-imidazo[4,5-b]pyridin-2-yl}-3-morpholinone;
3-{[4-(6-fluoro-3H-1,3,4-triazainden-7-yl)-1-piperidyl]carbonyl}-6-(trifluoromethyl)-2(1H)-pyridinone;
3-({4-[6-fluoro-2-(tetrahydro-2H-pyran-4-yl)-3H-1,3,4-triazainden-7-yl]-1-piperidyl}carbonyl)-6-(trifluoromethyl)-2(1H)-pyridinone;
4-{[4-(6-fluoro-3H-1,3,4-triazainden-7-yl)-1-piperidyl]carbonyl}-1-(2,2,2-trifluoroethyl)-2(1H)-pyridinone;
3-{[4-(6-fluoro-3H-1,3,4-triazainden-7-yl)-1-piperidyl]carbonyl}-1-methyl-6-(trifluoromethyl)-2(1H)-pyridinone;
{4-[6-fluoro-2-(1-methyl-4-pyrazolyl)-3H-1,3,4-triazainden-7-yl]-1-piperidyl}(p-trifluoromethoxyphenyl)methanone;
(2-amino-4-trifluoromethoxyphenyl){4-[6-fluoro-2-(3-piperidyl)-3H-1,3,4-triazainden-7-yl]-1-piperidyl}methanone;
(2-amino-4-chlorophenyl){4-[6-fluoro-2-(3-piperidyl)-3H-1,3,4-triazainden-7-yl]-1-piperidyl}methanone;
(2-amino-4-trifluoromethoxyphenyl){4-[6-fluoro-2-(2-morpholinyl)-3H-1,3,4-triazainden-7-yl]-1-piperidyl}methanone;
(2-amino-4-chlorophenyl){4-[6-fluoro-2-(2-morpholinyl)-3H-1,3,4-triazainden-7-yl]-1-piperidyl}methanone;
6-{7-[1-(2-amino-4-chlorobenzoyl)-4-piperidyl]-6-fluoro-3H-1,3,4-triazainden-2-yl}-3-morpholinone;
(2-amino-4-chloro-5-fluorophenyl){4-[6-fluoro-2-(1-methyl-4-pyrazolyl)-3H-1,3,4-triazainden-7-yl]-1-piperidyl}methanone;
4-{7-[1-(2-amino-4-trifluoromethoxy-benzoyl)-4-piperidyl]-6-fluoro-imidazo[4,5-b]pyridin-2-yl}-1λ6-1,1-thianedione;
{4-[6-fluoro-2-(tetrahydro-2H-pyran-4-yl)-3H-1,3,4-triazainden-7-yl]-1-piperidyl}(4-trifluoromethoxyphenyl)methanone;
(6-indolyl)[4-(3H-1,3,4-triazainden-7-yl)-1-piperidyl]methanone;
{4-[6-fluoro-2-(3-piperidyl)-3H-1,3,4-triazainden-7-yl]-1-piperidyl}(4-trifluoromethoxyphenyl)methanone;
1-(4-{7-[1-(2-amino-4-trifluoromethoxy-benzoyl)-4-piperidyl]-6-fluoro-imidazo[4,5-b]pyridin-2-yl}-1-piperidyl)-1-ethanone;
(2-amino-4-trifluoromethoxyphenyl){4-[6-fluoro-2-(4-piperidyl)-3H-1,3,4-triazainden-7-yl]-1-piperidyl}methanone;
(2-amino-4-chloro-5-fluorophenyl){4-[6-fluoro-2-(1-methyl-4-piperidyl)-3H-1,3,4-triazainden-7-yl]-1-piperidyl}methanone;
(2-amino-4-chloro-5-fluorophenyl){4-[6-fluoro-2-(4-piperidyl)-3H-1,3,4-triazainden-7-yl]-1-piperidyl}methanone;
(2-amino-4-chloro-5-fluorophenyl){4-[6-fluoro-2-(3-piperidyl)-3H-1,3,4-triazainden-7-yl]-1-piperidyl}methanone;
(2-amino-4-trifluoromethoxyphenyl){4-[6-fluoro-2-(3-fluoro-1-methyl-4-piperidyl)-3H-1,3,4-triazainden-7-yl]-1-piperidyl}methanone;
(2-amino-4-trifluoromethoxyphenyl){4-[6-fluoro-2-(1-methyl-4-piperidyl)-3H-1,3,4-triazainden-7-yl]-1-piperidyl}methanone;
{4-[6-fluoro-2-(3-oxetanyl)-3H-1,3,4-triazainden-7-yl]-1-piperidyl}(p-trifluoromethoxyphenyl)methanone;
{4-[6-fluoro-2-(2-morpholinyl)-3H-1,3,4-triazainden-7-yl]-1-piperidyl}(p-trifluoromethoxyphenyl)methanone;
{4-[6-fluoro-2-(4-piperidyl)-3H-1,3,4-triazainden-7-yl]-1-piperidyl}(p-trifluoromethoxyphenyl)methanone;
7-[1-(2-amino-4-chlorobenzoyl)-4-piperidyl]-1,3-dihydro-1,3,4-triaza-2-indenone;
7-[1-(4-trifluoromethoxy-benzoyl)-4-piperidyl]-imidazo[4,5-b]pyridin-2-one;
7-[1-(2-amino-4-trifluoromethoxy-benzoyl)-4-piperidyl]-imidazo[4,5-b]pyridin-2-one;
(6-indolyl)[4-(3H-1,3,4-triazainden-7-yl)-1-piperidyl]methanone;
(1-methyl-1H-indazol-5-yl)[4-(3H-1,3,4-triazainden-7-yl)-1-piperidyl]methanone;
(4-amino-3-methyl-5-isothiazolyl)[4-(3H-1,3,4-triazainden-7-yl)-1-piperidyl]methanone;
(5-bromo-2-pyrimidinyl)[4-(3H-1,3,4-triazainden-7-yl)-1-piperidyl]methanone;
(4-hydroxy-7-methyl-1,8-diaza-3-naphthyl)[4-(3H-1,3,4-triazainden-7-yl)-1-piperidyl]methanone;
[4-(3H-1,3,4-triazainden-7-yl)-1-piperidyl][5-(trifluoromethyl)-3-pyrazolyl]methanone;
(3-amino-4-bromo-5-fluorophenyl)[4-(3H-1,3,4-triazainden-7-yl)-1-piperidyl]methanone;
[1-(tert-butyl)-3-pyrrolyl][4-(3H-1,3,4-triazainden-7-yl)-1-piperidyl]methanone;
(1-methyl-4-imidazolyl)[4-(3H-1,3,4-triazainden-7-yl)-1-piperidyl]methanone;
7-[1-(2-naphthylsulfonyl)-4-piperidyl]-3H-1,3,4-triazaindene;
7-[1-(4-methoxyphenylsulfonyl)-4-piperidyl]-3H-1,3,4-triazaindene;
7-[1-(5-methyl-2-thienylsulfonyl)-4-piperidyl]-3H-1,3,4-triazaindene;
7-{1-[m-(trifluoromethyl)phenylsulfonyl]-4-piperidyl}-3H-1,3,4-triazaindene;
(6-methyl-2-naphthyl)[4-(3H-1,3,4-triazainden-7-yl)-1-piperidyl]methanone;
(3-amino-2-naphthyl){4-[2-(trifluoromethyl)-3H-1,3,4-triazainden-7-yl]-1-piperidyl}methanone;
(5-cyclopentyl-2-thienyl)[4-(3H-1,3,4-triazainden-7-yl)-1-piperidyl]methanone;
7-{1-[(3-quinolyl)carbonyl]-4-piperidyl}-1,3-dihydro-1,3,4-triaza-2-indenone;
7-{1-[(6-isoquinolyl)carbonyl]-4-piperidyl}-1,3-dihydro-1,3,4-triaza-2-indenone;
7-{1-[(2-quinolyl)carbonyl]-4-piperidyl}-1,3-dihydro-1,3,4-triaza-2-indenone;
7-{1-[(1,3-benzothiazol-6-yl)carbonyl]-4-piperidyl}-1,3-dihydro-1,3,4-triaza-2-indenone;
7-[1-(5-ethyl-2-thenoyl)-4-piperidyl]-1,3-dihydro-1,3,4-triaza-2-indenone;
7-[3-hydroxy-1-(5-isopropyl-3-thenoyl)-4-piperidyl]-1,3-dihydro-1,3,4-triaza-2-indenone;
7-[1-(2,4-difluoro-3-anisoyl)-4-piperidyl]-1,3-dihydro-1,3,4-triaza-2-indenone;
7-{1-[(2-methyl-1,3-thiazol-5-yl)carbonyl]-4-piperidyl}-1,3-dihydro-1,3,4-triaza-2-indenone;
7-{1-[(3-methoxy-5-isoxazolyl)carbonyl]-4-piperidyl}-1,3-dihydro-1,3,4-triaza-2-indenone;
7-[1-(2-hydroxy-4,6-dimethyl-nicotinoyl)-4-piperidyl]-1,3-dihydro-1,3,4-triaza-2-indenone;
6-fluoro-7-{1-[(2-methyl-1H-1,3-benzimidazol-6-yl)carbonyl]-4-piperidyl}-1,3-dihydro-1,3,4-triaza-2-indenone;
2-[4-(2-oxo-1,3-dihydro-1,3,4-triaza-7-indenyl)-1-piperidylsulfonyl]benzonitrile;
7-[1-(phenylsulfonyl)-4-piperidyl]-1,3-dihydro-1,3,4-triaza-2-indenone;
7-{1-[(4-oxo-3-1H-quinolyl)carbonyl]-4-piperidyl}-1,3-dihydro-1,3,4-triaza-2-indenone;
7-[1-(5-cyclopropyl-2-thenoyl)-4-piperidyl]-1,3-dihydro-1,3,4-triaza-2-indenone; and
7-(1-{[1-(4-pyridyl)-3-pyrazolyl]carbonyl}-4-piperidyl)-1,3-dihydro-1,3,4-triaza-2-indenone.

In embodiments, the compound (e.g., the compound of Formula (I)) is not one or more of (e.g., any of):
(2-amino-4-cyclopropoxyphenyl){4-[6-fluoro-2-(1-methyl-3-piperidyl)-3H-1,3,4-triazainden-7-yl]-1-piperidyl}methanone;
(2-amino-4-cyclopropoxyphenyl){4-[6-fluoro-2-(3-piperidyl)-3H-1,3,4-triazainden-7-yl]-1-piperidyl}methanone;
(4-cyclopropoxyphenyl){4-[6-fluoro-2-(4-piperidyl)-3H-1,3,4-triazainden-7-yl]-1-piperidyl}methanone;
(4-cyclopropoxyphenyl){4-[6-fluoro-2-(1-methyl-4-piperidyl)-3H-1,3,4-triazainden-7-yl]-1-piperidyl}methanone;
(2-amino-4-cyclopropoxyphenyl){4-[6-fluoro-2-(4-piperidyl)-3H-1,3,4-triazainden-7-yl]-1-piperidyl}methanone;
(4-cyclopropoxyphenyl){4-[6-fluoro-2-(tetrahydro-3-furyl)-3H-1,3,4-triazainden-7-yl]-1-piperidyl}methanone;
(4-cyclopropoxyphenyl){4-[6-fluoro-2-(3-oxetanyl)-3H-1,3,4-triazainden-7-yl]-1-piperidyl}methanone;
tert-butyl N-(2-[4-[2-(oxan-4-yl)-3H-imidazo[4,5-b]pyridin-7-yl]piperidine-1-carbonyl]-5-(trifluoromethoxy)phenyl) carbamate; and
tert-butyl N-[2-(4-[2-cyclopropyl-3H-imidazo[4,5-b]pyridin-7-yl]piperidine-1-carbonyl)-5-(trifluoromethoxy)phenyl]carbamate.

In embodiments, the compound is selected from the group consisting of
[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
[2-amino-4-(trifluoromethoxy)phenyl]-[4-(2-tetrahydrofuran-3-yl-3H-imidazo[4,5-b]pyridin-7-yl)-1-piperidyl]methanone,
[2-amino-4-(trifluoromethoxy)phenyl]-[4-(2-tetrahydropyran-4-yl-3H-imidazo[4,5-b]pyridin-7-yl)-1-piperidyl]methanone,
[4-[2-(4-piperidyl)-3H-imidazo[4,5-b]pyridin-7-yl]-1-piperidyl]-[4-(trifluoromethoxy)phenyl]methanone,
trans-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-aminocyclohexyl)-3H-imidazo[4,5-b]pyridin-7-yl]-1-piperidyl]-[4-(trifluoromethoxy)phenyl]methanone,
1-[3-[7-[1-[4-(trifluoromethoxy)benzoyl]-4-piperidyl]-3H-imidazo[4,5-b]pyridin-2-yl]azetidin-1-yl]ethanone,
[4-(2-tetrahydropyran-4-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(1-methyl-4-piperidyl)-3H-imidazo[4,5-b]pyridin-7-yl]-1-piperidyl]methanone,
[4-[2-(1-methyl-4-piperidyl)-3H-imidazo[4,5-b]pyridin-7-yl]-1-piperidyl]-[4-(trifluoromethoxy)phenyl]methanone,
[4-(2-methyl-3H-imidazo[4,5-b]pyridin-7-yl)-1-piperidyl]-[4-(trifluoromethoxy)phenyl]methanone,
[4-[2-(3-methoxy-1-bicyclo[1.1.1]pentanyl)-3H-imidazo[4,5-b]pyridin-7-yl]-1-piperidyl]-[4-(trifluoromethoxy)phenyl]methanone,
5-[7-[1-[4-(trifluoromethoxy)benzoyl]-4-piperidyl]-3H-imidazo[4,5-b]pyridin-2-yl]piperidin-2-one,
[4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone,
[4-(3H-imidazo[4,5-b]pyridin-7-yl)-1-piperidyl]-[4-(trifluoromethoxy)phenyl]methanone,
[4-[2-(oxetan-3-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone,
[4-[2-(azetidin-3-yl)-3H-imidazo[4,5-b]pyridin-7-yl]-1-piperidyl]-[4-(trifluoromethoxy)phenyl]methanone,
[4-(2-morpholin-2-yl-3H-imidazo[4,5-b]pyridin-7-yl]-1-piperidyl]-[4-(trifluoromethoxy)phenyl]methanone,
[4-[2-(1-methylpyrrolidin-3-yl)-3H-imidazo[4,5-b]pyridin-7-yl]-1-piperidyl]-[4-(trifluoromethoxy)phenyl]methanone,
[4-(2-tetrahydrofuran-3-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone,
[2-amino-4-(trifluoromethoxy)phenyl]-[4-(2-morpholin-2-yl-3H-imidazo[4,5-b]pyridin-7-yl)-1-piperidyl]methanone,
[4-[2-(2,2-dimethyltetrahydropyran-4-yl)-3H-imidazo[4,5-b]pyridin-7-yl]-1-piperidyl]-[4-(trifluoromethoxy)phenyl]methanone,
[4-[2-(6,6-dimethyltetrahydropyran-3-yl)-3H-imidazo[4,5-b]pyridin-7-yl]-1-piperidyl]-[4-(trifluoromethoxy)phenyl]methanone,
[4-[2-(1,4-dioxan-2-yl)-3H-imidazo[4,5-b]pyridin-7-yl]-1-piperidyl]-[4-(trifluoromethoxy)phenyl]methanone,
[4-(2-tetrahydrofuran-2-yl-3H-imidazo[4,5-b]pyridin-7-yl)-1-piperidyl]-[4-(trifluoromethoxy)phenyl]methanone,
[4-[2-(4-methylmorpholin-2-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(5,5-difluoro-3-piperidyl)-3H-imidazo[4,5-b]pyridin-7-yl]-1-piperidyl]methanone,
[2-amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone,
[4-[2-[1-(2,2-difluoroethyl)azetidin-3-yl]-3H-imidazo[4,5-b]pyridin-7-yl]-1-piperidyl]-[4-(trifluoromethoxy)phenyl]methanone,
[4-[2-(oxetan-3-ylmethyl)-3H-imidazo[4,5-b]pyridin-7-yl]-1-piperidyl]-[4-(trifluoromethoxy)phenyl]methanone,
(4-chloro-1H-indol-7-yl)-[4-(2-tetrahydropyran-4-yl-3H-imidazo[4,5-b]pyridin-7-yl)-1-piperidyl]methanone,
[2-nitro-4-(pentafluoro-λ⁶-sulfanyl)phenyl]-[4-(2-tetrahydropyran-4-yl-3H-imidazo[4,5-b]pyridin-7-yl)-1-piperidyl]methanone,
[4-[2-(tetrahydrofuran-3-ylmethyl)-3H-imidazo[4,5-b]pyridin-7-yl]-1-piperidyl]-[4-(trifluoromethoxy)phenyl]methanone,
[4-[2-(oxepan-4-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(oxepan-4-yl)-3H-imidazo[4,5-b]pyridin-7-yl]-1-piperidyl]methanone,
[4-[2-(2-oxaspiro[3.3]heptan-6-ylmethyl)-3H-imidazo[4,5-b]pyridin-7-yl]-1-piperidyl]-[4-(trifluoromethoxy)phenyl]methanone,
[4-(1,1,2,2,2-pentafluoroethyl)phenyl]-[4-(2-tetrahydropyran-4-yl-3H-imidazo[4,5-b]pyridin-7-yl)-1-piperidyl]methanone,
[4-(pentafluoro-λ⁶-sulfanyl)phenyl]-[4-(2-tetrahydropyran-4-yl-3H-imidazo[4,5-b]pyridin-7-yl)-1-piperidyl]methanone,
(cis)-[4-(2-tetrahydropyran-4-yl-3H-imidazo[4,5-b]pyridin-7-yl)-1-piperidyl]-[4-(2,2,2-trifluoroethyl)cyclohexyl]methanone,
(trans)-[4-(2-tetrahydropyran-4-yl-3H-imidazo[4,5-b]pyridin-7-yl)-1-piperidyl]-[4-(2,2,2-trifluoroethyl)cyclohexyl]methanone,
[2-amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone,
[2-amino-4-(trifluoromethoxy)phenyl]-[4-[2-(oxetan-3-ylmethyl)-3H-imidazo[4,5-b]pyridin-7-yl]-1-piperidyl]methanone,
(4-methoxy-1H-indol-7-yl)-[4-(2-tetrahydropyran-4-yl-3H-imidazo[4,5-b]pyridin-7-yl)-1-piperidyl]methanone,
6-[4-(2-tetrahydropyran-4-yl-3H-imidazo[4,5-b]pyridin-7-yl)piperidine-1-carbonyl]-3-(trifluoromethyl)-1H-pyridin-2-one,
[4-(2-tetrahydropyran-4-yl-3H-imidazo[4,5-b]pyridin-7-yl)-1-piperidyl]-[4-(2,2,2-trifluoroethyl)phenyl]methanone,
[4-(trifluoromethoxy)phenyl]-[4-[2-(trifluoromethyl)-3H-imidazo[4,5-b]pyridin-7-yl]-1-piperidyl]methanone,
[4-(2-tetrahydropyran-2-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-(2-tetrahydropyran-2-yl-3H-imidazo[4,5-b]pyridin-7-yl)-1-piperidyl]methanone,
(3-methoxy-1-bicyclo[1.1.1]pentanyl)-[4-(2-tetrahydropyran-4-yl-3H-imidazo[4,5-b]pyridin-7-yl)-1-piperidyl]methanone,
[4-(2-tetrahydropyran-4-yl-3H-imidazo[4,5-b]pyridin-7-yl)-1-piperidyl]-[3-(trifluoromethyl)-1-bicyclo[1.1.1]pentanyl]methanone,
[4-(2-tetrahydropyran-4-yl-3H-imidazo[4,5-b]pyridin-7-yl)-1-piperidyl]-[4-[1-(trifluoromethyl)cyclopropyl]phenyl]methanone,
[4-[2-(5,5-difluoro-3-piperidyl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(tetrahydropyran-4-ylmethyl)-3H-imidazo[4,5-b]pyridin-7-yl]-1-piperidyl]methanone,
[4-(2-pyrrolidin-3-yl-3H-imidazo[4,5-b]pyridin-7-yl)-1-piperidyl]-[4-(trifluoromethoxy)phenyl]methanone,
[4-[2-[(1-methyl-4-piperidyl)methyl]-3H-imidazo[4,5-b]pyridin-7-yl]-1-piperidyl]-[4-(trifluoromethoxy)phenyl]methanone,
[2-amino-4-(trifluoromethoxy)phenyl]-[4-(2-tetrahydropyran-3-yl-3H-imidazo[4,5-b]pyridin-7-yl)-1-piperidyl]methanone,
[4-[2-(tetrahydropyran-4-ylmethyl)-3H-imidazo[4,5-b]pyridin-7-yl]-1-piperidyl]-[4-(trifluoromethoxy)phenyl]methanone,
[4-(2-tetrahydropyran-3-yl-3H-imidazo[4,5-b]pyridin-7-yl)-1-piperidyl]-[4-(trifluoromethoxy)phenyl]methanone, and
[4-[2-[1-(2,2-difluoroethyl)-4-piperidyl]-3H-imidazo[4,5-b]pyridin-7-yl]-1-piperidyl]-[4-(trifluoromethoxy)phenyl]methanone,
   and the pharmaceutically acceptable salts thereof.

In embodiments, the compound is selected from the group consisting of:
[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
[4-[2-(4-piperidyl)-3H-imidazo[4,5-b]pyridin-7-yl]-1-piperidyl]-[4-(trifluoromethoxy)phenyl]methanone,
trans-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-aminocyclohexyl)-3H-imidazo[4,5-b]pyridin-7-yl]-1-piperidyl]-[4-(trifluoromethoxy)phenyl]methanone,
1-[3-[7-[1-[4-(trifluoromethoxy)benzoyl]-4-piperidyl]-3H-imidazo[4,5-b]pyridin-2-yl]azetidin-1-yl]ethanone,
[4-(2-tetrahydropyran-4-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(1-methyl-4-piperidyl)-3H-imidazo[4,5-b]pyridin-7-yl]-1-piperidyl]methanone,
[4-[2-(1-methyl-4-piperidyl)-3H-imidazo[4,5-b]pyridin-7-yl]-1-piperidyl]-[4-(trifluoromethoxy)phenyl]methanone,
[4-(2-methyl-3H-imidazo[4,5-b]pyridin-7-yl)-1-piperidyl]-[4-(trifluoromethoxy)phenyl]methanone,
[4-[2-(3-methoxy-1-bicyclo[1.1.1]pentanyl)-3H-imidazo[4,5-b]pyridin-7-yl]-1-piperidyl]-[4-(trifluoromethoxy)phenyl]methanone,
[4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone,
[4-[2-(oxetan-3-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone,
[4-[2-(azetidin-3-yl)-3H-imidazo[4,5-b]pyridin-7-yl]-1-piperidyl]-[4-(trifluoromethoxy)phenyl]methanone,
[4-[2-(1-methylpyrrolidin-3-yl)-3H-imidazo[4,5-b]pyridin-7-yl]-1-piperidyl]-[4-(trifluoromethoxy)phenyl]methanone,
[4-(2-tetrahydrofuran-3-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone,
[4-[2-(2,2-dimethyltetrahydropyran-4-yl)-3H-imidazo[4,5-b]pyridin-7-yl]-1-piperidyl]-[4-(trifluoromethoxy)phenyl]methanone,
[4-[2-(6,6-dimethyltetrahydropyran-3-yl)-3H-imidazo[4,5-b]pyridin-7-yl]-1-piperidyl]-[4-(trifluoromethoxy)phenyl]methanone,
[4-[2-(1,4-dioxan-2-yl)-3H-imidazo[4,5-b]pyridin-7-yl]-1-piperidyl]-[4-(trifluoromethoxy)phenyl]methanone,
[4-(2-tetrahydrofuran-2-yl-3H-imidazo[4,5-b]pyridin-7-yl)-1-piperidyl]-[4-(trifluoromethoxy)phenyl]methanone,
[4-[2-(4-methylmorpholin-2-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(5,5-difluoro-3-piperidyl)-3H-imidazo[4,5-b]pyridin-7-yl]-1-piperidyl]methanone,
[2-amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone,
[4-[2-[1-(2,2-difluoroethyl)azetidin-3-yl]-3H-imidazo[4,5-b]pyridin-7-yl]-1-piperidyl]-[4-(trifluoromethoxy)phenyl]methanone,
[4-[2-(oxetan-3-ylmethyl)-3H-imidazo[4,5-b]pyridin-7-yl]-1-piperidyl]-[4-(trifluoromethoxy)phenyl]methanone,
(4-chloro-1H-indol-7-yl)-[4-(2-tetrahydropyran-4-yl-3H-imidazo[4,5-b]pyridin-7-yl)-1-piperidyl]methanone,
[2-nitro-4-(pentafluoro-λ⁶-sulfanyl)phenyl]-[4-(2-tetrahydropyran-4-yl-3H-imidazo[4,5-b]pyridin-7-yl)-1-piperidyl]methanone,
[4-[2-(tetrahydrofuran-3-ylmethyl)-3H-imidazo[4,5-b]pyridin-7-yl]-1-piperidyl]-[4-(trifluoromethoxy)phenyl]methanone,
[4-[2-(oxepan-4-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(oxepan-4-yl)-3H-imidazo[4,5-b]pyridin-7-yl]-1-piperidyl]methanone,
[4-[2-(2-oxaspiro[3.3]heptan-6-ylmethyl)-3H-imidazo[4,5-b]pyridin-7-yl]-1-piperidyl]-[4-(trifluoromethoxy)phenyl]methanone,
[4-(1,1,2,2,2-pentafluoroethyl)phenyl]-[4-(2-tetrahydropyran-4-yl-3H-imidazo[4,5-b]pyridin-7-yl)-1-piperidyl]methanone,
[4-(pentafluoro-λ⁶-sulfanyl)phenyl]-[4-(2-tetrahydropyran-4-yl-3H-imidazo[4,5-b]pyridin-7-yl)-1-piperidyl]methanone,
(cis)-[4-(2-tetrahydropyran-4-yl-3H-imidazo[4,5-b]pyridin-7-yl)-1-piperidyl]-[4-(2,2,2-trifluoroethyl)cyclohexyl]methanone,
(trans)-[4-(2-tetrahydropyran-4-yl-3H-imidazo[4,5-b]pyridin-7-yl)-1-piperidyl]-[4-(2,2,2-trifluoroethyl)cyclohexyl]methanone,
[2-amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone,
[2-amino-4-(trifluoromethoxy)phenyl]-[4-[2-(oxetan-3-ylmethyl)-3H-imidazo[4,5-b]pyridin-7-yl]-1-piperidyl]methanone,
(4-methoxy-1H-indol-7-yl)-[4-(2-tetrahydropyran-4-yl-3H-imidazo[4,5-b]pyridin-7-yl)-1-piperidyl]methanone,
6-[4-(2-tetrahydropyran-4-yl-3H-imidazo[4,5-b]pyridin-7-yl)piperidine-1-carbonyl]-3-(trifluoromethyl)-1H-pyridin-2-one,
[4-(2-tetrahydropyran-4-yl-3H-imidazo[4,5-b]pyridin-7-yl)-1-piperidyl]-[4-(2,2,2-trifluoroethyl)phenyl]methanone,
[4-(trifluoromethoxy)phenyl]-[4-[2-(trifluoromethyl)-3H-imidazo[4,5-b]pyridin-7-yl]-1-piperidyl]methanone,
[4-(2-tetrahydropyran-2-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-(2-tetrahydropyran-2-yl-3H-imidazo[4,5-b]pyridin-7-yl)-1-piperidyl]methanone,
(3-methoxy-1-bicyclo[1.1.1]pentanyl)-[4-(2-tetrahydropyran-4-yl-3H-imidazo[4,5-b]pyridin-7-yl)-1-piperidyl]methanone,
[4-(2-tetrahydropyran-4-yl-3H-imidazo[4,5-b]pyridin-7-yl)-1-piperidyl]-[3-(trifluoromethyl)-1-bicyclo[1.1.1]pentanyl]methanone,
[4-(2-tetrahydropyran-4-yl-3H-imidazo[4,5-b]pyridin-7-yl)-1-piperidyl]-[4-[1-(trifluoromethyl)cyclopropyl]phenyl]methanone,
[4-[2-(5,5-difluoro-3-piperidyl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(tetrahydropyran-4-ylmethyl)-3H-imidazo[4,5-b]pyridin-7-yl]-1-piperidyl]methanone,
[4-(2-pyrrolidin-3-yl-3H-imidazo[4,5-b]pyridin-7-yl)-1-piperidyl]-[4-(trifluoromethoxy)phenyl]methanone,
[4-[2-[(1-methyl-4-piperidyl)methyl]-3H-imidazo[4,5-b]pyridin-7-yl]-1-piperidyl]-[4-(trifluoromethoxy)phenyl]methanone,
[2-amino-4-(trifluoromethoxy)phenyl]-[4-(2-tetrahydropyran-3-yl-3H-imidazo[4,5-b]pyridin-7-yl)-1-piperidyl]methanone,
[4-[2-(tetrahydropyran-4-ylmethyl)-3H-imidazo[4,5-b]pyridin-7-yl]-1-piperidyl]-[4-(trifluoromethoxy)phenyl]methanone,
[4-(2-tetrahydropyran-3-yl-3H-imidazo[4,5-b]pyridin-7-yl)-1-piperidyl]-[4-(trifluoromethoxy)phenyl]methanone, and
[4-[2-[1-(2,2-difluoroethyl)-4-piperidyl]-3H-imidazo[4,5-b]pyridin-7-yl]-1-piperidyl]-[4-(trifluoromethoxy)phenyl]methanone,
   and the pharmaceutically acceptable salts thereof.

In embodiments, the compound is selected from the group consisting of:
[2-amino-4-(trifluoromethoxy)phenyl]-[4-[2-(oxetan-3-yl)-3H-imidazo[4,5-b]pyridin-7-yl]-1-piperidyl]methanone;
[4-[2-[tetrahydrofuran-2-yl]-3H-imidazo[4,5-b]pyridin-7-yl]-1-piperidyl]-[4-(trifluoromethoxy)phenyl]methanone;
[4-[2-(3-hydroxy-1-bicyclo[1.1.1]pentanyl)-3H-imidazo[4,5-b]pyridin-7-yl]-1-piperidyl]-[4-(trifluoromethoxy)phenyl]methanone;
[4-[2-[pyrrolidin-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[tetrahydropyran-2-yl]-3H-imidazo[4,5-b]pyridin-7-yl]-1-piperidyl]methanone;
2-naphthyl-[4-(2-tetrahydropyran-4-yl-3H-imidazo[4,5-b]pyridin-7-yl)-1-piperidyl]methanone;
1H-indazol-3-yl-[4-(2-tetrahydropyran-4-yl-3H-imidazo[4,5-b]pyridin-7-yl)-1-piperidyl]methanone;
[4-(2-tetrahydropyran-4-yl-3H-imidazo[4,5-b]pyridin-7-yl)-1-piperidyl]-[7-(trifluoromethyl)-4,5,6,7-tetrahydropyrazolo[1,5-a]pyrimidin-3-yl]methanone;
(1-methyl-5-phenyl-pyrazol-3-yl)-[4-(2-tetrahydropyran-4-yl-3H-imidazo[4,5-b]pyridin-7-yl)-1-piperidyl]methanone;
(3-amino-2-naphthyl)-[4-(2-tetrahydropyran-4-yl-3H-imidazo[4,5-b]pyridin-7-yl)-1-piperidyl]methanone;
(2-amino-3,4,5,6-tetrafluoro-phenyl)-[4-(2-tetrahydropyran-4-yl-3H-imidazo[4,5-b]pyridin-7-yl)-1-piperidyl]methanone;
[1-isopropyl-2-(trifluoromethyl)benzimidazol-5-yl]-[4-(2-tetrahydropyran-4-yl-3H-imidazo[4,5-b]pyridin-7-yl)-1-piperidyl]methanone;
(2-amino-4-methylsulfonyl-phenyl)-[4-(2-tetrahydropyran-4-yl-3H-imidazo[4,5-b]pyridin-7-yl)-1-piperidyl]methanone;
[3-(1,1-dioxo-1,2-thiazolidin-2-yl)phenyl]-[4-(2-tetrahydropyran-4-yl-3H-imidazo[4,5-b]pyridin-7-yl)-1-piperidyl]methanone;
[2-(3-hydroxyphenyl)cyclopropyl]-[4-(2-tetrahydropyran-4-yl-3H-imidazo[4,5-b]pyridin-7-yl)-1-piperidyl]methanone;
2-[2-chloro-4-(trifluoromethyl)phenoxy]-1-[4-(2-tetrahydropyran-4-yl-3H-imidazo[4,5-b]pyridin-7-yl)-1-piperidyl]propan-1-one;
(3-phenylcyclopentyl)-[4-(2-tetrahydropyran-4-yl-3H-imidazo[4,5-b]pyridin-7-yl)-1-piperidyl]methanone;
[4-hydroxy-1-[2-(trifluoromethyl)phenyl]pyrazol-3-yl]-[4-(2-tetrahydropyran-4-yl-3H-imidazo[4,5-b]pyridin-7-yl)-1-piperidyl]methanone;
[4-[2-[5,5-difluoro-3-piperidyl]-3H-imidazo[4,5-b]pyridin-7-yl]-1-piperidyl]-[4-(trifluoromethoxy)phenyl]methanone;
[3-amino-4-phenyl-5-(trifluoromethyl)-2-thienyl]-[4-(2-tetrahydropyran-4-yl-3H-imidazo[4,5-b]pyridin-7-yl)-1-piperidyl]methanone;
(7-hydroxy-1H-indol-2-yl)-[4-(2-tetrahydropyran-4-yl-3H-imidazo[4,5-b]pyridin-7-yl)-1-piperidyl]methanone;
[4-[2-(1-cyclopropyl-4-piperidyl)-3H-imidazo[4,5-b]pyridin-7-yl]-1-piperidyl]-[4-(trifluoromethoxy)phenyl]methanone;
1-[4-[7-[1-[4-(trifluoromethoxy)benzoyl]-4-piperidyl]-3H-imidazo[4,5-b]pyridin-2-yl]-1-piperidyl]ethanone;
[2-amino-4-(pentafluoro-λ⁶-sulfanyl)phenyl]-[4-(2-tetrahydropyran-4-yl-3H-imidazo[4,5-b]pyridin-7-yl)-1-piperidyl]methanone;
(cis)-[4-(2-tetrahydropyran-4-yl-3H-imidazo[4,5-b]pyridin-7-yl)-1-piperidyl]-[4-(trifluoromethoxy)cyclohexyl]methanone;
1-[4-[7-[1-[2-amino-4-(trifluoromethoxy)benzoyl]-4-piperidyl]-3H-imidazo[4,5-b]pyridin-2-yl]-1-piperidyl]ethanone;
[2-amino-4-(trifluoromethoxy)phenyl]-[4-[2-(1-cyclopropyl-4-piperidyl)-3H-imidazo[4,5-b]pyridin-7-yl]-1-piperidyl]methanone;
[4-(2-cyclohexyl-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-(1,4-dioxan-2-yl)-3H-imidazo[4,5-b]pyridin-7-yl]-1-piperidyl]methanone;
2-[4-(2-tetrahydropyran-4-yl-3H-imidazo[4,5-b]pyridin-7-yl)piperidine-1-carbonyl]benzothiophene-5-carbonitrile;
(5-amino-1-phenyl-pyrazol-4-yl)-[4-(2-tetrahydropyran-4-yl-3H-imidazo[4,5-b]pyridin-7-yl)-1-piperidyl]methanone;
2-[2-oxo-2-[4-(2-tetrahydropyran-4-yl-3H-imidazo[4,5-b]pyridin-7-yl)-1-piperidyl]ethyl]-4H-1,4-benzoxazin-3-one;
1-(3,4-difluorophenyl)-4-[4-(2-tetrahydropyran-4-yl-3H-imidazo[4,5-b]pyridin-7-yl)piperidine-1-carbonyl]pyrrolidin-2-one;
[4-(3-chloro-4-fluoro-phenyl)phenyl]-[4-(2-tetrahydropyran-4-yl-3H-imidazo[4,5-b]pyridin-7-yl)-1-piperidyl]methanone;
(7-amino-2-methyl-pyrazolo[1,5-a]pyrimidin-6-yl)-[4-(2-tetrahydropyran-4-yl-3H-imidazo[4,5-b]pyridin-7-yl)-1-piperidyl]methanone;
1-[(2-chlorophenyl)methyl]-4-[4-(2-tetrahydropyran-4-yl-3H-imidazo[4,5-b]pyridin-7-yl)piperidine-1-carbonyl]pyrrolidin-2-one;
1-(4-fluorophenyl)-4-[4-(2-tetrahydropyran-4-yl-3H-imidazo[4,5-b]pyridin-7-yl)piperidine-1-carbonyl]pyrrolidin-2-one;
[1-[(2-chlorophenyl)methyl]pyrazol-4-yl]-[4-(2-tetrahydropyran-4-yl-3H-imidazo[4,5-b]pyridin-7-yl)-1-piperidyl]methanone;
[4-(2-aminophenyl)phenyl]-[4-(2-tetrahydropyran-4-yl-3H-imidazo[4,5-b]pyridin-7-yl)-1-piperidyl]methanone;
(3,6-dichloroimidazo[1,2-a]pyridin-2-yl)-[4-(2-tetrahydropyran-4-yl-3H-imidazo[4,5-b]pyridin-7-yl)-1-piperidyl]methanone;
(4-amino-1-ethyl-pyrazol-3-yl)-[4-(2-tetrahydropyran-4-yl-3H-imidazo[4,5-b]pyridin-7-yl)-1-piperidyl]methanone;
(3-aminoquinoxalin-2-yl)-[4-(2-tetrahydropyran-4-yl-3H-imidazo[4,5-b]pyridin-7-yl)-1-piperidyl]methanone;
2-[4-(2-tetrahydropyran-4-yl-3H-imidazo[4,5-b]pyridin-7-yl)piperidine-1-carbonyl]spiro[cyclopropane-1,3′-indoline]-2′-one;
[1-(2-chlorophenyl)pyrrolidin-3-yl]-[4-(2-tetrahydropyran-4-yl-3H-imidazo[4,5-b]pyridin-7-yl)-1-piperidyl]methanone;
(2-amino-4,5-dichloro-phenyl)-[4-(2-tetrahydropyran-4-yl-3H-imidazo[4,5-b]pyridin-7-yl)-1-piperidyl]methanone;
[1-(4-fluorophenyl)imidazol-4-yl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-(1-methoxy-1-methyl-ethyl)-3H-imidazo[4,5-b]pyridin-7-yl]-1-piperidyl]methanone;
[2-amino-4-(trifluoromethoxy)phenyl]-[4-[2-(1-hydroxy-1-methyl-ethyl)-3H-imidazo[4,5-b]pyridin-7-yl]-1-piperidyl]methanone;
[2-amino-4-(trifluoromethoxy)phenyl]-[4-(2-cyclohexyl-3H-imidazo[4,5-b]pyridin-7-yl)-1-piperidyl]methanone;
[2-amino-4-(trifluoromethoxy)phenyl]-[4-[2-(3-methoxypropyl)-3H-imidazo[4,5-b]pyridin-7-yl]-1-piperidyl]methanone;
[4-[2-(3-amino-1-bicyclo[1.1.1]pentanyl)-3H-imidazo[4,5-b]pyridin-7-yl]-1-piperidyl]-[4-(trifluoromethoxy)phenyl]methanone;
[4-[2-(3-amino-1-bicyclo[1.1.1]pentanyl)-3H-imidazo[4,5-b]pyridin-7-yl]-1-piperidyl]-[2-amino-4-(trifluoromethoxy)phenyl]methanone;
(trans)-[4-(2-tetrahydropyran-4-yl-3H-imidazo[4,5-b]pyridin-7-yl)-1-piperidyl]-[4-(trifluoromethoxy)cyclohexyl]methanone;
[3-fluoro-4-(trifluoromethoxy)phenyl]-[4-(2-tetrahydropyran-4-yl-3H-imidazo[4,5-b]pyridin-7-yl)-1-piperidyl]methanone;
(cis)-[2-amino-4-(trifluoromethoxy)phenyl]-[4-[2-(3-methoxycyclobutyl)-3H-imidazo[4,5-b]pyridin-7-yl]-1-piperidyl]methanone;
(trans)-[2-amino-4-(trifluoromethoxy)phenyl]-[4-[2-(3-methoxycyclobutyl)-3H-imidazo[4,5-b]pyridin-7-yl]-1-piperidyl]methanone;
[4-[2-[(4-methylpiperazin-1-yl)methyl]-3H-imidazo[4,5-b]pyridin-7-yl]-1-piperidyl]-[4-(trifluoromethoxy)phenyl]methanone;
[4-[2-[2-(5-chloro-2-hydroxy-phenyl)thiazol-4-yl]-3H-imidazo[4,5-b]pyridin-7-yl]-1-piperidyl]-[4-(trifluoromethoxy)phenyl]methanone;
[4-[2-(2-fluoro-5-hydroxy-phenyl)-3H-imidazo[4,5-b]pyridin-7-yl]-1-piperidyl]-[4-(trifluoromethoxy)phenyl]methanone;
N-[3-[7-[1-[4-(trifluoromethoxy)benzoyl]-4-piperidyl]-3H-imidazo[4,5-b]pyridin-2-yl]phenyl]tetrahydrofuran-2-carboxamide;
[4-[2-[5-(hydroxymethyl)isoxazol-3-yl]-3H-imidazo[4,5-b]pyridin-7-yl]-1-piperidyl]-[4-(trifluoromethoxy)phenyl]methanone;
1-ethyl-3-hydroxy-6-[7-[1-[4-(trifluoromethoxy)benzoyl]-4-piperidyl]-3H-imidazo[4,5-b]pyridin-2-yl]quinoxalin-2-one;
[3-(2-tetrahydropyran-4-yl-3H-imidazo[4,5-b]pyridin-7-yl)pyrrolidin-1-yl]-[4-(trifluoromethoxy)phenyl]methanone;
[2-amino-4-(trifluoromethoxy)phenyl]-[3-(2-tetrahydropyran-4-yl-3H-imidazo[4,5-b]pyridin-7-yl)pyrrolidin-1-yl]methanone;
(4-bromophenyl)-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[1,4-dioxan-2-yl]-3H-imidazo[4,5-b]pyridin-7-yl]-1-piperidyl]methanone;
4-[7-[1-[2-amino-4-(trifluoromethoxy)benzoyl]-4-piperidyl]-3H-imidazo[4,5-b]pyridin-2-yl]cyclohexanone;
[4-(2-tetrahydropyran-4-yl-3H-imidazo[4,5-b]pyridin-7-yl)-1-piperidyl]-(4-trimethylsilylphenyl)methanone;
(cis)-[4-[2-(3-hydroxycyclobutyl)-3H-imidazo[4,5-b]pyridin-7-yl]-1-piperidyl]-[4-(trifluoromethoxy)phenyl]methanone;
(trans)-[4-[2-(3-hydroxycyclobutyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-(3-methoxy-1-bicyclo[1.1.1]pentanyl)-3H-imidazo[4,5-b]pyridin-7-yl]-1-piperidyl]methanone;
(trans)-[4-(trifluoromethoxy)phenyl]-[4-[2-(4-methoxycyclohexyl)-3H-imidazo[4,5-b]pyridin-7-yl]-1-piperidyl]methanone;
(cis)-[4-(trifluoromethoxy)phenyl]-[4-[2-(4-methoxycyclohexyl)-3H-imidazo[4,5-b]pyridin-7-yl]-1-piperidyl]methanone;
(trans)-[2-amino-4-(trifluoromethoxy)phenyl]-[4-[2-(4-methoxycyclohexyl)-3H-imidazo[4,5-b]pyridin-7-yl]-1-piperidyl]methanone;
(cis)-[2-amino-4-(trifluoromethoxy)phenyl]-[4-[2-(4-methoxycyclohexyl)-3H-imidazo[4,5-b]pyridin-7-yl]-1-piperidyl]methanone;
(2,2-difluoro-1,3-benzodioxol-5-yl)-[4-(2-tetrahydropyran-4-yl-3H-imidazo[4,5-b]pyridin-7-yl)-1-piperidyl]methanone;
(4-bromo-1H-indol-7-yl)-[4-(2-tetrahydropyran-4-yl-3H-imidazo[4,5-b]pyridin-7-yl)-1-piperidyl]methanone;
[4-(2-tetrahydropyran-4-yl-3H-imidazo[4,5-b]pyridin-7-yl)-1-piperidyl]-[2-(trifluoromethyl)-1H-indol-6-yl]methanone;
[4-(cyclopropoxy)phenyl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[5,5-difluoro-3-piperidyl]-3H-imidazo[4,5-b]pyridin-7-yl]-1-piperidyl]methanone;
[2-amino-4-(trifluoromethoxy)phenyl]-[3-(2-tetrahydropyran-4-yl-3H-imidazo[4,5-b]pyridin-7-yl)pyrrolidin-1-yl]methanone;
2-tetrahydropyran-4-yl-7-[1-[4-(trifluoromethoxy)phenyl]sulfonyl-4-piperidyl]-3H-imidazo[4,5-b]pyridine;
[2-amino-4-(trifluoromethoxy)phenyl]-[4-[2-[oxepan-4-yl]-3H-imidazo[4,5-b]pyridin-7-yl]-1-piperidyl]methanone;
[4-[2-[rac-(2R,3S)-3-aminotetrahydrofuran-2-yl]-3H-imidazo[4,5-b]pyridin-7-yl]-1-piperidyl]-[4-(trifluoromethoxy)phenyl]methanone;
(cis)-[2-amino-4-(trifluoromethoxy)phenyl]-[4-[2-(3-hydroxycyclobutyl)-3H-imidazo[4,5-b]pyridin-7-yl]-1-piperidyl]methanone;
(trans)-[2-amino-4-(trifluoromethoxy)phenyl]-[4-[2-(3-hydroxycyclobutyl)-3H-imidazo[4,5-b]pyridin-7-yl]-1-piperidyl]methanone;
[3-(2-tetrahydropyran-4-yl-3H-imidazo[4,5-b]pyridin-7-yl)pyrrolidin-1-yl]-[4-(trifluoromethoxy)phenyl]methanone;
[2-amino-4-(trifluoromethoxy)phenyl]-[4-(2-morpholino-3H-imidazo[4,5-b]pyridin-7-yl)-1-piperidyl]methanone;
[2-amino-4-(trifluoromethoxy)phenyl]-[4-[2-(2-methylmorpholin-2-yl)-3H-imidazo[4,5-b]pyridin-7-yl]-1-piperidyl]methanone;
[4-[2-[6,6-dimethylmorpholin-2-yl]-3H-imidazo[4,5-b]pyridin-7-yl]-1-piperidyl]-[4-(trifluoromethoxy)phenyl]methanone;
[4-[2-[6,6-dimethylmorpholin-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[tetrahydrofuran-2-yl]-3H-imidazo[4,5-b]pyridin-7-yl]-1-piperidyl]methanone;
[4-[2-(7-oxa-4-azaspiro[2.5]octan-6-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[6,6-dimethylmorpholin-2-yl]-3H-imidazo[4,5-b]pyridin-7-yl]-1-piperidyl]methanone;
[2-amino-4-(trifluoromethoxy)phenyl]-[4-[2-[6,6-dimethylmorpholin-2-yl]-3H-imidazo[4,5-b]pyridin-7-yl]-1-piperidyl]methanone;
[4-[2-[1-(oxetan-3-yl)-4-piperidyl]-3H-imidazo[4,5-b]pyridin-7-yl]-1-piperidyl]-[4-(trifluoromethoxy)phenyl]methanone;
[4-[2-(2-oxabicyclo[2.2.2]octan-4-yl)-3H-imidazo[4,5-b]pyridin-7-yl]-1-piperidyl]-[4-(trifluoromethoxy)phenyl]methanone;
[4-(2-morpholino-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-[tetrahydrofuran-3-yl]-3H-imidazo[4,5-b]pyridin-7-yl]-1-piperidyl]methanone;
[2-amino-4-(trifluoromethoxy)phenyl]-[4-[2-[1-(oxetan-3-yl)-4-piperidyl]-3H-imidazo[4,5-b]pyridin-7-yl]-1-piperidyl]methanone;
[2-amino-3-fluoro-4-(trifluoromethoxy)phenyl]-[4-(2-tetrahydropyran-4-yl-3H-imidazo[4,5-b]pyridin-7-yl)-1-piperidyl]methanone;
[4-[2-[rac-(2R,3S)-3-aminotetrahydrofuran-2-yl]-3H-imidazo[4,5-b]pyridin-7-yl]-1-piperidyl]-[2-amino-4-(trifluoromethoxy)phenyl]methanone;
(cis)-[4-(2-tetrahydropyran-4-yl-3H-imidazo[4,5-b]pyridin-7-yl)cyclohexyl]-[4-(trifluoromethoxy)-1-piperidyl]methanone;
(trans)-[4-(2-tetrahydropyran-4-yl-3H-imidazo[4,5-b]pyridin-7-yl)cyclohexyl]-[4-(trifluoromethoxy)-1-piperidyl]methanone;
[4-[2-(4-amino-1-methyl-2-oxabicyclo[2.1.1]hexan-3-yl)-3H-imidazo[4,5-b]pyridin-7-yl]-1-piperidyl]-[2-amino-4-(trifluoromethoxy)phenyl]methanone;
(trans)-[4-[2-(3-methoxycyclobutyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-(7-oxa-4-azaspiro[2.5]octan-6-yl)-3H-imidazo[4,5-b]pyridin-7-yl]-1-piperidyl]methanone;
[2-amino-4-(trifluoromethoxy)phenyl]-[4-[2-(2-oxabicyclo[2.2.2]octan-4-yl)-3H-imidazo[4,5-b]pyridin-7-yl]-1-piperidyl]methanone;
[4-(2-cyclopent-3-en-1-yl-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone;
(2-amino-4-bromo-phenyl)-[4-(2-tetrahydropyran-4-yl-3H-imidazo[4,5-b]pyridin-7-yl)-1-piperidyl]methanone;
[4-[2-(4-amino-1-methyl-2-oxabicyclo[2.1.1]hexan-3-yl)-3H-imidazo[4,5-b]pyridin-7-yl]-1-piperidyl]-[4-(trifluoromethoxy)phenyl]methanone;
N-[2-[4-(2-tetrahydropyran-4-yl-3H-imidazo[4,5-b]pyridin-7-yl)piperidine-1-carbonyl]-5-(trifluoromethoxy)phenyl]acetamide;
(trans)-[4-[2-(3-methoxycyclobutyl)-3H-imidazo[4,5-b]pyridin-7-yl]-1-piperidyl]-[4-(trifluoromethoxy)phenyl]methanone;
[4-[2-(1,2,3,6-tetrahydropyridin-5-yl)-3H-imidazo[4,5-b]pyridin-7-yl]-1-piperidyl]-[4-(trifluoromethoxy)phenyl]methanone;
[4-[2-[4-phenylpyrrolidin-3-yl]-3H-imidazo[4,5-b]pyridin-7-yl]-1-piperidyl]-[4-(trifluoromethoxy)phenyl]methanone;
[4-[2-[4-fluoropyrrolidin-2-yl]-3H-imidazo[4,5-b]pyridin-7-yl]-1-piperidyl]-[4-(trifluoromethoxy)phenyl]methanone;
[4-[2-(2-amino-3,3,3-trifluoro-propyl)-3H-imidazo[4,5-b]pyridin-7-yl]-1-piperidyl]-[4-(trifluoromethoxy)phenyl]methanone;
[4-[2-[6-(aminomethyl)-3-pyridyl]-3H-imidazo[4,5-b]pyridin-7-yl]-1-piperidyl]-[4-(trifluoromethoxy)phenyl]methanone;
[4-[2-[rac-(3R,4S)-4-(4-pyridyl)pyrrolidin-3-yl]-3H-imidazo[4,5-b]pyridin-7-yl]-1-piperidyl]-[4-(trifluoromethoxy)phenyl]methanone;
[4-[2-(3-amino-2-bicyclo[2.2.1]hept-5-enyl)-3H-imidazo[4,5-b]pyridin-7-yl]-1-piperidyl]-[4-(trifluoromethoxy)phenyl]methanone;
[4-[2-[pyrrolidin-2-yl]-3H-imidazo[4,5-b]pyridin-7-yl]-1-piperidyl]-[4-(trifluoromethoxy)phenyl]methanone;
[4-[2-(3-amino-2-thienyl)-3H-imidazo[4,5-b]pyridin-7-yl]-1-piperidyl]-[4-(trifluoromethoxy)phenyl]methanone;
4-[7-[1-[4-(trifluoromethoxy)benzoyl]-4-piperidyl]-3H-imidazo[4,5-b]pyridin-2-yl]piperidine-4-carbonitrile;
[4-[2-[2-piperidyl]-3H-imidazo[4,5-b]pyridin-7-yl]-1-piperidyl]-[4-(trifluoromethoxy)phenyl]methanone;
[4-[2-(4-aminotetrahydropyran-4-yl)-3H-imidazo[4,5-b]pyridin-7-yl]-1-piperidyl]-[4-(trifluoromethoxy)phenyl]methanone;
[4-[2-(3-amino-4-methoxy-phenyl)-3H-imidazo[4,5-b]pyridin-7-yl]-1-piperidyl]-[4-(trifluoromethoxy)phenyl]methanone;
[4-[2-(2-piperidyl)-3H-imidazo[4,5-b]pyridin-7-yl]-1-piperidyl]-[4-(trifluoromethoxy)phenyl]methanone;
[4-[2-[pyrrolidin-2-yl]-3H-imidazo[4,5-b]pyridin-7-yl]-1-piperidyl]-[4-(trifluoromethoxy)phenyl]methanone;
[4-[2-(1,2,3,4-tetrahydroisoquinolin-6-yl)-3H-imidazo[4,5-b]pyridin-7-yl]-1-piperidyl]-[4-(trifluoromethoxy)phenyl]methanone;
[4-[2-(3-amino-5-hydroxy-phenyl)-3H-imidazo[4,5-b]pyridin-7-yl]-1-piperidyl]-[4-(trifluoromethoxy)phenyl]methanone;
[4-[2-(1H-indol-3-yl)-3H-imidazo[4,5-b]pyridin-7-yl]-1-piperidyl]-[4-(trifluoromethoxy)phenyl]methanone;
[4-[2-(4,5,6,7-tetrahydrothieno[3,2-c]pyridin-2-yl)-3H-imidazo[4,5-b]pyridin-7-yl]-1-piperidyl]-[4-(trifluoromethoxy)phenyl]methanone;
[4-[2-(azetidin-2-yl)-3H-imidazo[4,5-b]pyridin-7-yl]-1-piperidyl]-[4-(trifluoromethoxy)phenyl]methanone;2,2,2-trifluoroacetic acid
[4-[2-(3-oxa-9-azabicyclo[3.3.1]nonan-7-yl)-3H-imidazo[4,5-b]pyridin-7-yl]-1-piperidyl]-[4-(trifluoromethoxy)phenyl]methanone;
[4-[2-(4-amino-3-pyridyl)-3H-imidazo[4,5-b]pyridin-7-yl]-1-piperidyl]-[4-(trifluoromethoxy)phenyl]methanone;
[4-[2-[piperazin-2-yl]-3H-imidazo[4,5-b]pyridin-7-yl]-1-piperidyl]-[4-(trifluoromethoxy)phenyl]methanone;
[4-[2-(3-aminocyclobutyl)-3H-imidazo[4,5-b]pyridin-7-yl]-1-piperidyl]-[4-(trifluoromethoxy)phenyl]methanone;
[4-[2-[3-(methylamino)phenyl]-3H-imidazo[4,5-b]pyridin-7-yl]-1-piperidyl]-[4-(trifluoromethoxy)phenyl]methanone;
[4-[2-(2-azabicyclo[4.1.0]heptan-7-yl)-3H-imidazo[4,5-b]pyridin-7-yl]-1-piperidyl]-[4-(trifluoromethoxy)phenyl]methanone;
[4-[2-[4-hydroxy-6-(trifluoromethyl)-3-quinolyl]-3H-imidazo[4,5-b]pyridin-7-yl]-1-piperidyl]-[4-(trifluoromethoxy)phenyl]methanone;
[4-[2-(3-oxabicyclo[3.1.0]hexan-6-yl)-3H-imidazo[4,5-b]pyridin-7-yl]-1-piperidyl]-[4-(trifluoromethoxy)phenyl]methanone;
N-[4-hydroxy-2-[7-[1-[4-(trifluoromethoxy)benzoyl]-4-piperidyl]-3H-imidazo[4,5-b]pyridin-2-yl]phenyl]acetamide;
5,6-dimethyl-3-[7-[1-[4-(trifluoromethoxy)benzoyl]-4-piperidyl]-3H-imidazo[4,5-b]pyridin-2-yl]-1H-pyridin-2-one;
1-[3,5-dimethyl-4-[7-[1-[4-(trifluoromethoxy)benzoyl]-4-piperidyl]-3H-imidazo[4,5-b]pyridin-2-yl]-1H-pyrrol-2-yl]ethanone;
[4-[2-[1-(1,1-dioxothiolan-3-yl)-4-piperidyl]-3H-imidazo[4,5-b]pyridin-7-yl]-1-piperidyl]-[4-(trifluoromethoxy)phenyl]methanone;
[4-[2-(1-methylsulfonylazetidin-3-yl)-3H-imidazo[4,5-b]pyridin-7-yl]-1-piperidyl]-[4-(trifluoromethoxy)phenyl]methanone;
[2-hydroxy-4-(trifluoromethoxy)phenyl]-[4-(2-tetrahydropyran-4-yl-3H-imidazo[4,5-b]pyridin-7-yl)-1-piperidyl]methanone;
[4-[2-(1,4-oxazepan-2-yl)-3H-imidazo[4,5-b]pyridin-7-yl]-1-piperidyl]-[4-(trifluoromethoxy)phenyl]methanone;
[4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone;
[4-(1,1,2,2-tetrafluoroethoxy)phenyl]-[4-(2-tetrahydropyran-4-yl-3H-imidazo[4,5-b]pyridin-7-yl)-1-piperidyl]methanone;
[4-(1,1,2,2,2-pentafluoroethoxy)phenyl]-[4-(2-tetrahydropyran-4-yl-3H-imidazo[4,5-b]pyridin-7-yl)-1-piperidyl]methanone;
[4-(2-phenyl-3H-imidazo[4,5-b]pyridin-7-yl)-1-piperidyl]-[4-(trifluoromethoxy)phenyl]methanone;
(2,2,3,3-tetrafluoro-1,4-benzodioxin-6-yl)-[4-(2-tetrahydropyran-4-yl-3H-imidazo[4,5-b]pyridin-7-yl)-1-piperidyl]methanone;
[4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[7-oxa-4-azaspiro[2.5]octan-6-yl]-3H-imidazo[4,5-b]pyridin-7-yl]-1-piperidyl]methanone;
[4-[2-[rac-(1S,3S)-3-aminocyclohexyl]-3H-imidazo[4,5-b]pyridin-7-yl]-1-piperidyl]-[4-(trifluoromethoxy)phenyl]methanone;
[4-(2-cyclopentyl-3H-imidazo[4,5-b]pyridin-7-yl)-1-piperidyl]-[4-(trifluoromethoxy)phenyl]methanone;
[4-[2-(4-methylpiperazin-1-yl)-3H-imidazo[4,5-b]pyridin-7-yl]-1-piperidyl]-[4-(trifluoromethoxy)phenyl]methanone;
[4-[2-[7-oxa-4-azaspiro[2.5]octan-6-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-(2-cyclopent-3-en-1-yl-3H-imidazo[4,5-b]pyridin-7-yl)-1-piperidyl]methanone;
[4-[2-(azepan-3-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-(2-cyclopentyl-3H-imidazo[4,5-b]pyridin-7-yl)-1-piperidyl]methanone;
[4-[2-[4-amino-1-methyl-2-oxabicyclo[2.1.1]hexan-3-yl]-3H-imidazo[4,5-b]pyridin-7-yl]-1-piperidyl]-[2-amino-4-(trifluoromethoxy)phenyl]methanone;
[4-[2-(3-hydroxyphenyl)-3H-imidazo[4,5-b]pyridin-7-yl]-1-piperidyl]-[4-(trifluoromethoxy)phenyl]methanone;
[4-[2-[rac-(1R,3S)-3-aminocyclohexyl]-3H-imidazo[4,5-b]pyridin-7-yl]-1-piperidyl]-[4-(trifluoromethoxy)phenyl]methanone;
[4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[4-(2-tetrahydropyran-4-yl-3H-imidazo[4,5-b]pyridin-7-yl)azepan-1-yl]methanone;
[4-[2-(3-fluoro-4-piperidyl)-3H-imidazo[4,5-b]pyridin-7-yl]-1-piperidyl]-[4-(trifluoromethoxy)phenyl]methanone;
[4-[2-(1,2,3,6-tetrahydropyridin-4-yl)-3H-imidazo[4,5-b]pyridin-7-yl]-1-piperidyl]-[4-(trifluoromethoxy)phenyl]methanone;
3-[7-[1-[4-(trifluoromethoxy)benzoyl]-4-piperidyl]-3H-imidazo[4,5-b]pyridin-2-yl]bicyclo[1.1.1]pentane-1-carboxylic acid;
3-[4-(2-tetrahydropyran-4-yl-3H-imidazo[4,5-b]pyridin-7-yl)piperidine-1-carbonyl]-6-(trifluoromethyl)-1H-pyridin-2-one;
[4-(difluoromethoxy)phenyl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[1,1-dioxothiolan-3-yl]-3H-imidazo[4,5-b]pyridin-7-yl]-1-piperidyl]methanone;
[4-[2-[1,4-oxazepan-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[1,4-oxazepan-2-yl]-3H-imidazo[4,5-b]pyridin-7-yl]-1-piperidyl]methanone;
[4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[4-(2-tetrahydropyran-4-yl-3H-imidazo[4,5-b]pyridin-7-yl)azepan-1-yl]methanone;
(trans)-4-[7-[1-[4-(trifluoromethoxy)benzoyl]-4-piperidyl]-3H-imidazo[4,5-b]pyridin-2-yl]cyclohexanecarboxylic acid;
(cis)-[4-[2-[3-aminotetrahydrofuran-2-yl]-3H-imidazo[4,5-b]pyridin-7-yl]-1-piperidyl]-[2-amino-4-(trifluoromethoxy)phenyl]methanone;
[4-[2-(1,1-dioxothiolan-3-yl)-3H-imidazo[4,5-b]pyridin-7-yl]-1-piperidyl]-[4-(trifluoromethoxy)phenyl]methanone;
[4-[2-(4-hydroxy-4-methyl-cyclohexyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(trifluoromethoxy)phenyl]-[4-[2-(4-hydroxy-4-methyl-cyclohexyl)-3H-imidazo[4,5-b]pyridin-7-yl]-1-piperidyl]methanone;
[2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(trifluoromethoxy)phenyl]-[4-[2-(1,2,3,6-tetrahydropyridin-4-yl)-3H-imidazo[4,5-b]pyridin-7-yl]-1-piperidyl]methanone;
[4-[2-[azepan-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[azepan-3-yl]-3H-imidazo[4,5-b]pyridin-7-yl]-1-piperidyl]methanone;
[2-amino-4-(pentafluoro-λ⁶-sulfanyl)phenyl]-[4-(2-cyclopentyl-3H-imidazo[4,5-b]pyridin-7-yl)-1-piperidyl]methanone;
[4-(2-cyclopentyl-3H-imidazo[4,5-b]pyridin-7-yl)-1-piperidyl]-[4-(pentafluoro-λ6-sulfanyl)phenyl]methanone;
[2-amino-4-(pentafluoro-λ⁶-sulfanyl)phenyl]-[4-(2-tetrahydrofuran-2-yl-3H-imidazo[4,5-b]pyridin-7-yl)-1-piperidyl]methanone;
(cis)-[4-[2-(4-methoxycyclohexyl)-3H-imidazo[4,5-b]pyridin-7-yl]-1-piperidyl]-[4-(pentafluoro-λ⁶-sulfanyl)phenyl]methanone;
(trans)-[4-[2-(4-methoxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
(cis)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone;
(trans)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone;
[4-(pentafluoro-λ⁶-sulfanyl)phenyl]-[4-[2-[tetrahydrofuran-2-yl]-3H-imidazo[4,5-b]pyridin-7-yl]-1-piperidyl]methanone;
[4-(pentafluoro-λ⁶-sulfanyl)phenyl]-[4-[2-[tetrahydrofuran-3-yl]-3H-imidazo[4,5-b]pyridin-7-yl]-1-piperidyl]methanone;
[4-[2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[4-(pentafluoro-λ⁶-sulfanyl)phenyl]-[4-[2-(4-piperidyl)-3H-imidazo[4,5-b]pyridin-7-yl]-1-piperidyl]methanone;hydrochloride;
[4-[2-[morpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(3-methoxy-1-bicyclo[1.1.1]pentanyl)-3H-imidazo[4,5-b]pyridin-7-yl]-1-piperidyl]methanone;
[4-(2-N-morpholino-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-(4-piperidyl)-3H-imidazo[4,5-b]pyridin-7-yl]-1-piperidyl]methanone;
[2-amino-4-(pentafluoro-λ⁶-sulfanyl)phenyl]-[4-[2-[tetrahydrofuran-3-yl]-3H-imidazo[4,5-b]pyridin-7-yl]-1-piperidyl]methanone;
(cis)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone;
(trans)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone;
[2-amino-4-(pentafluoro-λ⁶-sulfanyl)phenyl]-[4-(2-N-morpholino-3H-imidazo[4,5-b]pyridin-7-yl)-1-piperidyl]methanone;
(trans)-[4-[2-(3-methoxycyclobutyl)-3H-imidazo[4,5-b]pyridin-7-yl]-1-piperidyl]-[4-(pentafluoro-λ⁶-sulfanyl)phenyl]methanone;
[4-[2-(1-methyl-4-piperidyl)-3H-imidazo[4,5-b]pyridin-7-yl]-1-piperidyl]-[4-(pentafluoro-λ⁶-sulfanyl)phenyl]methanone;
[4-[2-(1-cyclopropyl-4-piperidyl)-3H-imidazo[4,5-b]pyridin-7-yl]-1-piperidyl]-[4-(pentafluoro-λ⁶-sulfanyl)phenyl]methanone;
[4-[2-[morpholin-2-yl]-3H-imidazo[4,5-b]pyridin-7-yl]-1-piperidyl]-[4-(pentafluoro-λ⁶-sulfanyl)phenyl]methanone;
(cis)-[4-[2-(3-methoxycyclobutyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(1-methyl-4-piperidyl)-3H-imidazo[4,5-b]pyridin-7-yl]-1-piperidyl]methanone;
[2-amino-4-(pentafluoro-λ⁶-sulfanyl)phenyl]-[4-[2-[morpholin-2-yl]-3H-imidazo[4,5-b]pyridin-7-yl]-1-piperidyl]methanone;
[2-amino-4-(pentafluoro-λ⁶-sulfanyl)phenyl]-[4-[2-(1-cyclopropyl-4-piperidyl)-3H-imidazo[4,5-b]pyridin-7-yl]-1-piperidyl]methanone;
[4-[2-(1-methoxy-1-methyl-ethyl)-3H-imidazo[4,5-b]pyridin-7-yl]-1-piperidyl]-[4-(pentafluoro-λ⁶-sulfanyl)phenyl]methanone;
[2-(methylamino)-4-(pentafluoro-λ⁶-sulfanyl)phenyl]-[4-[2-(1-methyl-4-piperidyl)-3H-imidazo[4,5-b]pyridin-7-yl]-1-piperidyl]methanone;
[2-amino-4-(pentafluoro-λ⁶-sulfanyl)phenyl]-[4-[2-(1-methoxy-1-methyl-ethyl)-3H-imidazo[4,5-b]pyridin-7-yl]-1-piperidyl]methanone;
(cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
[4-[2-[4-methylmorpholin-2-yl]-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
(cis)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[morpholin-2-yl]-3H-imidazo[4,5-b]pyridin-7-yl]-1-piperidyl]methanone;
(trans)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone;
[4-[2-[4-methylmorpholin-2-yl]-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone;
[4-(pentafluoro-λ⁶-sulfanyl)phenyl]-[4-(2-tetrahydropyran-4-yl-3H-imidazo[4,5-b]pyridin-7-yl)azepan-1-yl]methanone;
[4-[2-[2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone;
3-methyl-3-[7-[1-[4-(pentafluoro-λ⁶-sulfanyl)benzoyl]-4-piperidyl]-3H-imidazo[4,5-b]pyridin-2-yl]cyclobutanone;
[4-[2-(1-hydroxy-1-methyl-ethyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(1-hydroxy-1-methyl-ethyl)-3H-imidazo[4,5-b]pyridin-7-yl]-1-piperidyl]methanone;
[(cis)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[(trans)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-[2-oxa-5-azabicyclo[2.2.1]heptan-1-yl]-3H-imidazo[4,5-b]pyridin-7-yl]-1-piperidyl]methanone;
[2-amino-4-(trifluoromethoxy)phenyl]-[4-fluoro-4-(6-fluoro-2-tetrahydropyran-4-yl-3H-imidazo[4,5-b]pyridin-7-yl)-1-piperidyl]methanone;
[4-[2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(3-hydroxy-1-bicyclo[1.1.1]pentanyl)-3H-imidazo[4,5-b]pyridin-7-yl]-1-piperidyl]methanone;
[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[2-hydroxy-4-(pentafluoro-λ⁶-sulfanyl)phenyl]-[4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[4-(6-fluoro-2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(cis)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(trans)-2-methyl-4-(2-tetrahydropyran-4-yl-3H-imidazo[4,5-b]pyridin-7-yl)-1-piperidyl]methanone;
(cis)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone;
(trans)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone;
[4-(6-fluoro-2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(6-fluoro-2-morpholin-2-yl-3H-imidazo[4,5-b]pyridin-7-yl)-1-piperidyl]-[4-(pentafluoro-λ⁶-sulfanyl)phenyl]methanone;
[4-[6-fluoro-2-(2-oxabicyclo[2.2.2]octan-4-yl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(2-oxabicyclo[2.2.2]octan-4-yl)-3H-imidazo[4,5-b]pyridin-7-yl]-1-piperidyl]methanone;
[4-(6-fluoro-2-tetrahydropyran-3-yl-3H-imidazo[4,5-b]pyridin-7-yl)-1-piperidyl]-[4-(pentafluoro-λ⁶-sulfanyl)phenyl]methanone;
(cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
[2-fluoro-4-(pentafluoro-λ⁶-sulfanyl)phenyl]-[4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[4-(6-fluoro-2-tetrahydropyran-3-yl-3H-imidazo[4,5-b]pyridin-7-yl)-1-piperidyl]methanone;
(trans)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone;
(cis)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone;
[4-(6-fluoro-2-tetrahydrofuran-3-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(oxepan-4-yl)-3H-imidazo[4,5-b]pyridin-7-yl]-1-piperidyl]methanone;
[4-[6-fluoro-2-(oxepan-4-yl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(6-fluoro-2-tetrahydrofuran-3-yl-3H-imidazo[4,5-b]pyridin-7-yl)-1-piperidyl]methanone;
[4-[2-[6-methylmorpholin-2-yl]-3H-imidazo[4,5-b]pyridin-7-yl]-1-piperidyl]-[4-(pentafluoro-λ⁶-sulfanyl)phenyl]methanone;
[4-[6-fluoro-2-(1-methoxy-1-methyl-ethyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(pentafluoro-λ⁶-sulfanyl)phenyl]-[4-[6-fluoro-2-(3-methoxy-1-bicyclo[1.1.1]pentanyl)-3H-imidazo[4,5-b]pyridin-7-yl]-1-piperidyl]methanone;
[4-[6-fluoro-2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(6-fluoro-2-tetrahydrofuran-2-yl-3H-imidazo[4,5-b]pyridin-7-yl)-1-piperidyl]methanone;
[4-(6-fluoro-2-tetrahydrofuran-2-yl-3H-imidazo[4,5-b]pyridin-7-yl)-1-piperidyl]-[4-(pentafluoro-λ⁶-sulfanyl)phenyl]methanone;
[4-(6-fluoro-2-tetrahydropyran-4-yl-3H-imidazo[4,5-b]pyridin-7-yl)-1-piperidyl]-[2-hydroxy-4-(pentafluoro-λ⁶-sulfanyl)phenyl]methanone;
[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone;
[2-amino-4-(pentafluoro-λ⁶-sulfanyl)phenyl]-[4-[2-[4-(hydroxymethyl)-1-piperidyl]-3H-imidazo[4,5-b]pyridin-7-yl]-1-piperidyl]methanone;
[4-[6-fluoro-2-(3-methoxycyclobutyl)-3H-imidazo[4,5-b]pyridin-7-yl]-1-piperidyl]-[4-(pentafluoro-λ⁶-sulfanyl)phenyl]methanone;
[4-(6-fluoro-2-tetrahydropyran-2-yl-3H-imidazo[4,5-b]pyridin-7-yl)-1-piperidyl]-[4-(pentafluoro-λ⁶-sulfanyl)phenyl]methanone;
(trans)-[4-[6-fluoro-2-(4-hydroxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
[4-[6-fluoro-2-[2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
[4-[2-[3-methoxycyclohexyl]-3H-imidazo[4,5-b]pyridin-7-yl]-1-piperidyl]-[4-(pentafluoro-λ⁶-sulfanyl)phenyl]methanone;
[4-(6-fluoro-2-thiomorpholin-2-yl-3H-imidazo[4,5-b]pyridin-7-yl)-1-piperidyl]-[4-(pentafluoro-λ⁶-sulfanyl)phenyl]methanone;
(trans)-[4-[6-fluoro-2-(4-methoxycyclohexyl)-3H-imidazo[4,5-b]pyridin-7-yl]-1-piperidyl]-[2-hydroxy-4-(pentafluoro-λ⁶-sulfanyl)phenyl]methanone;
[2-amino-4-(pentafluoro-λ⁶-sulfanyl)phenyl]-[4-[6-fluoro-2-(1-methoxy-1-methyl-ethyl)-3H-imidazo[4,5-b]pyridin-7-yl]-1-piperidyl]methanone;
[4-[6-fluoro-2-[tetrahydrofuran-3-yl]-3H-imidazo[4,5-b]pyridin-7-yl]-1-piperidyl]-[4-(pentafluoro-λ⁶-sulfanyl)phenyl]methanone;
[4-[6-fluoro-2-[6-methylmorpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[tetrahydrofuran-3-yl]-3H-imidazo[4,5-b]pyridin-7-yl]-1-piperidyl]methanone;
[4-[6-fluoro-2-[morpholin-2-yl]-3H-imidazo[4,5-b]pyridin-7-yl]-1-piperidyl]-[4-(pentafluoro-λ⁶-sulfanyl)phenyl]methanone;
5-[6-fluoro-7-[1-[4-(pentafluoro-λ⁶-sulfanyl)benzoyl]-4-piperidyl]-3H-imidazo[4,5-b]pyridin-2-yl]piperidin-2-one;
(trans)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(2-cyclopentyl-6-fluoro-3H-imidazo[4,5-b]pyridin-7-yl)-1-piperidyl]methanone;
[2-amino-4-(pentafluoro-λ⁶-sulfanyl)phenyl]-[4-[2-[3-methoxycyclohexyl]-3H-imidazo[4,5-b]pyridin-7-yl]-1-piperidyl]methanone;
[4-[6-fluoro-2-[oxepan-4-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[oxepan-4-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[tetrahydrofuran-2-yl]-3H-imidazo[4,5-b]pyridin-7-yl]-1-piperidyl]methanone;
[4-[6-fluoro-2-[tetrahydrofuran-2-yl]-3H-imidazo[4,5-b]pyridin-7-yl]-1-piperidyl]-[4-(pentafluoro-λ⁶-sulfanyl)phenyl]methanone;
[4-[6-fluoro-2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(3-hydroxy-1-bicyclo[1.1.1]pentanyl)-3H-imidazo[4,5-b]pyridin-7-yl]-1-piperidyl]methanone;
(trans)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
[4-(2-cyclopentyl-6-fluoro-3H-imidazo[4,5-b]pyridin-7-yl)-1-piperidyl]-[4-(pentafluoro-λ⁶-sulfanyl)phenyl]methanone;
[4-[6-fluoro-2-(3-methoxy-1-bicyclo[1.1.1]pentanyl)-3H-imidazo[4,5-b]pyridin-7-yl]-1-piperidyl]-[4-(trifluoromethoxy)phenyl]methanone;
[4-[6-fluoro-2-(3-methoxy-1-bicyclo[1.1.1]pentanyl)-3H-imidazo[4,5-b]pyridin-7-yl]-1-piperidyl]-[2-hydroxy-4-(pentafluoro-λ⁶-sulfanyl)phenyl]methanone;
[2-amino-4-(trifluoromethoxy)phenyl]-[4-[6-fluoro-2-(3-methoxy-1-bicyclo[1.1.1]pentanyl)-3H-imidazo[4,5-b]pyridin-7-yl]-1-piperidyl]methanone;
[4-[6-fluoro-2-(2-oxabicyclo[2.2.2]octan-4-yl)-3H-imidazo[4,5-b]pyridin-7-yl]-1-piperidyl]-[4-(trifluoromethoxy)phenyl]methanone;
[2-amino-5-(pentafluoro-λ⁶-sulfanyl)phenyl]-[4-(2-tetrahydropyran-4-yl-3H-imidazo[4,5-b]pyridin-7-yl)-1-piperidyl]methanone;
[4-[2-(1-cyclopropyl-4-piperidyl)-6-fluoro-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-[3-(trifluoromethyl)-1-bicyclo[1.1.1]pentanyl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[tetrahydropyran-2-yl]-3H-imidazo[4,5-b]pyridin-7-yl]-1-piperidyl]methanone;
[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;
[4-[6-fluoro-2-[3-(trifluoromethyl)-1-bicyclo[1.1.1]pentanyl]-3H-imidazo[4,5-b]pyridin-7-yl]-1-piperidyl]-[4-(trifluoromethoxy)phenyl]methanone;
[4-[2-(1-methoxy-1-methyl-ethyl)-3H-imidazo[4,5-b]pyridin-7-yl]-1-piperidyl]-[4-(trifluoromethoxy)phenyl]methanone;
[4-[6-fluoro-2-[tetrahydropyran-2-yl]-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone;
(cis)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone;
(cis)-[4-[6-fluoro-2-(4-methoxycyclohexyl)-3H-imidazo[4,5-b]pyridin-7-yl]-1-piperidyl]-[4-(trifluoromethoxy)phenyl]methanone;
(trans)-[4-[6-fluoro-2-(4-methoxycyclohexyl)-3H-imidazo[4,5-b]pyridin-7-yl]-1-piperidyl]-[4-(trifluoromethoxy)phenyl]methanone;
[4-[6-fluoro-2-(1-methoxy-1-methyl-ethyl)-3H-imidazo[4,5-b]pyridin-7-yl]-1-piperidyl]-[4-(trifluoromethoxy)phenyl]methanone;
(trans)-(4-(6-fluoro-2-(4-hydroxy-4-methylcyclohexyl)-3H-imidazo[4,5-b]pyridin-7-yl)piperidin-1-yl)(4-(pentafluoro-λ⁶-sulfanyl)phenyl)methanone;
(cis)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-(1-methoxy-1-methyl-ethyl)-3H-imidazo[4,5-b]pyridin-7-yl]-1-piperidyl]methanone;
(trans)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone;
4-(6-fluoro-2-N-morpholino-3H-imidazo[4,5-b]pyridin-7-yl)-1-piperidyl]-[4-(pentafluoro-λ⁶-sulfanyl)phenyl]methanone;
(trans)-[4-[6-fluoro-2-[4-hydroxy-4-(trifluoromethyl)cyclohexyl]-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-(2-oxabicyclo[2.2.2]octan-4-yl)-3H-imidazo[4,5-b]pyridin-7-yl]-1-piperidyl]methanone;
[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
(cis)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
2-[6-fluoro-7-[1-[4-(pentafluoro-λ⁶-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylic acid;
[4-[2-(1,1-dioxo-1,4-thiazinan-2-yl)-6-fluoro-3H-imidazo[4,5-b]pyridin-7-yl]-1-piperidyl]-[4-(pentafluoro-λ⁶-sulfanyl)phenyl]methanone;
[4-[6-fluoro-2-(3-methoxycyclobutyl)-3H-imidazo[4,5-b]pyridin-7-yl]-1-piperidyl]-[4-(trifluoromethoxy)phenyl]methanone;
(2-amino-4-(trifluoromethoxy) phenyl]-[4-[6-fluoro-2-(3-methoxycyclobutyl)-3H-imidazo[4,5-b]pyridin-7-yl]-1-piperidyl]methanone;
(cis)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone;
(trans)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone; and
7-[6-fluoro-7-[1-[4-(trifluoromethoxy)benzoyl]-4-piperidyl]-3H-imidazo[4,5-b]pyridin-2-yl]-2-oxa-5-azaspiro[3.5]nonane-5-carboxylic acid,
   and the pharmaceutically acceptable salts thereof.

In embodiments where the compound has enantiomeric forms (e.g., where the compound has a chiral centre, such as a chiral carbon atom), the compound is present as a racemic mixture of enantiomers. In embodiments where the compound has a chiral centre (e.g., a chiral carbon atom), the compound is present as the (R) isomer. In other embodiments where the compound has a chiral centre (e.g., a chiral carbon atom), the compound is present as the (S) isomer. Thus, in embodiments, the compound is selected from the group consisting of:
(R)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
(S)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
(R)-[2-amino-4-(trifluoromethoxy)phenyl]-[4-(2-tetrahydrofuran-3-yl-3H-imidazo[4,5-b]pyridin-7-yl)-1-piperidyl]methanone,
(S)-[2-amino-4-(trifluoromethoxy)phenyl]-[4-(2-tetrahydrofuran-3-yl-3H-imidazo[4,5-b]pyridin-7-yl)-1-piperidyl]methanone,
(rac)-5-[7-[1-[4-(trifluoromethoxy)benzoyl]-4-piperidyl]-3H-imidazo[4,5-b]pyridin-2-yl]piperidin-2-one,
(rac)-[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone,
(R)-[4-(2-morpholin-2-yl-3H-imidazo[4,5-b]pyridin-7-yl]-1-piperidyl]-[4-(trifluoromethoxy)phenyl]methanone,
(S)-[4-(2-morpholin-2-yl-3H-imidazo[4,5-b]pyridin-7-yl]-1-piperidyl]-[4-(trifluoromethoxy)phenyl]methanone,
(R)-[4-[2-(1-methylpyrrolidin-3-yl)-3H-imidazo[4,5-b]pyridin-7-yl]-1-piperidyl]-[4-(trifluoromethoxy)phenyl]methanone,
(S)-[4-[2-(1-methylpyrrolidin-3-yl)-3H-imidazo[4,5-b]pyridin-7-yl]-1-piperidyl]-[4-(trifluoromethoxy)phenyl]methanone,
(R)-[4-(2-tetrahydrofuran-3-yl-3H-imidazo[4,5-b]pyridin-7-yl)-1-piperidyl]-[4-(trifluoromethoxy)phenyl]methanone,
(S)-[4-(2-tetrahydrofuran-3-yl-3H-imidazo[4,5-b]pyridin-7-yl)-1-piperidyl]-[4-(trifluoromethoxy)phenyl]methanone,
(rac)-[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone,
(rac)-[2-amino-4-(trifluoromethoxy)phenyl]-[4-(2-morpholin-2-yl-3H-imidazo[4,5-b]pyridin-7-yl)-1-piperidyl]methanone,
(rac)-[4-[2-(2,2-dimethyltetrahydropyran-4-yl)-3H-imidazo[4,5-b]pyridin-7-yl]-1-piperidyl]-[4-(trifluoromethoxy)phenyl]methanone,
(rac)-[4-[2-(6,6-dimethyltetrahydropyran-3-yl)-3H-imidazo[4,5-b]pyridin-7-yl]-1-piperidyl]-[4-(trifluoromethoxy)phenyl]methanone,
(R)-[4-[2-(1,4-dioxan-2-yl)-3H-imidazo[4,5-b]pyridin-7-yl]-1-piperidyl]-[4-(trifluoromethoxy)phenyl]methanone,
(S)-[4-[2-(1,4-dioxan-2-yl)-3H-imidazo[4,5-b]pyridin-7-yl]-1-piperidyl]-[4-(trifluoromethoxy)phenyl]methanone,
(rac)-[4-(2-tetrahydrofuran-2-yl-3H-imidazo[4,5-b]pyridin-7-yl)-1-piperidyl]-[4-(trifluoromethoxy)phenyl]methanone,
(rac)-[4-[2-(4-methylmorpholin-2-yl)-3H-imidazo[4,5-b]pyridin-7-yl]-1-piperidyl]-[4-(trifluoromethoxy)phenyl]methanone,
(rac)-[2-amino-4-(trifluoromethoxy)phenyl]-[4-[2-(5,5-difluoro-3-piperidyl)-3H-imidazo[4,5-b]pyridin-7-yl]-1-piperidyl]methanone,
(rac)-[4-[2-(tetrahydrofuran-3-ylmethyl)-3H-imidazo[4,5-b]pyridin-7-yl]-1-piperidyl]-[4-(trifluoromethoxy)phenyl]methanone,
(rac)-[4-[2-(oxepan-4-yl)-3H-imidazo[4,5-b]pyridin-7-yl]-1-piperidyl]-[4-(trifluoromethoxy)phenyl]methanone,
(rac)-[2-amino-4-(trifluoromethoxy)phenyl]-[4-[2-(oxepan-4-yl)-3H-imidazo[4,5-b]pyridin-7-yl]-1-piperidyl]methanone,
(rac)-[4-(2-tetrahydropyran-2-yl-3H-imidazo[4,5-b]pyridin-7-yl)-1-piperidyl]-[4-(trifluoromethoxy)phenyl]methanone,
(rac)-[2-amino-4-(trifluoromethoxy)phenyl]-[4-(2-tetrahydropyran-2-yl-3H-imidazo[4,5-b]pyridin-7-yl)-1-piperidyl]methanone,
(rac)-[4-[2-(5,5-difluoro-3-piperidyl)-3H-imidazo[4,5-b]pyridin-7-yl]-1-piperidyl]-[4-(trifluoromethoxy)phenyl]methanone,
(rac)-[4-(2-pyrrolidin-3-yl-3H-imidazo[4,5-b]pyridin-7-yl)-1-piperidyl]-[4-(trifluoromethoxy)phenyl]methanone,
(rac)-[2-amino-4-(trifluoromethoxy)phenyl]-[4-(2-tetrahydropyran-3-yl-3H-imidazo[4,5-b]pyridin-7-yl)-1-piperidyl]methanone, and
(rac)-[4-(2-tetrahydropyran-3-yl-3H-imidazo[4,5-b]pyridin-7-yl)-1-piperidyl]-[4-(trifluoromethoxy)phenyl]methanone,
   and the pharmaceutically acceptable salts thereof.

In embodiments the compound is selected from the group consisting of:
(R)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
(S)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
(rac)-[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone,
(R)-[4-[2-(1-methylpyrrolidin-3-yl)-3H-imidazo[4,5-b]pyridin-7-yl]-1-piperidyl]-[4-(trifluoromethoxy)phenyl]methanone,
(S)-[4-[2-(1-methylpyrrolidin-3-yl)-3H-imidazo[4,5-b]pyridin-7-yl]-1-piperidyl]-[4-(trifluoromethoxy)phenyl]methanone,
(R)-[4-(2-tetrahydrofuran-3-yl-3H-imidazo[4,5-b]pyridin-7-yl)-1-piperidyl]-[4-(trifluoromethoxy)phenyl]methanone,
(S)-[4-(2-tetrahydrofuran-3-yl-3H-imidazo[4,5-b]pyridin-7-yl)-1-piperidyl]-[4-(trifluoromethoxy)phenyl]methanone,
(rac)-[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone,
(rac)-[4-[2-(2,2-dimethyltetrahydropyran-4-yl)-3H-imidazo[4,5-b]pyridin-7-yl]-1-piperidyl]-[4-(trifluoromethoxy)phenyl]methanone,
(rac)-[4-[2-(6,6-dimethyltetrahydropyran-3-yl)-3H-imidazo[4,5-b]pyridin-7-yl]-1-piperidyl]-[4-(trifluoromethoxy)phenyl]methanone,
(R)-[4-[2-(1,4-dioxan-2-yl)-3H-imidazo[4,5-b]pyridin-7-yl]-1-piperidyl]-[4-(trifluoromethoxy)phenyl]methanone,
(S)-[4-[2-(1,4-dioxan-2-yl)-3H-imidazo[4,5-b]pyridin-7-yl]-1-piperidyl]-[4-(trifluoromethoxy)phenyl]methanone,
(rac)-[4-(2-tetrahydrofuran-2-yl-3H-imidazo[4,5-b]pyridin-7-yl)-1-piperidyl]-[4-(trifluoromethoxy)phenyl]methanone,
(rac)-[4-[2-(4-methylmorpholin-2-yl)-3H-imidazo[4,5-b]pyridin-7-yl]-1-piperidyl]-[4-(trifluoromethoxy)phenyl]methanone,
(rac)-[2-amino-4-(trifluoromethoxy)phenyl]-[4-[2-(5,5-difluoro-3-piperidyl)-3H-imidazo[4,5-b]pyridin-7-yl]-1-piperidyl]methanone,
(rac)-[4-[2-(tetrahydrofuran-3-ylmethyl)-3H-imidazo[4,5-b]pyridin-7-yl]-1-piperidyl]-[4-(trifluoromethoxy)phenyl]methanone,
(rac)-[4-[2-(oxepan-4-yl)-3H-imidazo[4,5-b]pyridin-7-yl]-1-piperidyl]-[4-(trifluoromethoxy)phenyl]methanone,
(rac)-[2-amino-4-(trifluoromethoxy)phenyl]-[4-[2-(oxepan-4-yl)-3H-imidazo[4,5-b]pyridin-7-yl]-1-piperidyl]methanone,
(rac)-[4-(2-tetrahydropyran-2-yl-3H-imidazo[4,5-b]pyridin-7-yl)-1-piperidyl]-[4-(trifluoromethoxy)phenyl]methanone,
(rac)-[2-amino-4-(trifluoromethoxy)phenyl]-[4-(2-tetrahydropyran-2-yl-3H-imidazo[4,5-b]pyridin-7-yl)-1-piperidyl]methanone,
(rac)-[4-[2-(5,5-difluoro-3-piperidyl)-3H-imidazo[4,5-b]pyridin-7-yl]-1-piperidyl]-[4-(trifluoromethoxy)phenyl]methanone,
(rac)-[4-(2-pyrrolidin-3-yl-3H-imidazo[4,5-b]pyridin-7-yl)-1-piperidyl]-[4-(trifluoromethoxy)phenyl]methanone,
(rac)-[2-amino-4-(trifluoromethoxy)phenyl]-[4-(2-tetrahydropyran-3-yl-3H-imidazo[4,5-b]pyridin-7-yl)-1-piperidyl]methanone, and
(rac)-[4-(2-tetrahydropyran-3-yl-3H-imidazo[4,5-b]pyridin-7-yl)-1-piperidyl]-[4-(trifluoromethoxy)phenyl]methanone,
   and the pharmaceutically acceptable salts thereof.

In embodiments the compound is selected from the group consisting of:
[4-[2-[(2R)-tetrahydrofuran-2-yl]-3H-imidazo[4,5-b]pyridin-7-yl]-1-piperidyl]-[4-(trifluoromethoxy)phenyl]methanone;
[4-[2-[(2S)-tetrahydrofuran-2-yl]-3H-imidazo[4,5-b]pyridin-7-yl]-1-piperidyl]-[4-(trifluoromethoxy)phenyl]methanone;
[4-[2-[(3R)-pyrrolidin-3-yl]-3H-imidazo[4,5-b]pyridin-7-yl]-1-piperidyl]-[4-(trifluoromethoxy)phenyl]methanone;
[4-[2-[(3S)-pyrrolidin-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(2R)-tetrahydropyran-2-yl]-3H-imidazo[4,5-b]pyridin-7-yl]-1-piperidyl]methanone;
[2-amino-4-(trifluoromethoxy)phenyl]-[4-[2-[(2S)-tetrahydropyran-2-yl]-3H-imidazo[4,5-b]pyridin-7-yl]-1-piperidyl]methanone;
[4-[2-[(3R)-5,5-difluoro-3-piperidyl]-3H-imidazo[4,5-b]pyridin-7-yl]-1-piperidyl]-[4-(trifluoromethoxy)phenyl]methanone;
[4-[2-[(3S)-5,5-difluoro-3-piperidyl]-3H-imidazo[4,5-b]pyridin-7-yl]-1-piperidyl]-[4-(trifluoromethoxy)phenyl]methanone;
(rac)-[2-amino-4-(trifluoromethoxy)phenyl]-[4-[2-(1,4-dioxan-2-yl)-3H-imidazo[4,5-b]pyridin-7-yl]-1-piperidyl]methanone;
(rac)-[3-(2-tetrahydropyran-4-yl-3H-imidazo[4,5-b]pyridin-7-yl)pyrrolidin-1-yl]-[4-(trifluoromethoxy)phenyl]methanone;
(rac)-[2-amino-4-(trifluoromethoxy)phenyl]-[3-(2-tetrahydropyran-4-yl-3H-imidazo[4,5-b]pyridin-7-yl)pyrrolidin-1-yl]methanone;
[2-amino-4-(trifluoromethoxy)phenyl]-[4-[2-[(2R)-1,4-dioxan-2-yl]-3H-imidazo[4,5-b]pyridin-7-yl]-1-piperidyl]methanone;
[2-amino-4-(trifluoromethoxy)phenyl]-[4-[2-[(2S)-1,4-dioxan-2-yl]-3H-imidazo[4,5-b]pyridin-7-yl]-1-piperidyl]methanone;
[2-amino-4-(trifluoromethoxy)phenyl]-[4-[2-[(3R)-5,5-difluoro-3-piperidyl]-3H-imidazo[4,5-b]pyridin-7-yl]-1-piperidyl]methanone;
[2-amino-4-(trifluoromethoxy)phenyl]-[4-[2-[(3S)-5,5-difluoro-3-piperidyl]-3H-imidazo[4,5-b]pyridin-7-yl]-1-piperidyl]methanone;
[2-amino-4-(trifluoromethoxy)phenyl]-[(3R)-3-(2-tetrahydropyran-4-yl-3H-imidazo[4,5-b]pyridin-7-yl)pyrrolidin-1-yl]methanone;
[2-amino-4-(trifluoromethoxy)phenyl]-[(3S)-3-(2-tetrahydropyran-4-yl-3H-imidazo[4,5-b]pyridin-7-yl)pyrrolidin-1-yl]methanone;
[2-amino-4-(trifluoromethoxy)phenyl]-[4-[2-[(4R)-oxepan-4-yl]-3H-imidazo[4,5-b]pyridin-7-yl]-1-piperidyl]methanone;
[2-amino-4-(trifluoromethoxy)phenyl]-[4-[2-[(4S)-oxepan-4-yl]-3H-imidazo[4,5-b]pyridin-7-yl]-1-piperidyl]methanone;
[(3S)-3-(2-tetrahydropyran-4-yl-3H-imidazo[4,5-b]pyridin-7-yl)pyrrolidin-1-yl]-[4-(trifluoromethoxy)phenyl]methanone;
[(3R)-3-(2-tetrahydropyran-4-yl-3H-imidazo[4,5-b]pyridin-7-yl)pyrrolidin-1-yl]-[4-(trifluoromethoxy)phenyl]methanone;
(rac)-[2-amino-4-(trifluoromethoxy)phenyl]-[4-[2-(2-methylmorpholin-2-yl)-3H-imidazo[4,5-b]pyridin-7-yl]-1-piperidyl]methanone;
[4-[2-[(2S)-6,6-dimethylmorpholin-2-yl]-3H-imidazo[4,5-b]pyridin-7-yl]-1-piperidyl]-[4-(trifluoromethoxy)phenyl]methanone;
[4-[2-[(2R)-6,6-dimethylmorpholin-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(2R)-tetrahydrofuran-2-yl]-3H-imidazo[4,5-b]pyridin-7-yl]-1-piperidyl]methanone;
[2-amino-4-(trifluoromethoxy)phenyl]-[4-[2-[(2S)-tetrahydrofuran-2-yl]-3H-imidazo[4,5-b]pyridin-7-yl]-1-piperidyl]methanone;
(rac)-[4-[2-(7-oxa-4-azaspiro[2.5]octan-6-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(2S)-6,6-dimethylmorpholin-2-yl]-3H-imidazo[4,5-b]pyridin-7-yl]-1-piperidyl]methanone;
[2-amino-4-(trifluoromethoxy)phenyl]-[4-[2-[(2R)-6,6-dimethylmorpholin-2-yl]-3H-imidazo[4,5-b]pyridin-7-yl]-1-piperidyl]methanone;
[2-amino-4-(trifluoromethoxy)phenyl]-[4-[2-[(3R)-tetrahydrofuran-3-yl]-3H-imidazo[4,5-b]pyridin-7-yl]-1-piperidyl]methanone;
[2-amino-4-(trifluoromethoxy)phenyl]-[4-[2-[(3S)-tetrahydrofuran-3-yl]-3H-imidazo[4,5-b]pyridin-7-yl]-1-piperidyl]methanone;
(rac)-[4-[2-(4-amino-1-methyl-2-oxabicyclo[2.1.1]hexan-3-yl)-3H-imidazo[4,5-b]pyridin-7-yl]-1-piperidyl]-[2-amino-4-(trifluoromethoxy)phenyl]methanone;
(rac)-[2-amino-4-(trifluoromethoxy)phenyl]-[4-[2-(7-oxa-4-azaspiro[2.5]octan-6-yl)-3H-imidazo[4,5-b]pyridin-7-yl]-1-piperidyl]methanone;
(rac)-[4-[2-(4-amino-1-methyl-2-oxabicyclo[2.1.1]hexan-3-yl)-3H-imidazo[4,5-b]pyridin-7-yl]-1-piperidyl]-[4-(trifluoromethoxy)phenyl]methanone;
[4-[2-[(3R,4S)-4-phenylpyrrolidin-3-yl]-3H-imidazo[4,5-b]pyridin-7-yl]-1-piperidyl]-[4-(trifluoromethoxy)phenyl]methanone;
[4-[2-[(2R,4S)-4-fluoropyrrolidin-2-yl]-3H-imidazo[4,5-b]pyridin-7-yl]-1-piperidyl]-[4-(trifluoromethoxy)phenyl]methanone;
(rac)-[4-[2-(2-amino-3,3,3-trifluoro-propyl)-3H-imidazo[4,5-b]pyridin-7-yl]-1-piperidyl]-[4-(trifluoromethoxy)phenyl]methanone;
[4-[2-[(2S)-pyrrolidin-2-yl]-3H-imidazo[4,5-b]pyridin-7-yl]-1-piperidyl]-[4-(trifluoromethoxy)phenyl]methanone;
[4-[2-[(2R)-2-piperidyl]-3H-imidazo[4,5-b]pyridin-7-yl]-1-piperidyl]-[4-(trifluoromethoxy)phenyl]methanone;
(rac)-[4-[2-(2-piperidyl)-3H-imidazo[4,5-b]pyridin-7-yl]-1-piperidyl]-[4-(trifluoromethoxy)phenyl]methanone;
[4-[2-[(2R)-pyrrolidin-2-yl]-3H-imidazo[4,5-b]pyridin-7-yl]-1-piperidyl]-[4-(trifluoromethoxy)phenyl]methanone;
(rac)-[4-[2-(azetidin-2-yl)-3H-imidazo[4,5-b]pyridin-7-yl]-1-piperidyl]-[4-(trifluoromethoxy)phenyl]methanone;
(rac)-[4-[2-(3-oxa-9-azabicyclo[3.3.1]nonan-7-yl)-3H-imidazo[4,5-b]pyridin-7-yl]-1-piperidyl]-[4-(trifluoromethoxy)phenyl]methanone;
[4-[2-[(2R)-piperazin-2-yl]-3H-imidazo[4,5-b]pyridin-7-yl]-1-piperidyl]-[4-(trifluoromethoxy)phenyl]methanone;
(rac)-[4-[2-(2-azabicyclo[4.1.0]heptan-7-yl)-3H-imidazo[4,5-b]pyridin-7-yl]-1-piperidyl]-[4-(trifluoromethoxy)phenyl]methanone;
(rac)-[4-[2-(3-oxabicyclo[3.1.0]hexan-6-yl)-3H-imidazo[4,5-b]pyridin-7-yl]-1-piperidyl]-[4-(trifluoromethoxy)phenyl]methanone
(rac)-[4-[2-(1,4-oxazepan-2-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(6S)-7-oxa-4-azaspiro[2.5]octan-6-yl]-3H-imidazo[4,5-b]pyridin-7-yl]-1-piperidyl]methanone;
[2-amino-4-(trifluoromethoxy)phenyl]-[4-[2-[(6R)-7-oxa-4-azaspiro[2.5]octan-6-yl]-3H-imidazo[4,5-b]pyridin-7-yl]-1-piperidyl]methanone;
[4-[2-[(6R)-7-oxa-4-azaspiro[2.5]octan-6-yl]-3H-imidazo[4,5-b]pyridin-7-yl]-1-piperidyl]-[4-(trifluoromethoxy)phenyl]methanone;
[4-[2-[(6S)-7-oxa-4-azaspiro[2.5]octan-6-yl]-3H-imidazo[4,5-b]pyridin-7-yl]-1-piperidyl]-[4-(trifluoromethoxy)phenyl]methanone;
(rac)-[4-[2-(azepan-3-yl)-3H-imidazo[4,5-b]pyridin-7-yl]-1-piperidyl]-[4-(trifluoromethoxy)phenyl]methanone
[4-[2-[(3S)-4-amino-1-methyl-2-oxabicyclo[2.1.1]hexan-3-yl]-3H-imidazo[4,5-b]pyridin-7-yl]-1-piperidyl]-[2-amino-4-(trifluoromethoxy)phenyl]methanone;
[4-[2-[(3R)-4-amino-1-methyl-2-oxabicyclo[2.1.1]hexan-3-yl]-3H-imidazo[4,5-b]pyridin-7-yl]-1-piperidyl]-[2-amino-4-(trifluoromethoxy)phenyl]methanone;
(rac)-[4-(2-tetrahydropyran-4-yl-3H-imidazo[4,5-b]pyridin-7-yl)azepan-1-yl]-[4-(trifluoromethoxy)phenyl]methanone;
(rac)-[2-amino-4-(trifluoromethoxy)phenyl]-[4-(2-tetrahydropyran-4-yl-3H-imidazo[4,5-b]pyridin-7-yl)azepan-1-yl]methanone;
(rac)-[4-[2-(3-fluoro-4-piperidyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(3S)-1,1-dioxothiolan-3-yl]-3H-imidazo[4,5-b]pyridin-7-yl]-1-piperidyl]methanone;
[2-amino-4-(trifluoromethoxy)phenyl]-[4-[2-[(3R)-1,1-dioxothiolan-3-yl]-3H-imidazo[4,5-b]pyridin-7-yl]-1-piperidyl]methanone;
[4-[2-[(2R)-1,4-oxazepan-2-yl]-3H-imidazo[4,5-b]pyridin-7-yl]-1-piperidyl]-[4-(trifluoromethoxy)phenyl]methanone;
[4-[2-[(2S)-1,4-oxazepan-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(2R)-1,4-oxazepan-2-yl]-3H-imidazo[4,5-b]pyridin-7-yl]-1-piperidyl]methanone;
[2-amino-4-(trifluoromethoxy)phenyl]-[4-[2-[(2S)-1,4-oxazepan-2-yl]-3H-imidazo[4,5-b]pyridin-7-yl]-1-piperidyl]methanone;
[(4R)-4-(2-tetrahydropyran-4-yl-3H-imidazo[4,5-b]pyridin-7-yl)azepan-1-yl]-[4-(trifluoromethoxy)phenyl]methanone;
[(4S)-4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[(4R)-4-(2-tetrahydropyran-4-yl-3H-imidazo[4,5-b]pyridin-7-yl)azepan-1-yl]methanone;
[2-amino-4-(trifluoromethoxy)phenyl]-[(4S)-4-(2-tetrahydropyran-4-yl-3H-imidazo[4,5-b]pyridin-7-yl)azepan-1-yl]methanone;
[4-[2-[(2R,3S)-3-aminotetrahydrofuran-2-yl]-3H-imidazo[4,5-b]pyridin-7-yl]-1-piperidyl]-[2-amino-4-(trifluoromethoxy)phenyl]methanone;
[4-[2-[(2S,3R)-3-aminotetrahydrofuran-2-yl]-3H-imidazo[4,5-b]pyridin-7-yl]-1-piperidyl]-[2-amino-4-(trifluoromethoxy)phenyl]methanone;
(rac)-[4-[2-(1,1-dioxothiolan-3-yl)-3H-imidazo[4,5-b]pyridin-7-yl]-1-piperidyl]-[4-(trifluoromethoxy)phenyl]methanone;
[4-[2-[(3S)-azepan-3-yl]-3H-imidazo[4,5-b]pyridin-7-yl]-1-piperidyl]-[4-(trifluoromethoxy)phenyl]methanone;
[4-[2-[(3R)-azepan-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[(3S)-azepan-3-yl]-3H-imidazo[4,5-b]pyridin-7-yl]-1-piperidyl]methanone;
[2-amino-4-(trifluoromethoxy)phenyl]-[4-[2-[(3R)-azepan-3-yl]-3H-imidazo[4,5-b]pyridin-7-yl]-1-piperidyl]methanone;
(rac)-[2-amino-4-(pentafluoro-λ⁶-sulfanyl)phenyl]-[4-(2-tetrahydrofuran-2-yl-3H-imidazo[4,5-b]pyridin-7-yl)-1-piperidyl]methanone;
(rac)-[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
[4-(pentafluoro-λ⁶-sulfanyl)phenyl]-[4-[2-[(2S)-tetrahydrofuran-2-yl]-3H-imidazo[4,5-b]pyridin-7-yl]-1-piperidyl]methanone;
[4-(pentafluoro-λ⁶-sulfanyl)phenyl]-[4-[2-[(2R)-tetrahydrofuran-2-yl]-3H-imidazo[4,5-b]pyridin-7-yl]-1-piperidyl]methanone;
[4-(pentafluoro-λ⁶-sulfanyl)phenyl]-[4-[2-[(3R)-tetrahydrofuran-3-yl]-3H-imidazo[4,5-b]pyridin-7-yl]-1-piperidyl]methanone;
[4-(pentafluoro-λ⁶-sulfanyl)phenyl]-[4-[2-[(3S)-tetrahydrofuran-3-yl]-3H-imidazo[4,5-b]pyridin-7-yl]-1-piperidyl]methanone;
[4-[2-[(2R)-morpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[(3R)-tetrahydrofuran-3-yl]-3H-imidazo[4,5-b]pyridin-7-yl]-1-piperidyl]methanone;
[2-amino-4-(pentafluoro-λ⁶-sulfanyl)phenyl]-[4-[2-[(3S)-tetrahydrofuran-3-yl]-3H-imidazo[4,5-b]pyridin-7-yl]-1-piperidyl]methanone;
[4-[2-[(2S)-morpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[(2S)-morpholin-2-yl]-3H-imidazo[4,5-b]pyridin-7-yl]-1-piperidyl]methanone;
[4-[2-[(2S)-4-methylmorpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[(2R)-morpholin-2-yl]-3H-imidazo[4,5-b]pyridin-7-yl]-1-piperidyl]methanone;
[4-[2-[(2R)-4-methylmorpholin-2-yl]-3H-imidazo[4,5-b]pyridin-7-yl]-1-piperidyl]-[4-(pentafluoro-λ⁶-sulfanyl)phenyl]methanone;
[4-(pentafluoro-λ⁶-sulfanyl)phenyl]-[(4S)-4-(2-tetrahydropyran-4-yl-3H-imidazo[4,5-b]pyridin-7-yl)azepan-1-yl]methanone;
[4-(pentafluoro-λ⁶-sulfanyl)phenyl]-[(4R)-4-(2-tetrahydropyran-4-yl-3H-imidazo[4,5-b]pyridin-7-yl)azepan-1-yl]methanone;
[4-[2-[(1R,4R)-2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
[(2R,4R)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[(2S,4S)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[(2R,4S)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[(2S,4R)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-[(1R,4R)-2-oxa-5-azabicyclo[2.2.1]heptan-1-yl]-3H-imidazo[4,5-b]pyridin-7-yl]-1-piperidyl]methanone;
(rac)-[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
(rac)-[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[2-amino-4-(pentafluoro-λ⁶-sulfanyl)phenyl]-[(2S,4S)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(2R,4R)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(2S,4R)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(2R,4S)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(4S)-4-(2-tetrahydropyran-4-yl-3H-imidazo[4,5-b]pyridin-7-yl)azepan-1-yl]methanone;
[2-amino-4-(pentafluoro-λ⁶-sulfanyl)phenyl]-[(4R)-4-(2-tetrahydropyran-4-yl-3H-imidazo[4,5-b]pyridin-7-yl)azepan-1-yl]methanone;
[2-amino-4-(pentafluoro-λ⁶-sulfanyl)phenyl]-[(3R)-3-(2-tetrahydropyran-4-yl-3H-imidazo[4,5-b]pyridin-7-yl)pyrrolidin-1-yl]methanone;
[2-amino-4-(pentafluoro-λ⁶-sulfanyl)phenyl]-[(3S)-3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[(3R)-3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[(3S)-3-(2-tetrahydropyran-4-yl-3H-imidazo[4,5-b]pyridin-7-yl)pyrrolidin-1-yl]methanone;
(rac)-[4-(6-fluoro-2-morpholin-2-yl-3H-imidazo[4,5-b]pyridin-7-yl)-1-piperidyl]-[4-(pentafluoro-λ⁶-sulfanyl)phenyl]methanone;
(rac)-[4-(6-fluoro-2-tetrahydropyran-3-yl-3H-imidazo[4,5-b]pyridin-7-yl)-1-piperidyl]-[4-(pentafluoro-λ⁶-sulfanyl)phenyl]methanone;
(rac)-[2-amino-4-(pentafluoro-λ⁶-sulfanyl)phenyl]-[4-(6-fluoro-2-tetrahydropyran-3-yl-3H-imidazo[4,5-b]pyridin-7-yl)-1-piperidyl]methanone;
(rac)-[4-(6-fluoro-2-tetrahydrofuran-3-yl-3H-imidazo[4,5-b]pyridin-7-yl)-1-piperidyl]-[4-(pentafluoro-λ⁶-sulfanyl)phenyl]methanone;
(rac)-[2-amino-4-(pentafluoro-λ⁶-sulfanyl)phenyl]-[4-[6-fluoro-2-(oxepan-4-yl)-3H-imidazo[4,5-b]pyridin-7-yl]-1-piperidyl]methanone;
(rac)-[4-[6-fluoro-2-(oxepan-4-yl)-3H-imidazo[4,5-b]pyridin-7-yl]-1-piperidyl]-[4-(pentafluoro-λ⁶-sulfanyl)phenyl]methanone;
(rac)-[2-amino-4-(pentafluoro-λ⁶-sulfanyl)phenyl]-[4-(6-fluoro-2-tetrahydrofuran-3-yl-3H-imidazo[4,5-b]pyridin-7-yl)-1-piperidyl]methanone;
[4-[2-[(2R,6R)-6-methylmorpholin-2-yl]-3H-imidazo[4,5-b]pyridin-7-yl]-1-piperidyl]-[4-(pentafluoro-λ⁶-sulfanyl)phenyl]methanone;
(rac)-[2-amino-4-(pentafluoro-λ⁶-sulfanyl)phenyl]-[4-(6-fluoro-2-tetrahydrofuran-2-yl-3H-imidazo[4,5-b]pyridin-7-yl)-1-piperidyl]methanone;
(rac)-[4-(6-fluoro-2-tetrahydrofuran-2-yl-3H-imidazo[4,5-b]pyridin-7-yl)-1-piperidyl]-[4-(pentafluoro-λ⁶-sulfanyl)phenyl]methanone;
(rac)-[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone;
(rac)-[4-(6-fluoro-2-tetrahydropyran-2-yl-3H-imidazo[4,5-b]pyridin-7-yl)-1-piperidyl]-[4-(pentafluoro-λ⁶-sulfanyl)phenyl]methanone;
[4-[6-fluoro-2-[(1R,4R)-2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
[4-[2-[(1R,3R)-3-methoxycyclohexyl]-3H-imidazo[4,5-b]pyridin-7-yl]-1-piperidyl]-[4-(pentafluoro-λ⁶-sulfanyl)phenyl]methanone;
[4-[2-[(1S,3S)-3-methoxycyclohexyl]-3H-imidazo[4,5-b]pyridin-7-yl]-1-piperidyl]-[4-(pentafluoro-λ⁶-sulfanyl)phenyl]methanone;
[4-[2-[(1S,3R)-3-methoxycyclohexyl]-3H-imidazo[4,5-b]pyridin-7-yl]-1-piperidyl]-[4-(pentafluoro-λ⁶-sulfanyl)phenyl]methanone;
[4-[2-[(1R,3S)-(3-methoxycyclohexyl]-3H-imidazo[4,5-b]pyridin-7-yl]-1-piperidyl]-[4-(pentafluoro-λ⁶-sulfanyl)phenyl]methanone;
(rac)-[4-(6-fluoro-2-thiomorpholin-2-yl-3H-imidazo[4,5-b]pyridin-7-yl)-1-piperidyl]-[4-(pentafluoro-λ⁶-sulfanyl)phenyl]methanone;
[4-[6-fluoro-2-[(3S)-tetrahydrofuran-3-yl]-3H-imidazo[4,5-b]pyridin-7-yl]-1-piperidyl]-[4-(pentafluoro-λ⁶-sulfanyl)phenyl]methanone;
[4-[6-fluoro-2-[(3R)-tetrahydrofuran-3-yl]-3H-imidazo[4,5-b]pyridin-7-yl]-1-piperidyl]-[4-(pentafluoro-λ⁶-sulfanyl)phenyl]methanone;
[4-[6-fluoro-2-[(2R,6R)-6-methylmorpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[(3S)-tetrahydrofuran-3-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[(3R)-tetrahydrofuran-3-yl]-3H-imidazo[4,5-b]pyridin-7-yl]-1-piperidyl]methanone;
[4-[6-fluoro-2-[(2R)-morpholin-2-yl]-3H-imidazo[4,5-b]pyridin-7-yl]-1-piperidyl]-[4-(pentafluoro-λ⁶-sulfanyl)phenyl]methanone;
[4-[6-fluoro-2-[(2S)-morpholin-2-yl]-3H-imidazo[4,5-b]pyridin-7-yl]-1-piperidyl]-[4-(pentafluoro-λ⁶-sulfanyl)phenyl]methanone;
(rac)-5-[6-fluoro-7-[1-[4-(pentafluoro-λ⁶-sulfanyl)benzoyl]-4-piperidyl]-3H-imidazo[4,5-b]pyridin-2-yl]piperidin-2-one;
[2-amino-4-(pentafluoro-λ⁶-sulfanyl)phenyl]-[4-[2-[(1R,3S)-3-methoxycyclohexyl]-3H-imidazo[4,5-b]pyridin-7-yl]-1-piperidyl]methanone;
[2-amino-4-(pentafluoro-λ⁶-sulfanyl)phenyl]-[4-[2-[(1S,3R)-3-methoxycyclohexyl]-3H-imidazo[4,5-b]pyridin-7-yl]-1-piperidyl]methanone;
[2-amino-4-(pentafluoro-λ⁶-sulfanyl)phenyl]-[4-[2-[(1R,3R)-3-methoxycyclohexyl]-3H-imidazo[4,5-b]pyridin-7-yl]-1-piperidyl]methanone;
[2-amino-4-(pentafluoro-λ⁶-sulfanyl)phenyl]-[4-[2-[(1S,3S)-3-methoxycyclohexyl]-3H-imidazo[4,5-b]pyridin-7-yl]-1-piperidyl]methanone;
[4-[6-fluoro-2-[(4S)-oxepan-4-yl]-3H-imidazo[4,5-b]pyridin-7-yl]-1-piperidyl]-[4-(pentafluoro-λ⁶-sulfanyl)phenyl]methanone;
[4-[6-fluoro-2-[(4R)-oxepan-4-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[(4S)-oxepan-4-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[(4R)-oxepan-4-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[(2R)-tetrahydrofuran-2-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[(2S)-tetrahydrofuran-2-yl]-3H-imidazo[4,5-b]pyridin-7-yl]-1-piperidyl]methanone;
[4-[6-fluoro-2-[(2R)-tetrahydrofuran-2-yl]-3H-imidazo[4,5-b]pyridin-7-yl]-1-piperidyl]-[4-(pentafluoro-λ⁶-sulfanyl)phenyl]methanone;
[4-[6-fluoro-2-[(2S)-tetrahydrofuran-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[(2R)-tetrahydropyran-2-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[(2S)-tetrahydropyran-2-yl]-3H-imidazo[4,5-b]pyridin-7-yl]-1-piperidyl]methanone;
(rac)-[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;
[4-[6-fluoro-2-[(2R)-tetrahydropyran-2-yl]-3H-imidazo[4,5-b]pyridin-7-yl]-1-piperidyl]-[4-(pentafluoro-λ⁶-sulfanyl)phenyl]methanone;
[4-[6-fluoro-2-[(2S)-tetrahydropyran-2-yl]-3H-imidazo[4,5-b]pyridin-7-yl]-1-piperidyl]-[4-(pentafluoro-λ⁶-sulfanyl)phenyl]methanone;
(rac)-[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone;
(rac)-2-[6-fluoro-7-[1-[4-(pentafluoro-λ⁶-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylic acid;
(rac)-[4-[2-(1,1-dioxo-1,4-thiazinan-2-yl)-6-fluoro-3H-imidazo[4,5-b]pyridin-7-yl]-1-piperidyl]-[4-(pentafluoro-λ⁶-sulfanyl)phenyl]methanone; and
(rac)-7-[6-fluoro-7-[1-[4-(trifluoromethoxy)benzoyl]-4-piperidyl]-3H-imidazo[4,5-b]pyridin-2-yl]-2-oxa-5-azaspiro[3.5]nonane-5-carboxylic acid,
   and the pharmaceutically acceptable salts thereof.

In embodiments, the compound is selected from the compounds produced in Examples 1 to 461 (i.e., from the group consisting of Compounds 1-461), and the pharmaceutically acceptable salts thereof. In other embodiments, the compound is selected from the compounds obtainable by the synthetic methods described in any one of Examples 1 to 461 (i.e., the methods for synthesising Compounds 1-461), and the pharmaceutically acceptable salts thereof. In embodiments, the compound is selected from the compounds produced in Examples 1 to 68 (i.e., from the group consisting of Compounds 1-68), and the pharmaceutically acceptable salts thereof. In other embodiments, the compound is selected from the compounds obtainable by the synthetic methods described in any one of Examples 1 to 68 (i.e., the methods for synthesising Compounds 1-68), and the pharmaceutically acceptable salts thereof.

In embodiments, the compound is selected from the compound of Examples 70, 75, 97, 98, 101, 121, 131, 133, 150, 151, 156, 170, 173, 174, 178, 184, 185, 188, 189, 197, 198, 199, 204, 222, 257, 297, 302, 305, 308, 311, 314, 316, 318, 319, 328, 331, 334, 335, 343, 344, 346, 349, 350, 356, 357, 364, 365, 366, 372, 379, 381, 382, 385, 392, 398, 399, 403, 404, 405, 409, 416, 418, 420, 423, 426, 427, 428, 429, 432, 433, 435, 436, 437, 443, 444, 447, 451, 453, 458, 459, and 460. In embodiments, the compound is selected from the compound of Examples 97, 98, 121, 133, 150, 156, 173, 178, 199, 204, 257, 349, 350, 356, 364, 365, 366, 372, 381, 382, 392, 398, 405, 426, 427, 428, 432, 436, 437, 443, 447, 451, and 460. In embodiments, the compound is selected from the compound of Examples 70, 75, 101, 131, 151, 170, 174, 184, 185, 188, 189, 197, 198, 222, 297, 302, 305, 308, 311, 314, 316, 318, 319, 328, 331, 334, 335, 343, 344, 346, 357, 379, 385, 399, 403, 404, 409, 416, 418, 420, 423, 429, 433, 435, 444, 453, 458, and 459.

In embodiments, the compound of the disclosure is characterised according to its inhibitory activity against ERK5, e.g., as measured according to the cell-based assay or cell-free assay described in the examples below. In embodiments, the compound has an IC₅₀ value of less than about 5 μM against ERK5 (e.g., when measured according to the cell-free assay described below). In embodiments, the compound has an IC₅₀ value of less than about 2 μM, e.g., less than about 1 μM, 0.5 μM, 0.2 μM, 100 nM, or 50 nM against ERK5 (e.g., when measured according to the cell-free assay described below).

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 59, 60, 61, 62, 63, 64, 65, 66, 67, and 68. In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 53, 54, 55, 56, 57, 60, 61, 62, 63, 64, 65, 66, 67, and 68. In other embodiments, the compound is selected from the compound of Examples 1, 2, 3, 4, 5, 6, 7, 8, 9, 11, 12, 13, 15, 16, 17, 19, 20, 21, 22, 23, 24, 25, 26, 27, 28, 29, 30, 31, 32, 33, 34, 36, 37, 38, 39, 42, 43, 44, 45, 46, 47, 50, 51, 53, 54, 56, 57, 61, 62, 63, 65, 67, and 68. In other embodiments, the compound is selected from the compound of Examples 1, 2, 3, 4, 5, 6, 7, 8, 9, 11, 12, 13, 15, 16, 17, 19, 20, 22, 23, 24, 25, 26, 27, 28, 29, 30, 31, 32, 34, 36, 37, 39, 42, 43, 44, 46, 47, 50, 51, 53, 56, 57, 61, 62, 63, 65, 67, and 68. In other embodiments, the compound is selected from the compound of Examples 1, 2, 3, 4, 5, 6, 7, 8, 9, 11, 12, 13, 15, 16, 22, 23, 24, 25, 26, 27, 28, 29, 34, 36, 37, 43, 44, 47, 50, 51, 57, 63, 65, 67, and 68. In other embodiments, the compound is selected from Compounds 3, 4, 5, 6, 7, 8, 9, 11, 12, 13, 15, 16, 22, 29, 37, 44, 51, 57, 63, and 65. In other embodiments, the compound is selected from Compounds 3, 4, 5, 9, 12, 13, 22, 29, 37, 44, and 65.

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 59, 60, 61, 62, 63, 64, 65, 66, 67, 68, 69, 70, 71, 72, 73, 74, 75, 76, 77, 80, 81, 82, 83, 84, 86, 88, 90, 91, 93, 94, 95, 96, 97, 98, 99, 100, 101, 102, 103, 104, 105, 106, 108, 112, 113, 114, 116, 118, 119, 120, 121, 122, 123, 124, 125, 126, 127, 128, 129, 130, 131, 132, 133, 134, 136, 137, 138, 140, 141, 142, 143, 144, 145, 146, 147, 148, 149, 150, 151, 152, 154, 155, 156, 157, 158, 159, 160, 161, 163, 164, 166, 167, 168, 169, 170, 171, 172, 173, 174, 175, 176, 177, 178, 179, 180, 181, 182, 183, 184, 185, 186, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 385, 387, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 446, 447, 448, 449, 450, 451, 452, 453, 454, 455, 456, 457, 458, 459, 460, and 461. In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 53, 54, 55, 56, 57, 60, 61, 62, 63, 64, 65, 66, 67, 68, 69, 70, 71, 72, 73, 74, 75, 76, 77, 81, 82, 83, 90, 91, 93, 94, 95, 96, 97, 98, 100, 101, 102, 103, 104, 108, 119, 121, 122, 123, 124, 125, 126, 127, 128, 129, 130, 131, 132, 133, 134, 136, 137, 138, 140, 141, 142, 143, 144, 145, 146, 147, 148, 149, 150, 151, 152, 154, 155, 156, 157, 158, 159, 160, 161, 163, 164, 166, 167, 168, 169, 170, 171, 172, 173, 174, 175, 176, 177, 178, 179, 180, 181, 182, 183, 184, 185, 188, 189, 191, 192, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 231, 232, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 342, 343, 344, 345, 346, 347, 349, 350, 351, 353, 354, 355, 356, 357, 358, 361, 364, 365, 366, 367, 368, 369, 370, 371, 372, 374, 375, 376, 377, 378, 379, 381, 382, 383, 384, 385, 386, 387, 388, 390, 391, 392, 393, 395, 397, 398, 399, 400, 401, 402, 403, 404, 405, 406, 407, 408, 410, 411, 412, 413, 414, 415, 416, 417, 418, 419, 420, 421, 422, 423, 424, 425, 426, 427, 428, 429, 431, 432, 433, 435, 436, 437, 438, 439, 440, 441, 442, 443, 444, 446, 447, 448, 449, 451, 453, 454, 456, 457, 458, 459, 460, and 461. In other embodiments, the compound is selected from the compound of Examples 1, 2, 3, 4, 5, 6, 7, 8, 9, 11, 12, 13, 15, 16, 17, 19, 20, 21, 22, 23, 24, 25, 26, 27, 28, 29, 30, 31, 32, 33, 34, 36, 37, 38, 39, 42, 43, 44, 45, 46, 47, 50, 51, 53, 54, 56, 57, 61, 62, 63, 65, 67, 68, 69, 70, 71, 72, 73, 74, 75, 76, 77, 81, 82, 90, 91, 93, 94, 95, 96, 97, 98, 100, 101, 102, 103, 108, 119, 121, 122, 123, 124, 125, 126, 127, 128, 129, 130, 131, 132, 133, 137, 138, 140, 142, 143, 144, 145, 146, 148, 149, 150, 151, 152, 154, 155, 156, 157, 159, 160, 161, 163, 164, 166, 167, 168, 169, 170, 173, 174, 175, 176, 177, 178, 179, 180, 181, 182, 183, 184, 185, 188, 189, 191, 192, 194, 195, 196, 197, 198, 199, 200, 202, 203, 204, 205, 208, 209, 210, 212, 213, 215, 216, 219, 220, 222, 224, 225, 226, 227, 228, 229, 231, 234, 235, 237, 238, 239, 240, 241, 242, 243, 244, 245, 246, 247, 248, 249, 250, 251, 252, 253, 254, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 300, 301, 302, 303, 304, 305, 306, 307, 308, 309, 310, 311, 312, 313, 314, 315, 316, 317, 318, 319, 320, 321, 322, 323, 324, 326, 327, 328, 329, 331, 332, 334, 335, 336, 338, 339, 340, 343, 344, 345, 346, 349, 350, 351, 353, 354, 355, 356, 357, 364, 366, 368, 370, 371, 372, 374, 375, 376, 377, 381, 382, 383, 384, 385, 387, 390, 392, 397, 398, 399, 400, 401, 403, 404, 405, 406, 408, 414, 416, 418, 420, 422, 423, 424, 426, 427, 428, 429, 431, 435, 437, 440, 441, 442, 443, 444, 446, 447, 448, 449, 451, 457, 458, 459, 460, and 461. In other embodiments, the compound is selected from the compound of Examples 1, 2, 3, 4, 5, 6, 7, 8, 9, 11, 12, 13, 15, 16, 17, 19, 20, 22, 23, 24, 25, 26, 27, 28, 29, 30, 31, 32, 34, 36, 37, 39, 42, 43, 44, 46, 47, 50, 51, 53, 56, 57, 61, 62, 63, 65, 67, 68, 69, 70, 72, 73, 74, 75, 76, 81, 82, 93, 94, 95, 96, 97, 98, 100, 101, 102, 103, 119, 121, 122, 123, 124, 125, 126, 127, 128, 129, 130, 131, 132, 133, 140, 143, 144, 145, 146, 148, 149, 150, 151, 152, 154, 155, 156, 157, 159, 160, 161, 164, 166, 167, 168, 169, 170, 173, 174, 175, 176, 177, 178, 179, 180, 181, 182, 183, 184, 185, 188, 189, 191, 194, 195, 196, 197, 198, 199, 200, 202, 203, 204, 205, 208, 210, 213, 216, 219, 220, 222, 224, 225, 227, 229, 231, 234, 235, 237, 238, 239, 241, 244, 245, 246, 247, 248, 249, 250, 251, 252, 253, 254, 256, 257, 258, 260, 261, 262, 264, 265, 266, 267, 268, 269, 270, 271, 272, 274, 275, 276, 277, 278, 279, 280, 281, 282, 283, 284, 285, 286, 287, 288, 289, 290, 291, 292, 294, 295, 296, 297, 298, 300, 301, 302, 303, 304, 305, 306, 307, 308, 309, 311, 312, 313, 314, 315, 316, 318, 319, 320, 322, 323, 324, 326, 327, 328, 329, 331, 332, 334, 335, 338, 340, 343, 344, 345, 346, 349, 350, 351, 354, 356, 357, 364, 370, 372, 374, 375, 377, 381, 382, 383, 384, 385, 387, 390, 392, 397, 398, 405, 406, 408, 418, 420, 422, 423, 424, 426, 427, 428, 429, 435, 440, 441, 443, 444, 446, 447, 448, 449, 451, 457, 458, 459, 460, and 461. In other embodiments, the compound is selected from the compound of Examples 1, 2, 3, 4, 5, 6, 7, 8, 9, 11, 12, 13, 15, 16, 22, 23, 24, 25, 26, 27, 28, 29, 34, 36, 37, 43, 44, 47, 50, 51, 57, 63, 65, 67, 68, 70, 72, 73, 74, 75, 76, 93, 94, 96, 97, 98, 100, 101, 102, 103, 119, 121, 123, 124, 125, 126, 127, 128, 130, 131, 132, 133, 140, 144, 145, 146, 148, 149, 150, 151, 152, 154, 155, 156, 159, 160, 161, 166, 167, 169, 170, 173, 174, 175, 178, 179, 181, 182, 183, 184, 185, 188, 189, 191, 195, 196, 197, 198, 199, 200, 202, 204, 205, 208, 219, 220, 222, 227, 231, 237, 238, 239, 244, 245, 247, 248, 249, 251, 252, 253, 256, 257, 258, 261, 262, 264, 265, 266, 267, 268, 269, 270, 271, 274, 275, 276, 277, 280, 281, 282, 283, 284, 285, 286, 287, 288, 289, 292, 294, 296, 297, 298, 302, 303, 305, 306, 307, 308, 309, 311, 312, 313, 314, 316, 318, 319, 320, 326, 327, 328, 329, 331, 334, 335, 343, 344, 345, 346, 349, 350, 357, 364, 385, 390, 392, 398, 405, 422, 423, 424, 426, 427, 428, 429, 435, 440, 443, 444, 447, 448, 451, 458, and 460. In other embodiments, the compound is selected from the compound of Examples 3, 4, 5, 6, 7, 8, 9, 11, 12, 13, 15, 16, 22, 29, 37, 44, 51, 57, 63, 65, 70, 72, 73, 74, 75, 94, 96, 97, 98, 100, 101, 121, 123, 125, 127, 128, 131, 133, 140, 146, 148, 149, 150, 151, 152, 154, 155, 156, 159, 169, 170, 173, 174, 175, 178, 181, 184, 185, 188, 189, 191, 195, 197, 198, 200, 202, 204, 208, 219, 220, 222, 227, 231, 237, 238, 239, 244, 247, 248, 249, 251, 252, 253, 256, 258, 262, 264, 265, 266, 268, 269, 270, 271, 277, 280, 281, 282, 283, 284, 285, 287, 288, 289, 297, 302, 305, 307, 308, 311, 314, 316, 318, 319, 320, 328, 329, 331, 334, 335, 343, 346, 349, 350, 385, 392, 398, 405, 426, 428, 429, 435, 443, 447, 451, and 460. In other embodiments, the compound is selected from the compound of Examples 3, 4, 5, 9, 12, 13, 22, 29, 37, 44, 65, 72, 97, 101, 123, 127, 128, 133, 140, 146, 149, 150, 151, 152, 154, 156, 169, 170, 173, 174, 175, 178, 184, 185, 188, 189, 191, 197, 202, 204, 208, 219, 222, 237, 249, 251, 253, 256, 262, 268, 270, 271, 281, 282, 283, 284, 285, 287, 288, 289, 307, 318, 343, 428, 435, and 460.

In embodiments, the compound is selected from the compound of Examples 1, 2, 3, 4, 5, 6, 7, 8, 9, 10, 11, 12, 13, 14, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, and 68. In embodiments, the compound is selected from the compound of Examples 1, 2, 3, 4, 5, 6, 7, 8, 9, 10, 11, 12, 13, 14, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 60, 61, 62, 63, 64, 65, 66, 67, and 68. In other embodiments, the compound is selected from the compound of Examples 1, 2, 3, 4, 5, 6, 7, 8, 9, 11, 12, 13, 18, 19, 20, 22, 23, 24, 25, 26, 27, 28, 29, 30, 31, 32, 33, 34, 36, 37, 38, 39, 40, 41, 42, 43, 44, 45, 46, 47, 50, 51, 54, 56, 57, 60, 61, 62, 63, 64, 65, 66, 67, and 68. In other embodiments, the compound is selected from the compound of Examples 1, 2, 3, 4, 5, 6, 7, 8, 9, 11, 12, 13, 19, 20, 22, 23, 24, 25, 26, 27, 28, 29, 30, 31, 32, 33, 34, 36, 37, 38, 39, 42, 43, 44, 45, 46, 47, 50, 51, 56, 57, 60, 61, 63, 65, 67, and 68. In other embodiments, the compound is selected from the compound of Examples 1, 2, 3, 4, 5, 7, 8, 9, 11, 12, 13, 19, 20, 22, 24, 25, 26, 27, 28, 29, 34, 36, 37, 42, 43, 44, 46, 47, 50, 51, 57, 65, 67, and 68. In embodiments, the compound is selected from the compound of Examples 1, 3, 4, 5, 7, 8, 9, 11, 12, 13, 22, 26, 27, 28, 29, 34, 37, 44, 57, and 65. In other embodiments, the compound is selected from the compound of Examples 3, 4, 5, 9, 11, 12, 22, 29, and 37.

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 400, 432, and 436. In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 54, 55, 56, 57, 60, 61, 62, 63, 64, 65, 66, 67, 68, 69, 70, 71, 72, 73, 74, 75, 76, 77, 78, 81, 82, 83, 89, 90, 91, 92, 93, 94, 95, 96, 97, 98, 99, 100, 101, 102, 103, 104, 108, 114, 119, 121, 122, 123, 124, 125, 126, 127, 128, 129, 130, 131, 132, 133, 134, 136, 137, 138, 139, 140, 141, 142, 143, 144, 145, 146, 147, 148, 149, 150, 151, 152, 153, 154, 155, 156, 157, 158, 159, 160, 161, 162, 163, 164, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 432, 433, 434, 435, 436, 437, 438, 439, 440, 441, 442, 443, 444, 447, 451, 452, 453, 454, 456, 457, 458, 459, and 460. In other embodiments, the compound is selected from the compound of Examples 1, 2, 3, 4, 5, 6, 7, 8, 9, 11, 12, 13, 15, 16, 17, 18, 19, 20, 22, 23, 24, 25, 26, 27, 28, 29, 30, 31, 32, 33, 34, 36, 37, 38, 39, 40, 41, 42, 43, 44, 45, 46, 47, 50, 51, 54, 56, 57, 60, 61, 62, 63, 64, 65, 66, 67, 68, 69, 70, 71, 72, 73, 74, 75, 76, 77, 81, 82, 90, 91, 94, 95, 96, 97, 98, 100, 101, 102, 103, 119, 121, 122, 123, 124, 125, 126, 127, 128, 129, 130, 131, 132, 133, 134, 136, 137, 138, 140, 142, 143, 144, 145, 146, 148, 149, 150, 151, 152, 153, 154, 155, 156, 157, 158, 159, 160, 161, 162, 163, 164, 166, 167, 168, 169, 170, 172, 173, 174, 175, 176, 177, 178, 179, 180, 181, 182, 183, 184, 185, 186, 187, 188, 189, 190, 191, 192, 194, 195, 196, 197, 198, 199, 200, 201, 202, 203, 204, 205, 206, 208, 210, 211, 212, 213, 214, 215, 216, 217, 219, 220, 221, 222, 224, 225, 227, 228, 229, 231, 232, 233, 234, 235, 237, 238, 239, 240, 241, 242, 243, 244, 245, 246, 247, 248, 249, 250, 251, 252, 253, 254, 256, 257, 258, 259, 260, 261, 262, 265, 266, 267, 268, 269, 270, 271, 272, 273, 274, 275, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 326, 327, 328, 329, 330, 331, 332, 333, 334, 335, 336, 337, 338, 339, 340, 342, 343, 344, 345, 346, 347, 349, 350, 351, 353, 354, 355, 356, 357, 358, 359, 361, 364, 365, 366, 367, 368, 369, 370, 371, 372, 373, 374, 375, 376, 377, 379, 380, 381, 382, 384, 385, 386, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 432, 433, 434, 435, 436, 437, 438, 439, 440, 441, 442, 443, 444, 447, 451, 453, 454, 456, 457, 458, 459, and 460. In other embodiments, the compound is selected from the compound of Examples 1, 2, 3, 4, 5, 6, 7, 8, 9, 11, 12, 13, 15, 16, 17, 19, 20, 22, 23, 24, 25, 26, 27, 28, 29, 30, 31, 32, 33, 34, 36, 37, 38, 39, 42, 43, 44, 45, 46, 47, 50, 51, 56, 57, 60, 61, 63, 65, 67, 68, 69, 70, 71, 72, 73, 74, 75, 76, 77, 81, 82, 90, 91, 94, 95, 96, 97, 98, 100, 101, 102, 103, 119, 121, 122, 123, 125, 126, 127, 128, 129, 130, 131, 132, 133, 136, 137, 138, 140, 142, 143, 144, 145, 146, 148, 149, 150, 151, 152, 153, 154, 155, 156, 157, 159, 160, 161, 162, 164, 166, 167, 168, 169, 170, 173, 174, 175, 176, 178, 179, 180, 181, 182, 183, 184, 185, 186, 187, 188, 189, 191, 194, 195, 196, 197, 198, 199, 200, 202, 203, 204, 205, 208, 210, 212, 213, 215, 216, 219, 222, 225, 227, 228, 231, 234, 235, 237, 238, 239, 240, 241, 242, 243, 244, 245, 246, 247, 248, 249, 250, 251, 252, 253, 254, 256, 257, 258, 259, 260, 261, 262, 265, 266, 268, 269, 270, 271, 272, 273, 274, 275, 277, 280, 281, 282, 283, 284, 285, 286, 287, 288, 289, 290, 291, 292, 293, 294, 295, 296, 297, 298, 300, 301, 302, 304, 305, 306, 307, 308, 309, 310, 311, 312, 313, 314, 315, 316, 317, 318, 319, 320, 321, 322, 323, 324, 327, 328, 329, 330, 331, 332, 333, 334, 335, 336, 338, 339, 340, 342, 343, 344, 345, 346, 349, 350, 351, 353, 354, 355, 356, 357, 358, 364, 365, 366, 367, 368, 369, 370, 371, 372, 373, 374, 375, 376, 377, 379, 380, 381, 382, 384, 385, 386, 388, 389, 390, 391, 392, 393, 394, 395, 397, 398, 399, 400, 401, 403, 404, 405, 406, 407, 408, 409, 410, 411, 412, 413, 414, 415, 416, 417, 418, 419, 420, 421, 422, 423, 424, 425, 426, 427, 428, 429, 432, 433, 434, 435, 436, 437, 438, 439, 440, 441, 442, 443, 444, 447, 451, 453, 454, 457, 458, 459, and 460. In other embodiments, the compound is selected from the compound of Examples 1, 2, 3, 4, 5, 7, 8, 9, 11, 12, 13, 15, 17, 19, 20, 22, 24, 25, 26, 27, 28, 29, 34, 36, 37, 42, 43, 44, 46, 47, 50, 51, 57, 65, 67, 68, 69, 70, 72, 75, 76, 77, 94, 96, 97, 98, 100, 101, 102, 103, 119, 121, 122, 125, 126, 127, 128, 130, 131, 132, 133, 140, 143, 144, 145, 146, 148, 149, 150, 153, 154, 155, 156, 159, 160, 161, 166, 167, 169, 170, 173, 174, 175, 178, 179, 181, 184, 185, 186, 187, 188, 189, 191, 195, 196, 197, 198, 199, 200, 202, 204, 208, 212, 216, 219, 222, 228, 231, 234, 237, 238, 239, 241, 242, 244, 245, 248, 249, 251, 252, 253, 256, 257, 258, 262, 265, 268, 269, 270, 271, 281, 282, 283, 284, 285, 287, 288, 289, 290, 291, 292, 294, 296, 297, 298, 302, 304, 305, 306, 308, 309, 311, 312, 313, 316, 318, 320, 323, 327, 328, 331, 332, 333, 334, 335, 336, 340, 343, 344, 349, 350, 351, 353, 354, 355, 356, 357, 364, 365, 366, 367, 370, 371, 372, 374, 375, 376, 377, 379, 381, 382, 384, 385, 386, 390, 391, 392, 397, 398, 399, 400, 401, 403, 404, 405, 409, 414, 415, 416, 417, 418, 419, 420, 422, 423, 425, 426, 427, 428, 429, 432, 433, 434, 435, 436, 437, 438, 439, 440, 441, 442, 443, 444, 447, 451, 453, 454, 457, 458, 459, and 460. In embodiments, the compound is selected from the compound of Examples 1, 3, 4, 5, 7, 8, 9, 11, 12, 13, 15, 17, 22, 26, 27, 28, 29, 34, 37, 44, 57, 65, 70, 72, 75, 76, 96, 97, 98, 101, 102, 121, 122, 125, 126, 127, 128, 131, 132, 133, 140, 144, 146, 149, 150, 154, 155, 156, 166, 167, 170, 173, 174, 178, 184, 185, 186, 187, 189, 195, 197, 198, 199, 200, 204, 212, 219, 222, 231, 237, 238, 244, 245, 248, 249, 251, 253, 256, 262, 268, 270, 271, 282, 283, 284, 285, 289, 290, 294, 297, 302, 305, 308, 311, 344, 349, 350, 351, 356, 357, 364, 365, 366, 367, 370, 371, 372, 375, 377, 381, 382, 384, 385, 398, 399, 403, 404, 405, 409, 416, 417, 418, 420, 422, 423, 426, 427, 428, 429, 432, 433, 435, 436, 440, 441, 443, 444, 447, 451, 453, 458, 459, and 460. In other embodiments, the compound is selected from the compound of Examples 3, 4, 5, 9, 11, 12, 15, 17, 22, 29, 37, 75, 97, 101, 121, 125, 127, 128, 131, 133, 146, 150, 154, 156, 166, 170, 173, 174, 178, 186, 187, 189, 197, 198, 199, 200, 204, 222, 237, 251, 253, 256, 270, 283, 284, 305, 350, 356, 357, 365, 366, 372, 381, 382, 385, 398, 405, 418, 426, 427, 428, 429, 432, 435, 436, 443, 447, 451, and 460.

### Pharmaceutical Compositions

The present disclosure provides a pharmaceutical composition comprising a compound described herein (e.g., a compound of Formula (I) or a pharmaceutically acceptable salt thereof), and at least one pharmaceutically acceptable excipient or carrier.

In embodiments, the pharmaceutical composition comprises a compound of Formula (I) or a pharmaceutically acceptable salt thereof. In embodiments, the pharmaceutical composition comprises a compound of Formula (I-A) or a pharmaceutically acceptable salt thereof. In embodiments, the pharmaceutical composition comprises a compound of Formula (I-B) or a pharmaceutically acceptable salt thereof. In embodiments, the pharmaceutical composition comprises a compound of Formula (I-C) or a pharmaceutically acceptable salt thereof. In embodiments, the pharmaceutical composition comprises a compound of Formula (I-D) or a pharmaceutically acceptable salt thereof. In embodiments, the pharmaceutical composition comprises a compound of Formula (II) or a pharmaceutically acceptable salt thereof. In embodiments, the pharmaceutical composition comprises a compound of Formula (III) or a pharmaceutically acceptable salt thereof. In embodiments, the pharmaceutical composition comprises a compound of Formula (IV) or a pharmaceutically acceptable salt thereof. In embodiments, the pharmaceutical composition comprises a compound of Formula (V) or a pharmaceutically acceptable salt thereof. In embodiments, the pharmaceutical composition comprises a compound of Formula (VI) or a pharmaceutically acceptable salt thereof. In embodiments, the pharmaceutical composition comprises a compound of Formula (VI-A) or a pharmaceutically acceptable salt thereof. In embodiments, the pharmaceutical composition comprises a compound of Formula (VII) or a pharmaceutically acceptable salt thereof. In embodiments, the pharmaceutical composition comprises a compound of Formula (VII-A) or a pharmaceutically acceptable salt thereof. In embodiments, the pharmaceutical composition comprises a compound of Formula (VIII) or a pharmaceutically acceptable salt thereof. In embodiments, the pharmaceutical composition comprises a compound of Formula (VIII-A) or a pharmaceutically acceptable salt thereof. In embodiments, the pharmaceutical composition comprises a compound of Formula (IX) or a pharmaceutically acceptable salt thereof. In embodiments, the pharmaceutical composition comprises a compound of Formula (IX-A) or a pharmaceutically acceptable salt thereof. In embodiments, the pharmaceutical composition comprises a compound of Formula (X) or a pharmaceutically acceptable salt thereof. In embodiments, the pharmaceutical composition comprises a compound of Formula (X-A) or a pharmaceutically acceptable salt thereof.

The pharmaceutical compositions of the disclosure may be formulated for administration in solid or liquid form, e.g., using conventional carriers or excipients. Compositions may be adapted for, e.g., oral administration (e.g., as a solution, suspension, tablet, or capsule), parenteral administration (e.g., as a solution, dispersion, suspension, or emulsion, or as a dry powder for reconstitution), or topical application (e.g., as a cream, ointment, patch, or spray to be applied to the skin) using techniques known in the art.

### Medical Uses

Compounds of the present disclosure act as inhibitors of ERK5, which gives them utility in the treatment of ERK5-associated disorders and conditions. In particular, compounds of the disclosure are useful in the treatment of cancers.

Viewed from this aspect, the disclosure provides a method of treatment comprising administering to a subject in need thereof a therapeutically effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament. In a further related aspect, the disclosure provides a compound of the disclosure (e.g., a compound of Formula (I) or a pharmaceutically acceptable salt thereof) for use in therapy.

Compounds of the present disclosure are useful for treating or preventing: diseases or deleterious conditions in which ERK5, or a variant or mutant thereof, is known to play a role; diseases or disorders associated with increased MAPK7 (i.e., ERK5 gene) expression and/or increased ERK5 activity; and diseases or disorders in which inhibition or antagonism of ERK5 activity is beneficial.

In one aspect, the present disclosure provides a method of treating or preventing a disease or disorder mediated by ERK5, or a disease or disorder in which ERK5 is implicated, in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of a disease or disorder mediated by ERK5, or a disease or disorder in which ERK5 is implicated. In a further related aspect, the disclosure provides a compound of the disclosure (e.g., a compound of Formula (I) or a pharmaceutically acceptable salt thereof) for the treatment or prevention of a disease or disorder mediated by ERK5, or a disease or disorder in which ERK5 is implicated.

In another aspect, the present disclosure provides a method of treating or preventing a disease or disorder associated with ERK5 (e.g., cancer) in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of a disease or disorder associated with ERK5 (e.g., cancer). In a further related aspect, the disclosure provides a compound of the disclosure (e.g., a compound of Formula (I) or a pharmaceutically acceptable salt thereof) for the treatment or prevention of a disease or disorder associated with ERK5 (e.g., cancer).

In another aspect, the present disclosure provides a method of treating or preventing cancer in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of cancer. In a further related aspect, the disclosure provides a compound of the disclosure (e.g., a compound of Formula (I) or a pharmaceutically acceptable salt thereof) for the treatment or prevention of cancer.

In embodiments, the compound reduces angiogenesis, reduces or prevents metastasis, reduces inflammation, blocks tumorigenesis (e.g., in part or completely), reduces evasion of growth suppression, reduces or inhibits growth of cancerous or pre-cancerous cells, supresses proliferation of cancerous or pre-cancerous cells, and/or reduces the survival of cancerous or pre-cancerous cells.

In embodiments, the cancer is characterized by increased MAPK7 (i.e., ERK5 gene) expression and/or increased ERK5 activity. In embodiments, the cancer has elevated ERK5 activity. In embodiments, the cancer overexpresses ERK5. In embodiments, the cancer is characterised by MAPK7 genomic amplification and/or constitutively active ERK5 signalling.

In embodiments, the cancer has genomically amplified ERK5. In embodiments, the cancer has constitutively active ERK5 signalling.

In embodiments, the cancer is a solid tumour (e.g., a melanoma, carcinoma, or blastoma). In other embodiments, the cancer is leukaemia (e.g., chronic lymphocytic leukaemia, CLL; acute myelogenous leukaemia, AML; or chronic myelogenous leukaemia, CML).

In embodiments, the cancer is a primary tumour. In other embodiments, the cancer is a secondary tumour (e.g., a metastatic tumour).

In embodiments, the cancer is selected from breast cancer (e.g., ductal breast carcinoma, or breast adenocarcinoma), liver cancer, kidney cancer (e.g., hepatocellular carcinoma), prostate cancer, colorectal cancer (CRC), lung cancer (e.g., non-small cell lung cancer, NSCLC; lung adenocarcinoma; or lung squamous cell carcinoma), pancreatic cancer (e.g., adenocarcinoma), ovarian cancer, brain cancer (e.g., glioblastoma), cervical cancer (e.g., adenocarcinoma), gastric cancer, skin cancer, bile duct cancer (e.g., cholangiocarcinoma), nervous system cancer (e.g., neuroblastoma), and melanoma.

In embodiments, the cancer is selected from leukaemia (e.g., acute leukaemia, acute lymphocytic leukaemia, acute myelocytic leukaemia, acute myeloblastic leukaemia, acute promyelocytic leukaemia, acute myelomonocytic leukaemia, acute monocytic leukaemia, acute erythroleukemia, chronic leukaemia, chronic myelocytic leukaemia, or chronic lymphocytic leukaemia), polycythaemia vera, lymphoma (e.g., Hodgkin's disease or non-Hodgkin's disease), Waldenström macroglobulinemia, and multiple myeloma.

In embodiments, the cancer is selected from leukaemia (e.g., chronic myeloid leukaemia), breast cancer, multiple myeloma, colon cancer, renal cell carcinoma, mesothelioma, adenocarcinoma, neuroblastoma, and hepatocellular carcinoma.

In another aspect, the disclosure provides a method of inhibiting ERK5 activity, the method comprising contacting ERK5 (e.g., a cell comprising ERK5) with a compound of the present disclosure (e.g., a compound of Formula (I) or a pharmaceutically acceptable salt thereof). In embodiments, the method is an in vitro or ex vivo method. In other embodiments the method is an in vivo method. In a related aspect, the disclosure provides an in vitro method of inhibiting ERK5 activity in a cell, the method comprising contacting the cell with a compound of the present disclosure (e.g., a compound of Formula (I) or a pharmaceutically acceptable salt thereof).

Compounds of the present disclosure (e.g., compounds of Formula (I)) and the pharmaceutically acceptable salts thereof may be administered as pharmaceutical compositions, which may optionally comprise one or more pharmaceutically acceptable excipients.

It will be appreciated that the methods and treatments of the various aspects of this disclosure may be effected by administering to a subject an effective amount of a compound of the disclosure (e.g., a compound of Formula (I) or a pharmaceutically acceptable salt thereof), in the form of a pharmaceutical composition, which may optionally comprise one or more pharmaceutically acceptable excipients, as described herein.

The compounds of the disclosure may be used alone (e.g., as a monotherapy) or in combination with one or more cancer therapies.

Having been generally described herein, the follow non-limiting examples are provided to further illustrate this disclosure.

### EXAMPLES

### General Synthetic Schemes

The following scheme, Scheme 1A, illustrates an exemplary way of preparing compounds in accordance with the present disclosure and examples:

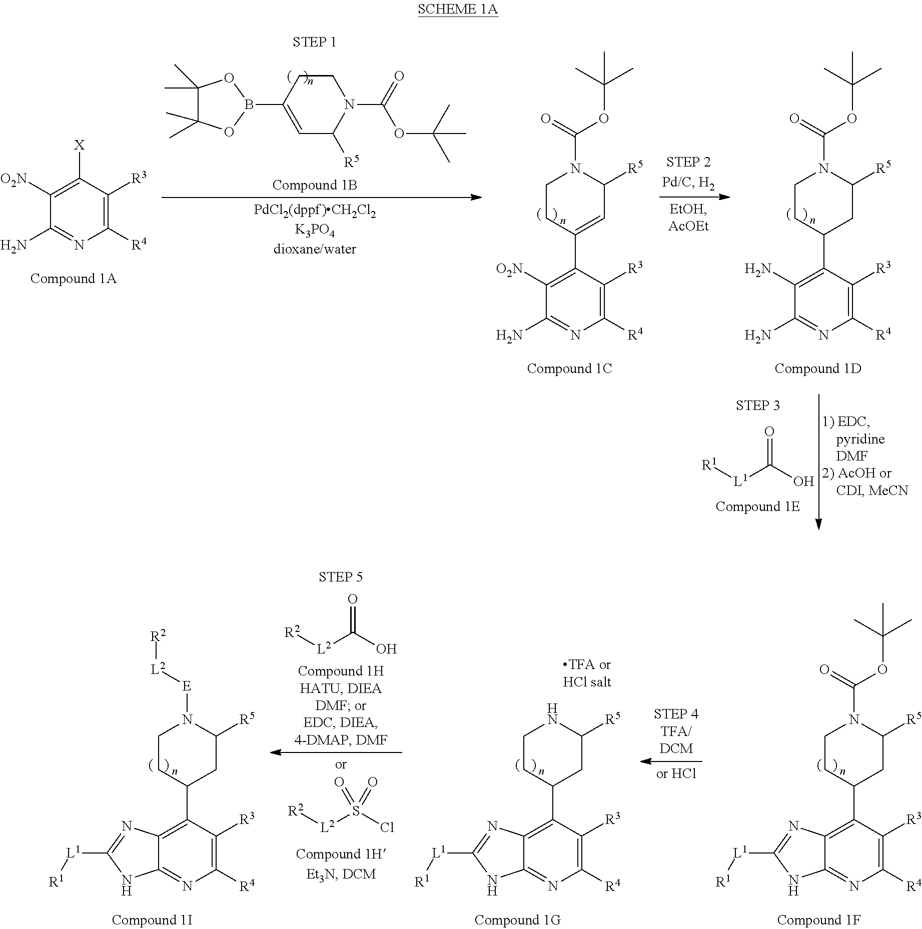

According to Scheme 1A (in which R¹, L¹, R², L², R³, R⁴, R³, and n may be, e.g., as described above), Compound 1C can be obtained in STEP 1 by Suzuki coupling between Compound 1A (wherein X may be I, Br, or Cl) and Compound 1B using, for example, a catalyst such as [1,1′-bis(diphenylphosphino)ferrocene]dichloropalladium(II) complexed with DCM in a mixture of dioxane and water and in the presence of a base, such as potassium carbonate, by heating to reflux of the solvent. Compound 1C can be reduced in STEP 2 to Compound 1D by hydrogenation with a catalyst such as Pd/C under hydrogen pressure (H₂) around 5 bars at 40° C., for example. Compound 1D can then be converted to Compound 1F in STEP 3 by adding it to a premixed solution of a carboxylic acid (Compound 1E) and CDI in acetonitrile followed by heating to reflux, for example. Alternatively, Compound 1D can be coupled with Compound 1E using conditions known by the person skilled in the art, such as EDC in a solvent like DMF in presence of a base such as pyridine followed by heating with AcOH up to 80° C. to provide compound 1F. Compound 1F can be converted in STEP 4 to Compound 1G by treatment with an acid such as neat TFA or a solution of HCl in dioxane in a solvent such as MeOH. In STEP 5, Compound 1I in which E is —C(O)— can be obtained by peptide coupling between Compound 1G and Compound 1H using conditions known by the person skilled in the art, such as EDC in a solvent like DMF in presence of 4-DMAP and DIEA or HATU in a solvent like DMF in presence of DIEA. Compound 1I in which E is —S(O)₂— can be obtained by treatment of Compound 1G with Compound 1H′ using conditions known by the person skilled in the art, e.g., treatment with a base such as triethylamine in a solvent like DCM.

An alternative way to convert Compound 1D into Compound 1I is illustrated in the following scheme, Scheme 1B:

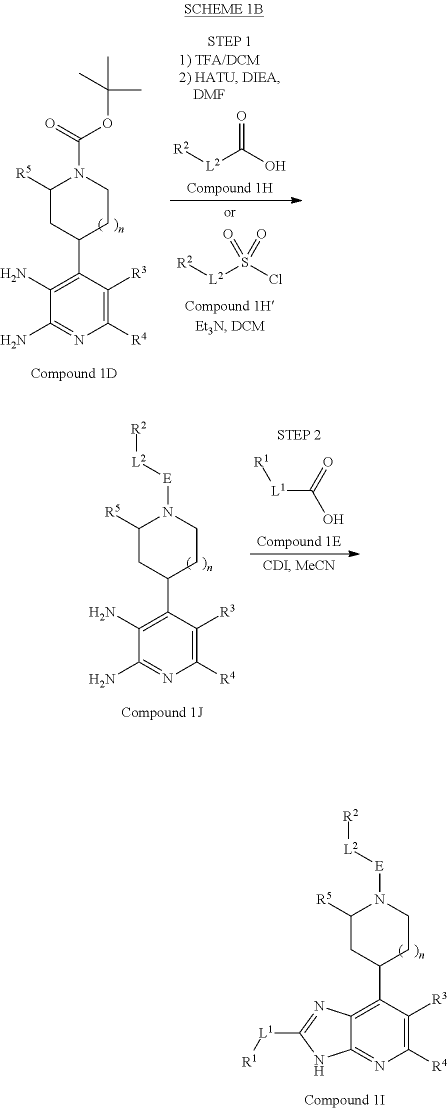

According to Scheme 1B (in which R¹, L¹, R², L², R³, R⁴, R⁵, and n may be as described above), Compound 1J can be obtained in STEP 1 by treatment of Compound 1D with an acid such as TFA in a solvent such as DCM followed by either peptide coupling with Compound 1H using conditions known by the person skilled in the art, such as HATU in a solvent like DMF in presence of a base such as DIEA, or by reacting with Compound 1H′ and a base such as triethylamine in a solvent like DCM for instance. Compound 1J can then be converted to Compound 1I (in which E is —C(O)—, or —S(O)₂—) in STEP 2 by coupling with Compound IE using conditions such as, for example, CDI in acetonitrile followed by heating to reflux.

Compounds in which E is —O— may be synthesized by analogy to the methods shown above, e.g., using a modified version of Scheme 1A. For example, an analogue of Compound IG having a carbon atom in place of the piperidine nitrogen and carrying a protected hydroxyl group (Compound 1G′ below) may be deprotected and coupled to the R²-containing group by alkylation (e.g., using a Williamson reaction to yield an alkyl ether) or a Mitsunobu reaction (e.g., to yield an aryl or heteroaryl ether). This is illustrated in Scheme 1C below:

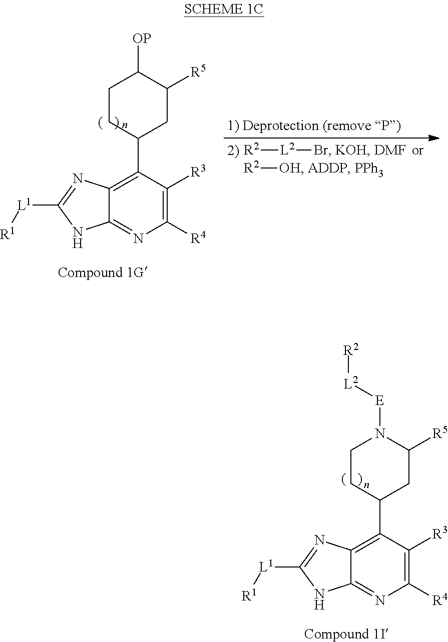

### Experimental Techniques

¹H NMR Spectra at 400 and 500 MHz were performed on a Bruker Avance DRX-400 and Bruker Avance DPX-500 spectrometer, respectively, with the chemical shifts (6 in ppm) in the solvent dimethyl sulfoxide-d₆ (DMSO-d₆) referenced at 2.5 ppm at the quoted temperatures. Coupling constants (J) are given in Hertz.

For intermediates in the following synthetic procedures, liquid chromatography/mass spectra (LC/MS) were obtained on a UPLC Acquity Waters instrument, light scattering detector Sedere and SQD Waters mass spectrometer using UV detection DAD 210<l<400 nm and column Acquity UPLC CSH C18 1.7 μm, dimension 2.1×30 mm, mobile phase H₂O+0.0% HCO₂H/CH₃CN+0.0% HCO₂H.

For the compounds prepared as the final products of the synthetic methods below (i.e., for the compounds of Examples 1-461), LC/MS were obtained as specified by Method 1, Method 2, or Method 3.

### Method11

Liquid chromatography/mass spectra (LC/MS) were obtained on a Waters ACQUITY UPLC instrument with Waters SQD2, using positive and negative electrospray ionisation (ES+/−) under the following conditions:

| | |
|---|---|
| Column | ACQUITY CORTECS C18+, 1.7 μm, 2.1 × 50 mm |
| Solvents | A: H₂O + 0.1% formic acid |
| | B: CH₃CN + 0.1% formic acid |
| Column | 40° C. |
| Temperature | |
| Flow Rate | 1 mL/min |
| Gradient | Linear gradient (3 min): From 0 to 2 min: 2 to 100% B; |
| | from 2 to 2.6 min: 100% B; from 2.6 to 2.7 min: 100 to |
| | 2% B; from 2.7 to 3 min: 2% B |
| UV Detection | PDA: 191-499 nm or 210-400 nm |
| MS Detection | SCAN: 85-1500 uma |

### Method 2

Liquid chromatography/mass spectra (LC/MS) were obtained on a Waters ACQUITY UPLC instrument with Waters SQD2, using positive and negative electrospray ionisation (ES+/−) under the following conditions:

| | |
|---|---|
| Column | ACQUITY CSH C18+, 1.7 μm, 2.1 × 50 mm |
| Solvents | A: H₂O + 0.1% formic acid |
| | B: CH₃CN + 0.1% formic acid |
| Column | 60° C. |
| Temperature | |
| Flow Rate | 1 mL/min |
| Gradient | Linear gradient (2.5 min): From 0 to 2.1 min: 3 to 100% |
| | B; from 2.1 to 2.45 min: 100% B; from 2.45 to 2.5 min: |
| | 100 to 3% B; or |
| | Linear gradient (3 min): From 0 to 0.1 min, 3% B; from |
| | 0.1 to 2.1 min: 3 to 97% B; from 2.1 to 2.45 min: 97% |
| | B; from 2.5 to 3 min: 97% B |
| UV Detection | PDA: 195-380 nm |
| MS Detection | SCAN: 85-1500 uma |

### Method 3

Liquid chromatography/mass spectra (LC/MS) were obtained on a Waters ACQUITY UPLC instrument with Waters SQD2, using positive and negative electrospray ionisation (ES+/−) under the following conditions:

| | |
|---|---|
| Column | ACQUITY CSH C18+, 1.7 μm, 2.1 × 50 mm |
| Solvents | A: H₂O + 0.1% formic acid |
| | B: CH₃CN + 0.1% formic acid |
| Column | 60° C. |
| Temperature | |
| Flow Rate | 1 mL/min |
| Gradient | Linear gradient (10 min): From 0 to 8.6 min: 3 to 97% of |
| | B; from 8.6 to 9.6 min: 97% of B; from 9.6 to 9.8 min: |
| | 97 to 3% of B; from 9.8 to 10 min: 3% of B |
| UV Detection | PDA: 195-380 nm |
| MS Detection | SCAN: 85-1500 uma |

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich).

Isomeric separation (e.g., chiral separation) of compounds was performed by one of the following methods as specified in the synthetic methods below.

### Method CS1

Separation of the corresponding mixture (e.g. 0.19 mmol thereof) was performed using a chiralpak IC column (20 μm, 100×230 mm), eluting with (n-Heptane 30% EtOH 70%)+0.1% TEA (flow rate 400 mL/min, UV detection at 265 nm).

### Method CS2

Separation of the corresponding mixture (e.g. 0.30 mmol thereof) was performed using a chiralpak AD column (20 μm, 350×76.5 mm), eluting with (n-Heptane 80% EtOH 20%)+0.10% TEA (flow rate 400 mL/min, UV detection at 265 nm).

### Method CS3

Separation of the corresponding mixture (e.g. 0.08 mmol thereof) was performed using I Cellulose 5 column (5 μm, 250×30 mm), eluting with (n-Heptane 40% EtOH 60%)+0.1% TEA (flow rate 1 mL/min, UV detection at 265 nm).

### Method CS4

Separation of the corresponding mixture (e.g. 0.1 mmol thereof) was performed using a Chiralcel OD-H column (5 μm, 250×30 mm), eluting with (n-Heptane 85% EtOH 15%)+0.10% TEA (flow rate 40 mL/min, UV detection at 265 nm).

### Method CS5

Separation of the corresponding mixture (e.g. 0.35 mmol thereof) was performed using a Phenomenex Cellulose—4 column (20 μm, 350×76.5 mm), eluting with (n-Heptane 50% TEA 0.10%, EtOH 50% TEA 0.1%) (flow rate 24 L/h, UV detection at 265 nm).

### Method CS6

Separation of the corresponding mixture (e.g. 0.08 mmol thereof) was performed using a i-amylose-3 column (5 μm, 250×30 mm), eluting with (n-Heptane 80%, EtOH 20%)+TEA 0.10% (flow rate 40 mL/min, UV detection at 265 nm).

### Method CS7

Separation of the corresponding mixture (e.g. 0.26 mmol thereof) was performed using a Chiralpak AS column (10 μm, 250×4.6 mm), eluting with (MeOH 40%, CO₂60%)+TEA 0.10% (flow rate 120 mL/min, UV detection at 254 nm).

### Method CS8

Separation of the corresponding mixture (e.g. 0.09 mmol thereof) was performed using a Whelk 01 SS column (10 μm, 250×4.6 mm), eluting with (n-Heptane 80% TEA 0.1%, EtOH 20% TEA 0.1%) (flow rate 1 mL/min, UV detection at 265 nm).

### Method CS9

Separation of the corresponding mixture (e.g. 0.18 mmol thereof) was performed using a Chiralpak IJ column (5 μm, 250×30 mm), eluting with (MeOH 20%, CO₂ 80%) (flow rate 200 mL/min, UV detection at 265 nm).

### Method CS10

Separation of the corresponding mixture (e.g. 0.11 mmol thereof) was performed using Cellulose 4 column (5 μm, 250×4.6 mm), eluting with (n-Heptane 40% EtOH 60%)+0.1% TEA (flow rate 1 mL/min, UV detection at 265 nm).

### Method CS11

Separation of the corresponding mixture (e.g. 0.07 mmol thereof) was performed using a Chiralpak OZ-H column (5 μm, 250×30 mm), eluting with (n-Heptane 80% EtOH 20%)+0.10% TEA (flow rate 40 mL/min, UV detection at 265 nm).

### Method CS12

Separation of the corresponding mixture (e.g. 0.18 mmol thereof) was performed using a Chiralpak AY column (20 μm, 230×100 mm), eluting with (n-Heptane 80% EtOH 20%)+0.1% TEA (flow rate 400 mL/min, UV detection at 265 nm).

### Method CS13

Separation of the corresponding mixture (e.g. 0.28 mmol thereof) was performed using a Chiralpak OZ column (20 μm, 250×4.6 mm), eluting with (n-Heptane 20% EtOH 80%)+0.1% TEA (flow rate 1 mL/min, UV detection at 265 nm).

### Method CS14

Separation of the corresponding mixture (e.g. 0.075 mmol thereof) was performed using a Chiralpak IG column (5 μm, 250×30 mm), eluting with (n-Heptane 85%, Ethanol 15%)+0.10% TEA (flow rate 40 mL/min, UV detection at 265 nm).

### Method CS15

Separation of the corresponding mixture (e.g. 0.10 mmol thereof) was performed using a chiralpak AD column (20 m, 50×300 mm), eluting with (CO₂ (SFC) 85% MeOH 15%)+0.1% TEA (flow rate 200 mL/min, UV detection at 265 nm).

### Method CS16

Separation of the corresponding mixture (e.g. 0.16 mmol thereof) was performed using a XSELECT CSH prep C18 column (5 μm, 50×250 mm), eluting with (H₂O+0.1% HCOOH, MeCN, gradient) (flow rate 80 mL/min, UV detection at 265 nm).

### Method CS17

Separation of the corresponding mixture (e.g. 0.25 mmol thereof) was performed using a Chiracel OD-I column (20 μm, 76.5×350 mm), eluting with (n-Heptane 70/EtOH 30)+0.1% TEA (flow rate 400 mL/min, UV detection at 280 nm).

### Method CS18

Separation of the corresponding mixture (e.g. 0.18 mmol thereof) was performed using a Chiralpak IH column (5 m, 250×30 mm), eluting with (n-Heptane 82%, Ethanol 18%)+0.1% TEA (flow rate 40 mL/min, UV detection at 265 nm).

### Method CS19

Separation of the corresponding mixture (e.g. 0.36 mmol thereof) was performed using a Phenomenex lux amylose-1 column (5 μm, 250×30 mm), eluting with (n-Heptane 80%, EtOH 20%)+TEA 0.1% (flow rate 45 mL/min, UV detection at 265 nm).

### Method CS20

Separation of the corresponding mixture (e.g. 0.18 mmol thereof) was performed using a Chiralpak IF column (5 μm, 250×30 mm), eluting with (n-Heptane 80%, Ethanol 20%)+0.1% TEA (flow rate 1 mL/min, UV detection at 265 nm).

### Method CS21

Separation of the corresponding mixture was performed using a Chiralpak AS-H column (5 m, 20×250 mm), eluting with CO₂/30% (MeOH+0.1% TEA), flow rate 50 mL/min, UV detection at 254 nm, back pressure regulator 100 bar, T=40° C.

### Examples 1 to 461—Compounds

Table 1 below lists the compounds synthesized in the following synthetic examples.

TABLE 1

| Example No. | Structure | Name |
|---|---|---|
| 1 | 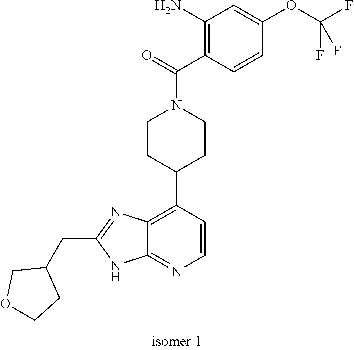 [SMILES: Nc1cc(OC(F)(F)F)ccc1C(=O)N1CCC(c2ccnc3[nH]c(CC4CCOC4)nc23)CC1] | [2-amino-4- (trifluoromethoxy)phenyl]-[4-[2- (tetrahydrofuran-3-ylmethyl)-3H- imidazo[4,5-b]pyridin-7-yl]-1- piperidyl]methanone, Isomer 1 |
| 2 | 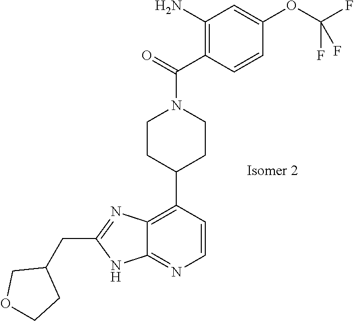 [SMILES: Nc1cc(OC(F)(F)F)ccc1C(=O)N1CCC(c2ccnc3[nH]c(CC4CCOC4)nc23)CC1] | [2-amino-4- (trifluoromethoxy)phenyl]-[4-[2- (tetrahydrofuran-3-ylmethyl)-3H- imidazo[4,5-b]pyridin-7-yl]-1- piperidyl]methanone, Isomer 2 |
| 3 | 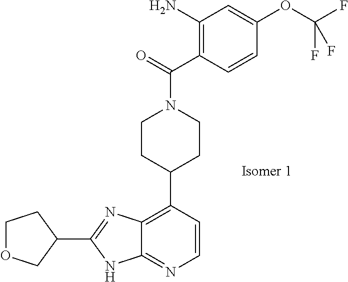 [SMILES: Nc1cc(OC(F)(F)F)ccc1C(=O)N1CCC(c2ccnc3[nH]c(C4CCOC4)nc23)CC1] | [2-amino-4- (trifluoromethoxy)phenyl]-[4-(2- tetrahydrofuran-3-yl-3H- imidazo[4,5-b]pyridin-7-yl)-1- piperidyl]methanone, Isomer 1 |
| 4 | 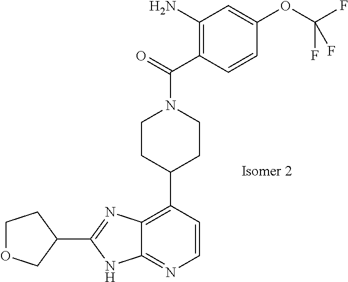 [SMILES: Nc1cc(OC(F)(F)F)ccc1C(=O)N1CCC(c2ccnc3[nH]c(C4CCOC4)nc23)CC1] | [2-amino-4- (trifluoromethoxy)phenyl]-[4-(2- tetrahydrofuran-3-yl-3H- imidazo[4,5-b]pyridin-7-yl)-1- piperidyl]methanone, Isomer 2 |
| 5 | 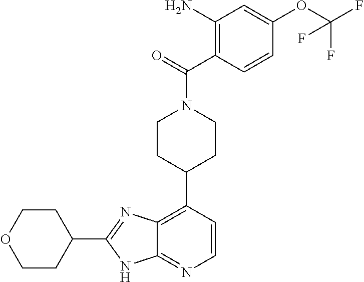 [SMILES: Nc1cc(OC(F)(F)F)ccc1C(=O)N1CCC(c2ccnc3[nH]c(C4CCOCC4)nc23)CC1] | [2-amino-4- (trifluoromethoxy)phenyl]-[4-(2- tetrahydropyran-4-yl-3H- imidazo[4,5-b]pyridin-7-yl)-1- piperidyl]methanone |
| 6 | 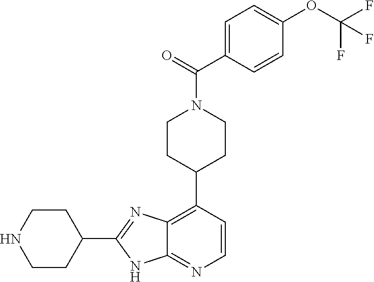 [SMILES: O=C(c1ccc(OC(F)(F)F)cc1)N1CCC(c2ccnc3[nH]c(C4CCNCC4)nc23)CC1] | [4-[2-(4-piperidyl)-3H-imidazo[4,5- b]pyridin-7-yl]-1-piperidyl]-[4- (trifluoromethoxy)phenyl]methanone |
| 7 | 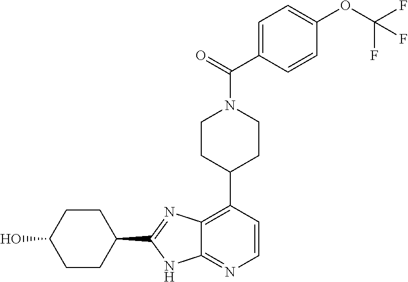 [SMILES: O=C(c1ccc(OC(F)(F)F)cc1)N1CCC(c2ccnc3[nH]c([C@H]4CC[C@H](O)CC4)nc23)CC1] | trans-[4-[2-(4-hydroxycyclohexyl)- 3H-imidazo[4,5-b]pyridin-7-yl]-1- piperidyl]-[4- (trifluoromethoxy)phenyl]methanone |
| 8 | 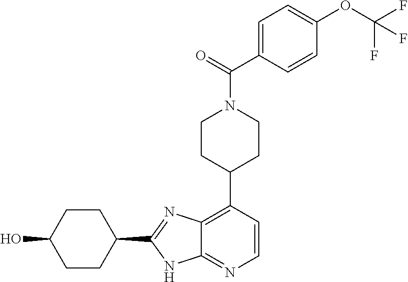 [SMILES: O=C(c1ccc(OC(F)(F)F)cc1)N1CCC(c2ccnc3[nH]c([C@H]4CC[C@@H](O)CC4)nc23)CC1] | cis-[4-[2-(4-hydroxycyclohexyl)-3H- imidazo[4,5-b]pyridin-7-yl]-1- piperidyl]-[4- (trifluoromethoxy)phenyl]methanone |
| 9 | 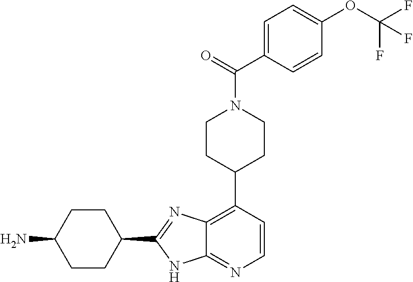 [SMILES: N[C@H]1CC[C@@H](c2nc3c(C4CCN(C(=O)c5ccc(OC(F)(F)F)cc5)CC4)ccnc3[nH]2)CC1] | cis-[4-[2-(4-aminocyclohexyl)-3H- imidazo[4,5-b]pyridin-7-yl]-1- piperidyl]-[4- (trifluoromethoxy)phenyl]methanone |
| 10 | 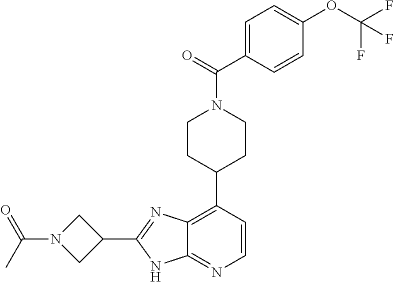 [SMILES: CC(=O)N1CC(c2nc3c(C4CCN(C(=O)c5ccc(OC(F)(F)F)cc5)CC4)ccnc3[nH]2)C1] | 1-[3-[7-[1-[4- (trifluoromethoxy)benzoyl]-4- piperidyl]-3H-imidazo[4,5-b]pyridin- 2-yl]azetidin-1-yl]ethanone |
| 11 | 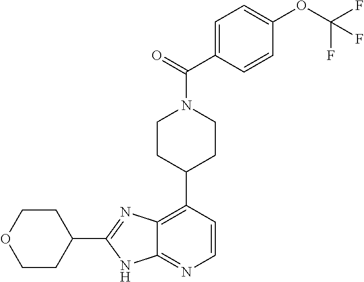 [SMILES: O=C(c1ccc(OC(F)(F)F)cc1)N1CCC(c2ccnc3[nH]c(C4CCOCC4)nc23)CC1] | [4-(2-tetrahydropyran-4-yl-3H- imidazo[4,5-b]pyridin-7-yl)-1- piperidyl]-[4- (trifluoromethoxy)phenyl]methanone |
| 12 | 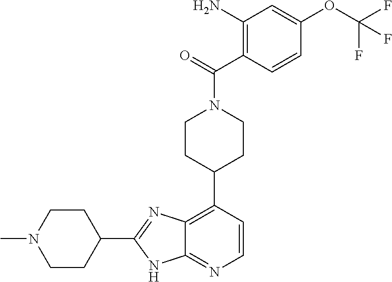 [SMILES: CN1CCC(c2nc3c(C4CCN(C(=O)c5ccc(OC(F)(F)F)cc5N)CC4)ccnc3[nH]2)CC1] | [2-amino-4- (trifluoromethoxy)phenyl]-[4-[2-(1- methyl-4-piperidyl)-3H-imidazo[4,5- b]pyridin-7-yl]-1- piperidyl]methanone |
| 13 | 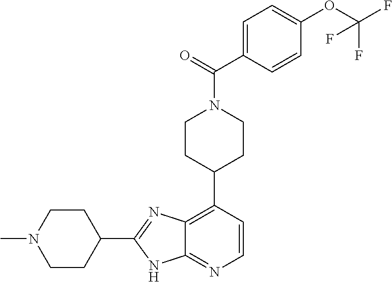 [SMILES: CN1CCC(c2nc3c(C4CCN(C(=O)c5ccc(OC(F)(F)F)cc5)CC4)ccnc3[nH]2)CC1] | [4-[2-(1-methyl-4-piperidyl)-3H- imidazo[4,5-b]pyridin-7-yl]-1- piperidyl]-[4- (trifluoromethoxy)phenyl]methanone |
| 14 | 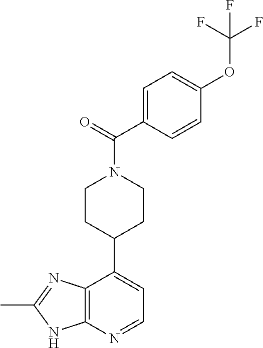 [SMILES: Cc1nc2c(C3CCN(C(=O)c4ccc(OC(F)(F)F)cc4)CC3)ccnc2[nH]1] | [4-(2-methyl-3H-imidazo[4,5- b]pyridin-7-yl)-1-piperidyl]-[4- (trifluoromethoxy)phenyl]methanone |
| 15 | 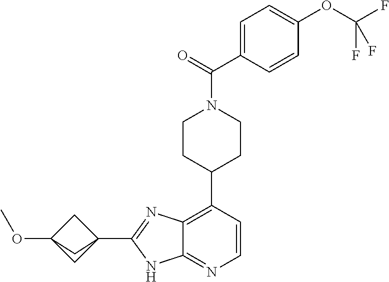 [SMILES: COC12CC(c3nc4c(C5CCN(C(=O)c6ccc(OC(F)(F)F)cc6)CC5)ccnc4[nH]3)(C1)C2] | [4-[2-(3-methoxy-1- bicyclo[1.1.1]pentanyl)-3H- imidazo[4,5-b]pyridin-7-yl]-1- piperidyl]-[4- (trifluoromethoxy)phenyl]methanone |
| 16 | 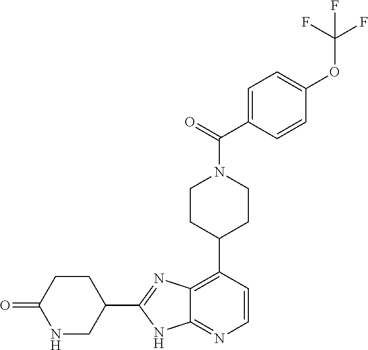 [SMILES: O=C1CCC(c2nc3c(C4CCN(C(=O)c5ccc(OC(F)(F)F)cc5)CC4)ccnc3[nH]2)CN1] | (rac)-5-[7-[1-[4- (trifluoromethoxy)benzoyl]-4- piperidyl]-3H-imidazo[4,5-b]pyridin- 2-yl]piperidin-2-one |
| 17 | 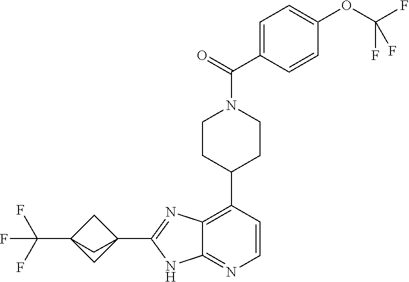 [SMILES: O=C(c1ccc(OC(F)(F)F)cc1)N1CCC(c2ccnc3[nH]c(C45CC(C(F)(F)F)(C4)C5)nc23)CC1] | [4-(trifluoromethoxy)phenyl]-[4-[2- [3-(trifluoromethyl)-1- bicyclo[1.1.1]pentanyl]-3H- imidazo[4,5-b]pyridin-7-yl]-1- piperidyl]methanone |
| 18 | 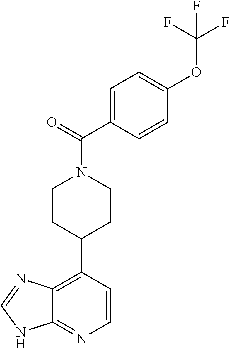 [SMILES: O=C(c1ccc(OC(F)(F)F)cc1)N1CCC(c2ccnc3[nH]cnc23)CC1] | [4-(3H-imidazo[4,5-b]pyridin-7-yl)- 1-piperidyl]-[4- (trifluoromethoxy)phenyl]methanone |
| 19 | 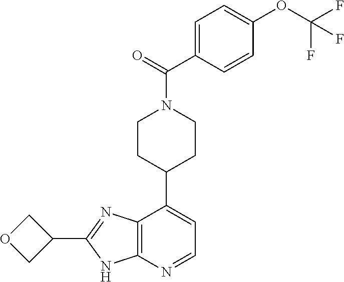 [SMILES: O=C(c1ccc(OC(F)(F)F)cc1)N1CCC(c2ccnc3[nH]c(C4COC4)nc23)CC1] | [4-[2-(oxetan-3-yl)-3H-imidazo[4,5- b]pyridin-7-yl]-1-piperidyl]-[4- (trifluoromethoxy)phenyl]methanone |
| 20 | 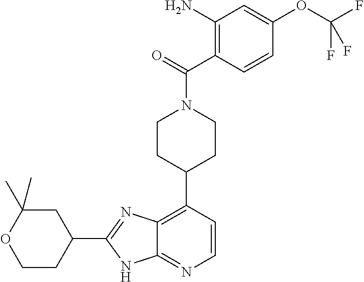 [SMILES: CC1(C)CC(c2nc3c(C4CCN(C(=O)c5ccc(OC(F)(F)F)cc5N)CC4)ccnc3[nH]2)CCO1] | (rac)-[2-amino-4- (trifluoromethoxy)phenyl]-[4-[2-(2,2- dimethyltetrahydropyran-4-yl)-3H- imidazo[4,5-b]pyridin-7-yl]-1- piperidyl]methanone |
| 21 | 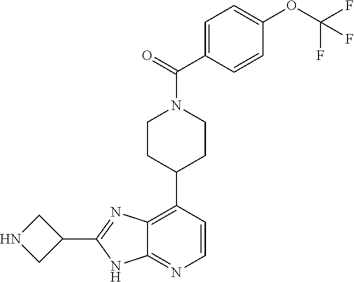 [SMILES: O=C(c1ccc(OC(F)(F)F)cc1)N1CCC(c2ccnc3[nH]c(C4CNC4)nc23)CC1] | [4-[2-(azetidin-3-yl)-3H-imidazo[4,5- b]pyridin-7-yl]-1-piperidyl]-[4- (trifluoromethoxy)phenyl]methanone |
| 22 | 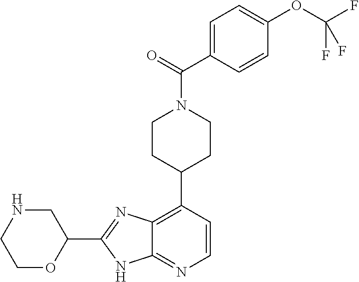 [SMILES: O=C(c1ccc(OC(F)(F)F)cc1)N1CCC(c2ccnc3[nH]c(C4CNCCO4)nc23)CC1] Isomer 1 | [4-(2-morpholin-2-yl-3H- imidazo[4,5-b]pyridin-7-yl)-1- piperidyl]-[4- (trifluoromethoxy)phenyl]methanone, Isomer 1 |
| 23 | 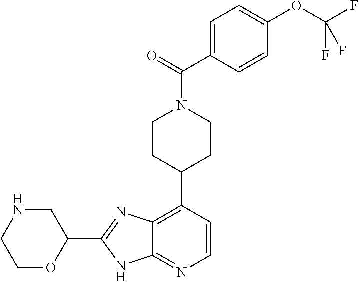 [SMILES: O=C(c1ccc(OC(F)(F)F)cc1)N1CCC(c2ccnc3[nH]c(C4CNCCO4)nc23)CC1] Isomer 2 | [4-(2-morpholin-2-yl-3H- imidazo[4,5-b]pyridin-7-yl)-1- piperidyl]-[4- (trifluoromethoxy)phenyl]methanone, Isomer 2 |
| 24 | 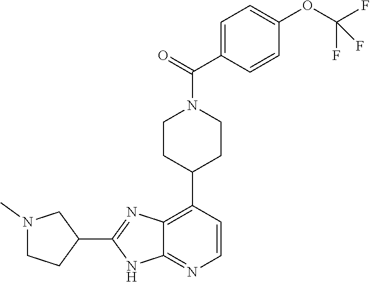 [SMILES: CN1CCC(c2nc3c(C4CCN(C(=O)c5ccc(OC(F)(F)F)cc5)CC4)ccnc3[nH]2)C1] Isomer 1 | [4-[2-(1-methylpyrrolidin-3-yl)-3H- imidazo[4,5-b]pyridin-7-yl]-1- piperidyl]-[4- (trifluoromethoxy)phenyl]methanone, Isomer 1 |
| 25 | 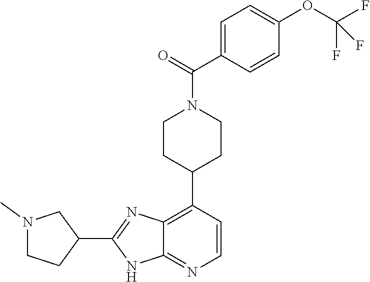 [SMILES: CN1CCC(c2nc3c(C4CCN(C(=O)c5ccc(OC(F)(F)F)cc5)CC4)ccnc3[nH]2)C1] Isomer 2 | [4-[2-(1-methylpyrrolidin-3-yl)-3H- imidazo[4,5-b]pyridin-7-yl]-1- piperidyl]-[4- (trifluoromethoxy)phenyl]methanone, Isomer 2 |
| 26 | 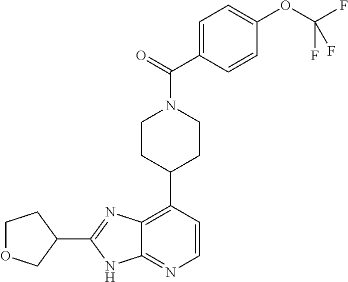 [SMILES: O=C(c1ccc(OC(F)(F)F)cc1)N1CCC(c2ccnc3[nH]c(C4CCOC4)nc23)CC1] Isomer 1 | [4-(2-tetrahydrofuran-3-yl-3H- imidazo[4,5-b]pyridin-7-yl)-1- piperidyl]-[4- (trifluoromethoxy)phenyl]methanone, Isomer 1 |
| 27 | 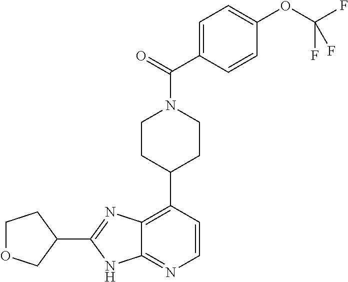 [SMILES: O=C(c1ccc(OC(F)(F)F)cc1)N1CCC(c2ccnc3[nH]c(C4CCOC4)nc23)CC1] Isomer 2 | [4-(2-tetrahydrofuran-3-yl-3H- imidazo[4,5-b]pyridin-7-yl)-1- piperidyl]-[4- (trifluoromethoxy)phenyl]methanone, Isomer 2 |
| 28 | 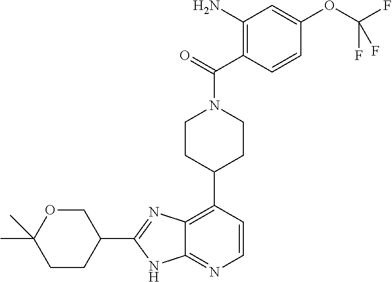 [SMILES: CC1(C)CCC(c2nc3c(C4CCN(C(=O)c5ccc(OC(F)(F)F)cc5N)CC4)ccnc3[nH]2)CO1] | (rac)-[2-amino-4- (trifluoromethoxy)phenyl]-[4-[2-(6,6- dimethyltetrahydropyran-3-yl)-3H- imidazo[4,5-b]pyridin-7-yl]-1- piperidyl]methanone |
| 29 | 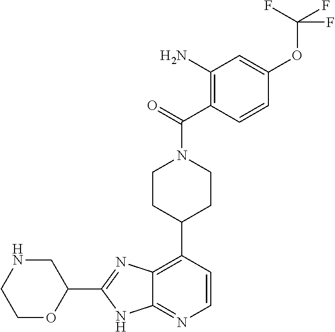 [SMILES: Nc1cc(OC(F)(F)F)ccc1C(=O)N1CCC(c2ccnc3[nH]c(C4CNCCO4)nc23)CC1] | (rac)-[2-amino-4- (trifluoromethoxy)phenyl]-[4-(2- morpholin-2-yl-3H-imidazo[4,5- b]pyridin-7-yl)-1- piperidyl]methanone |
| 30 | 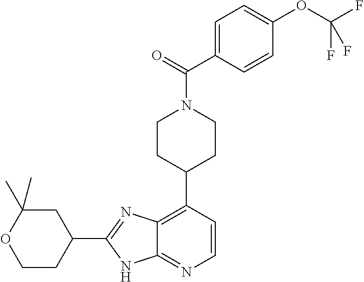 [SMILES: CC1(C)CC(c2nc3c(C4CCN(C(=O)c5ccc(OC(F)(F)F)cc5)CC4)ccnc3[nH]2)CCO1] | (rac)-[4-[2-(2,2- dimethyltetrahydropyran-4-yl)-3H- imidazo[4,5-b]pyridin-7-yl]-1- piperidyl]-[4- (trifluoromethoxy)phenyl]methanone |
| 31 | 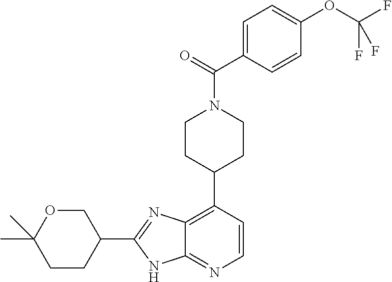 [SMILES: CC1(C)CCC(c2nc3c(C4CCN(C(=O)c5ccc(OC(F)(F)F)cc5)CC4)ccnc3[nH]2)CO1] | (rac)-[4-[2-(6,6- dimethyltetrahydropyran-3-yl)-3H- imidazo[4,5-b]pyridin-7-yl]-1- piperidyl]-[4- (trifluoromethoxy)phenyl]methanone |
| 32 | 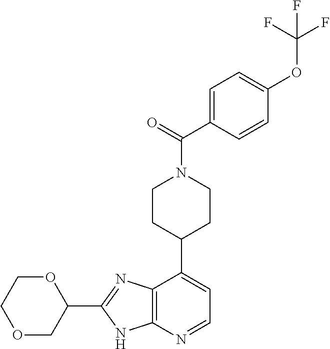 [SMILES: O=C(c1ccc(OC(F)(F)F)cc1)N1CCC(c2ccnc3[nH]c(C4COCCO4)nc23)CC1] Isomer 1 | [4-[2-(1,4-dioxan-2-yl)-3H- imidazo[4,5-b]pyridin-7-yl]-1- piperidyl]-[4- (trifluoromethoxy)phenyl]methanone, Isomer 1 |
| 33 | 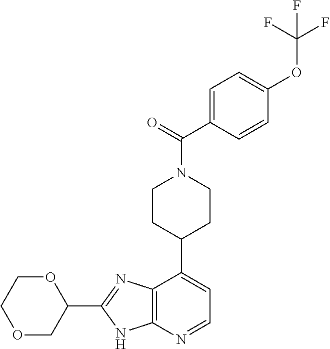 [SMILES: O=C(c1ccc(OC(F)(F)F)cc1)N1CCC(c2ccnc3[nH]c(C4COCCO4)nc23)CC1] Isomer 2 | [4-[2-(1,4-dioxan-2-yl)-3H- imidazo[4,5-b]pyridin-7-yl]-1- piperidyl]-[4- (trifluoromethoxy)phenyl]methanone, Isomer 2 |
| 34 | 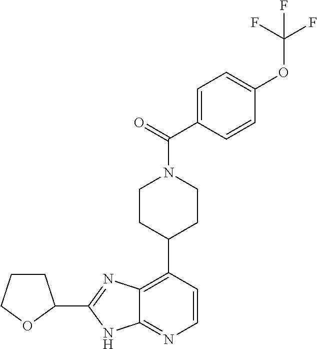 [SMILES: O=C(c1ccc(OC(F)(F)F)cc1)N1CCC(c2ccnc3[nH]c(C4CCCO4)nc23)CC1] | (rac)-[4-(2-tetrahydrofuran-2-yl-3H- imidazo[4,5-b]pyridin-7-yl)-1- piperidyl]-[4- (trifluoromethoxy)phenyl]methanone |
| 35 | 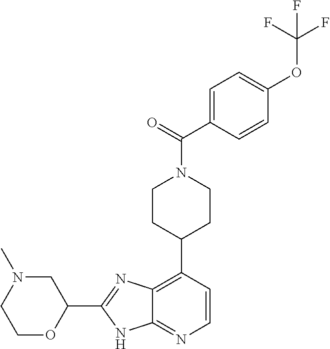 [SMILES: CN1CCOC(c2nc3c(C4CCN(C(=O)c5ccc(OC(F)(F)F)cc5)CC4)ccnc3[nH]2)C1] | (rac)-[4-[2-(4-methylmorpholin-2- yl)-3H-imidazo[4,5-b]pyridin-7-yl]- 1-piperidyl]-[4- (trifluoromethoxy)phenyl]methanone |
| 36 | 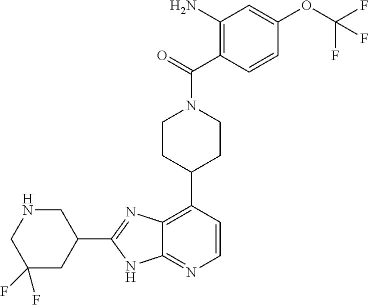 [SMILES: Nc1cc(OC(F)(F)F)ccc1C(=O)N1CCC(c2ccnc3[nH]c(C4CNCC(F)(F)C4)nc23)CC1] | (rac)-[2-amino-4- (trifluoromethoxy)phenyl]-[4-[2-(5,5- difluoro-3-piperidyl)-3H- imidazo[4,5-b]pyridin-7-yl]-1- piperidyl]methanone |
| 37 | 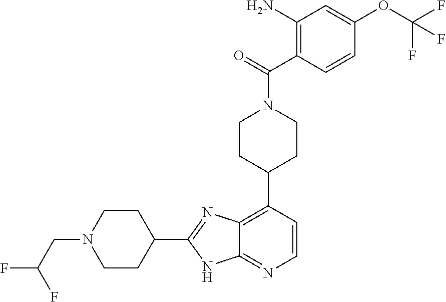 [SMILES: Nc1cc(OC(F)(F)F)ccc1C(=O)N1CCC(c2ccnc3[nH]c(C4CCN(CC(F)F)CC4)nc23)CC1] | [2-amino-4- (trifluoromethoxy)phenyl]-[4-[2-[1- (2,2-difluoroethyl)-4-piperidyl]-3H- imidazo[4,5-b]pyridin-7-yl]-1- piperidyl]methanone |
| 38 | 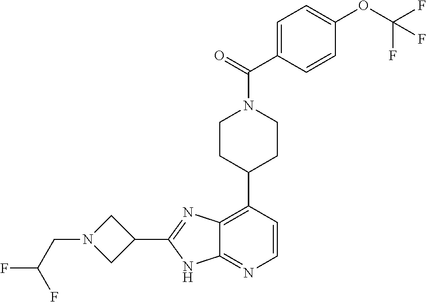 [SMILES: O=C(c1ccc(OC(F)(F)F)cc1)N1CCC(c2ccnc3[nH]c(C4CN(CC(F)F)C4)nc23)CC1] | [4-[2-[1-(2,2-difluoroethyl)azetidin- 3-yl]-3H-imidazo[4,5-b]pyridin-7- yl]-1-piperidyl]-[4- (trifluoromethoxy)phenyl]methanone |
| 39 | 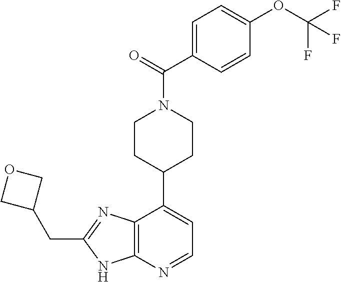 [SMILES: O=C(c1ccc(OC(F)(F)F)cc1)N1CCC(c2ccnc3[nH]c(CC4COC4)nc23)CC1] | [4-[2-(oxetan-3-ylmethyl)-3H- imidazo[4,5-b]pyridin-7-yl]-1- piperidyl]-[4- (trifluoromethoxy)phenyl]methanone |
| 40 | 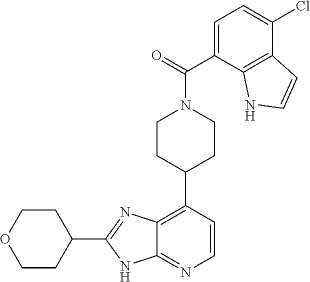 [SMILES: O=C(c1ccc(Cl)c2cc[nH]c12)N1CCC(c2ccnc3[nH]c(C4CCOCC4)nc23)CC1] | (4-chloro-1H-indol-7-yl)-[4-(2- tetrahydropyran-4-yl-3H- imidazo[4,5-b]pyridin-7-yl)-1- piperidyl]methanone |
| 41 | 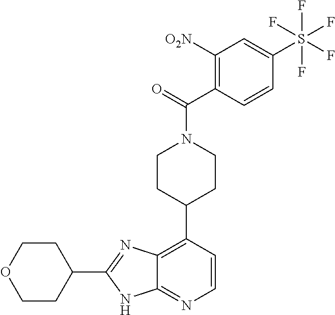 [SMILES: O=C(c1ccc(S(F)(F)(F)(F)F)cc1[N+](=O)[O-])N1CCC(c2ccnc3[nH]c(C4CCOCC4)nc23)CC1] | [2-nitro-4-(pentafluoro-λ⁶- sulfanyl)phenyl]-[4-(2- tetrahydropyran-4-yl-3H- imidazo[4,5-b]pyridin-7-yl)-1- piperidyl]methanone |
| 42 | 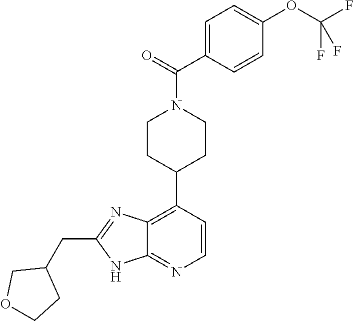 [SMILES: O=C(c1ccc(OC(F)(F)F)cc1)N1CCC(c2ccnc3[nH]c(CC4CCOC4)nc23)CC1] | (rac)-[4-[2-(tetrahydrofuran-3- ylmethyl)-3H-imidazo[4,5-b]pyridin- 7-yl]-1-piperidyl]-[4- (trifluoromethoxy)phenyl]methanone |
| 43 | 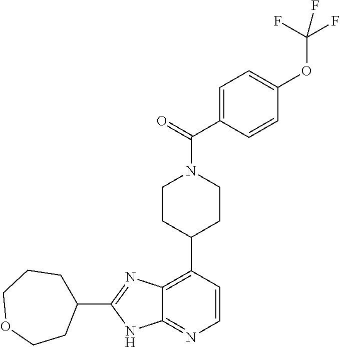 [SMILES: O=C(c1ccc(OC(F)(F)F)cc1)N1CCC(c2ccnc3[nH]c(C4CCCOCC4)nc23)CC1] | (rac)-[4-[2-(oxepan-4-yl)-3H- imidazo[4,5-b]pyridin-7-yl]-1- piperidyl]-[4- (trifluoromethoxy)phenyl]methanone |
| 44 | 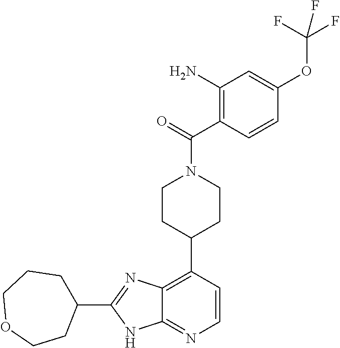 [SMILES: Nc1cc(OC(F)(F)F)ccc1C(=O)N1CCC(c2ccnc3[nH]c(C4CCCOCC4)nc23)CC1] | (rac)-[2-amino-4- (trifluoromethoxy)phenyl]-[4-[2- (oxepan-4-yl)-3H-imidazo[4,5- b]pyridin-7-yl]-1- piperidyl]methanone |
| 45 | 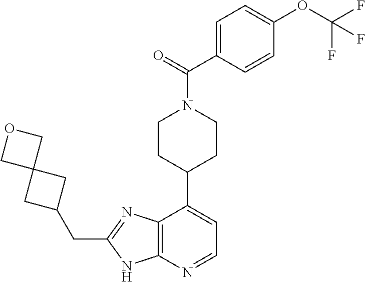 [SMILES: O=C(c1ccc(OC(F)(F)F)cc1)N1CCC(c2ccnc3[nH]c(CC4CC5(COC5)C4)nc23)CC1] | [4-[2-(2-oxaspiro[3.3]heptan-6- ylmethyl)-3H-imidazo[4,5-b]pyridin- 7-yl]-1-piperidyl]-[4- (trifluoromethoxy)phenyl]methanone |
| 46 | 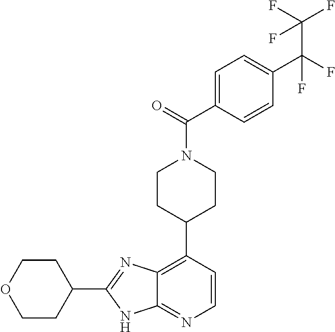 [SMILES: O=C(c1ccc(C(F)(F)C(F)(F)F)cc1)N1CCC(c2ccnc3[nH]c(C4CCOCC4)nc23)CC1] | [4-(1,1,2,2,2- pentafluoroethyl)phenyl]-[4-(2- tetrahydropyran-4-yl-3H- imidazo[4,5-b]pyridin-7-yl)-1- piperidyl]methanone |
| 47 | 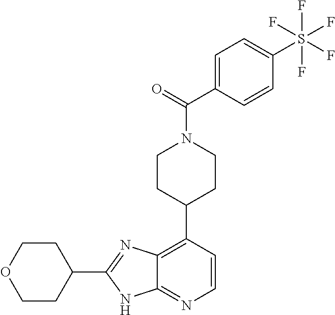 [SMILES: O=C(c1ccc(S(F)(F)(F)(F)F)cc1)N1CCC(c2ccnc3[nH]c(C4CCOCC4)nc23)CC1] | [4-(pentafluoro-λ⁶-sulfanyl)phenyl]- [4-(2-tetrahydropyran-4-yl-3H- imidazo[4,5-b]pyridin-7-yl)-1- piperidyl]methanone |
| 48 | 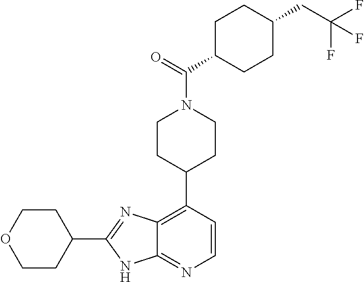 [SMILES: O=C([C@H]1CC[C@@H](CC(F)(F)F)CC1)N1CCC(c2ccnc3[nH]c(C4CCOCC4)nc23)CC1] | (cis)-[4-(2-tetrahydropyran-4-yl-3H- imidazo[4,5-b]pyridin-7-yl)-1- piperidyl]-[4-(2,2,2- trifluoroethyl)cyclohexyl]methanone |
| 49 | 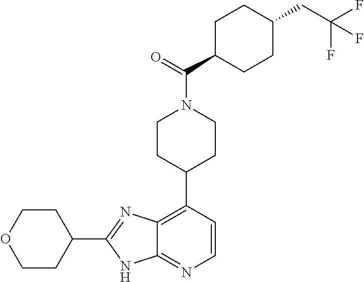 [SMILES: O=C([C@H]1CC[C@H](CC(F)(F)F)CC1)N1CCC(c2ccnc3[nH]c(C4CCOCC4)nc23)CC1] | (trans)-[4-(2-tetrahydropyran-4-yl- 3H-imidazo[4,5-b]pyridin-7-yl)-1- piperidyl]-[4-(2,2,2- trifluoroethyl)cyclohexyl]methanone |
| 50 | 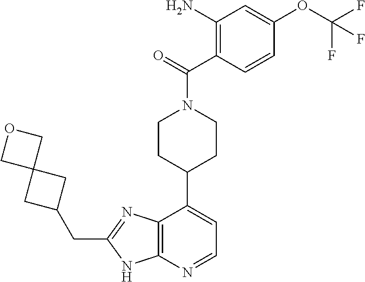 [SMILES: Nc1cc(OC(F)(F)F)ccc1C(=O)N1CCC(c2ccnc3[nH]c(CC4CC5(COC5)C4)nc23)CC1] | [2-amino-4- (trifluoromethoxy)phenyl]-[4-[2-(2- oxaspiro[3.3]heptan-6-ylmethyl)-3H- imidazo[4,5-b]pyridin-7-yl]-1- piperidyl]methanone |
| 51 | 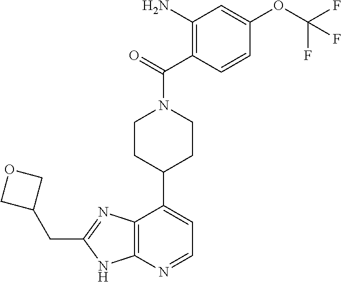 [SMILES: Nc1cc(OC(F)(F)F)ccc1C(=O)N1CCC(c2ccnc3[nH]c(CC4COC4)nc23)CC1] | [2-amino-4- (trifluoromethoxy)phenyl]-[4-[2- (oxetan-3-ylmethyl)-3H-imidazo[4,5- b]pyridin-7-yl]-1- piperidyl]methanone |
| 52 | 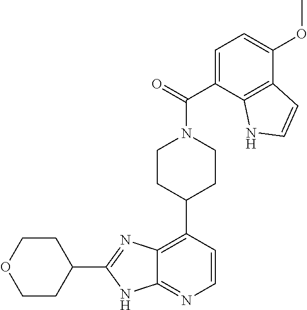 [SMILES: COc1ccc(C(=O)N2CCC(c3ccnc4[nH]c(C5CCOCC5)nc34)CC2)c2[nH]ccc12] | (4-methoxy-1H-indol-7-yl)-[4-(2- tetrahydropyran-4-yl-3H- imidazo[4,5-b]pyridin-7-yl)-1- piperidyl]methanone |
| 53 | 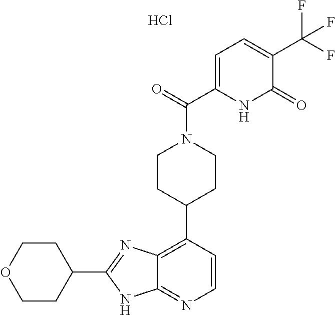 [SMILES: O=C(c1ccc(C(F)(F)F)c(=O)[nH]1)N1CCC(c2ccnc3[nH]c(C4CCOCC4)nc23)CC1 ; Cl] | 6-[4-(2-tetrahydropyran-4-yl-3H- imidazo[4,5-b]pyridin-7- yl)piperidine-1-carbonyl]-3- (trifluoromethyl)-1H-pyridin-2-one, hydrochloride |
| 54 | 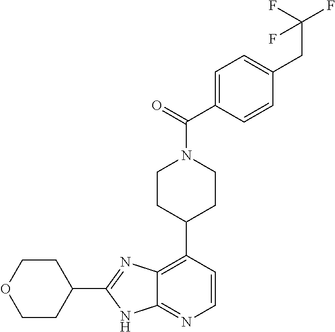 [SMILES: O=C(c1ccc(CC(F)(F)F)cc1)N1CCC(c2ccnc3[nH]c(C4CCOCC4)nc23)CC1] | [4-(2-tetrahydropyran-4-yl-3H- imidazo[4,5-b]pyridin-7-yl)-1- piperidyl]-[4-(2,2,2- trifluoroethyl)phenyl]methanone |
| 55 | 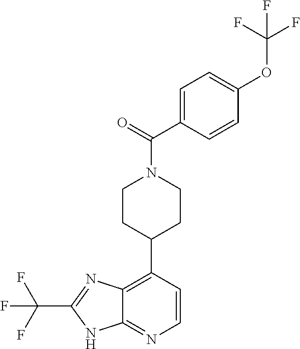 [SMILES: O=C(c1ccc(OC(F)(F)F)cc1)N1CCC(c2ccnc3[nH]c(C(F)(F)F)nc23)CC1] | [4-(trifluoromethoxy)phenyl]-[4-[2- (trifluoromethyl)-3H-imidazo[4,5- b]pyridin-7-yl]-1- piperidyl]methanone |
| 56 | 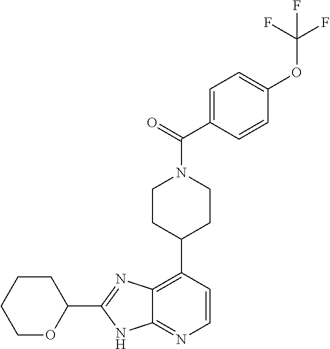 [SMILES: O=C(c1ccc(OC(F)(F)F)cc1)N1CCC(c2ccnc3[nH]c(C4CCCCO4)nc23)CC1] | (rac)-[4-(2-tetrahydropyran-2-yl-3H- imidazo[4,5-b]pyridin-7-yl)-1- piperidyl]-[4- (trifluoromethoxy)phenyl]methanone |
| 57 | 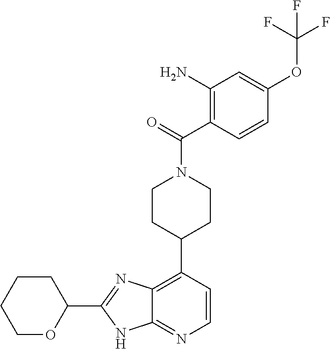 [SMILES: Nc1cc(OC(F)(F)F)ccc1C(=O)N1CCC(c2ccnc3[nH]c(C4CCCCO4)nc23)CC1] | (rac)-[2-amino-4- (trifluoromethoxy)phenyl]-[4-(2- tetrahydropyran-2-yl-3H- imidazo[4,5-b]pyridin-7-yl)-1- piperidyl]methanone |
| 58 | 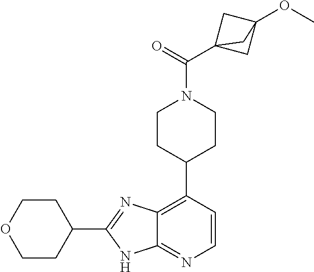 [SMILES: COC12CC(C(=O)N3CCC(c4ccnc5[nH]c(C6CCOCC6)nc45)CC3)(C1)C2] | (3-methoxy-1- bicyclo[1.1.1]pentanyl)-[4-(2- tetrahydropyran-4-yl-3H- imidazo[4,5-b]pyridin-7-yl)-1- piperidyl]methanone |
| 59 | 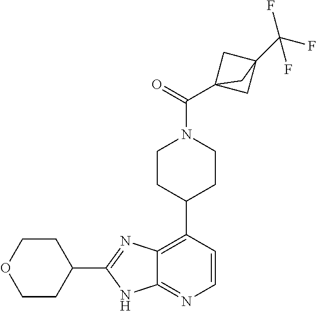 [SMILES: O=C(N1CCC(c2ccnc3[nH]c(C4CCOCC4)nc23)CC1)C12CC(C(F)(F)F)(C1)C2] | [4-(2-tetrahydropyran-4-yl-3H- imidazo[4,5-b]pyridin-7-yl)-1- piperidyl]-[3-(trifluoromethyl)-1- bicyclo[1.1.1]pentanyl]methanone |
| 60 | 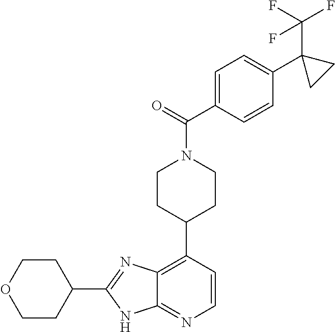 [SMILES: O=C(c1ccc(C2(C(F)(F)F)CC2)cc1)N1CCC(c2ccnc3[nH]c(C4CCOCC4)nc23)CC1] | [4-(2-tetrahydropyran-4-yl-3H- imidazo[4,5-b]pyridin-7-yl)-1- piperidyl]-[4-[1- (trifluoromethyl)cyclopropyl]phenyl] methanone |
| 61 | 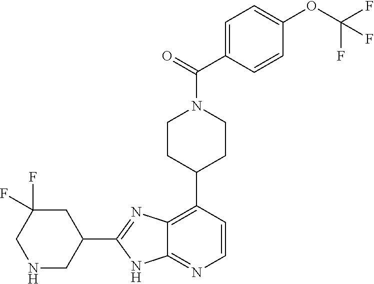 [SMILES: O=C(c1ccc(OC(F)(F)F)cc1)N1CCC(c2ccnc3[nH]c(C4CNCC(F)(F)C4)nc23)CC1] | (rac)-[4-[2-(5,5-difluoro-3-piperidyl)- 3H-imidazo[4,5-b]pyridin-7-yl]-1- piperidyl]-[4- (trifluoromethoxy)phenyl]methanone |
| 62 | 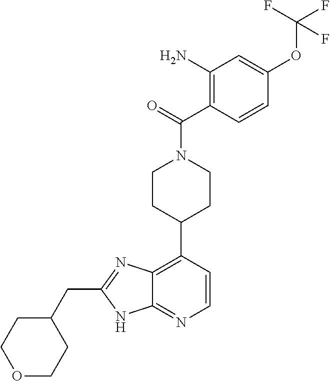 [SMILES: Nc1cc(OC(F)(F)F)ccc1C(=O)N1CCC(c2ccnc3[nH]c(CC4CCOCC4)nc23)CC1] | [2-amino-4- (trifluoromethoxy)phenyl]-[4-[2- (tetrahydropyran-4-ylmethyl)-3H- imidazo[4,5-b]pyridin-7-yl]-1- piperidyl]methanone |
| 63 | 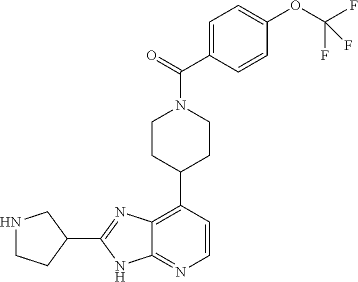 [SMILES: O=C(c1ccc(OC(F)(F)F)cc1)N1CCC(c2ccnc3[nH]c(C4CCNC4)nc23)CC1] | (rac)-[4-(2-pyrrolidin-3-yl-3H- imidazo[4,5-b]pyridin-7-yl)-1- piperidyl]-[4- (trifluoromethoxy)phenyl]methanone |
| 64 | 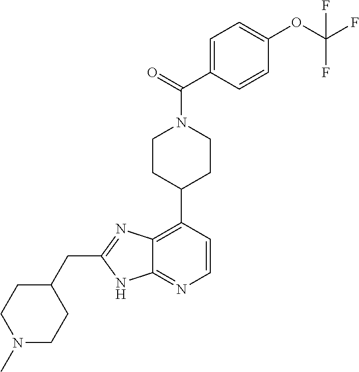 [SMILES: CN1CCC(Cc2nc3c(C4CCN(C(=O)c5ccc(OC(F)(F)F)cc5)CC4)ccnc3[nH]2)CC1] | [4-[2-[(1-methyl-4-piperidyl)methyl]- 3H-imidazo[4,5-b]pyridin-7-yl]-1- piperidyl]-[4- (trifluoromethoxy)phenyl]methanone |
| 65 | 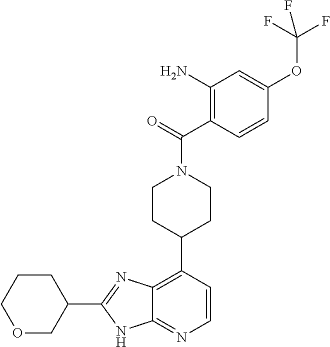 [SMILES: Nc1cc(OC(F)(F)F)ccc1C(=O)N1CCC(c2ccnc3[nH]c(C4CCCOC4)nc23)CC1] | (rac)-[2-amino-4- (trifluoromethoxy)phenyl]-[4-(2- tetrahydropyran-3-yl-3H- imidazo[4,5-b]pyridin-7-yl)-1- piperidyl]methanone |
| 66 | 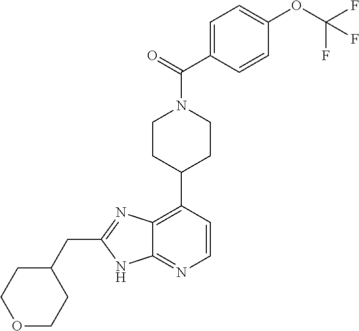 [SMILES: O=C(c1ccc(OC(F)(F)F)cc1)N1CCC(c2ccnc3[nH]c(CC4CCOCC4)nc23)CC1] | [4-[2-(tetrahydropyran-4-ylmethyl)- 3H-imidazo[4,5-b]pyridin-7-yl]-1- piperidyl]-[4- (trifluoromethoxy)phenyl]methanone |
| 67 | 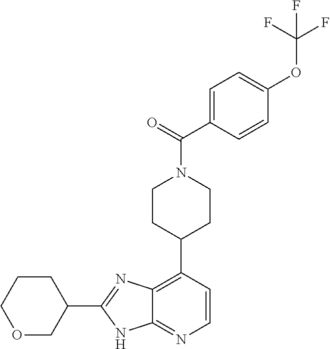 [SMILES: O=C(c1ccc(OC(F)(F)F)cc1)N1CCC(c2ccnc3[nH]c(C4CCCOC4)nc23)CC1] | (rac)-[4-(2-tetrahydropyran-3-yl-3H- imidazo[4,5-b]pyridin-7-yl)-1- piperidyl]-[4- (trifluoromethoxy)phenyl]methanone |
| 68 | 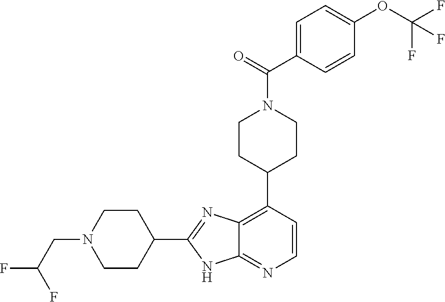 [SMILES: O=C(c1ccc(OC(F)(F)F)cc1)N1CCC(c2ccnc3[nH]c(C4CCN(CC(F)F)CC4)nc23)CC1] | [4-[2-[1-(2,2-difluoroethyl)-4- piperidyl]-3H-imidazo[4,5-b]pyridin- 7-yl]-1-piperidyl]-[4- (trifluoromethoxy)phenyl]methanone |
| 69 | 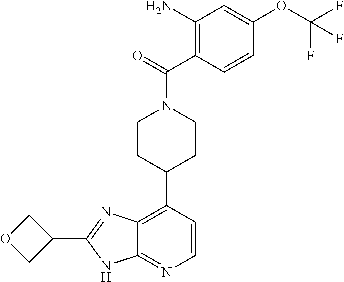 [SMILES: Nc1cc(OC(F)(F)F)ccc1C(=O)N1CCC(c2ccnc3[nH]c(C4COC4)nc23)CC1] | [2-amino-4- (trifluoromethoxy)phenyl]-[4-[2- (oxetan-3-yl)-3H-imidazo[4,5- b]pyridin-7-yl]-1- piperidyl]methanone |
| 70 | 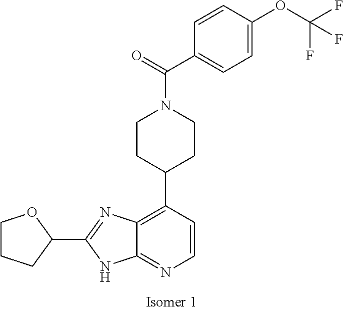 [SMILES: O=C(c1ccc(OC(F)(F)F)cc1)N1CCC(c2ccnc3[nH]c(C4CCCO4)nc23)CC1] | [4-[2-[tetrahydrofuran-2-yl]-3H- imidazo[4,5-b]pyridin-7-yl]-1- piperidyl]-[4- (trifluoromethoxy)phenyl]methanone, Isomer 1 |
| 71 | 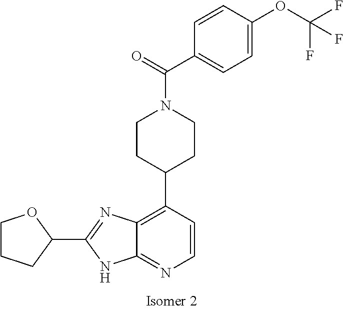 [SMILES: O=C(c1ccc(OC(F)(F)F)cc1)N1CCC(c2ccnc3[nH]c(C4CCCO4)nc23)CC1] | [4-[2-[tetrahydrofuran-2-yl]-3H- imidazo[4,5-b]pyridin-7-yl]-1- piperidyl]-[4- (trifluoromethoxy)phenyl]methanone, Isomer 2 |
| 72 | 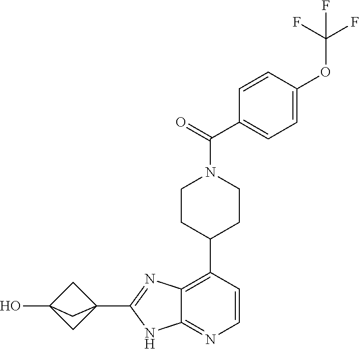 [SMILES: O=C(c1ccc(OC(F)(F)F)cc1)N1CCC(c2ccnc3[nH]c(C45CC(O)(C4)C5)nc23)CC1] | [4-[2-(3-hydroxy-1- bicyclo[1.1.1]pentanyl)-3H- imidazo[4,5-b]pyridin-7-yl]-1- piperidyl]-[4- (trifluoromethoxy)phenyl]methanone |
| 73 | 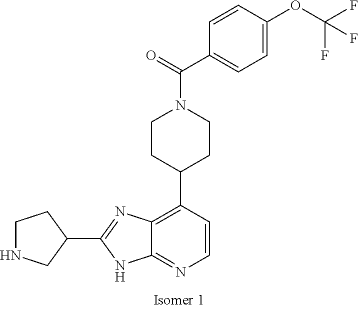 [SMILES: O=C(c1ccc(OC(F)(F)F)cc1)N1CCC(c2ccnc3[nH]c(C4CCNC4)nc23)CC1] | [4-[2-[pyrrolidin-3-yl]-3H- imidazo[4,5-b]pyridin-7-yl]-1- piperidyl]-[4- (trifluoromethoxy)phenyl]methanone, Isomer 1 |
| 74 | 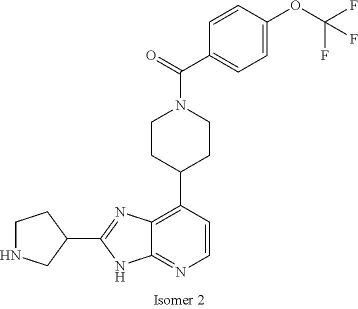 [SMILES: O=C(c1ccc(OC(F)(F)F)cc1)N1CCC(c2ccnc3[nH]c(C4CCNC4)nc23)CC1] | [4-[2-[pyrrolidin-3-yl]-3H- imidazo[4,5-b]pyridin-7-yl]-1- piperidyl]-[4- (trifluoromethoxy)phenyl]methanone, Isomer 2 |
| 75 | 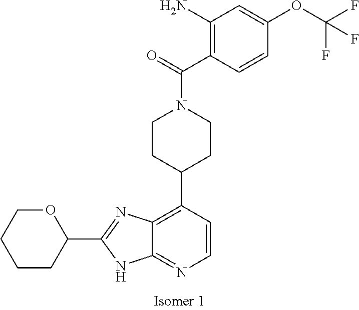 [SMILES: Nc1cc(OC(F)(F)F)ccc1C(=O)N1CCC(c2ccnc3[nH]c(C4CCCCO4)nc23)CC1] | [2-amino-4- (trifluoromethoxy)phenyl]-[4-[2- [tetrahydropyran-2-yl]-3H- imidazo[4,5-b]pyridin-7-yl]-1- piperidyl]methanone, Isomer 1 |
| 76 | 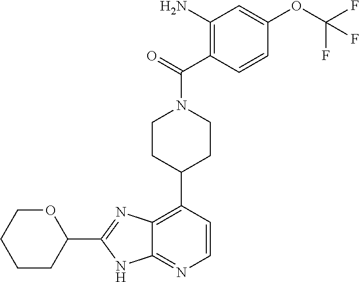 [SMILES: Nc1cc(OC(F)(F)F)ccc1C(=O)N1CCC(c2ccnc3[nH]c(C4CCCCO4)nc23)CC1] Isomer 2 | [2-amino-4- (trifluoromethoxy)phenyl]-[4-[2- [tetrahydropyran-2-yl]-3H- imidazo[4,5-b]pyridin-7-yl]-1- piperidyl]methanone, Isomer 2 |
| 77 | 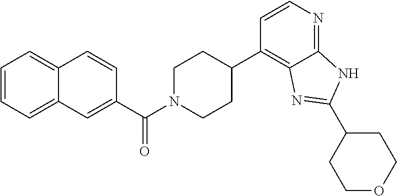 [SMILES: O=C(c1ccc2ccccc2c1)N1CCC(c2ccnc3[nH]c(C4CCOCC4)nc23)CC1] | 2-naphthyl-[4-(2-tetrahydropyran-4- yl-3H-imidazo[4,5-b]pyridin-7-yl)-1- piperidyl]methanone |
| 78 | 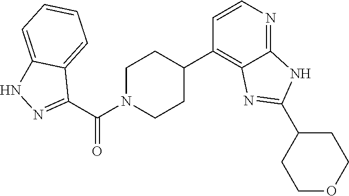 [SMILES: O=C(c1n[nH]c2ccccc12)N1CCC(c2ccnc3[nH]c(C4CCOCC4)nc23)CC1] | 1H-indazol-3-yl-[4-(2- tetrahydropyran-4-yl-3H- imidazo[4,5-b]pyridin-7-yl)-1- piperidyl]methanone |
| 79 | 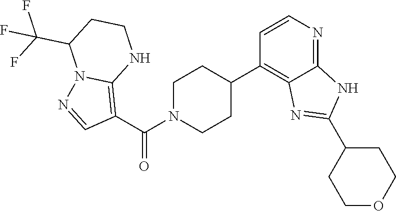 [SMILES: O=C(c1cnn2c1NCCC2C(F)(F)F)N1CCC(c2ccnc3[nH]c(C4CCOCC4)nc23)CC1] | [4-(2-tetrahydropyran-4-yl-3H- imidazo[4,5-b]pyridin-7-yl)-1- piperidyl]-[7-(trifluoromethyl)- 4,5,6,7-tetrahydropyrazolo[1,5- a]pyrimidin-3-yl]methanone |
| 80 | 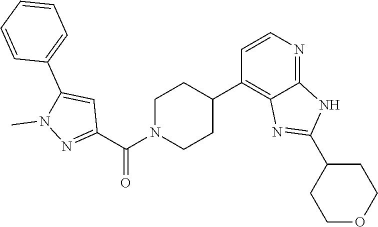 [SMILES: Cn1nc(C(=O)N2CCC(c3ccnc4[nH]c(C5CCOCC5)nc34)CC2)cc1-c1ccccc1] | (1-methyl-5-phenyl-pyrazol-3-yl)-[4- (2-tetrahydropyran-4-yl-3H- imidazo[4,5-b]pyridin-7-yl)-1- piperidyl]methanone |
| 81 | 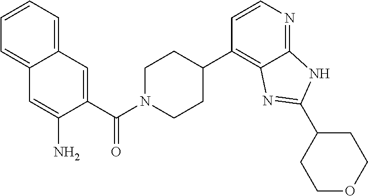 [SMILES: Nc1cc2ccccc2cc1C(=O)N1CCC(c2ccnc3[nH]c(C4CCOCC4)nc23)CC1] | (3-amino-2-naphthyl)-[4-(2- tetrahydropyran-4-yl-3H- imidazo[4,5-b]pyridin-7-yl)-1- piperidyl]methanone |
| 82 | 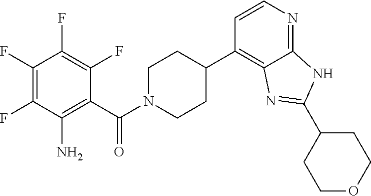 [SMILES: Nc1c(F)c(F)c(F)c(F)c1C(=O)N1CCC(c2ccnc3[nH]c(C4CCOCC4)nc23)CC1] | (2-amino-3,4,5,6-tetrafluoro-phenyl)- [4-(2-tetrahydropyran-4-yl-3H- imidazo[4,5-b]pyridin-7-yl)-1- piperidyl]methanone |
| 83 | 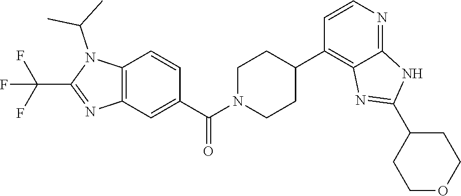 [SMILES: CC(C)n1c(C(F)(F)F)nc2cc(C(=O)N3CCC(c4ccnc5[nH]c(C6CCOCC6)nc45)CC3)ccc21] | [1-isopropyl-2- (trifluoromethyl)benzimidazol-5-yl]- [4-(2-tetrahydropyran-4-yl-3H- imidazo[4,5-b]pyridin-7-yl)-1- piperidyl]methanone |
| 84 | 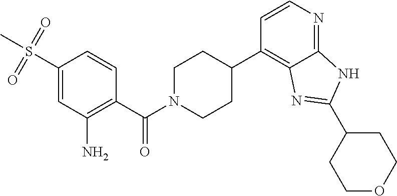 [SMILES: CS(=O)(=O)c1ccc(C(=O)N2CCC(c3ccnc4[nH]c(C5CCOCC5)nc34)CC2)c(N)c1] | (2-amino-4-methylsulfonyl-phenyl)- [4-(2-tetrahydropyran-4-yl-3H- imidazo[4,5-b]pyridin-7-yl)-1- piperidyl]methanone |
| 85 | 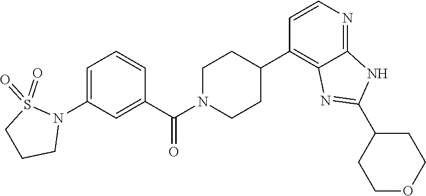 [SMILES: O=C(c1cccc(N2CCCS2(=O)=O)c1)N1CCC(c2ccnc3[nH]c(C4CCOCC4)nc23)CC1] | [3-(1,1-dioxo-1,2-thiazolidin-2- yl)phenyl]-[4-(2-tetrahydropyran-4- yl-3H-imidazo[4,5-b]pyridin-7-yl)-1- piperidyl]methanone |
| 86 | 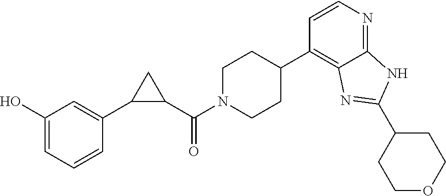 [SMILES: O=C(C1CC1c1cccc(O)c1)N1CCC(c2ccnc3[nH]c(C4CCOCC4)nc23)CC1] | [2-(3-hydroxyphenyl)cyclopropyl]- [4-(2-tetrahydropyran-4-yl-3H- imidazo[4,5-b]pyridin-7-yl)-1- piperidyl]methanone |
| 87 | 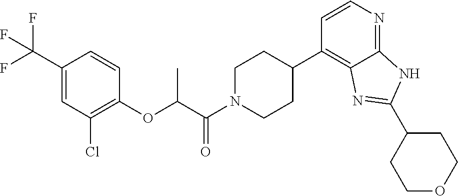 [SMILES: CC(Oc1ccc(C(F)(F)F)cc1Cl)C(=O)N1CCC(c2ccnc3[nH]c(C4CCOCC4)nc23)CC1] | 2-[2-chloro-4- (trifluoromethyl)phenoxy]-1-[4-(2- tetrahydropyran-4-yl-3H- imidazo[4,5-b]pyridin-7-yl)-1- piperidyl]propan-1-one |
| 88 | 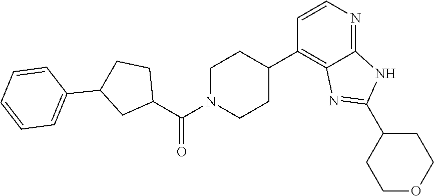 [SMILES: O=C(C1CCC(c2ccccc2)C1)N1CCC(c2ccnc3[nH]c(C4CCOCC4)nc23)CC1] | (3-phenylcyclopentyl)-[4-(2- tetrahydropyran-4-yl-3H- imidazo[4,5-b]pyridin-7-yl)-1- piperidyl]methanone |
| 89 | 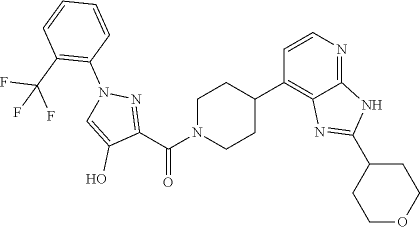 [SMILES: O=C(c1nn(-c2ccccc2C(F)(F)F)cc1O)N1CCC(c2ccnc3[nH]c(C4CCOCC4)nc23)CC1] | [4-hydroxy-1-[2- (trifluoromethyl)phenyl]pyrazol-3- yl]-[4-(2-tetrahydropyran-4-yl-3H- imidazo[4,5-b]pyridin-7-yl)-1- piperidyl]methanone |
| 90 | 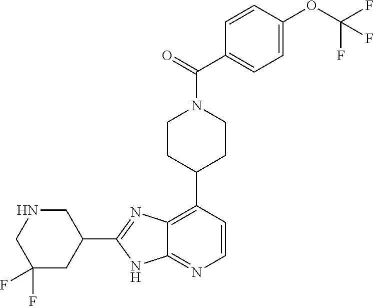 [SMILES: O=C(c1ccc(OC(F)(F)F)cc1)N1CCC(c2ccnc3[nH]c(C4CNCC(F)(F)C4)nc23)CC1] Isomer 1 | [4-[2-[5,5-difluoro-3-piperidyl]-3H- imidazo[4,5-b]pyridin-7-yl]-1- piperidyl]-[4- (trifluoromethoxy)phenyl]methanone, Isomer 1 |
| 91 | 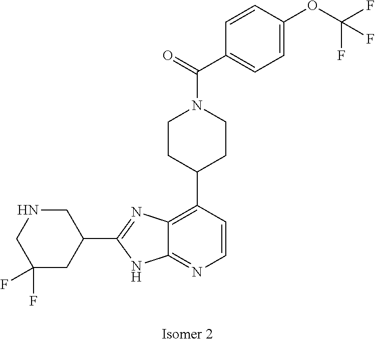 [SMILES: O=C(c1ccc(OC(F)(F)F)cc1)N1CCC(c2ccnc3[nH]c(C4CNCC(F)(F)C4)nc23)CC1] | [4-[2-[5,5-difluoro-3-piperidyl]-3H- imidazo[4,5-b]pyridin-7-yl]-1- piperidyl]-[4- (trifluoromethoxy)phenyl]methanone, Isomer 2 |
| 92 | 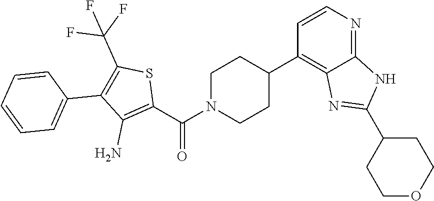 [SMILES: Nc1c(C(=O)N2CCC(c3ccnc4[nH]c(C5CCOCC5)nc34)CC2)sc(C(F)(F)F)c1-c1ccccc1] | [3-amino-4-phenyl-5- (trifluoromethyl)-2-thienyl]-[4-(2- tetrahydropyran-4-yl-3H- imidazo[4,5-b]pyridin-7-yl)-1- piperidyl]methanone |
| 93 | 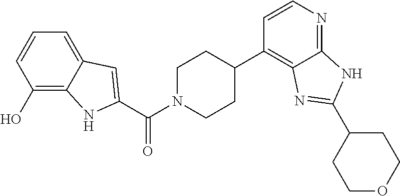 [SMILES: O=C(c1cc2cccc(O)c2[nH]1)N1CCC(c2ccnc3[nH]c(C4CCOCC4)nc23)CC1] | (7-hydroxy-1H-indol-2-yl)-[4-(2- tetrahydropyran-4-yl-3H- imidazo[4,5-b]pyridin-7-yl)-1- piperidyl]methanone |
| 94 | 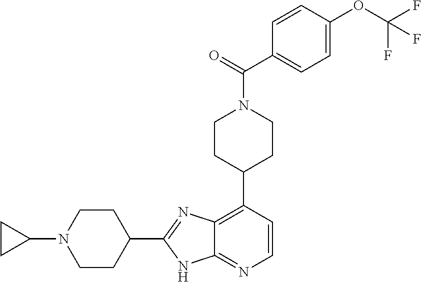 [SMILES: O=C(c1ccc(OC(F)(F)F)cc1)N1CCC(c2ccnc3[nH]c(C4CCN(C5CC5)CC4)nc23)CC1] | [4-[2-(1-cyclopropyl-4-piperidyl)- 3H-imidazo[4,5-b]pyridin-7-yl]-1- piperidyl]-[4- (trifluoromethoxy)phenyl]methanone |
| 95 | 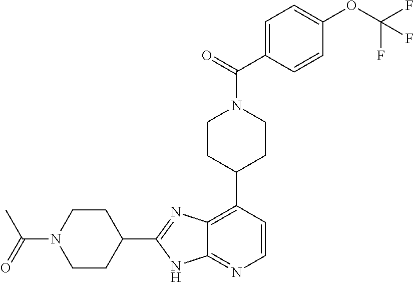 [SMILES: CC(=O)N1CCC(c2nc3c(C4CCN(C(=O)c5ccc(OC(F)(F)F)cc5)CC4)ccnc3[nH]2)CC1] | 1-[4-[7-[1-[4- (trifluoromethoxy)benzoyl]-4- piperidyl]-3H-imidazo[4,5-b]pyridin- 2-yl]-1-piperidyl]ethanone |
| 96 | 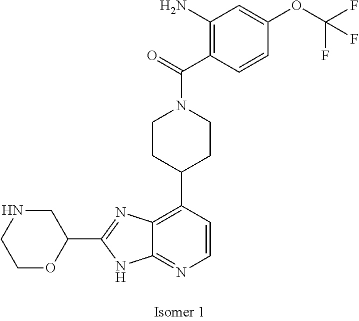 [SMILES: Nc1cc(OC(F)(F)F)ccc1C(=O)N1CCC(c2ccnc3[nH]c(C4CNCCO4)nc23)CC1] | [2-amino-4- (trifluoromethoxy)phenyl]-[4-[2- [morpholin-2-yl]-3H-imidazo[4,5- b]pyridin-7-yl]-1- piperidyl]methanone, Isomer 1 |
| 97 | 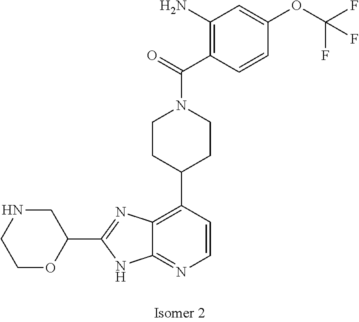 [SMILES: Nc1cc(OC(F)(F)F)ccc1C(=O)N1CCC(c2ccnc3[nH]c(C4CNCCO4)nc23)CC1] | [2-amino-4- (trifluoromethoxy)phenyl]-[4-[2- [morpholin-2-yl]-3H-imidazo[4,5- b]pyridin-7-yl]-1- piperidyl]methanone, Isomer 2 |
| 98 | 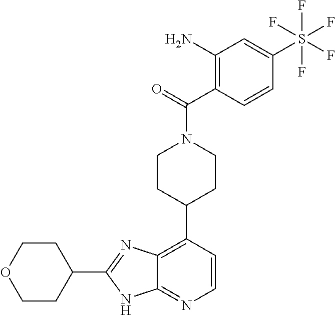 [SMILES: Nc1cc(S(F)(F)(F)(F)F)ccc1C(=O)N1CCC(c2ccnc3[nH]c(C4CCOCC4)nc23)CC1] | [2-amino-4-(pentafluoro-λ⁶- sulfanyl)phenyl]-[4-(2- tetrahydropyran-4-yl-3H- imidazo[4,5-b]pyridin-7-yl)-1- piperidyl]methanone |
| 99 | 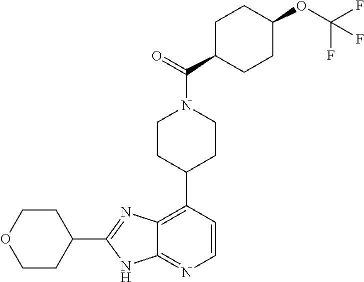 [SMILES: O=C([C@H]1CC[C@@H](OC(F)(F)F)CC1)N1CCC(c2ccnc3[nH]c(C4CCOCC4)nc23)CC1] | (cis)-[4-(2-tetrahydropyran-4-yl-3H- imidazo[4,5-b]pyridin-7-yl)-1- piperidyl]-[4- (trifluoromethoxy)cyclohexyl] methanone |
| 100 | 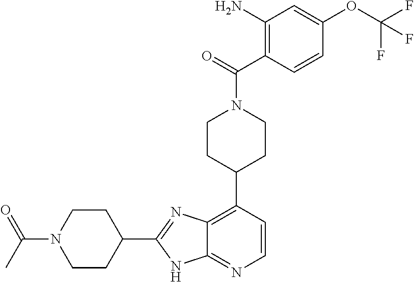 [SMILES: CC(=O)N1CCC(c2nc3c(C4CCN(C(=O)c5ccc(OC(F)(F)F)cc5N)CC4)ccnc3[nH]2)CC1] | 1-[4-[7-[1-[2-amino-4- (trifluoromethoxy)benzoyl]-4- piperidyl]-3H-imidazo[4,5-b]pyridin- 2-yl]-1-piperidyl]ethanone |
| 101 | 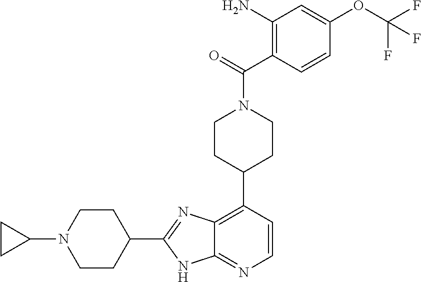 [SMILES: Nc1cc(OC(F)(F)F)ccc1C(=O)N1CCC(c2ccnc3[nH]c(C4CCN(C5CC5)CC4)nc23)CC1] | [2-amino-4- (trifluoromethoxy)phenyl]-[4-[2-(1- cyclopropyl-4-piperidyl)-3H- imidazo[4,5-b]pyridin-7-yl]-1- piperidyl]methanone |
| 102 | 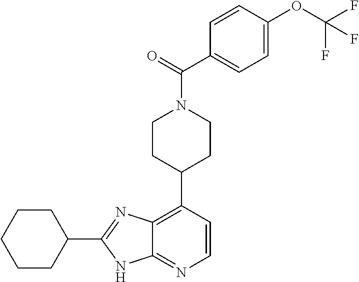 [SMILES: O=C(c1ccc(OC(F)(F)F)cc1)N1CCC(c2ccnc3[nH]c(C4CCCCC4)nc23)CC1] | [4-(2-cyclohexyl-3H-imidazo[4,5- b]pyridin-7-yl)-1-piperidyl]-[4- (trifluoromethoxy)phenyl]methanone |
| 103 | 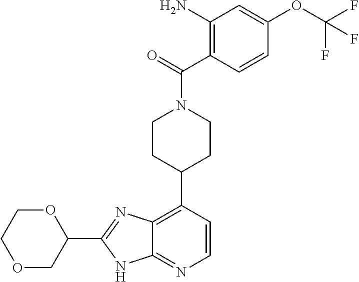 [SMILES: Nc1cc(OC(F)(F)F)ccc1C(=O)N1CCC(c2ccnc3[nH]c(C4COCCO4)nc23)CC1] | (rac)-[2-amino-4- (trifluoromethoxy)phenyl]-[4-[2-(1,4- dioxan-2-yl)-3H-imidazo[4,5- b]pyridin-7-yl]-1- piperidyl]methanone |
| 104 | 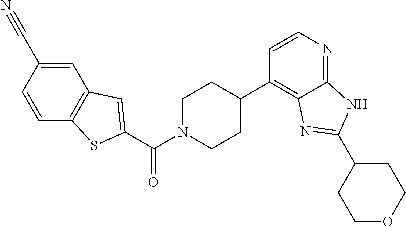 [SMILES: N#Cc1ccc2sc(C(=O)N3CCC(c4ccnc5[nH]c(C6CCOCC6)nc45)CC3)cc2c1] | 2-[4-(2-tetrahydropyran-4-yl-3H- imidazo[4,5-b]pyridin-7- yl)piperidine-1- carbonyl]benzothiophene-5- carbonitrile |
| 105 | 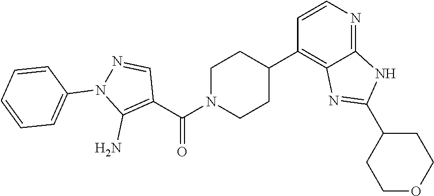 [SMILES: Nc1c(C(=O)N2CCC(c3ccnc4[nH]c(C5CCOCC5)nc34)CC2)cnn1-c1ccccc1] | (5-amino-1-phenyl-pyrazol-4-yl)-[4- (2-tetrahydropyran-4-yl-3H- imidazo[4,5-b]pyridin-7-yl)-1- piperidyl]methanone |
| 106 | 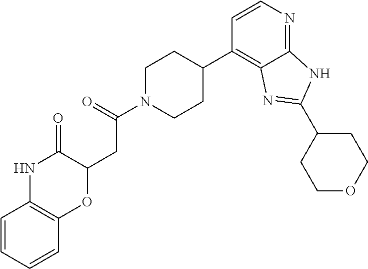 [SMILES: O=C1Nc2ccccc2OC1CC(=O)N1CCC(c2ccnc3[nH]c(C4CCOCC4)nc23)CC1] | 2-[2-oxo-2-[4-(2-tetrahydropyran-4- yl-3H-imidazo[4,5-b]pyridin-7-yl)-1- piperidyl]ethyl]-4H-1,4-benzoxazin- 3-one |
| 107 | 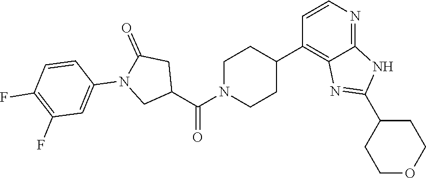 [SMILES: O=C(C1CC(=O)N(c2ccc(F)c(F)c2)C1)N1CCC(c2ccnc3[nH]c(C4CCOCC4)nc23)CC1] | 1-(3,4-difluorophenyl)-4-[4-(2- tetrahydropyran-4-yl-3H- imidazo[4,5-b]pyridin-7- yl)piperidine-1-carbonyl]pyrrolidin- 2-one |
| 108 | 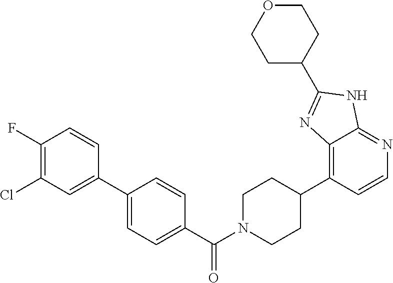 [SMILES: O=C(c1ccc(-c2ccc(F)c(Cl)c2)cc1)N1CCC(c2ccnc3[nH]c(C4CCOCC4)nc23)CC1] | [4-(3-chloro-4-fluoro- phenyl)phenyl]-[4-(2- tetrahydropyran-4-yl-3H- imidazo[4,5-b]pyridin-7-yl)-1- piperidyl]methanone |
| 109 | 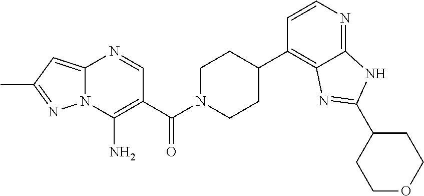 [SMILES: Cc1cc2ncc(C(=O)N3CCC(c4ccnc5[nH]c(C6CCOCC6)nc45)CC3)c(N)n2n1] | (7-amino-2-methyl-pyrazolo[1,5- a]pyrimidin-6-yl)-[4-(2- tetrahydropyran-4-yl-3H- imidazo[4,5-b]pyridin-7-yl)-1- piperidyl]methanone |
| 110 | 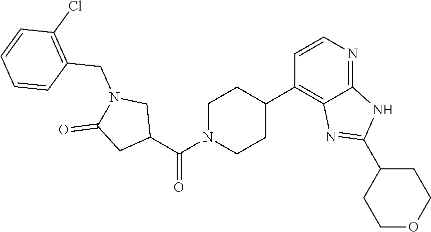 [SMILES: O=C1CC(C(=O)N2CCC(c3ccnc4[nH]c(C5CCOCC5)nc34)CC2)CN1Cc1ccccc1Cl] | 1-[(2-chlorophenyl)methyl]-4-[4-(2- tetrahydropyran-4-yl-3H- imidazo[4,5-b]pyridin-7- yl)piperidine-1-carbonyl]pyrrolidin- 2-one |
| 111 | 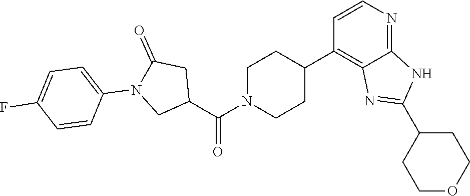 [SMILES: O=C(C1CC(=O)N(c2ccc(F)cc2)C1)N1CCC(c2ccnc3[nH]c(C4CCOCC4)nc23)CC1] | 1-(4-fluorophenyl)-4-[4-(2- tetrahydropyran-4-yl-3H- imidazo[4,5-b]pyridin-7- yl)piperidine-1-carbonyl]pyrrolidin- 2-one |
| 112 | 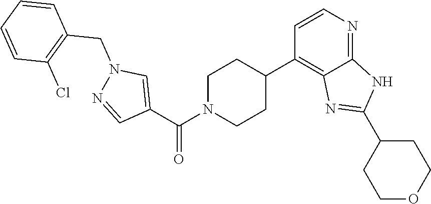 [SMILES: O=C(c1cnn(Cc2ccccc2Cl)c1)N1CCC(c2ccnc3[nH]c(C4CCOCC4)nc23)CC1] | [1-[(2-chlorophenyl)methyl]pyrazol- 4-yl]-[4-(2-tetrahydropyran-4-yl-3H- imidazo[4,5-b]pyridin-7-yl)-1- piperidyl]methanone |
| 113 | 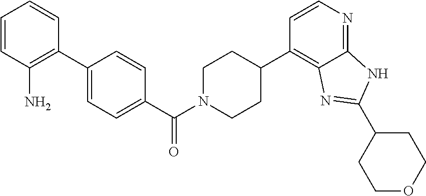 [SMILES: Nc1ccccc1-c1ccc(C(=O)N2CCC(c3ccnc4[nH]c(C5CCOCC5)nc34)CC2)cc1] | [4-(2-aminophenyl)phenyl]-[4-(2- tetrahydropyran-4-yl-3H- imidazo[4,5-b]pyridin-7-yl)-1- piperidyl]methanone |
| 114 | 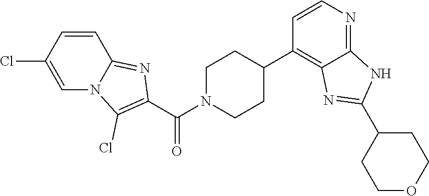 [SMILES: O=C(c1nc2ccc(Cl)cn2c1Cl)N1CCC(c2ccnc3[nH]c(C4CCOCC4)nc23)CC1] | (3,6-dichloroimidazo[1,2-a]pyridin- 2-yl)-[4-(2-tetrahydropyran-4-yl-3H- imidazo[4,5-b]pyridin-7-yl)-1- piperidyl]methanone |
| 115 | 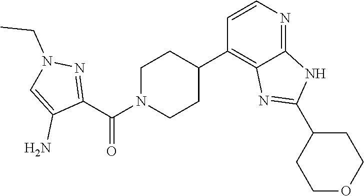 [SMILES: CCn1cc(N)c(C(=O)N2CCC(c3ccnc4[nH]c(C5CCOCC5)nc34)CC2)n1] | (4-amino-1-ethyl-pyrazol-3-yl)-[4-(2- tetrahydropyran-4-yl-3H- imidazo[4,5-b]pyridin-7-yl)-1- piperidyl]methanone |
| 116 | 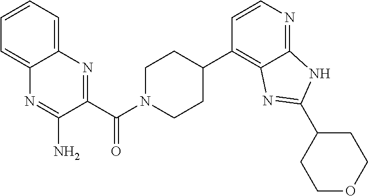 [SMILES: Nc1nc2ccccc2nc1C(=O)N1CCC(c2ccnc3[nH]c(C4CCOCC4)nc23)CC1] | (3-aminoquinoxalin-2-yl)-[4-(2- tetrahydropyran-4-yl-3H- imidazo[4,5-b]pyridin-7-yl)-1- piperidyl]methanone |
| 117 | 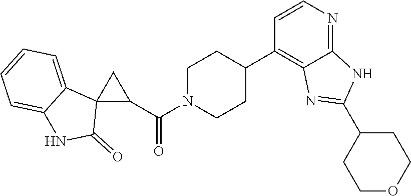 [SMILES: O=C(C1CC12C(=O)Nc1ccccc12)N1CCC(c2ccnc3[nH]c(C4CCOCC4)nc23)CC1] | 2-[4-(2-tetrahydropyran-4-yl-3H- imidazo[4,5-b]pyridin-7- yl)piperidine-1- carbonyl]spiro[cyclopropane-1,3′- indoline]-2′-one |
| 118 | 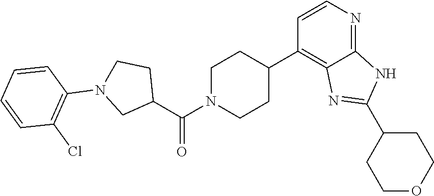 [SMILES: O=C(C1CCN(c2ccccc2Cl)C1)N1CCC(c2ccnc3[nH]c(C4CCOCC4)nc23)CC1] | [1-(2-chlorophenyl)pyrrolidin-3-yl]- [4-(2-tetrahydropyran-4-yl-3H- imidazo[4,5-b]pyridin-7-yl)-1- piperidyl]methanone |
| 119 | 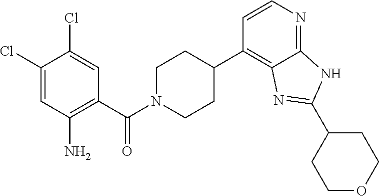 [SMILES: Nc1cc(Cl)c(Cl)cc1C(=O)N1CCC(c2ccnc3[nH]c(C4CCOCC4)nc23)CC1] | (2-amino-4,5-dichloro-phenyl)-[4-(2- tetrahydropyran-4-yl-3H- imidazo[4,5-b]pyridin-7-yl)-1- piperidyl]methanone |
| 120 | 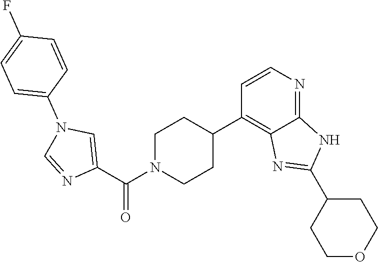 [SMILES: O=C(c1cn(-c2ccc(F)cc2)cn1)N1CCC(c2ccnc3[nH]c(C4CCOCC4)nc23)CC1] | [1-(4-fluorophenyl)imidazol-4-yl]-[4- (2-tetrahydropyran-4-yl-3H- imidazo[4,5-b]pyridin-7-yl)-1- piperidyl]methanone |
| 121 | 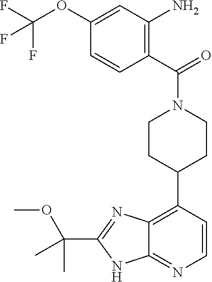 [SMILES: COC(C)(C)c1nc2c(C3CCN(C(=O)c4ccc(OC(F)(F)F)cc4N)CC3)ccnc2[nH]1] | [2-amino-4- (trifluoromethoxy)phenyl]-[4-[2-(1- methoxy-1-methyl-ethyl)-3H- imidazo[4,5-b]pyridin-7-yl]-1- piperidyl]methanone |
| 122 | 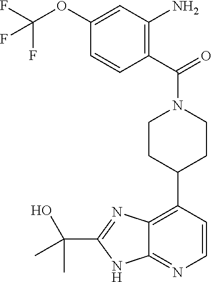 [SMILES: CC(C)(O)c1nc2c(C3CCN(C(=O)c4ccc(OC(F)(F)F)cc4N)CC3)ccnc2[nH]1] | [2-amino-4- (trifluoromethoxy)phenyl]-[4-[2-(1- hydroxy-1-methyl-ethyl)-3H- imidazo[4,5-b]pyridin-7-yl]-1- piperidyl]methanone |
| 123 | 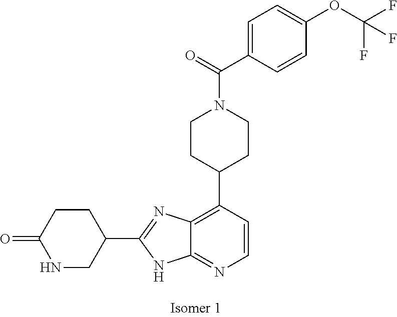 [SMILES: O=C1CCC(c2nc3c(C4CCN(C(=O)c5ccc(OC(F)(F)F)cc5)CC4)ccnc3[nH]2)CN1] | 5-[7-[1-[4- (trifluoromethoxy)benzoyl]-4- piperidyl]-3H-imidazo[4,5-b]pyridin- 2-yl]piperidin-2-one, Isomer 1 |
| 124 | 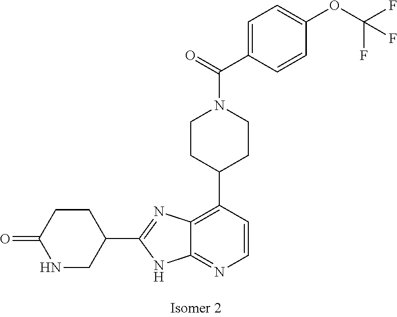 [SMILES: O=C1CCC(c2nc3c(C4CCN(C(=O)c5ccc(OC(F)(F)F)cc5)CC4)ccnc3[nH]2)CN1] | 5-[7-[1-[4- (trifluoromethoxy)benzoyl]-4- piperidyl]-3H-imidazo[4,5-b]pyridin- 2-yl]piperidin-2-one, Isomer 2 |
| 125 | 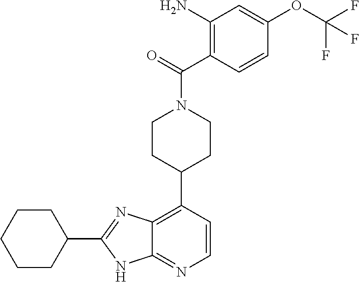 [SMILES: Nc1cc(OC(F)(F)F)ccc1C(=O)N1CCC(c2ccnc3[nH]c(C4CCCCC4)nc23)CC1] | [2-amino-4- (trifluoromethoxy)phenyl]-[4-(2- cyclohexyl-3H-imidazo[4,5- b]pyridin-7-yl)-1- piperidyl]methanone |
| 126 | 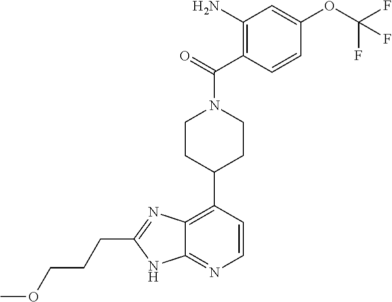 [SMILES: COCCCc1nc2c(C3CCN(C(=O)c4ccc(OC(F)(F)F)cc4N)CC3)ccnc2[nH]1] | [2-amino-4- (trifluoromethoxy)phenyl]-[4-[2-(3- methoxypropyl)-3H-imidazo[4,5- b]pyridin-7-yl]-1- piperidyl]methanone |
| 127 | 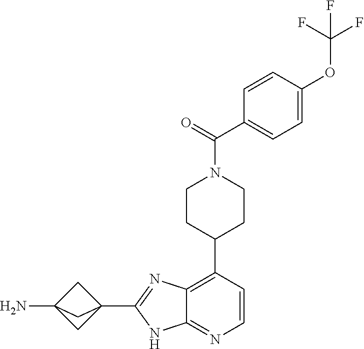 [SMILES: NC12CC(c3nc4c(C5CCN(C(=O)c6ccc(OC(F)(F)F)cc6)CC5)ccnc4[nH]3)(C1)C2] | [4-[2-(3-amino-1- bicyclo[1.1.1]pentanyl)-3H- imidazo[4,5-b]pyridin-7-yl]-1- piperidyl]-[4- (trifluoromethoxy)phenyl]methanone |
| 128 | 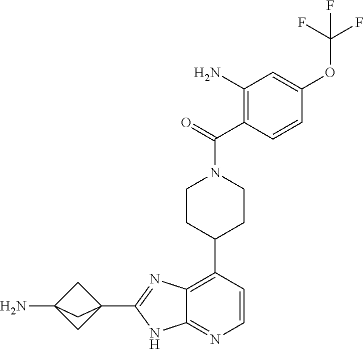 [SMILES: Nc1cc(OC(F)(F)F)ccc1C(=O)N1CCC(c2ccnc3[nH]c(C45CC(N)(C4)C5)nc23)CC1] | [4-[2-(3-amino-1- bicyclo[1.1.1]pentanyl)-3H- imidazo[4,5-b]pyridin-7-yl]-1- piperidyl]-[2-amino-4- (trifluoromethoxy)phenyl]methanone |
| 129 | 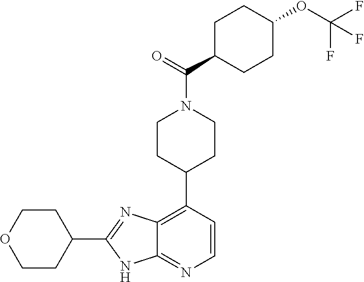 [SMILES: O=C([C@H]1CC[C@H](OC(F)(F)F)CC1)N1CCC(c2ccnc3[nH]c(C4CCOCC4)nc23)CC1] | (trans)-[4-(2-tetrahydropyran-4-yl- 3H-imidazo[4,5-b]pyridin-7-yl)-1- piperidyl]-[4- (trifluoromethoxy)cyclohexyl] methanone |
| 130 | 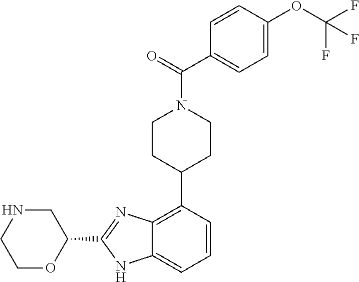 [SMILES: O=C(c1ccc(OC(F)(F)F)cc1)N1CCC(c2cccc3[nH]c([C@H]4CNCCO4)nc23)CC1] | [4-[2-[(2R)-morpholin-2-yl]-3H- imidazo[4,5-b]pyridin-7-yl]-1- piperidyl]-[4- (trifluoromethoxy)phenyl]methanone |
| 131 | 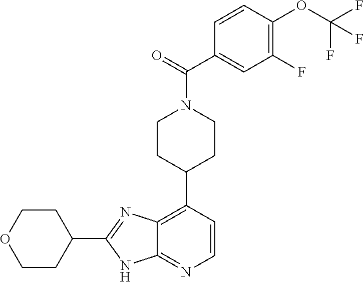 [SMILES: O=C(c1ccc(OC(F)(F)F)c(F)c1)N1CCC(c2ccnc3[nH]c(C4CCOCC4)nc23)CC1] | [3-fluoro-4- (trifluoromethoxy)phenyl]-[4-(2- tetrahydropyran-4-yl-3H- imidazo[4,5-b]pyridin-7-yl)-1- piperidyl]methanone |
| 132 | 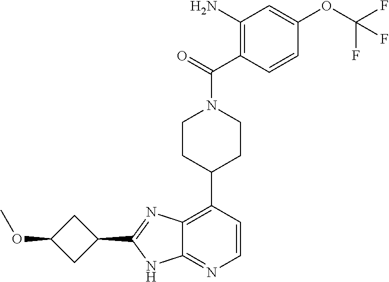 [SMILES: CO[C@H]1C[C@@H](c2nc3c(C4CCN(C(=O)c5ccc(OC(F)(F)F)cc5N)CC4)ccnc3[nH]2)C1] | (cis)-[2-amino-4- (trifluoromethoxy)phenyl]-[4-[2-(3- methoxycyclobutyl)-3H-imidazo[4,5- b]pyridin-7-yl]-1- piperidyl]methanone |
| 133 | 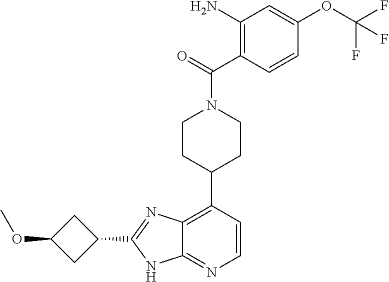 [SMILES: CO[C@H]1C[C@H](c2nc3c(C4CCN(C(=O)c5ccc(OC(F)(F)F)cc5N)CC4)ccnc3[nH]2)C1] | (trans)-[2-amino-4- (trifluoromethoxy)phenyl]-[4-[2-(3- methoxycyclobutyl)-3H-imidazo[4,5- b]pyridin-7-yl]-1- piperidyl]methanone |
| 134 | 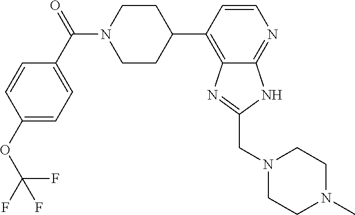 [SMILES: CN1CCN(Cc2nc3c(C4CCN(C(=O)c5ccc(OC(F)(F)F)cc5)CC4)ccnc3[nH]2)CC1] 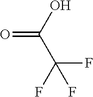 | [4-[2-[(4-methylpiperazin-1- yl)methyl]-3H-imidazo[4,5- b]pyridin-7-yl]-1-piperidyl]-[4- (trifluoromethoxy)phenyl]methanone; 2,2,2-trifluoroacetic acid |
| 135 | 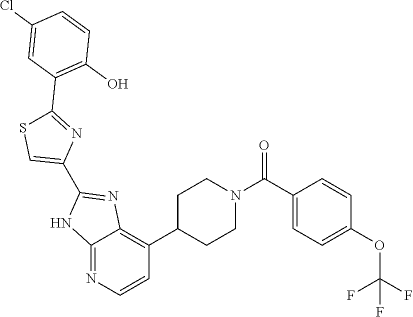 [SMILES: O=C(c1ccc(OC(F)(F)F)cc1)N1CCC(c2ccnc3[nH]c(-c4csc(-c5cc(Cl)ccc5O)n4)nc23)CC1] | [4-[2-[2-(5-chloro-2-hydroxy- phenyl)thiazol-4-yl]-3H-imidazo[4,5- b]pyridin-7-yl]-1-piperidyl]-[4- (trifluoromethoxy)phenyl]methanone |
| 136 | 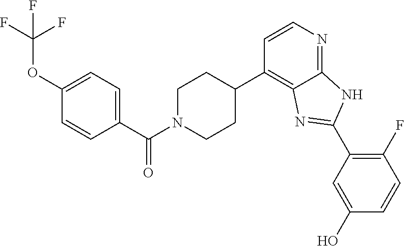 [SMILES: O=C(c1ccc(OC(F)(F)F)cc1)N1CCC(c2ccnc3[nH]c(-c4cc(O)ccc4F)nc23)CC1] | [4-[2-(2-fluoro-5-hydroxy-phenyl)- 3H-imidazo[4,5-b]pyridin-7-yl]-1- piperidyl]-[4- (trifluoromethoxy)phenyl]methanone |
| 137 | 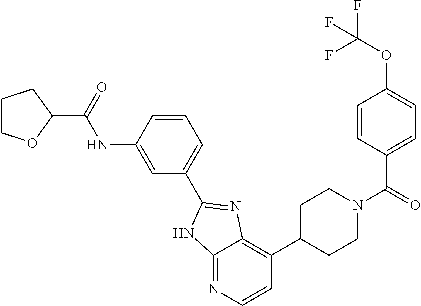 [SMILES: O=C(Nc1cccc(-c2nc3c(C4CCN(C(=O)c5ccc(OC(F)(F)F)cc5)CC4)ccnc3[nH]2)c1)C1CCCO1] | N-[3-[7-[1-[4- (trifluoromethoxy)benzoyl]-4- piperidyl]-3H-imidazo[4,5-b]pyridin- 2-yl]phenyl]tetrahydrofuran-2- carboxamide |
| 138 | 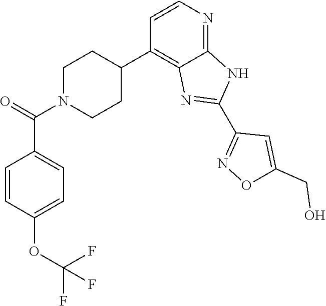 [SMILES: O=C(c1ccc(OC(F)(F)F)cc1)N1CCC(c2ccnc3[nH]c(-c4cc(CO)on4)nc23)CC1] | [4-[2-[5-(hydroxymethyl)isoxazol-3- yl]-3H-imidazo[4,5-b]pyridin-7-yl]- 1-piperidyl]-[4- (trifluoromethoxy)phenyl]methanone |
| 139 | 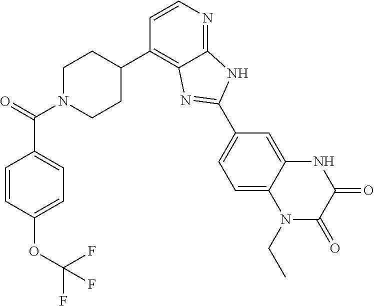 [SMILES: CCn1c(=O)c(=O)[nH]c2cc(-c3nc4c(C5CCN(C(=O)c6ccc(OC(F)(F)F)cc6)CC5)ccnc4[nH]3)ccc21] | 1-ethyl-3-hydroxy-6-[7-[1-[4- (trifluoromethoxy)benzoyl]-4- piperidyl]-3H-imidazo[4,5-b]pyridin- 2-yl]quinoxalin-2-one |
| 140 | 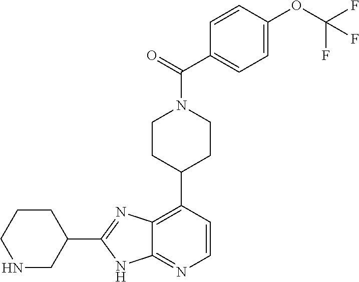 [SMILES: O=C(c1ccc(OC(F)(F)F)cc1)N1CCC(c2ccnc3[nH]c(C4CCCNC4)nc23)CC1] | (rac)-[4-[2-(3-piperidyl)-3H- imidazo[4,5-b]pyridin-7-yl]-1- piperidyl]-[4- (trifluoromethoxy)phenyl]methanone |
| 141 | 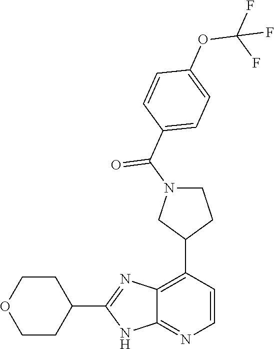 [SMILES: O=C(c1ccc(OC(F)(F)F)cc1)N1CCC(c2ccnc3[nH]c(C4CCOCC4)nc23)C1] | (rac)-[3-(2-tetrahydropyran-4-yl-3H- imidazo[4,5-b]pyridin-7- yl)pyrrolidin-1-yl]-[4- (trifluoromethoxy)phenyl]methanone |
| 142 | 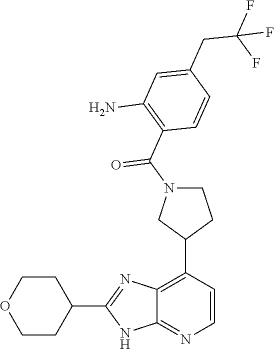 [SMILES: Nc1cc(CC(F)(F)F)ccc1C(=O)N1CCC(c2ccnc3[nH]c(C4CCOCC4)nc23)C1] | (rac)-[2-amino-4- (trifluoromethoxy)phenyl]-[3-(2- tetrahydropyran-4-yl-3H- imidazo[4,5-b]pyridin-7- yl)pyrrolidin-1-yl]methanone |
| 143 | 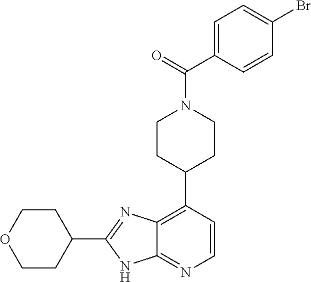 [SMILES: O=C(c1ccc(Br)cc1)N1CCC(c2ccnc3[nH]c(C4CCOCC4)nc23)CC1] | (4-bromophenyl)-[4-(2- tetrahydropyran-4-yl-3H- imidazo[4,5-b]pyridin-7-yl)-1- piperidyl]methanone |
| 144 | 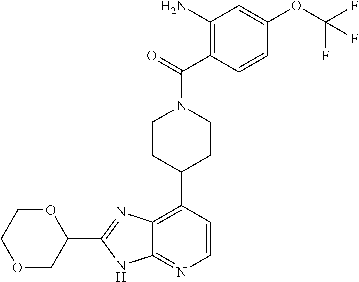 [SMILES: Nc1cc(OC(F)(F)F)ccc1C(=O)N1CCC(c2ccnc3[nH]c(C4COCCO4)nc23)CC1] Isomer 1 | [2-amino-4- (trifluoromethoxy)phenyl]-[4-[2-[1,4- dioxan-2-yl]-3H-imidazo[4,5- b]pyridin-7-yl]-1- piperidyl]methanone, Isomer 1 |
| 145 | 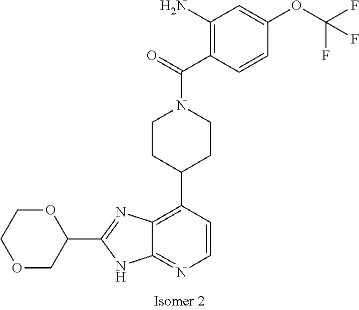 [SMILES: Nc1cc(OC(F)(F)F)ccc1C(=O)N1CCC(c2ccnc3[nH]c(C4COCCO4)nc23)CC1] | [2-amino-4- (trifluoromethoxy)phenyl]-[4-[2-[1,4- dioxan-2-yl]-3H-imidazo[4,5- b]pyridin-7-yl]-1- piperidyl]methanone, Isomer 2 |
| 146 | 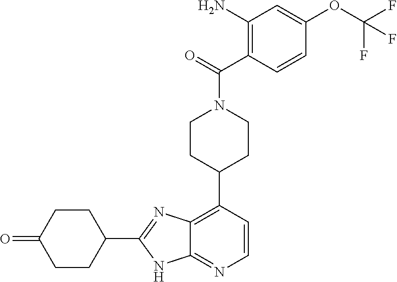 [SMILES: Nc1cc(OC(F)(F)F)ccc1C(=O)N1CCC(c2ccnc3[nH]c(C4CCC(=O)CC4)nc23)CC1] | 4-[7-[1-[2-amino-4- (trifluoromethoxy)benzoyl]-4- piperidyl]-3H-imidazo[4,5-b]pyridin- 2-yl]cyclohexanone |
| 147 | 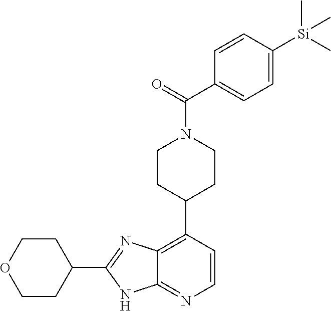 [SMILES: C[Si](C)(C)c1ccc(C(=O)N2CCC(c3ccnc4[nH]c(C5CCOCC5)nc34)CC2)cc1] | [4-(2-tetrahydropyran-4-yl-3H- imidazo[4,5-b]pyridin-7-yl)-1- piperidyl]-(4- trimethylsilylphenyl)methanone |
| 148 | 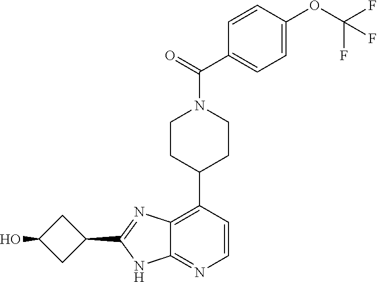 [SMILES: O=C(c1ccc(OC(F)(F)F)cc1)N1CCC(c2ccnc3[nH]c([C@H]4C[C@@H](O)C4)nc23)CC1] | (cis)-[4-[2-(3-hydroxycyclobutyl)- 3H-imidazo[4,5-b]pyridin-7-yl]-1- piperidyl]-[4- (trifluoromethoxy)phenyl]methanone |
| 149 | 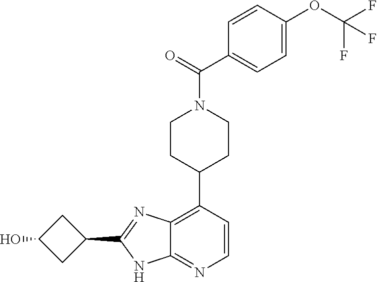 [SMILES: O=C(c1ccc(OC(F)(F)F)cc1)N1CCC(c2ccnc3[nH]c([C@H]4C[C@H](O)C4)nc23)CC1] | (trans)-[4-[2-(3-hydroxycyclobutyl)- 3H-imidazo[4,5-b]pyridin-7-yl]-1- piperidyl]-[4- (trifluoromethoxy)phenyl]methanone |
| 150 | 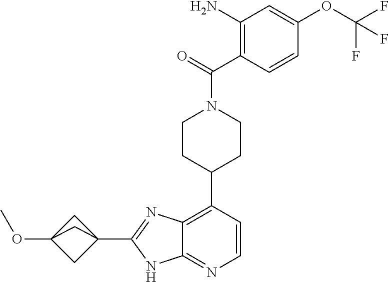 [SMILES: COC12CC(c3nc4c(C5CCN(C(=O)c6ccc(OC(F)(F)F)cc6N)CC5)ccnc4[nH]3)(C1)C2] | [2-amino-4- (trifluoromethoxy)phenyl]-[4-[2-(3- methoxy-1-bicyclo[1.1.1]pentanyl)- 3H-imidazo[4,5-b]pyridin-7-yl]-1- piperidyl]methanone |
| 151 | 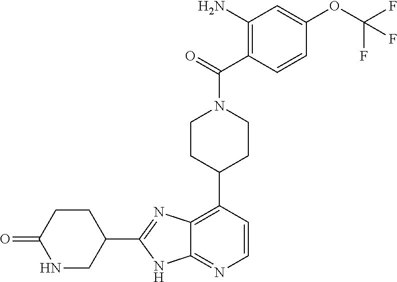 [SMILES: Nc1cc(OC(F)(F)F)ccc1C(=O)N1CCC(c2ccnc3[nH]c(C4CCC(=O)NC4)nc23)CC1] Isomer 1 | 5-[7-[1-[2-amino-4- (trifluoromethoxy)benzoyl]-4- piperidyl]-3H-imidazo[4,5-b]pyridin- 2-yl]piperidin-2-one, Isomer 1 |
| 152 | 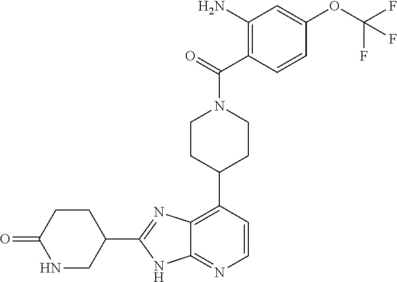 [SMILES: Nc1cc(OC(F)(F)F)ccc1C(=O)N1CCC(c2ccnc3[nH]c(C4CCC(=O)NC4)nc23)CC1] Isomer 2 | 5-[7-[1-[2-amino-4- (trifluoromethoxy)benzoyl]-4- piperidyl]-3H-imidazo[4,5-b]pyridin- 2-yl]piperidin-2-one, Isomer 2 |
| 153 | 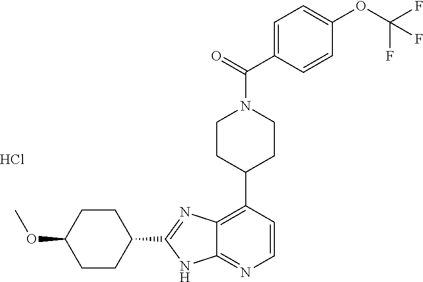 [SMILES: Cl ; CO[C@H]1CC[C@H](c2nc3c(C4CCN(C(=O)c5ccc(OC(F)(F)F)cc5)CC4)ccnc3[nH]2)CC1] | (trans)-[4-(trifluoromethoxy)phenyl]- [4-[2-(4-methoxycyclohexyl)-3H- imidazo[4,5-b]pyridin-7-yl]-1- piperidyl]methanone;hydrochloride |
| 154 | 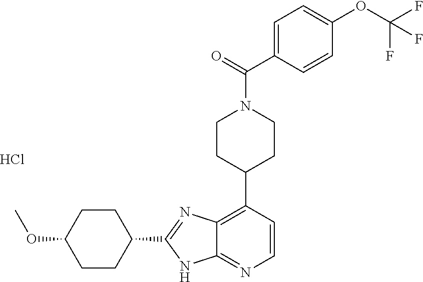 [SMILES: Cl ; CO[C@H]1CC[C@@H](c2nc3c(C4CCN(C(=O)c5ccc(OC(F)(F)F)cc5)CC4)ccnc3[nH]2)CC1] | (cis)-[4-(trifluoromethoxy)phenyl]- [4-[2-(4-methoxycyclohexyl)-3H- imidazo[4,5-b]pyridin-7-yl]-1- piperidyl]methanone;hydrochloride |
| 155 | 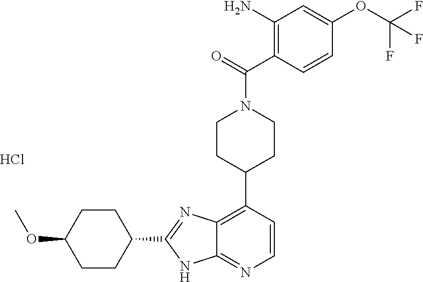 [SMILES: Cl ; CO[C@H]1CC[C@H](c2nc3c(C4CCN(C(=O)c5ccc(OC(F)(F)F)cc5N)CC4)ccnc3[nH]2)CC1] | (trans)-[2-amino-4- (trifluoromethoxy)phenyl]-[4-[2-(4- methoxycyclohexyl)-3H- imidazo[4,5-b]pyridin-7-yl]-1- piperidyl]methanone;hydrochloride |
| 156 | 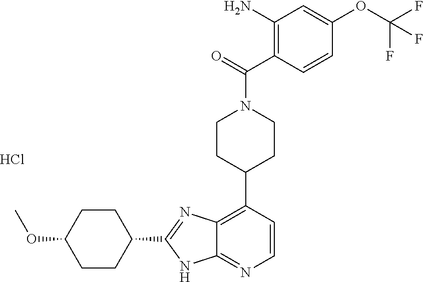 [SMILES: Cl ; CO[C@H]1CC[C@@H](c2nc3c(C4CCN(C(=O)c5ccc(OC(F)(F)F)cc5N)CC4)ccnc3[nH]2)CC1] | (cis)-[2-amino-4- (trifluoromethoxy)phenyl]-[4-[2-(4- methoxycyclohexyl)-3H- imidazo[4,5-b]pyridin-7-yl]-1- piperidyl]methanone;hydrochloride |
| 157 | 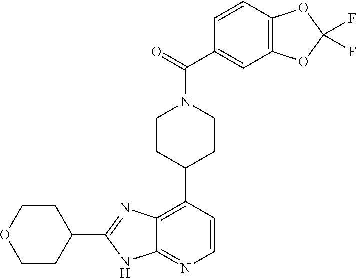 [SMILES: O=C(c1ccc2c(c1)OC(F)(F)O2)N1CCC(c2ccnc3[nH]c(C4CCOCC4)nc23)CC1] | (2,2-difluoro-1,3-benzodioxol-5-yl)- [4-(2-tetrahydropyran-4-yl-3H- imidazo[4,5-b]pyridin-7-yl)-1- piperidyl]methanone |
| 158 | 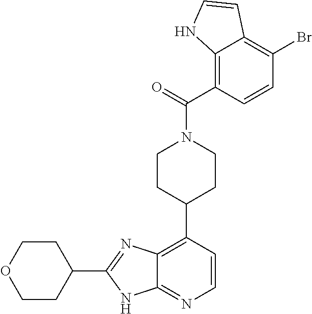 [SMILES: O=C(c1ccc(Br)c2cc[nH]c12)N1CCC(c2ccnc3[nH]c(C4CCOCC4)nc23)CC1] | (4-bromo-1H-indol-7-yl)-[4-(2- tetrahydropyran-4-yl-3H- imidazo[4,5-b]pyridin-7-yl)-1- piperidyl]methanone |
| 159 | 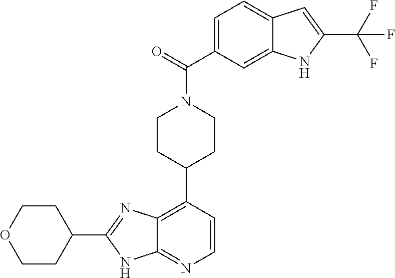 [SMILES: O=C(c1ccc2cc(C(F)(F)F)[nH]c2c1)N1CCC(c2ccnc3[nH]c(C4CCOCC4)nc23)CC1] | [4-(2-tetrahydropyran-4-yl-3H- imidazo[4,5-b]pyridin-7-yl)-1- piperidyl]-[2-(trifluoromethyl)-1H- indol-6-yl]methanone |
| 160 | 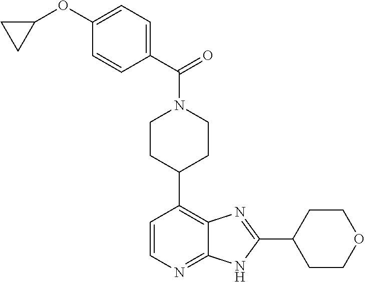 [SMILES: O=C(c1ccc(OC2CC2)cc1)N1CCC(c2ccnc3[nH]c(C4CCOCC4)nc23)CC1] | [4-(cyclopropoxy)phenyl]-[4-(2- tetrahydropyran-4-yl-3H- imidazo[4,5-b]pyridin-7-yl)-1- piperidyl]methanone |
| 161 | 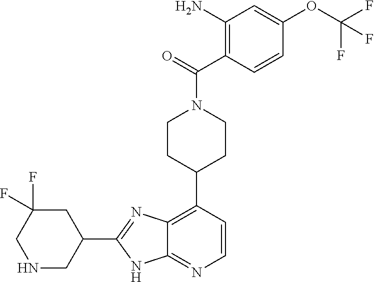 [SMILES: Nc1cc(OC(F)(F)F)ccc1C(=O)N1CCC(c2ccnc3[nH]c(C4CNCC(F)(F)C4)nc23)CC1] Isomer 1 | [2-amino-4- (trifluoromethoxy)phenyl]-[4-[2-[5,5- difluoro-3-piperidyl]-3H- imidazo[4,5-b]pyridin-7-yl]-1- piperidyl]methanone, Isomer 1 |
| 162 | 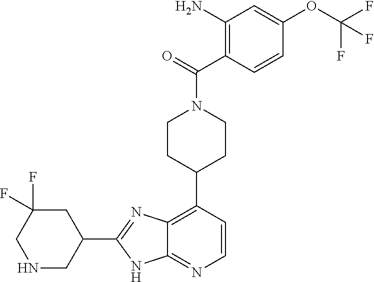 [SMILES: Nc1cc(OC(F)(F)F)ccc1C(=O)N1CCC(c2ccnc3[nH]c(C4CNCC(F)(F)C4)nc23)CC1] Isomer 2 | [2-amino-4- (trifluoromethoxy)phenyl]-[4-[2-[5,5- difluoro-3-piperidyl]-3H- imidazo[4,5-b]pyridin-7-yl]-1- piperidyl]methanone, Isomer 2 |
| 163 | 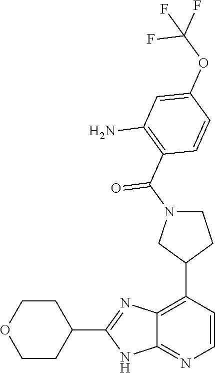 [SMILES: Nc1cc(OC(F)(F)F)ccc1C(=O)N1CCC(c2ccnc3[nH]c(C4CCOCC4)nc23)C1] | [2-amino-4- (trifluoromethoxy)phenyl]-[3-(2- tetrahydropyran-4-yl-3H- imidazo[4,5-b]pyridin-7- yl)pyrrolidin-1-yl]methanone, Isomer 1 |
| 164 | 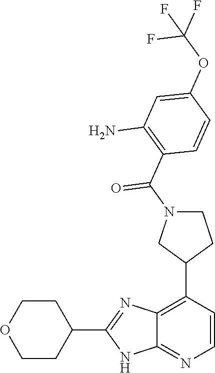 [SMILES: Nc1cc(OC(F)(F)F)ccc1C(=O)N1CCC(c2ccnc3[nH]c(C4CCOCC4)nc23)C1] | [2-amino-4- (trifluoromethoxy)phenyl]-[3-(2- tetrahydropyran-4-yl-3H- imidazo[4,5-b]pyridin-7- yl)pyrrolidin-1-yl]methanone, Isomer 2 |
| 165 | 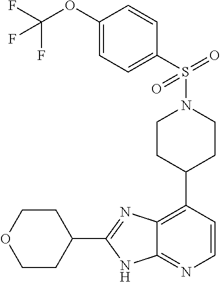 [SMILES: O=S(=O)(c1ccc(OC(F)(F)F)cc1)N1CCC(c2ccnc3[nH]c(C4CCOCC4)nc23)CC1] | 2-tetrahydropyran-4-yl-7-[1-[4- (trifluoromethoxy)phenyl]sulfonyl-4- piperidyl]-3H-imidazo[4,5-b]pyridine |
| 166 | 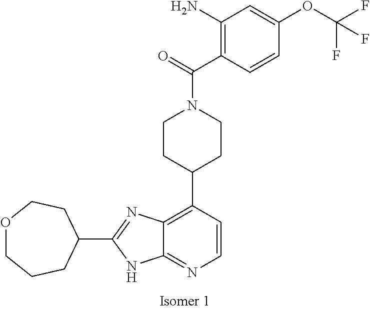 [SMILES: Nc1cc(OC(F)(F)F)ccc1C(=O)N1CCC(c2ccnc3[nH]c(C4CCCOCC4)nc23)CC1] | [2-amino-4- (trifluoromethoxy)phenyl]-[4-[2- [oxepan-4-yl]-3H-imidazo[4,5- b]pyridin-7-yl]-1- piperidyl]methanone, Isomer 1 |
| 167 | 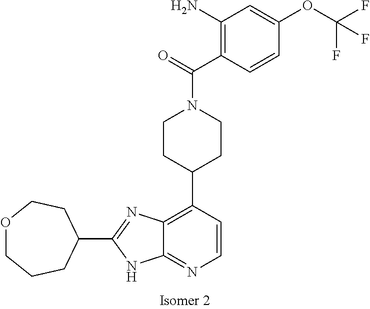 [SMILES: Nc1cc(OC(F)(F)F)ccc1C(=O)N1CCC(c2ccnc3[nH]c(C4CCCOCC4)nc23)CC1] | [2-amino-4- (trifluoromethoxy)phenyl]-[4-[2- [oxepan-4-yl]-3H-imidazo[4,5- b]pyridin-7-yl]-1- piperidyl]methanone, Isomer 2 |
| 168 | 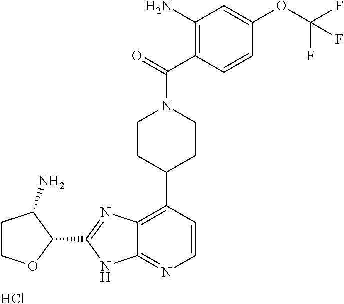 [SMILES: Nc1cc(OC(F)(F)F)ccc1C(=O)N1CCC(c2ccnc3[nH]c([C@@H]4OCC[C@@H]4N)nc23)CC1 ; Cl] | [4-[2-[rac-(2R,3S)-3- aminotetrahydrofuran-2-yl]-3H- imidazo[4,5-b]pyridin-7-yl]-1- piperidyl]-[4- (trifluoromethoxy)phenyl]methanone; hydrochloride |
| 169 | 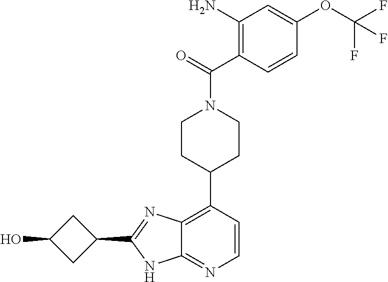 [SMILES: Nc1cc(OC(F)(F)F)ccc1C(=O)N1CCC(c2ccnc3[nH]c([C@H]4C[C@@H](O)C4)nc23)CC1] | (cis)-[2-amino-4- (trifluoromethoxy)phenyl]-[4-[2-(3- hydroxycyclobutyl)-3H-imidazo[4,5- b]pyridin-7-yl]-1- piperidyl]methanone |
| 170 | 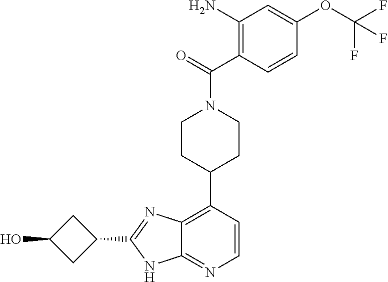 [SMILES: Nc1cc(OC(F)(F)F)ccc1C(=O)N1CCC(c2ccnc3[nH]c([C@H]4C[C@H](O)C4)nc23)CC1] | (trans)-[2-amino-4- (trifluoromethoxy)phenyl]-[4-[2-(3- hydroxycyclobutyl)-3H-imidazo[4,5- b]pyridin-7-yl]-1- piperidyl]methanone |
| 171 | 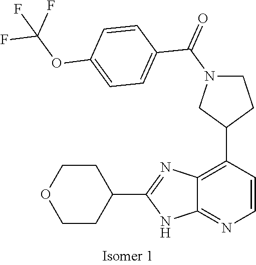 [SMILES: O=C(c1ccc(OC(F)(F)F)cc1)N1CCC(c2ccnc3[nH]c(C4CCOCC4)nc23)C1] | [3-(2-tetrahydropyran-4-yl-3H- imidazo[4,5-b]pyridin-7- yl)pyrrolidin-1-yl]-[4- (trifluoromethoxy)phenyl]methanone, Isomer 1 |
| 172 | 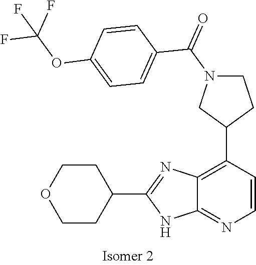 [SMILES: O=C(c1ccc(OC(F)(F)F)cc1)N1CCC(c2ccnc3[nH]c(C4CCOCC4)nc23)C1] | [3-(2-tetrahydropyran-4-yl-3H- imidazo[4,5-b]pyridin-7- yl)pyrrolidin-1-yl]-[4- (trifluoromethoxy)phenyl]methanone, Isomer 2 |
| 173 | 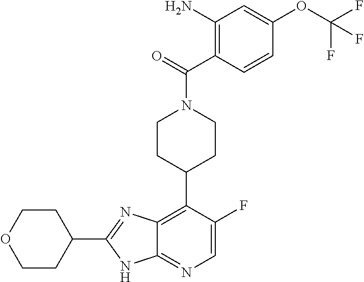 [SMILES: Nc1cc(OC(F)(F)F)ccc1C(=O)N1CCC(c2c(F)cnc3[nH]c(C4CCOCC4)nc23)CC1] | [2-amino-4- (trifluoromethoxy)phenyl]-[4-(6- fluoro-2-tetrahydropyran-4-yl-3H- imidazo[4,5-b]pyridin-7-yl)-1- piperidyl]methanone |
| 174 | 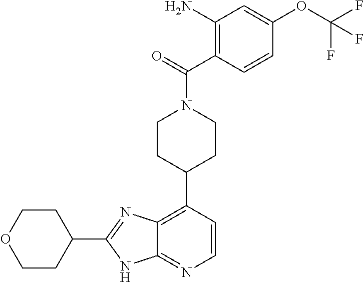 [SMILES: Nc1cc(OC(F)(F)F)ccc1C(=O)N1CCC(c2ccnc3[nH]c(C4CCOCC4)nc23)CC1] | [2-amino-4- (trifluoromethoxy)phenyl]-[4-(2- morpholino-3H-imidazo[4,5- b]pyridin-7-yl)-1- piperidyl]methanone |
| 175 | 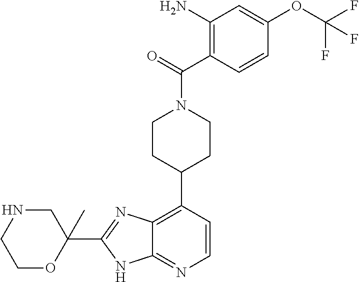 [SMILES: CC1(c2nc3c(C4CCN(C(=O)c5ccc(OC(F)(F)F)cc5N)CC4)ccnc3[nH]2)CNCCO1] | (rac)-[2-amino-4- (trifluoromethoxy)phenyl]-[4-[2-(2- methylmorpholin-2-yl)-3H- imidazo[4,5-b]pyridin-7-yl]-1- piperidyl]methanone |
| 176 | 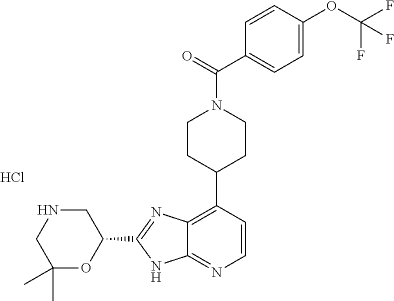 [SMILES: Cl ; CC1(C)CNC[C@H](c2nc3c(C4CCN(C(=O)c5ccc(OC(F)(F)F)cc5)CC4)ccnc3[nH]2)O1] | [4-[2-[(2S)-6,6-dimethylmorpholin- 2-yl]-3H-imidazo[4,5-b]pyridin-7- yl]-1-piperidyl]-[4- (trifluoromethoxy)phenyl]methanone; hydrochloride |
| 177 | 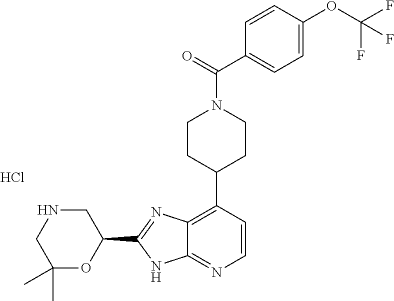 [SMILES: Cl ; CC1(C)CNC[C@@H](c2nc3c(C4CCN(C(=O)c5ccc(OC(F)(F)F)cc5)CC4)ccnc3[nH]2)O1] | [4-[2-[(2R)-6,6-dimethylmorpholin- 2-yl]-3H-imidazo[4,5-b]pyridin-7- yl]-1-piperidyl]-[4- (trifluoromethoxy)phenyl]methanone; hydrochloride |
| 178 | 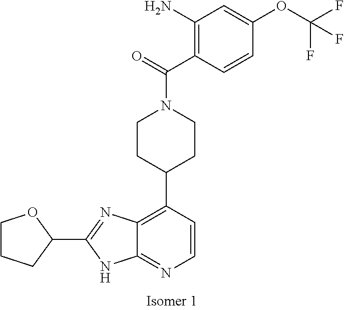 [SMILES: Nc1cc(OC(F)(F)F)ccc1C(=O)N1CCC(c2ccnc3[nH]c(C4CCCO4)nc23)CC1] | [2-amino-4- (trifluoromethoxy)phenyl]-[4-[2- [tetrahydrofuran-2-yl]-3H- imidazo[4,5-b]pyridin-7-y]]-1- piperidyl]methanone, Isomer 1 |
| 179 | 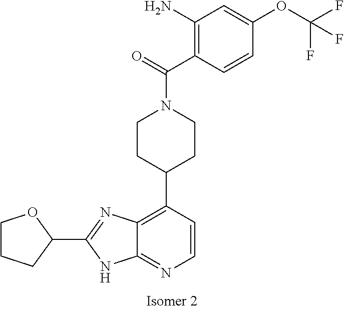 [SMILES: Nc1cc(OC(F)(F)F)ccc1C(=O)N1CCC(c2ccnc3[nH]c(C4CCCO4)nc23)CC1] | [2-amino-4- (trifluoromethoxy)phenyl]-[4-[2- [tetrahydrofuran-2-yl]-3H- imidazo[4,5-b]pyridin-7-yl]-1- piperidyl]methanone, Isomer 2 |
| 180 | 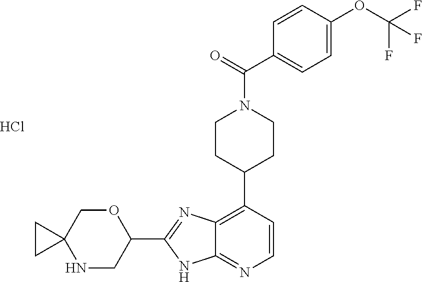 [SMILES: Cl ; O=C(c1ccc(OC(F)(F)F)cc1)N1CCC(c2ccnc3[nH]c(C4CNC5(CC5)CO4)nc23)CC1] | (rac)-[4-[2-(7-oxa-4- azaspiro[2.5]octan-6-yl)-3H- imidazo[4,5-b]pyridin-7-yl]-1- piperidyl]-[4- (trifluoromethoxy)phenyl]methanone; hydrochloride |
| 181 | 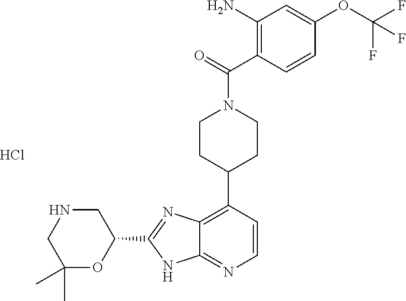 [SMILES: Cl ; CC1(C)CNC[C@H](c2nc3c(C4CCN(C(=O)c5ccc(OC(F)(F)F)cc5N)CC4)ccnc3[nH]2)O1] | [2-amino-4- (trifluoromethoxy)phenyl]-[4-[2- [(2S)-6,6-dimethylmorpholin-2-yl]- 3H-imidazo[4,5-b]pyridin-7-yl]-1- piperidyl]methanone;hydrochloride |
| 182 | 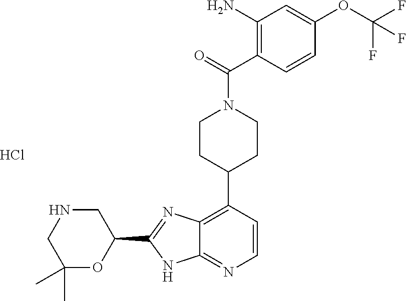 [SMILES: Cl ; CC1(C)CNC[C@@H](c2nc3c(C4CCN(C(=O)c5ccc(OC(F)(F)F)cc5N)CC4)ccnc3[nH]2)O1] | [2-amino-4- (trifluoromethoxy)phenyl]-[4-[2- [(2R)-6,6-dimethylmorpholin-2-yl]- 3H-imidazo[4,5-b]pyridin-7-yl]-1- piperidyl]methanone;hydrochloride |
| 183 | 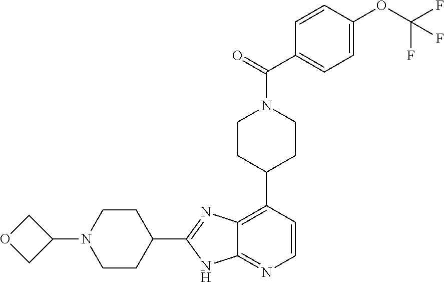 [SMILES: O=C(c1ccc(OC(F)(F)F)cc1)N1CCC(c2ccnc3[nH]c(C4CCN(C5COC5)CC4)nc23)CC1] | [4-[2-[1-(oxetan-3-yl)-4-piperidyl]- 3H-imidazo[4,5-b]pyridin-7-yl]-1- piperidyl]-[4- (trifluoromethoxy)phenyl]methanone |
| 184 | 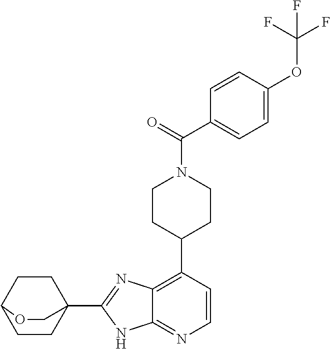 [SMILES: O=C(c1ccc(OC(F)(F)F)cc1)N1CCC(c2ccnc3[nH]c(C45CCC(CC4)OC5)nc23)CC1] | [4-[2-(2-oxabicyclo[2.2.2]octan-4- yl)-3H-imidazo[4,5-b]pyridin-7-yl]- 1-piperidyl]-[4- (trifluoromethoxy)phenyl]methanone |
| 185 | 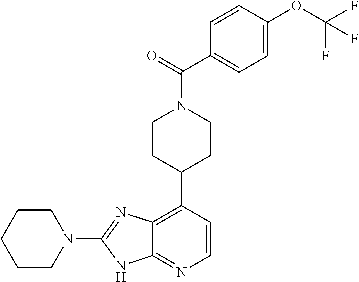 [SMILES: O=C(c1ccc(OC(F)(F)F)cc1)N1CCC(c2ccnc3[nH]c(N4CCCCC4)nc23)CC1] | [4-(2-morpholino-3H-imidazo[4,5- b]pyridin-7-yl)-1-piperidyl]-[4- (trifluoromethoxy)phenyl]methanone |
| 186 | 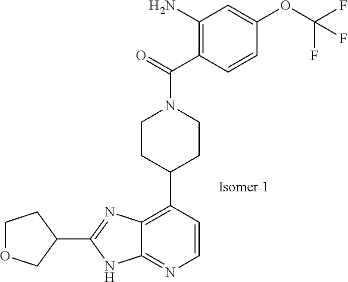 [SMILES: Nc1cc(OC(F)(F)F)ccc1C(=O)N1CCC(c2ccnc3[nH]c(C4CCOC4)nc23)CC1] | [2-amino-4- (trifluoromethoxy)phenyl]-[4-[2- [tetrahydrofuran-3-yl]-3H- imidazo[4,5-b]pyridin-7-yl]-1- piperidyl]methanone, Isomer 1 |
| 187 | 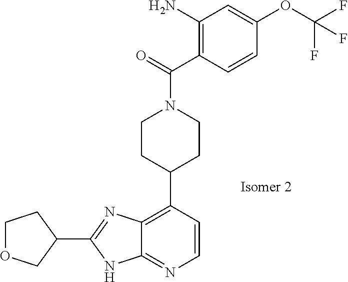 [SMILES: Nc1cc(OC(F)(F)F)ccc1C(=O)N1CCC(c2ccnc3[nH]c(C4CCOC4)nc23)CC1] | [2-amino-4- (trifluoromethoxy)phenyl]-[4-[2- [tetrahydrofuran-3-yl]-3H- imidazo[4,5-b]pyridin-7-yl]-1- piperidyl]methanone, Isomer 2 |
| 188 | 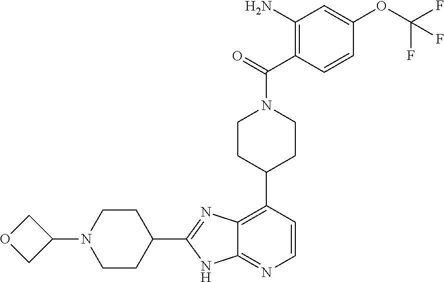 [SMILES: Nc1cc(OC(F)(F)F)ccc1C(=O)N1CCC(c2ccnc3[nH]c(C4CCN(C5COC5)CC4)nc23)CC1] | [2-amino-4- trifluoromethoxy)phenyl]-[4-[2-[1- (oxetan-3-yl)-4-piperidyl]-3H- imidazo[4,5-b]pyridin-7-yl]-1- piperidyl]methanone |
| 189 | 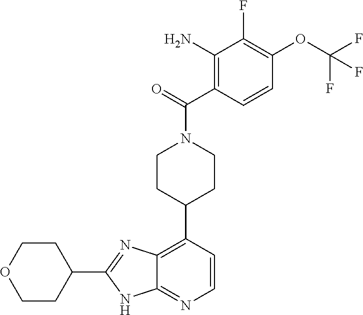 [SMILES: Nc1c(C(=O)N2CCC(c3ccnc4[nH]c(C5CCOCC5)nc34)CC2)ccc(OC(F)(F)F)c1F] | [2-amino-3-fluoro-4- (trifluoromethoxy)phenyl]-[4-(2- tetrahydropyran-4-yl-3H- imidazo[4,5-b]pyridin-7-yl)-1- piperidyl]methanone |
| 190 | 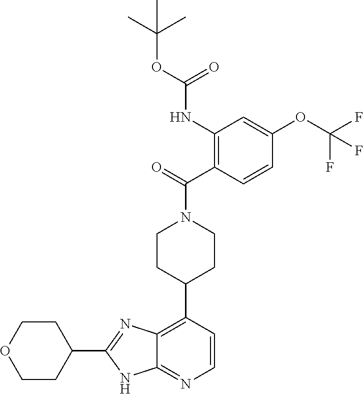 [SMILES: CC(C)(C)OC(=O)Nc1cc(OC(F)(F)F)ccc1C(=O)N1CCC(c2ccnc3[nH]c(C4CCOCC4)nc23)CC1] | tert-butyl N-[2-[4-(2- tetrahydropyran-4-yl-3H- imidazo[4,5-b]pyridin-7- yl)piperidine-1-carbonyl]-5- (trifluoromethoxy)phenyl]carbamate |
| 191 | 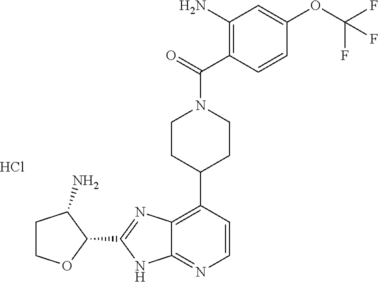 [SMILES: Cl ; Nc1cc(OC(F)(F)F)ccc1C(=O)N1CCC(c2ccnc3[nH]c([C@@H]4OCC[C@@H]4N)nc23)CC1] | [4-[2-[rac-(2R,3S)-3- aminotetrahydrofuran-2-yl]-3H- imidazo[4,5-b]pyridin-7-yl]-1- piperidyl]-[2-amino-4- (trifluoromethoxy)phenyl]methanone; hydrochloride |
| 192 | 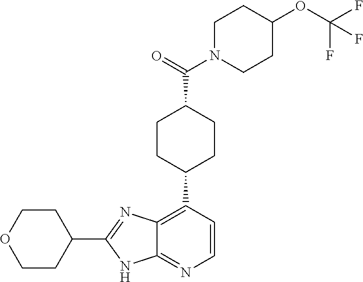 [SMILES: O=C([C@H]1CC[C@@H](c2ccnc3[nH]c(C4CCOCC4)nc32)CC1)N1CCC(OC(F)(F)F)CC1] | (cis)-[4-(2-tetrahydropyran-4-yl-3H- imidazo[4,5-b]pyridin-7- yl)cyclohexyl]-[4-(trifluoromethoxy)- 1-piperidyl]methanone |
| 193 | 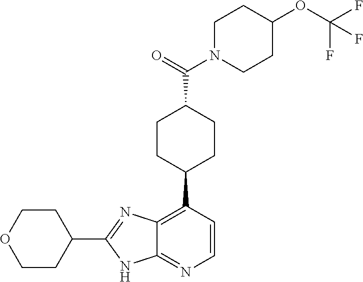 [SMILES: O=C([C@H]1CC[C@H](c2ccnc3[nH]c(C4CCOCC4)nc32)CC1)N1CCC(OC(F)(F)F)CC1] | (trans)-[4-(2-tetrahydropyran-4-yl- 3H-imidazo[4,5-b]pyridin-7- yl)cyclohexyl]-[4-(trifluoromethoxy)- 1-piperidyl]methanone |
| 194 | 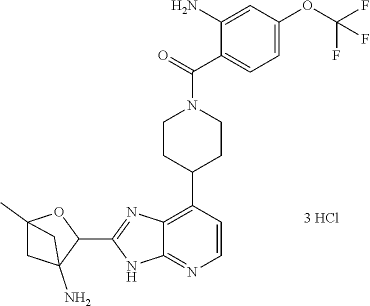 [SMILES: CC12CC(N)(C1)C(c1nc3c(C4CCN(C(=O)c5ccc(OC(F)(F)F)cc5N)CC4)ccnc3[nH]1)O2] | (rac)-[4-[2-(4-amino-1-methyl-2- oxabicyclo[2.1.1]hexan-3-yl)-3H- imidazo[4,5-b]pyridin-7-yl]-1- piperidyl]-[2-amino-4- (trifluoromethoxy)phenyl]methanone; hydrochloride |
| 195 | 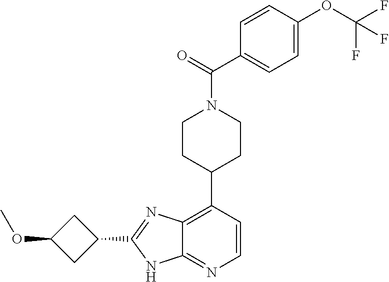 [SMILES: CO[C@H]1C[C@H](c2nc3c(C4CCN(C(=O)c5ccc(OC(F)(F)F)cc5)CC4)ccnc3[nH]2)C1] | (trans)-[4-[2-(3-methoxycyclobutyl)- 3H-imidazo[4,5-b]pyridin-7-yl]-1- piperidyl]-[4- (trifluoromethoxy)phenyl]methanone |
| 196 | 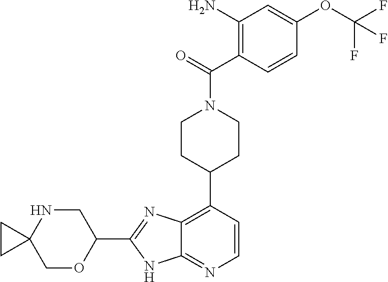 [SMILES: Nc1cc(OC(F)(F)F)ccc1C(=O)N1CCC(c2ccnc3[nH]c(C4CNC5(CC5)CO4)nc23)CC1] | (rac)-[2-amino-4- (trifluoromethoxy)phenyl]-[4-[2-(7- oxa-4-azaspiro[2.5]octan-6-yl)-3H- imidazo[4,5-b]pyridin-7-yl]-1- piperidyl]methanone |
| 197 | 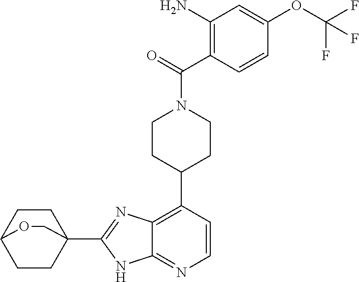 [SMILES: Nc1cc(OC(F)(F)F)ccc1C(=O)N1CCC(c2ccnc3[nH]c(C45CCC(CC4)OC5)nc23)CC1] | [2-amino-4- (trifluoromethoxy)phenyl]-[4-[2-(2- oxabicyclo[2.2.2]octan-4-yl)-3H- imidazo[4,5-b]pyridin-7-yl]-1- piperidyl]methanone |
| 198 | 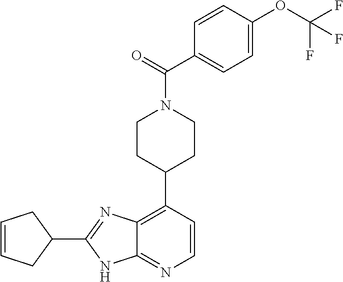 [SMILES: O=C(c1ccc(OC(F)(F)F)cc1)N1CCC(c2ccnc3[nH]c(C4CC=CC4)nc23)CC1] | [4-(2-cyclopent-3-en-1-yl-3H- imidazo[4,5-b]pyridin-7-yl)-1- piperidyl]-[4- (trifluoromethoxy)phenyl]methanone |
| 199 | 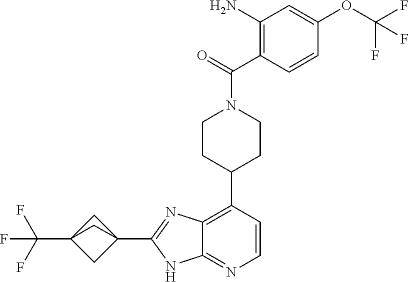 [SMILES: Nc1cc(OC(F)(F)F)ccc1C(=O)N1CCC(c2ccnc3[nH]c(C45CC(C(F)(F)F)(C4)C5)nc23)CC1] | [2-amino-4- (trifluoromethoxy)phenyl]-[4-[2-[3- (trifluoromethyl)-1- bicyclo[1.1.1]pentanyl]-3H- imidazo[4,5-b]pyridin-7-yl]-1- piperidyl]methanone |
| 200 | 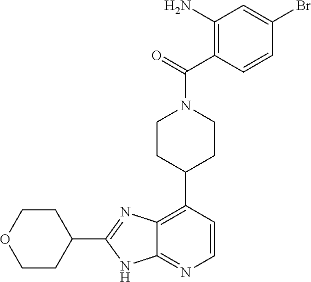 [SMILES: Nc1cc(Br)ccc1C(=O)N1CCC(c2ccnc3[nH]c(C4CCOCC4)nc23)CC1] | (2-amino-4-bromo-phenyl)-[4-(2- tetrahydropyran-4-yl-3H- imidazo[4,5-b]pyridin-7-yl)-1- piperidyl]methanone |
| 201 | 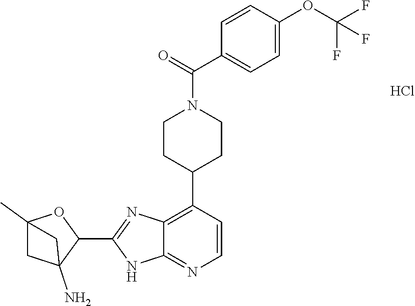 [SMILES: CC12CC(N)(C1)C(c1nc3c(C4CCN(C(=O)c5ccc(OC(F)(F)F)cc5)CC4)ccnc3[nH]1)O2 ; Cl] | (rac)-[4-[2-(4-amino-1-methyl-2- oxabicyclo[2.1.1]hexan-3-yl)-3H- imidazo[4,5-b]pyridin-7-yl]-1- piperidyl]-[4- (trifluoromethoxy)phenyl]methanone; hydrochloride |
| 202 | 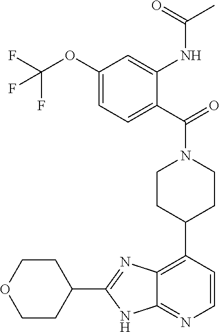 [SMILES: CC(=O)Nc1cc(OC(F)(F)F)ccc1C(=O)N1CCC(c2ccnc3[nH]c(C4CCOCC4)nc23)CC1] | N-[2-[4-(2-tetrahydropyran-4-yl-3H- imidazo[4,5-b]pyridin-7- yl)piperidine-1-carbonyl]-5- (trifluoromethoxy)phenyl]acetamide |
| 203 | 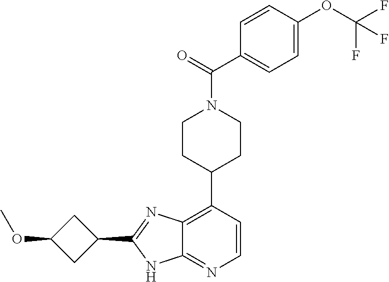 [SMILES: CO[C@H]1C[C@@H](c2nc3c(C4CCN(C(=O)c5ccc(OC(F)(F)F)cc5)CC4)ccnc3[nH]2)C1] | (trans)-[4-[2-(3-methoxycyclobutyl)- H-imidazo[4,5-b]pyridin-7-yl]-1- piperidyl]-[4- (trifluoromethoxy)phenyl]methanone |
| 204 | 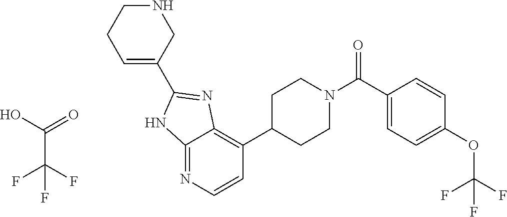 [SMILES: O=C(O)C(F)(F)F ; O=C(c1ccc(OC(F)(F)F)cc1)N1CCC(c2ccnc3[nH]c(C4=CCCNC4)nc23)CC1] | [4-[2-(1,2,3,6-tetrahydropyridin-5- yl)-3H-imidazo[4,5-b]pyridin-7-yl]- 1-piperidyl]-[4- (trifluoromethoxy)phenyl]methanone; 2,2,2-trifluoroacetic acid |
| 205 | 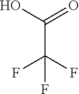 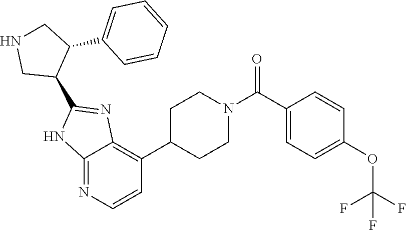 [SMILES: O=C(c1ccc(OC(F)(F)F)cc1)N1CCC(c2ccnc3[nH]c([C@H]4CNC[C@@H]4c4ccccc4)nc23)CC1] | [4-[2-[(3R,4S)-4-phenylpyrrolidin-3- yl]-3H-imidazo[4,5-b]pyridin-7-yl]- 1-piperidyl]-[4- (trifluoromethoxy)phenyl]methanone; 2,2,2-trifluoroacetic acid |
| 206 | 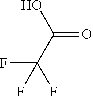 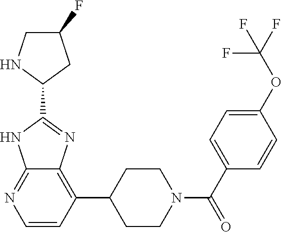 [SMILES: O=C(c1ccc(OC(F)(F)F)cc1)N1CCC(c2ccnc3[nH]c([C@H]4C[C@H](F)CN4)nc23)CC1] | [4-[2-[(2R,4S)-4-fluoropyrrolidin-2- yl]-3H-imidazo[4,5-b]pyridin-7-yl]- 1-piperidyl]-[4- (trifluoromethoxy)phenyl]methanone; 2,2,2-trifluoroacetic acid |
| 207 | 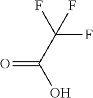 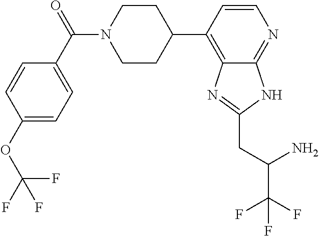 [SMILES: NC(Cc1nc2c(C3CCN(C(=O)c4ccc(OC(F)(F)F)cc4)CC3)ccnc2[nH]1)C(F)(F)F] | (rac)-[4-[2-(2-amino-3,3,3-trifluoro- propyl)-3H-imidazo[4,5-b]pyridin-7- yl]-1-piperidyl]-[4- (trifluoromethoxy)phenyl]methanone; 2,2,2-trifluoroacetic acid |
| 208 | 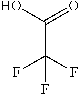 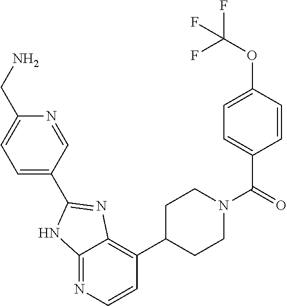 [SMILES: NCc1ccc(-c2nc3c(C4CCN(C(=O)c5ccc(OC(F)(F)F)cc5)CC4)ccnc3[nH]2)cn1] | [4-[2-[6-(aminomethyl)-3-pyridyl]- 3H-imidazo[4,5-b]pyridin-7-yl]-1- piperidyl]-[4- (trifluoromethoxy)phenyl]methanone; 2,2,2-trifluoroacetic acid |
| 209 | 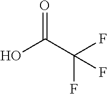 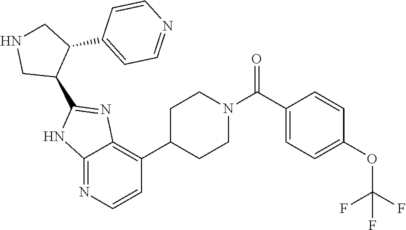 [SMILES: O=C(c1ccc(OC(F)(F)F)cc1)N1CCC(c2ccnc3[nH]c([C@H]4CNC[C@@H]4c4ccncc4)nc23)CC1] | [4-[2-[rac-(3R,4S)-4-(4- pyridyl)pyrrolidin-3-yl]-3H- imidazo[4,5-b]pyridin-7-yl]-1- piperidyl]-[4- (trifluoromethoxy)phenyl]methanone; 2,2,2-trifluoroacetic acid |
| 210 | 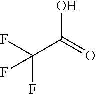 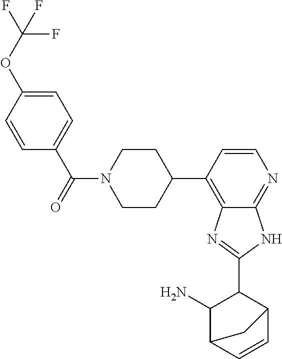 [SMILES: NC1C2C=CC(C2)C1c1nc2c(C3CCN(C(=O)c4ccc(OC(F)(F)F)cc4)CC3)ccnc2[nH]1] | [4-[2-(3-amino-2-bicyclo[2.2.1]hept- 5-enyl)-3H-imidazo[4,5-b]pyridin-7- yl]-1-piperidyl]-[4- (trifluoromethoxy)phenyl]methanone; 2,2,2-trifluoroacetic acid |
| 211 | 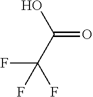 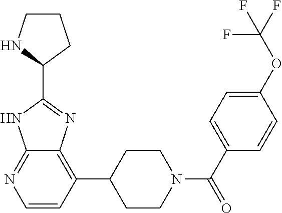 [SMILES: O=C(c1ccc(OC(F)(F)F)cc1)N1CCC(c2ccnc3[nH]c([C@@H]4CCCN4)nc23)CC1] | [4-[2-[(2S)-pyrrolidin-2-yl]-3H- imidazo[4,5-b]pyridin-7-yl]-1- piperidyl]-[4- (trifluoromethoxy)phenyl]methanone; 2,2,2-trifluoroacetic acid |
| 212 | 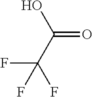 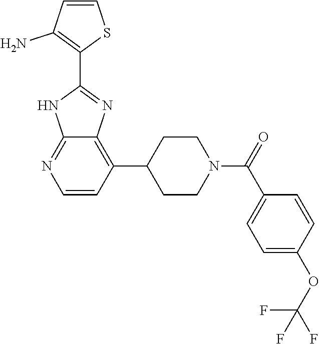 [SMILES: Nc1ccsc1-c1nc2c(C3CCN(C(=O)c4ccc(OC(F)(F)F)cc4)CC3)ccnc2[nH]1] | [4-[2-(3-amino-2-thienyl)-3H- imidazo[4,5-b]pyridin-7-yl]-1- piperidyl]-[4- (trifluoromethoxy)phenyl]methanone; 2,2,2-trifluoroacetic acid |
| 213 | 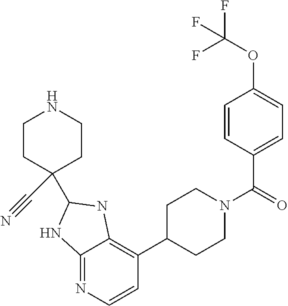 [SMILES: N#CC1(C2Nc3nccc(C4CCN(C(=O)c5ccc(OC(F)(F)F)cc5)CC4)c3N2)CCNCC1] 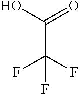 | 4-[7-[1-[4- (trifluoromethoxy)benzoyl]-4- piperidyl]-3H-imidazo[4,5-b]pyridin- 2-yl]piperidine-4-carbonitrile;2,2,2- trifluoroacetic acid |
| 214 | 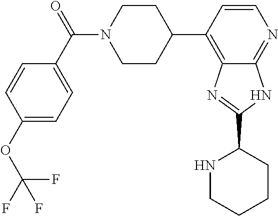 [SMILES: O=C(c1ccc(OC(F)(F)F)cc1)N1CCC(c2ccnc3[nH]c([C@H]4CCCCN4)nc23)CC1] 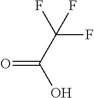 | [4-[2-[(2R)-2-piperidyl]-3H- imidazo[4,5-b]pyridin-7-yl]-1- piperidyl]-[4- (trifluoromethoxy)phenyl]methanone; 2,2,2-trifluoroacetic acid |
| 215 | 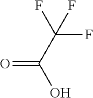 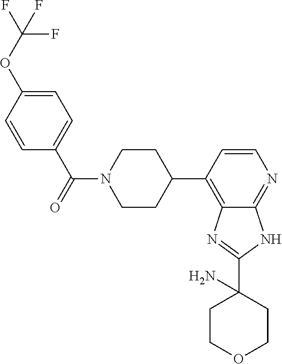 [SMILES: NC1(c2nc3c(C4CCN(C(=O)c5ccc(OC(F)(F)F)cc5)CC4)ccnc3[nH]2)CCOCC1] | [4-[2-(4-aminotetrahydropyran-4-yl)- 3H-imidazo[4,5-b]pyridin-7-yl]-1- piperidyl]-[4- (trifluoromethoxy)phenyl]methanone; 2,2,2-trifluoroacetic acid |
| 216 | 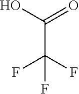 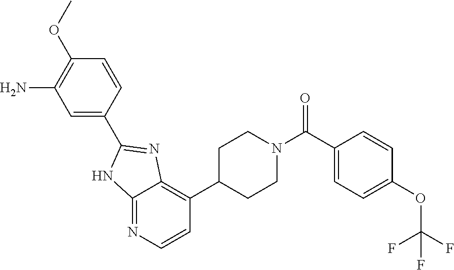 [SMILES: COc1ccc(-c2nc3c(C4CCN(C(=O)c5ccc(OC(F)(F)F)cc5)CC4)ccnc3[nH]2)cc1N] | [4-[2-(3-amino-4-methoxy-phenyl)- 3H-imidazo[4,5-b]pyridin-7-yl]-1- piperidyl]-[4- (trifluoromethoxy)phenyl]methanone; 2,2,2-trifluoroacetic acid |
| 217 | 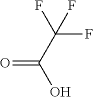 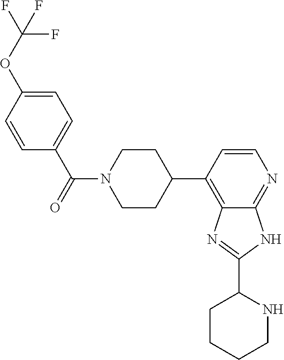 [SMILES: O=C(c1ccc(OC(F)(F)F)cc1)N1CCC(c2ccnc3[nH]c(C4CCCCN4)nc23)CC1] | (rac)-[4-[2-(2-piperidyl)-3H- imidazo[4,5-b]pyridin-7-yl]-1- piperidyl]-[4- (trifluoromethoxy)phenyl]methanone; 2,2,2-trifluoroacetic acid |
| 218 | 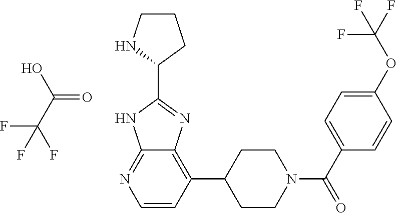 [SMILES: O=C(O)C(F)(F)F ; O=C(c1ccc(OC(F)(F)F)cc1)N1CCC(c2ccnc3[nH]c([C@H]4CCCN4)nc23)CC1] | [4-[2-[(2R)-pyrrolidin-2-yl]-3H- imidazo[4,5-b]pyridin-7-yl]-1- piperidyl]-[4- (trifluoromethoxy)phenyl]methanone; 2,2,2-trifluoroacetic acid |
| 219 | 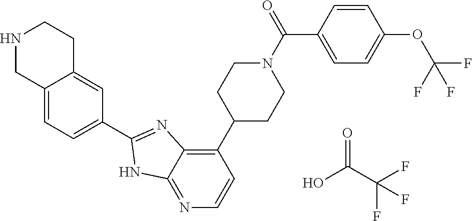 [SMILES: O=C(c1ccc(OC(F)(F)F)cc1)N1CCC(c2ccnc3[nH]c(-c4ccc5c(c4)CCNC5)nc23)CC1 ; O=C(O)C(F)(F)F] | [4-[2-(1,2,3,4-tetrahydroisoquinolin- 6-yl)-3H-imidazo[4,5-b]pyridin-7- yl]-1-piperidyl]-[4- (trifluoromethoxy)phenyl]methanone; 2,2,2-trifluoroacetic acid |
| 220 | 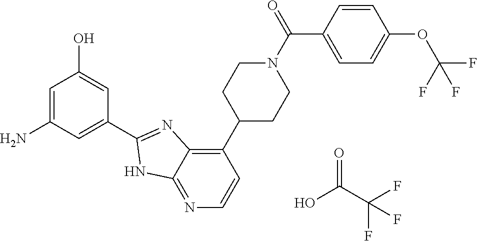 [SMILES: Nc1cc(O)cc(-c2nc3c(C4CCN(C(=O)c5ccc(OC(F)(F)F)cc5)CC4)ccnc3[nH]2)c1 ; O=C(O)C(F)(F)F] | [4-[2-(3-amino-5-hydroxy-phenyl)- 3H-imidazo[4,5-b]pyridin-7-yl]-1- piperidyl]-[4- (trifluoromethoxy)phenyl]methanone; 2,2,2-trifluoroacetic acid |
| 221 | 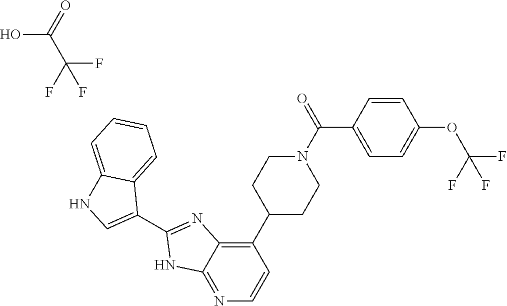 | [4-[2-(1H-indol-3-yl)-3H- imidazo[4,5-b]pyridin-7-yl]-1- piperidyl]-[4- (trifluoromethoxy)phenyl]methanone; 2,2,2-trifluoroacetic acid |
| 222 | 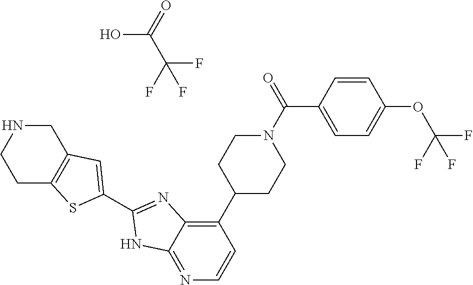 [SMILES: O=C(c1ccc(OC(F)(F)F)cc1)N1CCC(c2ccnc3[nH]c(-c4cc5c(s4)CCNC5)nc23)CC1 ; O=C(O)C(F)(F)F] | [4-[2-(4,5,6,7-tetrahydrothieno[3,2- c]pyridin-2-yl)-3H-imidazo[4,5- b]pyridin-7-yl]-1-piperidyl]-[4- (trifluoromethoxy)phenyl]methanone; 2,2,2-trifluoroacetic acid |
| 223 | 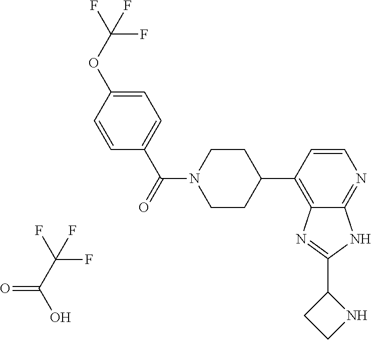 [SMILES: O=C(O)C(F)(F)F ; O=C(c1ccc(OC(F)(F)F)cc1)N1CCC(c2ccnc3[nH]c(C4CCN4)nc23)CC1] | (rac)-[4-[2-(azetidin-2-yl)-3H- imidazo[4,5-b]pyridin-7-yl]-1- piperidyl]-[4- (trifluoromethoxy)phenyl]methanone; 2,2,2-trifluoroacetic acid |
| 224 | 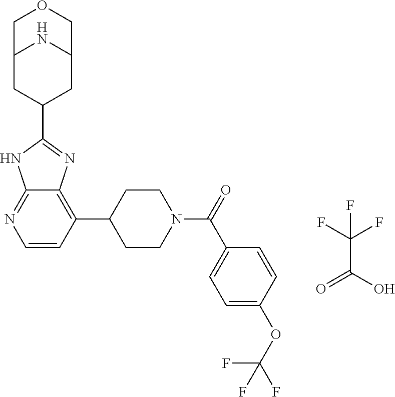 [SMILES: O=C(c1ccc(OC(F)(F)F)cc1)N1CCC(c2ccnc3[nH]c(C4CC5COCC(C4)N5)nc23)CC1 ; O=C(O)C(F)(F)F] | (rac)-[4-[2-(3-oxa-9- azabicyclo[3.3.1]nonan-7-yl)-3H- imidazo[4,5-b]pyridin-7-yl]-1- piperidyl]-[4- (trifluoromethoxy)phenyl]methanone; 2,2,2-trifluoroacetic acid |
| 225 | 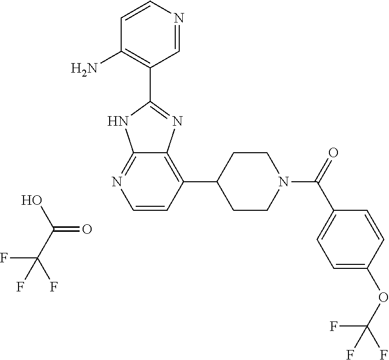 [SMILES: O=C(O)C(F)(F)F ; Nc1ccncc1-c1nc2c(C3CCN(C(=O)c4ccc(OC(F)(F)F)cc4)CC3)ccnc2[nH]1] | [4-[2-(4-amino-3-pyridyl)-3H- imidazo[4,5-b]pyridin-7-yl]-1- piperidyl]-[4- (trifluoromethoxy)phenyl]methanone; 2,2,2-trifluoroacetic acid |
| 226 | 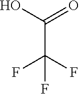 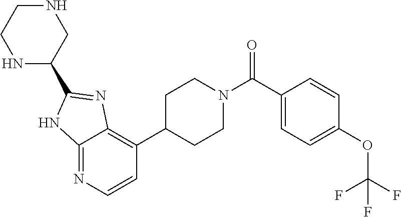 [SMILES: O=C(c1ccc(OC(F)(F)F)cc1)N1CCC(c2ccnc3[nH]c([C@@H]4CNCCN4)nc23)CC1] | [4-[2-[(2R)-piperazin-2-yl]-3H- imidazo[4,5-b]pyridin-7-yl]-1- piperidyl]-[4- (trifluoromethoxy)phenyl]methanone; 2,2,2-trifluoroacetic acid |
| 227 | 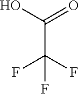 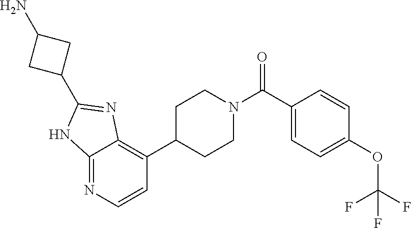 [SMILES: NC1CC(c2nc3c(C4CCN(C(=O)c5ccc(OC(F)(F)F)cc5)CC4)ccnc3[nH]2)C1] | [4-[2-(3-aminocyclobutyl)-3H- imidazo[4,5-b]pyridin-7-yl]-1- piperidyl]-[4- (trifluoromethoxy)phenyl]methanone; 2,2,2-trifluoroacetic acid |
| 228 | 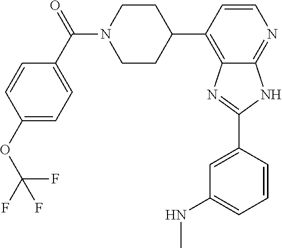 [SMILES: CNc1cccc(-c2nc3c(C4CCN(C(=O)c5ccc(OC(F)(F)F)cc5)CC4)ccnc3[nH]2)c1] 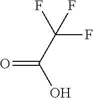 | [4-[2-[3-(methylamino)phenyl]-3H- imidazo[4,5-b]pyridin-7-yl]-1- piperidyl]-[4- (trifluoromethoxy)phenyl]methanone; 2,2,2-trifluoroacetic acid |
| 229 | 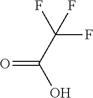 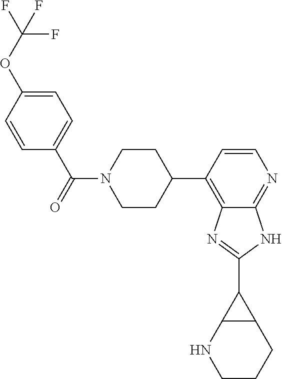 [SMILES: O=C(c1ccc(OC(F)(F)F)cc1)N1CCC(c2ccnc3[nH]c(C4C5CCCNC54)nc23)CC1] | (rac)-[4-[2-(2- azabicyclo[4.1.0]heptan-7-yl)-3H- imidazo[4,5-b]pyridin-7-yl]-1- piperidyl]-[4- (trifluoromethoxy)phenyl]methanone; 2,2,2-trifluoroacetic acid |
| 230 | 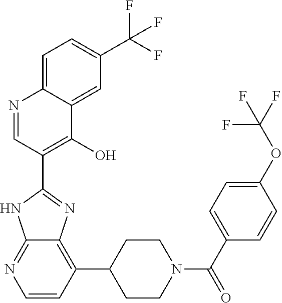 [SMILES: O=C(c1ccc(OC(F)(F)F)cc1)N1CCC(c2ccnc3[nH]c(-c4cnc5ccc(C(F)(F)F)cc5c4O)nc23)CC1] | [4-[2-[4-hydroxy-6-(trifluoromethyl)- 3-quinolyl]-3H-imidazo[4,5- b]pyridin-7-yl]-1-piperidyl]-[4- (trifluoromethoxy)phenyl]methanone |
| 231 | 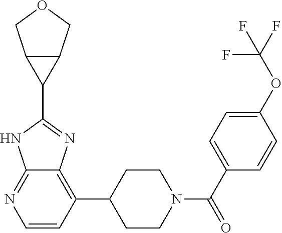 [SMILES: O=C(c1ccc(OC(F)(F)F)cc1)N1CCC(c2ccnc3[nH]c(C4C5COCC54)nc23)CC1] | (rac)-[4-[2-(3- oxabicyclo[3.1.0]hexan-6-yl)-3H- imidazo[4,5-b]pyridin-7-yl]-1- piperidyl]-[4- (trifluoromethoxy)phenyl]methanone |
| 232 | 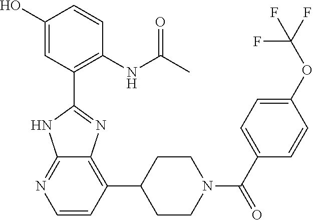 [SMILES: CC(=O)Nc1ccc(O)cc1-c1nc2c(C3CCN(C(=O)c4ccc(OC(F)(F)F)cc4)CC3)ccnc2[nH]1] | N-[4-hydroxy-2-[7-[1-[4- (trifluoromethoxy)benzoyl]-4- piperidyl]-3H-imidazo[4,5-b]pyridin- 2-yl]phenyl]acetamide |
| 233 | 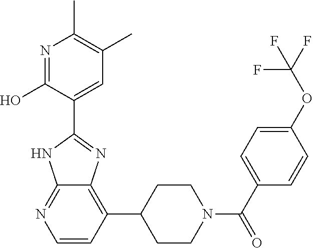 [SMILES: Cc1cc(-c2nc3c(C4CCN(C(=O)c5ccc(OC(F)(F)F)cc5)CC4)ccnc3[nH]2)c(O)nc1C] | 5,6-dimethyl-3-[7-[1-[4- (trifluoromethoxy)benzoyl]-4- piperidyl]-3H-imidazo[4,5-b]pyridin- 2-yl]-1H-pyridin-2-one |
| 234 | 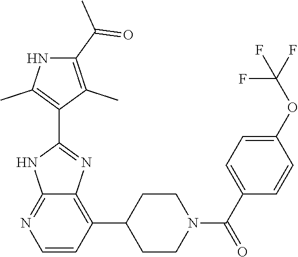 [SMILES: CC(=O)c1[nH]c(C)c(-c2nc3c(C4CCN(C(=O)c5ccc(OC(F)(F)F)cc5)CC4)ccnc3[nH]2)c1C] | 1-[3,5-dimethyl-4-[7-[1-[4- (trifluoromethoxy)benzoyl]-4- piperidyl]-3H-imidazo[4,5-b]pyridin- 2-yl]-1H-pyrrol-2-yl]ethanone |
| 235 | 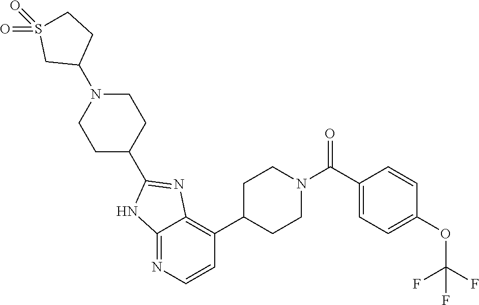 [SMILES: O=C(c1ccc(OC(F)(F)F)cc1)N1CCC(c2ccnc3[nH]c(C4CCN(C5CCS(=O)(=O)C5)CC4)nc23)CC1] | [4-[2-[1-(1,1-dioxothiolan-3-yl)-4- piperidyl]-3H-imidazo[4,5-b]pyridin- 7-yl]-1-piperidyl]-[4- (trifluoromethoxy)phenyl]methanone |
| 236 | 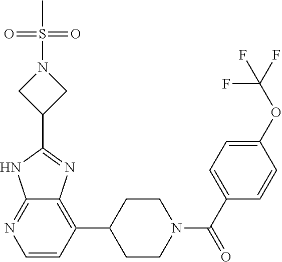 [SMILES: CS(=O)(=O)N1CC(c2nc3c(C4CCN(C(=O)c5ccc(OC(F)(F)F)cc5)CC4)ccnc3[nH]2)C1] | [4-[2-(1-methylsulfonylazetidin-3- yl)-3H-imidazo[4,5-b]pyridin-7-yl]- 1-piperidyl]-[4- (trifluoromethoxy)phenyl]methanone |
| 237 | 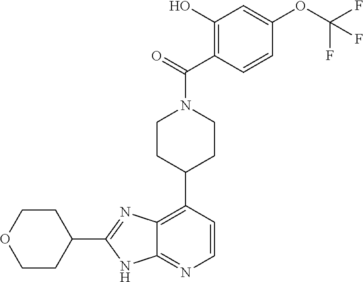 [SMILES: O=C(c1ccc(OC(F)(F)F)cc1O)N1CCC(c2ccnc3[nH]c(C4CCOCC4)nc23)CC1] | [2-hydroxy-4- (trifluoromethoxy)phenyl]-[4-(2- tetrahydropyran-4-yl-3H- imidazo[4,5-b]pyridin-7-yl)-1- piperidyl]methanone |
| 238 | 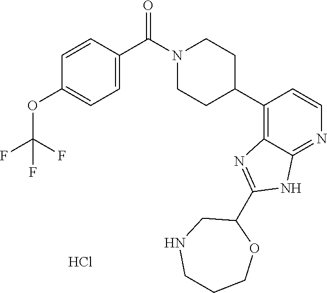 [SMILES: O=C(c1ccc(OC(F)(F)F)cc1)N1CCC(c2ccnc3[nH]c(C4CNCCCO4)nc23)CC1 ; Cl] | (rac)-[4-[2-(1,4-oxazepan-2-yl)-3H- imidazo[4,5-b]pyridin-7-yl]-1- piperidyl]-[4- (trifluoromethoxy)phenyl]methanone; hydrochloride |
| 239 | 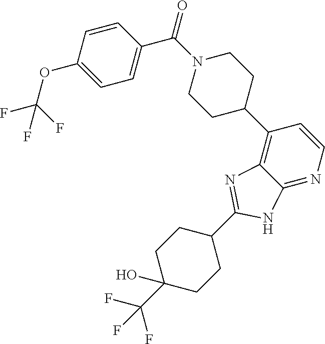 [SMILES: O=C(c1ccc(OC(F)(F)F)cc1)N1CCC(c2ccnc3[nH]c(C4CCC(O)(C(F)(F)F)CC4)nc23)CC1] | [4-[2-[4-hydroxy-4- (trifluoromethyl)cyclohexyl]-3H- imidazo[4,5-b]pyridin-7-yl]-1- piperidyl]-[4- (trifluoromethoxy)phenyl]methanone |
| 240 | 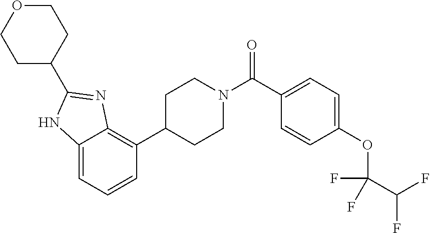 [SMILES: O=C(c1ccc(OC(F)(F)C(F)F)cc1)N1CCC(c2cccc3[nH]c(C4CCOCC4)nc23)CC1] | [4-(1,1,2,2- tetrafluoroethoxy)phenyl]-[4-(2- tetrahydropyran-4-yl-3H- imidazo[4,5-b]pyridin-7-yl)-1- piperidyl]methanone |
| 241 | 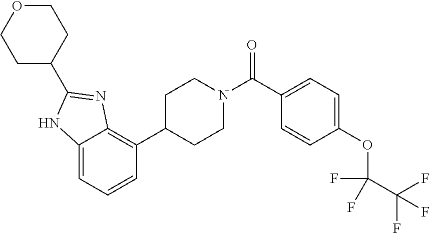 [SMILES: O=C(c1ccc(OC(F)(F)C(F)(F)F)cc1)N1CCC(c2cccc3[nH]c(C4CCOCC4)nc23)CC1] | [4-(1,1,2,2,2- pentafluoroethoxy)phenyl]-[4-(2- tetrahydropyran-4-yl-3H- imidazo[4,5-b]pyridin-7-yl)-1- piperidyl]methanone |
| 242 | 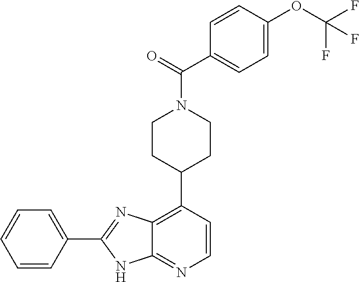 [SMILES: O=C(c1ccc(OC(F)(F)F)cc1)N1CCC(c2ccnc3[nH]c(-c4ccccc4)nc23)CC1] | [4-(2-phenyl-3H-imidazo[4,5- b]pyridin-7-yl)-1-piperidyl]-[4- (trifluoromethoxy)phenyl]methanone |
| 243 | 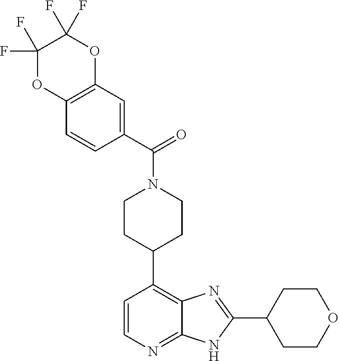 [SMILES: O=C(c1ccc2c(c1)OC(F)(F)C(F)(F)O2)N1CCC(c2ccnc3[nH]c(C4CCOCC4)nc23)CC1] | (2,2,3,3-tetrafluoro-1,4-benzodioxin- 6-yl)-[4-(2-tetrahydropyran-4-yl-3H- imidazo[4,5-b]pyridin-7-yl)-1- piperidyl]methanone |
| 244 | 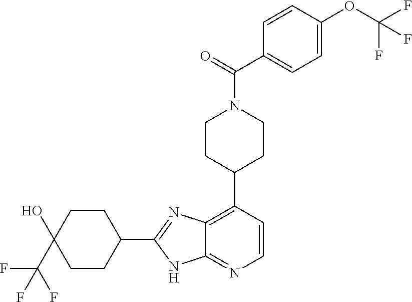 [SMILES: O=C(c1ccc(OC(F)(F)F)cc1)N1CCC(c2ccnc3[nH]c(C4CCC(O)(C(F)(F)F)CC4)nc23)CC1] | [4-[2-[4-hydroxy-4- (trifluoromethyl)cyclohexyl]-3H- imidazo[4,5-b]pyridin-7-yl]-1- piperidyl]-[4- (trifluoromethoxy)phenyl]methanone |
| 245 | 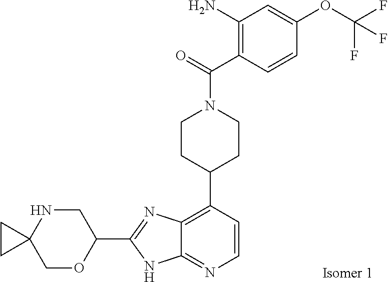 [SMILES: Nc1cc(OC(F)(F)F)ccc1C(=O)N1CCC(c2ccnc3[nH]c(C4CNC5(CC5)CO4)nc23)CC1] | [2-amino-4- (trifluoromethoxy)phenyl]-[4-[2-[7- oxa-4-azaspiro[2.5]octan-6-yl]-3H- imidazo[4,5-b]pyridin-7-yl]-1- piperidyl]methanone, Isomer 1 |
| 246 | 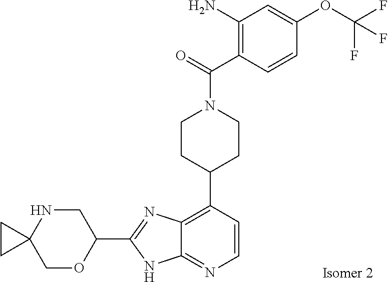 [SMILES: Nc1cc(OC(F)(F)F)ccc1C(=O)N1CCC(c2ccnc3[nH]c(C4CNC5(CC5)CO4)nc23)CC1] | [2-amino-4- (trifluoromethoxy)phenyl]-[4-[2-[7- oxa-4-azaspiro[2.5]octan-6-yl]-3H- imidazo[4,5-b]pyridin-7-yl]-1- piperidyl]methanone, Isomer 2 |
| 247 | 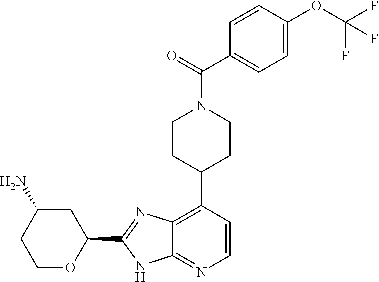 [SMILES: N[C@H]1CCO[C@H](c2nc3c(C4CCN(C(=O)c5ccc(OC(F)(F)F)cc5)CC4)ccnc3[nH]2)C1] | [4-[2-[rac-(1S,3S)-3- aminocyclohexyl]-3H-imidazo[4,5- b]pyridin-7-yl]-1-piperidyl]-[4- (trifluoromethoxy)phenyl]methanone |
| 248 | 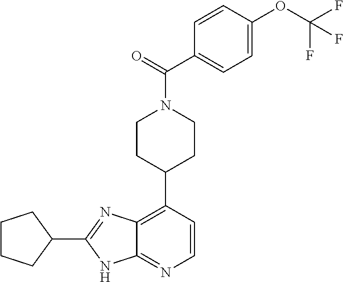 [SMILES: O=C(c1ccc(OC(F)(F)F)cc1)N1CCC(c2ccnc3[nH]c(C4CCCC4)nc23)CC1] | [4-(2-cyclopentyl-3H-imidazo[4,5- b]pyridin-7-yl)-1-piperidyl]-[4- (trifluoromethoxy)phenyl]methanone |
| 249 | 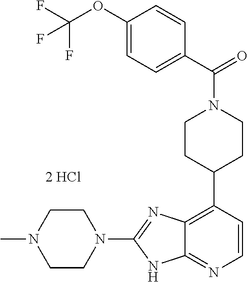 [SMILES: CN1CCN(c2nc3c(C4CCN(C(=O)c5ccc(OC(F)(F)F)cc5)CC4)ccnc3[nH]2)CC1] | [4-[2-(4-methylpiperazin-1-yl)-3H- imidazo[4,5-b]pyridin-7-yl]-1- piperidyl]-[4- (trifluoromethoxy)phenyl]methanone hydrochloride |
| 250 | 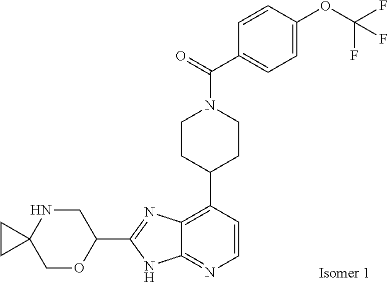 [SMILES: O=C(c1ccc(OC(F)(F)F)cc1)N1CCC(c2ccnc3[nH]c(C4CNC5(CC5)CO4)nc23)CC1] | [4-[2-[7-oxa-4-azaspiro[2.5]octan-6- yl]-3H-imidazo[4,5-b]pyridin-7-yl]- 1-piperidyl]-[4- (trifluoromethoxy)phenyl]methanone, Isomer 1 |
| 251 | 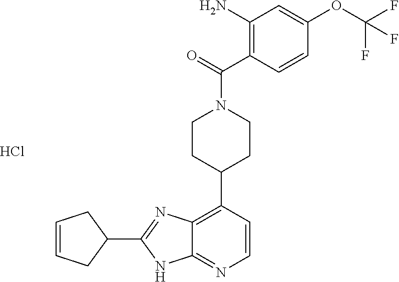 [SMILES: Cl ; Nc1cc(OC(F)(F)F)ccc1C(=O)N1CCC(c2ccnc3[nH]c(C4CC=CC4)nc23)CC1] | [2-amino-4- (trifluoromethoxy)phenyl]-[4-(2- cyclopent-3-en-1-yl-3H-imidazo[4,5- b]pyridin-7-yl)-1- piperidyl]methanone;hydrochloride |
| 252 | 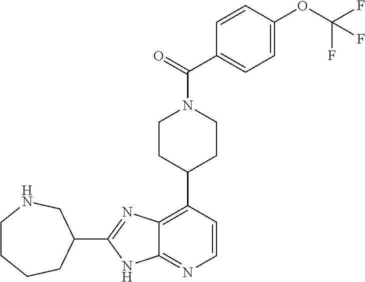 [SMILES: O=C(c1ccc(OC(F)(F)F)cc1)N1CCC(c2ccnc3[nH]c(C4CCCCNC4)nc23)CC1] | (rac)-[4-[2-(azepan-3-yl)-3H- imidazo[4,5-b]pyridin-7-yl]-1- piperidyl]-[4- (trifluoromethoxy)phenyl]methanone |
| 253 | 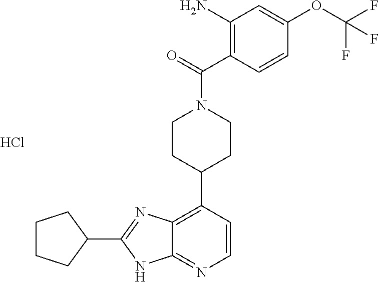 [SMILES: Cl ; Nc1cc(OC(F)(F)F)ccc1C(=O)N1CCC(c2ccnc3[nH]c(C4CCCC4)nc23)CC1] | [2-amino-4- (trifluoromethoxy)phenyl]-[4-(2- cyclopentyl-3H-imidazo[4,5- b]pyridin-7-yl)-1- piperidyl]methanone;hydrochloride |
| 254 | 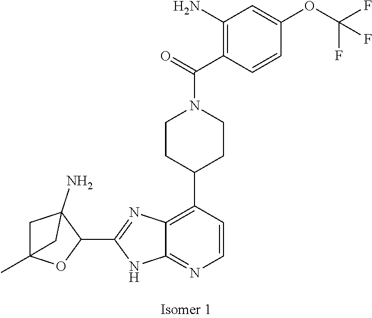 [SMILES: CC12CC(N)(C1)C(c1nc3c(C4CCN(C(=O)c5ccc(OC(F)(F)F)cc5N)CC4)ccnc3[nH]1)O2] | [4-[2-[4-amino-1-methyl-2- oxabicyclo[2.1.1]hexan-3-yl]-3H- imidazo[4,5-b]pyridin-7-yl]-1- piperidyl]-[2-amino-4- (trifluoromethoxy)phenyl]methanone, Isomer 1 |
| 255 | 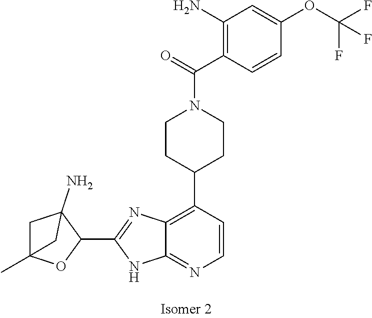 [SMILES: CC12CC(N)(C1)C(c1nc3c(C4CCN(C(=O)c5ccc(OC(F)(F)F)cc5N)CC4)ccnc3[nH]1)O2] | [4-[2-[4-amino-1-methyl-2- oxabicyclo[2.1.1]hexan-3-yl]-3H- imidazo[4,5-b]pyridin-7-yl]-1- piperidyl]-[2-amino-4- (trifluoromethoxy)phenyl]methanone, Isomer 2 |
| 256 | 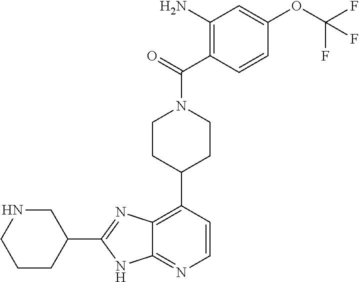 [SMILES: Nc1cc(OC(F)(F)F)ccc1C(=O)N1CCC(c2ccnc3[nH]c(C4CCCNC4)nc23)CC1] | (rac)-[2-amino-4- (trifluoromethoxy)phenyl]-[4-[2-(3- piperidyl)-3H-imidazo[4,5-b]pyridin- 7-yl]-1-piperidyl]methanone |
| 257 | 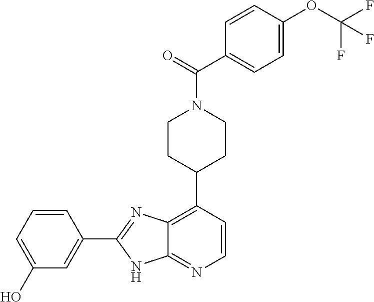 [SMILES: O=C(c1ccc(OC(F)(F)F)cc1)N1CCC(c2ccnc3[nH]c(-c4cccc(O)c4)nc23)CC1] | [4-[2-(3-hydroxyphenyl)-3H- imidazo[4,5-b]pyridin-7-yl]-1- piperidyl]-[4- (trifluoromethoxy)phenyl]methanone |
| 258 | 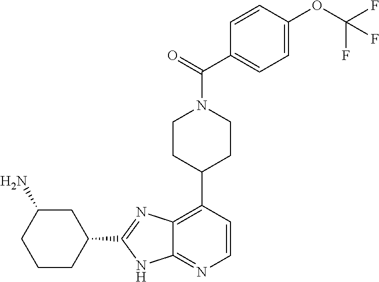 [SMILES: N[C@H]1CCC[C@@H](c2nc3c(C4CCN(C(=O)c5ccc(OC(F)(F)F)cc5)CC4)ccnc3[nH]2)C1] | [4-[2-[rac-(1R,3S)-3- aminocyclohexyl]-3H-imidazo[4,5- b]pyridin-7-yl]-1-piperidyl]-[4- (trifluoromethoxy)phenyl]methanone |
| 259 | 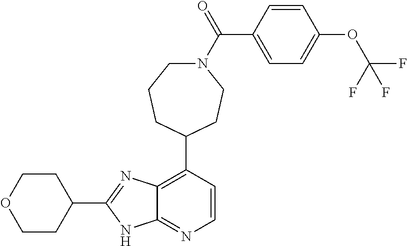 [SMILES: O=C(c1ccc(OC(F)(F)F)cc1)N1CCCC(c2ccnc3[nH]c(C4CCOCC4)nc23)CC1] | (rac)-[4-(2-tetrahydropyran-4-yl-3H- imidazo[4,5-b]pyridin-7-yl)azepan-1- yl]-[4- (trifluoromethoxy)phenyl]methanone |
| 260 | 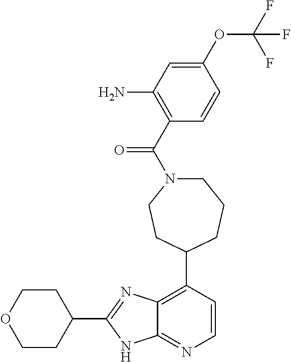 [SMILES: Nc1cc(OC(F)(F)F)ccc1C(=O)N1CCCC(c2ccnc3[nH]c(C4CCOCC4)nc23)CC1] | (rac)-[2-amino-4- (trifluoromethoxy)phenyl]-[4-(2- tetrahydropyran-4-yl-3H- imidazo[4,5-b]pyridin-7-yl)azepan-1- yl]methanone |
| 261 | 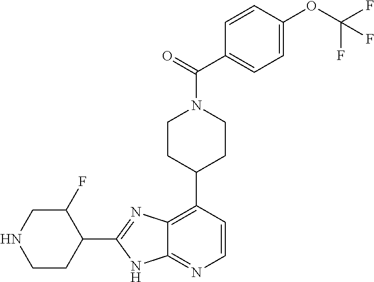 [SMILES: O=C(c1ccc(OC(F)(F)F)cc1)N1CCC(c2ccnc3[nH]c(C4CCNCC4F)nc23)CC1] | (rac)-[4-[2-(3-fluoro-4-piperidyl)-3H- imidazo[4,5-b]pyridin-7-yl]-1- piperidyl]-[4- (trifluoromethoxy)phenyl]methanone |
| 262 | 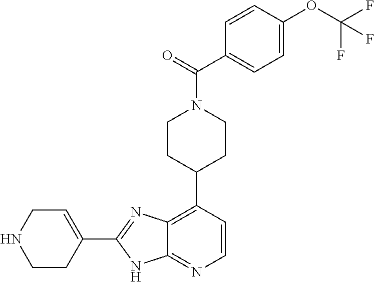 [SMILES: O=C(c1ccc(OC(F)(F)F)cc1)N1CCC(c2ccnc3[nH]c(C4=CCNCC4)nc23)CC1] | [4-[2-(1,2,3,6-tetrahydropyridin-4- yl)-3H-imidazo[4,5-b]pyridin-7-yl]- 1-piperidyl]-[4- (trifluoromethoxy)phenyl]methanone |
| 263 | 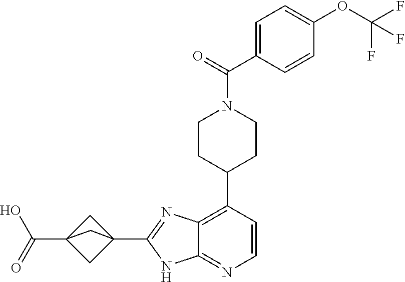 [SMILES: O=C(c1ccc(OC(F)(F)F)cc1)N1CCC(c2ccnc3[nH]c(C45CC(C(=O)O)(C4)C5)nc23)CC1] | 3-[7-[1-[4- (trifluoromethoxy)benzoyl]-4- piperidyl]-3H-imidazo[4,5-b]pyridin- 2-yl]bicyclo[1.1.1]pentane-1- carboxylic acid |
| 264 | 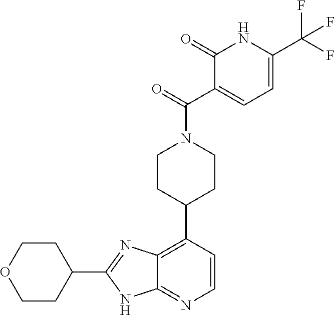 [SMILES: O=C(c1ccc(C(F)(F)F)[nH]c1=O)N1CCC(c2ccnc3[nH]c(C4CCOCC4)nc23)CC1] | 3-[4-(2-tetrahydropyran-4-yl-3H- imidazo[4,5-b]pyridin-7- yl)piperidine-1-carbonyl]-6- (trifluoromethyl)-1H-pyridin-2-one |
| 265 | 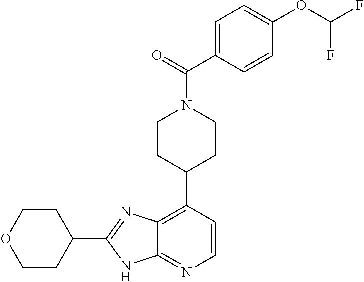 [SMILES: O=C(c1ccc(OC(F)F)cc1)N1CCC(c2ccnc3[nH]c(C4CCOCC4)nc23)CC1] | [4-(difluoromethoxy)phenyl]-[4-(2- tetrahydropyran-4-yl-3H- imidazo[4,5-b]pyridin-7-yl)-1- piperidyl]methanone |
| 266 | 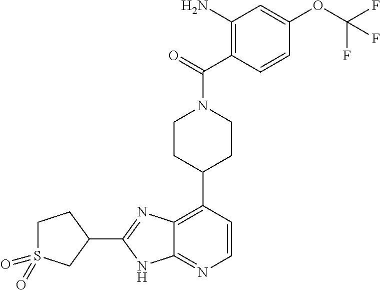 [SMILES: Nc1cc(OC(F)(F)F)ccc1C(=O)N1CCC(c2ccnc3[nH]c(C4CCS(=O)(=O)C4)nc23)CC1] Isomer 1 | [2-amino-4- (trifluoromethoxy)phenyl]-[4-[2-[1,1- dioxothiolan-3-yl]-3H-imidazo[4,5- b]pyridin-7-yl]-1- piperidyl]methanone, Isomer 1 |
| 267 | 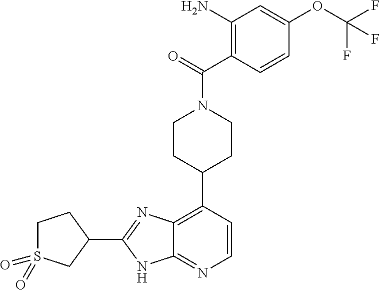 [SMILES: Nc1cc(OC(F)(F)F)ccc1C(=O)N1CCC(c2ccnc3[nH]c(C4CCS(=O)(=O)C4)nc23)CC1] Isomer 2 | [2-amino-4- (trifluoromethoxy)phenyl]-[4-[2-[1,1- dioxothiolan-3-yl]-3H-imidazo[4,5- b]pyridin-7-yl]-1- piperidyl]methanone, Isomer 2 |
| 268 | 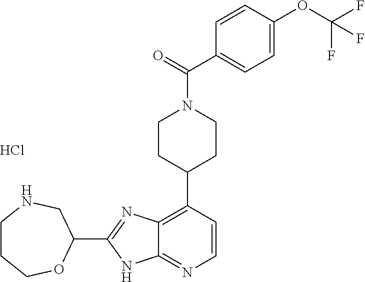 [SMILES: O=C(c1ccc(OC(F)(F)F)cc1)N1CCC(c2ccnc3[nH]c(C4CNCCCO4)nc23)CC1 ; Cl] Isomer 1 | [4-[2-[1,4-oxazepan-2-yl]-3H- imidazo[4,5-b]pyridin-7-yl]-1- piperidyl]-[4- (trifluoromethoxy)phenyl]methanone; hydrochloride, Isomer 1 |
| 269 | 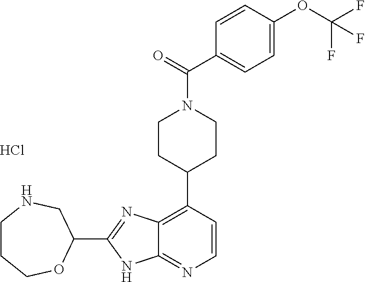 [SMILES: O=C(c1ccc(OC(F)(F)F)cc1)N1CCC(c2ccnc3[nH]c(C4CNCCCO4)nc23)CC1 ; Cl] Isomer 2 | [4-[2-[1,4-oxazepan-2-yl]-3H- imidazo[4,5-b]pyridin-7-yl]-1- piperidyl]-[4- (trifluoromethoxy)phenyl]methanone; hydrochloride, Isomer 2 |
| 270 | 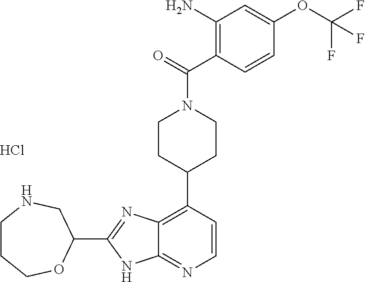 [SMILES: Nc1cc(OC(F)(F)F)ccc1C(=O)N1CCC(c2ccnc3[nH]c(C4CNCCCO4)nc23)CC1 ; Cl] Isomer 1 | [2-amino-4- (trifluoromethoxy)phenyl]-[4-[2-[1,4- oxazepan-2-yl]-3H-imidazo[4,5- b]pyridin-7-yl]-1- piperidyl]methanone;hydrochloride, Isomer 1 |
| 271 | 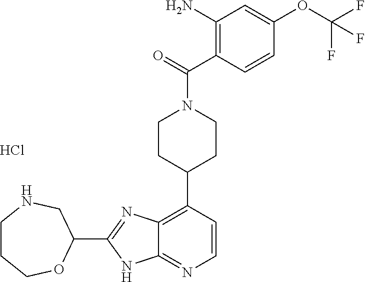 [SMILES: Nc1cc(OC(F)(F)F)ccc1C(=O)N1CCC(c2ccnc3[nH]c(C4CNCCCO4)nc23)CC1 ; Cl] Isomer 2 | [2-amino-4- (trifluoromethoxy)phenyl]-[4-[2-[1,4- oxazepan-2-yl]-3H-imidazo[4,5- b]pyridin-7-yl]-1- piperidyl]methanone;hydrochloride, Isomer 2 |
| 272 | 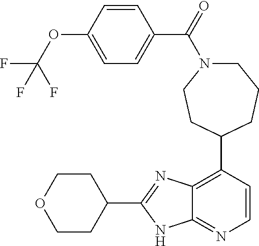 [SMILES: O=C(c1ccc(OC(F)(F)F)cc1)N1CCCC(c2ccnc3[nH]c(C4CCOCC4)nc23)CC1] Isomer 1 | [4-(2-tetrahydropyran-4-yl-3H- imidazo[4,5-b]pyridin-7-yl)azepan-1- yl]-[4- (trifluoromethoxy)phenyl]methanone, Isomer 1 |
| 273 | 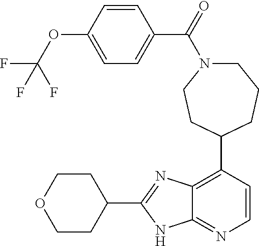 [SMILES: O=C(c1ccc(OC(F)(F)F)cc1)N1CCCC(c2ccnc3[nH]c(C4CCOCC4)nc23)CC1] Isomer 2 | [4-(2-tetrahydropyran-4-yl-3H- imidazo[4,5-b]pyridin-7-yl)azepan-1- yl]-[4- (trifluoromethoxy)phenyl]methanone, Isomer 2 |
| 274 | 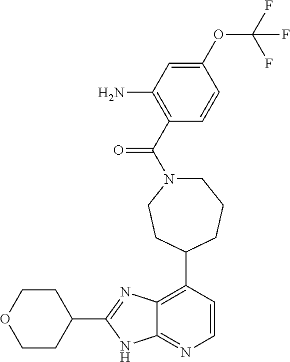 [SMILES: Nc1cc(OC(F)(F)F)ccc1C(=O)N1CCCC(c2ccnc3[nH]c(C4CCOCC4)nc23)CC1] Isomer 1 | [2-amino-4- (trifluoromethoxy)phenyl]-[4-(2- tetrahydropyran-4-yl-3H- imidazo[4,5-b]pyridin-7-yl)azepan-1- yl]methanone, Isomer 1 |
| 275 | 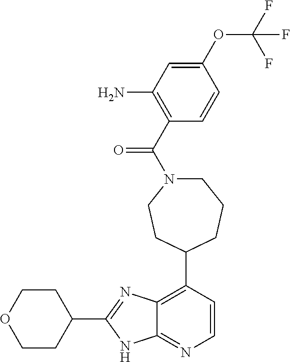 [SMILES: Nc1cc(OC(F)(F)F)ccc1C(=O)N1CCCC(c2ccnc3[nH]c(C4CCOCC4)nc23)CC1] Isomer 2 | [2-amino-4- (trifluoromethoxy)phenyl]-[4-(2- tetrahydropyran-4-yl-3H- imidazo[4,5-b]pyridin-7-yl)azepan-1- yl]methanone, Isomer 2 |
| 276 | 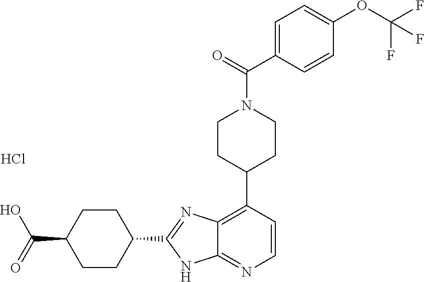 [SMILES: Cl ; O=C(c1ccc(OC(F)(F)F)cc1)N1CCC(c2ccnc3[nH]c([C@H]4CC[C@H](C(=O)O)CC4)nc23)CC1] | (trans)-4-[7-[1-[4- (trifluoromethoxy)benzoyl]-4- piperidyl]-3H-imidazo[4,5-b]pyridin- 2-yl]cyclohexanecarboxylic acid;hydrochloride |
| 277 | 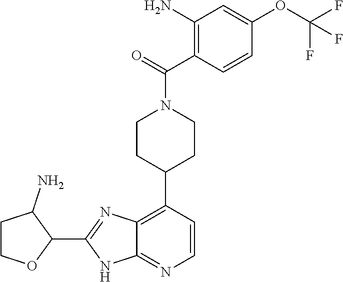 [SMILES: Nc1cc(OC(F)(F)F)ccc1C(=O)N1CCC(c2ccnc3[nH]c(C4OCCC4N)nc23)CC1] Isomer 1 | (cis)-[4-[2-[3-aminotetrahydrofuran- 2-yl]-3H-imidazo[4,5-b]pyridin-7- yl]-1-piperidyl]-[2-amino-4- (trifluoromethoxy)phenyl]methanone, Isomer 1 |
| 278 | 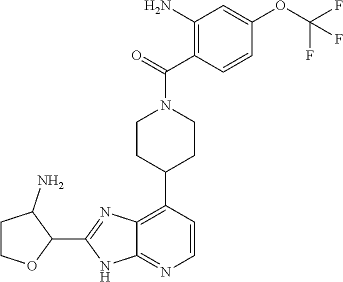 [SMILES: Nc1cc(OC(F)(F)F)ccc1C(=O)N1CCC(c2ccnc3[nH]c(C4OCCC4N)nc23)CC1] Isomer 2 | (cis)-[4-[2-[3-aminotetrahydrofuran- 2-yl]-3H-imidazo[4,5-b]pyridin-7- yl]-1-piperidyl]-[2-amino-4- (trifluoromethoxy)phenyl]methanone, Isomer 2 |
| 279 | 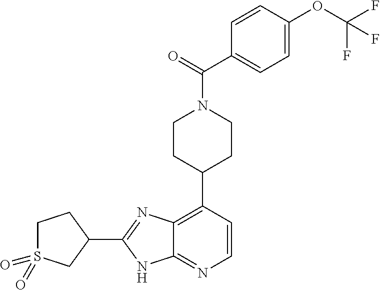 [SMILES: O=C(c1ccc(OC(F)(F)F)cc1)N1CCC(c2ccnc3[nH]c(C4CCS(=O)(=O)C4)nc23)CC1] | (rac)-[4-[2-(1,1-dioxothiolan-3-yl)- 3H-imidazo[4,5-b]pyridin-7-yl]-1- piperidyl]-[4- (trifluoromethoxy)phenyl]methanone |
| 280 | 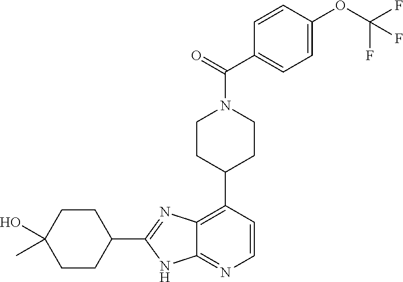 [SMILES: CC1(O)CCC(c2nc3c(C4CCN(C(=O)c5ccc(OC(F)(F)F)cc5)CC4)ccnc3[nH]2)CC1] | [4-[2-(4-hydroxy-4-methyl- cyclohexyl)-3H-imidazo[4,5- b]pyridin-7-yl]-1-piperidyl]-[4- (trifluoromethoxy)phenyl]methanone |
| 281 | 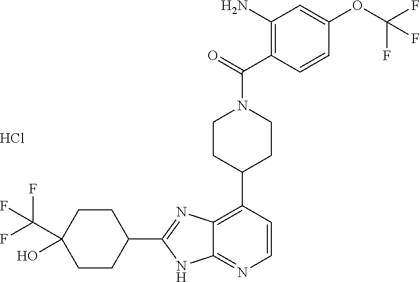 [SMILES: Nc1cc(OC(F)(F)F)ccc1C(=O)N1CCC(c2ccnc3[nH]c(C4CCC(O)(C(F)(F)F)CC4)nc23)CC1 ; Cl] | [2-amino-4- (trifluoromethoxy)phenyl]-[4-[2-[4- hydroxy-4- (trifluoromethyl)cyclohexyl]-3H- imidazo[4,5-b]pyridin-7-yl]-1- piperidyl]methanone;hydrochloride |
| 282 | 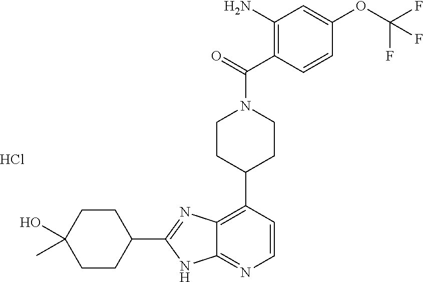 [SMILES: Cl ; CC1(O)CCC(c2nc3c(C4CCN(C(=O)c5ccc(OC(F)(F)F)cc5N)CC4)ccnc3[nH]2)CC1] | [2-amino-4- (trifluoromethoxy)phenyl]-[4-[2-(4- hydroxy-4-methyl-cyclohexyl)-3H- imidazo[4,5-b]pyridin-7-yl]-1- piperidyl]methanone;hydrochloride |
| 283 | 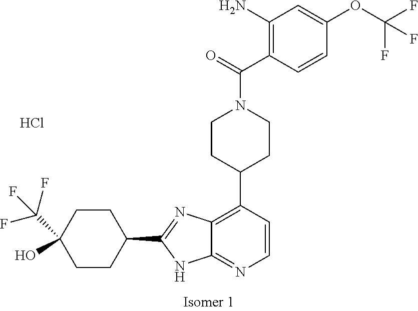 [SMILES: Nc1cc(OC(F)(F)F)ccc1C(=O)N1CCC(c2ccnc3[nH]c([C@H]4CC[C@](O)(C(F)(F)F)CC4)nc23)CC1 ; Cl] | [2-amino-4- (trifluoromethoxy)phenyl]-[4-[2-[4- hydroxy-4- (trifluoromethyl)cyclohexyl]-3H- imidazo[4,5-b]pyridin-7-yl]-1- piperidyl]methanone;hydrochloride, Isomer 1 |
| 284 | 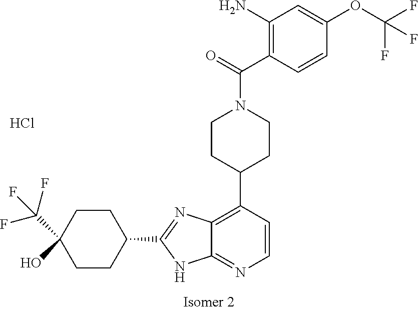 [SMILES: Nc1cc(OC(F)(F)F)ccc1C(=O)N1CCC(c2ccnc3[nH]c([C@H]4CC[C@@](O)(C(F)(F)F)CC4)nc23)CC1 ; Cl] | [2-amino-4- (trifluoromethoxy)phenyl]-[4-[2-[4- hydroxy-4- (trifluoromethyl)cyclohexyl]-3H- imidazo[4,5-b]pyridin-7-yl]-1- piperidyl]methanone;hydrochloride, Isomer 2 |
| 285 | 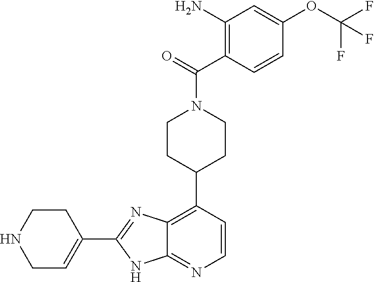 [SMILES: Nc1cc(OC(F)(F)F)ccc1C(=O)N1CCC(c2ccnc3[nH]c(C4=CCNCC4)nc23)CC1] | [2-amino-4- (trifluoromethoxy)phenyl]-[4-[2- (1,2,3,6-tetrahydropyridin-4-yl)-3H- imidazo[4,5-b]pyridin-7-yl]-1- piperidyl]methanone |
| 286 | 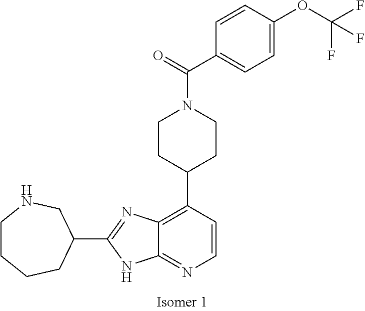 [SMILES: O=C(c1ccc(OC(F)(F)F)cc1)N1CCC(c2ccnc3[nH]c(C4CCCCNC4)nc23)CC1] | [4-[2-[azepan-3-yl]-3H-imidazo[4,5- b]pyridin-7-yl]-1-piperidyl]-[4- (trifluoromethoxy)phenyl]methanone, Isomer 1 |
| 287 | 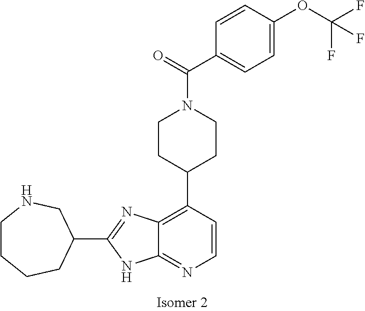 [SMILES: O=C(c1ccc(OC(F)(F)F)cc1)N1CCC(c2ccnc3[nH]c(C4CCCCNC4)nc23)CC1] | [4-[2-[azepan-3-yl]-3H-imidazo[4,5- b]pyridin-7-yl]-1-piperidyl]-[4- (trifluoromethoxy)phenyl]methanone, Isomer 2 |
| 288 | 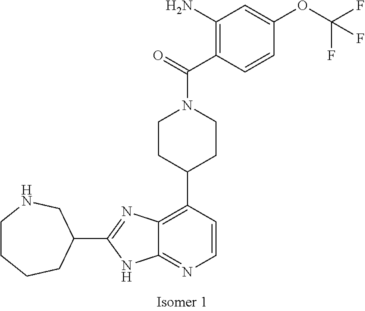 [SMILES: Nc1cc(OC(F)(F)F)ccc1C(=O)N1CCC(c2ccnc3[nH]c(C4CCCCNC4)nc23)CC1] | [2-amino-4- (trifluoromethoxy)phenyl]-[4-[2- [azepan-3-yl]-3H-imidazo[4,5- b]pyridin-7-yl]-1- piperidyl]methanone, Isomer 1 |
| 289 | 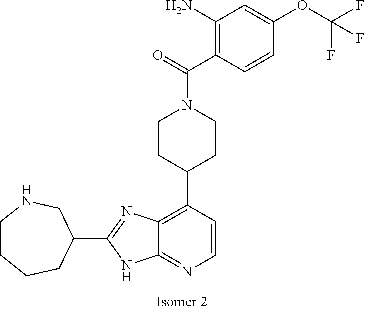 [SMILES: Nc1cc(OC(F)(F)F)ccc1C(=O)N1CCC(c2ccnc3[nH]c(C4CCCCNC4)nc23)CC1] | [2-amino-4- (trifluoromethoxy)phenyl]-[4-[2- [azepan-3-yl]-3H-imidazo[4,5- b]pyridin-7-yl]-1- piperidyl]methanone, Isomer 2 |
| 290 | 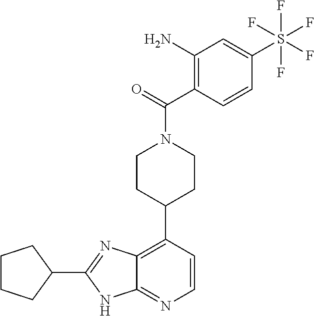 [SMILES: Nc1cc(S(F)(F)(F)(F)F)ccc1C(=O)N1CCC(c2ccnc3[nH]c(C4CCCC4)nc23)CC1] | [2-amino-4-(pentafluoro-λ⁶- sulfanyl)phenyl]-[4-(2-cyclopentyl- 3H-imidazo[4,5-b]pyridin-7-yl)-1- piperidyl]methanone |
| 291 | 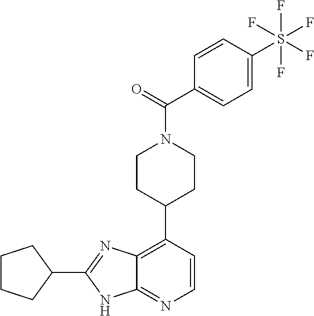 [SMILES: O=C(c1ccc(S(F)(F)(F)(F)F)cc1)N1CCC(c2ccnc3[nH]c(C4CCCC4)nc23)CC1] | [4-(2-cyclopentyl-3H-imidazo[4,5- b]pyridin-7-yl)-1-piperidyl]-[4- (pentafluoro-λ⁶- sulfanyl)phenyl]methanone |
| 292 | 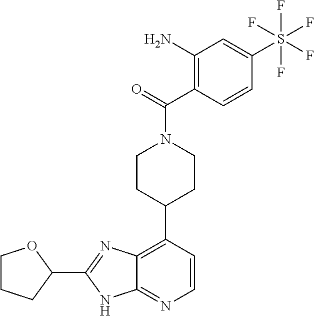 [SMILES: Nc1cc(S(F)(F)(F)(F)F)ccc1C(=O)N1CCC(c2ccnc3[nH]c(C4CCCO4)nc23)CC1] | (rac)-[2-amino-4-(pentafluoro-λ⁶- sulfanyl)phenyl]-[4-(2- tetrahydrofuran-2-yl-3H- imidazo[4,5-b]pyridin-7-yl)-1- piperidyl]methanone |
| 293 | 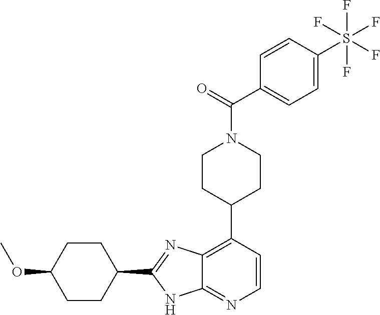 [SMILES: CO[C@H]1CC[C@@H](c2nc3c(C4CCN(C(=O)c5ccc(S(F)(F)(F)(F)F)cc5)CC4)ccnc3[nH]2)CC1] | (cis)-[4-[2-(4-methoxycyclohexyl)- 3H-imidazo[4,5-b]pyridin-7-yl]-1- piperidyl]-[4-(pentafluoro-λ⁶- sulfanyl)phenyl]methanone |
| 294 | 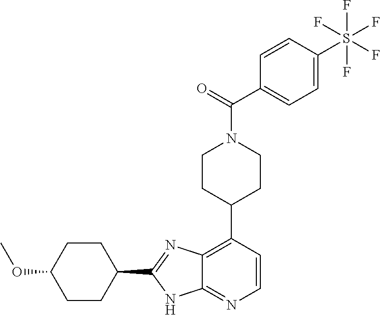 [SMILES: CO[C@H]1CC[C@H](c2nc3c(C4CCN(C(=O)c5ccc(S(F)(F)(F)(F)F)cc5)CC4)ccnc3[nH]2)CC1] | (trans)-[4-[2-(4-methoxycyclohexyl)- 3H-imidazo[4,5-b]pyridin-7-yl]-1- piperidyl]-[4-(pentafluoro-λ⁶- sulfanyl)phenyl]methanone |
| 295 | 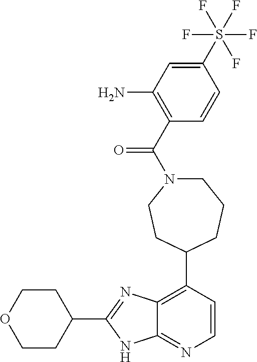 [SMILES: Nc1cc(S(F)(F)(F)(F)F)ccc1C(=O)N1CCCC(c2ccnc3[nH]c(C4CCOCC4)nc23)CC1] | (rac)-[2-amino-4-(pentafluoro-λ⁶- sulfanyl)phenyl]-[4-(2- tetrahydropyran-4-yl-3H- imidazo[4,5-b]pyridin-7-yl)azepan-1- yl]methanone |
| 296 | 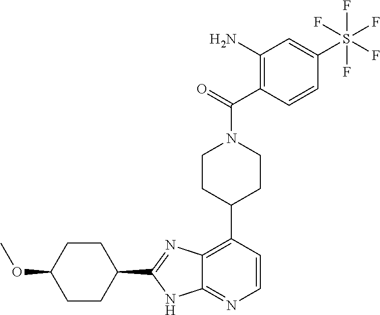 [SMILES: CO[C@H]1CC[C@@H](c2nc3c(C4CCN(C(=O)c5ccc(S(F)(F)(F)(F)F)cc5N)CC4)ccnc3[nH]2)CC1] | (cis)-[2-amino-4-(pentafluoro-λ⁶- sulfanyl)phenyl]-[4-[2-(4- methoxycyclohexyl)-3H- imidazo[4,5-b]pyridin-7-yl]-1- piperidyl]methanone |
| 297 | 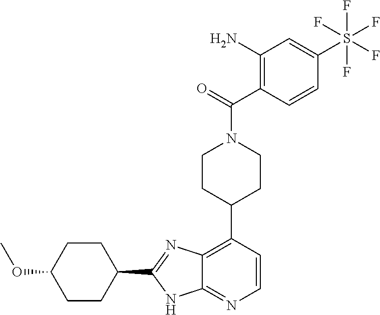 [SMILES: CO[C@H]1CC[C@H](c2nc3c(C4CCN(C(=O)c5ccc(S(F)(F)(F)(F)F)cc5N)CC4)ccnc3[nH]2)CC1] | (trans)-[2-amino-4-(pentafluoro-λ⁶- sulfanyl)phenyl]-[4-[2-(4- methoxycyclohexyl)-3H- imidazo[4,5-b]pyridin-7-yl]-1- piperidyl]methanone |
| 298 | 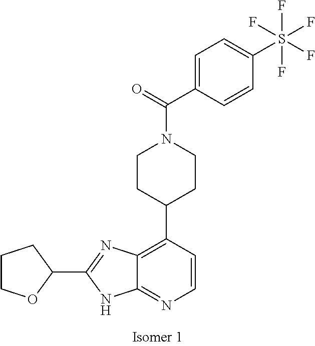 [SMILES: O=C(c1ccc(S(F)(F)(F)(F)F)cc1)N1CCC(c2ccnc3[nH]c(C4CCCO4)nc23)CC1] | [4-(pentafluoro-λ⁶-sulfanyl)phenyl]- [4-[2-[tetrahydrofuran-2-yl]-3H- imidazo[4,5-b]pyridin-7-yl]-1- piperidyl]methanone, Isomer 1 |
| 299 | 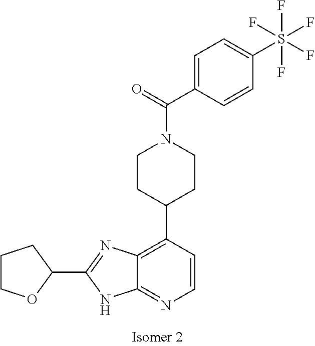 [SMILES: O=C(c1ccc(S(F)(F)(F)(F)F)cc1)N1CCC(c2ccnc3[nH]c(C4CCCO4)nc23)CC1] | [4-(pentafluoro-λ⁶-sulfanyl)phenyl]- [4-[2-[tetrahydrofuran-2-yl]-3H- imidazo[4,5-b]pyridin-7-yl]-1- piperidyl]methanone, Isomer 2 |
| 300 | 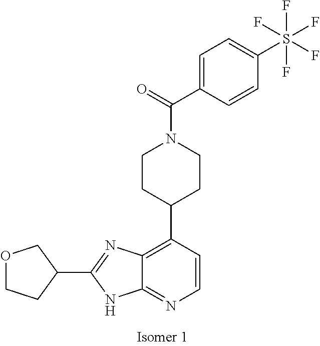 [SMILES: O=C(c1ccc(S(F)(F)(F)(F)F)cc1)N1CCC(c2ccnc3[nH]c(C4CCOC4)nc23)CC1] | [4-(pentafluoro-λ⁶-sulfanyl)phenyl]- [4-[2-[tetrahydrofuran-3-yl]-3H- imidazo[4,5-b]pyridin-7-yl]-1- piperidyl]methanone, Isomer 1 |
| 301 | 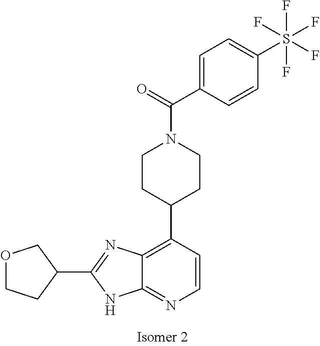 [SMILES: O=C(c1ccc(S(F)(F)(F)(F)F)cc1)N1CCC(c2ccnc3[nH]c(C4CCOC4)nc23)CC1] | [4-(pentafluoro-λ⁶-sulfanyl)phenyl]- [4-[2-[tetrahydrofuran-3-yl]-3H- imidazo[4,5-b]pyridin-7-yl]-1- piperidyl]methanone, Isomer 2 |
| 302 | 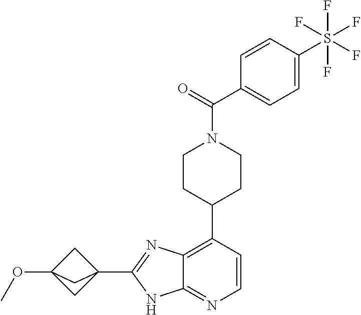 [SMILES: COC12CC(c3nc4c(C5CCN(C(=O)c6ccc(S(F)(F)(F)(F)F)cc6)CC5)ccnc4[nH]3)(C1)C2] | [4-[2-(3-methoxy-1- bicyclo[1.1.1]pentanyl)-3H- imidazo[4,5-b]pyridin-7-yl]-1- piperidyl]-[4-(pentafluoro-λ⁶- sulfanyl)phenyl]methanone |
| 303 | 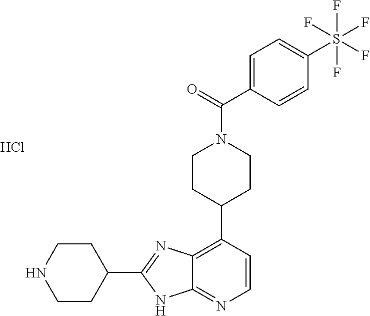 [SMILES: Cl ; O=C(c1ccc(S(F)(F)(F)(F)F)cc1)N1CCC(c2ccnc3[nH]c(C4CCNCC4)nc23)CC1] | [4-(pentafluoro-λ⁶-sulfanyl)phenyl]- [4-[2-(4-piperidyl)-3H-imidazo[4,5- b]pyridin-7-yl]-1- piperidyl]methanone;hydrochloride |
| 304 | 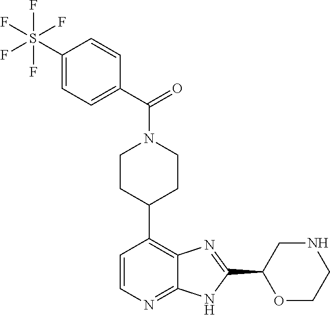 [SMILES: O=C(c1ccc(S(F)(F)(F)(F)F)cc1)N1CCC(c2ccnc3[nH]c([C@H]4CNCCO4)nc23)CC1] | [4-[2-[(2R)-morpholin-2-yl]-3H- imidazo[4,5-b]pyridin-7-yl]-1- piperidyl]-[4-(pentafluoro-λ⁶- sulfanyl)phenyl]methanone |
| 305 | 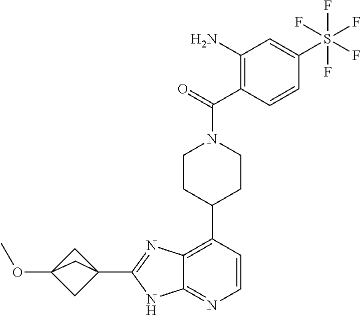 [SMILES: COC12CC(c3nc4c(C5CCN(C(=O)c6ccc(S(F)(F)(F)(F)F)cc6N)CC5)ccnc4[nH]3)(C1)C2] | [2-amino-4-(pentafluoro-λ⁶- sulfanyl)phenyl]-[4-[2-(3-methoxy-1- bicyclo[1.1.1]pentanyl)-3H- imidazo[4,5-b]pyridin-7-yl]-1- piperidyl]methanone |
| 306 | 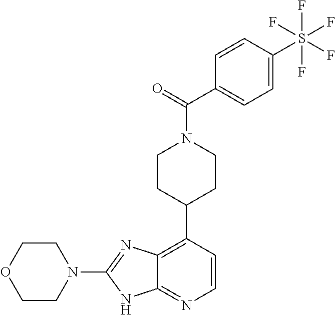 [SMILES: O=C(c1ccc(S(F)(F)(F)(F)F)cc1)N1CCC(c2ccnc3[nH]c(N4CCOCC4)nc23)CC1] | [4-(2-N-morpholino-3H-imidazo[4,5- b]pyridin-7-yl)-1-piperidyl]-[4- (pentafluoro-λ⁶- sulfanyl)phenyl]methanone |
| 307 | 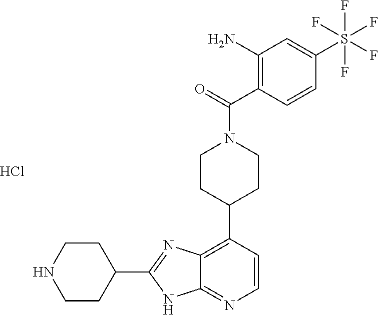 [SMILES: Cl ; Nc1cc(S(F)(F)(F)(F)F)ccc1C(=O)N1CCC(c2ccnc3[nH]c(C4CCNCC4)nc23)CC1] | [2-amino-4-(pentafluoro-λ⁶- sulfanyl)phenyl]-[4-[2-(4-piperidyl)- 3H-imidazo[4,5-b]pyridin-7-yl]-1- piperidyl]methanone;hydrochloride |
| 308 | 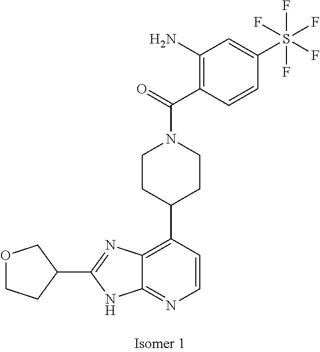 [SMILES: Nc1cc(S(F)(F)(F)(F)F)ccc1C(=O)N1CCC(c2ccnc3[nH]c(C4CCOC4)nc23)CC1] | [2-amino-4-(pentafluoro-λ⁶- sulfanyl)phenyl]-[4-[2- [tetrahydrofuran-3-yl]-3H- imidazo[4,5-b]pyridin-7-yl]-1- piperidyl]methanone, Isomer 1 |
| 309 | 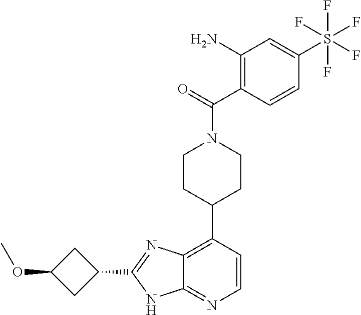 [SMILES: CO[C@H]1C[C@H](c2nc3c(C4CCN(C(=O)c5ccc(S(F)(F)(F)(F)F)cc5N)CC4)ccnc3[nH]2)C1] | (cis)-[2-amino-4-(pentafluoro-λ⁶- sulfanyl)phenyl]-[4-[2-(3- methoxycyclobutyl)-3H-imidazo[4,5- b]pyridin-7-yl]-1- piperidyl]methanone |
| 310 | 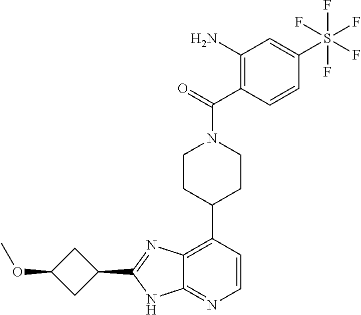 [SMILES: CO[C@H]1C[C@@H](c2nc3c(C4CCN(C(=O)c5ccc(S(F)(F)(F)(F)F)cc5N)CC4)ccnc3[nH]2)C1] | (trans)-[2-amino-4-(pentafluoro-λ⁶- sulfanyl)phenyl]-[4-[2-(3- methoxycyclobutyl)-3H-imidazo[4,5- b]pyridin-7-yl]-1- piperidyl]methanone |
| 311 | 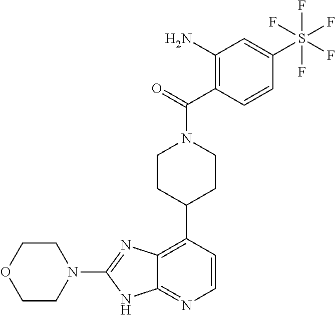 [SMILES: Nc1cc(S(F)(F)(F)(F)F)ccc1C(=O)N1CCC(c2ccnc3[nH]c(N4CCOCC4)nc23)CC1] | [2-amino-4-(pentafluoro-λ⁶- sulfanyl)phenyl]-[4-(2-N- morpholino-3H-imidazo[4,5- b]pyridin-7-yl)-1- piperidyl]methanone |
| 312 | 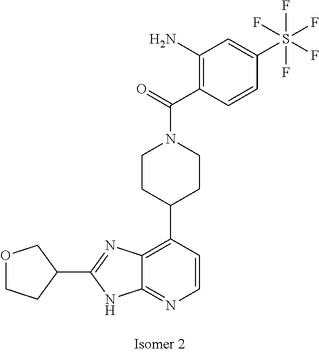 [SMILES: Nc1cc(S(F)(F)(F)(F)F)ccc1C(=O)N1CCC(c2ccnc3[nH]c(C4CCOC4)nc23)CC1] | [2-amino-4-(pentafluoro-λ⁶- sulfanyl)phenyl]-[4-[2- [tetrahydrofuran-3-yl]-3H- imidazo[4,5-b]pyridin-7-yl]-1- piperidyl]methanone, Isomer 2 |
| 313 | 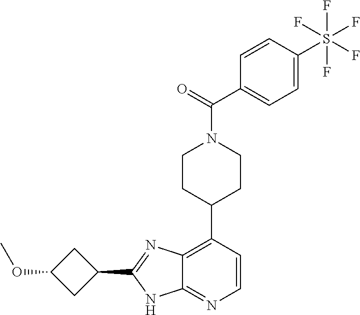 [SMILES: CO[C@H]1C[C@H](c2nc3c(C4CCN(C(=O)c5ccc(S(F)(F)(F)(F)F)cc5)CC4)ccnc3[nH]2)C1] | (trans)-[4-[2-(3-methoxycyclobutyl)- 3H-imidazo[4,5-b]pyridin-7-yl]-1- piperidyl]-[4-(pentafluoro-λ⁶- sulfanyl)phenyl]methanone |
| 314 | 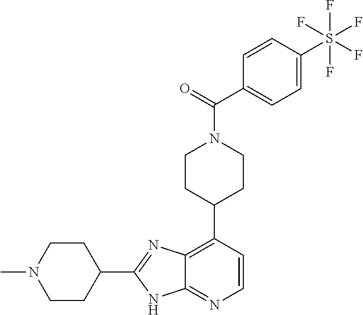 [SMILES: CN1CCC(c2nc3c(C4CCN(C(=O)c5ccc(S(F)(F)(F)(F)F)cc5)CC4)ccnc3[nH]2)CC1] | [4-[2-(1-methyl-4-piperidyl)-3H- imidazo[4,5-b]pyridin-7-yl]-1- piperidyl]-[4-(pentafluoro-λ⁶- sulfanyl)phenyl]methanone |
| 315 | 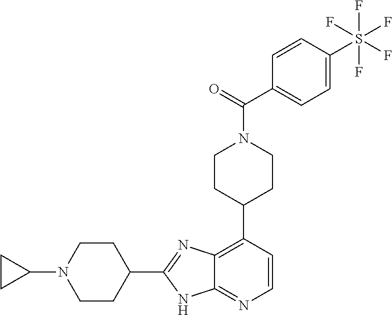 [SMILES: O=C(c1ccc(S(F)(F)(F)(F)F)cc1)N1CCC(c2ccnc3[nH]c(C4CCN(C5CC5)CC4)nc23)CC1] | [4-[2-(1-cyclopropyl-4-piperidyl)- 3H-imidazo[4,5-b]pyridin-7-yl]-1- piperidyl]-[4-(pentafluoro-λ⁶- sulfanyl)phenyl]methanone |
| 316 | 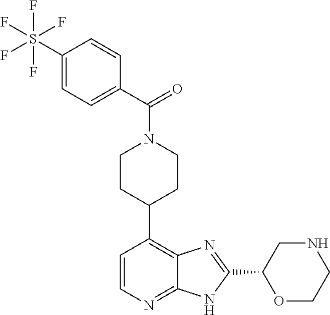 [SMILES: O=C(c1ccc(S(F)(F)(F)(F)F)cc1)N1CCC(c2ccnc3[nH]c([C@@H]4CNCCO4)nc23)CC1] | [4-[2-[(2S)-morpholin-2-yl]-3H- imidazo[4,5-b]pyridin-7-yl]-1- piperidyl]-[4-(pentafluoro-λ⁶- sulfanyl)phenyl]methanone |
| 317 | 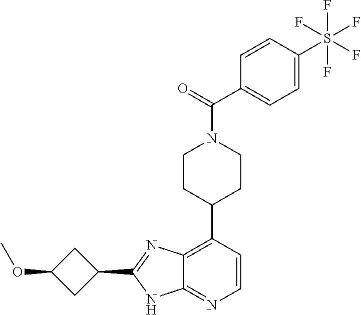 [SMILES: CO[C@H]1C[C@@H](c2nc3c(C4CCN(C(=O)c5ccc(S(F)(F)(F)(F)F)cc5)CC4)ccnc3[nH]2)C1] | (cis)-[4-[2-(3-methoxycyclobutyl)- 3H-imidazo[4,5-b]pyridin-7-yl]-1- piperidyl]-[4-(pentafluoro-λ⁶- sulfanyl)phenyl]methanone |
| 318 | 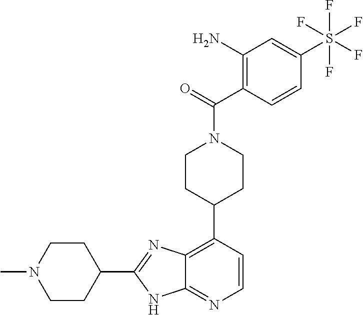 [SMILES: CN1CCC(c2nc3c(C4CCN(C(=O)c5ccc(S(F)(F)(F)(F)F)cc5N)CC4)ccnc3[nH]2)CC1] | [2-amino-4-(pentafluoro-λ⁶- sulfanyl)phenyl]-[4-[2-(1-methyl-4- piperidyl)-3H-imidazo[4,5-b]pyridin- 7-yl]-1-piperidyl]methanone |
| 319 | 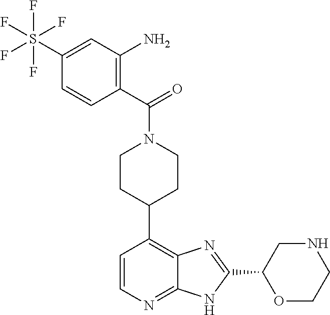 [SMILES: Nc1cc(S(F)(F)(F)(F)F)ccc1C(=O)N1CCC(c2ccnc3[nH]c([C@@H]4CNCCO4)nc23)CC1] | [2-amino-4-(pentafluoro-λ⁶- sulfanyl)phenyl]-[4-[2-[(2S)- morpholin-2-yl]-3H-imidazo[4,5- b]pyridin-7-yl]-1- piperidyl]methanone |
| 320 | 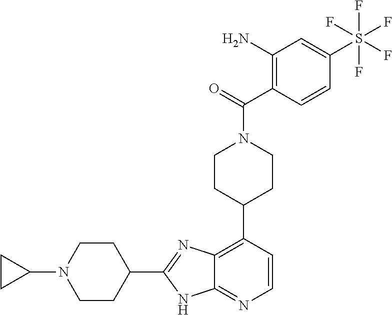 [SMILES: Nc1cc(S(F)(F)(F)(F)F)ccc1C(=O)N1CCC(c2ccnc3[nH]c(C4CCN(C5CC5)CC4)nc23)CC1] | [2-amino-4-(pentafluoro-λ⁶- sulfanyl)phenyl]-[4-[2-(1- cyclopropyl-4-piperidyl)-3H- imidazo[4,5-b]pyridin-7-yl]-1- piperidyl]methanone |
| 321 | 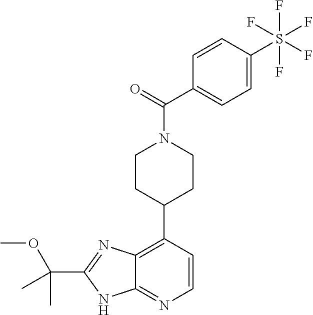 [SMILES: COC(C)(C)c1nc2c(C3CCN(C(=O)c4ccc(S(F)(F)(F)(F)F)cc4)CC3)ccnc2[nH]1] | [4-[2-(1-methoxy-1-methyl-ethyl)- 3H-imidazo[4,5-b]pyridin-7-yl]-1- piperidyl]-[4-(pentafluoro-λ⁶- sulfanyl)phenyl]methanone |
| 322 | 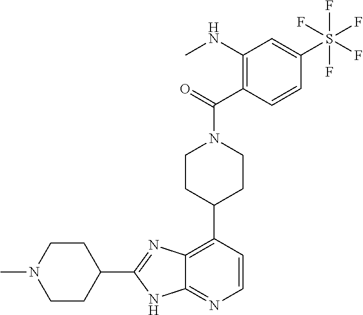 [SMILES: CNc1cc(S(F)(F)(F)(F)F)ccc1C(=O)N1CCC(c2ccnc3[nH]c(C4CCN(C)CC4)nc23)CC1] | [2-(methylamino)-4-(pentafluoro-λ⁶- sulfanyl)phenyl]-[4-[2-(1-methyl-4- piperidyl)-3H-imidazo[4,5-b]pyridin- 7-yl]-1-piperidyl]methanone |
| 323 | 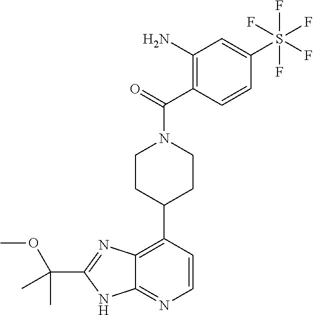 [SMILES: COC(C)(C)c1nc2c(C3CCN(C(=O)c4ccc(S(F)(F)(F)(F)F)cc4N)CC3)ccnc2[nH]1] | [2-amino-4-(pentafluoro-λ⁶- sulfanyl)phenyl]-[4-[2-(1-methoxy-1- methyl-ethyl)-3H-imidazo[4,5- b]pyridin-7-yl]-1- piperidyl]methanone |
| 324 | 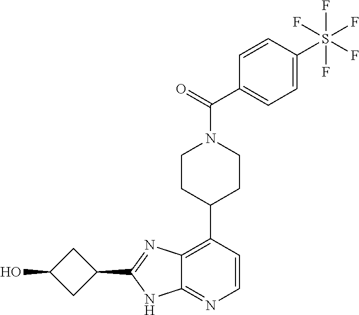 [SMILES: O=C(c1ccc(S(F)(F)(F)(F)F)cc1)N1CCC(c2ccnc3[nH]c([C@H]4C[C@@H](O)C4)nc23)CC1] | (cis)-[4-[2-(3-hydroxycyclobutyl)- 3H-imidazo[4,5-b]pyridin-7-yl]-1- piperidyl]-[4-(pentafluoro-λ⁶- sulfanyl)phenyl]methanone |
| 325 | 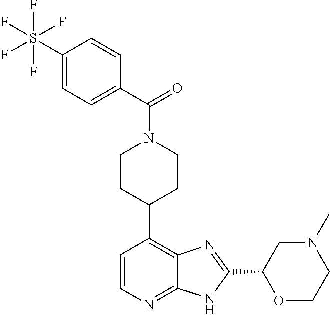 [SMILES: CN1CCO[C@H](c2nc3c(C4CCN(C(=O)c5ccc(S(F)(F)(F)(F)F)cc5)CC4)ccnc3[nH]2)C1] | [4-[2-[(2S)-4-methylmorpholin-2-yl]- 3H-imidazo[4,5-b]pyridin-7-yl]-1- piperidyl]-[4-(pentafluoro-λ⁶- sulfanyl)phenyl]methanone |
| 326 | 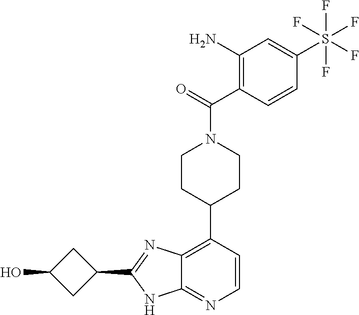 [SMILES: Nc1cc(S(F)(F)(F)(F)F)ccc1C(=O)N1CCC(c2ccnc3[nH]c([C@H]4C[C@@H](O)C4)nc23)CC1] | (cis)-[2-amino-4-(pentafluoro-λ⁶- sulfanyl)phenyl]-[4-[2-(3- hydroxycyclobutyl)-3H-imidazo[4,5- b]pyridin-7-yl]-1- piperidyl]methanone |
| 327 | 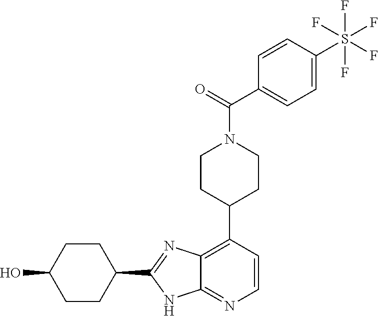 [SMILES: O=C(c1ccc(S(F)(F)(F)(F)F)cc1)N1CCC(c2ccnc3[nH]c([C@H]4CC[C@@H](O)CC4)nc23)CC1] | (cis)-[4-[2-(4-hydroxycyclohexyl)- 3H-imidazo[4,5-b]pyridin-7-yl]-1- piperidyl]-[4-(pentafluoro-λ⁶- sulfanyl)phenyl]methanone |
| 328 | 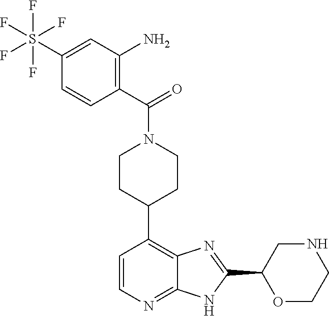 [SMILES: Nc1cc(S(F)(F)(F)(F)F)ccc1C(=O)N1CCC(c2ccnc3[nH]c([C@H]4CNCCO4)nc23)CC1] | [2-amino-4-(pentafluoro-λ⁶- sulfanyl)phenyl]-[4-[2-[(2R)- morpholin-2-yl]-3H-imidazo[4,5- b]pyridin-7-yl]-1- |
| 329 | 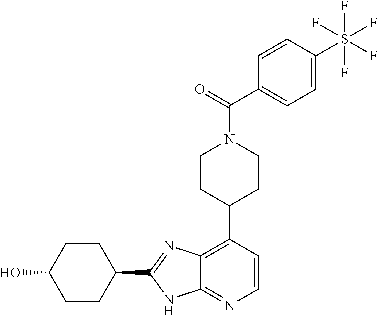 [SMILES: O=C(c1ccc(S(F)(F)(F)(F)F)cc1)N1CCC(c2ccnc3[nH]c([C@H]4CC[C@H](O)CC4)nc23)CC1] | (trans)-[4-[2-(4-hydroxycyclohexyl)- 3H-imidazo[4,5-b]pyridin-7-yl]-1- piperidyl]-[4-(pentafluoro-λ⁶- sulfanyl)phenyl]methanone |
| 330 | 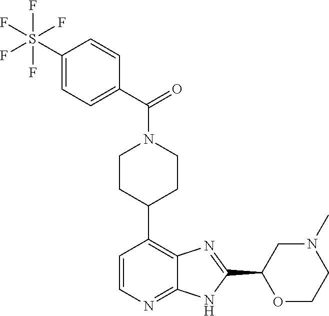 [SMILES: CN1CCO[C@@H](c2nc3c(C4CCN(C(=O)c5ccc(S(F)(F)(F)(F)F)cc5)CC4)ccnc3[nH]2)C1] | [4-[2-[(2R)-4-methylmorpholin-2- yl]-3H-imidazo[4,5-b]pyridin-7-yl]- 1-piperidyl]-[4-(pentafluoro-λ⁶- sulfanyl)phenyl]methanone |
| 331 | 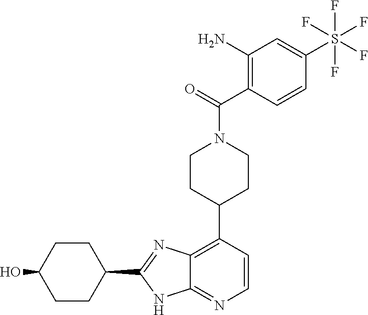 [SMILES: Nc1cc(S(F)(F)(F)(F)F)ccc1C(=O)N1CCC(c2ccnc3[nH]c([C@H]4CC[C@@H](O)CC4)nc23)CC1] | (cis)-[2-amino-4-(pentafluoro-λ⁶- sulfanyl)phenyl]-[4-[2-(4- hydroxycyclohexyl)-3H-imidazo[4,5- b]pyridin-7-yl]-1- piperidyl]methanone |
| 332 | 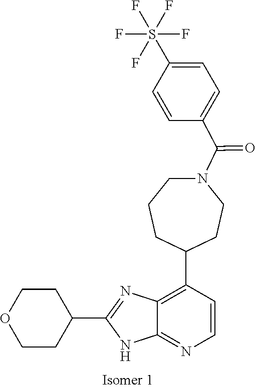 [SMILES: O=C(c1ccc(S(F)(F)(F)(F)F)cc1)N1CCCC(c2ccnc3[nH]c(C4CCOCC4)nc23)CC1] | [4-(pentafluoro-λ⁶-sulfanyl)phenyl]- [4-(2-tetrahydropyran-4-yl-3H- imidazo[4,5-b]pyridin-7-yl)azepan-1- yl]methanone, Isomer 1 |
| 333 | 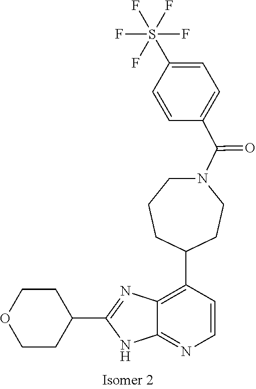 [SMILES: O=C(c1ccc(S(F)(F)(F)(F)F)cc1)N1CCCC(c2ccnc3[nH]c(C4CCOCC4)nc23)CC1] | [4-(pentafluoro-λ⁶-sulfanyl)phenyl]- [4-(2-tetrahydropyran-4-yl-3H- imidazo[4,5-b]pyridin-7-yl)azepan-1- yl]methanone, Isomer 2 |
| 334 | 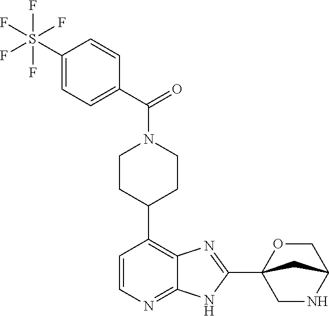 [SMILES: O=C(c1ccc(S(F)(F)(F)(F)F)cc1)N1CCC(c2ccnc3[nH]c([C@@]45CN[C@@H](CO4)C5)nc23)CC1] | [4-[2-[(1R,4R)-2-oxa-5- azabicyclo[2.2.1]heptan-1-yl]-3H- imidazo[4,5-b]pyridin-7-yl]-1- piperidyl]-[4-(pentafluoro-λ⁶- sulfanyl)phenyl]methanone |
| 335 | 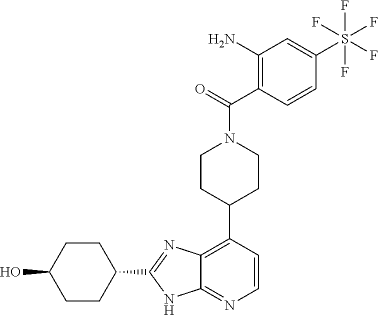 [SMILES: Nc1cc(S(F)(F)(F)(F)F)ccc1C(=O)N1CCC(c2ccnc3[nH]c([C@H]4CC[C@H](O)CC4)nc23)CC1] | (trans)-[2-amino-4-(pentafluoro-λ⁶- sulfanyl)phenyl]-[4-[2-(4- hydroxycyclohexyl)-3H-imidazo[4,5- b]pyridin-7-yl]-1- piperidyl]methanone |
| 336 | 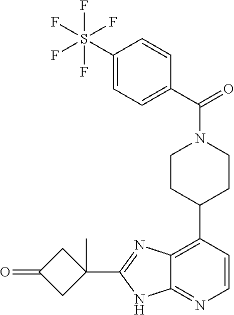 [SMILES: CC1(c2nc3c(C4CCN(C(=O)c5ccc(S(F)(F)(F)(F)F)cc5)CC4)ccnc3[nH]2)CC(=O)C1] | 3-methyl-3-[7-[1-[4-(pentafluoro-λ⁶- sulfanyl)benzoyl]-4-piperidyl]-3H- imidazo[4,5-b]pyridin-2- yl]cyclobutanone |
| 337 | 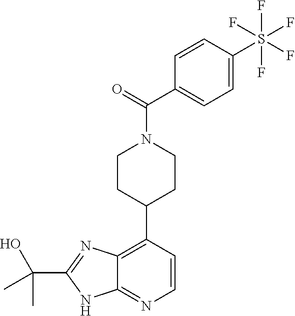 [SMILES: CC(C)(O)c1nc2c(C3CCN(C(=O)c4ccc(S(F)(F)(F)(F)F)cc4)CC3)ccnc2[nH]1] | [4-[2-(1-hydroxy-1-methyl-ethyl)- 3H-imidazo[4,5-b]pyridin-7-yl]-1- piperidyl]-[4-(pentafluoro-λ⁶- sulfanyl)phenyl]methanone |
| 338 | 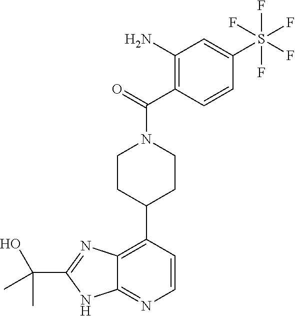 [SMILES: CC(C)(O)c1nc2c(C3CCN(C(=O)c4ccc(S(F)(F)(F)(F)F)cc4N)CC3)ccnc2[nH]1] | [2-amino-4-(pentafluoro-λ⁶- sulfanyl)phenyl]-[4-[2-(1-hydroxy-1- methyl-ethyl)-3H-imidazo[4,5- b]pyridin-7-yl]-1- piperidyl]methanone |
| 339 | 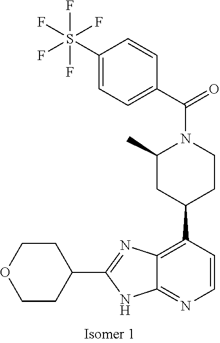 [SMILES: C[C@@H]1C[C@H](c2ccnc3[nH]c(C4CCOCC4)nc23)CCN1C(=O)c1ccc(S(F)(F)(F)(F)F)cc1] | [(cis)-2-methyl-4-(2-tetrahydropyran- 4-yl-3H-imidazo[4,5-b]pyridin-7-yl)- 1-piperidyl]-[4-(pentafluoro-λ⁶- sulfanyl)phenyl]methanone, Isomer 1 |
| 340 | 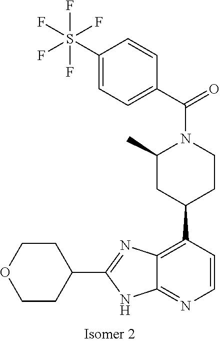 [SMILES: C[C@@H]1C[C@H](c2ccnc3[nH]c(C4CCOCC4)nc23)CCN1C(=O)c1ccc(S(F)(F)(F)(F)F)cc1] | [(cis)-2-methyl-4-(2- tetrahydropyran-4-yl-3H- imidazo[4,5-b]pyridin-7-yl)-1- piperidyl]-[4-(pentafluoro-λ⁶- sulfanyl)phenyl]methanone, Isomer 2 |
| 341 | 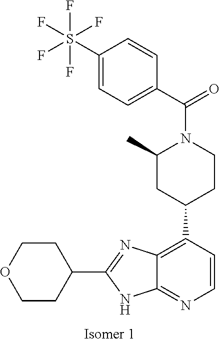 [SMILES: C[C@@H]1C[C@@H](c2ccnc3[nH]c(C4CCOCC4)nc23)CCN1C(=O)c1ccc(S(F)(F)(F)(F)F)cc1] | [(trans)-2-methyl-4-(2- tetrahydropyran-4-yl-3H- imidazo[4,5-b]pyridin-7-yl)-1- piperidyl]-[4-(pentafluoro-λ⁶- sulfanyl)phenyl]methanone, Isomer 1 |
| 342 | 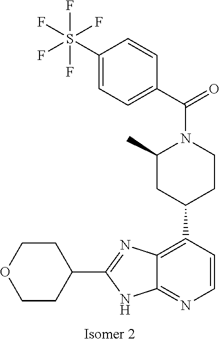 [SMILES: C[C@@H]1C[C@@H](c2ccnc3[nH]c(C4CCOCC4)nc23)CCN1C(=O)c1ccc(S(F)(F)(F)(F)F)cc1] | [(trans)-2-methyl-4-(2- tetrahydropyran-4-yl-3H- imidazo[4,5-b]pyridin-7-yl)-1- piperidyl]-[4-(pentafluoro-λ⁶- sulfanyl)phenyl]methanone, Isomer 2 |
| 343 | 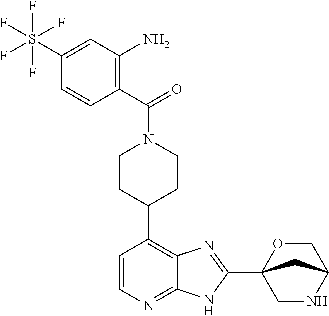 [SMILES: Nc1cc(S(F)(F)(F)(F)F)ccc1C(=O)N1CCC(c2ccnc3[nH]c([C@@]45CN[C@@H](CO4)C5)nc23)CC1] | [2-amino-4-(pentafluoro-λ⁶- sulfanyl)phenyl]-[4-[2-[(1R,4R)-2- oxa-5-azabicyclo[2.2.1]heptan-1-yl]- 3H-imidazo[4,5-b]pyridin-7-yl]-1- piperidyl]methanone |
| 344 | 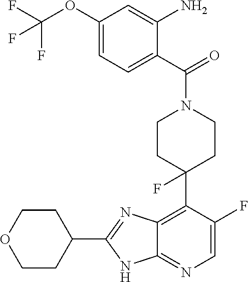 [SMILES: Nc1cc(OC(F)(F)F)ccc1C(=O)N1CCC(F)(c2c(F)cnc3[nH]c(C4CCOCC4)nc23)CC1] | [2-amino-4- (trifluoromethoxy)phenyl]-[4-fluoro- 4-(6-fluoro-2-tetrahydropyran-4-yl- 3H-imidazo[4,5-b]pyridin-7-yl)-1- piperidyl]methanone |
| 345 | 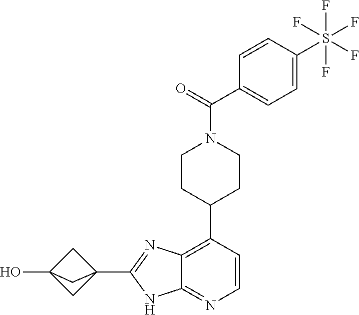 [SMILES: O=C(c1ccc(S(F)(F)(F)(F)F)cc1)N1CCC(c2ccnc3[nH]c(C45CC(O)(C4)C5)nc23)CC1] | [4-[2-(3-hydroxy-1- bicyclo[1.1.1]pentanyl)-3H- imidazo[4,5-b]pyridin-7-yl]-1- piperidyl]-[4-(pentafluoro-λ⁶- sulfanyl)phenyl]methanone |
| 346 | 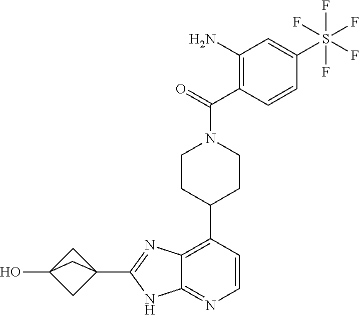 [SMILES: Nc1cc(S(F)(F)(F)(F)F)ccc1C(=O)N1CCC(c2ccnc3[nH]c(C45CC(O)(C4)C5)nc23)CC1] | [2-amino-4-(pentafluoro-λ⁶- sulfanyl)phenyl]-[4-[2-(3-hydroxy-1- bicyclo[1.1.1]pentanyl)-3H- imidazo[4,5-b]pyridin-7-yl]-1- piperidyl]methanone |
| 347 | 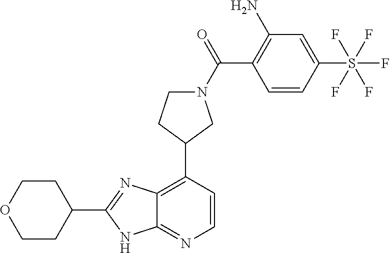 [SMILES: Nc1cc(S(F)(F)(F)(F)F)ccc1C(=O)N1CCC(c2ccnc3[nH]c(C4CCOCC4)nc23)C1] | (rac)-[2-amino-4-(pentafluoro-λ⁶- sulfanyl)phenyl]-[3-(2- tetrahydropyran-4-yl-3H- imidazo[4,5-b]pyridin-7- yl)pyrrolidin-1-yl]methanone |
| 348 | 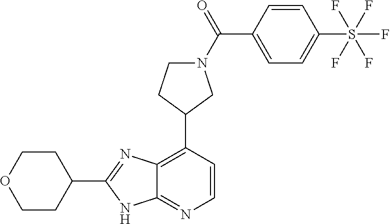 [SMILES: O=C(c1ccc(S(F)(F)(F)(F)F)cc1)N1CCC(c2ccnc3[nH]c(C4CCOCC4)nc23)C1] | (rac)-[4-(pentafluoro-λ⁶- sulfanyl)phenyl]-[3-(2- tetrahydropyran-4-yl-3H- imidazo[4,5-b]pyridin-7- yl)pyrrolidin-1-yl]methanone |
| 349 | 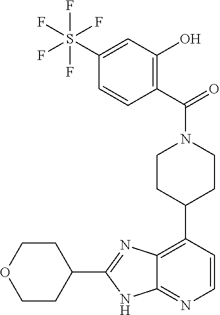 [SMILES: O=C(c1ccc(S(F)(F)(F)(F)F)cc1O)N1CCC(c2ccnc3[nH]c(C4CCOCC4)nc23)CC1] | [2-hydroxy-4-(pentafluoro-λ⁶- sulfanyl)phenyl]-[4-(2- tetrahydropyran-4-yl-3H- imidazo[4,5-b]pyridin-7-yl)-1- piperidyl]methanone |
| 350 | 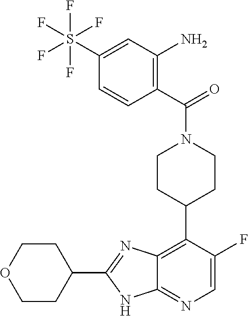 [SMILES: Nc1cc(S(F)(F)(F)(F)F)ccc1C(=O)N1CCC(c2c(F)cnc3[nH]c(C4CCOCC4)nc23)CC1] | [2-amino-4-(pentafluoro-λ⁶- sulfanyl)phenyl]-[4-(6-fluoro-2- tetrahydropyran-4-yl-3H- imidazo[4,5-b]pyridin-7-yl)-1- piperidyl]methanone |
| 351 | 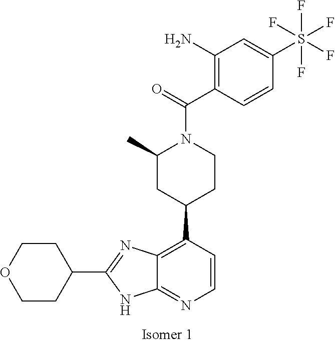 [SMILES: C[C@@H]1C[C@H](c2ccnc3[nH]c(C4CCOCC4)nc23)CCN1C(=O)c1ccc(S(F)(F)(F)(F)F)cc1N] | [2-amino-4-(pentafluoro-λ⁶- sulfanyl)phenyl]-[(cis)-2-methyl-4- (2-tetrahydropyran-4-yl-3H- imidazo[4,5-b]pyridin-7-yl)-1- piperidyl]methanone, Isomer 1 |
| 352 | 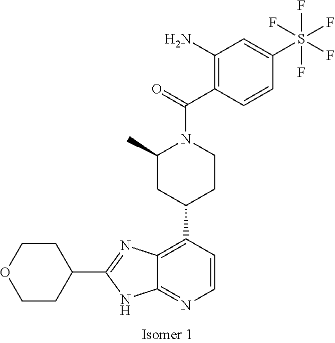 [SMILES: C[C@@H]1C[C@@H](c2ccnc3[nH]c(C4CCOCC4)nc23)CCN1C(=O)c1ccc(S(F)(F)(F)(F)F)cc1N] | [2-amino-4-(pentafluoro-λ⁶- sulfanyl)phenyl]-[(trans)-2-methyl-4- (2-tetrahydropyran-4-yl-3H- imidazo[4,5-b]pyridin-7-yl)-1- piperidyl]methanone, Isomer 1 |
| 353 | 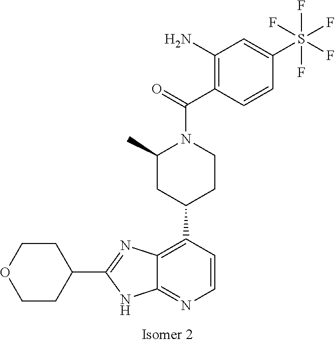 [SMILES: C[C@@H]1C[C@@H](c2ccnc3[nH]c(C4CCOCC4)nc23)CCN1C(=O)c1ccc(S(F)(F)(F)(F)F)cc1N] | [2-amino-4-(pentafluoro-λ⁶- sulfanyl)phenyl]-[(trans)-2-methyl-4- (2-tetrahydropyran-4-yl-3H- imidazo[4,5-b]pyridin-7-yl)-1- piperidyl]methanone, Isomer 2 |
| 354 | 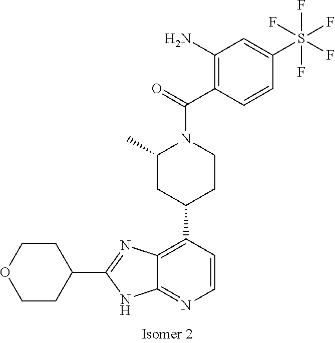 [SMILES: C[C@H]1C[C@@H](c2ccnc3[nH]c(C4CCOCC4)nc23)CCN1C(=O)c1ccc(S(F)(F)(F)(F)F)cc1N] | [2-amino-4-(pentafluoro-λ⁶- sulfanyl)phenyl]-[(cis)-2-methyl-4- (2-tetrahydropyran-4-yl-3H- imidazo[4,5-b]pyridin-7-yl)-1- piperidyl]methanone, Isomer 2 |
| 355 | 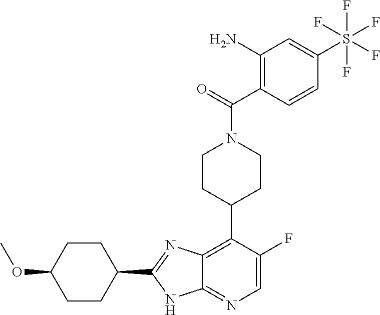 [SMILES: CO[C@H]1CC[C@@H](c2nc3c(C4CCN(C(=O)c5ccc(S(F)(F)(F)(F)F)cc5N)CC4)c(F)cnc3[nH]2)CC1] | (cis)-[2-amino-4-(pentafluoro-λ⁶- sulfanyl)phenyl]-[4-[6-fluoro-2-(4- methoxycyclohexyl)-3H- imidazo[4,5-b]pyridin-7-yl]-1- piperidyl]methanone |
| 356 | 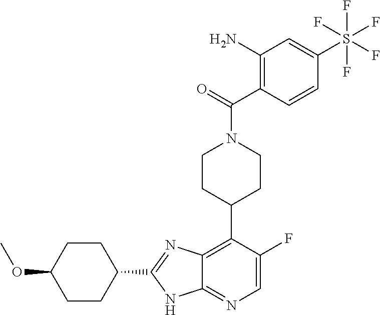 [SMILES: CO[C@H]1CC[C@H](c2nc3c(C4CCN(C(=O)c5ccc(S(F)(F)(F)(F)F)cc5N)CC4)c(F)cnc3[nH]2)CC1] | (trans)-[2-amino-4-(pentafluoro-λ⁶- sulfanyl)phenyl]-[4-[6-fluoro-2-(4- methoxycyclohexyl)-3H- imidazo[4,5-b]pyridin-7-yl]-1- piperidyl]methanone |
| 357 | 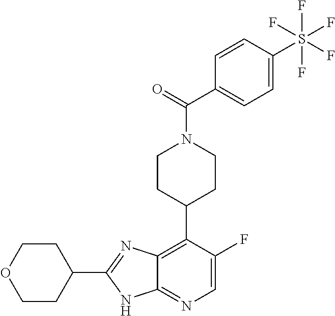 [SMILES: O=C(c1ccc(S(F)(F)(F)(F)F)cc1)N1CCC(c2c(F)cnc3[nH]c(C4CCOCC4)nc23)CC1] | [4-(6-fluoro-2-tetrahydropyran-4-yl- 3H-imidazo[4,5-b]pyridin-7-yl)-1- piperidyl]-[4-(pentafluoro-λ⁶- sulfanyl)phenyl]methanone |
| 358 | 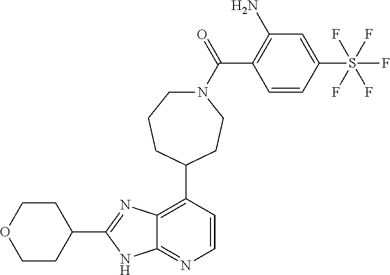 [SMILES: Nc1cc(S(F)(F)(F)(F)F)ccc1C(=O)N1CCCC(c2ccnc3[nH]c(C4CCOCC4)nc23)CC1] Isomer 1 | [2-amino-4-(pentafluoro-λ⁶- sulfanyl)phenyl]-[4-(2- tetrahydropyran-4-yl-3H- imidazo[4,5-b]pyridin-7-yl)azepan-1- yl]methanone, Isomer 1 |
| 359 | 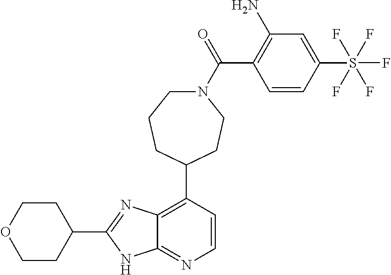 [SMILES: Nc1cc(S(F)(F)(F)(F)F)ccc1C(=O)N1CCCC(c2ccnc3[nH]c(C4CCOCC4)nc23)CC1] Isomer 2 | [2-amino-4-(pentafluoro-λ⁶- sulfanyl)phenyl]-[4-(2- tetrahydropyran-4-yl-3H- imidazo[4,5-b]pyridin-7-yl)azepan-1- yl]methanone, Isomer 2 |
| 360 | 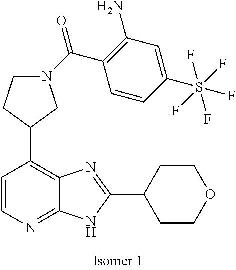 [SMILES: Nc1cc(S(F)(F)(F)(F)F)ccc1C(=O)N1CCC(c2ccnc3[nH]c(C4CCOCC4)nc23)C1] | [2-amino-4-(pentafluoro-λ⁶- sulfanyl)phenyl]-[3-(2- tetrahydropyran-4-yl-3H- imidazo[4,5-b]pyridin-7- yl)pyrrolidin-1-yl]methanone, Isomer 1 |
| 361 | 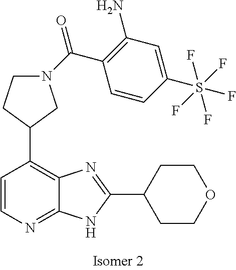 [SMILES: Nc1cc(S(F)(F)(F)(F)F)ccc1C(=O)N1CCC(c2ccnc3[nH]c(C4CCOCC4)nc23)C1] | [2-amino-4-(pentafluoro-λ⁶- sulfanyl)phenyl]-[3-(2- tetrahydropyran-4-yl-3H- imidazo[4,5-b]pyridin-7- yl)pyrrolidin-1-yl]methanone, Isomer 2 |
| 362 | 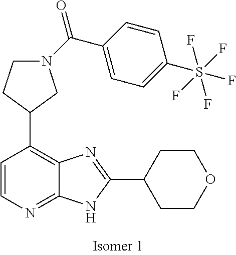 [SMILES: O=C(c1ccc(S(F)(F)(F)(F)F)cc1)N1CCC(c2ccnc3[nH]c(C4CCOCC4)nc23)C1] | [4-(pentafluoro-λ⁶-sulfanyl)phenyl]- [3-(2-tetrahydropyran-4-yl-3H- imidazo[4,5-b]pyridin-7- yl)pyrrolidin-1-yl]methanone, Isomer 1 |
| 363 | 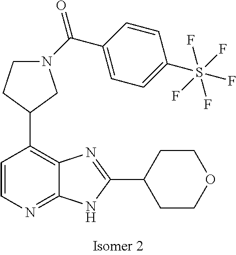 [SMILES: O=C(c1ccc(S(F)(F)(F)(F)F)cc1)N1CCC(c2ccnc3[nH]c(C4CCOCC4)nc23)C1] | [4-(pentafluoro-λ⁶-sulfanyl)phenyl]- [3-(2-tetrahydropyran-4-yl-3H- imidazo[4,5-b]pyridin-7- yl)pyrrolidin-1-yl]methanone, Isomer 2 |
| 364 | 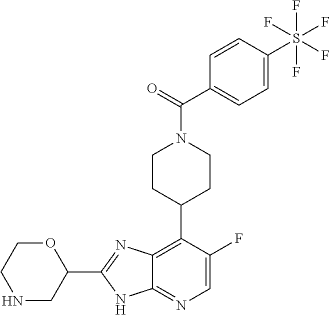 [SMILES: O=C(c1ccc(S(F)(F)(F)(F)F)cc1)N1CCC(c2c(F)cnc3[nH]c(C4CNCCO4)nc23)CC1] | (rac)-[4-(6-fluoro-2-morpholin-2-yl- 3H-imidazo[4,5-b]pyridin-7-yl)-1- piperidyl]-[4-(pentafluoro-λ⁶- sulfanyl)phenyl]methanone |
| 365 | 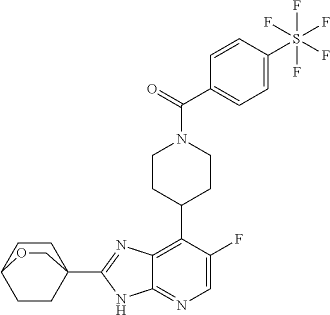 [SMILES: O=C(c1ccc(S(F)(F)(F)(F)F)cc1)N1CCC(c2c(F)cnc3[nH]c(C45CCC(CC4)OC5)nc23)CC1] | [4-[6-fluoro-2-(2- oxabicyclo[2.2.2]octan-4-yl)-3H- imidazo[4,5-b]pyridin-7-yl]-1- piperidyl]-[4-(pentafluoro-λ⁶- sulfanyl)phenyl]methanone |
| 366 | 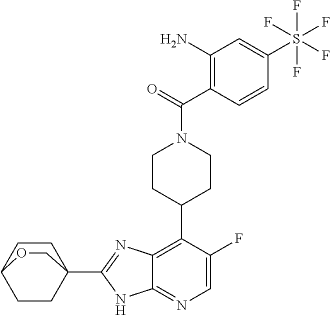 [SMILES: Nc1cc(S(F)(F)(F)(F)F)ccc1C(=O)N1CCC(c2c(F)cnc3[nH]c(C45CCC(CC4)OC5)nc23)CC1] | [2-amino-4-(pentafluoro-λ⁶- sulfanyl)phenyl]-[4-[6-fluoro-2-(2- oxabicyclo[2.2.2]octan-4-yl)-3H- imidazo[4,5-b]pyridin-7-yl]-1- piperidyl]methanone |
| 367 | 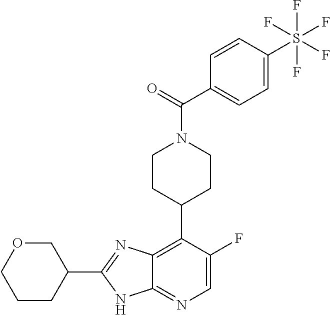 [SMILES: O=C(c1ccc(S(F)(F)(F)(F)F)cc1)N1CCC(c2c(F)cnc3[nH]c(C4CCCOC4)nc23)CC1] | (rac)-[4-(6-fluoro-2-tetrahydropyran- 3-yl-3H-imidazo[4,5-b]pyridin-7-yl)- 1-piperidyl]-[4-(pentafluoro-λ⁶- sulfanyl)phenyl]methanone |
| 368 | 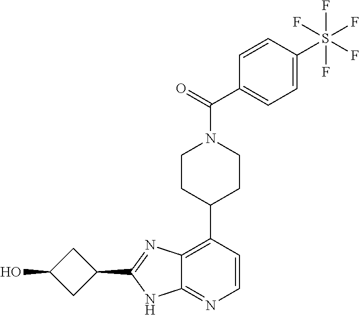 [SMILES: O=C(c1ccc(S(F)(F)(F)(F)F)cc1)N1CCC(c2ccnc3[nH]c([C@H]4C[C@@H](O)C4)nc23)CC1] | (cis)-[4-[2-(3-hydroxycyclobutyl)- 3H-imidazo[4,5-b]pyridin-7-yl]-1- piperidyl]-[4-(pentafluoro-λ⁶- sulfanyl)phenyl]methanone |
| 369 | 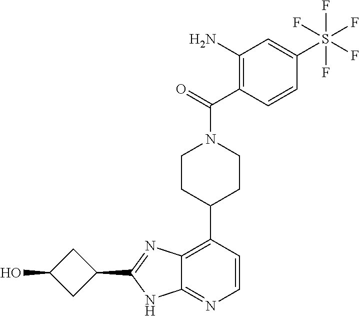 [SMILES: Nc1cc(S(F)(F)(F)(F)F)ccc1C(=O)N1CCC(c2ccnc3[nH]c([C@H]4C[C@@H](O)C4)nc23)CC1] | (cis)-[2-amino-4-(pentafluoro-λ⁶- sulfanyl)phenyl]-[4-[2-(3- hydroxycyclobutyl)-3H-imidazo[4,5- b]pyridin-7-yl]-1- piperidyl]methanone |
| 370 | 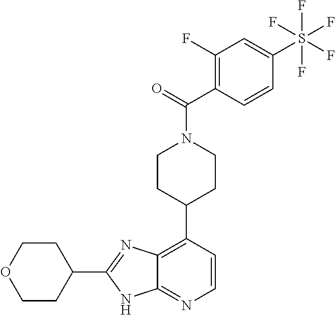 [SMILES: O=C(c1ccc(S(F)(F)(F)(F)F)cc1F)N1CCC(c2ccnc3[nH]c(C4CCOCC4)nc23)CC1] | [2-fluoro-4-(pentafluoro-λ⁶- sulfanyl)phenyl]-[4-(2- tetrahydropyran-4-yl-3H- imidazo[4,5-b]pyridin-7-yl)-1- piperidyl]methanone |
| 371 | 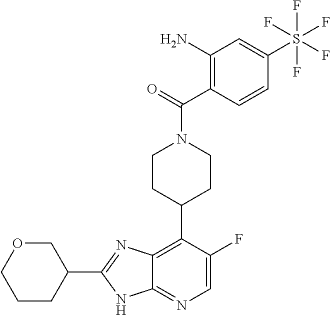 [SMILES: Nc1cc(S(F)(F)(F)(F)F)ccc1C(=O)N1CCC(c2c(F)cnc3[nH]c(C4CCCOC4)nc23)CC1] | (rac)-[2-amino-4-(pentafluoro-λ⁶- sulfanyl)phenyl]-[4-(6-fluoro-2- tetrahydropyran-3-yl-3H- imidazo[4,5-b]pyridin-7-yl)-1- piperidyl]methanone |
| 372 | 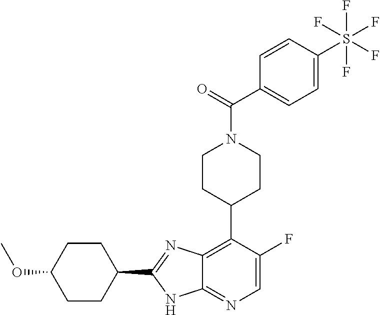 [SMILES: CO[C@H]1CC[C@H](c2nc3c(C4CCN(C(=O)c5ccc(S(F)(F)(F)(F)F)cc5)CC4)c(F)cnc3[nH]2)CC1] | (trans)-[4-[6-fluoro-2-(4- methoxycyclohexyl)-3H- imidazo[4,5-b]pyridin-7-yl]-1- piperidyl]-[4-(pentafluoro-λ⁶- sulfanyl)phenyl]methanone |
| 373 | 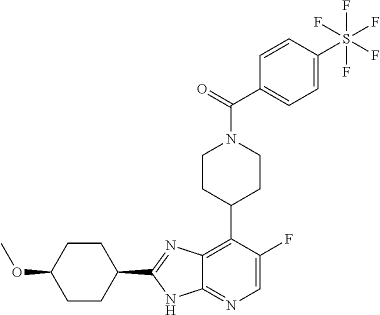 [SMILES: CO[C@H]1CC[C@@H](c2nc3c(C4CCN(C(=O)c5ccc(S(F)(F)(F)(F)F)cc5)CC4)c(F)cnc3[nH]2)CC1] | (cis)-[4-[6-fluoro-2-(4- methoxycyclohexyl)-3H- imidazo[4,5-b]pyridin-7-yl]-1- piperidyl]-[4-(pentafluoro-λ⁶- sulfanyl)phenyl]methanone |
| 374 | 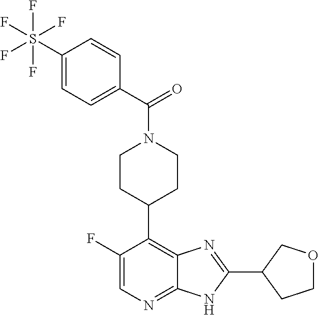 [SMILES: O=C(c1ccc(S(F)(F)(F)(F)F)cc1)N1CCC(c2c(F)cnc3[nH]c(C4CCOC4)nc23)CC1] | (rac)-[4-(6-fluoro-2-tetrahydrofuran- 3-yl-3H-imidazo[4,5-b]pyridin-7-yl)- 1-piperidyl]-[4-(pentafluoro-λ⁶- sulfanyl)phenyl]methanone |
| 375 | 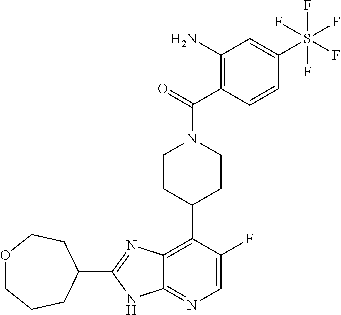 [SMILES: Nc1cc(S(F)(F)(F)(F)F)ccc1C(=O)N1CCC(c2c(F)cnc3[nH]c(C4CCCOCC4)nc23)CC1] | (rac)-[2-amino-4-(pentafluoro-λ⁶- sulfanyl)phenyl]-[4-[6-fluoro-2- (oxepan-4-yl)-3H-imidazo[4,5- b]pyridin-7-yl]-1- piperidyl]methanone |
| 376 | 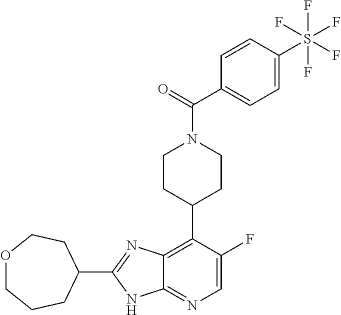 [SMILES: O=C(c1ccc(S(F)(F)(F)(F)F)cc1)N1CCC(c2c(F)cnc3[nH]c(C4CCCOCC4)nc23)CC1] | (rac)-[4-[6-fluoro-2-(oxepan-4-yl)- 3H-imidazo[4,5-b]pyridin-7-yl]-1- piperidyl]-[4-(pentafluoro-λ⁶- sulfanyl)phenyl]methanone |
| 377 | 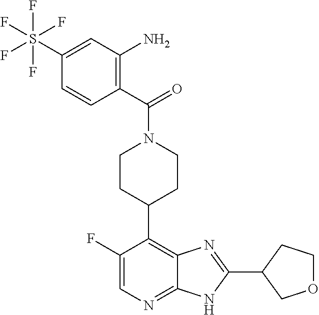 [SMILES: Nc1cc(S(F)(F)(F)(F)F)ccc1C(=O)N1CCC(c2c(F)cnc3[nH]c(C4CCOC4)nc23)CC1] | (rac)-[2-amino-4-(pentafluoro-λ⁶- sulfanyl)phenyl]-[4-(6-fluoro-2- tetrahydrofuran-3-yl-3H- imidazo[4,5-b]pyridin-7-yl)-1- piperidyl]methanone |
| 378 | 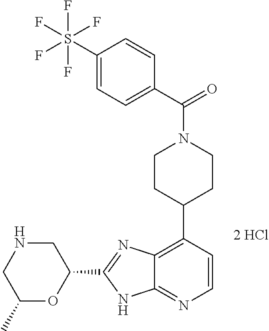 [SMILES: C[C@@H]1CNC[C@H](c2nc3c(C4CCN(C(=O)c5ccc(S(F)(F)(F)(F)F)cc5)CC4)ccnc3[nH]2)O1] | [4-[2-[(2R,6R)-6-methylmorpholin- 2-yl]-3H-imidazo[4,5-b]pyridin-7- yl]-1-piperidyl]-[4-(pentafluoro-λ⁶- sulfanyl)phenyl]methanone; dihydrochloride |
| 379 | 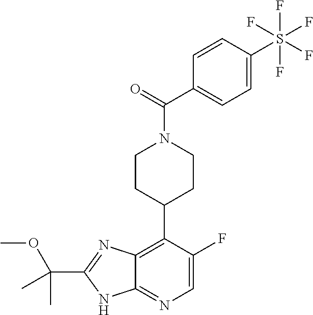 [SMILES: COC(C)(C)c1nc2c(C3CCN(C(=O)c4ccc(S(F)(F)(F)(F)F)cc4)CC3)c(F)cnc2[nH]1] | [4-[6-fluoro-2-(1-methoxy-1-methyl- ethyl)-3H-imidazo[4,5-b]pyridin-7- yl]-1-piperidyl]-[4-(pentafluoro-λ⁶- sulfanyl)phenyl]methanone |
| 380 | 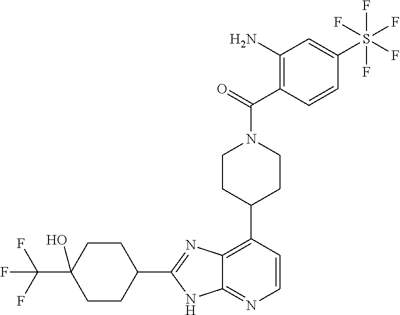 [SMILES: Nc1cc(S(F)(F)(F)(F)F)ccc1C(=O)N1CCC(c2ccnc3[nH]c(C4CCC(O)(C(F)(F)F)CC4)nc23)CC1] | [2-amino-4-(pentafluoro-λ⁶- sulfanyl)phenyl]-[4-[2-[4-hydroxy-4- (trifluoromethyl)cyclohexyl]-3H- imidazo[4,5-b]pyridin-7-yl]-1- piperidyl]methanone |
| 381 | 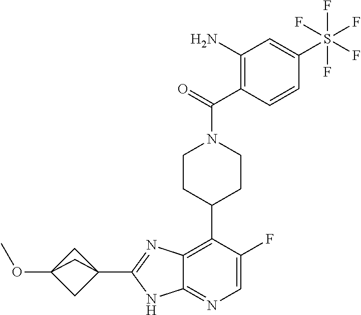 [SMILES: COC12CC(c3nc4c(C5CCN(C(=O)c6ccc(S(F)(F)(F)(F)F)cc6N)CC5)c(F)cnc4[nH]3)(C1)C2] | [2-amino-4-(pentafluoro-λ⁶- sulfanyl)phenyl]-[4-[6-fluoro-2-(3- methoxy-1-bicyclo[1.1.1]pentanyl)- 3H-imidazo[4,5-b]pyridin-7-yl]-1- piperidyl]methanone |
| 382 | 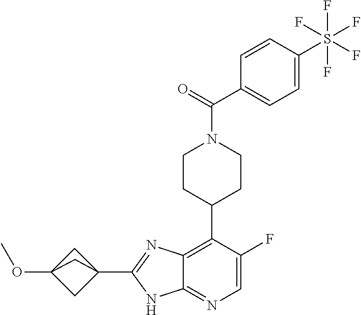 [SMILES: COC12CC(c3nc4c(C5CCN(C(=O)c6ccc(S(F)(F)(F)(F)F)cc6)CC5)c(F)cnc4[nH]3)(C1)C2] | [4-[6-fluoro-2-(3-methoxy-1- bicyclo[1.1.1]pentanyl)-3H- imidazo[4,5-b]pyridin-7-yl]-1- piperidyl]-[4-(pentafluoro-λ⁶- sulfanyl)phenyl]methanone |
| 383 | 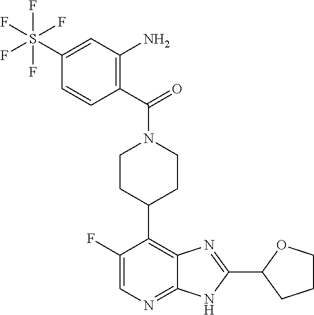 [SMILES: Nc1cc(S(F)(F)(F)(F)F)ccc1C(=O)N1CCC(c2c(F)cnc3[nH]c(C4CCCO4)nc23)CC1] | (rac)-[2-amino-4-(pentafluoro-λ⁶- sulfanyl)phenyl]-[4-(6-fluoro-2- tetrahydrofuran-2-yl-3H- imidazo[4,5-b]pyridin-7-yl)-1- piperidyl]methanone |
| 384 | 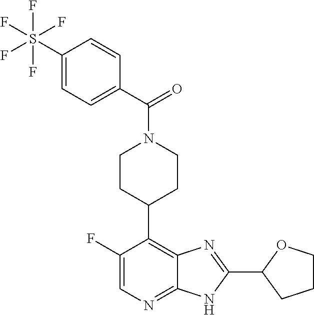 [SMILES: O=C(c1ccc(S(F)(F)(F)(F)F)cc1)N1CCC(c2c(F)cnc3[nH]c(C4CCCO4)nc23)CC1] | (rac)-[4-(6-fluoro-2-tetrahydrofuran- 2-yl-3H-imidazo[4,5-b]pyridin-7-yl)- 1-piperidyl]-[4-(pentafluoro-λ⁶- sulfanyl)phenyl]methanone |
| 385 | 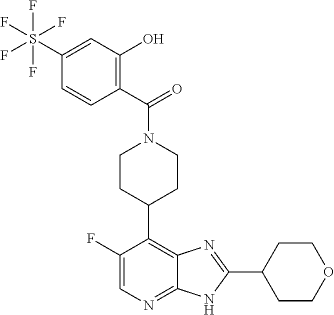 [SMILES: O=C(c1ccc(S(F)(F)(F)(F)F)cc1O)N1CCC(c2c(F)cnc3[nH]c(C4CCOCC4)nc23)CC1] | [4-(6-fluoro-2-tetrahydropyran-4-yl- 3H-imidazo[4,5-b]pyridin-7-yl)-1- piperidyl]-[2-hydroxy-4- (pentafluoro-λ⁶- sulfanyl)phenyl]methanone |
| 386 | 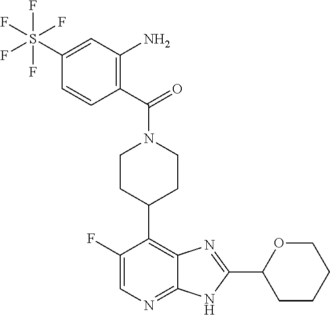 [SMILES: Nc1cc(S(F)(F)(F)(F)F)ccc1C(=O)N1CCC(c2c(F)cnc3[nH]c(C4CCCCO4)nc23)CC1] | (rac)-[2-amino-4-(pentafluoro-λ⁶- sulfanyl)phenyl]-[4-(6-fluoro-2- tetrahydropyran-2-yl-3H- imidazo[4,5-b]pyridin-7-yl)-1- piperidyl]methanone |
| 387 | 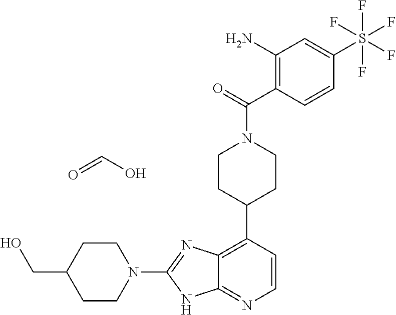 [SMILES: Nc1cc(S(F)(F)(F)(F)F)ccc1C(=O)N1CCC(c2ccnc3[nH]c(N4CCC(CO)CC4)nc23)CC1 ; O=CO] | [2-amino-4-(pentafluoro-λ⁶- sulfanyl)phenyl]-[4-[2-[4- (hydroxymethyl)-1-piperidyl]-3H- imidazo[4,5-b]pyridin-7-yl]-1- piperidyl]methanone;formic acid |
| 388 | 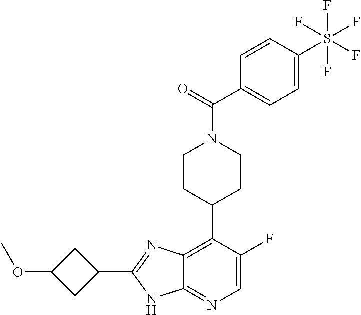 [SMILES: COC1CC(c2nc3c(C4CCN(C(=O)c5ccc(S(F)(F)(F)(F)F)cc5)CC4)c(F)cnc3[nH]2)C1] | [4-[6-fluoro-2-(3- methoxycyclobutyl)-3H-imidazo[4,5- b]pyridin-7-yl]-1-piperidyl]-[4- (pentafluoro-λ⁶- sulfanyl)phenyl]methanone |
| 389 | 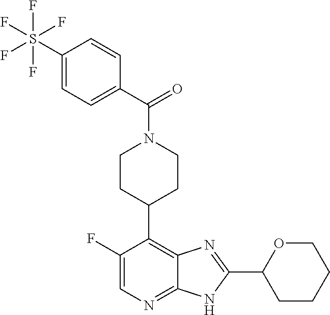 [SMILES: O=C(c1ccc(S(F)(F)(F)(F)F)cc1)N1CCC(c2c(F)cnc3[nH]c(C4CCCCO4)nc23)CC1] | (rac)-[4-(6-fluoro-2-tetrahydropyran- 2-yl-3H-imidazo[4,5-b]pyridin-7-yl)- 1-piperidyl]-[4-(pentafluoro-λ⁶- sulfanyl)phenyl]methanone |
| 390 | 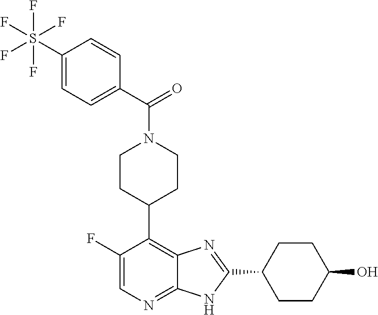 [SMILES: O=C(c1ccc(S(F)(F)(F)(F)F)cc1)N1CCC(c2c(F)cnc3[nH]c([C@H]4CC[C@H](O)CC4)nc23)CC1] | (trans)-[4-[6-fluoro-2-(4- hydroxycyclohexyl)-3H-imidazo[4,5- b]pyridin-7-yl]-1-piperidyl]-[4- (pentafluoro-λ⁶- sulfanyl)phenyl]methanone |
| 391 | 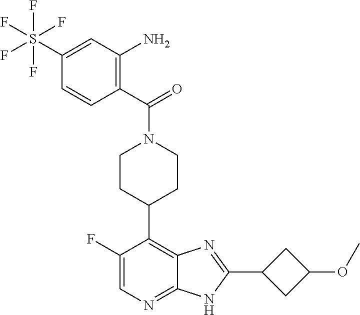 [SMILES: COC1CC(c2nc3c(C4CCN(C(=O)c5ccc(S(F)(F)(F)(F)F)cc5N)CC4)c(F)cnc3[nH]2)C1] | [2-amino-4-(pentafluoro-λ⁶- sulfanyl)phenyl]-[4-[6-fluoro-2-(3- methoxycyclobutyl)-3H-imidazo[4,5- b]pyridin-7-yl]-1- piperidyl]methanone |
| 392 | 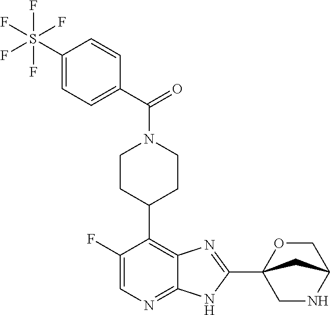 [SMILES: O=C(c1ccc(S(F)(F)(F)(F)F)cc1)N1CCC(c2c(F)cnc3[nH]c([C@@]45CN[C@@H](CO4)C5)nc23)CC1] | [4-[6-fluoro-2-[(1R,4R)-2-oxa-5- azabicyclo[2.2.1]heptan-1-yl]-3H- imidazo[4,5-b]pyridin-7-yl]-1- piperidyl]-[4-(pentafluoro-λ⁶- sulfanyl)phenyl]methanone |
| 393 | 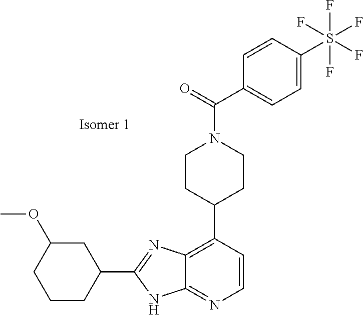 [SMILES: COC1CCCC(c2nc3c(C4CCN(C(=O)c5ccc(S(F)(F)(F)(F)F)cc5)CC4)ccnc3[nH]2)C1] | [4-[2-[3-methoxycyclohexyl]-3H- imidazo[4,5-b]pyridin-7-yl]-1- piperidyl]-[4-(pentafluoro-λ⁶- sulfanyl)phenyl]methanone, Isomer 1 |
| 394 | 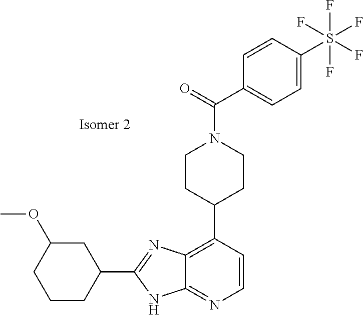 [SMILES: COC1CCCC(c2nc3c(C4CCN(C(=O)c5ccc(S(F)(F)(F)(F)F)cc5)CC4)ccnc3[nH]2)C1] | [4-[2-[3-methoxycyclohexyl]-3H- imidazo[4,5-b]pyridin-7-yl]-1- piperidyl]-[4-(pentafluoro-λ⁶- sulfanyl)phenyl]methanone, Isomer 2 |
| 395 | 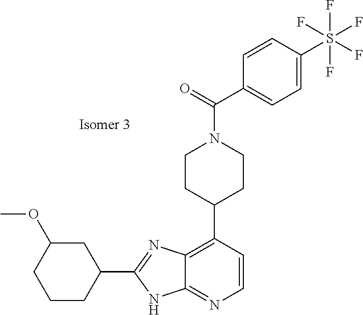 [SMILES: COC1CCCC(c2nc3c(C4CCN(C(=O)c5ccc(S(F)(F)(F)(F)F)cc5)CC4)ccnc3[nH]2)C1] | [4-[2-[3-methoxycyclohexyl]-3H- imidazo[4,5-b]pyridin-7-yl]-1- piperidyl]-[4-(pentafluoro-λ⁶- sulfanyl)phenyl]methanone, Isomer 3 |
| 396 | 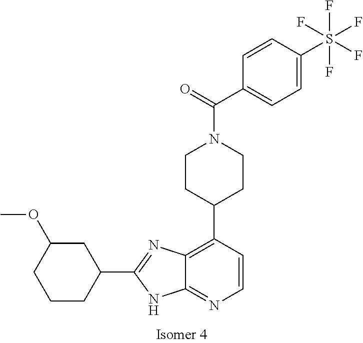 [SMILES: COC1CCCC(c2nc3c(C4CCN(C(=O)c5ccc(S(F)(F)(F)(F)F)cc5)CC4)ccnc3[nH]2)C1] | [4-[2-[(3-methoxycyclohexyl]-3H- imidazo[4,5-b]pyridin-7-yl]-1- piperidyl]-[4-(pentafluoro-λ⁶- sulfanyl)phenyl]methanone, Isomer 4 |
| 397 | 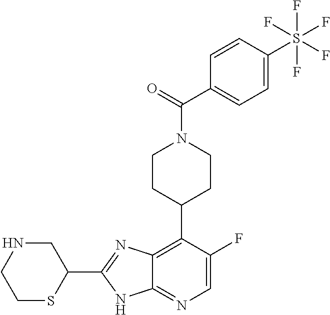 [SMILES: O=C(c1ccc(S(F)(F)(F)(F)F)cc1)N1CCC(c2c(F)cnc3[nH]c(C4CNCCS4)nc23)CC1] | (rac)-[4-(6-fluoro-2-thiomorpholin-2- yl-3H-imidazo[4,5-b]pyridin-7-yl)-1- piperidyl]-[4-(pentafluoro-λ⁶- sulfanyl)phenyl]methanone |
| 398 | 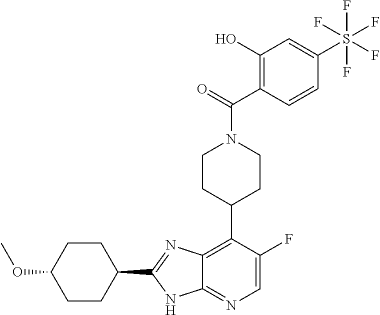 [SMILES: CO[C@H]1CC[C@H](c2nc3c(C4CCN(C(=O)c5ccc(S(F)(F)(F)(F)F)cc5O)CC4)c(F)cnc3[nH]2)CC1] | (trans)-[4-[6-fluoro-2-(4- methoxycyclohexyl)-3H- imidazo[4,5-b]pyridin-7-yl]-1- piperidyl]-[2-hydroxy-4- (pentafluoro-λ⁶- sulfanyl)phenyl]methanone |
| 399 | 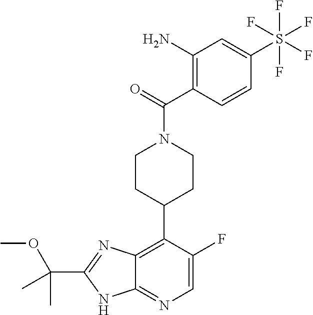 [SMILES: COC(C)(C)c1nc2c(C3CCN(C(=O)c4ccc(S(F)(F)(F)(F)F)cc4N)CC3)c(F)cnc2[nH]1] | [2-amino-4-(pentafluoro-λ⁶- sulfanyl)phenyl]-[4-[6-fluoro-2-(1- methoxy-1-methyl-ethyl)-3H- imidazo[4,5-b]pyridin-7-yl]-1- piperidyl]methanone |
| 400 | 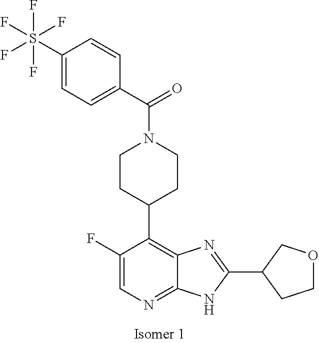 [SMILES: O=C(c1ccc(S(F)(F)(F)(F)F)cc1)N1CCC(c2c(F)cnc3[nH]c(C4CCOC4)nc23)CC1] | [4-[6-fluoro-2-[tetrahydrofuran-3-yl]- 3H-imidazo[4,5-b]pyridin-7-yl]-1- piperidyl]-[4-(pentafluoro-λ⁶- sulfanyl)phenyl]methanone, Isomer 1 |
| 401 | 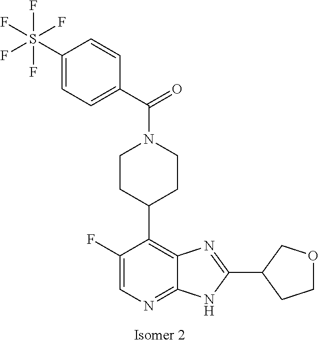 [SMILES: O=C(c1ccc(S(F)(F)(F)(F)F)cc1)N1CCC(c2c(F)cnc3[nH]c(C4CCOC4)nc23)CC1] | [4-[6-fluoro-2-[tetrahydrofuran-3-yl]- 3H-imidazo[4,5-b]pyridin-7-yl]-1- piperidyl]-[4-(pentafluoro-λ⁶- sulfanyl)phenyl]methanone, Isomer 2 |
| 402 | 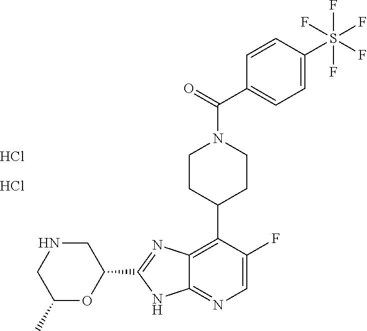 [SMILES: Cl ; Cl ; C[C@@H]1CNC[C@H](c2nc3c(C4CCN(C(=O)c5ccc(S(F)(F)(F)(F)F)cc5)CC4)c(F)cnc3[nH]2)O1] | [4-[6-fluoro-2-[(2R,6R)-6- methylmorpholin-2-yl]-3H- imidazo[4,5-b]pyridin-7-yl]-1- piperidyl]-[4-(pentafluoro-λ⁶- sulfanyl)phenyl]methanone; dihydrochloride |
| 403 | 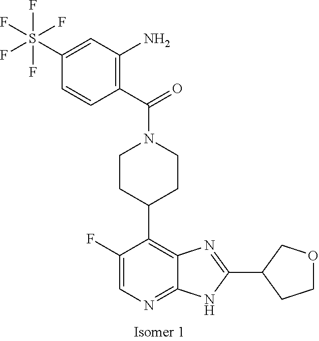 [SMILES: Nc1cc(S(F)(F)(F)(F)F)ccc1C(=O)N1CCC(c2c(F)cnc3[nH]c(C4CCOC4)nc23)CC1] | [2-amino-4-(pentafluoro-λ⁶- sulfanyl)phenyl]-[4-[6-fluoro-2- [tetrahydrofuran-3-yl]-3H- imidazo[4,5-b]pyridin-7-yl]-1- piperidyl]methanone, Isomer 1 |
| 404 | 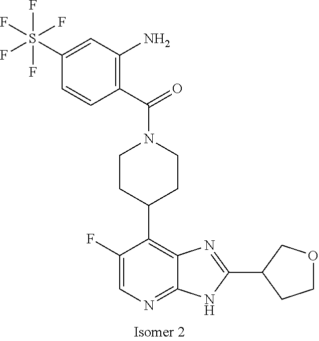 [SMILES: Nc1cc(S(F)(F)(F)(F)F)ccc1C(=O)N1CCC(c2c(F)cnc3[nH]c(C4CCOC4)nc23)CC1] | [2-amino-4-(pentafluoro-λ⁶- sulfanyl)phenyl]-[4-[6-fluoro-2- [tetrahydrofuran-3-yl]-3H- imidazo[4,5-b]pyridin-7-yl]-1- piperidyl]methanone, Isomer 2 |
| 405 | 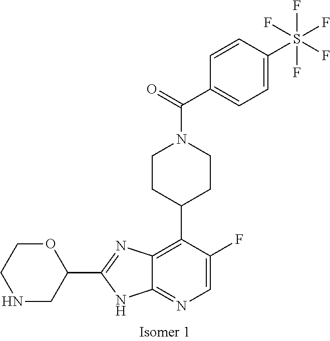 [SMILES: O=C(c1ccc(S(F)(F)(F)(F)F)cc1)N1CCC(c2c(F)cnc3[nH]c(C4CNCCO4)nc23)CC1] | [4-[6-fluoro-2-[morpholin-2-yl]-3H- imidazo[4,5-b]pyridin-7-yl]-1- piperidyl]-[4-(pentafluoro-λ⁶- sulfanyl)phenyl]methanone, Isomer 1 |
| 406 | 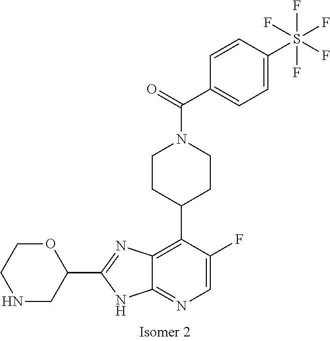 [SMILES: O=C(c1ccc(S(F)(F)(F)(F)F)cc1)N1CCC(c2c(F)cnc3[nH]c(C4CNCCO4)nc23)CC1] | [4-[6-fluoro-2-[morpholin-2-yl]-3H- imidazo[4,5-b]pyridin-7-yl]-1- piperidyl]-[4-(pentafluoro-λ⁶- sulfanyl)phenyl]methanone, Isomer 2 |
| 407 | 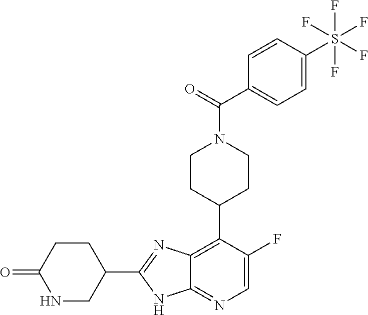 [SMILES: O=C1CCC(c2nc3c(C4CCN(C(=O)c5ccc(S(F)(F)(F)(F)F)cc5)CC4)c(F)cnc3[nH]2)CN1] | (rac)-5-[6-fluoro-7-[1-[4- (pentafluoro-λ⁶-sulfanyl)benzoyl]-4- piperidyl]-3H-imidazo[4,5-b]pyridin- 2-yl]piperidin-2-one |
| 408 | 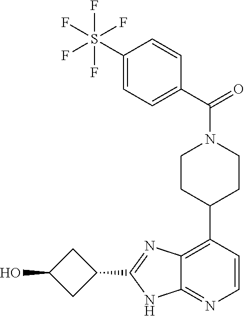 [SMILES: O=C(c1ccc(S(F)(F)(F)(F)F)cc1)N1CCC(c2ccnc3[nH]c([C@H]4C[C@H](O)C4)nc23)CC1] | (trans)-[4-[2-(3-hydroxycyclobutyl)- 3H-imidazo[4,5-b]pyridin-7-yl]-1- piperidyl]-[4-(pentafluoro-λ⁶- sulfanyl)phenyl]methanone |
| 409 | 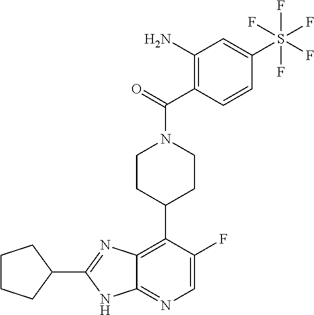 [SMILES: Nc1cc(S(F)(F)(F)(F)F)ccc1C(=O)N1CCC(c2c(F)cnc3[nH]c(C4CCCC4)nc23)CC1] | [2-amino-4-(pentafluoro-λ⁶- sulfanyl)phenyl]-[4-(2-cyclopentyl-6- fluoro-3H-imidazo[4,5-b]pyridin-7- yl)-1-piperidyl]methanone |
| 410 | 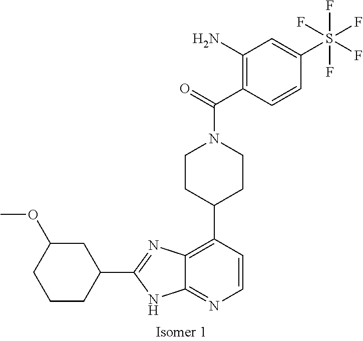 [SMILES: COC1CCCC(c2nc3c(C4CCN(C(=O)c5ccc(S(F)(F)(F)(F)F)cc5N)CC4)ccnc3[nH]2)C1] | [2-amino-4-(pentafluoro-λ⁶- sulfanyl)phenyl]-[4-[2-[3- methoxycyclohexyl]-3H- imidazo[4,5-b]pyridin-7-yl]-1- piperidyl]methanone, Isomer 1 |
| 411 | 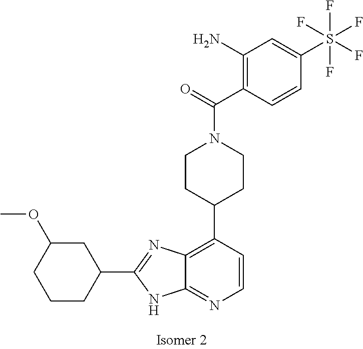 [SMILES: COC1CCCC(c2nc3c(C4CCN(C(=O)c5ccc(S(F)(F)(F)(F)F)cc5N)CC4)ccnc3[nH]2)C1] | [2-amino-4-(pentafluoro-λ⁶- sulfanyl)phenyl]-[4-[2-[3- methoxycyclohexyl]-3H- imidazo[4,5-b]pyridin-7-yl]-1- piperidyl]methanone, Isomer 2 |
| 412 | 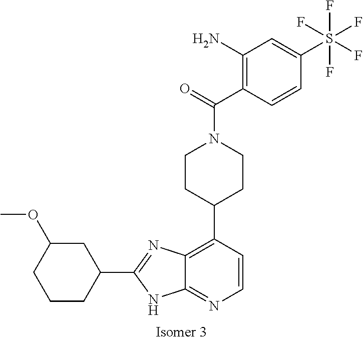 [SMILES: COC1CCCC(c2nc3c(C4CCN(C(=O)c5ccc(S(F)(F)(F)(F)F)cc5N)CC4)ccnc3[nH]2)C1] | [2-amino-4-(pentafluoro-λ⁶- sulfanyl)phenyl]-[4-[2-[3- methoxycyclohexyl]-3H- imidazo[4,5-b]pyridin-7-yl]-1- piperidyl]methanone, Isomer 3 |
| 413 | 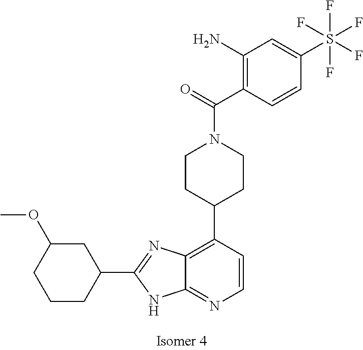 [SMILES: COC1CCCC(c2nc3c(C4CCN(C(=O)c5ccc(S(F)(F)(F)(F)F)cc5N)CC4)ccnc3[nH]2)C1] | [2-amino-4-(pentafluoro-λ⁶- sulfanyl)phenyl]-[4-[2-[3- methoxycyclohexyl]-3H- imidazo[4,5-b]pyridin-7-yl]-1- piperidyl]methanone, Isomer 4 |
| 414 | 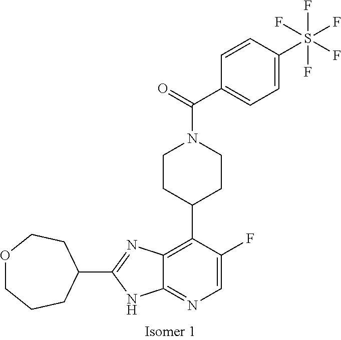 [SMILES: O=C(c1ccc(S(F)(F)(F)(F)F)cc1)N1CCC(c2c(F)cnc3[nH]c(C4CCCOCC4)nc23)CC1] | [4-[6-fluoro-2-[oxepan-4-yl]-3H- imidazo[4,5-b]pyridin-7-yl]-1- piperidyl]-[4-(pentafluoro-λ⁶- sulfanyl)phenyl]methanone, Isomer 1 |
| 415 | 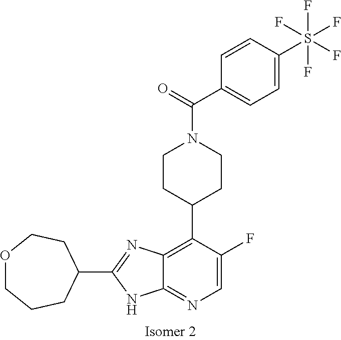 [SMILES: O=C(c1ccc(S(F)(F)(F)(F)F)cc1)N1CCC(c2c(F)cnc3[nH]c(C4CCCOCC4)nc23)CC1] | [4-[6-fluoro-2-[oxepan-4-yl]-3H- imidazo[4,5-b]pyridin-7-yl]-1- piperidyl]-[4-(pentafluoro-λ⁶- sulfanyl)phenyl]methanone, Isomer 2 |
| 416 | 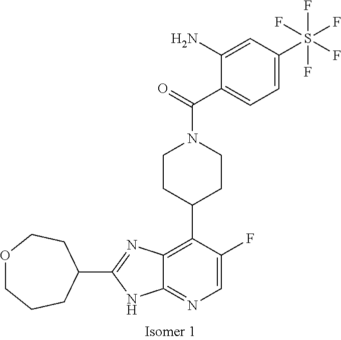 [SMILES: Nc1cc(S(F)(F)(F)(F)F)ccc1C(=O)N1CCC(c2c(F)cnc3[nH]c(C4CCCOCC4)nc23)CC1] | [2-amino-4-(pentafluoro-λ⁶- sulfanyl)phenyl]-[4-[6-fluoro-2- [oxepan-4-yl]-3H-imidazo[4,5- b]pyridin-7-yl]-1- piperidyl]methanone, Isomer 1 |
| 417 | 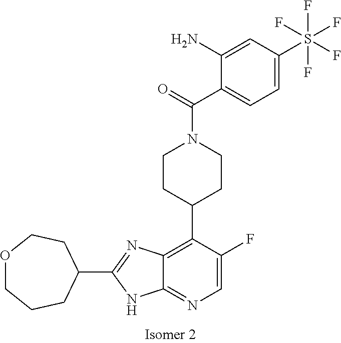 [SMILES: Nc1cc(S(F)(F)(F)(F)F)ccc1C(=O)N1CCC(c2c(F)cnc3[nH]c(C4CCCOCC4)nc23)CC1] | [2-amino-4-(pentafluoro-λ⁶- sulfanyl)phenyl]-[4-[6-fluoro-2- [oxepan-4-yl]-3H-imidazo[4,5- b]pyridin-7-yl]-1- piperidyl]methanone, Isomer 2 |
| 418 | 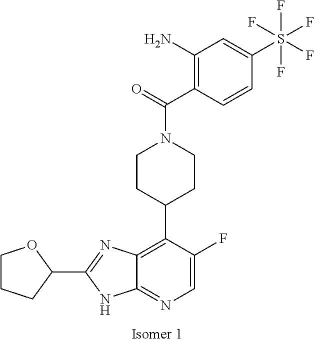 [SMILES: Nc1cc(S(F)(F)(F)(F)F)ccc1C(=O)N1CCC(c2c(F)cnc3[nH]c(C4CCCO4)nc23)CC1] | [2-amino-4-(pentafluoro-λ⁶- sulfanyl)phenyl]-[4-[6-fluoro-2- [tetrahydrofuran-2-yl]-3H- imidazo[4,5-b]pyridin-7-yl]-1- piperidyl]methanone, Isomer 1 |
| 419 | 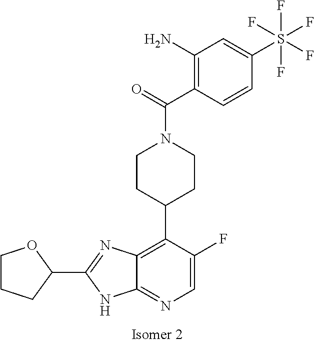 [SMILES: Nc1cc(S(F)(F)(F)(F)F)ccc1C(=O)N1CCC(c2c(F)cnc3[nH]c(C4CCCO4)nc23)CC1] | [2-amino-4-(pentafluoro-λ⁶- sulfanyl)phenyl]-[4-[6-fluoro-2- [tetrahydrofuran-2-yl]-3H- imidazo[4,5-b]pyridin-7-yl]-1- piperidyl]methanone, Isomer 2 |
| 420 | 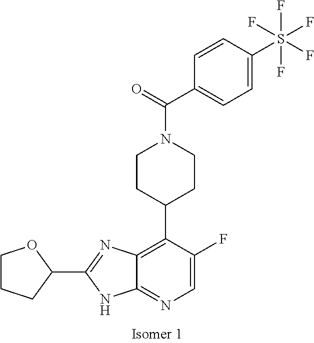 [SMILES: O=C(c1ccc(S(F)(F)(F)(F)F)cc1)N1CCC(c2c(F)cnc3[nH]c(C4CCCO4)nc23)CC1] | [4-[6-fluoro-2-[tetrahydrofuran-2-yl]- 3H-imidazo[4,5-b]pyridin-7-yl]-1- piperidyl]-[4-(pentafluoro-λ⁶- sulfanyl)phenyl]methanone, Isomer 1 |
| 421 | 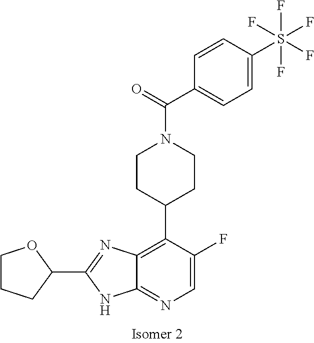 [SMILES: O=C(c1ccc(S(F)(F)(F)(F)F)cc1)N1CCC(c2c(F)cnc3[nH]c(C4CCCO4)nc23)CC1] | [4-[6-fluoro-2-[tetrahydrofuran-2-yl]- 3H-imidazo[4,5-b]pyridin-7-yl]-1- piperidyl]-[4-(pentafluoro-λ⁶- sulfanyl)phenyl]methanone, Isomer 2 |
| 422 | 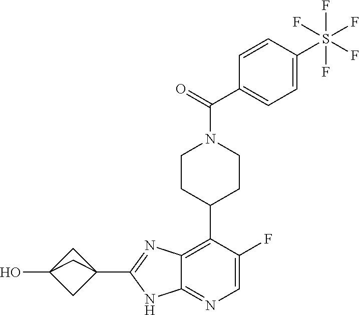 [SMILES: O=C(c1ccc(S(F)(F)(F)(F)F)cc1)N1CCC(c2c(F)cnc3[nH]c(C45CC(O)(C4)C5)nc23)CC1] | [4-[6-fluoro-2-(3-hydroxy-1- bicyclo[1.1.1]pentanyl)-3H- imidazo[4,5-b]pyridin-7-yl]-1- piperidyl]-[4-(pentafluoro-λ⁶- sulfanyl)phenyl]methanone |
| 423 | 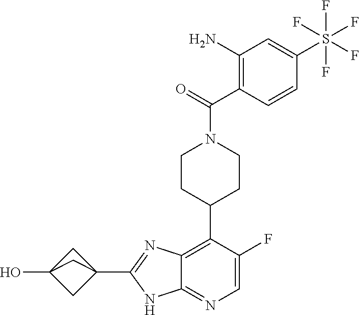 [SMILES: Nc1cc(S(F)(F)(F)(F)F)ccc1C(=O)N1CCC(c2c(F)cnc3[nH]c(C45CC(O)(C4)C5)nc23)CC1] | [2-amino-4-(pentafluoro-λ⁶- sulfanyl)phenyl]-[4-[6-fluoro-2-(3- hydroxy-1-bicyclo[1.1.1]pentanyl)- 3H-imidazo[4,5-b]pyridin-7-yl]-1- piperidyl]methanone |
| 424 | 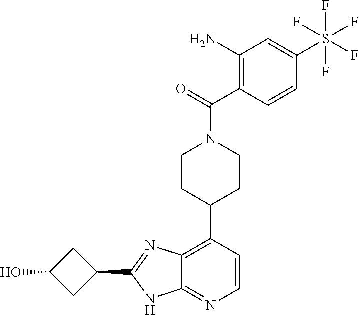 [SMILES: Nc1cc(S(F)(F)(F)(F)F)ccc1C(=O)N1CCC(c2ccnc3[nH]c([C@H]4C[C@H](O)C4)nc23)CC1] | (trans)-[2-amino-4-(pentafluoro-λ⁶- sulfanyl)phenyl]-[4-[2-(3- hydroxycyclobutyl)-3H-imidazo[4,5- b]pyridin-7-yl]-1- piperidyl]methanone |
| 425 | 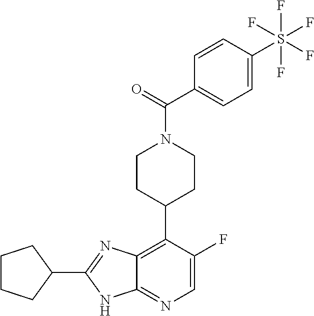 [SMILES: O=C(c1ccc(S(F)(F)(F)(F)F)cc1)N1CCC(c2c(F)cnc3[nH]c(C4CCCC4)nc23)CC1] | [4-(2-cyclopentyl-6-fluoro-3H- imidazo[4,5-b]pyridin-7-yl)-1- piperidyl]-[4-(pentafluoro-λ⁶- sulfanyl)phenyl]methanone |
| 426 | 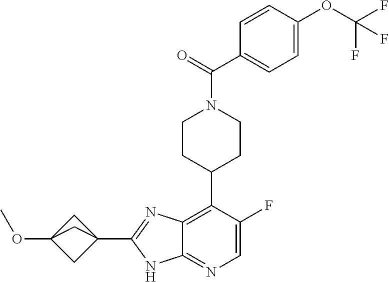 [SMILES: COC12CC(c3nc4c(C5CCN(C(=O)c6ccc(OC(F)(F)F)cc6)CC5)c(F)cnc4[nH]3)(C1)C2] | [4-[6-fluoro-2-(3-methoxy-1- bicyclo[1.1.1]pentanyl)-3H- imidazo[4,5-b]pyridin-7-yl]-1- piperidyl]-[4- (trifluoromethoxy)phenyl]methanone |
| 427 | 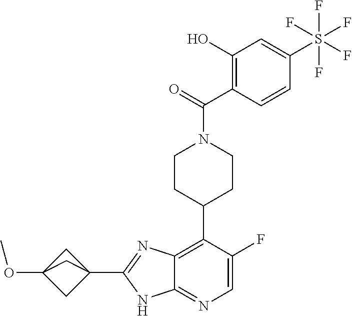 [SMILES: COC12CC(c3nc4c(C5CCN(C(=O)c6ccc(S(F)(F)(F)(F)F)cc6O)CC5)c(F)cnc4[nH]3)(C1)C2] | [4-[6-fluoro-2-(3-methoxy-1- bicyclo[1.1.1]pentanyl)-3H- imidazo[4,5-b]pyridin-7-yl]-1- piperidyl]-[2-hydroxy-4- (pentafluoro-λ⁶- sulfanyl)phenyl]methanone |
| 428 | 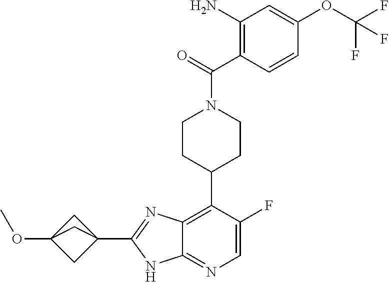 [SMILES: COC12CC(c3nc4c(C5CCN(C(=O)c6ccc(OC(F)(F)F)cc6N)CC5)c(F)cnc4[nH]3)(C1)C2] | [2-amino-4- (trifluoromethoxy)phenyl]-[4-[6- fluoro-2-(3-methoxy-1- bicyclo[1.1.1]pentanyl)-3H- imidazo[4,5-b]pyridin-7-yl]-1- piperidyl]methanone |
| 429 | 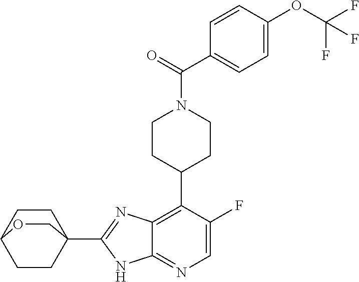 [SMILES: O=C(c1ccc(OC(F)(F)F)cc1)N1CCC(c2c(F)cnc3[nH]c(C45CCC(CC4)OC5)nc23)CC1] | [4-[6-fluoro-2-(2- oxabicyclo[2.2.2]octan-4-yl)-3H- imidazo[4,5-b]pyridin-7-yl]-1- piperidyl]-[4- (trifluoromethoxy)phenyl]methanone |
| 430 | 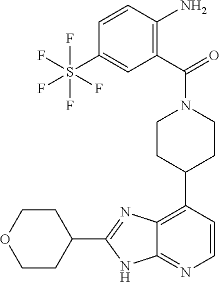 [SMILES: Nc1ccc(S(F)(F)(F)(F)F)cc1C(=O)N1CCC(c2ccnc3[nH]c(C4CCOCC4)nc23)CC1] | [2-amino-5-(pentafluoro-λ⁶- sulfanyl)phenyl]-[4-(2- tetrahydropyran-4-yl-3H- imidazo[4,5-b]pyridin-7-yl)-1- piperidyl]methanone |
| 431 | 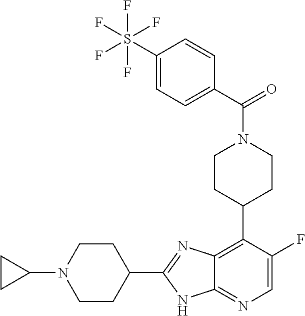 [SMILES: O=C(c1ccc(S(F)(F)(F)(F)F)cc1)N1CCC(c2c(F)cnc3[nH]c(C4CCN(C5CC5)CC4)nc23)CC1] | [4-[2-(1-cyclopropyl-4-piperidyl)-6- fluoro-3H-imidazo[4,5-b]pyridin-7- yl]-1-piperidyl]-[4-(pentafluoro-λ⁶- sulfanyl)phenyl]methanone |
| 432 | 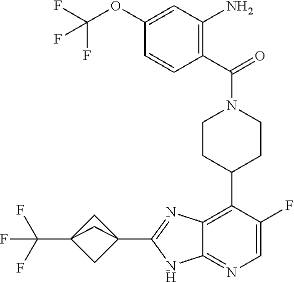 [SMILES: Nc1cc(OC(F)(F)F)ccc1C(=O)N1CCC(c2c(F)cnc3[nH]c(C45CC(C(F)(F)F)(C4)C5)nc23)CC1] | [2-amino-4- (trifluoromethoxy)phenyl]-[4-[6- fluoro-2-[3-(trifluoromethyl)-1- bicyclo[1.1.1 ]pentanyl]-3H- imidazo[4,5-b]pyridin-7-yl]-1- piperidyl]methanone |
| 433 | 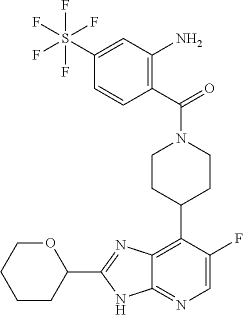 [SMILES: Nc1cc(S(F)(F)(F)(F)F)ccc1C(=O)N1CCC(c2c(F)cnc3[nH]c(C4CCCCO4)nc23)CC1] Isomer 1 | [2-amino-4-(pentafluoro-λ⁶- sulfanyl)phenyl]-[4-[6-fluoro-2- [tetrahydropyran-2-yl]-3H- imidazo[4,5-b]pyridin-7-yl]-1- piperidyl]methanone, Isomer 1 |
| 434 | 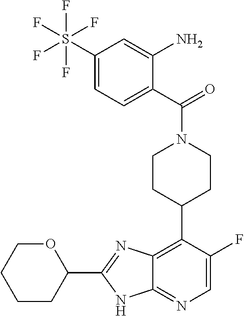 [SMILES: Nc1cc(S(F)(F)(F)(F)F)ccc1C(=O)N1CCC(c2c(F)cnc3[nH]c(C4CCCCO4)nc23)CC1] Isomer 2 | [2-amino-4-(pentafluoro-λ⁶- sulfanyl)phenyl]-[4-[6-fluoro-2- [tetrahydropyran-2-yl]-3H- imidazo[4,5-b]pyridin-7-yl]-1- piperidyl]methanone, Isomer 2 |
| 435 | 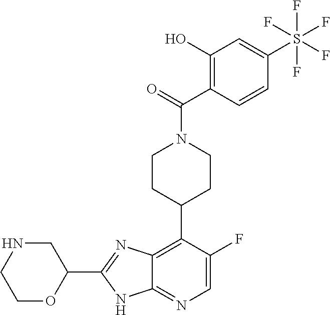 [SMILES: O=C(c1ccc(S(F)(F)(F)(F)F)cc1O)N1CCC(c2c(F)cnc3[nH]c(C4CNCCO4)nc23)CC1] | (rac)-[4-(6-fluoro-2-morpholin-2-yl- 3H-imidazo[4,5-b]pyridin-7-yl)-1- piperidyl]-[2-hydroxy-4- (pentafluoro-λ⁶- sulfanyl)phenyl]methanone |
| 436 | 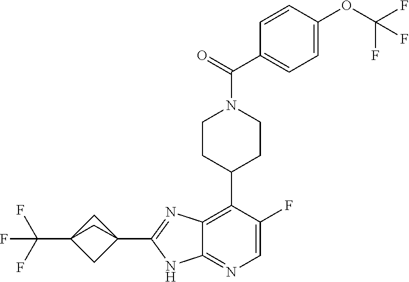 [SMILES: O=C(c1ccc(OC(F)(F)F)cc1)N1CCC(c2c(F)cnc3[nH]c(C45CC(C(F)(F)F)(C4)C5)nc23)CC1] | [4-[6-fluoro-2-[3-(trifluoromethyl)-1- bicyclo[1.1.1]pentanyl]-3H- imidazo[4,5-b]pyridin-7-yl]-1- piperidyl]-[4- (trifluoromethoxy)phenyl]methanone |
| 437 | 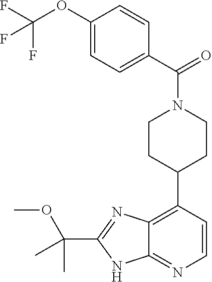 [SMILES: COC(C)(C)c1nc2c(C3CCN(C(=O)c4ccc(OC(F)(F)F)cc4)CC3)ccnc2[nH]1] | [4-[2-(1-methoxy-1-methyl-ethyl)- 3H-imidazo[4,5-b]pyridin-7-yl]-1- piperidyl]-[4- (trifluoromethoxy)phenyl]methanone |
| 438 | 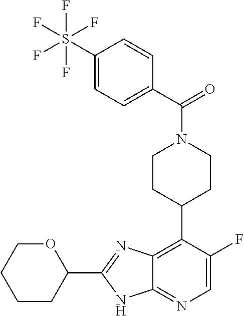 [SMILES: O=C(c1ccc(S(F)(F)(F)(F)F)cc1)N1CCC(c2c(F)cnc3[nH]c(C4CCCCO4)nc23)CC1] Isomer 1 | [4-[6-fluoro-2-[tetrahydropyran-2- yl]-3H-imidazo[4,5-b]pyridin-7-yl]- 1-piperidyl]-[4-(pentafluoro-λ⁶- sulfanyl)phenyl]methanone, Isomer 1 |
| 439 | 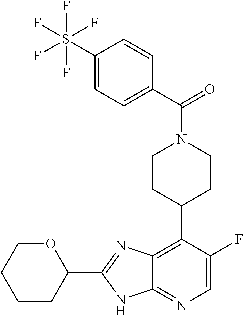 [SMILES: O=C(c1ccc(S(F)(F)(F)(F)F)cc1)N1CCC(c2c(F)cnc3[nH]c(C4CCCCO4)nc23)CC1] | [4-[6-fluoro-2-[tetrahydropyran-2- yl]-3H-imidazo[4,5-b]pyridin-7-yl]- 1-piperidyl]-[4-(pentafluoro-λ⁶- sulfanyl)phenyl]methanone, Isomer 2 |
| 440 | 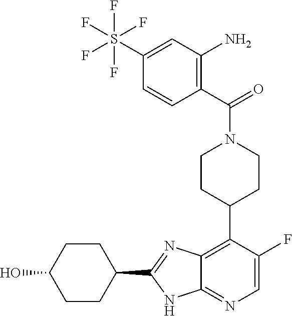 [SMILES: Nc1cc(S(F)(F)(F)(F)F)ccc1C(=O)N1CCC(c2c(F)cnc3[nH]c([C@H]4CC[C@H](O)CC4)nc23)CC1] Isomer 2 | (trans)-[2-amino-4-(pentafluoro-λ⁶- sulfanyl)phenyl]-[4-[6-fluoro-2-(4- hydroxycyclohexyl)-3H-imidazo[4,5- b]pyridin-7-yl]-1- piperidyl]methanone |
| 441 | 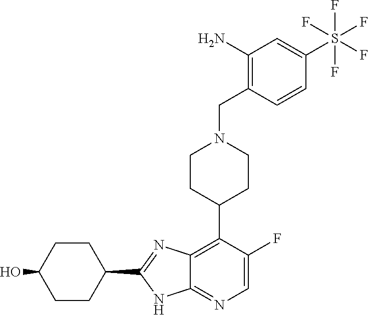 [SMILES: Nc1cc(S(F)(F)(F)(F)F)ccc1CN1CCC(c2c(F)cnc3[nH]c([C@H]4CC[C@@H](O)CC4)nc23)CC1] | (cis)-[2-amino-4-(pentafluoro-λ⁶- sulfanyl)phenyl]-[4-[6-fluoro-2-(4- hydroxycyclohexyl)-3H-imidazo[4,5- b]pyridin-7-yl]-1- piperidyl]methanone |
| 442 | 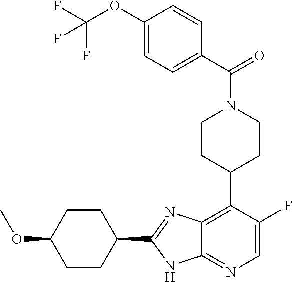 [SMILES: CO[C@H]1CC[C@@H](c2nc3c(C4CCN(C(=O)c5ccc(OC(F)(F)F)cc5)CC4)c(F)cnc3[nH]2)CC1] | (cis)-[4-[6-fluoro-2-(4- methoxycyclohexyl)-3H- imidazo[4,5-b]pyridin-7-yl]-1- piperidyl]-[4- (trifluoromethoxy)phenyl]methanone |
| 443 | 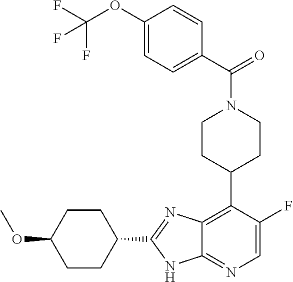 [SMILES: CO[C@H]1CC[C@H](c2nc3c(C4CCN(C(=O)c5ccc(OC(F)(F)F)cc5)CC4)c(F)cnc3[nH]2)CC1] | (trans)-[4-[6-fluoro-2-(4- methoxycyclohexyl)-3H- imidazo[4,5-b]pyridin-7-yl]-1- piperidyl]-[4- (trifluoromethoxy)phenyl]methanone |
| 444 | 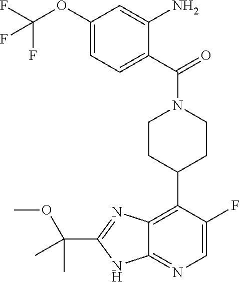 [SMILES: COC(C)(C)c1nc2c(C3CCN(C(=O)c4ccc(OC(F)(F)F)cc4N)CC3)c(F)cnc2[nH]1] | [4-[6-fluoro-2-(1-methoxy-1-methyl- ethyl)-3H-imidazo[4,5-b]pyridin-7- yl]-1-piperidyl]-[4- (trifluoromethoxy)phenyl]methanone |
| 445 | 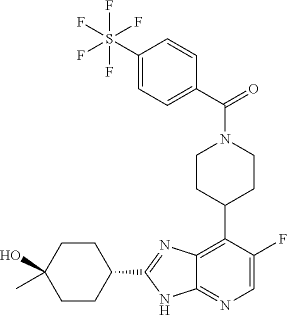 [SMILES: C[C@]1(O)CC[C@H](c2nc3c(C4CCN(C(=O)c5ccc(S(F)(F)(F)(F)F)cc5)CC4)c(F)cnc3[nH]2)CC1] | (trans)-(4-(6-fluoro-2-(4-hydroxy-4- methylcyclohexyl)-3H-imidazo[4,5- b]pyridin-7-yl)piperidin-1-yl)(4- (pentafluoro-λ⁶- sulfanyl)phenyl)methanone |
| 446 | 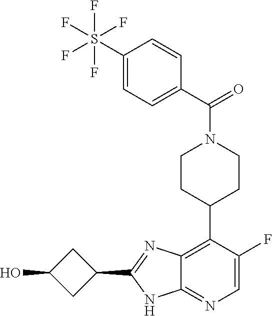 [SMILES: O=C(c1ccc(S(F)(F)(F)(F)F)cc1)N1CCC(c2c(F)cnc3[nH]c([C@H]4C[C@@H](O)C4)nc23)CC1] | (cis)-[4-[6-fluoro-2-(3- hydroxycyclobutyl)-3H-imidazo[4,5- b]pyridin-7-yl]-1-piperidyl]-[4- (pentafluoro-λ⁶- sulfanyl)phenyl]methanone |
| 447 | 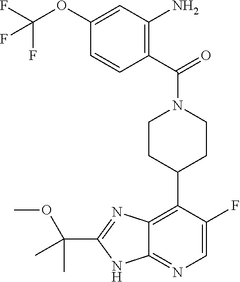 [SMILES: COC(C)(C)c1nc2c(C3CCN(C(=O)c4ccc(OC(F)(F)F)cc4N)CC3)c(F)cnc2[nH]1] | [2-amino-4- (trifluoromethoxy)phenyl]-[4-[6- fluoro-2-(1-methoxy-1-methyl- ethyl)-3H-imidazo[4,5-b]pyridin-7- yl]-1-piperidyl]methanone |
| 448 | 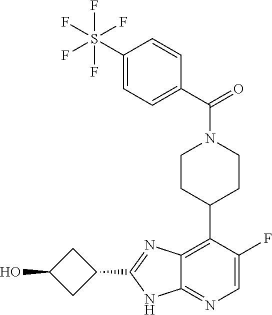 [SMILES: O=C(c1ccc(S(F)(F)(F)(F)F)cc1)N1CCC(c2c(F)cnc3[nH]c([C@H]4C[C@H](O)C4)nc23)CC1] | (trans)-[4-[6-fluoro-2-(3- hydroxycyclobutyl)-3H-imidazo[4,5- b]pyridin-7-yl]-1-piperidyl]-[4- (pentafluoro-λ⁶- sulfanyl)phenyl]methanone |
| 449 | 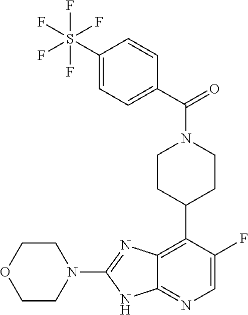 [SMILES: O=C(c1ccc(S(F)(F)(F)(F)F)cc1)N1CCC(c2c(F)cnc3[nH]c(N4CCOCC4)nc23)CC1] | 4-(6-fluoro-2-N-morpholino-3H- imidazo[4,5-b]pyridin-7-yl)-1- piperidyl]-[4-(pentafluoro-λ⁶- sulfanyl)phenyl]methanone |
| 450 | 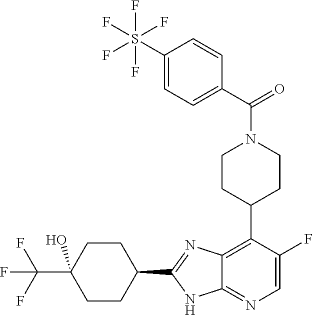 [SMILES: O=C(c1ccc(S(F)(F)(F)(F)F)cc1)N1CCC(c2c(F)cnc3[nH]c([C@H]4CC[C@@](O)(C(F)(F)F)CC4)nc23)CC1] | (trans)-[4-[6-fluoro-2-[4-hydroxy-4- (trifluoromethyl)cyclohexyl]-3H- imidazo[4,5-b]pyridin-7-yl]-1- piperidyl]-[4-(pentafluoro-λ⁶- sulfanyl)phenyl]methanone |
| 451 | 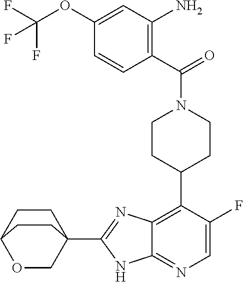 [SMILES: Nc1cc(OC(F)(F)F)ccc1C(=O)N1CCC(c2c(F)cnc3[nH]c(C45CCC(CC4)OC5)nc23)CC1] | [2-amino-4- (trifluoromethoxy)phenyl]-[4-[6- fluoro-2-(2-oxabicyclo[2.2.2]octan-4- yl)-3H-imidazo[4,5-b]pyridin-7-yl]- 1-piperidyl]methanone |
| 452 | 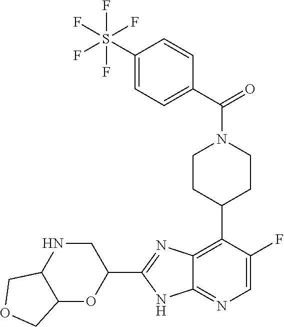 [SMILES: O=C(c1ccc(S(F)(F)(F)(F)F)cc1)N1CCC(c2c(F)cnc3[nH]c(C4CNC5COCC5O4)nc23)CC1] | (rac)-[4-[2-(3,4,4a,5,7,7a-hexahydro- 2H-furo[3,4-b][1,4]oxazin-2-yl)-6- fluoro-3H-imidazo[4,5-b]pyridin-7- yl]-1-piperidyl]-[4-(pentafluoro-λ⁶- sulfanyl)phenyl]methanone |
| 453 | 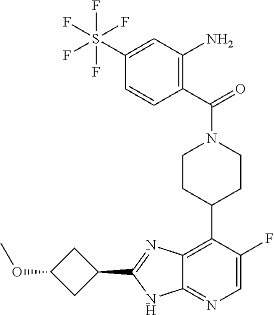 [SMILES: CO[C@H]1C[C@H](c2nc3c(C4CCN(C(=O)c5ccc(S(F)(F)(F)(F)F)cc5N)CC4)c(F)cnc3[nH]2)C1] | (trans)-[2-amino-4-(pentafluoro-λ⁶- sulfanyl)phenyl]-[4-[6-fluoro-2-(3- methoxycyclobutyl)-3H-imidazo[4,5- b]pyridin-7-yl]-1- piperidyl]methanone |
| 454 | 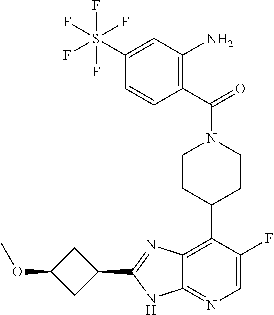 [SMILES: CO[C@H]1C[C@@H](c2nc3c(C4CCN(C(=O)c5ccc(S(F)(F)(F)(F)F)cc5N)CC4)c(F)cnc3[nH]2)C1] | (cis)-[2-amino-4-(pentafluoro-λ⁶- sulfanyl)phenyl]-[4-[6-fluoro-2-(3- methoxycyclobutyl)-3H-imidazo[4,5- b]pyridin-7-yl]-1- piperidyl]methanone |
| 455 | 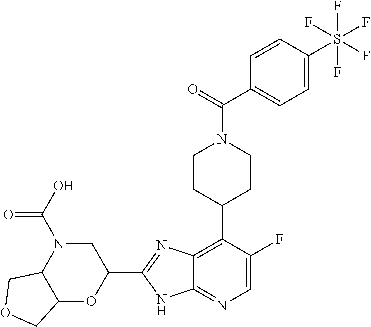 [SMILES: O=C(c1ccc(S(F)(F)(F)(F)F)cc1)N1CCC(c2c(F)cnc3[nH]c(C4CN(C(=O)O)C5COCC5O4)nc23)CC1] | (rac)-2-[6-fluoro-7-[1-[4- (pentafluoro-λ⁶-sulfanyl)benzoyl]-4- piperidyl]-3H-imidazo[4,5-b]pyridin- 2-yl]-2,3,4a,5,7,7a- hexahydrofuro[3,4-b][1,4]oxazine-4- carboxylic acid |
| 456 | 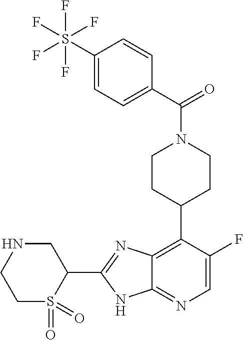 [SMILES: O=C(c1ccc(S(F)(F)(F)(F)F)cc1)N1CCC(c2c(F)cnc3[nH]c(C4CNCCS4(=O)=O)nc23)CC1] | (rac)-[4-[2-(1,1-dioxo-1,4-thiazinan- 2-yl)-6-fluoro-3H-imidazo[4,5- b]pyridin-7-yl]-1-piperidyl]-[4- (pentafluoro-λ⁶- sulfanyl)phenyl]methanone |
| 457 | 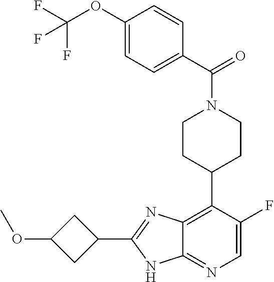 [SMILES: COC1CC(c2nc3c(C4CCN(C(=O)c5ccc(OC(F)(F)F)cc5)CC4)c(F)cnc3[nH]2)C1] | [4-[6-fluoro-2-(3- methoxycyclobutyl)-3H-imidazo[4,5- b]pyridin-7-yl]-1-piperidyl]-[4- (trifluoromethoxy)phenyl]methanone |
| 458 | 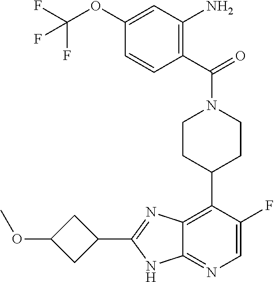 [SMILES: COC1CC(c2nc3c(C4CCN(C(=O)c5ccc(OC(F)(F)F)cc5N)CC4)c(F)cnc3[nH]2)C1] | (2-amino-4-(trifluoromethoxy) phenyl]-[4-[6-fluoro-2-(3- methoxycyclobutyl)-3H-imidazo[4,5- b]pyridin-7-yl]-1- piperidyl]methanone |
| 459 | 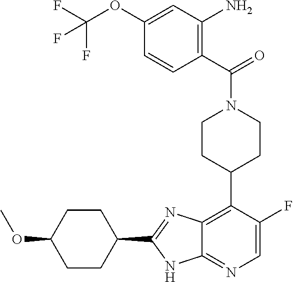 [SMILES: CO[C@H]1CC[C@@H](c2nc3c(C4CCN(C(=O)c5ccc(OC(F)(F)F)cc5N)CC4)c(F)cnc3[nH]2)CC1] | (cis)-[2-amino-4- (trifluoromethoxy)phenyl]-[4-[6- fluoro-2-(4-methoxycyclohexyl)-3H- imidazo[4,5-b]pyridin-7-yl]-1- piperidyl]methanone |
| 460 | 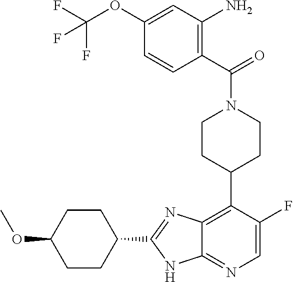 [SMILES: CO[C@H]1CC[C@H](c2nc3c(C4CCN(C(=O)c5ccc(OC(F)(F)F)cc5N)CC4)c(F)cnc3[nH]2)CC1] | (trans)-[2-amino-4- (trifluoromethoxy)phenyl]-[4-[6- fluoro-2-(4-methoxycyclohexyl)-3H- imidazo[4,5-b]pyridin-7-yl]-1- piperidyl]methanone |
| 461 | 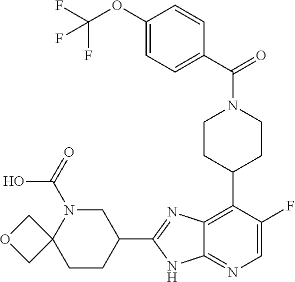 [SMILES: O=C(c1ccc(OC(F)(F)F)cc1)N1CCC(c2c(F)cnc3[nH]c(C4CCC5(COC5)N(C(=O)O)C4)nc23)CC1] | (rac)-7-[6-fluoro-7-[1-[4- (trifluoromethoxy)benzoyl]-4- piperidyl]-3H-imidazo[4,5-b]pyridin- 2-yl]-2-oxa-5-azaspiro[3.5]nonane-5- carboxylic acid |

### Examples 1 and 2: [2-Amino-4-(trifluoromethoxy)phenyl]-[4-[2-(tetrahydrofuran-3-ylmethyl)-3H-imidazo[4,5-b]pyridin-7-yl]-1-piperidyl]methanone, Isomers 1 and 2

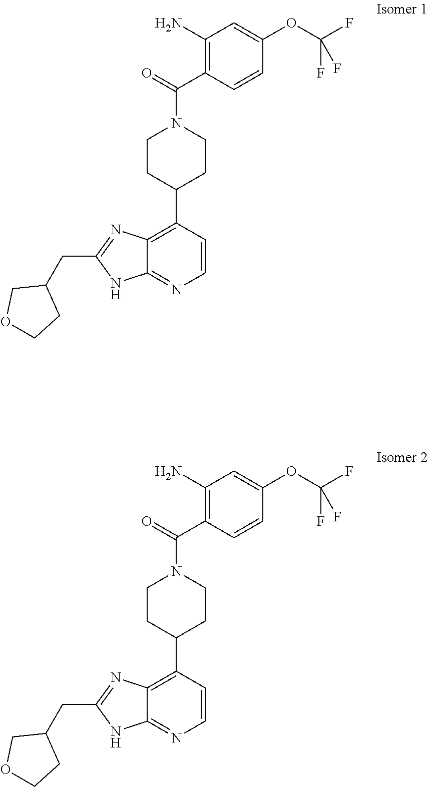 [SMILES: Nc1cc(OC(F)(F)F)ccc1C(=O)N1CCC(c2ccnc3[nH]c(CC4CCOC4)nc23)CC1 ; Nc1cc(OC(F)(F)F)ccc1C(=O)N1CCC(c2ccnc3[nH]c(CC4CCOC4)nc23)CC1]

### Step 1: Tert-butyl 4-(2-amino-3-nitro-4-pyridyl)-3,6-dihydro-2H-pyridine-1-carboxylate

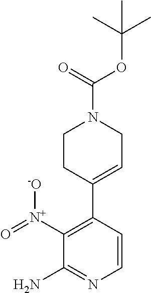

In a 2 litre three necked flask equipped with a reflux condenser, a thermometer and a bubbler, a solution of 4-chloro-3-nitro-pyridin-2-amine (20 g, 115.2 mmol), K₃PO₄ (73.38 g, 345.7 mmol), N-BOC-1,2,5,6-tetrahydropyridine-4-boronic acid pinacol ester (42.76 g, 138.3 mmol) in a mixture of 1,4-dioxane (1000 mL) and water (112 mL) was bubbled with argon for 15 minutes. Then, 1,1′-bis(diphenylphosphino)ferrocene-palladium(II)dichloride dichloromethane complex (4.71 g, 5.76 mmol) was added, and the resulting reaction mixture was stirred at 90° C. under argon atmosphere for 12 hours. The reaction mixture was cooled down to room temperature and 350 mL of AcOEt, 350 mL of water, were added. The organic phase was separated and the aqueous phase was extracted three times with AcOEt (3×350 mL). The combined organic layers were then washed twice with water (2×350 mL) and then with brine (350 mL), dried over magnesium sulfate and concentrated in vacuo. The resulting crude residue (85 g of a brown oil) was purified by flash chromatography (SiO₂ 600 g, eluting with heptane 75/AcOEt 25 at 100 mL/minute) to give 16.02 g (43%) of tert-butyl 4-(2-amino-3-nitro-4-pyridyl)-3,6-dihydro-2H-pyridine-1-carboxylate as a yellow solid. LC/MS (m/z, M+H): 321

### Step 2: Tert-butyl 4-(2,3-diamino-4-pyridyl)piperidine-1-carboxylate

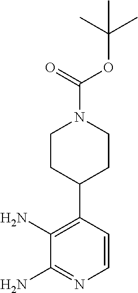 [SMILES: CC(C)(C)OC(=O)N1CCC(c2ccnc(N)c2N)CC1]

In a high pressure reactor was placed tert-butyl 4-(2-amino-3-nitro-4-pyridyl)-3,6-dihydro-2H-pyridine-1-carboxylate (16 g, 49.95 mmol) and Pd/C 10% (2.66 g, 2.5 mmol) in MeOH (637 mL). This mixture was then submitted to hydrogenation with 5 bars of hydrogen at 40° C. for 48 hours. The reaction mixture was then cooled down to room temperature, filtered on Clarcel®, washed with MeOH three times (3×50 mL). The resulting filtrate was concentrated in vacuo to furnish 14.92 g of a brown wax which was subsequently triturated with 10 mL of Et₂O+3 mL of iPr₂O. The resulting solid was filtered to give 10.95 g (75%) of tert-butyl 4-(2,3-diamino-4-pyridyl)piperidine-1-carboxylate as a brown solid. LC/MS (m/z, M+H): 293

### Step 3: (Rac)-tert-butyl 4-[2-(tetrahydrofuran-3-ylmethyl)-3H-imidazo[4,5-b]pyridin-7-yl]piperidine-1-carboxylate

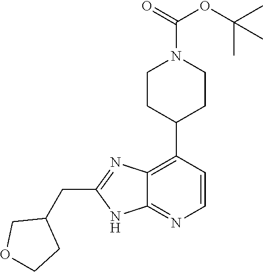 [SMILES: CC(C)(C)OC(=O)N1CCC(c2ccnc3[nH]c(CC4CCOC4)nc23)CC1]

To a solution of 2-tetrahydrofuran-3-ylacetic acid (111 mg, 0.85 mmol) in acetonitrile (9 mL) at room temperature under argon atmosphere was added CDI (166 mg, 1.02 mmol). The resulting mixture was stirred at room temperature for 20 minutes and tert-butyl 4-(2,3-diamino-4-pyridyl)piperidine-1-carboxylate (250 mg, 0.85 mmol) was added. The resulting mixture was then refluxed for 36 hours under argon atmosphere. The reaction mixture was then concentrated in vacuo, diluted with AcOEt (50 mL), transferred in a separating funnel, washed with water (50 mL) and with a saturated aqueous solution of sodium chloride (50 mL). The organic layer was dried over sodium sulfate, filtered and concentrated in vacuo. The resulting residue (black oil) was purified by flash chromatography (SiO₂ 12 g eluting with DCM 93/MeOH 7/NH₄OH 0.7) to give a brown solid which was subsequently triturated in iPr₂O, filtered and dried under vacuum to give 143 mg (43%) of tert-butyl 4-[2-(tetrahydrofuran-3-ylmethyl)-3H-imidazo[4,5-b]pyridin-7-yl]piperidine-1-carboxylate as a pale brown solid. LC/MS (m/z, M+H): 387

### Step 4: (Rac)-7-(4-piperidyl)-2-(tetrahydrofuran-3-ylmethyl)-3H-imidazo[4,5-b]pyridine, dihydrochloride

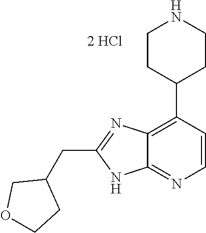 [SMILES: c1cc(C2CCNCC2)c2nc(CC3CCOC3)[nH]c2n1]

To a solution of tert-butyl 4-[2-(tetrahydrofuran-3-ylmethyl)-3H-imidazo[4,5-b]pyridin-7-yl]piperidine-1-carboxylate (140 mg, 0.36 mmol) in MeOH (2 mL) at room temperature was added HCl 4N solution in dioxane (0.91 mL, 3.62 mmol). The resulting mixture was stirred at room temperature for 2 hours. The reaction mixture was then concentrated and dried under vacuum to give 137 mg (100%) of (rac)-7-(4-piperidyl)-2-(tetrahydrofuran-3-ylmethyl)-3H-imidazo[4,5-b]pyridine dihydrochloride as a pale brown solid. LC/MS (m/z, M+H): 360 (free base)

### Step 5: (Rac)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone

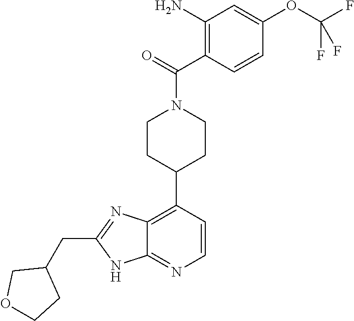 [SMILES: Nc1cc(OC(F)(F)F)ccc1C(=O)N1CCC(c2ccnc3[nH]c(CC4CCOC4)nc23)CC1]

To a solution of (rac)-7-(4-piperidyl)-2-(tetrahydrofuran-3-ylmethyl)-3H-imidazo[4,5-b]pyridine dihydrochloride (130 mg, 0.36 mmol) in DMF (2.4 mL) at room temperature under argon atmosphere was added DIPEA (0.32 mL, 1.81 mmol). After solubilization of the starting material, 2-amino-4-(trifluoromethoxy)benzoic acid (88 mg, 0.4 mmol) was added followed by HATU (165 mg, 0.43 mmol). The resulting mixture was then stirred at room temperature for 2 hours. The reaction mixture was diluted with AcOEt (40 mL), transferred in a separating funnel, washed with an aqueous saturated solution of NaHCO₃, then with water. The organic layer was then dried over sodium sulfate, filtered and concentrated in vacuo. The resulting residue (brown oil) was purified by flash chromatography (SiO₂ 12 g eluting with DCM 93/MeOH 7/NH₄OH 0.7) to give 106 mg (59%) of (rac)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone as a pale yellow solid. LC/MS (m/z, M+H): 490

Chiral separation of (rac)-[2-amino-4-(trifluoromethoxy)phenyl]-[4-[2-(tetrahydrofuran-3-ylmethyl)-3H-imidazo[4,5-b]pyridin-7-yl]-1-piperidyl]methanone (83 mg, 0.17 mmol), using a chiralpak AY column (20 μm, 100×230 mm), eluting with (n-Heptane 80% EtOH 15% MeOH 5)+0.1% TEA (flow rate 40 mL/min, UV detection at 280 nm) gave 37 mg (44%) of the first eluting Isomer 1 (Example 1) and 40 mg (48%) of Isomer 2 (Example 2) as white solids.

Isomer 1 (Example 1): LC/MS (m/z, M+H, Method 2): calc. 490, found 490.3; ¹H NMR (400 MHz, DMSO-d6, 30° C., mixture of conformer 70/30) 6 ppm 1.54-1.70 (m, 1H), 1.70-1.95 (m, 4H), 1.95-2.09 (m, 1H), 2.69-2.81 (m, 1H), 2.84-3.22 (m, 2H), 2.89 (br d, J=7 Hz, 2H), 3.18-3.40 (m partially hidden, 1H), 3.39-3.53 (m, 2H), 3.66 (q, J=8 Hz, 1H), 3.74-3.85 (m, 2H), 3.89-4.65 (m, 1H), 5.58 (br s, 2H), 6.49 (br d, J=8 Hz, 1H), 6.66 (br d, J=1 Hz, 1H), 7.05 (d, J=5 Hz, 1H), 7.15 (br d, J=8 Hz, 1H), 8.14 (br d, J=5 Hz, 0.7H), 8.18-8.35 (m, 0.3H), 12.49 (br s, 0.3H), 12.80 (br s, 0.7H)

Isomer 2 (Example 2): LC/MS (m/z, M+H, Method 2): calc. 490, found 490.3; 1H NMR (400 MHz, DMSO-d6, 30° C., mixture of conformer 70/30) δ ppm 1.57-1.69 (m, 1H), 1.70-1.94 (m, 4H), 1.95-2.09 (m, 1H), 2.69-2.81 (m, 1H), 2.85-2.96 (m, 2H), 2.95-3.40 (m partially hidden, 3H), 3.39-3.52 (m, 2H), 3.58-3.71 (m, 1H), 3.73-3.87 (m, 2H), 5.58 (s, 1.4H), 5.61 (s, 0.6H), 6.49 (br d, J=8 Hz, 1H), 6.66 (br s, 1H), 7.02-7.08 (m, 1H), 7.12-7.22 (m, 1H), 8.14 (d, J=5 Hz, 0.7H), 8.23 (d, J=5 Hz, 0.3H), 12.47 (s, 0.3H), 12.78 (br s, 0.7H)

### Examples 3 and 4: [2-Amino-4-(trifluoromethoxy)phenyl]-[4-(2-tetrahydrofuran-3-yl-3H-imidazo[4,5-b]pyridin-7-yl)-1-piperidyl]methanone, Isomers 1 and 2

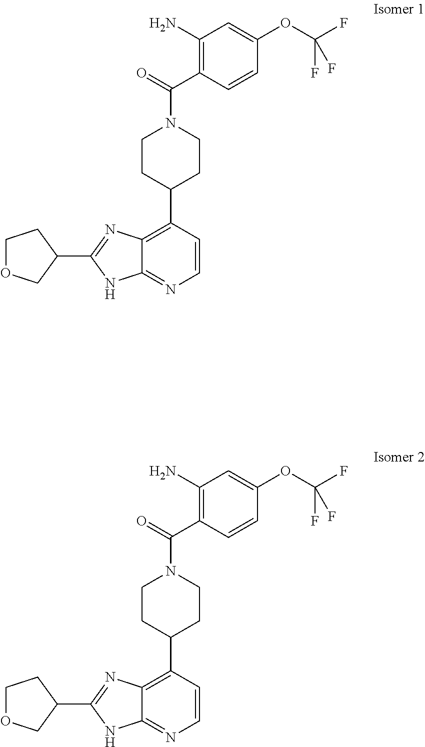 [SMILES: Nc1cc(OC(F)(F)F)ccc1C(=O)N1CCC(c2ccnc3[nH]c(C4CCOC4)nc23)CC1 ; Nc1cc(OC(F)(F)F)ccc1C(=O)N1CCC(c2ccnc3[nH]c(C4CCOC4)nc23)CC1]

### Step 1: (Rac)-tert-butyl 4-(2-tetrahydrofuran-3-yl-3H-imidazo[4,5-b]pyridin-7-yl)piperidine-1-carboxylate

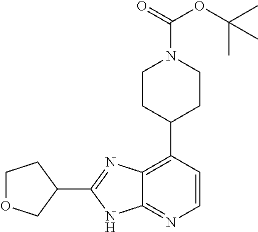 [SMILES: CC(C)(C)OC(=O)N1CCC(c2ccnc3[nH]c(C4CCOC4)nc23)CC1]

Step 1 of Examples 3 and 4 was prepared following a similar procedure to that of step 3 of Examples 1 and 2 from tert-butyl 4-(2,3-diamino-4-pyridyl)piperidine-1-carboxylate (250 mg, 0.86 mmol) and tetrahydrofuran-3-carboxylic acid (99 mg, 0.85 mmol) in acetonitrile (8.6 mL) to give 144 mg (45%) of (rac)-tert-butyl 4-(2-tetrahydrofuran-3-yl-3H-imidazo[4,5-b]pyridin-7-yl)piperidine-1-carboxylate as a pale brown solid. LC/MS (m/z, M+H): 373

### Step 2: (Rac)-7-(4-piperidyl)-2-tetrahydrofuran-3-yl-3H-imidazo[4,5-b]pyridine, dihydrochloride

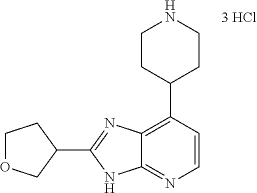 [SMILES: c1cc(C2CCNCC2)c2nc(C3CCOC3)[nH]c2n1]

Step 2 of Examples 3 and 4 was prepared following a similar procedure to that of step 4 of Examples 1 and 2 from (rac)-tert-butyl 4-(2-tetrahydrofuran-3-yl-3H-imidazo[4,5-b]pyridin-7-yl)piperidine-1-carboxylate (141 mg, 0.38 mmol) to give 130 mg (100%) of (rac)-7-(4-piperidyl)-2-tetrahydrofuran-3-yl-3H-imidazo[4,5-b]pyridine dihydrochloride as a pale brown solid. LC/MS (m/z, M+H): 273

### Step 3: (Rac)-[2-amino-4-(trifluoromethoxy)phenyl]-[4-(2-tetrahydrofuran-3-yl-3H-imidazo[4,5-b]pyridin-7-yl)-1-piperidyl]methanone

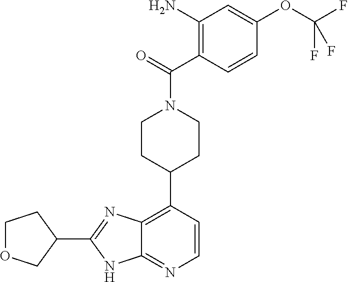 [SMILES: Nc1cc(OC(F)(F)F)ccc1C(=O)N1CCC(c2ccnc3[nH]c(C4CCOC4)nc23)CC1]

Step 3 of Examples 3 and 4 was prepared following a similar procedure to that of step 5 of Examples 1 and 2 from (rac)-7-(4-piperidyl)-2-tetrahydrofuran-3-yl-3H-imidazo[4,5-b]pyridine dihydrochloride (130 mg, 0.38 mmol) to give 118 mg (64%) of (rac)-[2-amino-4-(trifluoromethoxy)phenyl]-[4-(2-tetrahydrofuran-3-yl-3H-imidazo[4,5-b]pyridin-7-yl)-1-piperidyl]methanone as a pale yellow solid. LC/MS (m/z, M+H): 476

Chiral separation of (rac)-[2-amino-4-(trifluoromethoxy)phenyl]-[4-(2-tetrahydrofuran-3-yl-3H-imidazo[4,5-b]pyridin-7-yl)-1-piperidyl]methanone (93 mg, 0.19 mmol), using a chiralpak AY column (20 μm, 100×230 mm), eluting with (n-Heptane 70% EtOH 25% MeOH 5%)+0.1% TEA (flow rate 40 mL/min, UV detection at 265 nm) gave 42 mg (45%) of the first eluting Isomer 1 (Example 3) and 48 mg (52%) of Isomer 2 (Example 4) as white solids.

Isomer 1 (Example 3): LC/MS (m/z, M+H, Method 2): calc. 476, found 476.1; ¹H NMR (400 MHz, DMSO-d6, 100° C.) δ ppm 1.82-1.99 (m, 4H), 2.34 (q, J=7 Hz, 2H), 3.04-3.17 (m, 2H), 3.35-3.49 (m, 1H), 3.67 (quin, J=8 Hz, 1H), 3.83 (q, J=7 Hz, 1H), 3.89-4.01 (m, 2H), 4.07-4.12 (m, 1H), 4.16 (br d, J=13 Hz, 2H), 5.34 (s, 2H), 6.48 (br dd, J=8, 1 Hz, 1H), 6.68 (br d, J=1 Hz, 1H), 7.01 (d, J=5 Hz, 1H), 7.14 (d, J=9 Hz, 1H), 8.15 (d, J=5 Hz, 1H), 11.88-12.82 (m, 1H)

Isomer 2 (Example 4): LC/MS (m/z, M+H, Method 2): calc. 476, found 476.1; ¹H NMR (400 MHz, DMSO-d6, 100° C.) δ ppm 1.82-1.99 (m, 4H), 2.34 (q, J=7 Hz, 2H), 3.04-3.17 (m, 2H), 3.35-3.49 (m, 1H), 3.67 (quin, J=8 Hz, 1H), 3.83 (q, J=7 Hz, 1H), 3.89-4.01 (m, 2H), 4.07-4.12 (m, 1H), 4.16 (br d, J=13 Hz, 2H), 5.34 (s, 2H), 6.48 (br dd, J=8, 1 Hz, 1H), 6.68 (br d, J=1 Hz, 1H), 7.01 (d, J=5 Hz, 1H), 7.14 (d, J=9 Hz, 1H), 8.15 (d, J=5 Hz, 1H), 11.88-12.82 (m, 1H)

### Example 5: [2-Amino-4-(trifluoromethoxy)phenyl]-[4-(2-tetrahydropyran-4-yl-3H-imidazo[4,5-b]pyridin-7-yl)-1-piperidyl]methanone

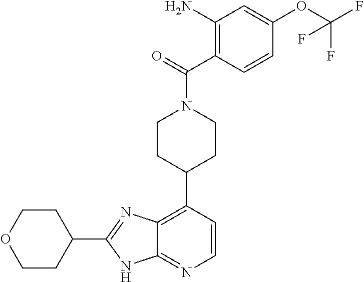 [SMILES: Nc1cc(OC(F)(F)F)ccc1C(=O)N1CCC(c2ccnc3[nH]c(C4CCOCC4)nc23)CC1]

### Step 1: Tert-butyl 4-(2-tetrahydropyran-4-yl-3H-imidazo[4,5-b]pyridin-7-yl)piperidine-1-carboxylate

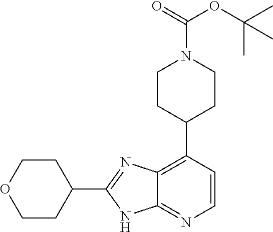 [SMILES: CC(C)(C)OC(=O)N1CCC(c2ccnc3[nH]c(C4CCOCC4)nc23)CC1]

To a solution of tetrahydropyran-4-carboxylic acid (44 mg, 0.34 mmol) in acetonitrile (3.4 mL) at room temperature was added CDI (66 mg, 0.41 mmol) and the reaction mixture was stirred at room temperature for one hour. Then tert-butyl 4-(2,3-diamino-4-pyridyl)piperidine-1-carboxylate (100 mg, 0.34 mmol) was added and the resulting reaction mixture was refluxed for 36 hours. The reaction mixture was cooled down to room temperature. The formed precipitate was filtered, washed with acetonitrile and dried under vacuum to give 76 mg (57%) of tert-butyl 4-(2-tetrahydropyran-4-yl-3H-imidazo[4,5-b]pyridin-7-yl)piperidine-1-carboxylate as a pale brown solid. LC/MS (m/z, M+H): 387

### Step 2: [2-Amino-4-(trifluoromethoxy)phenyl]-[4-(2-tetrahydropyran-4-yl-3H-imidazo[4,5-b]pyridin-7-yl)-1-piperidyl]methanone

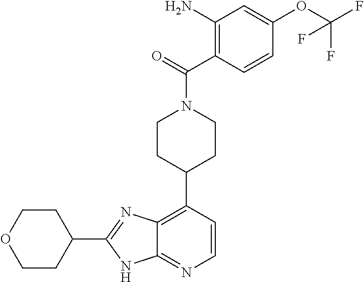 [SMILES: Nc1cc(OC(F)(F)F)ccc1C(=O)N1CCC(c2ccnc3[nH]c(C4CCOCC4)nc23)CC1]

To a solution of tert-butyl 4-(2-tetrahydropyran-4-yl-3H-imidazo[4,5-b]pyridin-7-yl)piperidine-1-carboxylate (69 mg, 0.18 mmol) in DCM (1.7 mL) at room temperature under argon atmosphere was added TFA (0.23 mL, 2.87 mmol) and the reaction mixture was stirred at room temperature for 3 hours. The reaction mixture was then concentrated in vacuo to dryness. The resulting residue was then diluted with DMF (1.2 mL), and under argon atmosphere, was added DIEA (0.37 mL, 2.14 mmol) followed 5 minutes later by HATU (68 mg, 0.18 mmol) portionwise. The resulting mixture was then stirred at room temperature for 3.5 hours. The reaction mixture was then diluted with water (15 mL) and with a 1N aqueous solution of NaOH (15 mL), transferred in a separating funnel, extracted three times with AcOEt. The organic layers were combined, washed with a saturated aqueous solution of sodium chloride, dried over sodium sulfate and concentrated in vacuo. The resulting residue was suspended in a minimum of DCM, 3 drops of MeOH were added to solubilize and then pentane was added progressively until a white precipitate appeared which was filtered, washed with pentane and dried under vacuum to give 65 mg (74%) of [2-amino-4-(trifluoromethoxy)phenyl]-[4-(2-tetrahydropyran-4-yl-3H-imidazo[4,5-b]pyridin-7-yl)-1-piperidyl]methanone as a pale yellow solid. LC/MS (m/z, M+H, Method 2): calc. 490, found 490.2; ¹H NMR (400 MHz, DMSO-d6, 101° C.) δ ppm 1.75-2.02 (m, 8H), 3.00-3.23 (m, 3H), 3.31-3.47 (m, 1H), 3.44-3.56 (m, 2H), 3.95 (dt, J=11, 3 Hz, 2H), 4.17 (br d, J=13 Hz, 2H), 5.33 (s, 2H), 6.48 (dd, J=8, 1 Hz, 1H), 6.68 (d, J=1 Hz, 1H), 7.00 (d, J=5 Hz, 1H), 7.15 (d, J=8 Hz, 1H), 8.14 (br d, J=5 Hz, 1H), 12-12.6 (m, 1H)

### Example 6: [4-[2-(4-Piperidyl)-3H-imidazo[4,5-b]pyridin-7-yl]-1-piperidyl]-[4-(trifluoromethoxy)phenyl]methanone

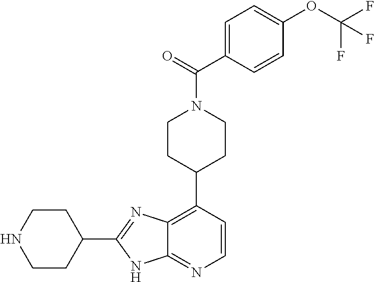 [SMILES: O=C(c1ccc(OC(F)(F)F)cc1)N1CCC(c2ccnc3[nH]c(C4CCNCC4)nc23)CC1]

### Step 1: [4-(2,3-Diamino-4-pyridyl)-1-piperidyl]-[4-(trifluoromethoxy)phenyl]methanone

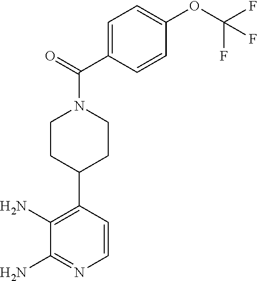 [SMILES: Nc1nccc(C2CCN(C(=O)c3ccc(OC(F)(F)F)cc3)CC2)c1N]

To a solution of tert-butyl 4-(2,3-diamino-4-pyridyl)piperidine-1-carboxylate (1.8 g, 6.2 mmol) in DCM (10 mL) at 0° C. under argon atmosphere was dropwise added TFA (4.7 mL, 62 mmol). The reaction mixture was then stirred at room temperature for 3.5 hours. The reaction mixture was then concentrated to dryness and the resulting residue was diluted with DMF (20 mL) under argon atmosphere, and at 0° C., DIEA (11 mL, 62 mmol) was added. The resulting mixture was stirred for 10 minutes, and 4-(trifluoromethoxy)benzoic acid (1.3 g, 62 mmol) was added followed by HATU (2.3 g, 6.2 mmol) portionwise. The reaction mixture was stirred at room temperature for 12 hours, then diluted with 80 mL of water, transferred in a separating funnel, extracted three times with AcOEt (3×30 mL). The combined organic layers were washed with a 1 N NaOH solution, with brine, dried over sodium sulfate, filtered and concentrated in vacuo. The resulting residue was purified by flash chromatography (SiO₂ 120 g eluting with DCM 90/MeOH 10) to give a brown solid which was then suspended in a minimum of DCM. Three drop of MeOH were added to solubilize and pentane was progressively added until a precipitate appeared. This solid was filtered, washed with pentane and dried under vacuum to give 1.33 g (57%) of [4-(2,3-diamino-4-pyridyl)-1-piperidyl]-[4-(trifluoromethoxy)phenyl]methanone as a pale brown solid. LC/MS (m/z, M+H): 381

### Step 2: Tert-butyl 4-[7-[1-[4-(trifluoromethoxy)benzoyl]-4-piperidyl]-3H-imidazo[4,5-b]pyridin-2-yl]piperidine-1-carboxylate

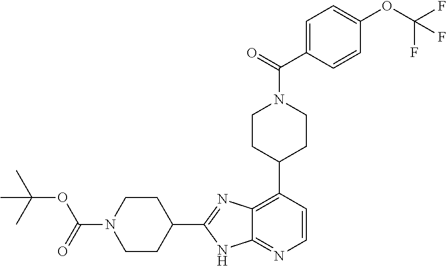 [SMILES: CC(C)(C)OC(=O)N1CCC(c2nc3c(C4CCN(C(=O)c5ccc(OC(F)(F)F)cc5)CC4)ccnc3[nH]2)CC1]

Step 2 of Example 6 was prepared following a similar procedure to that of step 1 of Example from [4-(2,3-diamino-4-pyridyl)-1-piperidyl]-[4-(trifluoromethoxy)phenyl]methanone (150 mg, 0.39 mmol) and 1-tert-butoxycarbonylpiperidine-4-carboxylic acid (92 mg, 0.39 mmol) to give 145 mg (64%) of tert-butyl 4-[7-[1-[4-(trifluoromethoxy)benzoyl]-4-piperidyl]-3H-imidazo[4,5-b]pyridin-2-yl]piperidine-1-carboxylate as a white solid. LC/MS (m/z, M+H): 574

### Step 3: [4-[2-(4-Piperidyl)-3H-imidazo[4,5-b]pyridin-7-yl]-1-piperidyl]-[4-(trifluoromethoxy)phenyl]methanone

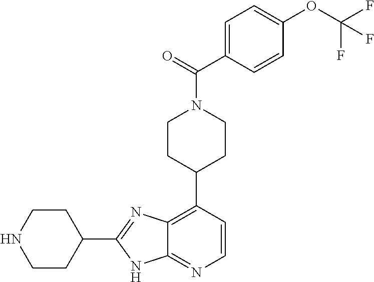 [SMILES: O=C(c1ccc(OC(F)(F)F)cc1)N1CCC(c2ccnc3[nH]c(C4CCNCC4)nc23)CC1]

To a solution of tert-butyl 4-[7-[1-[4-(trifluoromethoxy)benzoyl]-4-piperidyl]-3H-imidazo[4,5-b]pyridin-2-yl]piperidine-1-carboxylate (139 mg, 0.24 mmol) in DCM (0.5 mL) at room temperature under argon atmosphere was added TFA (0.22 mL, 2.87 mmol) and the resulting reaction mixture was stirred at room temperature for 12 hours. The reaction mixture was then concentrated in vacuo, diluted with AcOEt, washed with a 1 N NaOH solution. The aqueous phase was extracted twice with AcOEt. The combined organic layers were then washed with brine, dried over sodium sulfate, filtered and concentrated. The resulting residue was then purified by reverse phase HPLC with a 5 μm CSH column (250×50 mm) eluting with 94% of A (H₂O+0.1% formic acid) and 6% of B (acetonitrile+0.1% formic acid) at t=0 to 5 minutes, then gradually eluting up to 26% of B at t=25 minutes (flow rate: 150 mL/min; room temperature) to give 12 mg of [4-[2-(4-piperidyl)-3H-imidazo[4,5-b]pyridin-7-yl]-1-piperidyl]-[4-(trifluoromethoxy)phenyl]methanone as a white solid. LC/MS (m/z, M+H, Method 1): calc. 474, found 474.2; ¹H NMR (300 MHz, DMSO-d6, 120° C.) δ ppm 1.68-2.04 (m, 8H), 2.58-2.74 (m, 2H), 2.85-3.03 (m partially hidden, 1H), 3.03-3.21 (m, 4 H), 3.36-3.50 (m, 1H), 4.16 (br d, J=12 Hz, 2H), 6.99 (d, J=5 Hz, 1H), 7.37 (br d, J=9 Hz, 2H), 7.56 (d, J=9 Hz, 2H), 8.14 (d, J=5 Hz, 1H)

### Example 7: (Trans)-[4-[2-(4-hydroxycyclohexyl)-3H-imidazo[4,5-b]pyridin-7-yl]-1-piperidyl]-[4-(trifluoromethoxy)phenyl]methanone

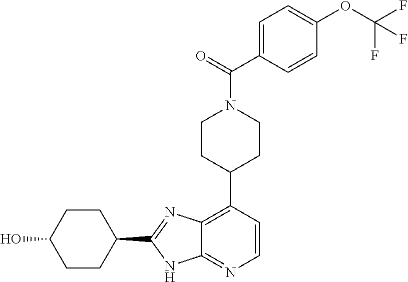 [SMILES: O=C(c1ccc(OC(F)(F)F)cc1)N1CCC(c2ccnc3[nH]c([C@H]4CC[C@H](O)CC4)nc23)CC1]

### Step 1: (Trans)-4-acetoxycyclohexanecarboxylic acid

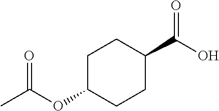

To a solution of (trans)-4-hydroxycyclohexanecarboxylic acid (500 mg, 3.29 mmol) in pyridine (5 mL) under argon atmosphere at 5° C. was dropwise added acetic anhydride (2.5 mL, 26 mmol). After 10 minutes, the reaction mixture was allowed to warm up to room temperature. After one hour, the reaction mixture was poured on crushed ice and acidified to pH=1 with HCl. The whole mixture was transferred in a separating funnel, extracted twice with AcOEt. The combined organic layers were then washed with brine, dried over sodium sulfate, filtered and concentrated in vacuo to give 613 mg (100%) of (trans)-4-acetoxycyclohexanecarboxylic acid as a white solid.

### Step 2: (Trans)-[4-[[3-amino-4-[1-[4-(trifluoromethoxy)benzoyl]-4-piperidyl]-2-pyridyl]carbamoyl]cyclohexyl]acetate

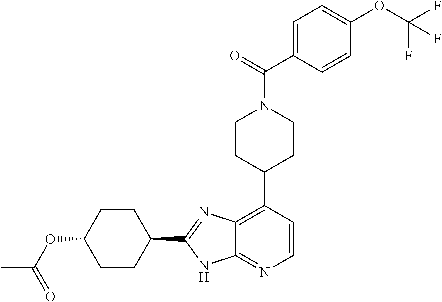 [SMILES: CC(=O)O[C@H]1CC[C@H](c2nc3c(C4CCN(C(=O)c5ccc(OC(F)(F)F)cc5)CC4)ccnc3[nH]2)CC1]

Step 2 of Example 7 was prepared following a similar procedure to that of step 1 of Example 5 from (trans)-[4-(2,3-diamino-4-pyridyl)-1-piperidyl]-[4-(trifluoromethoxy)phenyl]methanone (100 mg, 0.26 mmol) and 4-acetoxycyclohexanecarboxylic acid (52 mg, 0.28 mmol) to give 65 mg (47%) of (trans)-[4-[[3-amino-4-[1-[4-(trifluoromethoxy)benzoyl]-4-piperidyl]-2-pyridyl]carbamoyl]cyclohexyl]acetate as a white solid. LC/MS (m/z, M+H): 531

### Step 3: (Trans)-[4-[2-(4-hydroxycyclohexyl)-3H-imidazo[4,5-b]pyridin-7-yl]-1-piperidyl]-[4-(trifluoromethoxy)phenyl]methanone

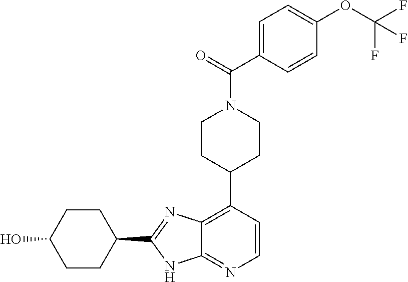 [SMILES: O=C(c1ccc(OC(F)(F)F)cc1)N1CCC(c2ccnc3[nH]c([C@H]4CC[C@H](O)CC4)nc23)CC1]

A solution of (trans)-4-[[3-amino-4-[1-[4-(trifluoromethoxy)benzoyl]-4-piperidyl]-2-pyridyl]carbamoyl]cyclohexyl]acetate (62 mg, 0.12 mmol) in a mixture of THF 0.5 mL/MeOH 0.5 mL/(NaOH aq 1N) 0.5 mL at room temperature was stirred for 12 hours. After 12 hours, the reaction mixture was concentrated in vacuo, diluted with water, transferred in a separating funnel and extracted twice with AcOEt. The combined organic layers were washed with brine, dried over sodium sulfate, filtered and concentrated in vacuo. The resulting residue was then triturated in Et₂O, filtered and dried under vacuum to give 46 (810%) mg of (trans)-[4-[2-(4-hydroxycyclohexyl)-3H-imidazo[4,5-b]pyridin-7-yl]-1-piperidyl]-[4-(trifluoromethoxy)phenyl]methanone as a white solid. LC/MS (m/z, M+H, Method 1): calc. 489, found 489.2; ¹H NMR (300 MHz, DMSO-d6, 120° C.) δ ppm 1.22-1.50 (m, 2H), 1.63-1.81 (m, 2H), 1.83-2.16 (m, 8H), 2.68-2.98 (m partially hidden, 1H), 3.08-3.24 (m, 2H), 3.35-3.70 (m, 2H), 4.10 (br d, J=3 Hz, 1H), 4.19 (br d, J=12 Hz, 2H), 7.02 (d, J=5 Hz, 1H), 7.40 (br d, J=8 Hz, 2H), 7.59 (br d, J=9 Hz, 2H), 8.16 (br d, J=5 Hz, 1H), 11.85-12.65 (m, 1H)

### Example 8: (Cis)-[4-[2-(4-hydroxycyclohexyl)-3H-imidazo[4,5-b]pyridin-7-yl]-1-piperidyl]-[4-(trifluoromethoxy)phenyl]methanone

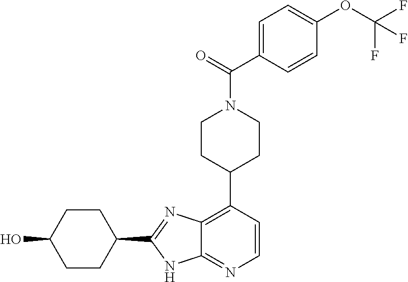 [SMILES: O=C(c1ccc(OC(F)(F)F)cc1)N1CCC(c2ccnc3[nH]c([C@H]4CC[C@@H](O)CC4)nc23)CC1]

### Step 1: (Cis)-4-acetoxycyclohexanecarboxylic acid

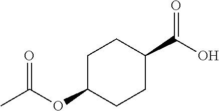

Step 1 of Example 8 was prepared following a similar procedure to that of step 1 of Example 7 from (cis)-4-hydroxycyclohexanecarboxylic acid (500 mg, 3.29 mmol) and acetic anhydride (2.5 mL, 26 mmol) to give 613 mg (100%) of (cis)-4-acetoxycyclohexanecarboxylic acid as a colorless crystalline solid. LC/MS (m/z, M+H):

### Step 2: (Cis)-[4-[7-[1-[4-(trifluoromethoxy)benzoyl]-4-piperidyl]-3H-imidazo[4,5-b]pyridin-2-yl]cyclohexyl]acetate

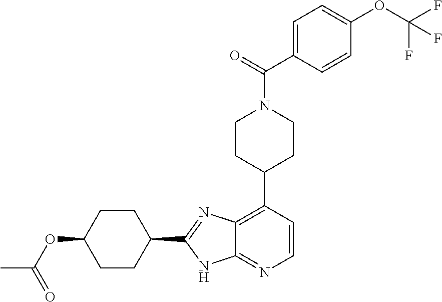 [SMILES: CC(=O)O[C@H]1CC[C@@H](c2nc3c(C4CCN(C(=O)c5ccc(OC(F)(F)F)cc5)CC4)ccnc3[nH]2)CC1]

Step 2 of Example 8 was prepared following a similar procedure to that of step 1 of Example from [4-(2,3-diamino-4-pyridyl)-1-piperidyl]-[4-(trifluoromethoxy)phenyl]methanone (100 mg, 0.26 mmol) and (cis)-4-acetoxycyclohexanecarboxylic acid (52 mg, 0.28 mmol) to give 70 mg (50%) of (cis)-[4-[7-[1-[4-(trifluoromethoxy)benzoyl]-4-piperidyl]-3H-imidazo[4,5-b]pyridin-2-yl]cyclohexyl]acetate as a white solid. LC/MS (m/z, M+H): 531

### Step 3: (Cis)-[4-[2-(4-hydroxycyclohexyl)-3H-imidazo[4,5-b]pyridin-7-yl]-1-piperidyl]-[4-(trifluoromethoxy)phenyl]methanone

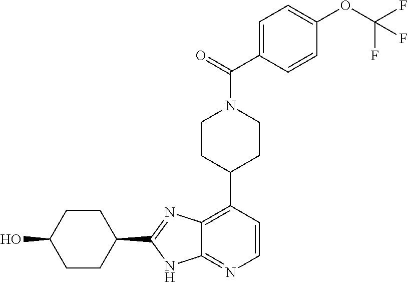 [SMILES: O=C(c1ccc(OC(F)(F)F)cc1)N1CCC(c2ccnc3[nH]c([C@H]4CC[C@@H](O)CC4)nc23)CC1]

Step 3 of Example 8 was prepared following a similar procedure to that of step 3 of Example 7 from (cis)-[4-[7-[1-[4-(trifluoromethoxy)benzoyl]-4-piperidyl]-3H-imidazo[4,5-b]pyridin-2-yl]cyclohexyl]acetate (70 mg, 0.13 mmol) to give 30 mg (46%) of (cis)-[4-[2-(4-hydroxycyclohexyl)-3H-imidazo[4,5-b]pyridin-7-yl]-1-piperidyl]-[4-(trifluoromethoxy)phenyl]methanone (6) as a white solid. LC/MS (m/z, M+H, Method 1): calc. 489, found 489.2; ¹H NMR (300 MHz, DMSO-d6, 120° C.) δ ppm 1.52-1.71 (m, 2H), 1.71-1.86 (m, 4H), 1.88-2.05 (m, 4H), 2.09-2.27 (m, 2H), 2.85-3.01 (m partially hidden, 1H), 3.08-3.25 (m, 2H), 3.36-3.57 (m, 1H), 3.80-3.98 (m, 2H), 4.20 (br d, J=12 Hz, 2H), 7.02 (d, J=5 Hz, 1H), 7.40 (br d, J=9 Hz, 2H), 7.60 (d, J=9 Hz, 2H), 8.16 (br d, J=4 Hz, 1H), 11.86-12.62 (m, 1H)

### Example 9: (Cis)-[4-[2-(4-aminocyclohexyl)-3H-imidazo[4,5-b]pyridin-7-yl]-1-piperidyl]-[4-(trifluoromethoxy)phenyl]methanone

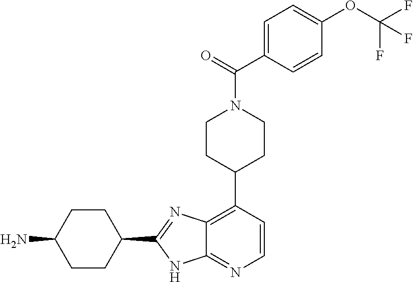 [SMILES: N[C@H]1CC[C@@H](c2nc3c(C4CCN(C(=O)c5ccc(OC(F)(F)F)cc5)CC4)ccnc3[nH]2)CC1]

### Step 1: Tert-butyl N-[4-[7-[1-[4-(trifluoromethoxy)benzoyl]-4-piperidyl]-3H-imidazo[4,5-b]pyridin-2-yl]cyclohexyl]carbamate

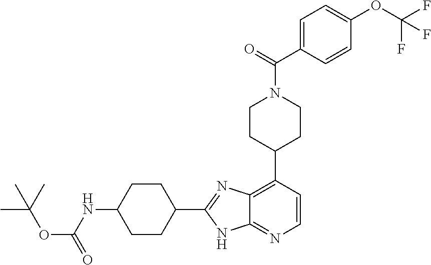 [SMILES: CC(C)(C)OC(=O)NC1CCC(c2nc3c(C4CCN(C(=O)c5ccc(OC(F)(F)F)cc5)CC4)ccnc3[nH]2)CC1]

Step 1 of Example 9 was prepared following a similar procedure to that of step 1 of Example from [4-(2,3-diamino-4-pyridyl)-1-piperidyl]-[4-(trifluoromethoxy)phenyl]methanone (150 mg, 0.39 mmol) and 4-(tert-butoxycarbonylamino)cyclohexanecarboxylic acid (98 mg, 0.39 mmol) to give 164 mg (71%) of tert-butyl N-[4-[7-[1-[4-(trifluoromethoxy)benzoyl]-4-piperidyl]-3H-imidazo[4,5-b]pyridin-2-yl]cyclohexyl]carbamate as a white solid. LC/MS (m/z, M+H): 588

### Step 2: (Cis)-[4-[2-(4-aminocyclohexyl)-3H-imidazo[4,5-b]pyridin-7-yl]-1-piperidyl]-[4-(trifluoromethoxy)phenyl]methanone

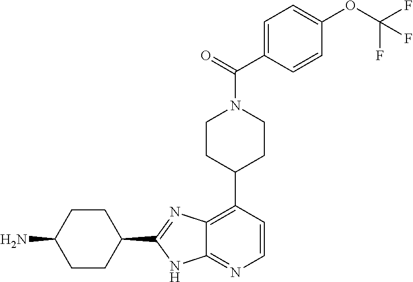 [SMILES: N[C@H]1CC[C@@H](c2nc3c(C4CCN(C(=O)c5ccc(OC(F)(F)F)cc5)CC4)ccnc3[nH]2)CC1]

To a solution of tert-butyl N-[4-[7-[1-[4-(trifluoromethoxy)benzoyl]-4-piperidyl]-3H-imidazo[4,5-b]pyridin-2-yl]cyclohexyl]carbamate (158 mg, 0.27 mmol) in DCM (0.5 mL) under at room temperature argon atmosphere was added TFA (0.12 mL, 1.61 mmol). After 6 hours, additional 0.1 mL of TFA were added, and the reaction mixture was then stirred 12 hours at room temperature. Then, the reaction mixture was concentrated in vacuo, diluted with water and AcOEt, basified with NaOH 1N to pH>10. The organic layer was separated and the aqueous layer was extracted twice with AcOEt. The combined organic layers were then washed with brine, dried over sodium sulfate, filtered and concentrated to give 148 mg of a brown wax. This residue was solubilized in a minimum of DCM and pentane was progressively added until a white precipitate appeared. The latter was filtered, washed with pentane and dried under vacuum to give 106 mg (81%) of (cis)-[4-[2-(4-aminocyclohexyl)-3H-imidazo[4,5-b]pyridin-7-yl]-1-piperidyl]-[4-(trifluoromethoxy)phenyl]methanone as a white solid. LC/MS (m/z, M+H, Method 1): calc. 488, found 488.3; ¹H NMR (400 MHz, DMSO-d6, 30° C.) δ ppm 1.45-1.56 (m, 2H), 1.57-1.66 (m, 2H), 1.66-1.74 (m, 2H), 1.76-2.07 (m, 4H), 2.10-2.22 (m, 2H), 2.82-3.23 (m, 4H), 3.35-3.55 (m partially hidden, 1H), 3.55-3.79 (m, 1H), 4.56-4.77 (m, 1H), 7.06 (d, J=5 Hz, 1H), 7.45 (br d, J=9 Hz, 2H), 7.60 (d, J=9 Hz, 2H), 8.16 (br s, 1H), 11.85-12.75 (m, 1H)

### Example 10. 1-[3-[7-[1-[4-(Trifluoromethoxy)benzoyl]-4-piperidyl]-3H-imidazo[4,5-b]pyridin-2-yl]azetidin-1-yl]ethanone

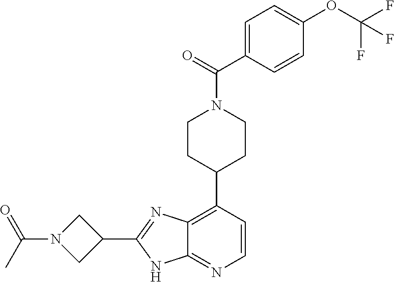 [SMILES: CC(=O)N1CC(c2nc3c(C4CCN(C(=O)c5ccc(OC(F)(F)F)cc5)CC4)ccnc3[nH]2)C1]

### Step 1: Tert-butyl 3-[7-[1-[4-(trifluoromethoxy)benzoyl]-4-piperidyl]-3H-imidazo[4,5-b]pyridin-2-yl]azetidine-1-carboxylate

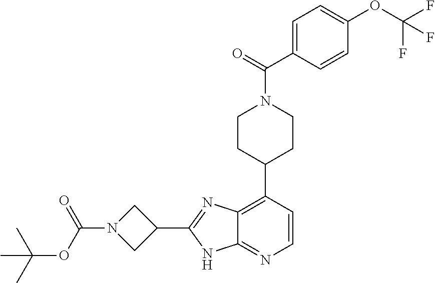 [SMILES: CC(C)(C)OC(=O)N1CC(c2nc3c(C4CCN(C(=O)c5ccc(OC(F)(F)F)cc5)CC4)ccnc3[nH]2)C1]

Step 1 of Example 10 was prepared following a similar procedure to that of step 1 of Example 5 from [4-(2,3-diamino-4-pyridyl)-1-piperidyl]-[4-(trifluoromethoxy)phenyl]methanone (150 mg, 0.39 mmol) and 1-tert-butoxycarbonylazetidine-3-carboxylic acid (79 mg, 0.39 mmol) to give 120 mg (56%) of tert-butyl 3-[7-[1-[4-(trifluoromethoxy)benzoyl]-4-piperidyl]-3H-imidazo[4,5-b]pyridin-2-yl]azetidine-1-carboxylate as a white solid. LC/MS (m/z, M+H): 546

### Step 2: 1-[3-[7-[1-[4-(Trifluoromethoxy)benzoyl]-4-piperidyl]-3H-imidazo[4,5-b]pyridin-2-yl]azetidin-1-yl]ethanone

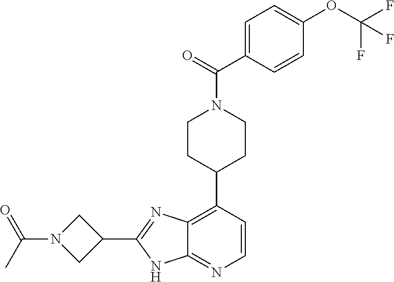 [SMILES: CC(=O)N1CC(c2nc3c(C4CCN(C(=O)c5ccc(OC(F)(F)F)cc5)CC4)ccnc3[nH]2)C1]

To a solution of tert-butyl 3-[7-[1-[4-(trifluoromethoxy)benzoyl]-4-piperidyl]-3H-imidazo[4,5-b]pyridin-2-yl]azetidine-1-carboxylate (116 mg, 0.21 mmol) in DCM (0.4 mL) at room temperature under argon atmosphere was added TFA (0.1 mL, 1.31 mmol) and the resulting mixture was stirred at room temperature for 6 hours. After 6 hours, additional 0.1 mL of TFA were added, and the resulting mixture was stirred for 12 additional hours at room temperature, then concentrated in vacuo, diluted with AcOEt and basified with a solution of NaOH 1N to pH>10, transferred in a separating funnel. The organic layer was separated and the aqueous layer was extracted three times with AcOEt. The combined organic layers were then washed with brine, dried over sodium sulfate, filtered and concentrated in vacuo. The resulting residue was dried under vacuum to give 95 mg (92%) of 1-[3-[7-[1-[4-(trifluoromethoxy)benzoyl]-4-piperidyl]-3H-imidazo[4,5-b]pyridin-2-yl]azetidin-1-yl]ethenone as a white solid. LC/MS (m/z, M+H, Method 2): calc. 488, found 488.2; ¹H NMR (400 MHz, DMSO-d6, 120° C.) δ ppm 1.79 (s, 3H), 1.85-2.05 (m, 4H), 3.02-3.23 (m, 2H), 3.36-3.58 (m, 1H), 4.00-4.12 (m, 1H), 4.17 (br d, J=11 Hz, 2H), 4.30-4.55 (m, 4H), 7.04 (d, J=5 Hz, 1H), 7.37 (br d, J=9 Hz, 2H), 7.56 (d, J=9 Hz, 2H), 8.10-8.30 (m, 1H), 11.70-12.80 (m, 1H)

### Example 11. [4-(2-Tetrahydropyran-4-yl-3H-imidazo[4,5-b]pyridin-7-yl)-1-piperidyl]-[4-(trifluoromethoxy)phenyl]methanone

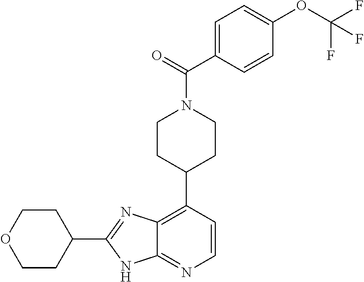 [SMILES: O=C(c1ccc(OC(F)(F)F)cc1)N1CCC(c2ccnc3[nH]c(C4CCOCC4)nc23)CC1]

### Step 1: Tert-butyl 4-(2-tetrahydropyran-4-yl-3H-imidazo[4,5-b]pyridin-7-yl)piperidine-1-carboxylate

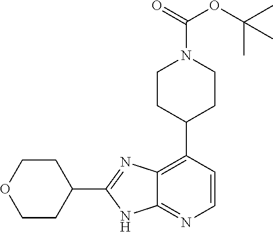 [SMILES: CC(C)(C)OC(=O)N1CCC(c2ccnc3[nH]c(C4CCOCC4)nc23)CC1]

To a solution of tetrahydropyran-4-carboxylic acid (67 mg, 0.51 mmol) in acetonitrile (5.1 mL) at room temperature under argon atmosphere was added CDI (100 mg, 0.62 mmol). The resulting mixture was then stirred one hour at room temperature. Then, tert-butyl 4-(2,3-diamino-4-pyridyl)piperidine-1-carboxylate (150 mg, 0.51 mmol) was added and the resulting mixture was stirred under reflux for 20 hours. The mixture was cooled down to room temperature and the formed precipitate was filtered off, washed with acetonitrile and dried under vacuum to give 142 mg (72%) of tert-butyl 4-(2-tetrahydropyran-4-yl-3H-imidazo[4,5-b]pyridin-7-yl)piperidine-1-carboxylate as a white solid. LC/MS (m/z, M+H): 387

### Step 2: 7-(4-Piperidyl)-2-tetrahydropyran-4-yl-3H-imidazo[4,5-b]pyridine, 2,2,2-trifluoroacetic acid

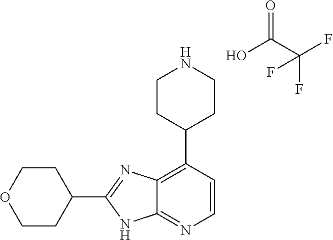

To a solution of tert-butyl 4-(2-tetrahydropyran-4-yl-3H-imidazo[4,5-b]pyridin-7-yl)piperidine-1-carboxylate (140 mg, 0.36 mmol) in DCM (1.7 mL) at room temperature under argon atmosphere was added TFA (0.4 mL, 5.19 mmol) and the resulting reaction mixture was stirred at room temperature for 12 hours. The reaction mixture was concentrated in vacuo to give 186 mg (100%) 7-(4-piperidyl)-2-tetrahydropyran-4-yl-3H-imidazo[4,5-b]pyridine, 2,2,2-trifluoroacetic acid as a yellow oil which was used in the next step without further purification. LC/MS (m/z, M+H): 287 (free base)

### Step 3: [4-(2-Tetrahydropyran-4-yl-3H-imidazo[4,5-b]pyridin-7-yl)-1-piperidyl]-[4-(trifluoromethoxy)phenyl]methanone

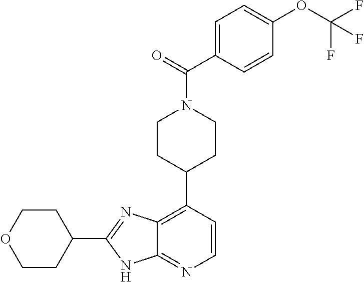 [SMILES: O=C(c1ccc(OC(F)(F)F)cc1)N1CCC(c2ccnc3[nH]c(C4CCOCC4)nc23)CC1]

To a solution of 7-(4-piperidyl)-2-tetrahydropyran-4-yl-3H-imidazo[4,5-b]pyridine, 2,2,2-trifluoroacetic acid (227 mg, 0.36 mmol) in DMF (4 mL) at room temperature under argon atmosphere was added DIEA (0.63 mL, 3.62 mmol) and five minutes later, HATU (131 mg, 0.34 mmol) portionwise. The reaction mixture was then stirred for 2 hours at room temperature. The reaction mixture was then diluted with 20 mL of water, extracted twice with AcOEt. The combined organic layers were washed with brine, dried over sodium sulfate, filtered and concentrated. The resulting residue was solubilized in a minimum of DCM, and pentane was progressively added until a white precipitate appeared, which was filtered, washed with pentane and dried under vacuum to give 158 mg of a grey solid. This later was diluted with AcOEt, washed with a 1 N solution of NaOH. The organic layer was separated, and the aqueous layer was extracted twice with AcOEt. The combined organic layers were then washed with brine, dried over sodium sulfate, filtered and concentrated in vacuo. The resulting residue was then triturated in Et₂O, filtered and dried under vacuum to give 105 mg (61%) of [4-(2-tetrahydropyran-4-yl-3H-imidazo[4,5-b]pyridin-7-yl)-1-piperidyl]-[4-(trifluoromethoxy)phenyl]methanone as a pale yellow solid. LC/MS (m/z, M+H, Method 2): calc. 475, found 475.2; ¹H NMR (400 MHz, DMSO-d6, 101° C.) δ ppm 1.80-2.02 (m, 8H), 3.05-3.24 (m, 3H), 3.34-3.46 (m, 1H), 3.44-3.54 (m, 2H), 3.95 (dt, J=11, 3 Hz, 2H), 4.05-4.30 (m, 2H), 7.02 (d, J=5 Hz, 1H), 7.38 (d, J=8 Hz, 2H), 7.56 (d, J=8 Hz, 2H), 8.15 (d, J=5 Hz, 1H), 11.80-12.80 (m, 1H)

### Example 12: [2-Amino-4-(trifluoromethoxy)phenyl]-[4-[2-(1-methyl-4-piperidyl)-3H-imidazo[4,5-b]pyridin-7-yl]-1-piperidyl]methanone

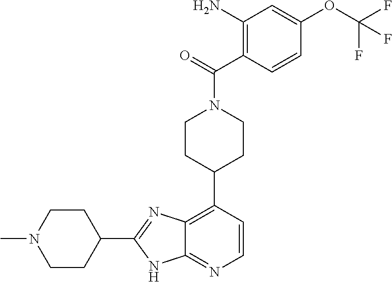 [SMILES: CN1CCC(c2nc3c(C4CCN(C(=O)c5ccc(OC(F)(F)F)cc5N)CC4)ccnc3[nH]2)CC1]

### Step 1: Tert-butyl 4-[2-(1-methyl-4-piperidyl)-3H-imidazo[4,5-b]pyridin-7-yl]piperidine-1-carboxylate

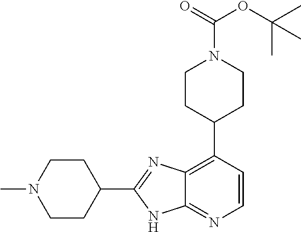 [SMILES: CN1CCC(c2nc3c(C4CCN(C(=O)OC(C)(C)C)CC4)ccnc3[nH]2)CC1]

Step 1 of Example 12 was prepared following a similar procedure to that of step 1 of Example 11 from tert-butyl 4-(2,3-diamino-4-pyridyl)piperidine-1-carboxylate (120 mg, 0.41 mmol) and 1-methylpiperidine-4-carboxylic acid (59 mg, 0.41 mmol) to give 94 mg (57%) of tert-butyl 4-[2-(1-methyl-4-piperidyl)-3H-imidazo[4,5-b]pyridin-7-yl]piperidine-1-carboxylate as a pale yellow solid. LC/MS (m/z, M+H): 400

### Step 2: 2-(1-Methyl-4-piperidyl)-7-(4-piperidyl)-3H-imidazo[4,5-b]pyridine, 2,2,2-trifluoroacetic acid

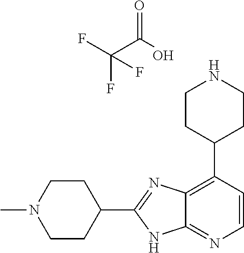

Step 2 of Example 12 was prepared following a similar procedure to that of step 2 of Example 11 from tert-butyl 4-[2-(1-methyl-4-piperidyl)-3H-imidazo[4,5-b]pyridin-7-yl]piperidine-1-carboxylate (93 mg, 0.23 mmol) and TFA (0.2 mL, 2.61 mmol) to give 149 mg (100%) of 2-(1-methyl-4-piperidyl)-7-(4-piperidyl)-3H-imidazo[4,5-b]pyridine, 2,2,2-trifluoroacetic acid as a yellow wax which was engaged in the next step without further purification. LC/MS (m/z, M+H): 300 (free base)

### Step 3: [2-Amino-4-(trifluoromethoxy)phenyl]-[4-[2-(1-methyl-4-piperidyl)-3H-imidazo[4,5-b]pyridin-7-yl]-1-piperidyl]methanone

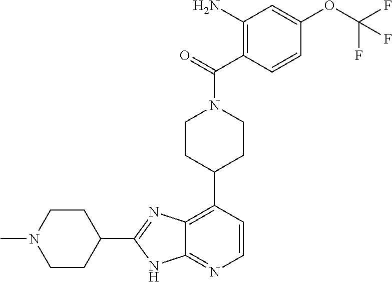 [SMILES: CN1CCC(c2nc3c(C4CCN(C(=O)c5ccc(OC(F)(F)F)cc5N)CC4)ccnc3[nH]2)CC1]

Step 3 of Example 12 was prepared following a similar procedure to that of step 3 of Example 11 from 2-(1-methyl-4-piperidyl)-7-(4-piperidyl)-3H-imidazo[4,5-b]pyridine, 2,2,2-trifluoroacetic acid (96 mg, 0.23 mmol) and 2-amino-4-(trifluoromethoxy)benzoic acid (52 mg, 0.2 mmol) to give 57 mg (49%) of [2-amino-4-(trifluoromethoxy)phenyl]-[4-[2-(1-methyl-4-piperidyl)-3H-imidazo[4,5-b]pyridin-7-yl]-1-piperidyl]methanone as a white solid. LC/MS (m/z, M+H, Method 1): calc. 503, found 503.2; ¹H NMR (400 MHz, DMSO-d6, 30° C., mixture of conformer 70/30) δ ppm 1.68-2.08 (m, 10H), 2.19 (s, 2.1H), 2.21 (br s, 0.9H), 2.73-2.93 (m, 3H), 2.98-3.18 (m, 2H), 3.46 (m, 1H), 3.55-5.00 (m, 2H), 5.58 (s, 1.4H), 5.61 (br s, 0.6H), 6.49 (br d, J=9 Hz, 1H), 6.66 (br s, 1H), 7.01-7.09 (m, 1H), 7.11-7.20 (m, 1H), 8.14 (d, J=5 Hz, 0.7H), 8.23 (d, J=5 Hz, 0.3H), 12.33 (s, 0.3H), 12.71 (br s, 0.7H)

### Example 13: [4-[2-(1-Methyl-4-piperidyl)-3H-imidazo[4,5-b]pyridin-7-yl]-1-piperidyl]-[4-(trifluoromethoxy)phenyl]methanone

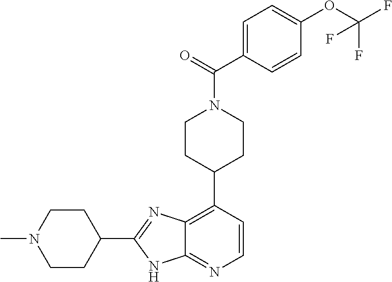 [SMILES: CN1CCC(c2nc3c(C4CCN(C(=O)c5ccc(OC(F)(F)F)cc5)CC4)ccnc3[nH]2)CC1]

Example 13 was prepared following a similar procedure to that of step 3 of Example 11 from 2-(1-methyl-4-piperidyl)-7-(4-piperidyl)-3H-imidazo[4,5-b]pyridine, 2,2,2-trifluoroacetic acid (231 mg, 0.44 mmol) and 4-(trifluoromethoxy)benzoic acid (135 mg, 0.66 mmol) to give 46 mg (21%) of [4-[2-(1-methyl-4-piperidyl)-3H-imidazo[4,5-b]pyridin-7-yl]-1-piperidyl]-[4-(trifluoromethoxy)phenyl]methanone as a pale yellow solid. LC/MS (m/z, M+H, Method 1): calc. 488, found 488.2; ¹H NMR (400 MHz, DMSO-d6, 30° C., mixture of conformer 70/30) δ ppm 1.70-2.12 (m, 10H), 2.22 (s, 2.1H), 2.24 (br s, 0.9H), 2.73-3.08 (m, 4H), 3.18-3.40 (m hidden, 1H), 3.43-3.56 (m, 1H), 3.59-3.79 (m, 1H), 4.52-4.81 (m, 1H), 7.05-7.11 (m, 1H), 7.46 (br d, J=9 Hz, 2H), 7.58-7.66 (m, 2H), 8.15 (d, J=5 Hz, 0.7H), 8.24 (d, J=5 Hz, 0.3H), 12.36 (s, 03H), 12.73 (s, 0.7H)

### Example 14: [4-(2-Methyl-3H-imidazo[4,5-b]pyridin-7-yl)-1-piperidyl]-[4-(trifluoromethoxy)phenyl]methanone

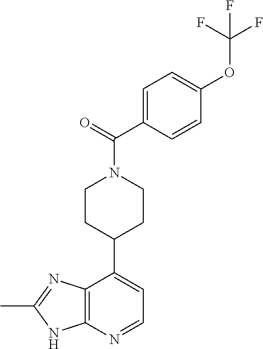 [SMILES: Cc1nc2c(C3CCN(C(=O)c4ccc(OC(F)(F)F)cc4)CC3)ccnc2[nH]1]

### Step 1: Tert-butyl 4-(2-methyl-3H-imidazo[4,5-b]pyridin-7-yl)piperidine-1-carboxylate

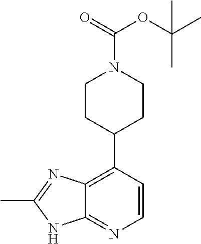 [SMILES: Cc1nc2c(C3CCN(C(=O)OC(C)(C)C)CC3)ccnc2[nH]1]

To tert-butyl 4-(2,3-diamino-4-pyridyl)piperidine-1-carboxylate (200 mg, 0.68 mmol) at room temperature under argon atmosphere was added 1,1,1-triethoxyethane (0.5 mL, 2.93 mmol) and ytterbium(III) trifluoromethanesulfonate (21 mg, 0.034 mmol). The resulting mixture was stirred at 90° C. for 1.5 hour. The reaction mixture was then cooled down to room temperature, solubilized in AcOEt, transferred in a separating funnel, washed with water and brine. The organic layer was separated and the aqueous phase was extracted with AcOEt. The combined organic layers were dried over sodium sulfate, filtered and concentrated in vacuo. The resulting residue was purified by flash chromatography (SiO₂ 12 g eluting with DCM 90/MeOH 10) to give 101 mg (46%) of tert-butyl 4-(2-methyl-3H-imidazo[4,5-b]pyridin-7-yl)piperidine-1-carboxylate as a pale brown solid. LC/MS (m/z, M+H): 317

### Step 2: 2-Methyl-7-(4-piperidyl)-3H-imidazo[4,5-b]pyridine, 2,2,2-trifluoroacetic acid

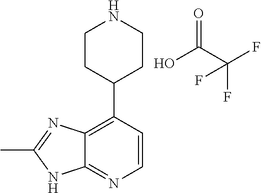

Step 2 of Example 14 was prepared following a similar procedure to that of step 2 of Example 11 from 4-(2-methyl-3H-imidazo[4,5-b]pyridin-7-yl)piperidine-1-carboxylate (95 mg, 0.30 mmol) and TFA (0.15 mL, 1.95 mmol) to give 100 mg (100%) of 2-(1-methyl-4-piperidyl)-7-(4-piperidyl)-3H-imidazo[4,5-b]pyridine, 2,2,2-trifluoroacetic acid as a yellow wax which was engaged in the next step without further purification. LC/MS (m/z, M+H): 217 (free base)

### Step 3: [4-(2-Methyl-3H-imidazo[4,5-b]pyridin-7-yl)-1-piperidyl]-[4-(trifluoromethoxy)phenyl]methanone

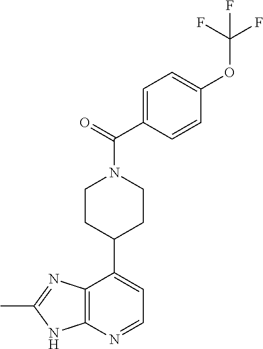 [SMILES: Cc1nc2c(C3CCN(C(=O)c4ccc(OC(F)(F)F)cc4)CC3)ccnc2[nH]1]

To a solution of 2-(1-methyl-4-piperidyl)-7-(4-piperidyl)-3H-imidazo[4,5-b]pyridine, 2,2,2-trifluoroacetic acid (100 mg, 0.3 mmol) in DMF (2.8 mL) at room temperature under argon atmosphere were successively added DIEA (0.2 mL, 1 mmol), 4-(trifluoromethoxy)benzoic acid (60 mg, 0.3 mmol), EDC (70 mg, 0.4 mmol) and 4-DMAP (7 mg, 0.06 mmol) and the resulting mixture was stirred at room temperature for 3 hours. The reaction mixture was then diluted with 20 mL of water, transferred in a separating funnel and extracted twice with AcOEt. The combined organic layers were washed with brine, dried over sodium sulfate, filtered and concentrated in vacuo. The resulting residue was purified by flash chromatography (SiO₂ 12 g, eluting with DCM 90/MeOH 10). The obtained desired compound was then solubilized in a minimum of DCM, and pentane was progressively added until a white precipitate appeared, which was filtered off, washed with pentane and dried under vacuum to give 29 mg (20%) of [4-(2-methyl-3H-imidazo[4,5-b]pyridin-7-yl)-1-piperidyl]-[4-(trifluoromethoxy)phenyl]methanone as a white solid. LC/MS (m/z, M+H, Method 1): calc. 405, found 405.2; ¹H NMR (400 MHz, DMSO-d6, 30° C., mixture of conformer 70/30) δ ppm 1.63-2.06 (m, 4H), 2.43-2.59 (m partially hidden, 3H), 2.82-3.04 (m, 1H), 3.11-3.24 (m partially hidden, 1H), 3.40-3.51 (m, 1H), 3.59-3.80 (m, 1H), 4.53-4.81 (m, 1H), 7.02-7.09 (m, 1H), 7.45 (br d, J=9 Hz, 2H), 7.56-7.65 (m, 2H), 8.12 (d, J=5 Hz, 0.7H), 8.21 (d, J=5 Hz, 0.3H), 12.48 (s, 0.3H), 12.69 (br s, 0.7H)

### Example 15: [4-[2-(3-Methoxy-1-bicyclo[1.1.1]pentanyl)-3H-imidazo[4,5-b]pyridin-7-yl]-1-piperidyl]-[4-(trifluoromethoxy)phenyl]methanone

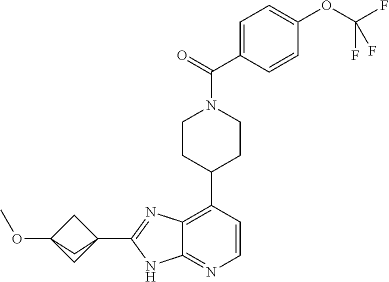 [SMILES: COC12CC(c3nc4c(C5CCN(C(=O)c6ccc(OC(F)(F)F)cc6)CC5)ccnc4[nH]3)(C1)C2]

### Step 1: [4-[2-(3-Methoxy-1-bicyclo[1.1.1]pentanyl)-3H-imidazo[4,5-b]pyridin-7-yl]-1-piperidyl]-[4-(trifluoromethoxy)phenyl]methanone

To a solution of 3-methoxybicyclo[1.1.1]pentane-1-carboxylic acid (54 mg, 0.38 mmol) in DMF (2 mL) at room temperature was added CDI (72 mg, 0.44 mmol) and the reaction mixture was stirred at 45° C. for 30 minutes. Then [4-(2,3-diamino-4-pyridyl)-1-piperidyl]-[4-(trifluoromethoxy)phenyl]methanone (120 mg, 0.32 mmol) was added and the resulting reaction mixture was stirred at 80° C. for 46 hours. Then, a mixture of 3-methoxybicyclo[1.1.1]pentane-1-carboxylic acid (18 mg, 0.12 mmol), CDI (24 mg, 0.14 mmol) in DMF (0.5 mL) (prepared aside by stirring for 30 minutes at room temperature) was added to the reaction mixture and the resulting mixture was stirred for 6 additional hours at 80° C. The reaction mixture was cooled down to room temperature, AcOEt and a saturated aqueous solution of NaHCO₃ were added, and the whole mixture was transferred to a separating funnel. The organic layer was separated, washed with water, dried over sodium sulfate, filtered and concentrated to dryness. The resulting residue was then purified by flash chromatography (SiO₂ 12 g eluting with DCM 90/MeOH 10) to give 136 mg (89%) of [4-[2-(3-methoxy-1-bicyclo[1.1.1]pentanyl)-3H-imidazo[4,5-b]pyridin-7-yl]-1-piperidyl]-[4-(trifluoromethoxy)phenyl]methanone as an orange solid. LC/MS (m/z, M+H, Method 1): calc. 487, found 487.2; ¹H NMR (400 MHz, DMSO-d6, 30° C., mixture of conformer 80/20) δ ppm 1.67-2.06 (m, 4H), 2.31 (s, 4.8H), 2.33 (s, 1.2H), 2.87-3.09 (m, 1H), 3.28 (s, 2.4H), 3.27-3.44 (m partially hidden, 1H), 3.30 (s, 0.6H), 3.47-3.59 (m, 1H), 3.59-3.90 (m, 1H), 4.48-4.84 (m, 1H), 7.12 (d, J=5 Hz, 1H), 7.46 (d, J=8 Hz, 2H), 7.56-7.68 (m, 2H), 8.19 (d, J=5 Hz, 0.8H), 8.27 (d, J=5 Hz, 0.2H), 12.50 (s, 0.2H), 12.99 (s, 0.8H)

### Example 16: 5-[7-[1-[4-(Trifluoromethoxy)benzoyl]-4-piperidyl]-3H-imidazo[4,5-b]pyridin-2-yl]piperidin-2-one

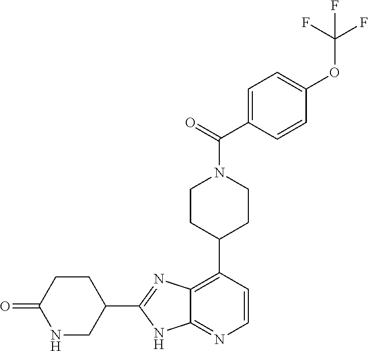 [SMILES: O=C1CCC(c2nc3c(C4CCN(C(=O)c5ccc(OC(F)(F)F)cc5)CC4)ccnc3[nH]2)CN1]

Example 16 was prepared following a similar procedure to that of step 1 of Example 5 from [4-(2,3-diamino-4-pyridyl)-1-piperidyl]-[4-(trifluoromethoxy)phenyl]methanone (252 mg, 0.66 mmol) and 6-oxopiperidine-3-carboxylic acid (100 mg, 0.70 mmol) with CDI (119 mg, 0.73 mmol) in acetonitrile (5 mL) for 16 hours under reflux to give 165 mg (48%) of 5-[7-[1-[4-(trifluoromethoxy)benzoyl]-4-piperidyl]-3H-imidazo[4,5-b]pyridin-2-yl]piperidin-2-one as a white solid. LC/MS (m/z, M+H, Method 2): calc. 488, found 488.2; ¹H NMR (400 MHz, DMSO-d6, 101° C.) δ ppm 1.81-2.01 (m, 4H), 2.07-2.29 (m, 2H), 2.29-2.43 (m, 2H), 3.06-3.19 (m, 2H), 3.28-3.49 (m, 2H), 3.55-3.63 (m, 2H), 4.05-4.25 (m, 2H), 7.04 (d, J=5 Hz, 1H), 7.17 (br s, 1H), 7.38 (d, J=8 Hz, 2H), 7.56 (d, J=8 Hz, 2H), 8.17 (d, J=5 Hz, 1H), 12.0-12.85 (m, 1H)

### Example 17: [4-(Trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone

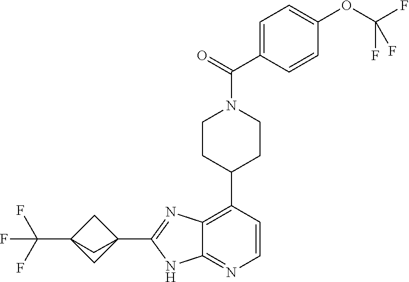 [SMILES: O=C(c1ccc(OC(F)(F)F)cc1)N1CCC(c2ccnc3[nH]c(C45CC(C(F)(F)F)(C4)C5)nc23)CC1]

Example 17 was prepared following a similar procedure to that of step 1 of Example 15 from [4-(2,3-diamino-4-pyridyl)-1-piperidyl]-[4-(trifluoromethoxy)phenyl]methanone (150 mg, 0.39 mmol) and 3-(trifluoromethyl)bicyclo[1.1.1]pentane-1-carboxylic acid (85 mg, 0.47 mmol) with CDI (89 mg, 0.55 mmol) in DMF (2 mL) for 20 hours at 80° C. to give 179 mg (86%) of [4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone as an orange solid. LC/MS (m/z, M+H, Method 1): calc. 525, found 525.3; ¹H NMR (400 MHz, DMSO-d6, 30° C.) δ ppm 1.65-2.03 (m, 4H), 2.46 (s, 6H), 2.85-3.09 (m, 1H), 3.15-3.41 (m hidden, 1H), 3.45-3.90 (m, 2H), 4.50-4.85 (m, 1H), 7.15 (d, J=5 Hz, 1H), 7.46 (d, J=8 Hz, 2H), 7.60 (d, J=8 Hz, 2H), 8.23 (br s, 1H), 12.4-13.5 (m, 1H)

### Example 18: [4-(3H-imidazo[4,5-b]pyridin-7-yl)-1-piperidyl]-[4-(trifluoromethoxy)phenyl]methanone

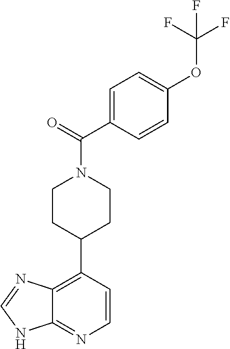 [SMILES: O=C(c1ccc(OC(F)(F)F)cc1)N1CCC(c2ccnc3[nH]cnc23)CC1]

To a solution of 4-amino-4-(trifluoromethyl)cyclohexane-1-carboxylic acid hydrochloride (40 mg, 0.16 mmol) in DMF (0.5 mL) and pyridine (0.5 mL) at room temperature was added CDI (31 mg, 0.18 mmol) and the reaction mixture was stirred at 45° C. for 30 minutes. Then [4-(2,3-diamino-4-pyridyl)-1-piperidyl]-[4-(trifluoromethoxy)phenyl]methanone (50 mg, 0.13 mmol) was added and the resulting reaction mixture was stirred at 80° C. for 16 hours. Then, the mixture was transferred in a microwave vial and pyridine (0.2 mL), DMF (0.4 mL) and CDI (15 mg, 0.09 mmol) were added. The vial was sealed and then irradiated under stirring for 15 minutes at 120° C., then 20 minutes at 150° C. CDI (20 mg, 0.12 mmol) was added to the mixture, the vial was sealed and irradiated under stirring for 1 hour at 170° C. Then, DIPEA (67 μL, 0.39 mmol) was added to the mixture then it was stirred at room temperature for 30 minutes and irradiated under microwave at 170° C. for 1 hour. After one hour, CDI (30 mg, 0.18 mmol) was added and the mixture was irradiated for 1 hour at 170° C. To the mixture were added 4-amino-4-(trifluoromethyl)cyclohexane-1-carboxylic acid hydrochloride (21 mg, 0.08 mmol), DIPEA (37 μL, 0.20 mmol), and after 10 minutes of stirring, CDI (18 mg, 0.11 mmol). The vial was then irradiated 4 hours at 170° C. CDI (30 mg, 0.18 mmol) was added and the vial was irradiated at 170° C. for 2 hours. Finally, CDI (30 mg, 0.18 mmol) was added and the vial was irradiated at 170° C. for 1 hour. The whole mixture was then transferred in a separating funnel with AcOEt (50 mL) and water (50 mL). The organic layer was separated, and then aqueous layer was extracted twice with AcOEt (2×20 mL). The organic layers were then gathered, dried over magnesium sulphate, filtered and concentrated under vacuum. The resulting residue was then purified by flash chromatography (SiO₂ 20 g eluting with DCM 90/MeOH 10/NH₄OH 1) to give 12 mg (23% yield) of [4-(3H-imidazo[4,5-b]pyridin-7-yl)-1-piperidyl]-[4-(trifluoromethoxy)phenyl]methanone as a solid. LC/MS (m/z, M+H, Method 2): calc. 391, found 391.2; ¹H NMR (500 MHz, DMSO-d6, 30° C.) δ ppm 1.76-2.09 (m, 4H), 2.84-3.05 (m, 1H), 3.20-3.40 (m hidden, 1H), 3.40-3.52 (m, 1H), 3.62-3.80 (m, 1H), 4.59-4.77 (m, 1H), 7.15 (d, J=5 Hz, 1H), 7.47 (d, J=8 Hz, 2H), 7.61 (d, J=8 Hz, 2H), 8.28 (d, J=5 Hz, 1H), 8.38 (s, 1H), 11.70-13.65 (m, 1H)

### Example 19: [4-[2-(Oxetan-3-yl)-3H-imidazo[4,5-b]pyridin-7-yl]-1-piperidyl]-[4-(trifluoromethoxy)phenyl]methanone

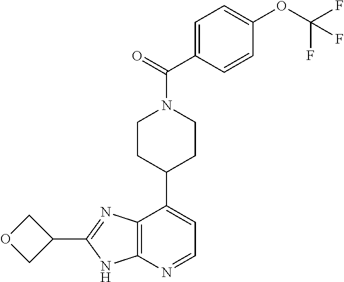 [SMILES: O=C(c1ccc(OC(F)(F)F)cc1)N1CCC(c2ccnc3[nH]c(C4COC4)nc23)CC1]

To a solution of oxetane-3-carboxylic acid (57 mg, 0.44 mmol) in DMF (1.2 mL) was added CDI (83 mg, 0.51 mmol) and the resulting mixture was stirred at room temperature for 1 hour. Then, [4-(2,3-diamino-4-pyridyl)-1-piperidyl]-[4-(trifluoromethoxy)phenyl]methanone (135 mg, 0.35 mmol) was added followed by 0.8 mL of pyridine. The resulting mixture was then stirred at 80° C. for 24 hours. Then, to the reaction mixture was added a pre-mixed solution containing oxetane-3-carboxylic acid (72 mg, 0.70 mmol), DMF (1 mL) and CDI (135 mg, 0.83 mmol) (mixed together at room temperature for one hour). The resulting reaction mixture was then stirred at 80° C. for 48 hours, then cooled down to room temperature, transferred in a separating funnel containing AcOEt and a saturated aqueous solution of NaHCO₃. The organic layer was then separated, washed with brine, dried over sodium sulfate, filtered and concentrated in vacuo. The resulting residue was then purified by flash chromatography (SiO₂ 25 g, eluting with DCM 93.5/MeOH 6.5/NH₄OH 0.65) to give 20 mg (13%) of [4-[2-(oxetan-3-yl)-3H-imidazo[4,5-b]pyridin-7-yl]-1-piperidyl]-[4-(trifluoromethoxy)phenyl]methanone as a pale brown solid. LC/MS (m/z, M+H, Method 1): calc. 447, found 447.2; ¹H NMR (400 MHz, DMSO-d6, 30° C.) δ ppm 1.65-2.11 (m, 4H), 2.85-3.09 (m, 1H), 3.15-3.55 (m partially hidden, 2H), 3.57-3.82 (m, 1H), 4.53 (m, 1H), 4.59-4.80 (m, 1H), 4.89-5.01 (m, 4H), 7.13 (d, J=5 Hz, 1H), 7.46 (d, J=8 Hz, 2H), 7.61 (d, J=8 Hz, 2H), 8.23 (br d, J=5 Hz, 1H), 12.3-13.6 (m, 1H)

### Example 20: (Rac)-[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone

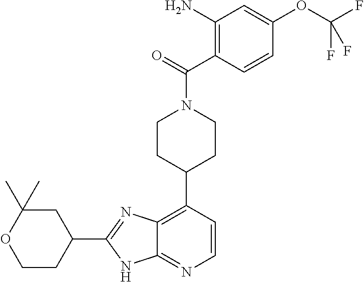 [SMILES: CC1(C)CC(c2nc3c(C4CCN(C(=O)c5ccc(OC(F)(F)F)cc5N)CC4)ccnc3[nH]2)CCO1]

### Step 1: (Rac)-tert-butyl 4-[2-(2,2-dimethyltetrahydropyran-4-yl)-3H-imidazo[4,5-b]pyridin-7-yl]piperidine-1-carboxylate

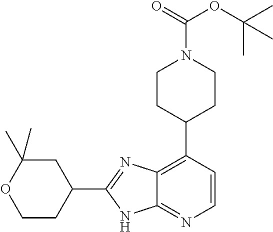 [SMILES: CC(C)(C)OC(=O)N1CCC(c2ccnc3[nH]c(C4CCOC(C)(C)C4)nc23)CC1]

To a solution of (rac)-2,2-dimethyltetrahydropyran-4-carboxylic acid (162 mg, 1.03 mmol) in a mixture of DMF/pyridine (1.5 mL/1.5 mL) was added CDI (194 mg, 1.20 mmol). The resulting mixture was stirred at 45° C. for 15 minutes. Then, tert-butyl 4-(2,3-diamino-4-pyridyl)piperidine-1-carboxylate (250 mg, 0.86 mmol) was added and the reaction mixture was stirred at 80° C. for 12 hours. The reaction mixture was concentrated to dryness, diluted with AcOEt, washed with a saturated aqueous solution of NaHCO₃ and with brine. The organic layer was then dried over Na₂SO₄, filtered and concentrated. The resulting residue was purified by flash chromatography (SiO₂ 15 g, eluting with DCM 95/MeOH 5/NH₄OH 0.5). The pure fractions were collected, concentrated to give 273 mg (77%) of (rac)-tert-butyl 4-[2-(2,2-dimethyltetrahydropyran-4-yl)-3H-imidazo[4,5-b]pyridin-7-yl]piperidine-1-carboxylate as an orange powder. LC/MS (m/z, M+H): 415

### Step 2: (Rac)-2-(2,2-dimethyltetrahydropyran-4-yl)-7-(4-piperidyl)-3H-imidazo[4,5-b]pyridine, dihydrochloride

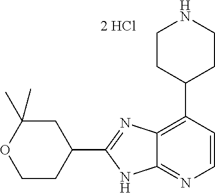 [SMILES: CC1(C)CC(c2nc3c(C4CCNCC4)ccnc3[nH]2)CCO1]

Step 2 of Example 20 was prepared following a similar procedure to that of step 4 of Examples 1 and 2 from (rac)-tert-butyl 4-[2-(2,2-dimethyltetrahydropyran-4-yl)-3H-imidazo[4,5-b]pyridin-7-yl]piperidine-1-carboxylate (270 mg, 0.65 mmol) using HCl 4N solution in 1,4-dioxane (2.44 mL, 9.77 mmol) in DCM/MeOH (3 mL/3 mL) to give 252 mg (100%) of (rac)-2-(2,2-dimethyltetrahydropyran-4-yl)-7-(4-piperidyl)-3H-imidazo[4,5-b]pyridine; dihydrochloride as a pale brown solid. LC/MS (m/z, M+H): 315 (free base)

### Step 3: (Rac)-[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone

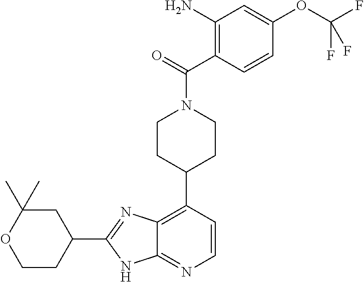 [SMILES: CC1(C)CC(c2nc3c(C4CCN(C(=O)c5ccc(OC(F)(F)F)cc5N)CC4)ccnc3[nH]2)CCO1]

Step 3 of Example 20 was prepared following a similar procedure to that of step 5 of Examples 1 and 2 from (rac)-2-(2,2-dimethyltetrahydropyran-4-yl)-7-(4-piperidyl)-3H-imidazo[4,5-b]pyridine dihydrochloride (252 mg, 0.65 mmol) with 2-amino-4-(trifluoromethoxy)benzoic acid (151 mg, 0.68 mmol), HATU (272 mg, 0.71 mmol), DIPEA (0.57 mL, 3.25 mmol) in DMF (4.3 mL) to give 237 mg (70%) of (rac)-[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone as a pale brown solid. LC/MS (m/z, M+H, Method 1): calc. 518, found 518.3; ¹H NMR (400 MHz, DMSO-d6, 30° C., mixture of conformer 70/30) δ ppm 1.19 (s, 3H), 1.27 (s, 3H), 1.55-1.98 (m, 8H), 2.9 (−3.19 (m, 2H), 3.19-3.36 (m partially hidden, 1H), 3.40-3.56 (m, 1H), 3.65-3.80 (m, 2H), 3.83-4.70 (m, 1H), 5.58 (br s, 2H), 6.49 (dd, J=8, 1 Hz, 1H), 6.67 (d, J=1 Hz, 1H), 7.06 (d, J=5 Hz, 1H), 7.16 (d, J=8 Hz, 1H), 8.12-8.21 (m, 0.7H), 8.19-8.28 (m, 0.3H), 12.35 (br s, 0.3H), 12.72 (br s, 0.7H)

### Example 21: [4-[2-(Azetidin-3-yl)-3H-imidazo[4,5-b]pyridin-7-yl]-1-piperidyl]-[4-(trifluoromethoxy)phenyl]methanone

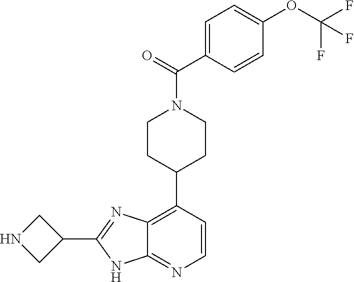 [SMILES: O=C(c1ccc(OC(F)(F)F)cc1)N1CCC(c2ccnc3[nH]c(C4CNC4)nc23)CC1]

### Step 1: Tert-butyl 3-[7-[1-[4-(trifluoromethoxy)benzoyl]-4-piperidyl]-3H-imidazo[4,5-b]pyridin-2-yl]azetidine-1-carboxylate

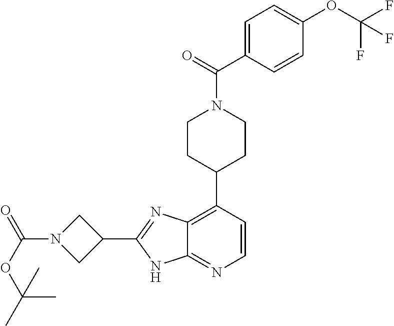 [SMILES: CC(C)(C)OC(=O)N1CC(c2nc3c(C4CCN(C(=O)c5ccc(OC(F)(F)F)cc5)CC4)ccnc3[nH]2)C1]

To a solution of 1-BOC-azetine-3-carboxylic acid (130 mg, 0.65 mmol) in pyridine (1 mL) and DMF (1 mL) was added CDI (105 mg, 0.65 mmol), and the resulting mixture was stirred for one hour at 40° C. Then, [4-(2,3-diamino-4-pyridyl)-1-piperidyl]-[4-(trifluoromethoxy)phenyl]methanone (200 mg, 0.52 mmol) was added and the reaction mixture was stirred at 80° C. for 12 hours. After 12 hours, the reaction mixture was cooled down to room temperature, concentrated to dryness and the resulting residue was purified by flash chromatography (SiO₂ 12 g, eluting with DCM/MeOH linear gradient) to give 295 mg (98%) tert-butyl 3-[7-[1-[4-(trifluoromethoxy)benzoyl]-4-piperidyl]-3H-imidazo[4,5-b]pyridin-2-yl]azetidine-1-carboxylate as an orange solid which was engaged directly in the next step.

### Step 2: [4-[2-(Azetidin-3-yl)-3H-imidazo[4,5-b]pyridin-7-yl]-1-piperidyl]-[4-(trifluoromethoxy)phenyl]methanone

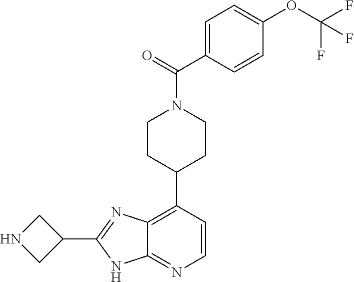 [SMILES: O=C(c1ccc(OC(F)(F)F)cc1)N1CCC(c2ccnc3[nH]c(C4CNC4)nc23)CC1]

To a solution of tert-butyl 3-[7-[1-[4-(trifluoromethoxy)benzoyl]-4-piperidyl]-3H-imidazo[4,5-b]pyridin-2-yl]azetidine-1-carboxylate (295 mg, 0.5407 mmol) in MeOH (6 mL) at 5° C. was dropwise added a 4 N solution of HCl in 1,4-dioxane (1.5 mL, 6.0 mmol). The resulting mixture was then stirred for 12 hours at room temperature. The resulting mixture was then concentrated to dryness. The resulting residue was then purified by preparative LC/MS (reverse phase, preparative LC/MS WATERS, Column C-18 SunFire (5 μm-30×100 mm) eluting with an acetonitrile gradient (+0.10% of formic acid) in water (+0.1% of formic acid)). Pure fractions were collected, concentrated and the resulting aqueous layer was basified to pH=9 with solid Na₂CO₃. DCM was added and the whole mixture was filtered through an hydrophobic cartridge (70 mL). The resulting organic filtrate was concentrated under reduce pressure to give 23 mg (9%) of [4-[2-(azetidin-3-yl)-3H-imidazo[4,5-b]pyridin-7-yl]-1-piperidyl]-[4-(trifluoromethoxy)phenyl]methanone as a pale yellow solid. LC/MS (m/z, M+H, Method 1): calc. 446, found 446.2; ¹H NMR (400 MHz, DMSO-d6, 30° C.) δ ppm 1.67-2.05 (m, 4H), 2.81-3.06 (m, 1H), 3.22-3.70 (m partially hidden, 3H), 3.74 (t, J=8 Hz, 2H), 3.95 (t, J=7 Hz, 2H), 4.03-4.16 (m, 1H), 4.56-4.85 (m, 1H), 7.10 (d, J=5 Hz, 1H), 7.46 (d, J=8 Hz, 2H), 7.61 (d, J=8 Hz, 2H), 8.20 (d, J=5 Hz, 1H)

### Examples 22 and 23: [4-(2-Morpholin-2-yl-3H-imidazo[4,5-b]pyridin-7-yl)-1-piperidyl]-[4-(trifluoromethoxy)phenyl]methanone, Isomers 1 and 2

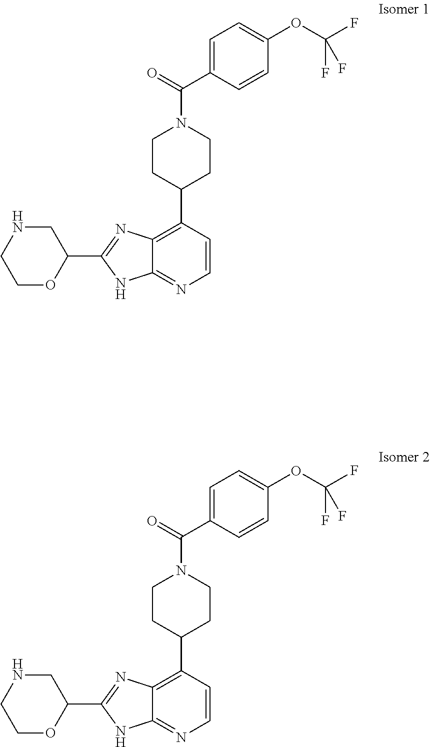 [SMILES: O=C(c1ccc(OC(F)(F)F)cc1)N1CCC(c2ccnc3[nH]c(C4CNCCO4)nc23)CC1 ; O=C(c1ccc(OC(F)(F)F)cc1)N1CCC(c2ccnc3[nH]c(C4CNCCO4)nc23)CC1]

### Step 1: (Rac)-tert-butyl 2-[7-[1-[4-(trifluoromethoxy)benzoyl]-4-piperidyl]-3H-imidazo[4,5-b]pyridin-2-yl]morpholine-4-carboxylate

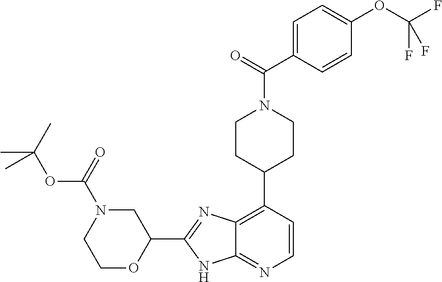 [SMILES: CC(C)(C)OC(=O)N1CCOC(c2nc3c(C4CCN(C(=O)c5ccc(OC(F)(F)F)cc5)CC4)ccnc3[nH]2)C1]

Step 1 of Examples 22 and 23 was prepared following a similar procedure to that of step 1 of Example 20 from (rac)-4-tert-butoxycarbonylmorpholine-2-carboxylic acid (109 mg, 0.47 mmol) with [4-(2,3-diamino-4-pyridyl)-1-piperidyl]-[4-(trifluoromethoxy)phenyl]methanone (150 mg, 0.39 mmol), CDI (89 mg, 0.55 mmol), in DMF/pyridine (1.5 mL/1.5 mL) to give 194 mg (85%) of (rac)-tert-butyl 2-[7-[1-[4-(trifluoromethoxy)benzoyl]-4-piperidyl]-3H-imidazo[4,5-b]pyridin-2-yl]morpholine-4-carboxylate as a pale brown solid. LC/MS (m/z, M+H): 576

### Step 2: (Rac)-[4-(2-morpholin-2-yl-3H-imidazo[4,5-b]pyridin-7-yl)-1-piperidyl]-[4-(trifluoromethoxy)phenyl]methanone

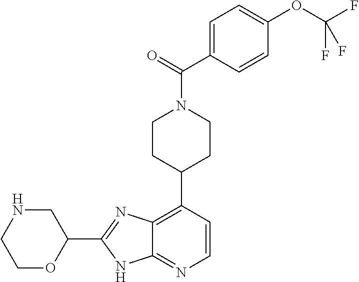 [SMILES: O=C(c1ccc(OC(F)(F)F)cc1)N1CCC(c2ccnc3[nH]c(C4CNCCO4)nc23)CC1]

Step 2 of Examples 22 and 23 was prepared following a similar procedure to that of step 2 of Example 21 from (rac)-tert-butyl 2-[7-[1-[4-(trifluoromethoxy)benzoyl]-4-piperidyl]-3H-imidazo[4,5-b]pyridin-2-yl]morpholine-4-carboxylate (190 mg, 0.33 mmol) using HCl 4N solution in 1,4-dioxane (1.24 mL, 4.95 mmol) in MeOH (4 mL) to give 143 mg (91%) of (rac)-[4-(2-morpholin-2-yl-3H-imidazo[4,5-b]pyridin-7-yl)-1-piperidyl]-[4-(trifluoromethoxy)phenyl]methanone as a pale brown solid. LC/MS (m/z, M+H): 476

Chiral separation of (rac)-[4-(2-morpholin-2-yl-3H-imidazo[4,5-b]pyridin-7-yl)-1-piperidyl]-[4-(trifluoromethoxy)phenyl]methanone (118 mg, 0.25 mmol), using a cellulose column (4-20 μm, 350×76.5 mm), eluting with Heptane 35% EtOH 65%+0.1% TEA followed by 100% of EtOH at 9 minutes of elution (flow rate 40 mL/min, UV detection at 265 nm) gave 47 mg (40%) of the first eluting Isomer 1 (Example 22) and 54 mg (46%) of Isomer 2 (Example 23) as white solids.

Isomer 1 (Example 22): LC/MS (m/z, M+H, Method 2): calc. 476, found 476.2; ¹H NMR (400 MHz, DMSO-d6, 30° C.) δ ppm 1.70-2.07 (m, 4H), 2.74-2.81 (m, 2H), 2.80-3.10 (m, 2H), 3.13 (br d, J=12 Hz, 1H), 3.20-3.57 (m partially hidden, 2H), 3.60-3.81 (m, 2H), 3.90 (br d, J=11 Hz, 1H), 4.55-4.84 (m, 2H), 7.12 (d, J=5 Hz, 1H), 7.46 (d, J=8 Hz, 2H), 7.61 (d, J=8 Hz, 2H), 8.24 (d, J=5 Hz, 1H), 12.39-13.49 (m, 1H)

Isomer 2 (Example 23): LC/MS (m/z, M+H, Method 2): calc. 476, found 476.2; ¹H NMR (400 MHz, DMSO-d6, 30° C.) δ ppm 1.70-2.07 (m, 4H), 2.74-2.81 (m, 2H), 2.80-3.10 (m, 2H), 3.13 (br d, J=12 Hz, 1H), 3.20-3.57 (m partially hidden, 2H), 3.60-3.81 (m, 2H), 3.90 (br d, J=11 Hz, 1H), 4.55-4.84 (m, 2H), 7.12 (d, J=5 Hz, 1H), 7.46 (d, J=8 Hz, 2H), 7.61 (d, J=8 Hz, 2H), 8.24 (d, J=5 Hz, 1H), 12.39-13.49 (m, 1H)

### Examples 24 and 25: [4-[2-(1-Methylpyrrolidin-3-yl)-3H-imidazo[4,5-b]pyridin-7-yl]-1-piperidyl]-[4-(trifluoromethoxy)phenyl]methanone, Isomers 1 and 2

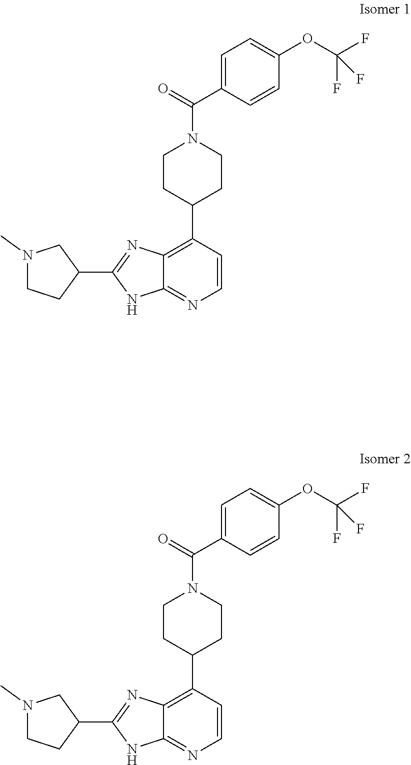 [SMILES: CN1CCC(c2nc3c(C4CCN(C(=O)c5ccc(OC(F)(F)F)cc5)CC4)ccnc3[nH]2)C1 ; CN1CCC(c2nc3c(C4CCN(C(=O)c5ccc(OC(F)(F)F)cc5)CC4)ccnc3[nH]2)C1]

### Step 1: (Rac)-tert-butyl 4-[2-(1-methylpyrrolidin-3-yl)-3H-imidazo[4,5-b]pyridin-7-yl]piperidine-1-carboxylate

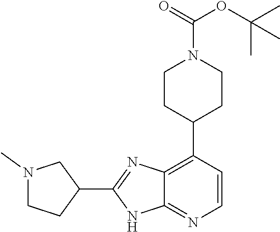 [SMILES: CN1CCC(c2nc3c(C4CCN(C(=O)OC(C)(C)C)CC4)ccnc3[nH]2)C1]

Step 1 of Examples 24 and 25 was prepared following a similar procedure to that of step 1 of Example 20 from tert-butyl 4-(2,3-diamino-4-pyridyl)piperidine-1-carboxylate (250 mg, 0.85 mmol) and 1-methylpyrrolidine-3-carboxylic acid (132 mg, 1.03 mmol) with CDI (194 mg, 1.20 mmol) in DMF/pyridine (1.5 mL/1.5 mL) at 80° C. for 12 hours to give 232 mg (70%) of (rac)-tert-butyl 4-[2-(1-methylpyrrolidin-3-yl)-3H-imidazo[4,5-b]pyridin-7-yl]piperidine-1-carboxylate as a pale brown solid. LC/MS (m/z, M+H): 386

### Step 2: (Rac)-2-(1-methylpyrrolidin-3-yl)-7-(4-piperidyl)-3H-imidazo[4,5-b]pyridine, hydrochloride

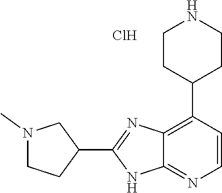 [SMILES: CN1CCC(c2nc3c(C4CCNCC4)ccnc3[nH]2)C1 ; Cl]

Step 2 of Examples 24 and 25 was prepared following a similar procedure to that of step 4 of Examples 1 and 2 from (rac)-tert-butyl 4-[2-(1-methylpyrrolidin-3-yl)-3H-imidazo[4,5-b]pyridin-7-yl]piperidine-1-carboxylate (228 mg, 0.59 mmol) using HCl 4N solution in 1,4-dioxane (1.48 mL, 5.91 mmol) in MeOH (4 mL) to give 233 mg (100%) of (rac)-2-(1-methylpyrrolidin-3-yl)-7-(4-piperidyl)-3H-imidazo[4,5-b]pyridine, hydrochloride as a pale brown wax. LC/MS (m/z, M+H): 286 (free base)

### Step 3: (Rac)-[4-[2-(1-methylpyrrolidin-3-yl)-3H-imidazo[4,5-b]pyridin-7-yl]-1-piperidyl]-[4-(trifluoromethoxy)phenyl]methanone

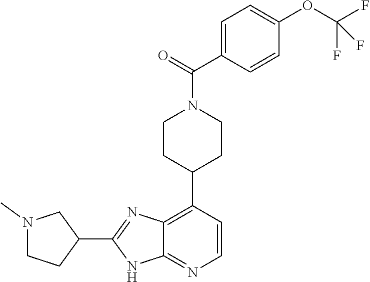 [SMILES: CN1CCC(c2nc3c(C4CCN(C(=O)c5ccc(OC(F)(F)F)cc5)CC4)ccnc3[nH]2)C1]

Step 3 of Example 24 and 25 was prepared following a similar procedure to that of step 5 of Examples 1 and 2 from (rac)-2-(1-methylpyrrolidin-3-yl)-7-(4-piperidyl)-3H-imidazo[4,5-b]pyridine, hydrochloride (233 mg, 0.59 mmol) and 4-(trifluoromethoxy)benzoic acid (134 mg, 0.65 mmol) with HATU (269 mg, 0.71 mmol) and DIPEA (0.51 mL, 2.95 mmol) in DMF (3.9 mL) to give 197 mg (70%) of (rac)-[4-[2-(1-methylpyrrolidin-3-yl)-3H-imidazo[4,5-b]pyridin-7-yl]-1-piperidyl]-[4-(trifluoromethoxy)phenyl]methanone as a pale brown solid. LC/MS (m/z, M+H): 474

Chiral separation of (rac)-[4-[2-(1-methylpyrrolidin-3-yl)-3H-imidazo[4,5-b]pyridin-7-yl]-1-piperidyl]-[4-(trifluoromethoxy)phenyl]methanone (160 mg, 0.34 mmol), using a chiralpak AY column (20 μm, 100×250 mm), eluting with (Heptane 80% EtOH 15% MeOH 5%)+0.1% TEA (flow rate 40 mL/min and UV detection at 280 nm) gave 69 mg (43%) of the first eluting isomer 1 (Example 24) and 72 mg (45%) of isomer 2 (Example 25) as white solids.

Isomer 1 (Example 24): LC/MS (m/z, M+H, Method 2): calc. 474, found 474.2; ¹H NMR (400 MHz, DMSO-d6, 30° C.) δ ppm 1.71-2.04 (m, 4H), 2.18-2.28 (m, 2H), 2.30 (s, 3H), 2.41-2.60 (m partially hidden, 1H), 2.60-2.75 (m, 2H), 2.80-3.08 (m, 2H), 3.26-3.51 (m partially hidden, 2H), 3.59 (quin, J=8 Hz, 1H), 3.55-3.80 (m, 1H), 4.58-4.79 (m, 1H), 7.08 (d, J=5 Hz, 1H), 7.46 (d, J=8 Hz, 2H), 7.60 (d, J=8 Hz, 2H), 8.17 (d, J=5 Hz, 1H), 12.1-13.2 (m, 1H)

Isomer 2 (Example 25): LC/MS (m/z, M+H, Method 2): calc. 474, found 474.2; ¹H NMR (400 MHz, DMSO-d6, 30° C.) δ ppm 1.71-2.04 (m, 4H), 2.18-2.28 (m, 2H), 2.30 (s, 3H), 2.41-2.60 (m partially hidden, 1H), 2.60-2.75 (m, 2H), 2.80-3.08 (m, 2H), 3.26-3.51 (m partially hidden, 2H), 3.59 (quin, J=8 Hz, 1H), 3.55-3.80 (m, 1H), 4.58-4.79 (m, 1H), 7.08 (d, J=5 Hz, 1H), 7.46 (d, J=8 Hz, 2H), 7.60 (d, J=8 Hz, 2H), 8.17 (d, J=5 Hz, 1H), 12.1-13.2 (m, 1H)

### Examples 26 and 27: [4-(2-Tetrahydrofuran-3-yl-3H-imidazo[4,5-b]pyridin-7-yl)-1-piperidyl]-[4-(trifluoromethoxy)phenyl]methanone, Isomers 1 and 2

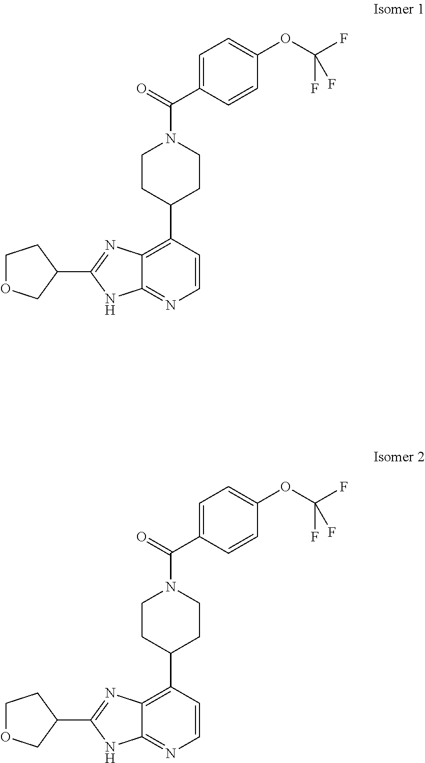 [SMILES: O=C(c1ccc(OC(F)(F)F)cc1)N1CCC(c2ccnc3[nH]c(C4CCOC4)nc23)CC1 ; O=C(c1ccc(OC(F)(F)F)cc1)N1CCC(c2ccnc3[nH]c(C4CCOC4)nc23)CC1]

### Step 1: (Rac)-[4-(2-tetrahydrofuran-3-yl-3H-imidazo[4,5-b]pyridin-7-yl)-1-piperidyl]-[4-(trifluoromethoxy)phenyl]methanone

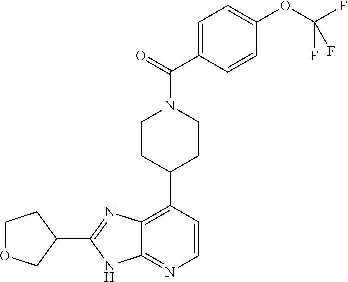 [SMILES: O=C(c1ccc(OC(F)(F)F)cc1)N1CCC(c2ccnc3[nH]c(C4CCOC4)nc23)CC1]

Step 1 of Examples 26 and 27 was prepared following a similar procedure to that of step 1 of Example 20 from (rac)-tetrahydrofuran-3-carboxylic acid (55 mg, 0.47 mmol) with [4-(2,3-diamino-4-pyridyl)-1-piperidyl]-[4-(trifluoromethoxy)phenyl]methanone (150 mg, 0.39 mmol), CDI (89 mg, 0.55 mmol), in DMF/pyridine (1.5 mL/1.5 mL) to give 143 mg (79%) of (rac)-[4-(2-tetrahydrofuran-3-yl-3H-imidazo[4,5-b]pyridin-7-yl)-1-piperidyl]-[4-(trifluoromethoxy)phenyl]methanone as a pale brown solid. LC/MS (m/z, M+H): 461

### Step 2: [4-(2-Tetrahydrofuran-3-yl-3H-imidazo[4,5-b]pyridin-7-yl)-1-piperidyl]-[4-(trifluoromethoxy)phenyl]methanone, Isomers 1 and 2

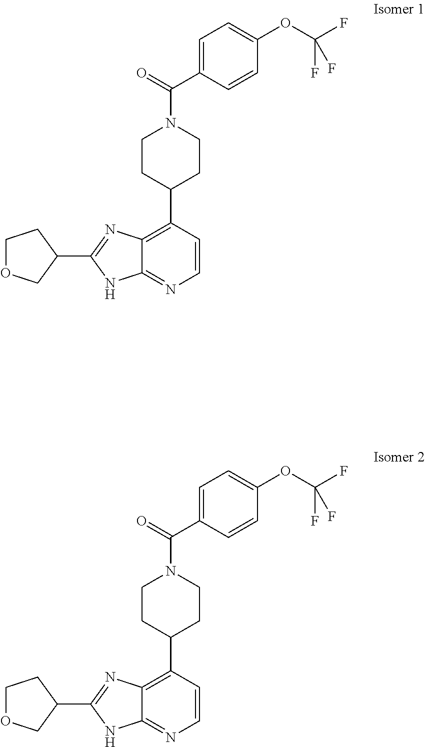 [SMILES: O=C(c1ccc(OC(F)(F)F)cc1)N1CCC(c2ccnc3[nH]c(C4CCOC4)nc23)CC1 ; O=C(c1ccc(OC(F)(F)F)cc1)N1CCC(c2ccnc3[nH]c(C4CCOC4)nc23)CC1]

Step 2 of Examples 26 and 27 consisted in a chiral separation of (rac)-[4-(2-tetrahydrofuran-3-yl-3H-imidazo[4,5-b]pyridin-7-yl)-1-piperidyl]-[4-(trifluoromethoxy)phenyl]methanone (110 mg, 0.24 mmol), using a chiralpak AY column (20 μm, 100×250 mm), eluting with (Heptane 70% EtOH 30%)+0.1% TEA (flow rate 40 mL/min, UV detection at 265 nm). Pure fractions of each enantiomer were collected separately and concentrated to furnish 44 mg (40%) of the first eluting Isomer 1 (Example 26) and 47 mg (43%) of Isomer 2 (Example 27) as white solids.

Isomer 1 (Example 26): LC/MS (m/z, M+H, Method 2): calc. 461, found 461.2; ¹H NMR (400 MHz, DMSO-d6, 30° C.) δ ppm 1.70-2.05 (m, 4H), 2.26-2.39 (m, 2H), 2.86-3.13 (m, 1H), 3.15-3.55 (m partially hidden, 2H), 3.57-3.75 (m, 2H), 3.82 (q, J=7 Hz, 1H), 3.89-3.97 (m, 2H), 4.06-4.15 (m, 1H), 4.52-4.80 (m, 1H), 7.09 (d, J=5 Hz, 1H), 7.45 (br d, J=9 Hz, 2H), 7.60 (d, J=9 Hz, 2H), 8.11-8.28 (m, 1H), 12.2-13.5 (m, 1H)

Isomer 2 (Example 27): LC/MS (m/z, M+H, Method 2): calc. 461, found 461.2; ¹H NMR (400 MHz, DMSO-d6, 30° C., mixture of conformer 70/30) δ ppm 1.69-2.05 (m, 4H), 2.26-2.39 (m, 2H), 2.82-3.06 (m, 1H), 3.11-3.34 (m hidden, 1H), 3.42-3.55 (m, 1H), 3.59-3.76 (m, 2H), 3.77-3.88 (m, 1H), 3.89-3.98 (m, 2H), 4.09 (br t, J=8 Hz, 1H), 4.52-4.88 (m, 2H), 7.09 (d, J=5 Hz, 1H), 7.45 (d, J=8 Hz, 2H), 7.60 (br d, J=9 Hz, 2H), 8.16 (d, J=5 Hz, 0.7H), 8.22-8.31 (m, 0.3H), 12.23-12.67 (m, 0.3H), 12.73-13.06 (m, 0.7H)

### Example 28: (Rac)-[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone

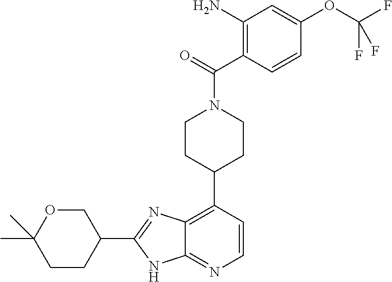 [SMILES: CC1(C)CCC(c2nc3c(C4CCN(C(=O)c5ccc(OC(F)(F)F)cc5N)CC4)ccnc3[nH]2)CO1]

### Step 1: (Rac)-tert-butyl 4-[2-(6,6-dimethyltetrahydropyran-3-yl)-3H-imidazo[4,5-b]pyridin-7-yl]piperidine-1-carboxylate

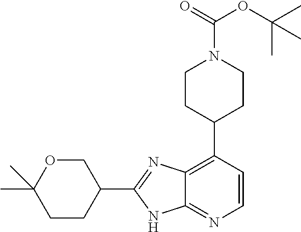 [SMILES: CC(C)(C)OC(=O)N1CCC(c2ccnc3[nH]c(C4CCC(C)(C)OC4)nc23)CC1]

Step 1 of Example 28 was prepared following a similar procedure to that of step 1 of Example 20 from (rac)-6,6-dimethyltetrahydropyran-3-carboxylic acid (162 mg, 1.03 mmol) with tert-butyl 4-(2,3-diamino-4-pyridyl)piperidine-1-carboxylate (250 mg, 0.85 mmol), CDI (194 mg, 1.20 mmol), in DMF/pyridine (1.5 mL/1.5 mL) to give 274 mg (77%) of (rac)-tert-butyl 4-[2-(6,6-dimethyltetrahydropyran-3-yl)-3H-imidazo[4,5-b]pyridin-7-yl]piperidine-1-carboxylate as a pale brown solid. LC/MS (m/z, M+H): 415

### Step 2: (Rac)-2-(6,6-dimethyltetrahydropyran-3-yl)-7-(4-piperidyl)-3H-imidazo[4,5-b]pyridine, dihydrochloride

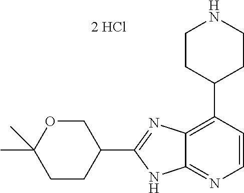 [SMILES: CC1(C)CCC(c2nc3c(C4CCNCC4)ccnc3[nH]2)CO1]

Step 2 of Example 28 was prepared following a similar procedure to that of step 4 of Examples 1 and 2 from (rac)-tert-butyl 4-[2-(6,6-dimethyltetrahydropyran-3-yl)-3H-imidazo[4,5-b]pyridin-7-yl]piperidine-1-carboxylate (271 mg, 0.65 mmol) using HCl 4N solution in 1,4-dioxane (2.44 mL, 9.77 mmol) in DCM/MeOH (3 mL/3 mL) to give 253 mg (100%) of (rac)-2-(6,6-dimethyltetrahydropyran-3-yl)-7-(4-piperidyl)-3H-imidazo[4,5-b]pyridine, dihydrochloride as a pale brown solid. LC/MS (m/z, M+H): 315 (free base)

### Step 3: (Rac)-[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone

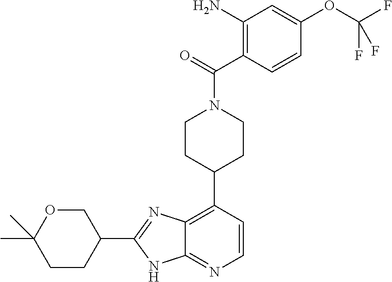 [SMILES: CC1(C)CCC(c2nc3c(C4CCN(C(=O)c5ccc(OC(F)(F)F)cc5N)CC4)ccnc3[nH]2)CO1]

Step 3 of Example 28 was prepared following a similar procedure to that of step 5 of Examples 1 and 2 from (rac)-2-(6,6-dimethyltetrahydropyran-3-yl)-7-(4-piperidyl)-3H-imidazo[4,5-b]pyridine, dihydrochloride (253 mg, 0.65 mmol) with 2-amino-4-(trifluoromethoxy)benzoic acid (152 mg, 0.68 mmol), HATU (273 mg, 0.72 mmol), DIPEA (0.57 mL, 3.25 mmol) in DMF (4.4 mL) to give 262 mg (77%) of (rac)-[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone as a pale brown solid. LC/MS (m/z, M+H, Method 1): calc. 518, found 518.3; ¹H NMR (400 MHz, DMSO-d6, 30° C., mixture of conformer 70/30) δ ppm 1.20 (s, 3H), 1.24 (s, 3H), 1.47-1.59 (m, 1H), 1.66 (br d, J=14 Hz, 1H), 1.70-1.94 (m, 4H), 1.94-2.19 (m, 2H), 2.87-3.22 (m, 3H), 3.38-3.55 (m, 1H), 3.64-4.84 (m, 4H), 5.59 (br s, 2H), 6.49 (br d, J=8 Hz, 1H), 6.67 (br d, J=1 Hz, 1H), 7.07 (d, J=5 Hz, 1H), 7.16 (d, J=8 Hz, 1H), 8.07-8.34 (m, 1H), 12.41 (br s, 0.3H), 12.82 (br s, 0.7H)

### Example 29: (Rac)-[2-amino-4-(trifluoromethoxy)phenyl]-[4-(2-morpholin-2-yl-3H-imidazo[4,5-b]pyridin-7-yl)-1-piperidyl]methanone

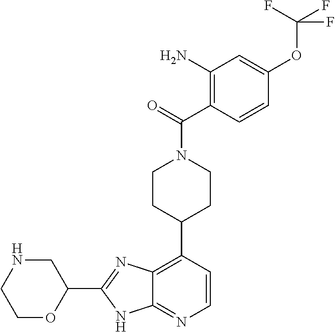 [SMILES: Nc1cc(OC(F)(F)F)ccc1C(=O)N1CCC(c2ccnc3[nH]c(C4CNCCO4)nc23)CC1]

### Step 1: 2-Nitro-4-(trifluoromethoxy)benzonitrile

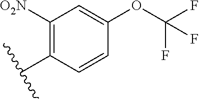 [SMILES: *c1ccc(OC(F)(F)F)cc1[N+](=O)[O-]]

To a solution of 2-bromo-4-(trifluoromethoxy)benzonitrile (90.0 g, 314 mmol) in DMF (540 mL) was added CuCN (31.0 g, 346 mmol, 75.6 mL) at 25° C. The mixture was heated to 150° C. and stirred at 150° C. for 1 hour. The reaction mixture was cooled to 30° C. and diluted with EtOAc (500 mL), filtered, and the filtrate was washed with brine (450 mL×3), dried over MgSO₄, filtered and concentrated to give 100 g of 2-nitro-4-trifluoromethoxybenzonitrile (100 g, crude) as a yellow oil.

### Step 2: 2-Nitro-4-(trifluoromethoxy)benzoic acid

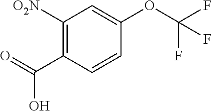 [SMILES: O=C(O)c1ccc(OC(F)(F)F)cc1[N+](=O)[O-]]

To a solution of H₂O (200 mL), H₂SO₄ (343 g, 3.50 mol, 186 mL) and AcOH (225 g, 3.75 mol, 214 mL) was added 2-nitro-4-trifluoromethoxybenzonitrile (100 g, 430 mmol) at 20° C. The solution was stirred at 120° C. for 16 hours. The reaction solution was then cooled to 25° C. The residue was poured into ice-water (600 mL) and stirred for 30 minutes. The solid was filtered and dissolved into EtOAc (400 mL). The organic phase was washed with brine (250 mL), dried over Na₂SO₄, filtered and concentrated under reduced pressure. The product was purified by silica gel column chromatography (THF/Petroleum ether=0-10%) to give 46 g (43%) of 2-nitro-4-(trifluoromethoxy)benzoic acid as a yellow solid.

### Step 3: 4-(4,4,5,5-Tetramethyl-1,3,2-dioxaborolan-2-yl)-1,2,3,6-tetrahydropyridine, hydrochloride

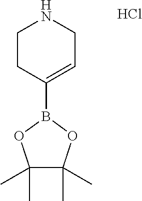

To a solution of tert-butyl 4-(4,4,5,5-tetramethyl-1,3,2-dioxaborolan-2-yl)-3,6-dihydro-2H-pyridine-1-carboxylate (75.0 g, 242 mmol) in EtOAc (75 mL) was added a 4N solution of HCl in EtOAc (4.00 M, 303 mL) at 10° C., then allowed to warm up to 25° C. The mixture was stirred at 25° C. for 4 hours. Then, the suspension was diluted with ethyl acetate (500 mL), filtered and the filter cake was concentrated under vacuum. The crude product was triturated with EtOAc (500 mL) at 25° C. for 30 minutes to give compound 45 g (75%) of 4-(4,4,5,5-tetramethyl-1,3,2-dioxaborolan-2-yl)-1,2,3,6-tetrahydropyridine, hydrochloride as a white solid.

### Step 4: [2-Nitro-4-(trifluoromethoxy)phenyl]-[4-(4,4,5,5-tetramethyl-1,3,2-dioxaborolan-2-yl)-3,6-dihydro-2H-pyridin-1-yl]methanone

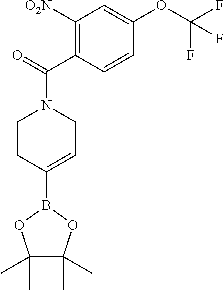 [SMILES: CC1(C)OB(C2=CCN(C(=O)c3ccc(OC(F)(F)F)cc3[N+](=O)[O-])CC2)OC1(C)C]

To a solution of 2-nitro-4-(trifluoromethoxy)benzoic acid (35.0 g, 139 mmol), di-isopropylethylamine (72.0 g, 557 mmol, 97.1 m) and HATU (63.5 g, 167 mmol) in DMF (245 mL) was added 4-(4,4,5,5-tetramethyl-1,3,2-dioxaborolan-2-yl)-1,2,3,6-tetrahydropyridine, hydrochloride (34.9 g, 142 mmol) at 20° C. The solution was stirred at 20° C. for 1.5 hours. To the reaction mixture was added a saturated aqueous solution of NaHCO₃ (750 mL) and AcOEt (1000 mL). The organic phase was washed with brine (500 mL×2) and then concentrated in vacuum to give a crude product. The crude was triturated with Petroleum ether (300 mL) at 15° C. for 4 hours to give 26.5 g (43%) of [2-nitro-4-(trifluoromethoxy)phenyl]-[4-(4,4,5,5-tetramethyl-1,3,2-dioxaborolan-2-yl)-3,6-dihydro-2H-pyridin-1-yl]methanone as a white solid.

### Step 5: [4-(2,3-Diamino-4-pyridyl)-3,6-dihydro-2H-pyridin-1-yl]-[2-nitro-4-(trifluoromethoxy)phenyl]methanone

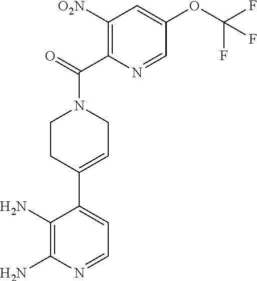 [SMILES: Nc1nccc(C2=CCN(C(=O)c3ncc(OC(F)(F)F)cc3[N+](=O)[O-])CC2)c1N]

In a 500 mL sealed tube, a solution of 4-chloropyridine-2,3-diamine (2.17 g, 15.1 mmol), [2-nitro-4-(trifluoromethoxy)phenyl]-[4-(4,4,5,5-tetramethyl-1,3,2-dioxaborolan-2-yl)-3,6-dihydro-2H-pyridin-1-yl]methanone (7.22 g, 16.3 mmol) and K₃PO₄ (9.62 g, 45.3 mmol) in 1,4-dioxane/water (180 mL/20 mL) was bubbled with argon for 5 minutes. Then, SPhos Pd G2 catalyst (1.42 g, 1.96 mmol) was added, the tube was sealed and stirred at 90° C. for 12 hours. Then, the reaction mixture was cooled down to room temperature, concentrated under vacuum to dryness. The resulting residue was diluted with AcOEt and a 1N aqueous solution of NaOH, transferred in a separating funnel. The organic layer was separated, washed with brine, twice with a 1N solution of HCl (2×100 mL), with brine. A precipitate appeared, which was filtered, washed with AcOEt followed by MeCN and dried under vacuum. The mother liquors were concentrated to an approximative volume of 30 mL. A precipitate appeared which was filtered, washed with AcOEt followed by MeCN and dried under vacuum. The gathered powders (7 g) were diluted in AcOEt and a 10% aqueous solution of NH₄OH. The organic layer was separated, dried over sodium sulfate, filtered and concentrated to dryness to give a brown solid which was then triturated in i-Pr₂O, filtered and dried under vacuum to give 4.088 g (58%) of [4-(2,3-diamino-4-pyridyl)-3,6-dihydro-2H-pyridin-1-yl]-[2-nitro-4-(trifluoromethoxy)phenyl]methanone as a brown solid. LC/MS (m/z, M+H): 424

### Step 6: (Rac)-tert-butyl 2-[7-[1-[2-nitro-4-(trifluoromethoxy)benzoyl]-3,6-dihydro-2H-pyridin-4-yl]-3H-imidazo[4,5-b]pyridin-2-yl]morpholine-4-carboxylate

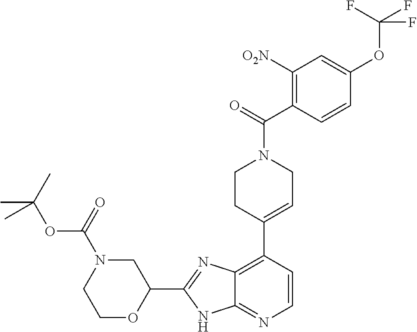 [SMILES: CC(C)(C)OC(=O)N1CCOC(c2nc3c(C4=CCN(C(=O)c5ccc(OC(F)(F)F)cc5[N+](=O)[O-])CC4)ccnc3[nH]2)C1]

Step 6 of Example 29 was prepared following a similar procedure to that of step 1 of Example 20 from (rac)-4-tert-butoxycarbonylmorpholine-2-carboxylic acid (197 mg, 0.85 mmol) with [4-(2,3-diamino-4-pyridyl)-3,6-dihydro-2H-pyridin-1-yl]-[2-nitro-4-(trifluoromethoxy)phenyl]methanone (300 mg, 0.71 mmol), CDI (161 mg, 0.99 mmol), in DMF/pyridine (1.5 mL/1.5 mL) to give 438 mg (77%) of tert-butyl 2-[7-[1-[2-nitro-4-(trifluoromethoxy)benzoyl]-3,6-dihydro-2H-pyridin-4-yl]-3H-imidazo[4,5-b]pyridin-2-yl]morpholine-4-carboxylate as a pale yellow solid. LC/MS (m/z, M+H): 619

### Step 7: (Rac)-tert-butyl 2-[7-[1-[2-amino-4-(trifluoromethoxy)benzoyl]-4-piperidyl]-3H-imidazo[4,5-b]pyridin-2-yl]morpholine-4-carboxylate

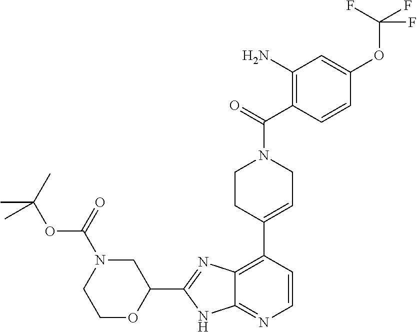 [SMILES: CC(C)(C)OC(=O)N1CCOC(c2nc3c(C4=CCN(C(=O)c5ccc(OC(F)(F)F)cc5N)CC4)ccnc3[nH]2)C1]

To a solution of (rac)-tert-butyl 2-[7-[1-[2-nitro-4-(trifluoromethoxy)benzoyl]-3,6-dihydro-2H-pyridin-4-yl]-3H-imidazo[4,5-b]pyridin-2-yl]morpholine-4-carboxylate (325 mg, 0.52 mmol) in MeOH (25 mL) was added ammonium formate (1325 mg, 21.01 mmol) and Pd/C (10%) (100 mg). The resulting mixture was then stirred at 65° C. for 1 hour. The reaction mixture was then cooled down to room temperature, filtered on Dicalite®, washed with MeOH and concentrated under vacuo. The resulting residue was then diluted with AcOEt and a 2N solution of NaOH. The organic layer was separated and concentrated. The resulting residue was then purified by flash chromatography (SiO₂ 12 g, eluting with DCM 95/MeOH 5/NH₄OH 0.5) to give 260 mg (83%) of (rac)-tert-butyl 2-[7-[1-[2-amino-4-(trifluoromethoxy)benzoyl]-4-piperidyl]-3H-imidazo[4,5-b]pyridin-2-yl]morpholine-4-carboxylate as a white powder. LC/MS (m/z, M+H): 591

### Step 8: (Rac)-tert-butyl 2-[7-[1-[2-amino-4-(trifluoromethoxy)benzoyl]-4-piperidyl]-3H-imidazo[4,5-b]pyridin-2-yl]morpholine-4-carboxylate

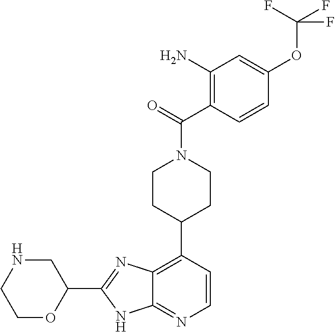 [SMILES: Nc1cc(OC(F)(F)F)ccc1C(=O)N1CCC(c2ccnc3[nH]c(C4CNCCO4)nc23)CC1]

Step 8 of Example 29 was prepared following a similar procedure to that of step 3 of Example 6 from (rac)-tert-butyl 2-[7-[1-[2-amino-4-(trifluoromethoxy)benzoyl]-4-piperidyl]-3H-imidazo[4,5-b]pyridin-2-yl]morpholine-4-carboxylate (256 mg, 0.43 mmol) with TFA (0.33 mL, 4.33 mmol) in DCM (2 mL) to give 194 mg (91%) of (rac)-tert-butyl 2-[7-[1-[2-amino-4-(trifluoromethoxy)benzoyl]-4-piperidyl]-3H-imidazo[4,5-b]pyridin-2-yl]morpholine-4-carboxylate as a white solid. LC/MS (m/z, M+H, Method 1): calc. 491, found 491.3; ¹H NMR (400 MHz, DMSO-d6, 30° C.) δ ppm 1.68-1.95 (m, 4H), 2.76-2.82 (m, 2H), 2.91 (br dd, J=12, 10 Hz, 1H), 2.98-3.40 (m, 3H), 3.46 (dtd, J=15, 8, 8, 4 Hz, 2H), 3.60-3.72 (m, 1H), 3.89 (br d, J=11 Hz, 1H), 3.92-4.56 (m, 2H), 4.68 (br dd, J=10, 2 Hz, 1H), 5.59 (s, 2H), 6.49 (br dd, J=8, 1 Hz, 1H), 6.66 (br d, J=1 Hz, 1H), 7.09 (d, J=5 Hz, 1H), 7.16 (d, J=8 Hz, 1H), 8.22 (d, J=5 Hz, 1H), 12.36-13.51 (m, 1H)

### Example 30: (Rac)-[4-[2-(2,2-dimethyltetrahydropyran-4-yl)-3H-imidazo[4,5-b]pyridin-7-yl]-1-piperidyl]-[4-(trifluoromethoxy)phenyl]methanone

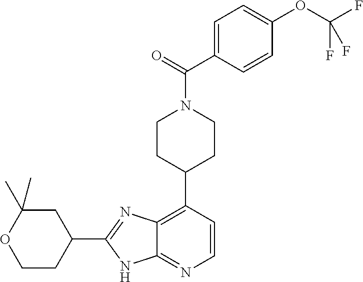 [SMILES: CC1(C)CC(c2nc3c(C4CCN(C(=O)c5ccc(OC(F)(F)F)cc5)CC4)ccnc3[nH]2)CCO1]

Example 30 was prepared following a similar procedure to that of step 1 of Example 20 from (rac)-2,2-dimethyltetrahydropyran-4-carboxylic acid (75 mg, 0.47 mmol) with [4-(2,3-diamino-4-pyridyl)-1-piperidyl]-[4-(trifluoromethoxy)phenyl]methanone (150 mg, 0.39 mmol), CDI (89 mg, 0.55 mmol), in DMF/pyridine (1.5 mL/1.5 mL) to give 155 mg (78%) of (rac)-[4-[2-(2,2-dimethyltetrahydropyran-4-yl)-3H-imidazo[4,5-b]pyridin-7-yl]-1-piperidyl]-[4-(trifluoromethoxy)phenyl]methanone as a pale brown solid. LC/MS (m/z, M+H, Method 1): calc. 503, found 503.3; ¹H NMR (400 MHz, DMSO-d6, 120° C.) δ ppm 1.20 (s, 3H), 1.28 (s, 3H), 1.66-2.01 (m, 8H), 3.07-3.18 (m, 2H), 3.29 (tt, J=12, 4 Hz, 1H), 3.39-3.50 (m, 1H), 3.68-3.86 (m, 2H), 4.09-4.23 (m, 2H), 7.00 (d, J=5 Hz, 1H), 7.37 (dd, J=9, 1 Hz, 2H), 7.56 (d, J=9 Hz, 2H), 8.14 (d, J=5 Hz, 1H), 11.33-13.20 (m, 1H)

### Example 31: (Rac)-[4-[2-(6,6-dimethyltetrahydropyran-3-yl)-3H-imidazo[4,5-b]pyridin-7-yl]-1-piperidyl]-[4-(trifluoromethoxy)phenyl]methanone

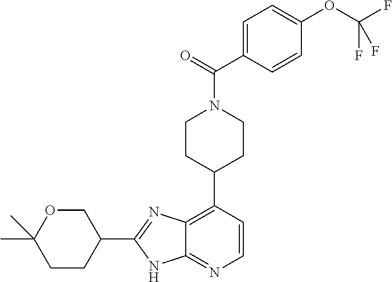 [SMILES: CC1(C)CCC(c2nc3c(C4CCN(C(=O)c5ccc(OC(F)(F)F)cc5)CC4)ccnc3[nH]2)CO1]

Example 31 was prepared following a similar procedure to that of step 1 of Example 20 from (rac)-6,6-dimethyltetrahydropyran-3-carboxylic acid (75 mg, 0.47 mmol) with [4-(2,3-diamino-4-pyridyl)-1-piperidyl]-[4-(trifluoromethoxy)phenyl]methanone (150 mg, 0.39 mmol), CDI (89 mg, 0.55 mmol), in DMF/pyridine (1.5 mL/1.5 mL) to give 176 mg (85%) of (rac)-[4-[2-(6,6-dimethyltetrahydropyran-3-yl)-3H-imidazo[4,5-b]pyridin-7-yl]-1-piperidyl]-[4-(trifluoromethoxy)phenyl]methanone as a pale brown solid. LC/MS (m/z, M+H), Method 1: calc. 503, found 503.3; ¹H NMR (400 MHz, DMSO-d6, 120° C.) δ ppm 1.21 (s, 3H), 1.24 (s, 3H), 1.49-1.60 (m, 1H), 1.68 (dt, J=13, 4 Hz, 1H), 1.83-1.99 (m, 4H), 1.99-2.18 (m, 2H), 3.02 (tt, J=10, 5 Hz, 1H), 3.06-3.18 (m, 2H), 3.37-3.48 (m, 1H), 3.82-3.96 (m, 2H), 4.09-4.25 (m, 2H), 7.01 (d, J=5 Hz, 1H), 7.37 (br d, J=9 Hz, 2H), 7.56 (br d, J=9 Hz, 2H), 8.15 (d, J=5 Hz, 1H), 11.47-13.20 (m, 1H)

### Examples 32 and 33: [4-[2-(1,4-Dioxan-2-yl)-3H-imidazo[4,5-b]pyridin-7-yl]-1-piperidyl]-[4-(trifluoromethoxy)phenyl]methanone, Isomers 1 and 2

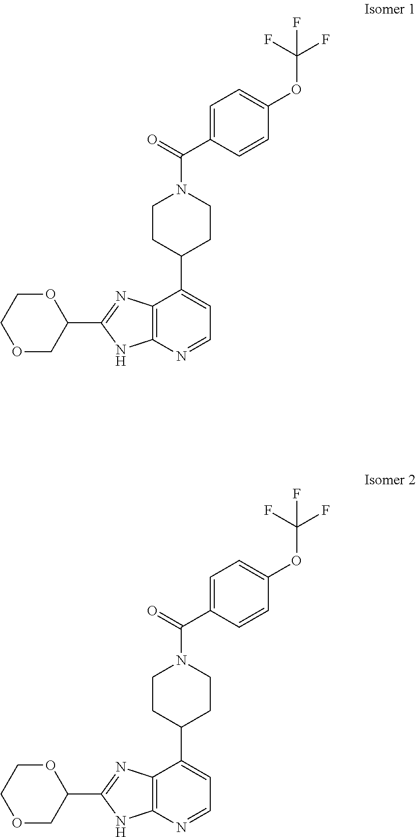 [SMILES: O=C(c1ccc(OC(F)(F)F)cc1)N1CCC(c2ccnc3[nH]c(C4COCCO4)nc23)CC1 ; O=C(c1ccc(OC(F)(F)F)cc1)N1CCC(c2ccnc3[nH]c(C4COCCO4)nc23)CC1]

### Step 1: (Rac)-[4-[2-(1,4-dioxan-2-yl)-3H-imidazo[4,5-b]pyridin-7-yl]-1-piperidyl]-[4-(trifluoromethoxy)phenyl]methanone

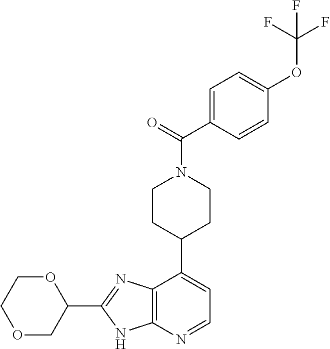 [SMILES: O=C(c1ccc(OC(F)(F)F)cc1)N1CCC(c2ccnc3[nH]c(C4COCCO4)nc23)CC1]

Step 1 of Examples 32 and 33 was prepared following a similar procedure to that of step 1 of Example 20 from 1,4-dioxane-2-carboxylic acid (63 mg, 0.46 mmol) with [4-(2,3-diamino-4-pyridyl)-1-piperidyl]-[4-(trifluoromethoxy)phenyl]methanone (125 mg, 0.33 mmol), CDI (60 mg, 0.58 mmol), in DMF/pyridine (1.25 mL/1.25 mL) to give 95 mg (61%) of (rac)-[4-[2-(1,4-dioxan-2-yl)-3H-imidazo[4,5-b]pyridin-7-yl]-1-piperidyl]-[4-(trifluoromethoxy)phenyl]methanone as a white solid. LC/MS (m/z, M+H): 477

### Step 2: [4-[2-(1,4-Dioxan-2-yl)-3H-imidazo[4,5-b]pyridin-7-yl]-1-piperidyl]-[4-(trifluoromethoxy)phenyl]methanone, Isomers 1 and 2

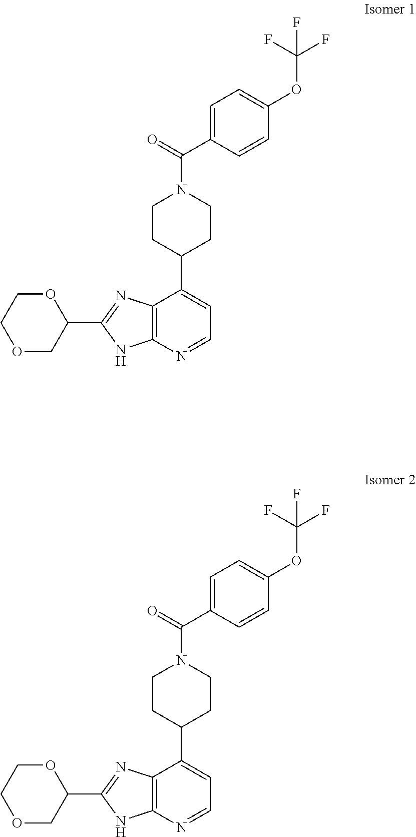 [SMILES: O=C(c1ccc(OC(F)(F)F)cc1)N1CCC(c2ccnc3[nH]c(C4COCCO4)nc23)CC1 ; O=C(c1ccc(OC(F)(F)F)cc1)N1CCC(c2ccnc3[nH]c(C4COCCO4)nc23)CC1]

Step 1 of Examples 32 and 33 consisted in the chiral separation of (rac)-[4-[2-(1,4-dioxan-2-yl)-3H-imidazo[4,5-b]pyridin-7-yl]-1-piperidyl]-[4-(trifluoromethoxy)phenyl]methanone (81 mg, 0.17 mmol), using an i.cellulose 5 column (250×30 mm), eluting with (n-Heptane 40% EtOH 60%)+0.1% TEA (flow rate 40 mL/min, UV detection at 265 nm). Pure fractions of each enantiomer were collected separately and concentrated to furnish 34 mg (42%) of the first eluting Isomer 1 (Example 32) and 36 mg (44%) of Isomer 2 (Example 33) as white solids.

Isomer 1 (Example 32): LC/MS (m/z, M+H, Method 2): calc. 477, found 477.2; ¹H NMR (400 MHz, DMSO-d6, 30° C.) δ ppm 1.71-2.07 (m, 4H), 2.87-3.07 (m, 1H), 3.11-3.35 (m partially hidden, 1H), 3.41-3.57 (m, 1H), 3.60-3.70 (m, 2H), 3.73-3.87 (m, 3H), 3.88-3.96 (m, 1H), 4.00-4.13 (m, 1H), 4.56-4.79 (m, 1H), 4.82-4.97 (m, 1H), 7.13 (d, J=5 Hz, 1H), 7.46 (br d, J=9 Hz, 2H), 7.60 (br d, J=9 Hz, 2H), 8.15-8.36 (m, 1H), 12.55-13.59 (m, 1H)

Isomer 2 (Example 33): LC/MS (m/z, M+H, Method 2): calc. 477, found 477.2; ¹H NMR (400 MHz, DMSO-d6, 30° C.) δ ppm 1.71-2.07 (m, 4H), 2.87-3.07 (m, 1H), 3.11-3.35 (m partially hidden, 1H), 3.41-3.57 (m, 1H), 3.60-3.70 (m, 2H), 3.73-3.87 (m, 3H), 3.88-3.96 (m, 1H), 4.00-4.13 (m, 1H), 4.56-4.79 (m, 1H), 4.82-4.97 (m, 1H), 7.13 (d, J=5 Hz, 1H), 7.46 (br d, J=9 Hz, 2H), 7.60 (br d, J=9 Hz, 2H), 8.15-8.36 (m, 1H), 12.55-13.59 (m, 1H)

### Example 34: (Rac)-[4-(2-tetrahydrofuran-2-yl-3H-imidazo[4,5-b]pyridin-7-yl)-1-piperidyl]-[4-(trifluoromethoxy)phenyl]methanone

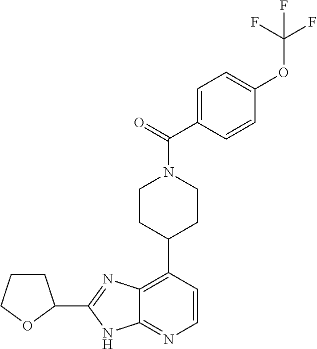 [SMILES: O=C(c1ccc(OC(F)(F)F)cc1)N1CCC(c2ccnc3[nH]c(C4CCCO4)nc23)CC1]

Example 34 was prepared following a similar procedure to that of step 3 of Examples 1 and 2 from [4-(2,3-diamino-4-pyridyl)-1-piperidyl]-[4-(trifluoromethoxy)phenyl]methanone (156 mg, 0.41 mmol) and tetrahydrofuran-2-carboxylic acid (50 mg, 0.43 mmol) with CDI (77 mg, 0.47 mmol) in acetonitrile (5 mL) to give 120 mg (59%) of (rac)-[4-(2-tetrahydrofuran-2-yl-3H-imidazo[4,5-b]pyridin-7-yl)-1-piperidyl]-[4-(trifluoromethoxy)phenyl]methanone as a white foam. LC/MS (m/z, M+H, Method 2): calc. 461, found 461.2; ¹H NMR (400 MHz, DMSO-d6, 100° C.) δ ppm 1.78-2.15 (m, 6H), 2.20-2.39 (m, 2H), 3.05-3.18 (m, 2H), 3.41-3.53 (m, 1H), 3.79-3.89 (m, 1H), 3.95-4.04 (m, 1H), 4.09-4.25 (m, 2H), 5.07 (t, J=7 Hz, 1H), 7.05 (d, J=5 Hz, 1H), 7.39 (d, J=9 Hz, 2H), 7.57 (d, J=9 Hz, 2H), 8.19 (d, J=5 Hz, 1H), 11.97-13.14 (m, 1H)

### Example 35: (Rac)-[4-[2-(4-methylmorpholin-2-yl)-3H-imidazo[4,5-b]pyridin-7-yl]-1-piperidyl]-[4-(trifluoromethoxy)phenyl]methanone

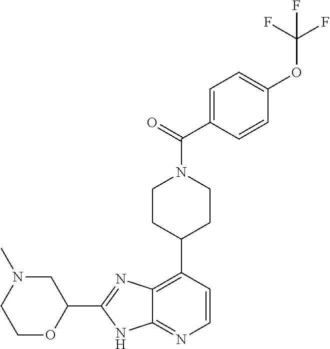 [SMILES: CN1CCOC(c2nc3c(C4CCN(C(=O)c5ccc(OC(F)(F)F)cc5)CC4)ccnc3[nH]2)C1]

To a solution of (rac)-4-methylmorpholine-2-carboxylic acid (24 mg, 0.16 mmol) in DMF/pyridine (0.5 mL/0.5 mL) was added CDI (30 mg, 0.18 mmol). The resulting mixture was stirred at 45° C. for 30 minutes. Then, [4-(2,3-diamino-4-pyridyl)-1-piperidyl]-[4-(trifluoromethoxy)phenyl]methanone (50 mg, 0.13 mmol) was added and the resulting mixture was stirred at 80° C. for 24 hours. To complete the reaction, 30 mg of CDI (0.08 mmol) were added followed by two additions of the same amount 4 hours and 20 hours later. After 6 additional hours under stirring at 80° C., the reaction mixture was cooled down to room temperature, transferred in a separating funnel containing 50 mL of water. The whole was extracted three times with AcOEt (3×30 mL). the combined organic layers were then washed with brine (20 mL), dried over sodium sulfate, filtered and concentrated in vacuo. The resulting residue was purified by flash chromatography (SiO₂ 10 g, eluting with DCM 97/MeOH 3) to give 18 mg (28%) of (rac)-[4-[2-(4-methylmorpholin-2-yl)-3H-imidazo[4,5-b]pyridin-7-yl]-1-piperidyl]-[4-(trifluoromethoxy)phenyl]methanone as a white powder. LC/MS (m/z, M+H, Method 2): calc. 490, found 490.3; ¹H NMR (400 MHz, DMSO-d6, 120° C.) δ ppm 1.71-2.02 (m, 4H), 2.22 (td, J=11, 4 Hz, 1H), 2.29 (s, 3H), 2.40 (dd, J=11, Hz, 1H), 2.63-2.70 (m, 1H), 3.04 (br d, J=11 Hz, 1H), 3.08-3.18 (m, 2H), 3.43-3.58 (m, 1H), 3.76 (td, J=11, 3 Hz, 1H), 3.96 (dt, J=11, 3 Hz, 1H), 4.13-4.22 (m, 2H), 4.78 (dd, J=10, 3 Hz, 1H), 7.05 (d, J=5 Hz, 1H), 7.37 (d, J=9 Hz, 2H), 7.56 (d, J=9 Hz, 2H), 8.20 (d, J=5 Hz, 1H), 11.89-12.97 (m, 1H)

### Example 36: (Rac)-[2-amino-4-(trifluoromethoxy)phenyl]-[4-[2-(5,5-difluoro-3-piperidyl)-3H-imidazo[4,5-b]pyridin-7-yl]-1-piperidyl]methanone

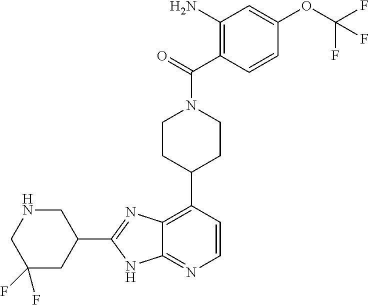 [SMILES: Nc1cc(OC(F)(F)F)ccc1C(=O)N1CCC(c2ccnc3[nH]c(C4CNCC(F)(F)C4)nc23)CC1]

### Step 1: (Rac)-tert-butyl 3,3-difluoro-5-[7-[1-[2-nitro-4-(trifluoromethoxy)benzoyl]-3,6-dihydro-2H-pyridin-4-yl]-3H-imidazo[4,5-b]pyridin-2-yl]piperidine-1-carboxylate

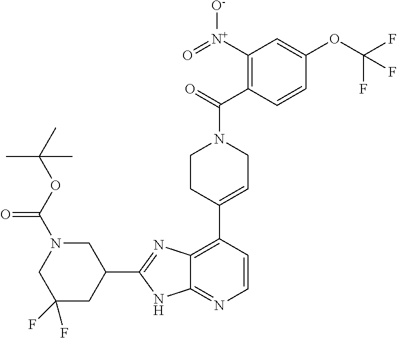 [SMILES: CC(C)(C)OC(=O)N1CC(c2nc3c(C4=CCN(C(=O)c5ccc(OC(F)(F)F)cc5[N+](=O)[O-])CC4)ccnc3[nH]2)CC(F)(F)C1]

Step 1 of Example 36 was prepared following a similar procedure to that of step 1 of Example 20 from (rac)-1-(tert-butoxycarbonyl)-5,5-difluoropiperidine-3-carboxylic acid (115 mg, 0.43 mmol) with [4-(2,3-diamino-4-pyridyl)-3,6-dihydro-2H-pyridin-1-yl]-[2-nitro-4-(trifluoromethoxy)phenyl]methanone (150 mg, 0.35 mmol), CDI (70 mg, 0.43 mmol), in DMF/pyridine (1 mL/1 mL) to give 149 mg (64%) of (rac)-tert-butyl 3,3-difluoro-5-[7-[1-[2-nitro-4-(trifluoromethoxy)benzoyl]-3,6-dihydro-2H-pyridin-4-yl]-3H-imidazo[4,5-b]pyridin-2-yl]piperidine-1-carboxylate as a pale yellow solid. LC/MS (m/z, M+H): 653

### Step 2: (Rac)-tert-butyl 5-[7-[1-[2-amino-4-(trifluoromethoxy)benzoyl]-4-piperidyl]-3H-imidazo[4,5-b]pyridin-2-yl]-3,3-difluoro-piperidine-1-carboxylate

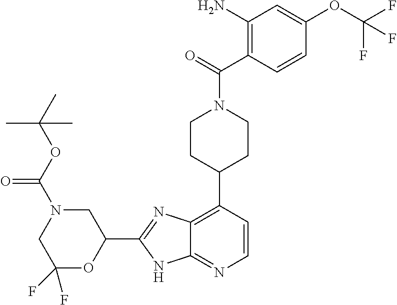 [SMILES: CC(C)(C)OC(=O)N1CC(c2nc3c(C4CCN(C(=O)c5ccc(OC(F)(F)F)cc5N)CC4)ccnc3[nH]2)OC(F)(F)C1]

Step 2 of Example 36 was prepared following a similar procedure to that of step 7 of Example 29 from (rac)-tert-butyl 3,3-difluoro-5-[7-[1-[2-nitro-4-(trifluoromethoxy)benzoyl]-3,6-dihydro-2H-pyridin-4-yl]-3H-imidazo[4,5-b]pyridin-2-yl]piperidine-1-carboxylate (145 mg, 0.22 mmol) with ammonium formate (280 mg, 4.44 mmol), Pd/C 10% (75 mg), in EtOH (6 mL) to give 44 mg (32%) of (rac)-tert-butyl 5-[7-[1-[2-amino-4-(trifluoromethoxy)benzoyl]-4-piperidyl]-3H-imidazo[4,5-b]pyridin-2-yl]-3,3-difluoro-piperidine-1-carboxylate as a white solid. LC/MS (m/z, M+H): 625

### Step 3: (Rac)-[2-amino-4-(trifluoromethoxy)phenyl]-[4-[2-(5,5-difluoro-3-piperidyl)-3H-imidazo[4,5-b]pyridin-7-yl]-1-piperidyl]methanone

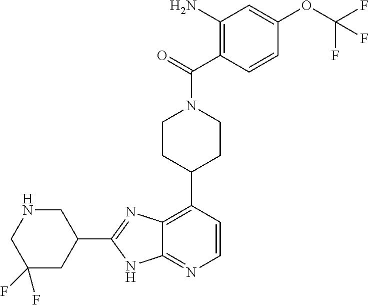 [SMILES: Nc1cc(OC(F)(F)F)ccc1C(=O)N1CCC(c2ccnc3[nH]c(C4CNCC(F)(F)C4)nc23)CC1]

To a solution of (rac)-tert-butyl 5-[7-[1-[2-amino-4-(trifluoromethoxy)benzoyl]-4-piperidyl]-3H-imidazo[4,5-b]pyridin-2-yl]-3,3-difluoro-piperidine-1-carboxylate (42 mg, 0.07 mmol) in DCM (3 mL) was added TFA (60 μL, 0.78 mmol). The resulting mixture was then stirred at room temperature for 4.5 hours. Additional TFA (60 μL, 0.78 mmol) was then added, and the resulting mixture was stirred for 12 hours at room temperature. The reaction mixture was then diluted with DCM and water. Solid NaHCO₃ was portionwise added until the pH was adjusted to 9. Then, the whole mixture was filtered on an hydrophobic cartridge (70 mL). The resulting organic layer was concentrated under reduced pressure to give 24 mg (68%) of (rac)-[2-amino-4-(trifluoromethoxy)phenyl]-[4-[2-(5,5-difluoro-3-piperidyl)-3H-imidazo[4,5-b]pyridin-7-yl]-1-piperidyl]methanone as a white powder. LC/MS (m/z, M+H, Method 1): calc. 525, found 525.3; ¹H NMR (400 MHz, DMSO-d6, 120° C.) δ ppm 1.72-2.05 (m, 4H), 2.22-2.60 (m partially hidden, 2H), 2.73-2.97 (m partially hidden, 2H), 3.04-3.17 (m, 3H), 3.22-3.32 (m, 2H), 3.36-3.52 (m, 1H), 4.16 (br d, J=12 Hz, 2H), 5.29 (br s, 2H), 6.48 (br dd, J=8, 1 Hz, 1H), 6.68 (br d, J=1 Hz, 1H), 7.01 (d, J=5 Hz, 1H), 7.15 (d, J=8 Hz, 1H), 8.06-8.33 (m, 1H), 11.83-12.82 (m, 1H)

### Example 37: [2-Amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone

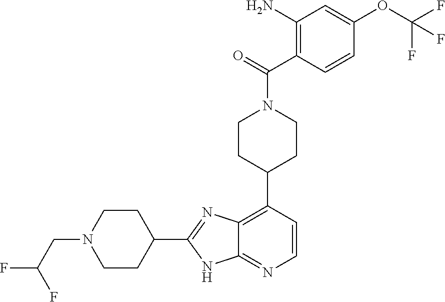 [SMILES: Nc1cc(OC(F)(F)F)ccc1C(=O)N1CCC(c2ccnc3[nH]c(C4CCN(CC(F)F)CC4)nc23)CC1]

### Step 1: [4-[2-[1-(2,2-Difluoroethyl)-4-piperidyl]-3H-imidazo[4,5-b]pyridin-7-yl]-3,6-dihydro-2H-pyridin-1-yl]-[2-nitro-4-(trifluoromethoxy)phenyl]methanone

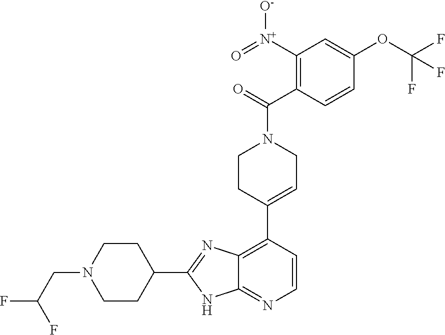 [SMILES: O=C(c1ccc(OC(F)(F)F)cc1[N+](=O)[O-])N1CC=C(c2ccnc3[nH]c(C4CCN(CC(F)F)CC4)nc23)CC1]

Step 1 of Example 37 was prepared following a similar procedure to that of step 1 of Example 20 from 1-(2,2-difluoroethyl)piperidine-4-carboxylic acid hydrochloride (65 mg, 0.28 mmol) with [4-(2,3-diamino-4-pyridyl)-3,6-dihydro-2H-pyridin-1-yl]-[2-nitro-4-(trifluoromethoxy)phenyl]methanone (100 mg, 0.24 mmol), CDI (45 mg, 0.28 mmol), in DMF/pyridine (1 mL/1 mL) to give 44 mg (32%) of [4-[2-[1-(2,2-difluoroethyl)-4-piperidyl]-3H-imidazo[4,5-b]pyridin-7-yl]-3,6-dihydro-2H-pyridin-1-yl]-[2-nitro-4-(trifluoromethoxy)phenyl]methanone as a pale yellow solid. LC/MS (m/z, M+H): 581

### Step 2: [2-Amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone

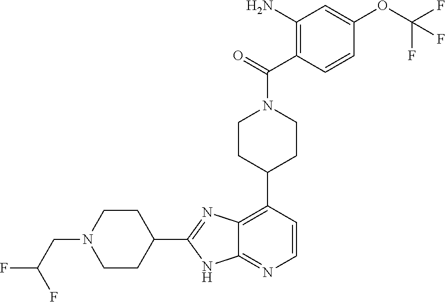 [SMILES: Nc1cc(OC(F)(F)F)ccc1C(=O)N1CCC(c2ccnc3[nH]c(C4CCN(CC(F)F)CC4)nc23)CC1]

A solution of [4-[2-[1-(2,2-difluoroethyl)-4-piperidyl]-3H-imidazo[4,5-b]pyridin-7-yl]-3,6-dihydro-2H-pyridin-1-yl]-[2-nitro-4-(trifluoromethoxy)phenyl]methanone (40 mg, 0.07 mmol) in MeOH (6 mL) was bubbled with argon for 5 minutes and then Pd/C 10% (50 mg) was added. The resulting mixture was then submitted to hydrogenation with 4 bars of H₂ at 50° C. for 2 hours. After 2 hours, the resulting mixture was cooled down to room temperature, filtered on GF/F microfiber filter, washed with MeOH. The filtrate was concentrated under reduced pressure and the resulting residue was purified by flash chromatography (SiO₂ 4 g, eluting with DCM/MeOH:100/0 to 95/5) to give 8 mg (210%) of [2-amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone as a white powder. LC/MS (m/z, M+H, Method 1): calc. 553, found 553.3; ¹H NMR (400 MHz, DMSO-d6, 30° C., mixture of conformer 70/30) δ ppm 1.69-2.04 (m, 8H), 2.22-2.39 (m, 2H), 2.70-2.90 (m, 3H), 2.94-3.17 (m, 4H), 3.37-3.55 (m, 2H), 3.75-4.72 (m, 2H), 5.57 (s, 1.4H), 5.61 (s, 0.6H), 6.14 (br tt, J=56, 4 Hz, 1H), 6.49 (br d, J=8 Hz, 1H), 6.66 (br s, 1H), 7.01-7.08 (m, 1H), 7.13-7.27 (m, 1H), 8.14 (d, J=5 Hz, 0.7H), 8.23 (d, J=5 Hz, 0.3H), 12.33 (s, 0.3H), 12.71 (s, 0.7H)

### Example 38: [4-[2-[1-(2,2-Difluoroethyl)azetidin-3-yl]-3H-imidazo[4,5-b]pyridin-7-yl]-1-piperidyl]-[4-(trifluoromethoxy)phenyl]methanone

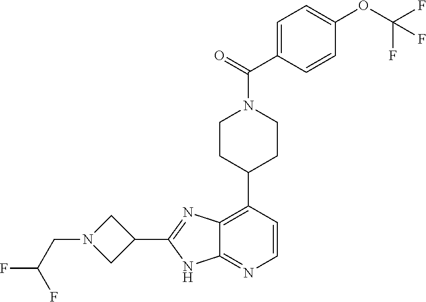 [SMILES: O=C(c1ccc(OC(F)(F)F)cc1)N1CCC(c2ccnc3[nH]c(C4CN(CC(F)F)C4)nc23)CC1]

### Step 1: [4-[2-(Azetidin-3-yl)-3H-imidazo[4,5-b]pyridin-7-yl]-1-piperidyl]-[4-(trifluoromethoxy)phenyl]methanone, hydrochloride

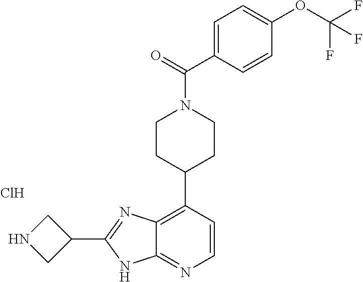 [SMILES: Cl ; O=C(c1ccc(OC(F)(F)F)cc1)N1CCC(c2ccnc3[nH]c(C4CNC4)nc23)CC1]

To a solution of tert-butyl 3-[7-[1-[4-(trifluoromethoxy)benzoyl]-4-piperidyl]-3H-imidazo[4,5-b]pyridin-2-yl]azetidine-1-carboxylate (295 mg, 0.54 mmol) in MeOH (6 mL) at 5° C. was dropwise added a 4N solution of HCl in 1,4-dioxane (1.5 mL, 6.0 mmol). The resulting mixture was then warmed up to room temperature and stirred for 12 hours. The reaction mixture was concentrated to dryness to give 134 mg (48%) of [4-[2-(azetidin-3-yl)-3H-imidazo[4,5-b]pyridin-7-yl]-1-piperidyl]-[4-(trifluoromethoxy)phenyl]methanone, hydrochloride as an orange gum. LC/MS (m/z, M+H): 519 (free base)

### Step 2: [4-[2-[1-(2,2-Difluoroethyl)azetidin-3-yl]-3H-imidazo[4,5-b]pyridin-7-yl]-1-piperidyl]-[4-(trifluoromethoxy)phenyl]methanone

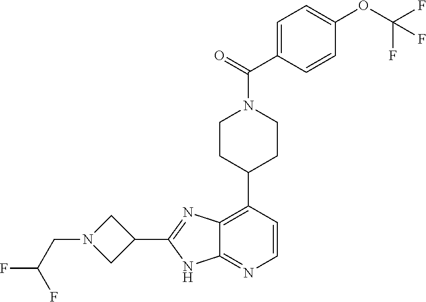 [SMILES: O=C(c1ccc(OC(F)(F)F)cc1)N1CCC(c2ccnc3[nH]c(C4CN(CC(F)F)C4)nc23)CC1]

To a solution of[4-[2-(azetidin-3-yl)-3H-imidazo[4,5-b]pyridin-7-yl]-1-piperidyl]-[4-(trifluoromethoxy)phenyl]methanone, hydrochloride (140 mg, 0.29 mmol) in acetonitrile (6 mL) was added potassium carbonate (230 mg, 1.66 mmol), followed by 2,2-difluoroethyl trifluoromethanesulfonate (120 mg, 0.56 mmol). The resulting reaction mixture was then stirred at 45° C. for 75 minutes. Then, the reaction mixture was cooled down to room temperature, concentrated to dryness, diluted with AcOEt (20 mL) and water (20 mL). The organic layer was separated, and the aqueous layer was extracted once with AcOEt (20 mL). The combined organic layers were then washed with brine, dried over sodium sulfate, filtered and concentrated under reduced pressure. The resulting residue was then purified by flash chromatography (SiO₂ 12 g, eluting with DCM/MeOH: 100/0 to 95/5) to give 41 mg (27%) of [4-[2-[1-(2,2-difluoroethyl)azetidin-3-yl]-3H-imidazo[4,5-b]pyridin-7-yl]-1-piperidyl]-[4-(trifluoromethoxy)phenyl]methanone as a white solid. LC/MS (m/z, M+H, Method 1): calc. 510, found 510.3; ¹H NMR (400 MHz, DMSO-d6, 30° C., mixture of conformer 70/30) δ ppm 1.67-2.07 (m, 4H), 2.91 (td, J=16, 4 Hz, 2H), 2.86-3.13 (m, 1H), 3.18-3.84 (m partially hidden, 3H), 3.55-3.64 (m, 2H), 3.75 (br t, J=7 Hz, 2H), 3.91 (quin, J=7 Hz, 1H), 4.54-4.85 (m, 1H), 5.99 (tt, J=56, 4 Hz, 1H), 7.10 (d, J=5 Hz, 1H), 7.45 (br d, J=8 Hz, 2H), 7.60 (br d, J=8 Hz, 2H), 8.14-8.20 (m, 0.7H), 8.20-8.36 (m, 0.3H), 12.49 (br s, 0.3H), 12.92 (s, 0.7H)

### Example 39: [4-[2-(Oxetan-3-ylmethyl)-3H-imidazo[4,5-b]pyridin-7-yl]-1-piperidyl]-[4-(trifluoromethoxy)phenyl]methanone

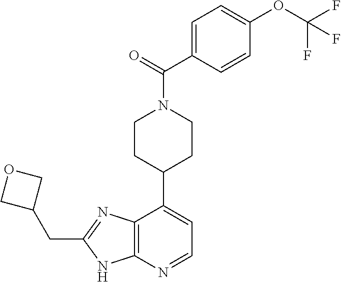 [SMILES: O=C(c1ccc(OC(F)(F)F)cc1)N1CCC(c2ccnc3[nH]c(CC4COC4)nc23)CC1]

Example 39 was prepared following a similar procedure to that of step 1 of Example 15 from 1 2-(oxetan-3-yl)acetic acid (59 mg, 0.50 mmol) with [4-(2,3-diamino-4-pyridyl)-1-piperidyl]-[4-(trifluoromethoxy)phenyl]methanone (133 mg, 0.35 mmol), CDI (85 mg, 0.53 mmol), in DMF (1.5 mL) to give 105 mg (65%) of [4-[2-(oxetan-3-ylmethyl)-3H-imidazo[4,5-b]pyridin-7-yl]-1-piperidyl]-[4-(trifluoromethoxy)phenyl]methanone as a pale yellow solid. LC/MS (m/z, M+H, Method 1): calc. 461, found 461.2; ¹H NMR (400 MHz, DMSO-d6, 100° C.) δ ppm 1.80-2.00 (m, 4H), 3.05-3.16 (m, 2H), 3.20 (d, J=8 Hz, 2H), 3.31-3.43 (m, 1H), 3.44-3.61 (m, 1H), 4.02-4.29 (m, 2H), 4.43 (t, J=6 Hz, 2H), 4.72 (dd, J=8, 6 Hz, 2H), 7.01 (d, J=5 Hz, 1H), 7.39 (br d, J=9 Hz, 2H), 7.57 (d, J=9 Hz, 2H), 8.14 (d, J=5 Hz, 1H), 11.33-13.11 (m, 1H)

### Example 40: (4-Chloro-1H-indol-7-yl)-[4-(2-tetrahydropyran-4-yl-3H-imidazo[4,5-b]pyridin-7-yl)-1-piperidyl]methanone

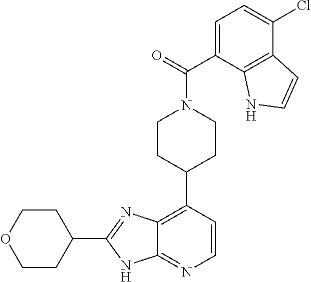 [SMILES: O=C(c1ccc(Cl)c2cc[nH]c12)N1CCC(c2ccnc3[nH]c(C4CCOCC4)nc23)CC1]

Example 40 was prepared following a similar procedure to that of step 5 of Examples 1 and 2 from 7-(4-piperidyl)-2-tetrahydropyran-4-yl-3H-imidazo[4,5-b]pyridine, hydrochloride (100 mg, 0.28 mmol) and 4-chloro-1H-indole-7-carboxylic acid (57 mg, 0.29 mmol) with TATU (103 mg, 0.32 mmol) and DIPEA (0.24 mL, 1.39 mmol) in DMF (2 mL) to give 64 mg (50%) of (4-chloro-1H-indol-7-yl)-[4-(2-tetrahydropyran-4-yl-3H-imidazo[4,5-b]pyridin-7-yl)-1-piperidyl]methanone as a white solid. LC/MS (m/z, M+H, Method 1): calc. 464, found 464.2; ¹H NMR (400 MHz, DMSO-d6, 100° C.) δ ppm 1.81-2.04 (m, 8H), 3.10-3.22 (m, 3H), 3.37-3.72 (m, 3H), 3.91-4.00 (m, 2H), 4.22 (br d, J=12 Hz, 2H), 6.53 (dd, J=3, 2 Hz, 1H), 7.03 (d, J=5 Hz, 1H), 7.06-7.17 (m, 2H), 7.42 (t, J=3 Hz, 1H), 8.15 (br s, 1H), 11.16 (br s, 1H), 12.28-12.63 (m, 1H)

### Example 41: [2-Nitro-4-(pentafluoro-λ⁶-sulfanyl)phenyl]-[4-(2-tetrahydropyran-4-yl-3H-imidazo[4,5-b]pyridin-7-yl)-1-piperidyl]methanone

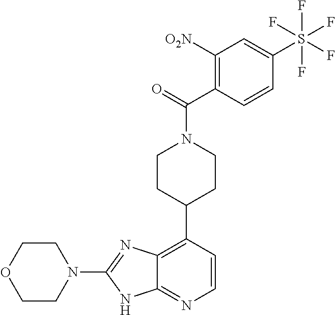 [SMILES: O=C(c1ccc(S(F)(F)(F)(F)F)cc1[N+](=O)[O-])N1CCC(c2ccnc3[nH]c(N4CCOCC4)nc23)CC1]

Example 41 was prepared following a similar procedure to that of step 5 of Examples 1 and 2 from 7-(4-piperidyl)-2-tetrahydropyran-4-yl-3H-imidazo[4,5-b]pyridine (50 mg, 0.17 mmol) and 2-nitro-4-(pentafluoro-λ⁶-sulfanyl)benzoic acid (46 mg, 0.17 mmol) with HATU (76 mg, 0.20 mmol) and DIPEA (0.07 mL, 0.40 mmol) in DMF (0.8 mL) to give 31 mg (22%) of [2-nitro-4-(pentafluoro-λ⁶-sulfanyl)phenyl]-[4-(2-tetrahydropyran-4-yl-3H-imidazo[4,5-b]pyridin-7-yl)-1-piperidyl]methanone as a white solid. LC/MS (m/z, M+H, Method 2): calc. 562, found 562.1; ¹H NMR (400 MHz, DMSO-d6, 101° C.) δ ppm 1.81-2.17 (m, 8H), 2.98-3.10 (m, 1H), 3.15 (tt, J=10, 5 Hz, 1H), 3.21-3.37 (m, 1H), 3.40-3.60 (m, 4H), 3.95 (dt, J=11, 3 Hz, 2H), 4.51-4.78 (m, 1H), 7.00 (d, J=5 Hz, 1H), 7.85 (d, J=8 Hz, 1H), 8.17 (br d, J=5 Hz, 1H), 8.34 (dd, J=8, 2 Hz, 1H), 8.56 (d, J=2 Hz, 1H), 11.75-12.98 (m, 1H)

### Example 42: (Rac)-[4-[2-(tetrahydrofuran-3-ylmethyl)-3H-imidazo[4,5-b]pyridin-7-yl]-1-piperidyl]-[4-(trifluoromethoxy)phenyl]methanone

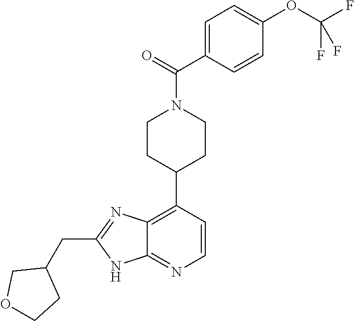 [SMILES: O=C(c1ccc(OC(F)(F)F)cc1)N1CCC(c2ccnc3[nH]c(CC4CCOC4)nc23)CC1]

Example 42 was prepared following a similar procedure to that of step 1 of Example 20 from 2-tetrahydrofuran-3-ylacetic acid (62 mg, 0.47 mmol) with [4-(2,3-diamino-4-pyridyl)-1-piperidyl]-[4-(trifluoromethoxy)phenyl]methanone (150 mg, 0.39 mmol), CDI (89 mg, 0.55 mmol), in DMF/pyridine (1.5 mL/1.5 mL) to give 104 mg (55%) of (rac)-[4-[2-(tetrahydrofuran-3-ylmethyl)-3H-imidazo[4,5-b]pyridin-7-yl]-1-piperidyl]-[4-(trifluoromethoxy)phenyl]methanone as a pale brown solid. LC/MS (m/z, M+H, Method 1): calc. 475, found 475.2; ¹H NMR (400 MHz, DMSO-d6, 101° C.) δ ppm 1.58-1.72 (m, 1H), 1.80-1.98 (m, 4H), 1.98-2.10 (m, 1H), 2.68-2.82 (m, 1H), 2.91 (d, J=7 Hz, 2H), 3.06-3.20 (m, 2H), 3.33-3.44 (m, 1H), 3.46 (dd, J=8, 6 Hz, 1H), 3.67 (q, J=8 Hz, 1H), 3.73-3.90 (m, 2H), 4.03-4.30 (m, 2H), 7.00 (d, J=5 Hz, 1H), 7.38 (d, J=8 Hz, 2H), 7.56 (d, J=8 Hz, 2H), 8.14 (d, J=5 Hz, 1H), 11.42-13.64 (m, 1H)

### Example 43: (Rac)-[4-[2-(oxepan-4-yl)-3H-imidazo[4,5-b]pyridin-7-yl]-1-piperidyl]-[4-(trifluoromethoxy)phenyl]methanone

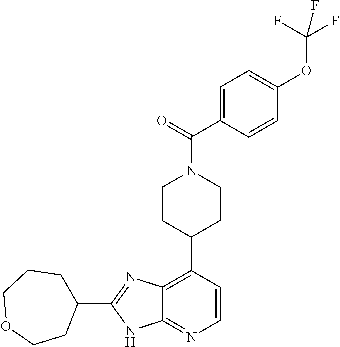 [SMILES: O=C(c1ccc(OC(F)(F)F)cc1)N1CCC(c2ccnc3[nH]c(C4CCCOCC4)nc23)CC1]

### Step 1: (Rac)-tert-butyl 4-[2-(oxepan-4-yl)-3H-imidazo[4,5-b]pyridin-7-yl]piperidine-1-carboxylate

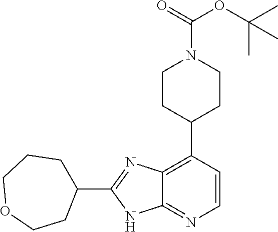 [SMILES: CC(C)(C)OC(=O)N1CCC(c2ccnc3[nH]c(C4CCCOCC4)nc23)CC1]

Step 1 of Example 43 was prepared following a similar procedure to that of step 1 of Example 5 from tert-butyl 4-(2,3-diamino-4-pyridyl)piperidine-1-carboxylate (250 mg, 0.85 mmol) and oxepane-4-carboxylic acid (191 mg, 1.33 mmol) with CDI (256 mg, 1.58 mmol) in acetonitrile (8 mL) under reflux for 24 hours to give 264 mg (77%) of (rac)-tert-butyl 4-[2-(oxepan-4-yl)-3H-imidazo[4,5-b]pyridin-7-yl]piperidine-1-carboxylate as a white solid. LC/MS (m/z, M+H): 401

### Step 2: (Rac)-2-(oxepan-4-yl)-7-(4-piperidyl)-3H-imidazo[4,5-b]pyridine, hydrochloride

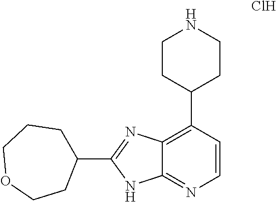 [SMILES: Cl ; c1cc(C2CCNCC2)c2nc(C3CCCOCC3)[nH]c2n1]

Step 2 of Example 43 was prepared following a similar procedure to that of step 4 of Examples 1 and 2 from (rac)-tert-butyl 4-[2-(oxepan-4-yl)-3H-imidazo[4,5-b]pyridin-7-yl]piperidine-1-carboxylate (109 mg, 0.27 mmol) using HCl 4N solution in 1,4-dioxane (0.68 mL, 2.72 mmol) in MeOH (2 mL) to give 92 mg (100%) of (rac)-2-(oxepan-4-yl)-7-(4-piperidyl)-3H-imidazo[4,5-b]pyridine, hydrochloride as an orange gum. LC/MS (m/z, M+H): 301 (free base)

### Step 3: (Rac)-[4-[2-(oxepan-4-yl)-3H-imidazo[4,5-b]pyridin-7-yl]-1-piperidyl]-[4-(trifluoromethoxy)phenyl]methanone

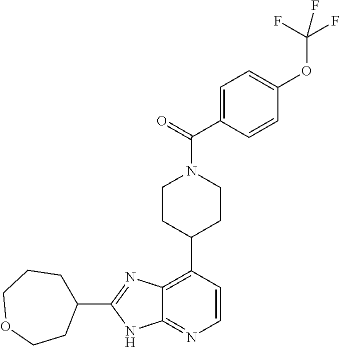 [SMILES: O=C(c1ccc(OC(F)(F)F)cc1)N1CCC(c2ccnc3[nH]c(C4CCCOCC4)nc23)CC1]

Step 3 of Example 43 was prepared following a similar procedure to that of step 5 of Examples 1 and 2 from 2-(oxepan-4-yl)-7-(4-piperidyl)-3H-imidazo[4,5-b]pyridine, hydrochloride (79 mg, 0.24 mmol) and 4-(trifluoromethoxy)benzoic acid (53 mg, 0.26 mmol) with HATU (108 mg, 0.28 mmol) and DIPEA (0.2 mL, 1.18 mmol) in DMF (5 mL) to give 22 mg (19%) of (rac)-[4-[2-(oxepan-4-yl)-3H-imidazo[4,5-b]pyridin-7-yl]-1-piperidyl]-[4-(trifluoromethoxy)phenyl]methanone as a white solid. LC/MS (m/z, M+H, Method 1): calc. 489, found 489.2; ¹H NMR (400 MHz, DMSO-d6, 100° C.) δ ppm 1.65-2.22 (m, 10H), 3.05-3.27 (m, 3H), 3.33-3.49 (m, 1H), 3.57-3.91 (m, 4H), 4.05-4.36 (m, 2H), 7.01 (d, J=5 Hz, 1H), 7.39 (br d, J=8 Hz, 2H), 7.57 (d, J=8 Hz, 2H), 8.14 (br d, J=5 Hz, 1H), 11.79-12.67 (m, 1H)

### Example 44: (Rac)-[2-amino-4-(trifluoromethoxy)phenyl]-[4-[2-(oxepan-4-yl)-3H-imidazo[4,5-b]pyridin-7-yl]-1-piperidyl]methanone

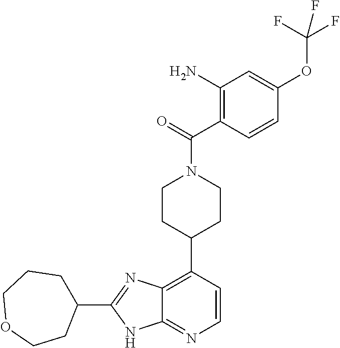 [SMILES: Nc1cc(OC(F)(F)F)ccc1C(=O)N1CCC(c2ccnc3[nH]c(C4CCCOCC4)nc23)CC1]

Example 44 was prepared following a similar procedure to that of step 5 of Examples 1 and 2 from 2-(oxepan-4-yl)-7-(4-piperidyl)-3H-imidazo[4,5-b]pyridine, hydrochloride (79 mg, 0.24 mmol) and 2-amino-4-(trifluoromethoxy)benzoic acid (57 mg, 0.26 mmol) with HATU (108 mg, 0.28 mmol) and DIPEA (0.2 mL, 1.18 mmol) in DMF (5 mL) to give 25 mg (21%) of (rac)-[2-amino-4-(trifluoromethoxy)phenyl]-[4-[2-(oxepan-4-yl)-3H-imidazo[4,5-b]pyridin-7-yl]-1-piperidyl]methanone as a white solid. LC/MS (m/z, M−H, Method 1): calc. 502, found 502.1; ¹H NMR (400 MHz, DMSO-d6, 100° C.) δ ppm 1.64-2.19 (m, 10H), 3.01-3.27 (m, 3H), 3.32-3.51 (m, 1H), 3.56-3.91 (m, 4H), 4.16 (br d, J=13 Hz, 2H), 5.35 (br s, 2H), 6.48 (br d, J=8 Hz, 1H), 6.68 (br s, 1H), 6.99 (d, J=5 Hz, 1H), 7.15 (d, J=8 Hz, 1H), 8.05-8.40 (m, 1H), 11.8-12.6 (m, 1H)

### Example 45: [4-[2-(2-Oxaspiro[3.3]heptan-6-ylmethyl)-3H-imidazo[4,5-b]pyridin-7-yl]-1-piperidyl]-[4-(trifluoromethoxy)phenyl]methanone

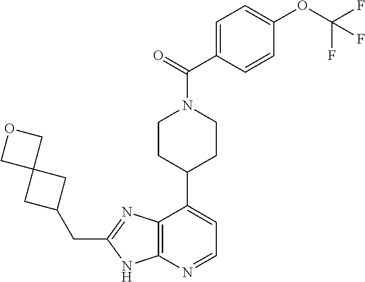 [SMILES: O=C(c1ccc(OC(F)(F)F)cc1)N1CCC(c2ccnc3[nH]c(CC4CC5(COC5)C4)nc23)CC1]

Example 45 was prepared following a similar procedure to that of step 1 of Example 20 from 2-(2-oxaspiro[3.3]heptan-6-yl)acetic acid (64 mg, 0.41 mmol) with [4-(2,3-diamino-4-pyridyl)-1-piperidyl]-[4-(trifluoromethoxy)phenyl]methanone (120 mg, 0.31 mmol), CDI (76 mg, 0.47 mmol), in DMF/pyridine (1.5 mL/1.5 mL) to give 57 mg (36%) of [4-[2-(2-oxaspiro[3.3]heptan-6-ylmethyl)-3H-imidazo[4,5-b]pyridin-7-yl]-1-piperidyl]-[4-(trifluoromethoxy)phenyl]methanone as a pale brown solid. LC/MS (m/z, M+H, Method 1): calc. 501, found 501.3; ¹H NMR (400 MHz, DMSO-d6, 100° C.) δ ppm 1.75-1.97 (m, 4H), 1.97-2.08 (m, 2H), 2.30-2.41 (m, 2H), 2.52-2.65 (m, 1H), 2.86 (d, J=7 Hz, 2H), 3.05-3.21 (m, 2H), 3.32-3.51 (m, 1H), 4.06-4.30 (m, 2H), 4.44 (s, 2H), 4.55 (s, 2H), 7.00 (d, J=5 Hz, 1H), 7.39 (br d, J=9 Hz, 2H), 7.57 (d, J=9 Hz, 2H), 8.13 (d, J=5 Hz, 1H), 11.70-12.71 (m, 1H)

### Example 46: [4-(1,1,2,2,2-Pentafluoroethyl)phenyl]-[4-(2-tetrahydropyran-4-yl-3H-imidazo[4,5-b]pyridin-7-yl)-1-piperidyl]methanone

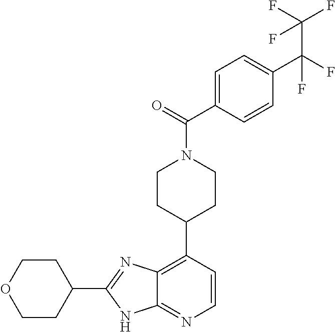 [SMILES: O=C(c1ccc(C(F)(F)C(F)(F)F)cc1)N1CCC(c2ccnc3[nH]c(C4CCOCC4)nc23)CC1]

Example 46 was prepared following a similar procedure to that of step 5 of Examples 1 and 2 from 7-(4-piperidyl)-2-tetrahydropyran-4-yl-3H-imidazo[4,5-b]pyridine, hydrochloride (100 mg, 0.28 mmol) and 4-(1,1,2,2,2-pentafluoroethyl)benzoic acid (70 mg, 0.29 mmol) with TATU (103 mg, 0.32 mmol) and DIPEA (0.24 mL, 1.39 mmol) in DMF (2 mL) to give 55 mg (39%) of [4-(1,1,2,2,2-pentafluoroethyl)phenyl]-[4-(2-tetrahydropyran-4-yl-3H-imidazo[4,5-b]pyridin-7-yl)-1-piperidyl]methanone as a yellow solid. LC/MS (m/z, M+H, Method 1): calc. 509, found 509; ¹H NMR (400 MHz, DMSO-d6, 30° C., mixture of conformer 75/25) δ ppm 1.64-2.11 (m, 8H), 2.84-3.07 (m, 1H), 3.07-3.20 (m, 1H), 3.25-3.38 (m hidden, 1H), 3.41-3.73 (m, 4H), 3.89-4.07 (m, 2H), 4.58-4.82 (m, 1H), 7.09 (d, J=5 Hz, 1H), 7.63-7.75 (m, 2H), 7.78-7.82 (m, 2H), 8.16 (d, J=5 Hz, 0.75H), 8.25 (d, J=5 Hz, 0.25H), 12.38 (s, 0.25H), 12.84 (s, 0.75H)

### Example 47: [4-(Pentafluoro-λ⁶-sulfanyl)phenyl]-[4-(2-tetrahydropyran-4-yl-3H-imidazo[4,5-b]pyridin-7-yl)-1-piperidyl]methanone

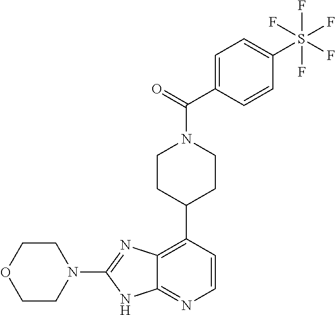 [SMILES: O=C(c1ccc(S(F)(F)(F)(F)F)cc1)N1CCC(c2ccnc3[nH]c(N4CCOCC4)nc23)CC1]

Example 47 was prepared following a similar procedure to that of step 5 of Examples 1 and 2 from 7-(4-piperidyl)-2-tetrahydropyran-4-yl-3H-imidazo[4,5-b]pyridine (100 mg, 0.35 mmol) and 4-(pentafluoro-λ⁶-sulfanyl)benzoic acid (86 mg, 0.34 mmol) with HATU (152 mg, 0.40 mmol) and DIPEA (0.22 mL, 1.27 mmol) in DMF (1.6 mL) to give 54 mg (25%) of [[4-(pentafluoro-λ⁶-sulfanyl)phenyl]-[4-(2-tetrahydropyran-4-yl-3H-imidazo[4,5-b]pyridin-7-yl)-1-piperidyl]methanone as a white solid. LC/MS (m/z, M+H, Method 2): calc. 517, found 517.1; ¹H NMR (400 MHz, DMSO-d6, 30° C., mixture of conformer 70/30) δ ppm 1.63-2.09 (m, 8H), 2.85-3.04 (m, 1H), 3.07-3.33 (m partially hidden, 2H), 3.41-3.69 (m, 4H), 3.88-4.03 (m, 2H), 4.55-4.84 (m, 1H), 7.09 (d, J=5 Hz, 1H), 7.64-7.75 (m, 2H), 8.00 (d, J=8 Hz, 2H), 8.16 (d, J=5 Hz, 0.7H), 8.25 (br d, J=5 Hz, 0.3H), 12.38 (s, 0.3H), 12.77 (s, 0.7H)

### Examples 48 and 49: (Cis)-[4-(2-tetrahydropyran-4-yl-3H-imidazo[4,5-b]pyridin-7-yl)-1-piperidyl]-[4-(2,2,2-trifluoroethyl)cyclohexyl]methanone and (trans)-[4-(2-tetrahydropyran-4-yl-3H-imidazo[4,5-b]pyridin-7-yl)-1-piperidyl]-[4-(2,2,2-trifluoroethyl)cyclohexyl]methanone

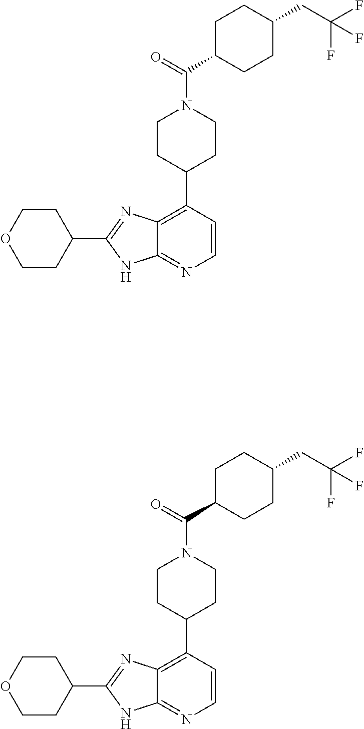 [SMILES: O=C([C@H]1CC[C@@H](CC(F)(F)F)CC1)N1CCC(c2ccnc3[nH]c(C4CCOCC4)nc23)CC1 ; O=C([C@H]1CC[C@H](CC(F)(F)F)CC1)N1CCC(c2ccnc3[nH]c(C4CCOCC4)nc23)CC1]

### Step 1: [4-(2-Tetrahydropyran-4-yl-3H-imidazo[4,5-b]pyridin-7-yl)-1-piperidyl]-[4-(2,2,2-trifluoroethyl)cyclohexyl]methanone

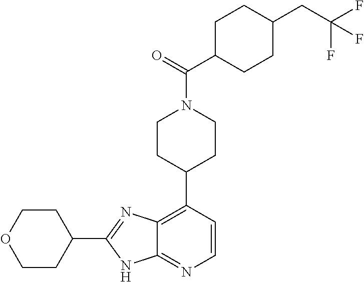 [SMILES: O=C(C1CCC(CC(F)(F)F)CC1)N1CCC(c2ccnc3[nH]c(C4CCOCC4)nc23)CC1]

Step 1 of Examples 48 and 49 was prepared following a similar procedure to that of step 5 of Examples 1 and 2 from 7-(4-piperidyl)-2-tetrahydropyran-4-yl-3H-imidazo[4,5-b]pyridine, hydrochloride (150 mg, 0.42 mmol) and 4-(2,2,2-trifluoroethyl)cyclohexane-1-carboxylic acid (92 mg, 0.44 mmol) with HATU (175 mg, 0.46 mmol) and DIPEA (0.36 mL, 2.09 mmol) in DMF (2.5 mL) to give 130 mg (65%) of [4-(2-tetrahydropyran-4-yl-3H-imidazo[4,5-b]pyridin-7-yl)-1-piperidyl]-[4-(2,2,2-trifluoroethyl)cyclohexyl]methanone as a white solid. LC/MS (m/z, M+H): 479

### Step 2: (Cis)-[4-(2-tetrahydropyran-4-yl-3H-imidazo[4,5-b]pyridin-7-yl)-1-piperidyl]-[4-(2,2,2-trifluoroethyl)cyclohexyl]methanone and (trans)-[4-(2-tetrahydropyran-4-yl-3H-imidazo[4,5-b]pyridin-7-yl)-1-piperidyl]-[4-(2,2,2-trifluoroethyl)cyclohexyl]methanone

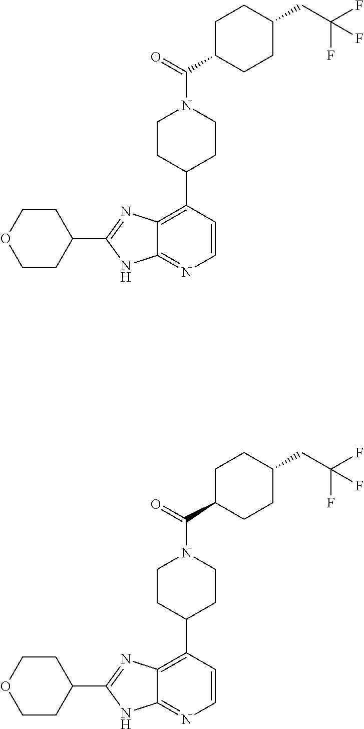 [SMILES: O=C([C@H]1CC[C@@H](CC(F)(F)F)CC1)N1CCC(c2ccnc3[nH]c(C4CCOCC4)nc23)CC1 ; O=C([C@H]1CC[C@H](CC(F)(F)F)CC1)N1CCC(c2ccnc3[nH]c(C4CCOCC4)nc23)CC1]

Examples 48 and 49 were obtained by separation of [4-(2-tetrahydropyran-4-yl-3H-imidazo[4,5-b]pyridin-7-yl)-1-piperidyl]-[4-(2,2,2-trifluoroethyl)cyclohexyl]methanone (122 mg, 0.25 mmol), using an i.cellulose 5 column (250×30 mm), eluting with (n-Heptane 70% EtOH 30%)+0.1% TEA (flow rate 45 mL/min, UV Detection at 265 nm). Pure fractions of each isomer were collected separately and concentrated to furnish 74 mg (61%) of the cis isomer and 15 mg (12%) of the trans isomer as white solids.

Cis-isomer (Example 48): LC/MS (m/z, M+H, Method 2): calc. 479, found 479.2; ¹H NMR (400 MHz, DMSO-d6, 101° C.) δ ppm 1.45-1.86 (m, 10H), 1.87-2.00 (m, 7H), 2.22 (qd, J=12, 7 Hz, 2H), 2.70-2.82 (m, 1H), 2.84-3.04 (m partially hidden, 2H), 3.14 (tt, J=10, 5 Hz, 1H), 3.31-3.41 (m, 1H), 3.49 (td, J=11, 4 Hz, 2H), 3.95 (dt, J=11, 3 Hz, 2H), 4.19-4.47 (m, 2H), 6.96 (d, J=5 Hz, 1H), 8.13 (d, J=5 Hz, 1H), 11.75-12.92 (m, 1H)

Trans-isomer (Example 49): LC/MS (m/z, M+H, Method 2): calc. 479, found 479.2; ¹H NMR (400 MHz, DMSO-d6, 100° C.) δ ppm 1.18 (qd, J=14, 3 Hz, 2H), 1.46 (qd, J=14, 3 Hz, 2H), 1.56-1.69 (m, 1H), 1.69-1.88 (m, 6H), 1.88-2.01 (m, 6H), 2.13 (qd, J=12, 7 Hz, 2H), 2.59 (tt, J=12, 3 Hz, 1H), 2.70-3.03 (m hidden, 2H), 3.14 (tt, J=10, 5 Hz, 1H), 3.27-3.42 (m, 1H), 3.43-3.60 (m, 2H), 3.95 (dt, J=11, 3 Hz, 2H), 4.20-4.52 (m, 2H), 6.96 (d, J=5 Hz, 1H), 8.13 (d, J=5 Hz, 1H), 11.89-12.78 (m, 1H)

### Example 50: [2-Amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone

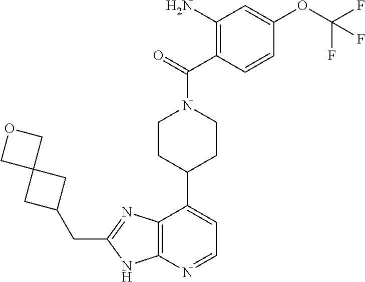 [SMILES: Nc1cc(OC(F)(F)F)ccc1C(=O)N1CCC(c2ccnc3[nH]c(CC4CC5(COC5)C4)nc23)CC1]

### Step 1: [2-Nitro-4-(trifluoromethoxy)phenyl]-[4-[2-(2-oxaspiro[3.3]heptan-6-ylmethyl)-3H-imidazo[4,5-b]pyridin-7-yl]-3,6-dihydro-2H-pyridin-1-yl]methanone

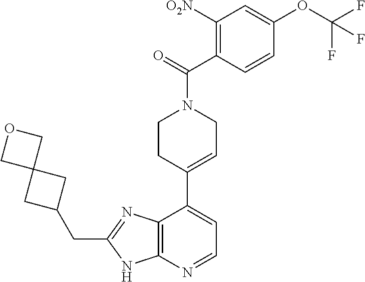 [SMILES: O=C(c1ccc(OC(F)(F)F)cc1[N+](=O)[O-])N1CC=C(c2ccnc3[nH]c(CC4CC5(COC5)C4)nc23)CC1]

Step 1 of Example 50 was prepared following a similar procedure to that of step 1 of Example 20 from 2-(2-oxaspiro[3.3]heptan-6-yl)acetic acid (109 mg, 0.70 mmol) with [4-(2,3-diamino-4-pyridyl)-3,6-dihydro-2H-pyridin-1-yl]-[2-nitro-4-(trifluoromethoxy)phenyl]methanone (260 mg, 0.58 mmol), CDI (132 mg, 0.82 mmol), in DMF/pyridine (1.5 mL/1.5 mL) to give 129 mg (39%) of [2-nitro-4-(trifluoromethoxy)phenyl]-[4-[2-(2-oxaspiro[3.3]heptan-6-ylmethyl)-3H-imidazo[4,5-b]pyridin-7-yl]-3,6-dihydro-2H-pyridin-1-yl]methanone as a pale brown solid. LC/MS (m/z, M+H): 544

### Step 2: [2-Amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone

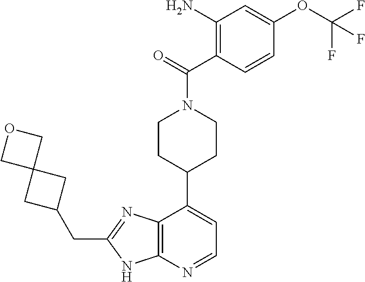 [SMILES: Nc1cc(OC(F)(F)F)ccc1C(=O)N1CCC(c2ccnc3[nH]c(CC4CC5(COC5)C4)nc23)CC1]

Step 2 of Example 50 was prepared following a similar procedure to that of step 7 of Example 29 from [2-nitro-4-(trifluoromethoxy)phenyl]-[4-[2-(2-oxaspiro[3.3]heptan-6-ylmethyl)-3H-imidazo[4,5-b]pyridin-7-yl]-3,6-dihydro-2H-pyridin-1-yl]methanone (125 mg, 0.23 mmol) with ammonium formate (551 mg, 8.74 mmol), Pd/C 10% (20 mg), in MeOH (10 mL) to give 61 mg (51%) of [2-amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone as a white solid. LC/MS (m/z, M+H, Method 1): calc. 516, found 516.2; ¹H NMR (400 MHz, DMSO-d6, 100° C.) δ ppm 1.80-1.99 (m, 4H), 1.96-2.07 (m, 2H), 2.32-2.40 (m, 2H), 2.54-2.65 (m, 1H), 2.86 (d, J=7 Hz, 2H), 3.03-3.15 (m, 2H), 3.29-3.46 (m, 1H), 4.17 (br d, J=13 Hz, 2H), 4.44 (s, 2H), 4.55 (s, 2H), 5.34 (s, 2H), 6.48 (br dd, J=8, 1 Hz, 1H), 6.68 (br d, J=1 Hz, 1H), 6.98 (d, J=5 Hz, 1H), 7.15 (d, J=8 Hz, 1H), 8.13 (d, J=5 Hz, 1H), 11.68-12.87 (m, 1H)

### Example 51: [2-Amino-4-(trifluoromethoxy)phenyl]-[4-[2-(oxetan-3-ylmethyl)-3H-imidazo[4,5-b]pyridin-7-yl]-1-piperidyl]methanone

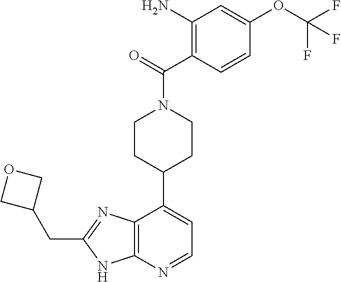 [SMILES: Nc1cc(OC(F)(F)F)ccc1C(=O)N1CCC(c2ccnc3[nH]c(CC4COC4)nc23)CC1]

### Step 1: [2-Nitro-4-(trifluoromethoxy)phenyl]-[4-[2-(oxetan-3-ylmethyl)-3H-imidazo[4,5-b]pyridin-7-yl]-3,6-dihydro-2H-pyridin-1-yl]methanone

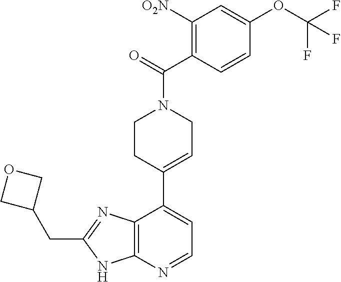 [SMILES: O=C(c1ccc(OC(F)(F)F)cc1[N+](=O)[O-])N1CC=C(c2ccnc3[nH]c(CC4COC4)nc23)CC1]

Step 1 of Example 51 was prepared following a similar procedure to that of step 1 of Example 20 from 2-(oxetan-3-yl)acetic acid (100 mg, 0.86 mmol) with [4-(2,3-diamino-4-pyridyl)-3,6-dihydro-2H-pyridin-1-yl]-[2-nitro-4-(trifluoromethoxy)phenyl]methanone (320 mg, 0.72 mmol), CDI (163 mg, 1.00 mmol), in DMF/pyridine (1.5 mL/1.5 mL) to give 129 mg (34%) of [2-nitro-4-(trifluoromethoxy)phenyl]-[4-[2-(oxetan-3-ylmethyl)-3H-imidazo[4,5-b]pyridin-7-yl]-3,6-dihydro-2H-pyridin-1-yl]methanone as a pale yellow solid. LC/MS (m/z, M+H): 504

### Step 2: [2-Amino-4-(trifluoromethoxy)phenyl]-[4-[2-(oxetan-3-ylmethyl)-3H-imidazo[4,5-b]pyridin-7-yl]-1-piperidyl]methanone

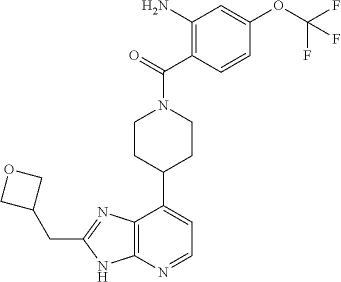 [SMILES: Nc1cc(OC(F)(F)F)ccc1C(=O)N1CCC(c2ccnc3[nH]c(CC4COC4)nc23)CC1]

Step 2 of Example 51 was prepared following a similar procedure to that of step 7 of Example 29 from [2-nitro-4-(trifluoromethoxy)phenyl]-[4-[2-(oxetan-3-ylmethyl)-3H-imidazo[4,5-b]pyridin-7-yl]-3,6-dihydro-2H-pyridin-1-yl]methanone (125 mg, 0.25 mmol) with ammonium formate (594 mg, 9.43 mmol), Pd/C 10% (20 mg), in MeOH (30 mL) to give 39 mg (33%) of [2-amino-4-(trifluoromethoxy)phenyl]-[4-[2-(oxetan-3-ylmethyl)-3H-imidazo[4,5-b]pyridin-7-yl]-1-piperidyl]methanone as a white solid. LC/MS (m/z, M+H, Method 1): calc. 476, found 476.2; ¹H NMR (400 MHz, DMSO-d6, 100° C.) δ ppm 1.82-1.98 (m, 4H), 3.04-3.16 (m, 2H), 3.20 (d, J=8 Hz, 2H), 3.28-3.41 (m, 1H), 3.44-3.59 (m, 1H), 4.12-4.22 (m, 2H), 4.43 (t, J=6 Hz, 2H), 4.72 (dd, J=8, 6 Hz, 2H), 5.34 (s, 2H), 6.48 (br dd, J=8, 1 Hz, 1H), 6.68 (br d, J=1 Hz, 1H), 6.99 (d, J=5 Hz, 1H), 7.14 (d, J=8 Hz, 1H), 8.14 (br d, J=5 Hz, 1H), 11.92-12.96 (m, 1H)

### Example 52: (4-Methoxy-1H-indol-7-yl)-[4-(2-tetrahydropyran-4-yl-3H-imidazo[4,5-b]pyridin-7-yl)-1-piperidyl]methanone

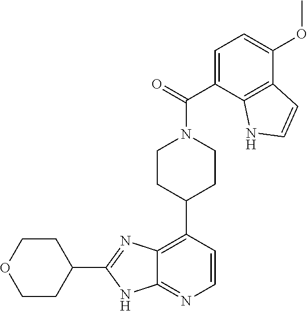 [SMILES: COc1ccc(C(=O)N2CCC(c3ccnc4[nH]c(C5CCOCC5)nc34)CC2)c2[nH]ccc12]

Example 52 was prepared following a similar procedure to that of step 5 of Examples 1 and 2 from 7-(4-piperidyl)-2-tetrahydropyran-4-yl-3H-imidazo[4,5-b]pyridine, hydrochloride (50 mg, 0.14 mmol) and 4-methoxy-indole-7-carboxylic acid (28 mg, 0.15 mmol) with TATU (52 mg, 0.16 mmol) and DIPEA (0.12 mL, 0.69 mmol) in DMF (1 mL) to give 45 mg (70%) of (4-methoxy-1H-indol-7-yl)-[4-(2-tetrahydropyran-4-yl-3H-imidazo[4,5-b]pyridin-7-yl)-1-piperidyl]methanone as a yellow solid. LC/MS (m/z, M+H, Method 1): calc. 460, found 460.2; ¹H NMR (400 MHz, DMSO-d6, 30° C., mixture of conformer 70/30) δ ppm 1.68-2.01 (m, 8H), 3.04-3.23 (m, 3H), 3.41-3.59 (m, 3H), 3.91 (s, 3H), 3.91-4.03 (m, 2H), 4.22-4.39 (m, 2H), 6.45-6.50 (m, 1H), 6.56 (d, J=8 Hz, 1H), 7.01 ((d, J=5 Hz, 1H), 7.12 ((d, J=8 Hz, 1H), 7.20 (m, 1H), 8.16 (d, J=5 Hz, 0.7H), 8.25 (br d, J=5 Hz, 0.3H), 11.03 (br s, 1H), 12.37 (s, 0.3H), 12.76 (s, 0.7H)

### Example 53: 6-[4-(2-Tetrahydropyran-4-yl-3H-imidazo[4,5-b]pyridin-7-yl)piperidine-1-carbonyl]-3-(trifluoromethyl)-1H-pyridin-2-one, hydrochloride

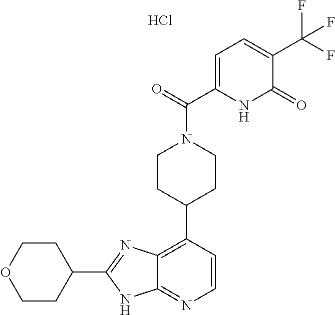 [SMILES: O=C(c1ccc(C(F)(F)F)c(=O)[nH]1)N1CCC(c2ccnc3[nH]c(C4CCOCC4)nc23)CC1 ; Cl]

### Step 1: 6-[4-(2-Tetrahydropyran-4-yl-3H-imidazo[4,5-b]pyridin-7-yl)piperidine-1-carbonyl]-3-(trifluoromethyl)-1H-pyridin-2-one

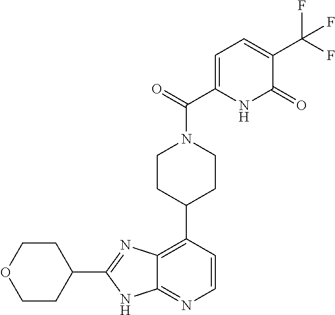 [SMILES: O=C(c1ccc(C(F)(F)F)c(=O)[nH]1)N1CCC(c2ccnc3[nH]c(C4CCOCC4)nc23)CC1]

Step 1 of Example 53 was prepared following a similar procedure to that of step 5 of Examples 1 and 2 from 7-(4-piperidyl)-2-tetrahydropyran-4-yl-3H-imidazo[4,5-b]pyridine, hydrochloride (150 mg, 0.42 mmol) and 6-oxo-5-(trifluoromethyl)-1,6-dihydropyridine-2-carboxylic acid (91 mg, 0.44 mmol) with HATU (175 mg, 0.46 mmol) and DIPEA (0.36 mL, 2.09 mmol) in DMF (2.5 mL) to give 66 mg (33%) of 6-[4-(2-tetrahydropyran-4-yl-3H-imidazo[4,5-b]pyridin-7-yl)piperidine-1-carbonyl]-3-(trifluoromethyl)-1H-pyridin-2-one as a pale brown foam. LC/MS (m/z, M+H): 476

### Step 2: 6-[4-(2-Tetrahydropyran-4-yl-3H-imidazo[4,5-b]pyridin-7-yl)piperidine-1-carbonyl]-3-(trifluoromethyl)-1H-pyridin-2-one, hydrochloride

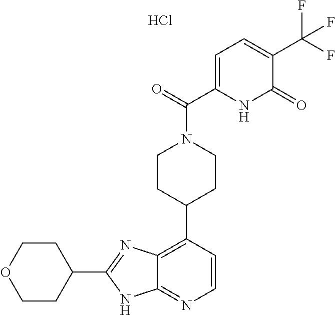 [SMILES: O=C(c1ccc(C(F)(F)F)c(=O)[nH]1)N1CCC(c2ccnc3[nH]c(C4CCOCC4)nc23)CC1 ; Cl]

6-[4-(2-Tetrahydropyran-4-yl-3H-imidazo[4,5-b]pyridin-7-yl)piperidine-1-carbonyl]-3-(trifluoromethyl)-1H-pyridin-2-one (66 mg, 0.14 mmol) was purified by preparative chromatography (reverse phase) with a C18 column 5 μm (250×50 mm), eluting with (water+0.1% HCl) and acetonitrile 100/0 to 0/100 (flow rate 80 mL/min, UV detection at 254 nm). Pure fractions were collected and concentrated, frozen and lyophilized to furnish 29 mg (38%) of 6-[4-(2-tetrahydropyran-4-yl-3H-imidazo[4,5-b]pyridin-7-yl)piperidine-1-carbonyl]-3-(trifluoromethyl)-1H-pyridin-2-one, hydrochloride as a white solid. LC/MS (m/z, M+H, Method 1): calc. 476 (free base), found 476.2; ¹H NMR (400 MHz, DMSO-d6, 100° C.) δ ppm 1.74-2.06 (m, 8H), 2.43-2.59 (m hidden, 1H), 2.97-3.22 (m, 2H), 3.24-3.38 (m, 1H), 3.41-3.55 (m, 2H), 3.55-3.71 (m, 1H), 3.97 (dt, J=11, 3 Hz, 2H), 6.49 (br d, J=6 Hz, 1H), 7.23 (d, J=5 Hz, 1H), 7.93 (d, J=7 Hz, 1H), 8.33 (d, J=5 Hz, 1H)

### Example 54: [4-(2-Tetrahydropyran-4-yl-3H-imidazo[4,5-b]pyridin-7-yl)-1-piperidyl]-[4-(2,2,2-trifluoroethyl)phenyl]methanone

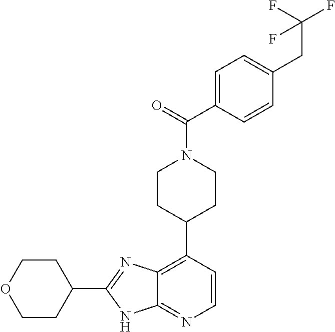 [SMILES: O=C(c1ccc(CC(F)(F)F)cc1)N1CCC(c2ccnc3[nH]c(C4CCOCC4)nc23)CC1]

Example 54 was prepared following a similar procedure to that of step 5 of Examples 1 and 2 from 7-(4-piperidyl)-2-tetrahydropyran-4-yl-3H-imidazo[4,5-b]pyridine, hydrochloride (100 mg, 0.28 mmol) and 4-(2,2,2-trifluoroethyl)benzoic acid (60 mg, 0.29 mmol) with TATU (103 mg, 0.32 mmol) and DIPEA (0.24 mL, 1.39 mmol) in DMF (2 mL) to give 62 mg (47%) of [4-(2-tetrahydropyran-4-yl-3H-imidazo[4,5-b]pyridin-7-yl)-1-piperidyl]-[4-(2,2,2-trifluoroethyl)phenyl]methanone as a white solid. LC/MS (m/z, M+H, Method 1): calc. 473, found 473.2; ¹H NMR (400 MHz, DMSO-d6, 100° C., mixture of conformer 70/30) δ ppm 1.67-2.06 (m, 8H), 3.03-3.21 (m, 3H), 3.23-3.56 (m, 3H), 3.65 (q, J=12 Hz, 2H), 3.95 (br d, J=12 Hz, 2H), 4.09-4.31 (m, 2H), 7.02 (d, J=5 Hz, 1H), 7.36-7.51 (m, 4H), 8.13 (br d, J=5 Hz, 0.7H), 8.18-8.29 (m, 0.3H), 12.00 (br s, 0.3H), 12.40 (br s, 0.70H)

### Example 55: [4-(Trifluoromethoxy)phenyl]-[4-[2-(trifluoromethyl)-3H-imidazo[4,5-b]pyridin-7-yl]-1-piperidyl]methanone

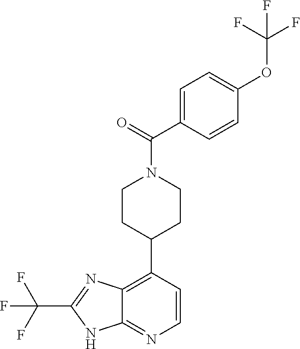 [SMILES: O=C(c1ccc(OC(F)(F)F)cc1)N1CCC(c2ccnc3[nH]c(C(F)(F)F)nc23)CC1]

### Step 1: 4-(4-Piperidyl)pyridine-2,3-diamine, 2,2,2-trifluoroacetic acid

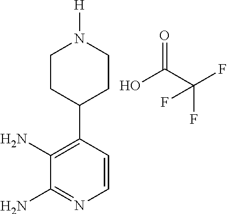

To a solution of tert-butyl 4-(2,3-diamino-4-pyridyl)piperidine-1-carboxylate (8 g, 27.4 mmol) in DCM (36 mL) was added TFA (16.9 mL, 218.9 mmol) and the resulting mixture was then stirred for 1 hour at room temperature. The reaction mixture was then concentrated to dryness to give 9.3 g of crude 4-(4-piperidyl)pyridine-2,3-diamine;2,2,2-trifluoroacetic acid as a brown wax which was engaged in the next step without further purification.

### Step 2: [4-(Trifluoromethoxy)phenyl]-[4-[2-(trifluoromethyl)-3H-imidazo[4,5-b]pyridin-7-yl]-1-piperidyl]methanone

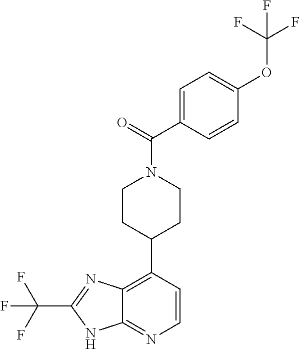 [SMILES: O=C(c1ccc(OC(F)(F)F)cc1)N1CCC(c2ccnc3[nH]c(C(F)(F)F)nc23)CC1]

To a solution of 4-(4-piperidyl)pyridine-2,3-diamine;2,2,2-trifluoroacetic acid (11.2 g, 36.6 mmol) in DMF (300 mL) and 4-(trifluoromethoxy)benzoic acid (9.04 g, 43.9 mmol) was added TBTU (17.6 g, 54.9 mmol) followed by triethylamine (25.5 mL, 183 mmol). The resulting mixture was then stirred for 1 hour at room temperature. Then, to the reaction mixture was added water (500 mL) and it was extracted three times with AcOEt (3×300 mL). The combined organic layers were then washed with water, dried over magnesium sulfate, filtered and concentrated in vacuo. The resulting residue was purified by flash chromatography (SiO₂ 300 g, eluting with DCM 98/MeOH 2) to give 2.58 g (18%) of [4-(trifluoromethoxy)phenyl]-[4-[2-(trifluoromethyl)-3H-imidazo[4,5-b]pyridin-7-yl]-1-piperidyl]methanone as a pale brown solid. LC/MS (m/z, M−H, Method 2): calc. 457, found 456.9; ¹H NMR (400 MHz, DMSO-d6, 100° C.) δ ppm 1.82-2.04 (m, 4H), 3.08-3.24 (m, 2H), 3.57 (tt, J=12, 4 Hz, 1H), 4.06-4.33 (m, 2H), 7.29 (d, J=5 Hz, 1H), 7.35-7.45 (m, 2H), 7.53-7.62 (m, 2H), 8.42 (d, J=5 Hz, 1H), 13.49-15.01 (m, 1H)

### Example 56: (Rac)-[4-(2-tetrahydropyran-2-yl-3H-imidazo[4,5-b]pyridin-7-yl)-1-piperidyl]-[4-(trifluoromethoxy)phenyl]methanone

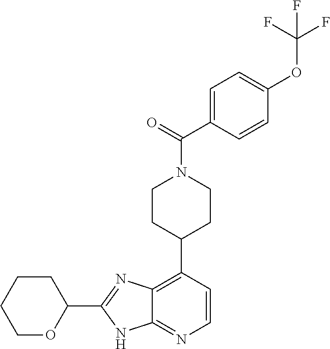 [SMILES: O=C(c1ccc(OC(F)(F)F)cc1)N1CCC(c2ccnc3[nH]c(C4CCCCO4)nc23)CC1]

### Step 1: (Rac)-tert-butyl 4-(2-tetrahydropyran-2-yl-3H-imidazo[4,5-b]pyridin-7-yl)piperidine-1-carboxylate

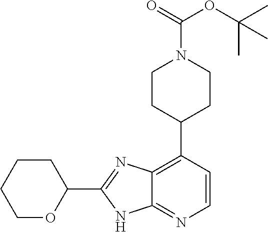 [SMILES: CC(C)(C)OC(=O)N1CCC(c2ccnc3[nH]c(C4CCCCO4)nc23)CC1]

Step 1 of Example 56 was prepared following a similar procedure to that of step 1 of Example 5 from tert-butyl 4-(2,3-diamino-4-pyridyl)piperidine-1-carboxylate (256 mg, 0.87 mmol) and (rac)-tetrahydropyran-2-carboxylic acid (171 mg, 1.31 mmol) with CDI (241 mg, 1.49 mmol) in acetonitrile (8 mL) under reflux for 36 hours to give 179 mg (53%) of (rac)-tert-butyl 4-(2-tetrahydropyran-2-yl-3H-imidazo[4,5-b]pyridin-7-yl)piperidine-1-carboxylate as a yellow solid. LC/MS (m/z, M+H): 387

### Step 2: (Rac)-7-(4-piperidyl)-2-tetrahydropyran-2-yl-3H-imidazo[4,5-b]pyridine, hydrochloride

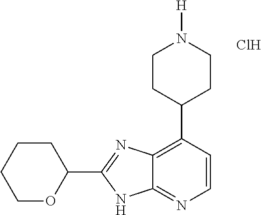 [SMILES: c1cc(C2CCNCC2)c2nc(C3CCCCO3)[nH]c2n1 ; Cl]

Step 2 of Example 56 was prepared following a similar procedure to that of step 4 of Examples 1 and 2 from (rac)-tert-butyl 4-(2-tetrahydropyran-2-yl-3H-imidazo[4,5-b]pyridin-7-yl)piperidine-1-carboxylate (179 mg, 0.46 mmol) using HCl 4N solution in 1,4-dioxane (1.16 mL, 4.63 mmol) in MeOH (7 mL) to give 179 mg (quantitative) of (rac)-7-(4-piperidyl)-2-tetrahydropyran-2-yl-3H-imidazo[4,5-b]pyridine, hydrochloride as a pale pink powder. LC/MS (m/z, M+H): 287 (free base)

### Step 3: (Rac)-[4-(2-tetrahydropyran-2-yl-3H-imidazo[4,5-b]pyridin-7-yl)-1-piperidyl]-[4-(trifluoromethoxy)phenyl]methanone

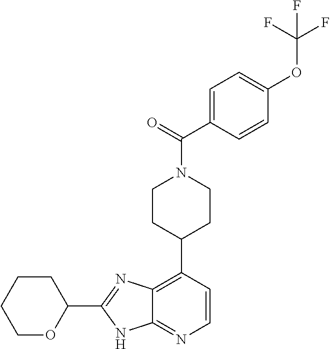 [SMILES: O=C(c1ccc(OC(F)(F)F)cc1)N1CCC(c2ccnc3[nH]c(C4CCCCO4)nc23)CC1]

Step 3 of Example 56 was prepared following a similar procedure to that of step 5 of Examples 1 and 2 from (rac)-7-(4-piperidyl)-2-tetrahydropyran-2-yl-3H-imidazo[4,5-b]pyridine, hydrochloride (89 mg, 0.28 mmol) and 4-(trifluoromethoxy)benzoic acid (60 mg, 0.29 mmol) with HATU (120 mg, 0.30 mmol) and DIPEA (0.28 mL, 1.7 mmol) in DMF (6 mL) to give 60 mg (50%) of (rac)-[4-(2-tetrahydropyran-2-yl-3H-imidazo[4,5-b]pyridin-7-yl)-1-piperidyl]-[4-(trifluoromethoxy)phenyl]methanone as a white solid. LC/MS (m/z, M+H, Method 1): calc. 475, found 475.2; ¹H NMR (400 MHz, DMSO-d6, 100° C.) δ ppm 1.53-2.13 (m, 10H), 3.05-3.18 (m, 2H), 3.42-3.54 (m, 1H), 3.58-3.69 (m, 1H), 4.02 (br d, J=11 Hz, 1H), 4.07-4.32 (m, 2H), 4.60-4.70 (m, 1H), 7.05 (d, J=5 Hz, 1H), 7.35-7.44 (m, 2H), 7.53-7.61 (m, 2H), 8.10-8.35 (m, 1H), 12.10-12.90 (m, 1H)

### Example 57: (Rac)-[2-amino-4-(trifluoromethoxy)phenyl]-[4-(2-tetrahydropyran-2-yl-3H-imidazo[4,5-b]pyridin-7-yl)-1-piperidyl]methanone

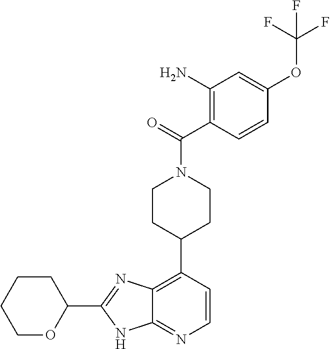 [SMILES: Nc1cc(OC(F)(F)F)ccc1C(=O)N1CCC(c2ccnc3[nH]c(C4CCCCO4)nc23)CC1]

Example 57 was prepared following a similar procedure to that of step 5 of Examples 1 and 2 from 7-(4-piperidyl)-2-tetrahydropyran-2-yl-3H-imidazo[4,5-b]pyridine, hydrochloride (89 mg, 0.28 mmol) and 2-amino-4-(trifluoromethoxy)benzoic acid (64 mg, 0.29 mmol) with HATU (120 mg, 0.30 mmol) and DIPEA (0.28 mL, 1.7 mmol) in DMF (2 mL) to give 62 mg (46%) of (rac)-[2-amino-4-(trifluoromethoxy)phenyl]-[4-(2-tetrahydropyran-2-yl-3H-imidazo[4,5-b]pyridin-7-yl)-1-piperidyl]methanone as a white solid. LC/MS (m/z, M+H, Method 1): calc. 490, found 490.2; ¹H NMR (400 MHz, DMSO-d6, 100° C.) δ ppm 1.52-2.10 (m, 10H), 3.02-3.17 (m, 2H), 3.46 (tt, J=11, 4 Hz, 1H), 3.55-3.68 (m, 1H), 3.97-4.12 (m, 1H), 4.16 (br d, J=13 Hz, 2H), 4.65 (dd, J=10, 3 Hz, 1H), 5.34 (s, 2H), 6.44-6.50 (m, 1H), 6.66-6.72 (m, 1H), 7.03 (d, J=5 Hz, 1H), 7.15 (d, J=8 Hz, 1H), 8.19 (d, J=5 Hz, 1H), 11.93-12.78 (m, 1H)

### Example 58: (3-Methoxy-1-bicyclo[1.1.1]pentanyl)-[4-(2-tetrahydropyran-4-yl-3H-imidazo[4,5-b]pyridin-7-yl)-1-piperidyl]methanone

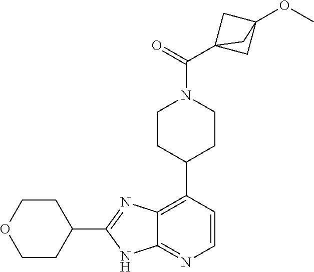 [SMILES: COC12CC(C(=O)N3CCC(c4ccnc5[nH]c(C6CCOCC6)nc45)CC3)(C1)C2]

Example 58 was prepared following a similar procedure to that of step 5 of Examples 1 and 2 from 7-(4-piperidyl)-2-tetrahydropyran-4-yl-3H-imidazo[4,5-b]pyridine, hydrochloride (150 mg, 0.42 mmol) and 3-methoxybicyclo[1.1.1]pentane-1-carboxylic acid (62 mg, 0.44 mmol) with HATU (182 mg, 0.48 mmol) and DIPEA (0.36 mL, 2.09 mmol) in DMF (2.5 mL) to give 92 mg (54%) of (3-methoxy-1-bicyclo[1.1.1]pentanyl)-[4-(2-tetrahydropyran-4-yl-3H-imidazo[4,5-b]pyridin-7-yl)-1-piperidyl]methanone as a white solid. LC/MS (m/z, M+H, Method 1): calc. 411, found 411.2; ¹H NMR (400 MHz, DMSO-d6, 30° C.) δ ppm 1.55-2.00 (m, 8H), 2.10-2.25 (m, 6H), 2.72 (br t, J=12 Hz, 1H), 3.06-3.26 (m, 2H), 3.21 (s, 3H), 3.34-3.56 (m, 3H), 3.91-4.03 (m, 2H), 4.17 (br d, J=14 Hz, 1H), 4.52 (br d, J=13 Hz, 1H), 7.02 (d, J=5 Hz, 1H), 8.10-8.25 (m, 1H), 12.25-13.00 (m, 1H)

### Example 59: [4-(2-Tetrahydropyran-4-yl-3H-imidazo[4,5-b]pyridin-7-yl)-1-piperidyl]-[3-(trifluoromethyl)-1-bicyclo[1.1.1]pentanyl]methanone

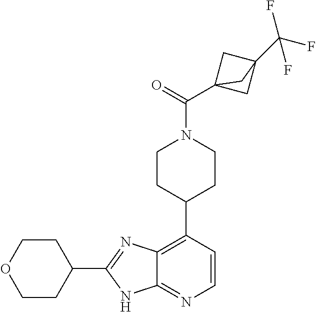 [SMILES: O=C(N1CCC(c2ccnc3[nH]c(C4CCOCC4)nc23)CC1)C12CC(C(F)(F)F)(C1)C2]

Example 59 was prepared following a similar procedure to that of step 5 of Examples 1 and 2 from 7-(4-piperidyl)-2-tetrahydropyran-4-yl-3H-imidazo[4,5-b]pyridine, hydrochloride (125 mg, 0.28 mmol) and 3-(trifluoromethyl)bicyclo[1.1.1]pentane-1-carboxylic acid (79 mg, 0.44 mmol) with HATU (182 mg, 0.48 mmol) and DIPEA (0.36 mL, 2.09 mmol) in DMF (2.5 mL) to give 125 mg (67%) of [4-(2-tetrahydropyran-4-yl-3H-imidazo[4,5-b]pyridin-7-yl)-1-piperidyl]-[3-(trifluoromethyl)-1-bicyclo[1.1.1]pentanyl]methanone as a white solid. LC/MS (m/z, M+H, Method 1): calc. 449, found 449.2; ¹H NMR (400 MHz, DMSO-d6, 30° C., mixture of conformer 75/25) δ ppm 1.50-2.05 (m, 8H), 2.25-2.41 (m, 6H), 2.69-2.83 (m, 1H), 3.08-3.31 (m partially hidden, 2H), 3.37-3.56 (m, 3H), 3.88-4.01 (m, 2H), 4.13-4.29 (m, 1H), 4.41-4.59 (m, 1H), 7.02 (d, J=5 Hz, 1H), 8.14 (d, J=5 Hz, 0.75H), 8.23 (d, J=5 Hz, 0.25H), 12.38 (s, 0.25H), 12.76 (s, 0.75H)

### Example 60: [4-(2-Tetrahydropyran-4-yl-3H-imidazo[4,5-b]pyridin-7-yl)-1-piperidyl]-[4-[1-(trifluoromethyl)cyclopropyl]phenyl]methanone

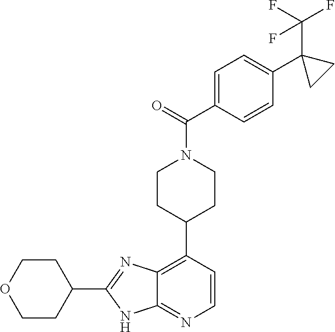 [SMILES: O=C(c1ccc(C2(C(F)(F)F)CC2)cc1)N1CCC(c2ccnc3[nH]c(C4CCOCC4)nc23)CC1]

Example 60 was prepared following a similar procedure to that of step 5 of Examples 1 and 2 from 7-(4-piperidyl)-2-tetrahydropyran-4-yl-3H-imidazo[4,5-b]pyridine, hydrochloride (150 mg, 0.42 mmol) and 4-(1-(trifluoromethyl)cyclopropyl)benzoic acid (101 mg, 0.44 mmol) with HATU (182 mg, 0.48 mmol) and DIPEA (0.36 mL, 2.09 mmol) in DMF (2.5 mL) to give 160 mg (77%) of [4-(2-tetrahydropyran-4-yl-3H-imidazo[4,5-b]pyridin-7-yl)-1-piperidyl]-[4-[1-(trifluoromethyl)cyclopropyl]phenyl]methanone as a pale brown solid. LC/MS (m/z, M+H, Method 1): calc. 499, found 499.1; ¹H NMR (400 MHz, DMSO-d6, 30° C., mixture of conformer 70/30) δ ppm 1.12-1.20 (m, 2H), 1.34-1.40 (m, 2H), 1.73-2.10 (m, 8H), 2.79-3.05 (m, 1H), 3.07-3.39 (m partially hidden, 3H), 3.48 (br t, J=11 Hz, 2H), 3.58-3.84 (m, 1H), 3.96 (br d, J=11 Hz, 2H), 4.53-4.83 (m, 1H), 7.09 (d, J=5 Hz, 1H), 7.47 (br d, J=8 Hz, 2H), 7.54 (br d, J=8 Hz, 2H), 8.17 (br s, 1H), 12.40 (br s, 0.3H), 12.77 (br s, 0.7H)

### Example 61: (Rac)-[4-[2-(5,5-difluoro-3-piperidyl)-3H-imidazo[4,5-b]pyridin-7-yl]-1-piperidyl]-[4-(trifluoromethoxy)phenyl]methanone

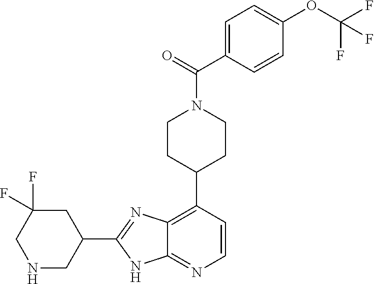 [SMILES: O=C(c1ccc(OC(F)(F)F)cc1)N1CCC(c2ccnc3[nH]c(C4CNCC(F)(F)C4)nc23)CC1]

### Step 1: (Rac)-tert-butyl 3,3-difluoro-5-[7-[1-[4-(trifluoromethoxy)benzoyl]-4-piperidyl]-3H-imidazo[4,5-b]pyridin-2-yl]piperidine-1-carboxylate

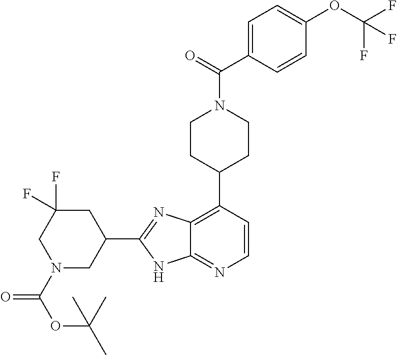 [SMILES: CC(C)(C)OC(=O)N1CC(c2nc3c(C4CCN(C(=O)c5ccc(OC(F)(F)F)cc5)CC4)ccnc3[nH]2)CC(F)(F)C1]

Step 1 of Example 61 was prepared following a similar procedure to that of step 1 of Example 5 from [4-(2,3-diamino-4-pyridyl)-1-piperidyl]-[4-(trifluoromethoxy)phenyl]methanone (100 mg, 0.26 mmol) and (rac)-1-(tert-butoxycarbonyl)-5,5-difluoropiperidine-3-carboxylic acid (240 mg, 0.90 mmol) with CDI (150 mg, 0.92 mmol) in acetonitrile (3 mL) under reflux for 96 hours to give 114 mg (71%) of (rac)-tert-butyl 3,3-difluoro-5-[7-[1-[4-(trifluoromethoxy)benzoyl]-4-piperidyl]-3H-imidazo[4,5-b]pyridin-2-yl]piperidine-1-carboxylate as a yellow solid. LC/MS (m/z, M+H): 610

### Step 2: (Rac)-[4-[2-(5,5-difluoro-3-piperidyl)-3H-imidazo[4,5-b]pyridin-7-yl]-1-piperidyl]-[4-(trifluoromethoxy)phenyl]methanone

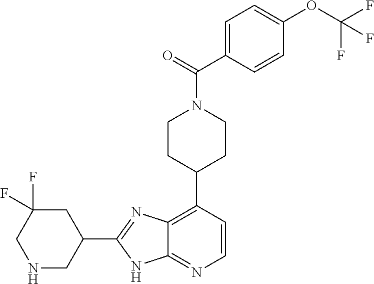 [SMILES: O=C(c1ccc(OC(F)(F)F)cc1)N1CCC(c2ccnc3[nH]c(C4CNCC(F)(F)C4)nc23)CC1]

Step 2 of Example 61 was prepared following a similar procedure to that of step 3 of Example 36 from (rac)-tert-butyl 3,3-difluoro-5-[7-[1-[4-(trifluoromethoxy)benzoyl]-4-piperidyl]-3H-imidazo[4,5-b]pyridin-2-yl]piperidine-1-carboxylate (110 mg, 0.18 mmol) with TFA (240 μL, 3.12 mmol) in DCM (3 mL) at room temperature for 16 hours to give 59 mg (64%) of (rac)-[4-[2-(5,5-difluoro-3-piperidyl)-3H-imidazo[4,5-b]pyridin-7-yl]-1-piperidyl]-[4-(trifluoromethoxy)phenyl]methanone as a yellow solid. LC/MS (m/z, M+H, Method 1): calc. 510, found 510.2; ¹H NMR (400 MHz, DMSO-d6, 120° C.) δ ppm 1.80-2.05 (m, 4H), 2.22-2.60 (m partially hidden, 2H), 2.73-2.97 (m partially hidden, 2H), 3.04-3.20 (m, 3H), 3.23-3.34 (m, 2H), 3.37-3.51 (m, 1H), 4.17 (br d, J=10 Hz, 2H), 7.03 (d, J=5 Hz, 1H), 7.37 (d, J=8 Hz, 2H), 7.56 (d, J=9 Hz, 2H), 8.12-8.22 (m, 1H), 11.75-12.95 (m, 1H)

### Example 62: [2-Amino-4-(trifluoromethoxy)phenyl]-[4-[2-(tetrahydropyran-4-ylmethyl)-3H-imidazo[4,5-b]pyridin-7-yl]-1-piperidyl]methanone

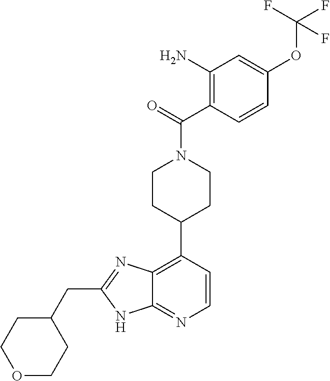 [SMILES: Nc1cc(OC(F)(F)F)ccc1C(=O)N1CCC(c2ccnc3[nH]c(CC4CCOCC4)nc23)CC1]

### Step 1: Tert-butyl 4-[2-(tetrahydropyran-4-ylmethyl)-3H-imidazo[4,5-b]pyridin-7-yl]piperidine-1-carboxylate

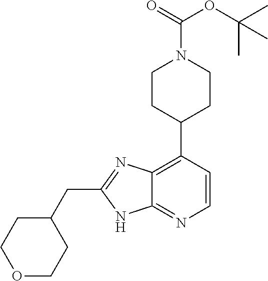 [SMILES: CC(C)(C)OC(=O)N1CCC(c2ccnc3[nH]c(CC4CCOCC4)nc23)CC1]

Step 1 of Example 62 was prepared following a similar procedure to that of step 1 of Example 5 from tert-butyl 4-(2,3-diamino-4-pyridyl)piperidine-1-carboxylate (250 mg, 0.85 mmol) and tetrahydropyranyl-4-acetic acid (180 mg, 1.25 mmol) with CDI (220 mg, 1.36 mmol) in acetonitrile (8.5 mL) under reflux for 48 hours to give 333 mg (97%) of tert-butyl 4-[2-(tetrahydropyran-4-ylmethyl)-3H-imidazo[4,5-b]pyridin-7-yl]piperidine-1-carboxylate as a brown solid. LC/MS (m/z, M+H): 401

### Step 2: 7-(4-Piperidyl)-2-(tetrahydropyran-4-ylmethyl)-3H-imidazo[4,5-b]pyridine, hydrochloride

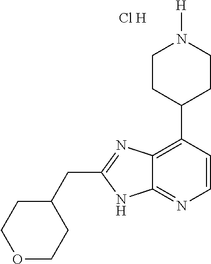 [SMILES: c1cc(C2CCNCC2)c2nc(CC3CCOCC3)[nH]c2n1 ; "Cl H"]

Step 2 of Example 62 was prepared following a similar procedure to that of step 4 of Examples 1 and 2 from tert-butyl 4-[2-(tetrahydropyran-4-ylmethyl)-3H-imidazo[4,5-b]pyridin-7-yl]piperidine-1-carboxylate (226 mg, 0.56 mmol) using HCl 4N solution in 1,4-dioxane (1.41 mL, 5.64 mmol) in MeOH (4 mL) to give 259 mg (quantitative) of 7-(4-piperidyl)-2-(tetrahydropyran-4-ylmethyl)-3H-imidazo[4,5-b]pyridine, hydrochloride as a brown wax. LC/MS (m/z, M+H): 301 (free base)

### Step 3: [2-Amino-4-(trifluoromethoxy)phenyl]-[4-[2-(tetrahydropyran-4-ylmethyl)-3H-imidazo[4,5-b]pyridin-7-yl]-1-piperidyl]methanone

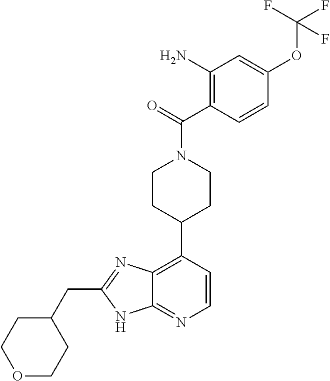 [SMILES: Nc1cc(OC(F)(F)F)ccc1C(=O)N1CCC(c2ccnc3[nH]c(CC4CCOCC4)nc23)CC1]

Step 3 of Example 62 was prepared following a similar procedure to that of step 5 of Examples 1 and 2 from 7-(4-piperidyl)-2-(tetrahydropyran-4-ylmethyl)-3H-imidazo[4,5-b]pyridine, hydrochloride (135 mg, 0.40 mmol) and 2-amino-4-(trifluoromethoxy)benzoic acid (93 mg, 0.42 mmol) with HATU (168 mg, 0.44 mmol) and DIPEA (0.34 mL, 2.0 mmol) in DMF (4 mL) to give 30 mg (10%) of [2-amino-4-(trifluoromethoxy)phenyl]-[4-[2-(tetrahydropyran-4-ylmethyl)-3H-imidazo[4,5-b]pyridin-7-yl]-1-piperidyl]methanone as a white solid. LC/MS (m/z, M+H, Method 1): calc. 504, found 504.2; ¹H NMR (400 MHz, DMSO-d6, 30° C.) δ ppm 1.30 (qd, J=12, 4 Hz, 2H), 1.56 (br d, J=12 Hz, 2H), 1.73-1.95 (m, 4H), 2.02-2.18 (m, 1H), 2.76 (d, J=7 Hz, 2H), 2.95-3.17 (m, 2H), 3.21-3.33 (m partially hidden, 2H), 3.34-3.55 (m, 1H), 3.82 (br dd, J=12, 3 Hz, 2H), 3.87-4.66 (m, 2H), 5.59 (br s, 2H), 6.49 (br d, J=8 Hz, 1H), 6.66 (br d, J=1 Hz, 1H), 7.04 (d, J=5 Hz, 1H), 7.15 (d, J=8 Hz, 1H), 8.06-8.31 (m, 1H), 12.25-13.25 (m, 1H)

### Example 63: (Rac)-[4-(2-pyrrolidin-3-yl-3H-imidazo[4,5-b]pyridin-7-yl)-1-piperidyl]-[4-(trifluoromethoxy)phenyl]methanone

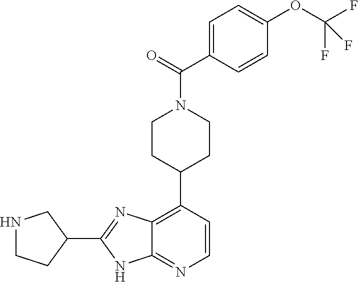 [SMILES: O=C(c1ccc(OC(F)(F)F)cc1)N1CCC(c2ccnc3[nH]c(C4CCNC4)nc23)CC1]

### Step 1: (Rac)-tert-butyl 4-[2-(1-benzyloxycarbonylpyrrolidin-3-yl)-3H-imidazo[4,5-b]pyridin-7-yl]piperidine-1-carboxylate

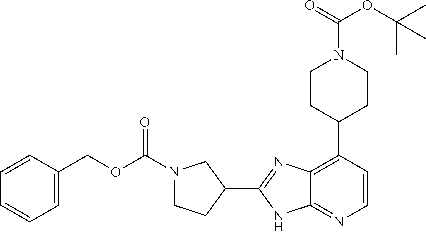 [SMILES: CC(C)(C)OC(=O)N1CCC(c2ccnc3[nH]c(C4CCN(C(=O)OCc5ccccc5)C4)nc23)CC1]

Step 1 of Example 63 was prepared following a similar procedure to that of step 1 of Example 20 from (rac)-1-benzyloxycarbonylpyrrolidine-3-carboxylic acid (307 mg, 1.23 mmol) with tert-butyl 4-(2,3-diamino-4-pyridyl)piperidine-1-carboxylate (300 mg, 1.03 mmol), CDI (233 mg, 1.43 mmol), in DMF/pyridine (1.5 mL/1.5 mL) to give 360 mg (69%) of (rac)-tert-butyl 4-[2-(1-benzyloxycarbonylpyrrolidin-3-yl)-3H-imidazo[4,5-b]pyridin-7-yl]piperidine-1-carboxylate as an orange solid. LC/MS (m/z, M+H): 506

### Step 2: (Rac)-benzyl 3-[7-(4-piperidyl)-3H-imidazo[4,5-b]pyridin-2-yl]pyrrolidine-1-carboxylate, hydrochloride

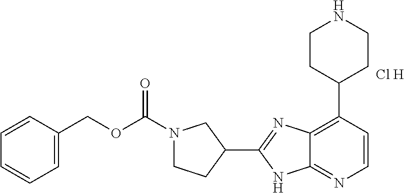

Step 2 of Example 63 was prepared following a similar procedure to that of step 4 of Examples 1 and 2 from (rac)-tert-butyl 4-[2-(1-benzyloxycarbonylpyrrolidin-3-yl)-3H-imidazo[4,5-b]pyridin-7-yl]piperidine-1-carboxylate (355 mg, 0.70 mmol) using HCl 4N solution in 1,4-dioxane (1.76 mL, 7.02 mmol) in DCM/MeOH (2 mL/4 mL) to give 336 mg (100%) of (rac)-benzyl 3-[7-(4-piperidyl)-3H-imidazo[4,5-b]pyridin-2-yl]pyrrolidine-1-carboxylate, hydrochloride as a brown powder. LC/MS (m/z, M+H): 406 (free base)

### Step 3: (Rac)-benzyl 3-[7-[1-[4-(trifluoromethoxy)benzoyl]-4-piperidyl]-3H-imidazo[4,5-b]pyridin-2-yl]pyrrolidine-1-carboxylate

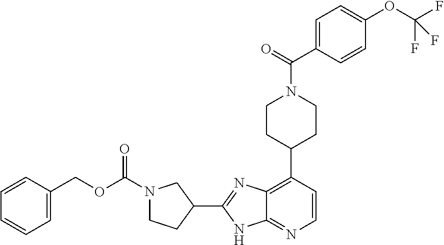 [SMILES: O=C(OCc1ccccc1)N1CCC(c2nc3c(C4CCN(C(=O)c5ccc(OC(F)(F)F)cc5)CC4)ccnc3[nH]2)C1]

Step 3 of Example 63 was prepared following a similar procedure to that of step 5 of Examples 1 and 2 from (rac)-benzyl 3-[7-(4-piperidyl)-3H-imidazo[4,5-b]pyridin-2-yl]pyrrolidine-1-carboxylate, hydrochloride (335 mg, 0.76 mmol) and 4-(trifluoromethoxy)benzoic acid (172 mg, 0.83 mmol) with HATU (346 mg, 0.91 mmol) and DIPEA (0.66 mL, 3.79 mmol) in DMF (5 mL) to give 348 mg (70%) of (rac)-benzyl 3-[7-[1-[4-(trifluoromethoxy)benzoyl]-4-piperidyl]-3H-imidazo[4,5-b]pyridin-2-yl]pyrrolidine-1-carboxylate as a yellow solid. LC/MS (m/z, M+H): 594

### Step 4: (Rac)-[4-(2-pyrrolidin-3-yl-3H-imidazo[4,5-b]pyridin-7-yl)-1-piperidyl]-[4-(trifluoromethoxy)phenyl]methanone

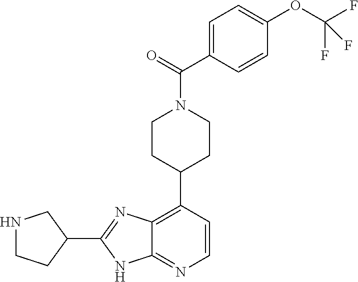 [SMILES: O=C(c1ccc(OC(F)(F)F)cc1)N1CCC(c2ccnc3[nH]c(C4CCNC4)nc23)CC1]

To a solution of (rac)-benzyl 3-[7-[1-[4-(trifluoromethoxy)benzoyl]-4-piperidyl]-3H-imidazo[4,5-b]pyridin-2-yl]pyrrolidine-1-carboxylate (345 mg, 0.53 mmol) in MeOH (8 mL) was added Pd/C 10% (35 mg). The resulting residue was then submitted to hydrogen on a Parr apparatus with 3 bars of H₂ at room temperature for 14 hours. After 14 hours, the reaction mixture was filtered, concentrated and the resulting residue was purified by flash chromatography (SiO₂ 4 g, eluting with DCM 80/MeOH 20/NH₄OH 2) to give 174 mg (72%) of (rac)-[4-(2-pyrrolidin-3-yl-3H-imidazo[4,5-b]pyridin-7-yl)-1-piperidyl]-[4-(trifluoromethoxy)phenyl]methanone as a pale brown solid. LC/MS (m/z, M+H, Method 1): calc. 460, found 460.2; ¹H NMR (400 MHz, DMSO-d6, 120° C.) δ ppm 1.82-2.00 (m, 4H), 2.04-2.24 (m, 2H), 2.92 (dt, J=11, 7 Hz, 1H), 3.00-3.18 (m, 4H), 3.23 (dd, J=11, 8 Hz, 1H), 3.36-3.50 (m, 2H), 4.11-4.24 (m, 2H), 7.00 (d, J=5 Hz, 1H), 7.37 (d, J=8 Hz, 2H), 7.56 (d, J=8 Hz, 2H), 8.13 (d, J=5 Hz, 1H)

### Example 64: [4-[2-[(1-Methyl-4-piperidyl)methyl]-3H-imidazo[4,5-b]pyridin-7-yl]-1-piperidyl]-[4-(trifluoromethoxy)phenyl]methanone

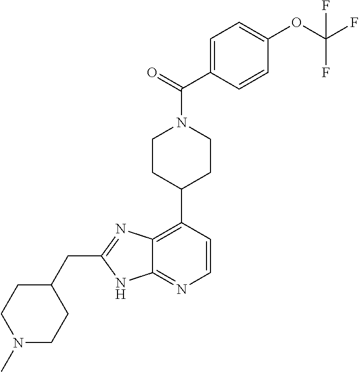 [SMILES: CN1CCC(Cc2nc3c(C4CCN(C(=O)c5ccc(OC(F)(F)F)cc5)CC4)ccnc3[nH]2)CC1]

To a solution of 2-(1-methyl-4-piperidyl)acetic acid, hydrochloride (78 mg, 0.39 mmol) in acetonitrile (5.1 mL) at room temperature under argon atmosphere was added DIEA (0.07 mL, 0.39 mmol). The reaction mixture was stirred for 5 minutes at room temperature and CDI (76 mg, 0.47 mmol) was added. The resulting mixture was then stirred one hour at room temperature. Then, [4-(2,3-diamino-4-pyridyl)-1-piperidyl]-[4-(trifluoromethoxy)phenyl]methanone (150 mg, 0.39 mmol) was added and the resulting mixture was stirred under reflux for 16 hours. Then, a pre-mixed solution 2-(1-methyl-4-piperidyl)acetic acid, hydrochloride (101 mg, 0.64 mmol), DIEA (90 μL, 0.50 mmol) and CDI (100 mg, 0.62 mmol) in acetonitrile (5 mL) was added to the reaction mixture which was then refluxed for 12 additional hours. After 12 hours, once again, a pre-mixed solution 2-(1-methyl-4-piperidyl)acetic acid, hydrochloride (101 mg, 0.64 mmol), DIEA (90 μL, 0.50 mmol) and CDI (100 mg, 0.62 mmol) in acetonitrile (5 mL) was added to the reaction mixture which was then refluxed for 12 additional hours. The mixture was cooled down to room temperature and concentrated to dryness. The resulting residue was diluted with AcOEt, transferred in a separating funnel, washed with a 1N aqueous solution of NaOH and with brine. The aqueous layer was extracted with AcOEt. The combined organic layers were then dried over sodium sulfate, filtered and concentrated. The resulting residue was then purified to give 77 mg (39%) of [4-[2-[(1-methyl-4-piperidyl)methyl]-3H-imidazo[4,5-b]pyridin-7-yl]-1-piperidyl]-[4-(trifluoromethoxy)phenyl]methanone as a white solid. LC/MS (m/z, M+H, Method 1): calc. 502, found 502.2; ¹H NMR (400 MHz, DMSO-d6, 120° C.) δ ppm 1.33 (dq, J=12, 4 Hz, 2H), 1.64 (br d, J=12 Hz, 2H), 1.78-2.01 (m, 7H), 2.14 (s, 3H), 2.66-2.73 (m, 2H), 2.75 (d, J=7 Hz, 2H), 3.07-3.20 (m, 2H), 3.35-3.47 (m, 1H), 4.12-4.23 (m, 2H), 6.98 (d, J=5 Hz, 1H), 7.36 (br d, J=8 Hz, 2H), 7.56 ((d, J=8 Hz, 2H), 8.13 (d, J=5 Hz, 1H), 11.85-12.62 (m, 1H)

### Example 65: (Rac)-[2-amino-4-(trifluoromethoxy)phenyl]-[4-(2-tetrahydropyran-3-yl-3H-imidazo[4,5-b]pyridin-7-yl)-1-piperidyl]methanone

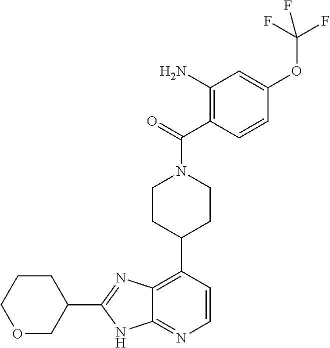 [SMILES: Nc1cc(OC(F)(F)F)ccc1C(=O)N1CCC(c2ccnc3[nH]c(C4CCCOC4)nc23)CC1]

### Step 1: (Rac)-tert-butyl 4-(2-tetrahydropyran-3-yl-3H-imidazo[4,5-b]pyridin-7-yl)piperidine-1-carboxylate

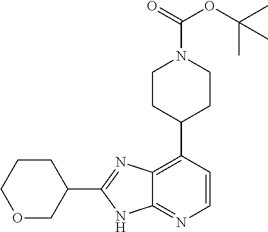 [SMILES: CC(C)(C)OC(=O)N1CCC(c2ccnc3[nH]c(C4CCCOC4)nc23)CC1]

Step 1 of Example 65 was prepared following a similar procedure to that of step 1 of Example 5 from tert-butyl 4-(2,3-diamino-4-pyridyl)piperidine-1-carboxylate (250 mg, 0.85 mmol) and tetrahydro-2H-pyran-3carboxylic acid (148 mg, 1.214 mmol) with CDI (220 mg, 1.36 mmol) in acetonitrile (8.5 mL) under reflux for 48 hours to give 192 mg (58%) of (rac)-tert-butyl 4-(2-tetrahydropyran-3-yl-3H-imidazo[4,5-b]pyridin-7-yl)piperidine-1-carboxylate as a pink solid. LC/MS (m/z, M+H): 387

### Step 2: (Rac)-7-(4-piperidyl)-2-tetrahydropyran-3-yl-3H-imidazo[4,5-b]pyridine, hydrochloride

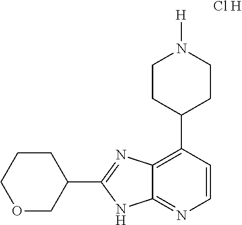

Step 2 of Example 65 was prepared following a similar procedure to that of step 4 of Examples 1 and 2 from (rac)-tert-butyl 4-(2-tetrahydropyran-3-yl-3H-imidazo[4,5-b]pyridin-7-yl)piperidine-1-carboxylate (100 mg, 0.26 mmol) using HCl 4N solution in 1,4-dioxane (0.65 mL, 2.59 mmol) in MeOH (2 mL) to give 83 mg (quantitative) of (rac)-7-(4-piperidyl)-2-tetrahydropyran-3-yl-3H-imidazo[4,5-b]pyridine, hydrochloride as a pale brown wax. LC/MS (m/z, M+H): 287 (free base)

### Step 3: (Rac)-[2-amino-4-(trifluoromethoxy)phenyl]-[4-(2-tetrahydropyran-3-yl-3H-imidazo[4,5-b]pyridin-7-yl)-1-piperidyl]methanone

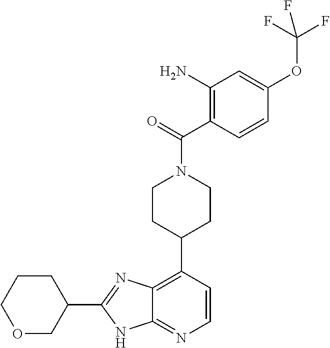 [SMILES: Nc1cc(OC(F)(F)F)ccc1C(=O)N1CCC(c2ccnc3[nH]c(C4CCCOC4)nc23)CC1]

Step 3 of Example 65 was prepared following a similar procedure to that of step 5 of Examples 1 and 2 from (rac)-7-(4-piperidyl)-2-tetrahydropyran-3-yl-3H-imidazo[4,5-b]pyridine, hydrochloride (89 mg, 0.28 mmol) and 2-amino-4-(trifluoromethoxy)benzoic acid (64 mg, 0.29 mmol) with HATU (120 mg, 0.30 mmol) and DIPEA (0.24 mL, 1.4 mmol) in DMF (3 mL) to give 67 mg (50%) of [(rac)-[2-amino-4-(trifluoromethoxy)phenyl]-[4-(2-tetrahydropyran-3-yl-3H-imidazo[4,5-b]pyridin-7-yl)-1-piperidyl]methanone as a white solid. LC/MS (m/z, M+H, Method 1): calc. 490, found 490.2; ¹H NMR (400 MHz, DMSO-d6, 30° C., mixture of conformer 70/30) δ ppm 1.57-2.02 (m, 8H), 2.10-2.21 (m, 1H), 2.96-3.19 (m, 2H), 3.22-3.35 (m partially hidden, 1H), 3.37-3.52 (m, 2H), 3.57-3.67 (m, 1H), 3.83-3.93 (m, 1H), 3.94-4.67 (m, 1H), 4.05-4.13 (m, 1H), 5.58 (s, 1.4H), 5.61 (s, 0.6H), 6.49 (br d, J=8 Hz, 1H), 6.65-6.70 (m, 1H), 7.03-7.10 (m, 1H), 7.13-7.20 (m, 1H), 8.16 (d, J=5 Hz, 0.7H), 8.24 (d, J=5 Hz, 0.3H), 12.43 (s, 0.3H), 12.78 (s, 0.7H)

### Example 66: [4-[2-(Tetrahydropyran-4-ylmethyl)-3H-imidazo[4,5-b]pyridin-7-yl]-1-piperidyl]-[4-(trifluoromethoxy)phenyl]methanone

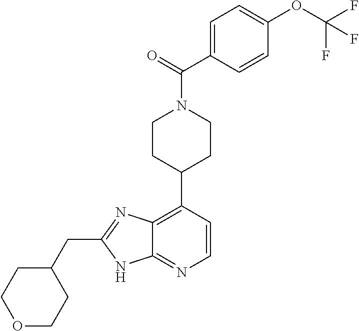 [SMILES: O=C(c1ccc(OC(F)(F)F)cc1)N1CCC(c2ccnc3[nH]c(CC4CCOCC4)nc23)CC1]

Example 66 was prepared following a similar procedure to that of step 5 of Examples 1 and 2 from 7-(4-piperidyl)-2-(tetrahydropyran-4-ylmethyl)-3H-imidazo[4,5-b]pyridine, hydrochloride (135 mg, 0.40 mmol) and 4-(trifluoromethoxy)benzoic acid (87 mg, 0.42 mmol) with HATU (168 mg, 0.4 mmol) and DIPEA (0.34 mL, 2 mmol) in DMF (3 mL) to give 40 mg (20%) of [4-[2-(tetrahydropyran-4-ylmethyl)-3H-imidazo[4,5-b]pyridin-7-yl]-1-piperidyl]-[4-(trifluoromethoxy)phenyl]methanone as a white solid. LC/MS (m/z, M+H, Method 1): calc. 489, found 489.2; ¹H NMR (400 MHz, DMSO-d6, 30° C.) δ ppm 1.30 (dq, J=12, 4 Hz, 2H), 1.56 (br d, J=12 Hz, 2H), 1.68-2.02 (m, 4H), 2.02-2.17 (m, 1H), 2.77 (d, J=7 Hz, 2H), 2.87-3.10 (m, 1H), 3.15-3.36 (m partially hidden, 3H), 3.37-3.54 (m, 1H), 3.57-3.75 (m, 1H), 3.78-3.88 (m, 2H), 4.56-4.80 (m, 1H), 7.06 (d, J=5 Hz, 1H), 7.45 (d, J=8 Hz, 2H), 7.60 (d, J=8 Hz, 2H), 8.08-8.25 (m, 1H), 12.13-13.00 (m, 1H)

### Example 67: (Rac)-[4-(2-tetrahydropyran-3-yl-3H-imidazo[4,5-b]pyridin-7-yl)-1-piperidyl]-[4-(trifluoromethoxy)phenyl]methanone

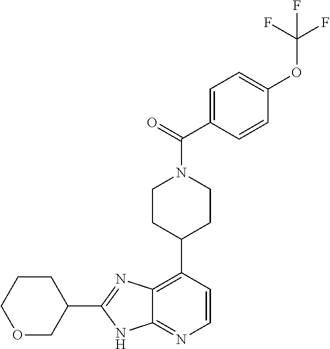 [SMILES: O=C(c1ccc(OC(F)(F)F)cc1)N1CCC(c2ccnc3[nH]c(C4CCCOC4)nc23)CC1]

Example 67 was prepared following a similar procedure to that of step 5 of Examples 1 and 2 from 7-(4-piperidyl)-2-tetrahydropyran-3-yl-3H-imidazo[4,5-b]pyridine, hydrochloride (74 mg, 0.23 mmol) and 4-(trifluoromethoxy)benzoic acid (50 mg, 0.24 mmol) with HATU (96 mg, 0.25 mmol) and DIPEA (0.20 mL, 1.1 mmol) in DMF (5 mL) to give 49 mg (45%) of (rac)-[4-(2-tetrahydropyran-3-yl-3H-imidazo[4,5-b]pyridin-7-yl)-1-piperidyl]-[4-(trifluoromethoxy)phenyl]methanone as a white solid. LC/MS (m/z, M+H, Method 1): calc. 475, found 475.2; ¹H NMR (400 MHz, DMSO-d6, 30° C.) δ ppm 1.53-2.07 (m, 7H), 2.15 (br d, J=13 Hz, 1H), 2.83-3.03 (m, 1H), 3.03-3.16 (m, 1H), 3.20-3.35 (m hidden, 1H), 3.37-3.55 (m, 1H), 3.42 (td, J=11, 3 Hz, 1H), 3.61 (t, J=11 Hz, 1H), 3.56-3.79 (m, 1H), 3.87 (br d, J=11 Hz, 1H), 4.08 (br dd, J=11, 3 Hz, 1H), 4.54-4.80 (m, 1H), 7.09 (d, J=5 Hz, 1H), 7.45 (dd, J=9, 1 Hz, 2H), 7.60 (d, J=9 Hz, 2H), 8.18 (m, 1H), 12.00-13.20 (m, 1H)

### Example 68: [4-[2-[1-(2,2-Difluoroethyl)-4-piperidyl]-3H-imidazo[4,5-b]pyridin-7-yl]-1-piperidyl]-[4-(trifluoromethoxy)phenyl]methanone

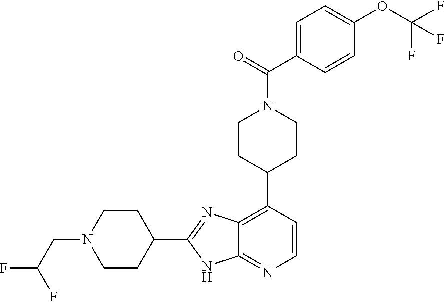 [SMILES: O=C(c1ccc(OC(F)(F)F)cc1)N1CCC(c2ccnc3[nH]c(C4CCN(CC(F)F)CC4)nc23)CC1]

In a microwave vial, to a solution of [4-(2,3-diamino-4-pyridyl)-1-piperidyl]-[4-(trifluoromethoxy)phenyl]methanone (100 mg, 0.26 mmol) and 1-(2,2-difluoroethyl)piperidine-4-carboxylic acid hydrochloride (60 mg, 0.26 mmol) in acetonitrile (600 μL) at room temperature was added N,N-diisopropylethylamine (225 μL, 1.29 mmol) followed by T3P (50% in AcOEt) (160 μL, 0.27 mmol). The vial was sealed and irradiated under microwave for 60 minutes at 150° C. 1-(2,2-Difluoroethyl)piperidine-4-carboxylic acid hydrochloride (60 mg, 0.26 mmol) and N,N-diisopropylethylamine (225 μL, 1.29 mmol) and T3P (50% in AcOEt) (160 μL, 0.27 mmol) were added. The vial was then submitted to microwave irradiation for 45 minutes at 150° C. The reaction mixture was purified by flash chromatography (SiO₂ 12 g, eluting with DCM 95/MeOH 5) to give 21 mg (15%) of [4-[2-[1-(2,2-difluoroethyl)-4-piperidyl]-3H-imidazo[4,5-b]pyridin-7-yl]-1-piperidyl]-[4-(trifluoromethoxy)phenyl]methanone as a white solid. LC/MS (m/z, M+H, Method 1): calc. 538, found 538.3; ¹H NMR (400 MHz, DMSO-d6, 30° C., mixture of conformer 70/30) δ ppm 1.65-2.06 (m, 8H), 2.25-2.39 (m, 2H), 2.76 (td, J=16, 4 Hz, 2H), 2.81-3.09 (m, 4H), 3.14-3.40 (m partially hidden, 1H), 3.41-3.54 (m, 1H), 3.57-3.84 (m, 1H), 4.50-4.90 (m, 1H), 6.15 (tt, J=56, 4 Hz, 1H), 7.07 (d, J=5 Hz, 1H), 7.45 (br d, J=8 Hz, 2H), 7.56-7.68 (m, 2H), 8.15 (d, J=5 Hz, 0.7H), 8.24 (d, J=5 Hz, 0.3H), 12.35 (s, 0.3H), 12.72 (s, 0.7H)

### Example 69: [2-Amino-4-(trifluoromethoxy)phenyl]-[4-[2-(oxetan-3-yl)-3H-imidazo[4,5-b]pyridin-7-yl]-1-piperidyl]methanone

Example 69 was prepared following a procedure similar to that of step 1 of Example 15, using [4-(2,3-diamino-4-pyridyl)-3,6-dihydro-2H-pyridin-1-yl]-[2-nitro-4-(trifluoromethoxy)phenyl]methanone and the corresponding carboxylic acid, followed by a procedure similar to step 7 of Example 29.

Examples 70 and 71: [4-[2-[Tetrahydrofuran-2-yl]-3H-imidazo[4,5-b]pyridin-7-yl]-1-piperidyl]-[4-(trifluoromethoxy)phenyl]methanone, Isomers 1 and 2

Examples 70 and 71 were obtained by chiral separation of the compound of Example 34 using Method CS1.

Example 70 (Isomer 1): LC/MS (m/z, M+H): 461; ¹H NMR (500 MHz, DMSO-d6, 25° C.) 70/30 mixture of conformers δ ppm 1.66-2.07 (m, 6H), 2.14-2.37 (m, 2H), 2.88-3.06 (m, 1H), 3.19-3.30 (partially hidden, m, 1H), 3.42-3.53 (m, 1H), 3.60-3.73 (m, 1H), 3.80-3.93 (m, 1H), 3.95-4.06 (m, 1H), 4.60-4.73 (m, 1H), 5.06 (t, J=6.9 Hz, 0.7H), 5.14 (t, J=6.9 Hz, 0.3H), 7.12 (br d, J=5.0 Hz, 1H), 7.46 (d, J=8.3 Hz, 2H), 7.57-7.66 (m, 2H), 8.20 (d, J=5.0 Hz, 0.7H), 8.27 (d, J=5.0 Hz, 0.3H), 12.69 (s, 0.3H), 12.93 (br s, 0.7H)

### Example 72: [4-[2-(3-hydroxy-1-bicyclo[1.1.1]pentanyl)-3H-imidazo[4,5-b]pyridin-7-yl]-1-piperidyl]-[4-(trifluoromethoxy)phenyl]methanone

Example 72 was prepared following a procedure similar to that of step 1 of Example 15. LC/MS (m/z, M+H): 473

Examples 73 and 74: 4-[2-[pyrrolidin-3-yl]-3H-imidazo[4,5-b]pyridin-7-yl]-1-piperidyl]-[4-(trifluoromethoxy)phenyl]methanone, Isomers 1 and 2

Examples 73 and 74 were obtained by chiral separation of the compound of Example 63 using Method CS2.

Example 73 (Isomer 1): LC/MS (m/z, M+H): 460

### Examples 75 and 76: [2-amino-4-(trifluoromethoxy)phenyl]-[4-[2-[tetrahydropyran-2-yl]-3H-imidazo[4,5-b]pyridin-7-yl]-1-piperidyl]methanone, Isomers 1 and 2

Examples 75 and 76 were obtained by chiral separation of the compound of Example 57 using Method CS3.

Example 75 (Isomer 1): LC/MS (m/z, M+H): 490; ¹H NMR (400 MHz, DMSO-d6, 100° C.) δ ppm 1.56-1.76 (m, 4H), 1.80-2.07 (m, 6H), 3.04-3.15 (m, 2H), 3.39-3.53 (m, 1H), 3.58-3.68 (m, 1H), 3.98-4.07 (m, 1H), 4.16 (br d, J=13.3 Hz, 2H), 4.61-4.69 (m, 1H), 5.33 (br s, 2H), 6.48 (br d, J=8.3 Hz, 1H), 6.66-6.71 (m, 1H), 7.03 (d, J=5.0 Hz, 1H), 7.15 (d, J=8.3 Hz, 1H), 8.19 (br d, J=5.0 Hz, 1H), 12.11-12.60 (m, 1H)

Example 76 (Isomer 2): LC/MS (m/z, M+H): 490

### Examples 77-89

Examples 77-89 were prepared following a procedure similar to that of step 5 of Examples 1 and 2.

Example 77: 2-naphthyl-[4-(2-tetrahydropyran-4-yl-3H-imidazo[4,5-b]pyridin-7-yl)-1-piperidyl]methanone; LC/MS (m/z, M+H): 441.2

Example 78: 1H-indazol-3-yl-[4-(2-tetrahydropyran-4-yl-3H-imidazo[4,5-b]pyridin-7-yl)-1-piperidyl]methanone; LC/MS (m/z, M+H): 431.2

Example 79: [4-(2-tetrahydropyran-4-yl-3H-imidazo[4,5-b]pyridin-7-yl)-1-piperidyl]-[7-(trifluoromethyl)-4,5,6,7-tetrahydropyrazolo[1,5-a]pyrimidin-3-yl]methanone; LC/MS (m/z, M+H): 504.2

Example 80: (1-methyl-5-phenyl-pyrazol-3-yl)-[4-(2-tetrahydropyran-4-yl-3H-imidazo[4,5-b]pyridin-7-yl)-1-piperidyl]methanone; LC/MS (m/z, M+H): 471.3

Example 81: (3-amino-2-naphthyl)-[4-(2-tetrahydropyran-4-yl-3H-imidazo[4,5-b]pyridin-7-yl)-1-piperidyl]methanone; LC/MS (m/z, M+H): 456.3

Example 82: (2-amino-3,4,5,6-tetrafluoro-phenyl)-[4-(2-tetrahydropyran-4-yl-3H-imidazo[4,5-b]pyridin-7-yl)-1-piperidyl]methanone; LC/MS (m/z, M+H): 478.2

Example 83: [1-isopropyl-2-(trifluoromethyl)benzimidazol-5-yl]-[4-(2-tetrahydropyran-4-yl-3H-imidazo[4,5-b]pyridin-7-yl)-1-piperidyl]methanone; LC/MS (m/z, M+H): 541.3

Example 84: (2-amino-4-methylsulfonyl-phenyl)-[4-(2-tetrahydropyran-4-yl-3H-imidazo[4,5-b]pyridin-7-yl)-1-piperidyl]methanone; LC/MS (m/z, M+H): 484.2

Example 85: [3-(1,1-dioxo-1,2-thiazolidin-2-yl)phenyl]-[4-(2-tetrahydropyran-4-yl-3H-imidazo[4,5-b]pyridin-7-yl)-1-piperidyl]methanone; LC/MS (m/z, M+H): 510.2

Example 86: [2-(3-hydroxyphenyl)cyclopropyl]-[4-(2-tetrahydropyran-4-yl-3H-imidazo[4,5-b]pyridin-7-yl)-1-piperidyl]methanone; LC/MS (m/z, M+H): 447.3

Example 87: 2-[2-chloro-4-(trifluoromethyl)phenoxy]-1-[4-(2-tetrahydropyran-4-yl-3H-imidazo[4,5-b]pyridin-7-yl)-1-piperidyl]propan-1-one; LC/MS (m/z, M+H): 537.2

Example 88: (3-phenylcyclopentyl)-[4-(2-tetrahydropyran-4-yl-3H-imidazo[4,5-b]pyridin-7-yl)-1-piperidyl]methanone; LC/MS (m/z, M+H): 459.3

Example 89: [4-hydroxy-1-[2-(trifluoromethyl)phenyl]pyrazol-3-yl]-[4-(2-tetrahydropyran-4-yl-3H-imidazo[4,5-b]pyridin-7-yl)-1-piperidyl]methanone; LC/MS (m/z, M+H): 541.2

### Examples 90 and 91: [4-[2-[5,5-difluoro-3-piperidyl]-3H-imidazo[4,5-b]pyridin-7-yl]-1-piperidyl]-[4-(trifluoromethoxy)phenyl]methanone, Isomers 1 and 2

Examples 90 and 91 were obtained by chiral separation of the compound of Example 61 using Method CS4.

Example 90 (Isomer 1): LC/MS (m/z, M+H): 510

Example 92 and 93 Examples 92 and 93 were prepared following a procedure similar to that of step 5 of Examples 1 and 2.

Example 92: [3-amino-4-phenyl-5-(trifluoromethyl)-2-thienyl]-[4-(2-tetrahydropyran-4-yl-3H-imidazo[4,5-b]pyridin-7-yl)-1-piperidyl]methanone; LC/MS (m/z, M+H): 556.2

Example 93: (7-hydroxy-1H-indol-2-yl)-[4-(2-tetrahydropyran-4-yl-3H-imidazo[4,5-b]pyridin-7-yl)-1-piperidyl]methanone; LC/MS (m/z, M+H): 446.2

### Examples 94 and 95

Examples 94 and 95 were prepared following a procedure similar to that of step 1 of Example 20 using [4-(2,3-diamino-4-pyridyl)-1-piperidyl]-[4-(trifluoromethoxy)phenyl]methanone and the corresponding carboxylic acid.

Example 94: [4-[2-(1-cyclopropyl-4-piperidyl)-3H-imidazo[4,5-b]pyridin-7-yl]-1-piperidyl]-[4-(trifluoromethoxy)phenyl]methanone; LC/MS (m/z, M+H): 514

Example 95: 1-[4-[7-[1-[4-(trifluoromethoxy)benzoyl]-4-piperidyl]-3H-imidazo[4,5-b]pyridin-2-yl]-1-piperidyl]ethanone; LC/MS (m/z, M+H): 516

### Examples 96 and 97: [2-amino-4-(trifluoromethoxy)phenyl]-[4-[2-[morpholin-2-yl]-3H-imidazo[4,5-b]pyridin-7-yl]-1-piperidyl]methanone, Isomers 1 and 2

Examples 96 and 97 were obtained by chiral separation of the compound of Example 29 using Method CS5.

Example 97 (Isomer 2): LC/MS (m/z, M+H): 491; ¹H NMR (400 MHz, DMSO-d6, 120° C.) δ ppm 1.81-2.00 (m, 4H), 2.75-2.86 (2H hidden), 2.97-3.20 (m, 6H), 3.41-3.52 (m, 1H), 3.66 (ddd, J=11.1, 9.2, 4.0 Hz, 1H), 3.88 (dt, J=11.1, 2.9, 2.9 Hz, 1H), 4.16 (br d, J=13.4 Hz, 2H), 4.68 (dd, J=9.3, 2.9 Hz, 1H), 5.26 (br s, 2H), 6.45-6.50 (m, 1H), 6.66-6.70 (m, 1H), 7.02 (d, J=5.0 Hz, 1H), 7.14 (d, J=8.4 Hz, 1H), 8.19 (d, J=5.0 Hz, 1H)

### Example 98: [2-amino-4-(pentafluoro-λ⁶-sulfanyl)phenyl]-[4-(2-tetrahydropyran-4-yl-3H-imidazo[4,5-b]pyridin-7-yl)-1-piperidyl]methanone

Example 98 was prepared following a procedure similar to that of Example 131 using 2-amino-4-(pentafluoro-λ⁶-sulfanyl)benzoic acid;hydrochloride. LC/MS (m/z, M+H): 532; ¹H NMR (400 MHz, DMSO-d6, 100° C.) δ ppm 1.80-2.02 (m, 8H) 3.04-3.21 (m, 3H) 3.33-3.55 (m, 3H) 3.95 (dt, J=11.4, 3.5 Hz, 2H) 4.05-4.22 (m, 2H) 5.45 (br s, 2H) 6.97-7.04 (m, 2H) 7.23 (d, J=8.5 Hz, 1H) 7.27 (d, J=2.3 Hz, 1H) 8.15 (d, J=5.0 Hz, 1H) 12.13-12.47 (m, 1H)

### Example 99: (cis)-[4-(2-tetrahydropyran-4-yl-3H-imidazo[4,5-b]pyridin-7-yl)-1-piperidyl]-[4-(trifluoromethoxy)cyclohexyl]methanone

Example 99 was prepared following a procedure similar to that of step 5 of Examples 1 and 2. The cis isomer was crystallised from MeCN after purification on silica gel. LC/MS (m/z, M+H): 481

### Examples 100 and 101

Examples 100 and 101 were prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2.

Example 100: 1-[4-[7-[1-[2-amino-4-(trifluoromethoxy)benzoyl]-4-piperidyl]-3H-imidazo[4,5-b]pyridin-2-yl]-1-piperidyl]ethanone; LC/MS (m/z, M+H): 531

Example 101: [2-amino-4-(trifluoromethoxy)phenyl]-[4-[2-(1-cyclopropyl-4-piperidyl)-3H-imidazo[4,5-b]pyridin-7-yl]-1-piperidyl]methanone; LC/MS (m/z, M+H): 529; ¹H NMR (400 MHz, DMSO-d6, 120° C.) δ ppm 0.29-0.36 (m, 2H), 0.37-0.46 (m, 2H), 1.64-1.72 (m, 1H), 1.78-2.02 (m, 8H), 2.28-2.38 (m, 2H), 2.85-2.93 (partially hidden m, 1H), 2.98-3.15 (m, 4H), 3.35-3.46 (m, 1H), 4.16 (br d, J=13.2 Hz, 2H), 5.26 (br s, 2H), 6.45-6.50 (m, 1H), 6.66-6.70 (m, 1H), 6.97 (d, J=5.0 Hz, 1H), 7.14 (d, J=8.3 Hz, 1H), 8.12 (d, J=5.0 Hz, 1H), 11.49-12.40 (m, 1H)

### Example 102: [4-(2-cyclohexyl-3H-imidazo[4,5-b]pyridin-7-yl)-1-piperidyl]-[4-(trifluoromethoxy)phenyl]methanone

Example 102 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 473

### Example 103: (rac)-[2-amino-4-(trifluoromethoxy)phenyl]-[4-[2-(1,4-dioxan-2-yl)-3H-imidazo[4,5-b]pyridin-7-yl]-1-piperidyl]methanone

Example 103 was prepared following a procedure similar to that of step 1 of Example 15 using tetrahydro-2H-pyran-4-carboxylic acid and tert-butyl 4-(2,3-diamino-4-pyridyl)piperidine-1-carboxylate, followed by a procedure similar to that of step 2 of Example 293 (without MeOH) and step 3 of Example 293 (using 2-amino-4-(trifluoromethoxy)benzoic acid).

### Examples 104-120

Examples 104-120 were prepared following a procedure similar to that of step 5 of Examples 1 and 2.

Example 104: 2-[4-(2-tetrahydropyran-4-yl-3H-imidazo[4,5-b]pyridin-7-yl)piperidine-1-carbonyl]benzothiophene-5-carbonitrile; LC/MS (m/z, M+H): 472.2

Example 105: (5-amino-1-phenyl-pyrazol-4-yl)-[4-(2-tetrahydropyran-4-yl-3H-imidazo[4,5-b]pyridin-7-yl)-1-piperidyl]methanone; LC/MS (m/z, M+H): 472.3

Example 106: 2-[2-oxo-2-[4-(2-tetrahydropyran-4-yl-3H-imidazo[4,5-b]pyridin-7-yl)-1-piperidyl]ethyl]-4H-1,4-benzoxazin-3-one; LC/MS (m/z, M+H): 476.3

Example 107: 1-(3,4-difluorophenyl)-4-[4-(2-tetrahydropyran-4-yl-3H-imidazo[4,5-b]pyridin-7-yl)piperidine-1-carbonyl]pyrrolidin-2-one; LC/MS (m/z, M+H): 510.3

Example 108: [4-(3-chloro-4-fluoro-phenyl)phenyl]-[4-(2-tetrahydropyran-4-yl-3H-imidazo[4,5-b]pyridin-7-yl)-1-piperidyl]methanone; LC/MS (m/z, M+H): 519.3

Example 109: (7-amino-2-methyl-pyrazolo[1,5-a]pyrimidin-6-yl)-[4-(2-tetrahydropyran-4-yl-3H-imidazo[4,5-b]pyridin-7-yl)-1-piperidyl]methanone; LC/MS (m/z, M+H): 461.3

Example 110: 1-[(2-chlorophenyl)methyl]-4-[4-(2-tetrahydropyran-4-yl-3H-imidazo[4,5-b]pyridin-7-yl)piperidine-1-carbonyl]pyrrolidin-2-one; LC/MS (m/z, M+H): 522.3

Example 111: 1-(4-fluorophenyl)-4-[4-(2-tetrahydropyran-4-yl-3H-imidazo[4,5-b]pyridin-7-yl)piperidine-1-carbonyl]pyrrolidin-2-one; LC/MS (m/z, M+H): 492.3

Example 112: [1-[(2-chlorophenyl)methyl]pyrazol-4-yl]-[4-(2-tetrahydropyran-4-yl-3H-imidazo[4,5-b]pyridin-7-yl)-1-piperidyl]methanone; LC/MS (m/z, M+H): 505.3

Example 113: [4-(2-aminophenyl)phenyl]-[4-(2-tetrahydropyran-4-yl-3H-imidazo[4,5-b]pyridin-7-yl)-1-piperidyl]methanone; LC/MS (m/z, M+H): 482.3

Example 114: (3,6-dichloroimidazo[1,2-a]pyridin-2-yl)-[4-(2-tetrahydropyran-4-yl-3H-imidazo[4,5-b]pyridin-7-yl)-1-piperidyl]methanone; LC/MS (m/z, M+H): 499.2

Example 115: (4-amino-1-ethyl-pyrazol-3-yl)-[4-(2-tetrahydropyran-4-yl-3H-imidazo[4,5-b]pyridin-7-yl)-1-piperidyl]methanone; LC/MS (m/z, M+H): 424.3

Example 116: (3-aminoquinoxalin-2-yl)-[4-(2-tetrahydropyran-4-yl-3H-imidazo[4,5-b]pyridin-7-yl)-1-piperidyl]methanone; LC/MS (m/z, M+H): 458.3

Example 117: 2-[4-(2-tetrahydropyran-4-yl-3H-imidazo[4,5-b]pyridin-7-yl)piperidine-1-carbonyl]spiro[cyclopropane-1,3′-indoline]-2′-one; LC/MS (m/z, M+H): 472.3

Example 118: [1-(2-chlorophenyl)pyrrolidin-3-yl]-[4-(2-tetrahydropyran-4-yl-3H-imidazo[4,5-b]pyridin-7-yl)-1-piperidyl]methanone; LC/MS (m/z, M+H): 492.3

Example 119: (2-amino-4,5-dichloro-phenyl)-[4-(2-tetrahydropyran-4-yl-3H-imidazo[4,5-b]pyridin-7-yl)-1-piperidyl]methanone LC/MS (m/z, M+H): 474.2

### Example 120: [1-(4-fluorophenyl)imidazol-4-yl]-[4-(2-tetrahydropyran-4-yl-3H-imidazo[4,5-b]pyridin-7-yl)-1-piperidyl]methanone; LC/MS (m/z, M+H): 475.3 Examples 121 and 122 [2-Amino-4-(trifluoromethoxy)phenyl]-[4-[2-(1-methoxy-1-methyl-ethyl)-3H-imidazo[4,5-b]pyridin-7-yl]-1-piperidyl]methanone and [2-Amino-4-(trifluoromethoxy)phenyl]-[4-[2-(1-hydroxy-1-methyl-ethyl)-3H-imidazo[4,5-b]pyridin-7-yl]-1-piperidyl]methanone

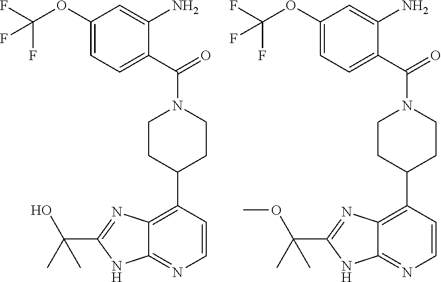

### Step 1: 4-(4-Piperidyl)pyridine-2,3-diamine;hydrochloride

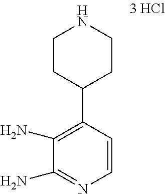 [SMILES: Nc1nccc(C2CCNCC2)c1N]

To a solution of tert-butyl 4-(2,3-diamino-4-pyridyl)piperidine-1-carboxylate (4 g, 13.7 mmol) in MeOH (150 mL) was added at 5° C. a 4 M HCl solution in dioxane (28 mL, 112 mmol). The reaction mixture was then warmed up to room temperature and stirred for 18 hours, then concentrated in vacuo. The resulting residue was then triturated with 50 mL of MeCN and 6 mL of i-PrOH. The resulting solid was then filtered off to give 4.126 g (100% yield) of 4-(4-piperidyl)pyridine-2,3-diamine;hydrochloride as a red solid.

### Step 2: [1-[7-[1-[2-(tert-Butoxycarbonylamino)-4-(trifluoromethoxy)benzoyl]-4-piperidyl]-3H-imidazo[4,5-b]pyridin-2-yl]-1-methyl-ethyl]acetate

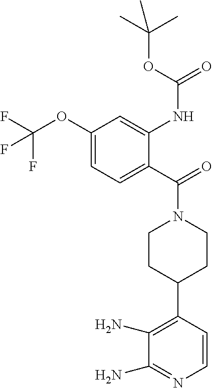 [SMILES: CC(C)(C)OC(=O)Nc1cc(OC(F)(F)F)ccc1C(=O)N1CCC(c2ccnc(N)c2N)CC1]

Step 2 was performed following the protocol described for Example 131 using 2-(tert-butoxycarbonylamino)-4-(trifluoromethoxy)benzoic acid to give 3.73 g (61% yield) of [1-[7-[1-[2-(tert-butoxycarbonylamino)-4-(trifluoromethoxy)benzoyl]-4-piperidyl]-3H-imidazo[4,5-b]pyridin-2-yl]-1-methyl-ethyl]acetate as a brown solid.

### Step 3: [1-[7-[1-[2-(tert-Butoxycarbonylamino)-4-(trifluoromethoxy)benzoyl]-4-piperidyl]-3H-imidazo[4,5-b]pyridin-2-yl]-1-methyl-ethyl]acetate

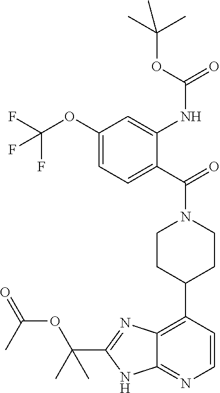 [SMILES: CC(=O)OC(C)(C)c1nc2c(C3CCN(C(=O)c4ccc(OC(F)(F)F)cc4NC(=O)OC(C)(C)C)CC3)ccnc2[nH]1]

Step 3 was performed following the protocol described in step 1 of Example 15 to give 89 mg (48% yield) of [1-[7-[1-[2-(tert-butoxycarbonylamino)-4-(trifluoromethoxy)benzoyl]-4-piperidyl]-3H-imidazo[4,5-b]pyridin-2-yl]-1-methyl-ethyl]acetate as a yellow solid.

### Step 4: [1-[7-[1-[2-Amino-4-(trifluoromethoxy)benzoyl]-4-piperidyl]-3H-imidazo[4,5-b]pyridin-2-yl]-1-methyl-ethyl]acetate

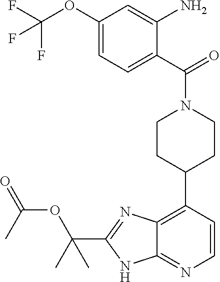 [SMILES: CC(=O)OC(C)(C)c1nc2c(C3CCN(C(=O)c4ccc(OC(F)(F)F)cc4N)CC3)ccnc2[nH]1]

To a solution of [1-[7-[1-[2-(tert-butoxycarbonylamino)-4-(trifluoromethoxy)benzoyl]-4-piperidyl]-3H-imidazo[4,5-b]pyridin-2-yl]-1-methyl-ethyl]acetate (85 mg, 0.14 mmol) in DCM (3 mL) was added at 5° C., TFA (120 μL, 1.61 mmol). The reaction mixture was then stirred at room temperature for 48 hours.

Additional TFA (120 μL, 1.61 mmol) was added and the reaction mixture was stirred for 24 additional hours, then diluted with DCM (20 mL) and water (20 mL). pH was adjusted to 10 with solid Na₂CO₃. Then, the whole was filtered on a liquid-liquid hydrophobic phase separation cartridge (hydrophobic Radleys Cartridge, 70 mL) and the resulting filtrate was concentrated in vacuo to give 89 mg (85% yield) of crude [1-[7-[1-[2-amino-4-(trifluoromethoxy)benzoyl]-4-piperidyl]-3H-imidazo[4,5-b]pyridin-2-yl]-1-methyl-ethyl]acetate as an orange solid.

### Step 5: [2-Amino-4-(trifluoromethoxy)phenyl]-[4-[2-(1-hydroxy-1-methyl-ethyl)-3H-imidazo[4,5-b]pyridin-7-yl]-1-piperidyl]methanone (Example 122) and [2-Amino-4-(trifluoromethoxy)phenyl]-[4-[2-(1-methoxy-1-methyl-ethyl)-3H-imidazo[4,5-b]pyridin-7-yl]-1-piperidyl]methanone (Example 121)

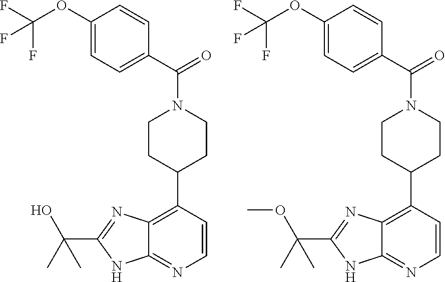

To a solution of [1-[7-[1-[2-amino-4-(trifluoromethoxy)benzoyl]-4-piperidyl]-3H-imidazo[4,5-b]pyridin-2-yl]-1-methyl-ethyl]acetate (70 mg, 0.14 mmol), in THF (1.5 mL) and MeOH (1.5 mL) was added an aqueous 1 N solution of NaOH (1 mL, 1 mmol). The resulting mixture was then stirred for 2 hours at 50° C. After 2 hours, the reaction mixture was cooled down to room temperature and concentrated in vacuo, diluted with AcOEt (20 mL) and water (20 mL), and transferred to a separating funnel. The aqueous layer was extracted with 20 mL of AcOEt and the combined organic layers were dried over sodium sulfate, filtered and concentrated to dryness. The resulting residue was purified on silica gel (SiO₂ 12 g) eluting with DCM/MeOH 95/5 to give 7 mg (11% yield) of [2-amino-4-(trifluoromethoxy)phenyl]-[4-[2-(1-hydroxy-1-methyl-ethyl)-3H-imidazo[4,5-b]pyridin-7-yl]-1-piperidyl]methanone (Example 122), and 32 mg (48% yield) of [2-amino-4-(trifluoromethoxy)phenyl]-[4-[2-(1-methoxy-1-methyl-ethyl)-3H-imidazo[4,5-b]pyridin-7-yl]-1-piperidyl]methanone (Example 121) as white solids. (hydroxy compound). (Methoxy compound).

Example 121: LC/MS (m/z, M+H, Method 1): calc. 478.5, found 478.3; ¹H NMR (400 MHz, DMSO-d6, 100° C.) mixture of rotamers 70/30—δ ppm 1.60-1.67 (m, 6H), 1.67-1.82 (m, 1H), 1.85-2.02 (m, 3H), 3.04-3.18 (m, 5H), 3.40-3.54 (m, 1H), 4.05-4.25 (m, 2H), 5.28-5.37 (m, 2H), 6.48 (br d, J=8.7 Hz, 1H), 6.68 (s, 1H), 7.00-7.07 (m, 1H), 7.10-7.24 (m, 1H), 8.17 (d, J=5.0 Hz, 0.3H), 8.26 (d, J=5.0 Hz, 0.7H), 12.09-12.24 (m, 0.3H), 12.41-12.55 (m, 0.7H)

Example 122: LC/MS (m/z, M+H, Method 1): calc. 464.4, found 464.3

### Examples 123 and 124: 5-[7-[1-[4-(trifluoromethoxy)benzoyl]-4-piperidyl]-3H-imidazo[4,5-b]pyridin-2-yl]piperidin-2-one, Isomers 1 and 2

Examples 123 and 124 were prepared following a procedure similar to that of step 1 of Example 5 using [4-(2,3-diamino-4-pyridyl)-1-piperidyl]-[4-(trifluoromethoxy)phenyl]methanone and the corresponding carboxylic acid, followed by chiral separation using Method CS7.

Example 123 (Isomer 1): LC/MS (m/z, M+H): 488

Example 124 (Isomer 2): LC/MS (m/z, M+H): 488

### Examples 125 and 126

Examples 125 and 126 were prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2.

Example 125: [2-amino-4-(trifluoromethoxy)phenyl]-[4-(2-cyclohexyl-3H-imidazo[4,5-b]pyridin-7-yl)-1-piperidyl]methanone; LC/MS (m/z, M+H): 488

Example 126: [2-amino-4-(trifluoromethoxy)phenyl]-[4-[2-(3-methoxypropyl)-3H-imidazo[4,5-b]pyridin-7-yl]-1-piperidyl]methanone; LC/MS (m/z, M+H): 478

### Example 127: [4-[2-(3-amino-1-bicyclo[1.1.1]pentanyl)-3H-imidazo[4,5-b]pyridin-7-yl]-1-piperidyl]-[4-(trifluoromethoxy)phenyl]methanone

Example 127 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 472

### Example 128: [4-[2-(3-amino-1-bicyclo[1.1.1]pentanyl)-3H-imidazo[4,5-b]pyridin-7-yl]-1-piperidyl]-[2-amino-4-(trifluoromethoxy)phenyl]methanone

Example 128 was prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2. LC/MS (m/z, M+H): 487

### Example 129: (trans)-[4-(2-tetrahydropyran-4-yl-3H-imidazo[4,5-b]pyridin-7-yl)-1-piperidyl]-[4-(trifluoromethoxy)cyclohexyl]methanone

Example 129 was prepared following a procedure similar to that of step 5 of Examples 1 and 2 using 4-(trifluoromethoxy)cyclohexanecarboxylic acid and 7-(4-piperidyl)-2-tetrahydropyran-4-yl-3H-imidazo[4,5-b]pyridine;hydrochloride (which was obtained by a procedure similar to step 2 of Example 11 using HCl instead of TFA), followed by isomeric separation by Method CS16. LC/MS (m/z, M+H): 482

### Example 130: [4-[2-[(2R)-morpholin-2-yl]-3H-imidazo[4,5-b]pyridin-7-yl]-1-piperidyl]-[4-(trifluoromethoxy)phenyl]methanone

Example 130 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 476

### Example 131 [3-fluoro-4-(trifluoromethoxy)phenyl]-[4-(2-tetrahydropyran-4-yl-3H-imidazo[4,5-b]pyridin-7-yl)-1-piperidyl]methanone

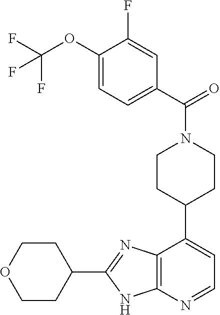 [SMILES: O=C(c1ccc(OC(F)(F)F)c(F)c1)N1CCC(c2ccnc3[nH]c(C4CCOCC4)nc23)CC1]

Under argon atmosphere, at room temperature, to a solution of 7-(4-piperidyl)-2-tetrahydropyran-4-yl-3H-imidazo[4,5-b]pyridine;hydrochloride (200 mg, 0.50 mmol) in DMF (2 mL) was added 3-fluoro-4-(trifluoromethoxy)benzoic acid (124 mg, 0.56 mmol), TBTU (195 mg, 0.61 mmol) and triethylamine 204 mg, 0.282 mL, 2.02 mmol). The resulting mixture was stirred at room temperature for 3 hours, then diluted with water, transferred to a separating funnel, and extracted twice with AcOEt. The combined organic layers were then dried over magnesium sulfate, filtered and concentrated in vacuo. The resulting residue was purified by flash chromatography (SiO₂ 12 g) eluting with DCM/MeOH 96/4 to give 106 mg (43% yield) of [3-fluoro-4-(trifluoromethoxy)phenyl]-[4-(2-tetrahydropyran-4-yl-3H-imidazo[4,5-b]pyridin-7-yl)-1-piperidyl]methanone as a pink solid. LC/MS (m/z, M+H, Method 1): calc. 493.5, found 493.3; ¹H NMR (400 MHz, DMSO-d6, 120° C.) δ ppm 1.85-2.04 (m, 8H), 3.07-3.22 (m, 3H), 3.39-3.58 (m, 3H), 3.95 (br d, J=11.6 Hz, 2H), 4.08-4.25 (m, 2H), 7.01 (d, J=5.1 Hz, 1H), 7.37 (br d, J=8.4 Hz, 1H), 7.46-7.60 (m, 2H), 8.1-8.2 (m, 1H), 11.73-12.63 (m, 1H)

### Examples 132 and 133: (cis)-[2-amino-4-(trifluoromethoxy)phenyl]-[4-[2-(3-methoxycyclobutyl)-3H-imidazo[4,5-b]pyridin-7-yl]-1-piperidyl]methanone and (trans)-[2-amino-4-(trifluoromethoxy)phenyl]-[4-[2-(3-methoxycyclobutyl)-3H-imidazo[4,5-b]pyridin-7-yl]-1-piperidyl]methanone

Examples 132 and 133 were prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2.

Example 132 (cis-isomer): LC/MS (m/z, M+H): 490

Example 133 (trans-isomer): LC/MS (m/z, M+H): 490; ¹H NMR (400 MHz, DMSO-d6, 101° C.) δ ppm 1.83-2.00 (m, 4H), 2.33-2.42 (m, 2H), 2.57-2.72 (m, 2H), 3.05-3.15 (m, 2H), 3.20 (s, 3H), 3.34-3.50 (m, 1H), 3.60-3.72 (m, 1H), 4.11-4.24 (m, 3H), 5.33 (s, 2H), 6.48 (br d, J=8.4 Hz, 1H), 6.68 (br s, 1H), 6.99 (d, J=4.9 Hz, 1H), 7.15 (d, J=8.4 Hz, 1H), 8.14 (br d, J=4.9 Hz, 1H), 11.93-12.65 (m, 1H)

### Examples 134-139

Examples 134-139 were prepared following a procedure similar to that of step 2 of Example 6.

Example 134: [4-[2-[(4-methylpiperazin-1-yl)methyl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 503.2

Example 135: [4-[2-[2-(5-chloro-2-hydroxy-phenyl)thiazol-4-yl]-3H-imidazo[4,5-b]pyridin-7-yl]-1-piperidyl]-[4-(trifluoromethoxy)phenyl]methanone; LC/MS (m/z, M+H): 600.1

Example 136: [4-[2-(2-fluoro-5-hydroxy-phenyl)-3H-imidazo[4,5-b]pyridin-7-yl]-1-piperidyl]-[4-(trifluoromethoxy)phenyl]methanone; LC/MS (m/z, M+H): 501.2

Example 137: N-[3-[7-[1-[4-(trifluoromethoxy)benzoyl]-4-piperidyl]-3H-imidazo[4,5-b]pyridin-2-yl]phenyl]tetrahydrofuran-2-carboxamide; LC/MS (m/z, M+H): 580.3

Example 138: [4-[2-[5-(hydroxymethyl)isoxazol-3-yl]-3H-imidazo[4,5-b]pyridin-7-yl]-1-piperidyl]-[4-(trifluoromethoxy)phenyl]methanone; LC/MS (m/z, M+H): 488.2

Example 139: 1-ethyl-3-hydroxy-6-[7-[1-[4-(trifluoromethoxy)benzoyl]-4-piperidyl]-3H-imidazo[4,5-b]pyridin-2-yl]quinoxalin-2-one; LC/MS (m/z, M+H): 579.2

### Example 140: (rac)-[4-[2-(3-piperidyl)-3H-imidazo[4,5-b]pyridin-7-yl]-1-piperidyl]-[4-(trifluoromethoxy)phenyl]methanone

Example 140 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 474

### Example 141: (rac)-[3-(2-tetrahydropyran-4-yl-3H-imidazo[4,5-b]pyridin-7-yl)pyrrolidin-1-yl]-[4-(trifluoromethoxy)phenyl]methanone

Example 141 was prepared following a procedure similar to that of Examples 332 and 333 using tert-butyl 3-(4,4,5,5-tetramethyl-1,3,2-dioxaborolan-2-yl)-2,5-dihydro-1H-pyrrole-1-carboxylate in step 1, and using 4-(trifluoromethoxy)benzoic acid in step 4.

### Example 142: (rac)-[2-amino-4-(trifluoromethoxy)phenyl]-[3-(2-tetrahydropyran-4-yl-3H-imidazo[4,5-b]pyridin-7-yl)pyrrolidin-1-yl]methanone

Example 142 was prepared following a procedure similar to that of Examples 332 and 333 using tert-butyl 3-(4,4,5,5-tetramethyl-1,3,2-dioxaborolan-2-yl)-2,5-dihydro-1H-pyrrole-1-carboxylate in step 1, and using 2-amino-4-(trifluoromethoxy)benzoic acid in step 4.

### Example 143: (4-bromophenyl)-[4-(2-tetrahydropyran-4-yl-3H-imidazo[4,5-b]pyridin-7-yl)-1-piperidyl]methanone

Example 143 was prepared following a procedure similar to that of Example 131 using 4-bromobenzoic acid. LC/MS (m/z, M+H): 469.2

### Examples 144 and 145: [2-amino-4-(trifluoromethoxy)phenyl]-[4-[2-[1,4-dioxan-2-yl]-3H-imidazo[4,5-b]pyridin-7-yl]-1-piperidyl]methanone, Isomers 1 and 2

Examples 144 and 145 were obtained by chiral separation of the compound of Example 103 using Method CS13.

Example 144 (Isomer 1): LC/MS (m/z, M+H): 492

### Example 146: 4-[7-[1-[2-amino-4-(trifluoromethoxy)benzoyl]-4-piperidyl]-3H-imidazo[4,5-b]pyridin-2-yl]cyclohexanone

Example 146 was prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2. LC/MS (m/z, M+H): 502

### Example 147: [4-(2-tetrahydropyran-4-yl-3H-imidazo[4,5-b]pyridin-7-yl)-1-piperidyl]-(4-trimethylsilylphenyl)methanone

Example 147 was prepared by a procedure similar to that of Example 131 using 4-trimethylsilylbenzoic acid and 7-(4-piperidyl)-2-tetrahydropyran-4-yl-3H-imidazo[4,5-b]pyridine;hydrochloride (prepared by a procedure similar to that of step 2 of Example 11 using HCl instead of TFA).

### Examples 148 and 149: (cis)-[4-[2-(3-hydroxycyclobutyl)-3H-imidazo[4,5-b]pyridin-7-yl]-1-piperidyl]-[4-(trifluoromethoxy)phenyl]methanone and (trans)-[4-[2-(3-hydroxycyclobutyl)-3H-imidazo[4,5-b]pyridin-7-yl]-1-piperidyl]-[4-(trifluoromethoxy)phenyl]methanone

Examples 148 and 149 were prepared by a procedure similar to that of Example 170 using Intermediate I and [4-(2,3-diamino-4-pyridyl)-1-piperidyl]-[4-(trifluoromethoxy)phenyl]methanone (prepared according to step 1 of Example 6) in step 1.

Example 148 (cis-isomer): LC/MS (m/z, M+H): 461

Example 149 (trans-isomer): LC/MS (m/z, M+H): 461

### Example 150: [2-amino-4-(trifluoromethoxy)phenyl]-[4-[2-(3-methoxy-1-bicyclo[1.1.1]pentanyl)-3H-imidazo[4,5-b]pyridin-7-yl]-1-piperidyl]methanone

Example 150 was prepared following a procedure similar to that of step 1 of Example 15 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2. LC/MS (m/z, M+H): 502; ¹H NMR (400 MHz, DMSO-d6, 120° C.) δ ppm 1.83-1.98 (m, 4H), 2.30 (s, 6H), 3.05-3.15 (m, 2H), 3.29 (s, 3H), 3.38-3.49 (m, 1H), 4.16 (br d, J=13.3 Hz, 2H), 5.26 (br s, 2H), 6.44-6.51 (m, 1H), 6.66-6.72 (m, 1H), 7.00 (d, J=5.0 Hz, 1H), 7.14 (d, J=8.4 Hz, 1H), 8.15 (br d, J=5 Hz, 1H), 12.09-12.70 (m, 1H)

### Examples 151 and 152: 5-[7-[1-[2-amino-4-(trifluoromethoxy)benzoyl]-4-piperidyl]-3H-imidazo[4,5-b]pyridin-2-yl]piperidin-2-one, Isomers 1 and 2

Examples 151 and 152 were prepared following a procedure similar to that of step 1 of Example 5 using [2-amino-4-(trifluoromethoxy)phenyl]-[4-(2,3-diamino-4-pyridyl)-1-piperidyl]methanone and the corresponding carboxylic acid, followed by chiral separation using Method CS4.

Example 151 (Isomer 1): LC/MS (m/z, M+H): 503; ¹H NMR (400 MHz, DMSO-d6, 120° C.) δ ppm 1.87-1.99 (m, 4H), 2.11-2.37 (m, 4H), 3.04-3.16 (m, 2H), 3.29-3.44 (m, 2H), 3.58 (dd, J=7.5, 2.3 Hz, 2H), 4.11 (br d, J=13.6 Hz, 2H), 5.26 (br s, 2H), 6.48 (br d, J=8.3 Hz, 1H), 6.64-6.77 (m, 1H), 7.00 (d, J=5.0 Hz, 1H), 7.02-7.07 (m, 1H), 7.14 (d, J=8.3 Hz, 1H), 8.15 (d, J=5.0 Hz, 1H)

Example 152 (Isomer 2): LC/MS (m/z, M+H): 503

### Examples 153 and 154: (trans)-[4-(trifluoromethoxy)phenyl]-[4-[2-(4-methoxycyclohexyl)-3H-imidazo[4,5-b]pyridin-7-yl]-1-piperidyl]methanone;hydrochloride and (cis)-[4-(trifluoromethoxy)phenyl]-[4-[2-(4-methoxycyclohexyl)-3H-imidazo[4,5-b]pyridin-7-yl]-1-piperidyl]methanone;hydrochloride

Examples 153 and 154 were prepared following a procedure similar to that of step 1 of Example 15 using [4-(2,3-diamino-4-pyridyl)-1-piperidyl]-[4-(trifluoromethoxy)phenyl]methanone and the corresponding carboxylic acid, followed by isomeric separation using Method CS8.

Example 154 (cis-isomer): LC/MS (m/z, M+H): 503

### Examples 155 and 156: (trans)-[2-amino-4-(trifluoromethoxy)phenyl]-[4-[2-(4-methoxycyclohexyl)-3H-imidazo[4,5-b]pyridin-7-yl]-1-piperidyl]methanone;hydrochloride and (cis)-[2-amino-4-(trifluoromethoxy)phenyl]-[4-[2-(4-methoxycyclohexyl)-3H-imidazo[4,5-b]pyridin-7-yl]-1-piperidyl]methanone;hydrochloride

Examples 155 and 156 were prepared following a procedure similar to that of step 1 of Example 15 using tert-butyl N-[2-[4-(2,3-diamino-4-pyridyl)piperidine-1-carbonyl]-5-(trifluoromethoxy)phenyl]carbamate and the corresponding carboxylic acid, followed by isomeric separation using Method CS8.

Example 156: LC/MS (m/z, M+H): 518; ¹H NMR (400 MHz, DMSO-d6, 120° C.) δ ppm 1.29-1.38 (m, 2H), 1.65-1.77 (m, 2H), 1.85-1.99 (m, 4H), 2.07-2.15 (m, 4H), 2.84-2.89 (m, 1H), 3.05-3.15 (m, 2H), 3.18-3.25 (m, 1H), 3.28 (s, 3H), 3.35-3.45 (m, 1H), 4.16 (br d, J=13.1 Hz, 2H), 5.26 (br s, 2H), 6.47 (br d, J=8.3 Hz, 1H), 6.64-6.71 (m, 1H), 6.97 (d, J=5.0 Hz, 1H), 7.14 (d, J=8.3 Hz, 1H), 8.12 (br d, J=5.0 Hz, 1H), 11.73-12.53 (m, 1H)

### Examples 157-160

Examples 157-160 were prepared following a procedure similar to that of step 5 of Examples 1 and 2.

Example 157: (2,2-difluoro-1,3-benzodioxol-5-yl)-[4-(2-tetrahydropyran-4-yl-3H-imidazo[4,5-b]pyridin-7-yl)-1-piperidyl]methanone; LC/MS (m/z, M+H): 471

Example 158: (4-bromo-1H-indol-7-yl)-[4-(2-tetrahydropyran-4-yl-3H-imidazo[4,5-b]pyridin-7-yl)-1-piperidyl]methanone; LC/MS (m/z, M+H):

Example 159: [4-(2-tetrahydropyran-4-yl-3H-imidazo[4,5-b]pyridin-7-yl)-1-piperidyl]-[2-(trifluoromethyl)-1H-indol-6-yl]methanone; LC/MS (m/z, M+H): 498

Example 160: [4-(cyclopropoxy)phenyl]-[4-(2-tetrahydropyran-4-yl-3H-imidazo[4,5-b]pyridin-7-yl)-1-piperidyl]methanone; LC/MS (m/z, M+H): 447

### Examples 161 and 162: [2-amino-4-(trifluoromethoxy)phenyl]-[4-[2-[5,5-difluoro-3-piperidyl]-3H-imidazo[4,5-b]pyridin-7-yl]-1-piperidyl]methanone, Isomers 1 and 2

Examples 161 and 162 were obtained by chiral separation of the compound of Example 36 using Method CS4.

Example 161 (Isomer 1): LC/MS (m/z, M+H): 525

### Examples 163 and 164: [2-amino-4-(trifluoromethoxy)phenyl]-[3-(2-tetrahydropyran-4-yl-3H-imidazo[4,5-b]pyridin-7-yl)pyrrolidin-1-yl]methanone, Isomers 1 and 2

Examples 163 and 164 were obtained by chiral separation of the compound of Example 142 using Method CS1 (column (5 μm, 30×250 mm), eluting with (n-Heptane 60% EtOH 40%)+0.1% TEA (flow rate 40 mL/min)).

### Example 165: 2-tetrahydropyran-4-yl-7-[1-[4-(trifluoromethoxy)phenyl]sulfonyl-4-piperidyl]-3H-imidazo[4,5-b]pyridine

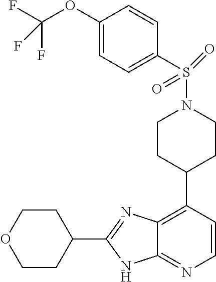 [SMILES: O=S(=O)(c1ccc(OC(F)(F)F)cc1)N1CCC(c2ccnc3[nH]c(C4CCOCC4)nc23)CC1]

Under argon atmosphere, to a solution of 7-(4-piperidyl)-2-tetrahydropyran-4-yl-3H-imidazo[4,5-b]pyridine (obtained from basic work up of the intermediate described in step 2 of Example 11, 50 mg, 0.17 mmol) in THF (1 mL) was added N,N-diisopropylethylamine (113 mg, 0.87 mmol) followed by 4-(trifluoromethoxy)benzenesulfonyl chloride (33 mg, 0.18 mmol). The whole mixture was stirred at room temperature for 6 hours. After 6 hours, the mixture was diluted with AcOEt and water, transferred to a separating funnel, and extracted twice with AcOEt. The combined organic layers were then washed with an aqueous saturated solution of NaHCO₃, dried over magnesium sulfate, filtered and concentrated in vacuo. The resulting residue was purified on silica gel (SiO₂ 20 g) eluting with DCM/(DCM/MeOH 90/10) 70/30, to give 51 mg (57% yield) of 2-tetrahydropyran-4-yl-7-[1-[4-(trifluoromethoxy)phenyl]sulfonyl-4-piperidyl]-3H-imidazo[4,5-b]pyridine as a white powder. LC/MS (m/z, M+H, Method 1): calc. 511.2, found 511.3.

### Examples 166 and 167: [2-amino-4-(trifluoromethoxy)phenyl]-[4-[2-[oxepan-4-yl]-3H-imidazo[4,5-b]pyridin-7-yl]-1-piperidyl]methanone, Isomers 1 and 2

Examples 166 and 167 were obtained by chiral separation of the compound of Example 44 using Method CS6.

Example 166 (Isomer 1): LC/MS (m/z, M+H): 504

Example 167 (Isomer 2): LC/MS (m/z, M+H): 504

### Example 168: [4-[2-[rac-(2R,3S)-3-aminotetrahydrofuran-2-yl]-3H-imidazo[4,5-b]pyridin-7-yl]-1-piperidyl]-[4-(trifluoromethoxy)phenyl]methanone;hydrochloride

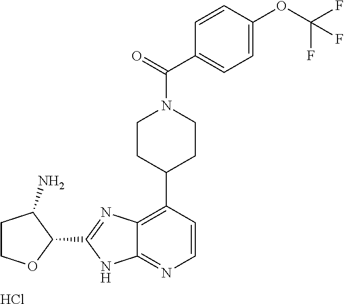 [SMILES: N[C@H]1CCO[C@H]1c1nc2c(C3CCN(C(=O)c4ccc(OC(F)(F)F)cc4)CC3)ccnc2[nH]1 ; Cl]

In a microwave vial, to a solution of (rac)-(2S,3R)-3-(tert-butoxycarbonylamino)tetrahydrofuran-2-carboxylic acid (122 mg, 0.52 mmol) in MeCN (20 mL, was added CDI (94 mg, 0.58 mmol), and the vial was sealed and submitted to microwave at 120° C. for 15 minutes. Then, [4-(2,3-diamino-4-pyridyl)-1-piperidyl]-[4-(trifluoromethoxy)phenyl]methanone (described in step 1 of Example 6; 200 mg, 0.52 mmol), the tube was sealed and the reaction mixture was submitted to microwave for 45 minutes at 130° C. Then, the reaction mixture was transferred to a 50 mL flask and 10 mL of acetic acid was added. The whole was refluxed for one hour. 5 mL of a 2 N HCl solution in Et₂O was added and the mixture was stirred for 12 hours at room temperature, then concentrated in vacuo. The resulting residue was purified on silica gel (SiO₂ 20 g), eluting with DCM/(MeOH/NH₄OH 90/10) 90/10 to 50/50 to give a residue which was taken with a solution of HCl 2 N in Et₂O, filtered to give 156 mg (54% yield) of [4-[2-[rac-(2R,3S)-3-aminotetrahydrofuran-2-yl]-3H-imidazo[4,5-b]pyridin-7-yl]-1-piperidyl]-[4-(trifluoromethoxy)phenyl]methanone;hydrochloride as a yellow solid. LC/MS (m/z, M+H-2 HCl, Method 1): calc. 476.2, found 476.3.

### Examples 169 and 170: (cis)-[2-amino-4-(trifluoromethoxy)phenyl]-[4-[2-(3-hydroxycyclobutyl)-3H-imidazo[4,5-b]pyridin-7-yl]-1-piperidyl]methanone and (trans)-[2-amino-4-(trifluoromethoxy)phenyl]-[4-[2-(3-hydroxycyclobutyl)-3H-imidazo[4,5-b]pyridin-7-yl]-1-piperidyl]methanone

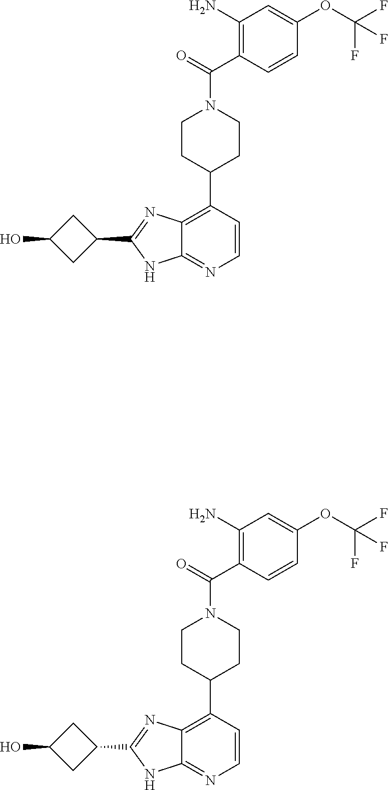 [SMILES: Nc1cc(OC(F)(F)F)ccc1C(=O)N1CCC(c2ccnc3[nH]c([C@H]4C[C@@H](O)C4)nc23)CC1 ; Nc1cc(OC(F)(F)F)ccc1C(=O)N1CCC(c2ccnc3[nH]c([C@H]4C[C@H](O)C4)nc23)CC1]

### Step 1: tert-Butyl N-[2-[4-[2-[3-[tert-butyl(dimethyl)silyl]oxycyclobutyl]-3H-imidazo[4,5-b]pyridin-7-yl]piperidine-1-carbonyl]-5-(trifluoromethoxy)phenyl]carbamate

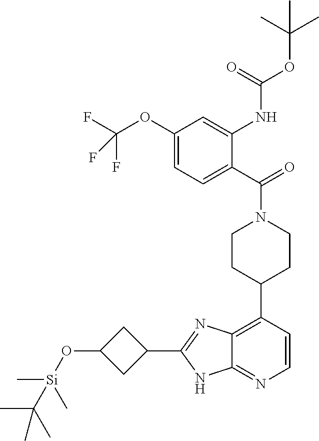 [SMILES: CC(C)(C)OC(=O)Nc1cc(OC(F)(F)F)ccc1C(=O)N1CCC(c2ccnc3[nH]c(C4CC(O[Si](C)(C)C(C)(C)C)C4)nc23)CC1]

Step 1 was performed following the protocol described in step 1 of Example 20 using intermediate I and tert-butyl N-[2-[4-(2,3-diamino-4-pyridyl)piperidine-1-carbonyl]-5-(trifluoromethoxy)phenyl]carbamate (as described in step 2 of Examples 76 and 77) to give 446 mg (70% yield) of tert-butyl N-[2-[4-[2-[3-[tert-butyl(dimethyl)silyl]oxycyclobutyl]-3H-imidazo[4,5-b]pyridin-7-yl]piperidine-1-carbonyl]-5-(trifluoromethoxy)phenyl]carbamate as white powder.

### Step 2: (trans)-tert-Butyl N-[2-[4-[2-(3-hydroxycyclobutyl)-3H-imidazo[4,5-b]pyridin-7-yl]piperidine-1-carbonyl]-5-(trifluoromethoxy)phenyl]carbamate and (cis)-tert-Butyl N-[2-[4-[2-(3-hydroxycyclobutyl)-3H-imidazo[4,5-b]pyridin-7-yl]piperidine-1-carbonyl]-5-(trifluoromethoxy)phenyl]carbamate

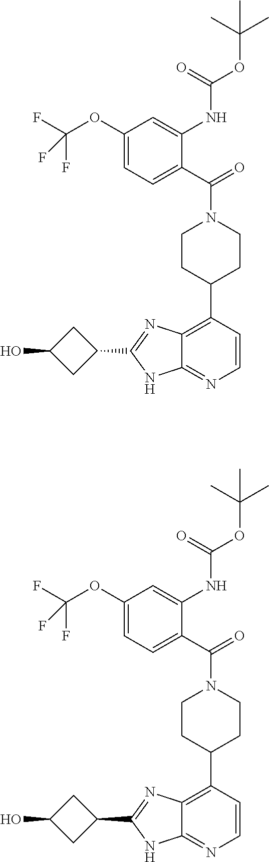 [SMILES: CC(C)(C)OC(=O)Nc1cc(OC(F)(F)F)ccc1C(=O)N1CCC(c2ccnc3[nH]c([C@H]4C[C@H](O)C4)nc23)CC1 ; CC(C)(C)OC(=O)Nc1cc(OC(F)(F)F)ccc1C(=O)N1CCC(c2ccnc3[nH]c([C@H]4C[C@@H](O)C4)nc23)CC1]

To a solution of tert-butyl N-[2-[4-[2-[3-[tert-butyl(dimethyl)silyl]oxycyclobutyl]-3H-imidazo[4,5-b]pyridin-7-yl]piperidine-1-carbonyl]-5-(trifluoromethoxy)phenyl]carbamate (446 mg, 0.65 mmol) in THF (6 mL) was added at room temperature under argon atmosphere, a 1 M solution of TBAF in THF (0.77 mL, 0.77 mmol) and the resulting reaction mixture was stirred at room temperature for 12 hours, then diluted with AcOEt, transferred to a separating funnel, and washed with brine (three times). The organic layer was then dried over sodium sulfate, filtered and concentrated. The resulting residue was purified on silica gel (SiO₂ 40 g) eluting with DCM/MeOH 92.5/7.5 to give 264 mg (71% yield) of (cis)-tert-butyl N-[2-[4-[2-[3-[tert-butyl(dimethyl)silyl]oxycyclobutyl]-3H-imidazo[4,5-b]pyridin-7-yl]piperidine-1-carbonyl]-5-(trifluoromethoxy)phenyl]carbamate and 40 mg (10% yield) of (trans)-tert-butyl N-[2-[4-[2-[3-[tert-butyl(dimethyl)silyl]oxycyclobutyl]-3H-imidazo[4,5-b]pyridin-7-yl]piperidine-1-carbonyl]-5-(trifluoromethoxy)phenyl]carbamate as white solids.

### Step 3: (trans)-[2-amino-4-(trifluoromethoxy)phenyl]-[4-[2-(3-hydroxycyclobutyl)-3H-imidazo[4,5-b]pyridin-7-yl]-1-piperidyl]methanone

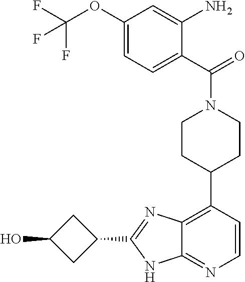 [SMILES: Nc1cc(OC(F)(F)F)ccc1C(=O)N1CCC(c2ccnc3[nH]c([C@H]4C[C@H](O)C4)nc23)CC1]

Step 3 was performed following the protocol described in step 7 of Examples 355 and 356 using (trans)-tert-butyl N-[2-[4-[2-[3-[tert-butyl(dimethyl)silyl]oxycyclobutyl]-3H-imidazo[4,5-b]pyridin-7-yl]piperidine-1-carbonyl]-5-(trifluoromethoxy)phenyl]carbamate to give 20 mg (65% yield) of (trans)-[2-amino-4-(trifluoromethoxy)phenyl]-[4-[2-(3-hydroxycyclobutyl)-3H-imidazo[4,5-b]pyridin-7-yl]-1-piperidyl]methanone as a white powder. LC/MS (m/z, M+H, Method 1): calc. 476.2, found 476.2; ¹H NMR (400 MHz, DMSO-d6, 101° C.) δ ppm 1.83-1.99 (m, 4H), 2.27-2.37 (m, 2H), 2.60-2.68 (partially hidden m, 2H), 3.05-3.15 (m, 2H), 3.35-3.46 (m, 1H), 3.63 (spt, J=4.7 Hz, 1H), 4.17 (br d, J=12.6 Hz, 2H), 4.46 (br quin, J=6.7 Hz, 1H), 4.55-6.06 (m, 3H), 6.48 (br d, J=8.3 Hz, 1H), 6.66-6.71 (m, 1H), 7.00 (d, J=4.9 Hz, 1H), 7.15 (d, J=8.3 Hz, 1H), 8.14 (d, J=4.9 Hz, 1H), 11.08-13.16 (m, 1H)

Example 169 (cis-isomer) was prepared according to step 3 above using (cis)-tert-butyl N-[2-[4-[2-(3-hydroxycyclobutyl)-3H-imidazo[4,5-b]pyridin-7-yl]piperidine-1-carbonyl]-5-(trifluoromethoxy)phenyl]carbamate (obtained in step 2). LC/MS (m/z, M+H): 476

Examples 171 and 172: [3-(2-tetrahydropyran-4-yl-3H-imidazo[4,5-b]pyridin-7-yl)pyrrolidin-1-yl]-[4-(trifluoromethoxy)phenyl]methanone, Isomers 1 and 2

Examples 171 and 172 were obtained by chiral separation of the compound of Example 141 using Method CS3 (Flow rate 40 mL/min).

### Example 173: [2-amino-4-(trifluoromethoxy)phenyl]-[4-(6-fluoro-2-tetrahydropyran-4-yl-3H-imidazo[4,5-b]pyridin-7-yl)-1-piperidyl]methanone

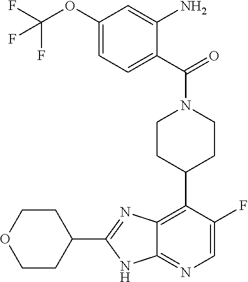 [SMILES: Nc1cc(OC(F)(F)F)ccc1C(=O)N1CCC(c2c(F)cnc3[nH]c(C4CCOCC4)nc23)CC1]

### Step 1: 6-Fluoro-2-tetrahydropyran-4-yl-3H-imidazo[4,5-b]pyridine

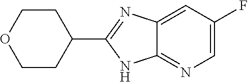 [SMILES: Fc1cnc2[nH]c(C3CCOCC3)nc2c1]

In a 10-20 mL microwave vial, were placed tetrahydropyran-4-carbaldehyde (920 mg, 8.06 mmol), dimethylformamide (10 mL), 5-fluoropyridine-2,3-diamine (1 g, 7.86 mmol) and sodium metabisulfite (1.95 g, 10.30 mmol). The vial was sealed and submitted to microwave at 125° C. for 3.25 hours. Then the reaction mixture was cooled down to room temperature, and concentrated to dryness. The resulting residue was purified by flash chromatography on silica gel (SiO₂ 40 g) eluting with DCM/MeOH 100/0 to 95/5 to give 1.10 g (63% yield) of 6-fluoro-2-tetrahydropyran-4-yl-3H-imidazo[4,5-b]pyridine as an orange solid. LC/MS (m/z, M+H): 222

### Step 2: 2-[(6-Fluoro-2-tetrahydropyran-4-yl-imidazo[4,5-b]pyridin-3-yl)methoxy]ethyl-trimethyl-silane

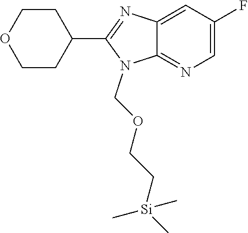 [SMILES: C[Si](C)(C)CCOCn1c(C2CCOCC2)nc2cc(F)cnc21]

Under argon atmosphere, a solution of 6-fluoro-2-tetrahydropyran-4-yl-3H-imidazo[4,5-b]pyridine (970 mg, 4.38 mmol) in dimethylformamide (15 mL) was cooled down to 5° C., and then sodium hydride (60% in oil, 250 mg, 6.25 mmol) was added portionwise. The mixture was stirred at 5° C. for 30 minutes before 2-(trimethylsilyl)ethoxymethyl chloride (1 mL, 5.70 mmol) was added dropwise. The resulting reaction mixture was stirred at 5° C. for 1.25 hours and then hydrolyzed with an aqueous saturated solution of NH₄Cl. The reaction mixture was diluted with water and was extracted twice with EtOAc. The combined organic layers were dried over magnesium sulfate, filtered and concentrated to dryness. The resulting residue was purified by flash chromatography on silica gel (SiO₂ 40 g) eluting with cyclohexane/EtOAc 80/20 to give 813 mg (52% yield) of 2-[(6-fluoro-2-tetrahydropyran-4-yl-imidazo[4,5-b]pyridin-3-yl)methoxy]ethyl-trimethyl-silane as an orange oil. LC/MS (m/z, M+H): 352

### Step 3: tert-Butyl 4-[6-fluoro-2-tetrahydropyran-4-yl-3-(2-trimethylsilylethoxymethyl)imidazo[4,5-b]pyridin-7-yl]piperidine-1-carboxylate

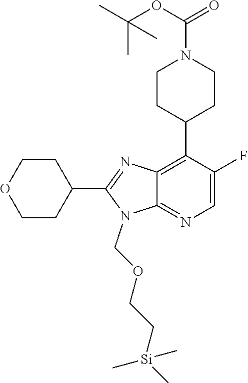 [SMILES: CC(C)(C)OC(=O)N1CCC(c2c(F)cnc3c2nc(C2CCOCC2)n3COCC[Si](C)(C)C)CC1]

In a 4 mL photochemistry vial, were placed 01-tert-butyl 04-(1,3-dioxoisoindolin-2-yl) piperidine-1,4-dicarboxylate (420 mg, 2.19 mmol), 2-[(6-fluoro-2-tetrahydropyran-4-yl-imidazo[4,5-b]pyridin-3-yl)methoxy]ethyl-trimethyl-silane (180 mg, 0.51 mmol), sodium iodide (80 mg, 0.53 mmol), and triphenylphosphine (130 mg, 0.49 mmol). The vial was evacuated and filled with argon (three times) and then a solution of trifluoroacetic acid (50 μL, 0.65 mmol) in 2 mL of acetone was added. The reaction mixture was stirred under irradiation with blue LEDs (450 nm, maintained at approximately room temperature by a desk fan). After 12 hours, the reaction mixture was filtered and concentrated to dryness. The resulting residue was triturated with MeOH, filtered and the filtrate was concentrated to dryness. The resulting residue was purified by HPLC with the following conditions: (Column, C18 SunFire (Waters)—5 μm—30×100 mm; mobile phase, water (+0.1% HCO₂H) and MeCN (+0.1% HCO₂H); Detector diode array 200-400 nm) to give 40 mg (14% yield) of tert-butyl 4-[6-fluoro-2-tetrahydropyran-4-yl-3-(2-trimethylsilylethoxymethyl)imidazo[4,5-b]pyridin-7-yl]piperidine-1-carboxylate as a colorless wax. LC/MS (m/z, M+H): 535

### Step 4: 6-Fluoro-7-(4-piperidyl)-2-tetrahydropyran-4-yl-3H-imidazo[4,5-b]pyridine;hydrochloride

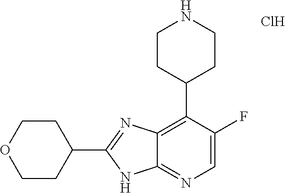 [SMILES: Fc1cnc2[nH]c(C3CCOCC3)nc2c1C1CCNCC1 ; Cl]

To a solution of tert-butyl 4-[6-fluoro-2-tetrahydropyran-4-yl-3-(2-trimethylsilylethoxymethyl)imidazo[4,5-b]pyridin-7-yl]piperidine-1-carboxylate (58 mg, 0.11 mmol) in 2 mL of MeOH was added a solution of HCl (4N in dioxane, 1 mL, 4 mmol). The resulting solution was stirred overnight and then concentrated to dryness. The residue (46 mg) was 6-fluoro-7-(4-piperidyl)-2-tetrahydropyran-4-yl-3H-imidazo[4,5-b]pyridine;hydrochloride which was used without further purification. LC/MS (m/z, M+H): 305

### Step 5: [2-Amino-4-(trifluoromethoxy)phenyl]-[4-(6-fluoro-2-tetrahydropyran-4-yl-3H-imidazo[4,5-b]pyridin-7-yl)-1-piperidyl]methanone

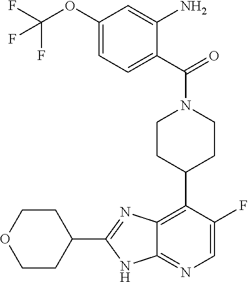 [SMILES: Nc1cc(OC(F)(F)F)ccc1C(=O)N1CCC(c2c(F)cnc3[nH]c(C4CCOCC4)nc23)CC1]

To a solution of 6-fluoro-7-(4-piperidyl)-2-tetrahydropyran-4-yl-3H-imidazo[4,5-b]pyridine;hydrochloride (46 mg, 0.11 mmol) and 2-amino-4-(trifluoromethoxy)benzoic acid (36 mg, 0.16 mmol) in DMF (3 mL) were added triethylamine (90 μL, 0.65 mmol) and TBTU (55 mg, 0.17 mmol). The resulting mixture was stirred at room temperature for 1 hour, then diluted with water, transferred to a separating funnel, and extracted twice with EtOAc. The combined organic layers were then dried over magnesium sulfate, filtered and concentrated in vacuo. The resulting residue was purified by flash chromatography (SiO₂ 12 g) eluting with DCM/MeOH 100/0 to 95/5 to give 37 mg (66% yield) of [2-amino-4-(trifluoromethoxy)phenyl]-[4-(6-fluoro-2-tetrahydropyran-4-yl-3H-imidazo[4,5-b]pyridin-7-yl)-1-piperidyl]methanone as a white solid. LC/MS (m/z, M+H, Method 1): calc. 508.2, found 508.3; ¹H NMR (400 MHz, DMSO-d6, 101° C.) δ ppm 1.81 (br d, J=12.8 Hz, 2H), 1.85-2.02 (m, 4H), 2.22-2.39 (m, 2H), 3.03-3.13 (m, 2H), 3.13-3.22 (m, 1H), 3.46-3.60 (m, 3H), 3.95 (dt, J=11.5, 3.5 Hz, 2H), 4.18 (br d, J=12.3 Hz, 2H), 5.33 (s, 2H), 6.49 (br d, J=8.3 Hz, 1H), 6.69 (br s, 1H), 7.14 (d, J=8.3 Hz, 1H), 8.11 (d, J=3.3 Hz, 1H), 12.35-12.56 (m, 1H)

### Example 174: [2-Amino-4-(trifluoromethoxy)phenyl]-[4-(2-morpholino-3H-imidazo[4,5-b]pyridin-7-yl)-1-piperidyl]methanone

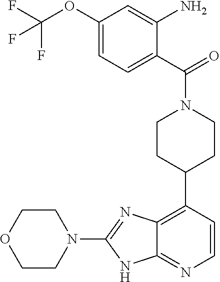 [SMILES: Nc1cc(OC(F)(F)F)ccc1C(=O)N1CCC(c2ccnc3[nH]c(N4CCOCC4)nc23)CC1]

### Step 1: tert-Butyl 4-(2-thioxo-1,3-dihydroimidazo[4,5-b]pyridin-7-yl)piperidine-1-carboxylate

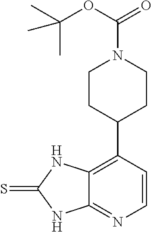 [SMILES: CC(C)(C)OC(=O)N1CCC(c2ccnc3[nH]c(=S)[nH]c23)CC1]

To a solution of tert-butyl 4-(2,3-diamino-4-pyridyl)piperidine-1-carboxylate (as described in step 2 of Examples 1 and 2; 500 mg, 1.71 mmol) in 2-methyltetrahydrofuran (10 mL) was added di(imidazol-1-yl)methanethione (305 mg, 1.71 mmol) and the resulting reaction mixture was stirred at room temperature for 12 hours. Then, additional di(imidazol-1-yl)methanethione (102 mg, 0.57 mmol) was added and the reaction mixture was stirred for two additional hours at room temperature, then diluted with AcOEt and water, and transferred to a separating funnel. The organic layer was dried over magnesium sulfate, filtered and concentrated in vacuo. The resulting residue was purified on silica gel (SiO₂ 50 g) eluting with DCM/MeOH/NH₄OH 100/0/0 to 90/10/1 to give 260 mg (45% yield) of tert-butyl 4-(2-thioxo-1,3-dihydroimidazo[4,5-b]pyridin-7-yl)piperidine-1-carboxylate as pale brown solid.

### Step 2: tert-butyl 4-(2-bromo-3H-imidazo[4,5-b]pyridin-7-yl)piperidine-1-carboxylate

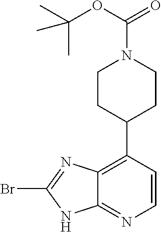 [SMILES: CC(C)(C)OC(=O)N1CCC(c2ccnc3[nH]c(Br)nc23)CC1]

To a solution of tert-butyl 4-(2-thioxo-1,3-dihydroimidazo[4,5-b]pyridin-7-yl)piperidine-1-carboxylate (270 mg, 0.81 mmol) in THF (6 mL), was added at a temperature between −10° C. and −20° C., hydrobromic acid (270 μL, 2.39 mmol) and bromine (270 μL, 5.27 mmol) and the resulting mixture was stirred between −10° C. and −20° C. for 30 minutes. The reaction mixture was diluted with AcOEt, transferred to a separating funnel, and washed with a 1 M solution of Na₂S2O₃, then with an aqueous saturated solution of NaHCO₃. The organic layer was dried over magnesium sulfate, filtered and concentrated in vacuo. The resulting residue was purified on silica gel (SiO₂ 20 g) eluting with DCM/MeOH/NH₄OH 100/0/0 to 90/10/1 to give 100 mg (32% yield) of tert-butyl 4-(2-bromo-3H-imidazo[4,5-b]pyridin-7-yl)piperidine-1-carboxylate as a yellow solid.

### Step 3: tert-butyl 4-(2-morpholino-3H-imidazo[4,5-b]pyridin-7-yl)piperidine-1-carboxylate

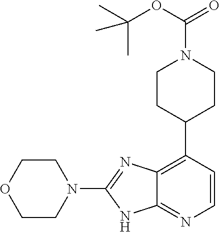 [SMILES: CC(C)(C)OC(=O)N1CCC(c2ccnc3[nH]c(N4CCOCC4)nc23)CC1]

In a microwave vial, to tert-butyl 4-(2-bromo-3H-imidazo[4,5-b]pyridin-7-yl)piperidine-1-carboxylate (30 mg, 0.079 mmol) was added morpholine (1988 mg, 22.8 mmol). The vial was sealed, submitted to microwave for 15 minutes at 170° C. Then, the reaction mixture was cooled down to room temperature, diluted with DCM and water, transferred to a separating funnel, and extracted with DCM. The combined organic layers were dried over magnesium sulfate, filtered, and concentrated in vacuo to give 30 mg (100% yield) of crude tert-butyl 4-(2-morpholino-3H-imidazo[4,5-b]pyridin-7-yl)piperidine-1-carboxylate as a yellow solid which was used in the next step without further purification.

### Step 4: 4-[7-(4-Piperidyl)-3H-imidazo[4,5-b]pyridin-2-yl]morpholine;hydrochloride

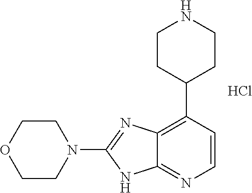 [SMILES: c1cc(C2CCNCC2)c2nc(N3CCOCC3)[nH]c2n1 ; Cl]

Step 4 was performed following the protocol described in step 3 of Example 29 to give 42 mg (100% yield) of 4-[7-(4-piperidyl)-3H-imidazo[4,5-b]pyridin-2-yl]morpholine;hydrochloride as a pale yellow solid.

### Step 5: [2-Amino-4-(trifluoromethoxy)phenyl]-[4-(2-morpholino-3H-imidazo[4,5-b]pyridin-7-yl)-1-piperidyl]methanone

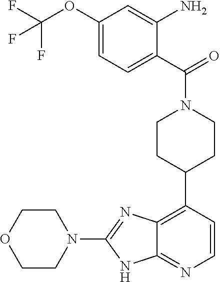 [SMILES: Nc1cc(OC(F)(F)F)ccc1C(=O)N1CCC(c2ccnc3[nH]c(N4CCOCC4)nc23)CC1]

Step 5 was performed following the protocol described in step 8 of Examples 355 and 356 using 2-amino-4-(trifluoromethoxy)benzoic acid to give 12 mg (14% yield) of [2-amino-4-(trifluoromethoxy)phenyl]-[4-(2-morpholino-3H-imidazo[4,5-b]pyridin-7-yl)-1-piperidyl]methanone as a white solid. LC/MS (m/z, M+H, Method 1): calc. 491.2, found 491.2; ¹H NMR (400 MHz, DMSO-d6, 120° C.) δ ppm 1.80-1.97 (m, 4H), 2.98-3.14 (m, 2H), 3.27 (quin, J=7.8 Hz, 1H), 3.52-3.60 (m, 4H), 3.69-3.75 (m, 4H), 4.14 (br d, J=13.1 Hz, 2H), 5.25 (br s, 2H), 6.43-6.51 (m, 1H), 6.66-6.70 (m, 1H), 6.80 (br d, J=5.3 Hz, 1H), 7.13 (d, J=8.4 Hz, 1H), 7.78-7.89 (m, 1H), 10.29-11.80 (m, 1H)

### Example 175: (rac)-[2-amino-4-(trifluoromethoxy)phenyl]-[4-[2-(2-methylmorpholin-2-yl)-3H-imidazo[4,5-b]pyridin-7-yl]-1-piperidyl]methanone

Example 175 was prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2. LC/MS (m/z, M+H): 505

### Example 176: [4-[2-[(2S)-6,6-dimethylmorpholin-2-yl]-3H-imidazo[4,5-b]pyridin-7-yl]-1-piperidyl]-[4-(trifluoromethoxy)phenyl]methanone;hydrochloride

Example 176 was prepared following a procedure similar to that of step 1 of Example 5 using (S)-4-(tert-butoxycarbonyl)-6,6-dimethylmorpholine-2-carboxylic acid and [4-(2,3-diamino-4-pyridyl)-1-piperidyl]-[4-(trifluoromethoxy)phenyl]methanone, followed by a procedure similar to that of step 4 of Examples 1 and 2.

### Example 177: [4-[2-[(2R)-6,6-dimethylmorpholin-2-yl]-3H-imidazo[4,5-b]pyridin-7-yl]-1-piperidyl]-[4-(trifluoromethoxy)phenyl]methanone;hydrochloride

Example 177 was prepared following a procedure similar to that of step 1 of Example 5 using (R)-4-(tert-butoxycarbonyl)-6,6-dimethylmorpholine-2-carboxylic acid and [4-(2,3-diamino-4-pyridyl)-1-piperidyl]-[4-(trifluoromethoxy)phenyl]methanone, followed by a procedure similar to that of step 4 of Examples 1 and 2.

### Examples 178 and 179: [2-amino-4-(trifluoromethoxy)phenyl]-[4-[2-[tetrahydrofuran-2-yl]-3H-imidazo[4,5-b]pyridin-7-yl]-1-piperidyl]methanone, Isomers 1 and 2

Examples 178 and 179 were prepared following a procedure similar to that of step 1 of Example 5 using tetrahydrofuran-2-carboxylic acid and tert-butyl 4-(2,3-diamino-4-pyridyl)piperidine-1-carboxylate (described in step 2 of Examples 1 and 2), followed by a procedure similar to that of step 3 of Example 29, followed by a procedure similar to that of step 8 of Examples 355 and 356 using tert-butyl 4-(2,3-diamino-4-pyridyl)piperidine-1-carboxylate, followed by chiral separation using Method CS3.

Example 178 (Isomer 1): LC/MS (m/z, M+H): 476; ¹H NMR (400 MHz, DMSO-d6, 120° C.) δ ppm 1.82-2.11 (m, 6H), 2.22-2.39 (m, 2H), 3.05-3.16 (m, 2H), 3.39-3.51 (m, 1H), 3.85 (td, J=7.7, 6.2 Hz, 1H), 3.96-4.05 (m, 1H), 4.16 (br d, J=13.2 Hz, 2H), 5.07 (t, J=6.8 Hz, 1H), 5.26 (br s, 2H), 6.44-6.51 (m, 1H), 6.68 (dd, J=2.2, 1.0 Hz, 1H), 7.02 (d, J=5.0 Hz, 1H), 7.14 (d, J=8.3 Hz, 1H), 8.18 (d, J=5.0 Hz, 1H), 12.20-12.45 (m, 1H)

### Example 180: [4-[2-(7-oxa-4-azaspiro[2.5]octan-6-yl)-3H-imidazo[4,5-b]pyridin-7-yl]-1-piperidyl]-[4-(trifluoromethoxy)phenyl]methanone;hydrochloride

Example 180 was prepared following a procedure similar to that of Example 252.

### Example 181: [2-amino-4-(trifluoromethoxy)phenyl]-[4-[2-[(2S)-6,6-dimethylmorpholin-2-yl]-3H-imidazo[4,5-b]pyridin-7-yl]-1-piperidyl]methanone;hydrochloride

Example 181 was prepared following a procedure similar to that of step 1 of Example 5 using tert-butyl N-[2-[4-(2,3-diamino-4-pyridyl)piperidine-1-carbonyl]-5-(trifluoromethoxy)phenyl]carbamate and (S)-4-(tert-butoxycarbonyl)-6,6-dimethylmorpholine-2-carboxylic acid, followed by a procedure similar to that of step 4 of Examples 1 and 2. LC/MS (m/z, M+H): 519

### Example 182: [2-amino-4-(trifluoromethoxy)phenyl]-[4-[2-[(2R)-6,6-dimethylmorpholin-2-yl]-3H-imidazo[4,5-b]pyridin-7-yl]-1-piperidyl]methanone;hydrochloride

Example 182 was prepared following a procedure similar to that of step 1 of Example 5 using tert-butyl N-[2-[4-(2,3-diamino-4-pyridyl)piperidine-1-carbonyl]-5-(trifluoromethoxy)phenyl]carbamate and (R)-4-(tert-butoxycarbonyl)-6,6-dimethylmorpholine-2-carboxylic acid, followed by a procedure similar to that of step 4 of Examples 1 and 2.

### Example 183: [4-[2-[1-(oxetan-3-yl)-4-piperidyl]-3H-imidazo[4,5-b]pyridin-7-yl]-1-piperidyl]-[4-(trifluoromethoxy)phenyl]methanone

Example 183 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 530

### Example 184: [4-[2-(2-oxabicyclo[2.2.2]octan-4-yl)-3H-imidazo[4,5-b]pyridin-7-yl]-1-piperidyl]-[4-(trifluoromethoxy)phenyl]methanone

Example 184 was prepared following a procedure similar to that of step 1 of Example 252. LC/MS (m/z, M+H): 501; ¹H NMR (400 MHz, DMSO-d6, 120° C.) δ ppm 1.67-1.80 (m, 2H), 1.85-2.00 (m, 4H), 2.01-2.20 (m, 6H), 3.06-3.20 (m, 2H), 3.39-3.51 (m, 1H), 3.77-3.84 (m, 1H), 4.05 (br s, 2H), 4.16 (br d, J=13.1 Hz, 2H), 7.00 (d, J=5.0 Hz, 1H), 7.36 (br d, J=8.7 Hz, 2H), 7.55 (m, 2H), 8.15 (d, J=5.0 Hz, 1H), 11.99-12.47 (m, 1H)

### Example 185: [4-(2-morpholino-3H-imidazo[4,5-b]pyridin-7-yl)-1-piperidyl]-[4-(trifluoromethoxy)phenyl]methanone

Example 185 was prepared by a procedure similar to that of Example 174 using [4-(2,3-diamino-4-pyridyl)-1-piperidyl]-[4-(trifluoromethoxy)phenyl]methanone (described in step 1 of Example 6) in step 1. LC/MS (m/z, M+H): 476; ¹H NMR (400 MHz, DMSO-d6, 120° C.) δ ppm 1.79-1.97 (m, 4H), 3.03-3.15 (m, 2H), 3.24-3.36 (m, 1H), 3.52-3.60 (m, 4H), 3.68-3.77 (m, 4H), 4.14 (br d, J=12.0 Hz, 2H), 6.82 (d, J=5.3 Hz, 1H), 7.33-7.40 (m, 2H), 7.52-7.58 (m, 2H), 7.85 (br d, J=5.3 Hz, 1H), 11.03-11.74 (m, 1H)

### Examples 186 and 187: [2-amino-4-(trifluoromethoxy)phenyl]-[4-[2-[tetrahydrofuran-3-yl]-3H-imidazo[4,5-b]pyridin-7-yl]-1-piperidyl]methanone, Isomers 1 and 2

Examples 186 and 187 were prepared according to Examples 3 and 4.

Example 186 (Isomer 1): LC/MS (m/z, M+H): 476; ¹H NMR (400 MHz, DMSO-d6, 120° C.) δ ppm 1.84-1.99 (m, 4H), 2.30-2.39 (m, 2H), 3.05-3.16 (m, 2H), 3.35-3.47 ((m, 1H), 3.62-3.72 (m, 1H), 3.83 (dt, J=8.3, 7.1 Hz, 1H), 3.90-3.99 (m, 2H), 4.09 (t, J=7.9 Hz, 1H), 4.16 (br d, J=13.3 Hz, 2H), 5.27 (br s, 2H), 6.44-6.51 (m, 1H), 6.67-6.69 (m, 1H), 7.00 (d, J=5.0 Hz, 1H), 7.14 (d, J=8.4 Hz, 1H), 8.15 (d, J=5.0 Hz, 1H), 11.98-12.60 (m, 1H)

Example 187 (Isomer 2): LC/MS (m/z, M+H): 476; ¹H NMR (400 MHz, DMSO-d6 with AcOD, 120° C.) δ ppm 1.91-2.05 (m, 4H), 2.30-2.40 (m, 2H), 3.06-3.17 (m, 2H), 3.38-3.49 (m, 1H), 3.69 (quin, J=7.5 Hz, 1H), 3.79-3.88 (m, 1H), 3.91-4.02 (m, 2H), 4.07-4.13 (m, 1H), 4.19 (br d, J=13.6 Hz, 2H), 6.49 (br d, J=8.3 Hz, 1H), 6.69 (br s, 1H), 7.01 (d, J=5.0 Hz, 1H), 7.15 (d, J=8.3 Hz, 1H), 8.16 (d, J=5.0 Hz, 1H)

### Example 188: [2-amino-4-(trifluoromethoxy)phenyl]-[4-[2-[1-(oxetan-3-yl)-4-piperidyl]-3H-imidazo[4,5-b]pyridin-7-yl]-1-piperidyl]methanone

Example 188 was prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2. LC/MS (m/z, M+H): 545; ¹H NMR (400 MHz, DMSO-d6, 120° C.) δ ppm 1.84-2.10 (m, 8H), 2.80-2.95 (partially hidden m, 5H), 3.05-3.15 (m, 2H), 3.35-3.46 (m, 1H), 3.52 (quin, J=6.5 Hz, 1H), 4.17 (br d, J=13.2 Hz, 2H), 4.48 (t, J=6.5 Hz, 2H), 4.54 (t, J=6.5 Hz, 2H), 5.27 (br s, 2H), 6.47 (br d, J=8.3 Hz, 1H), 6.65-6.71 (m, 1H), 6.98 (d, J=5.0 Hz, 1H), 7.14 (d, J=8.3 Hz, 1H), 8.13 (d, J=5.0 Hz, 1H), 11.77-12.62 (m, 1H)

### Examples 189 and 190

Examples 189 and 190 were prepared following a procedure similar to that of step 5 of Examples 1 and 2.

Example 189: [2-amino-3-fluoro-4-(trifluoromethoxy)phenyl]-[4-(2-tetrahydropyran-4-yl-3H-imidazo[4,5-b]pyridin-7-yl)-1-piperidyl]methanone; LC/MS (m/z, M+H): 508; ¹H NMR (400 MHz, DMSO-d6, CD₃COOD, 100° C.) δ ppm 1.90-2.02 (m, 8H), 3.05-3.26 (m, 3H), 3.36-3.60 (m, 3H), 3.97 (dt, J=11.4, 3.4×2 Hz, 2H), 4.12-4.27 (m, 2H), 6.62-6.72 (m, 1H), 6.96-7.08 (m, 2H), 8.17 (d, J=5.1 Hz, 1H)

Example 190: tert-butyl N-[2-[4-(2-tetrahydropyran-4-yl-3H-imidazo[4,5-b]pyridin-7-yl)piperidine-1-carbonyl]-5-(trifluoromethoxy)phenyl]carbamate; LC/MS (m/z, M+H): 590

### Example 191: [4-[2-[rac-(2R,3S)-3-aminotetrahydrofuran-2-yl]-3H-imidazo[4,5-b]pyridin-7-yl]-1-piperidyl]-[2-amino-4-(trifluoromethoxy)phenyl]methanone;hydrochloride

Example 191 was prepared by a procedure similar to that of Example 168 using rac-(2S,3R)-3-(tert-butoxycarbonylamino)tetrahydrofuran-2-carboxylic acid and tert-butyl N-[2-[4-(2,3-diamino-4-pyridyl)piperidine-1-carbonyl]-5-(trifluoromethoxy)phenyl]carbamate (described in step 2 of Examples 121 and 122).

### Examples 192 and 193: (cis)-[4-(2-tetrahydropyran-4-yl-3H-imidazo[4,5-b]pyridin-7-yl)cyclohexyl]-[4-(trifluoromethoxy)-1-piperidyl]methanone and (trans)-[4-(2-tetrahydropyran-4-yl-3H-imidazo[4,5-b]pyridin-7-yl)cyclohexyl]-[4-(trifluoromethoxy)-1-piperidyl]methanone

Examples 192 and 193 were prepared following a procedure similar to that of step 5 of Examples 1 and 2.

Example 192 (cis-isomer): LC/MS (m/z, M+H): 481

### Example 194: (rac)-[4-[2-(4-amino-1-methyl-2-oxabicyclo[2.1.1]hexan-3-yl)-3H-imidazo[4,5-b]pyridin-7-yl]-1-piperidyl]-[2-amino-4-(trifluoromethoxy)phenyl]methanone hydrochloride

Example 194 was prepared following a procedure similar to that of step 1 of Example 5 using tert-butyl N-[2-[4-(2,3-diamino-4-pyridyl)piperidine-1-carbonyl]-5-(trifluoromethoxy)phenyl]carbamate (described in step 2 of Examples 121 and 122) and 4-([(tert-butoxy)carbonyl]amino)-1-methyl-2-oxabicyclo[2.1.1]hexane-3-carboxylic acid, followed by a procedure similar to that of step 7 of Examples 355 and 356.

### Example 195: (trans)-[4-[2-(3-methoxycyclobutyl)-3H-imidazo[4,5-b]pyridin-7-yl]-1-piperidyl]-[4-(trifluoromethoxy)phenyl]methanone

Example 195 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 475

### Examples 196 and 197

Examples 196 and 197 were prepared following a procedure similar to that of Example 252.

Example 196: (rac)-[2-amino-4-(trifluoromethoxy)phenyl]-[4-[2-(7-oxa-4-azaspiro[2.5]octan-6-yl)-3H-imidazo[4,5-b]pyridin-7-yl]-1-piperidyl]methanone; LC/MS (m/z, M+H): 517

Example 197: [2-amino-4-(trifluoromethoxy)phenyl]-[4-[2-(2-oxabicyclo[2.2.2]octan-4-yl)-3H-imidazo[4,5-b]pyridin-7-yl]-1-piperidyl]methanone; LC/MS (m/z, M+H): 516; ¹H NMR (400 MHz, DMSO-d6, 101° C.) 80/20 mixture of tautomers, δ ppm 1.65-1.82 (m, 2H), 1.85-1.99 (m, 4H), 2.00-2.20 (m, 6H), 3.03-3.16 (m, 2H), 3.36-3.48 (m, 1H), 3.81 (m, 1H), 4.04 (s, 1.6H), 4.06 (br s, 0.4H), 4.11-4.24 (m, 2H), 5.33 (br s, 1.6H), 5.36 (br s, 0.4H), 6.48 (br d, J=8.3 Hz, 1H), 6.66-6.71 (m, 1H), 6.97-7.04 (m, 1H), 7.11-7.19 (m, 1H), 8.13 (d, J=5.1 Hz, 0.8H), 8.23 (br d, J=5.1 Hz, 0.2H), 11.58-12.82 (m, 1H)

### Example 198: [4-(2-cyclopent-3-en-1-yl-3H-imidazo[4,5-b]pyridin-7-yl)-1-piperidyl]-[4-(trifluoromethoxy)phenyl]methanone

Example 198 was prepared following a procedure similar to that of step 1 of Example 5 using 3-cyclopentene-1-carboxylic acid and [4-(2,3-diamino-4-pyridyl)-1-piperidyl]-[4-(trifluoromethoxy)phenyl]methanone (described in step 1 of Example 6). LC/MS (m/z, M+H): 457; ¹H NMR (400 MHz, DMSO-d6, 120° C.) δ ppm 1.89-2.01 (m, 4H), 2.82 (partially hidden m, 4H), 3.04-3.20 (m, 2H), 3.37-3.58 (m, 1H), 3.71 (quin, J=8.3 Hz, 1H), 4.16 br d, J=11.0 Hz, 2H), 5.77 (br s, 2H), 6.99 (d, J=5.1 Hz, 1H), 7.26-7.44 (m, 2H), 7.51-7.66 (m, 2H), 8.05-8.29 (m, 1H), 11.81-12.46 (m, 1H)

### Example 199: [2-amino-4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone

Example 199 was prepared following a procedure similar to that of step 1 of Example 15 using tert-butyl N-[2-[4-(2,3-diamino-4-pyridyl)piperidine-1-carbonyl]-5-(trifluoromethoxy)phenyl]carbamate with the corresponding carboxylic acid, followed by a procedure similar to that of step 4 of Examples 1 and 2. LC/MS (m/z, M+H): 540; ¹H NMR (400 MHz, DMSO-d6, 120° C.) δ ppm 1.80-2.00 (m, 4H), 2.45 (partially hidden s, 6H), 3.05-3.18 (m, 2H), 3.39-3.50 (m, 1H), 4.16 (br d, J=13.4 Hz, 2H), 5.26 (br s, 2H), 6.45-6.51 (m, 1H), 6.66-6.72 (m, 1H), 7.03 (d, J=5.0 Hz, 1H), 7.14 (d, J=8.3 Hz, 1H), 8.19 (d, J=5.0 Hz, 1H), 11.98-13.06 (m, 1H)

### Example 200: (2-amino-4-bromo-phenyl)-[4-(2-tetrahydropyran-4-yl-3H-imidazo[4,5-b]pyridin-7-yl)-1-piperidyl]methanone

Example 200 was prepared following a procedure similar to that of Example 131 using 2-amino-4-bromobenzoic acid. LC/MS (m/z, M+H): 484

### Example 201: (rac)-[4-[2-(4-amino-1-methyl-2-oxabicyclo[2.1.1]hexan-3-yl)-3H-imidazo[4,5-b]pyridin-7-yl]-1-piperidyl]-[4-(trifluoromethoxy)phenyl]methanone;hydrochloride

Example 201 was prepared following a procedure similar to that of step 1 of Example 5 using [4-(2,3-diamino-4-pyridyl)-1-piperidyl]-[4-(trifluoromethoxy)phenyl]methanone (described in step 1 of Example 6) and 4-([(tert-butoxy)carbonyl]amino)-1-methyl-2-oxabicyclo[2.1.1]hexane-3-carboxylic acid, followed by a procedure similar to that of step 7 of Examples 355 and 356.

### Example 202: N-[2-[4-(2-tetrahydropyran-4-yl-3H-imidazo[4,5-b]pyridin-7-yl)piperidine-1-carbonyl]-5-(trifluoromethoxy)phenyl]acetamide

Example 202 was prepared following a procedure similar to that of step 5 of Examples 1 and 2. LC/MS (m/z, M+H): 532

### Example 203: (trans)-[4-[2-(3-methoxycyclobutyl)-3H-imidazo[4,5-b]pyridin-7-yl]-1-piperidyl]-[4-(trifluoromethoxy)phenyl]methanone

Example 203 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid.

### Examples 204-229

Examples 204-229 were prepared following a procedure similar to that of steps 2 and 3 of Example 6.

### Example 204: [4-[2-(1,2,3,6-tetrahydropyridin-5-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 472.2; ¹H NMR (600 MHz, DMSO-d6) δ ppm 9.02 (br s, 2H), 8.29 (d, J=5.0 Hz, 1H), 7.62-7.59 (m, 2H), 7.47 (br d, J=8.1 Hz, 2H), 7.20 (br d, J=4.8 Hz, 1H), 7.05 (br s, 1H), 4.68 (br s, 1H), 4.18 (br s, 2H), 3.51-3.45 (m, 1H), 3.34-3.22 (m, 2H), 2.96 (br s, 1H), 2.62-2.56 (m, 2H), 2.03-1.79 (m, 4H)

Example 205: [4-[2-[(3R,4S)-4-phenylpyrrolidin-3-yl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 536.2

Example 206: [4-[2-[(2R,4S)-4-fluoropyrrolidin-2-yl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 478.2

Example 207: (rac)-[4-[2-(2-amino-3,3,3-trifluoro-propyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 502.2

Example 208: [4-[2-[6-(aminomethyl)-3-pyridyl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 497.2

Example 209: [4-[2-[rac-(3R,4S)-4-(4-pyridyl)pyrrolidin-3-yl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 537.3

Example 210: [4-[2-(3-amino-2-bicyclo[2.2.1]hept-5-enyl)-3H-imidazo[4,5-b]pyridin-7-yl]-1-piperidyl]-[4-(trifluoromethoxy)phenyl]methanone;2,2,2-trifluoroacetic acid Example 210 was prepared from Boc-3-endo-aminobicyclo[2.2.1]-hept-5-ene-2-endo-carboxylic acid; LC/MS (m/z, M+H): 498.2

Example 211: [4-[2-[(2S)-pyrrolidin-2-yl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 460.2

Example 212: [4-[2-(3-amino-2-thienyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 488.2

Example 213: 4-[7-[1-[4-(trifluoromethoxy)benzoyl]-4-piperidyl]-3H-imidazo[4,5-b]pyridin-2-yl]piperidine-4-carbonitrile;2,2,2-trifluoroacetic acid; LC/MS (m/z, M+H): 499.2

Example 214: [4-[2-[(2R)-2-piperidyl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 474.2

Example 215: [4-[2-(4-aminotetrahydropyran-4-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 490.2

Example 216: [4-[2-(3-amino-4-methoxy-phenyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 512.2

Example 217: (rac)-[4-[2-(2-piperidyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 474.2

Example 218: [4-[2-[(2R)-pyrrolidin-2-yl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 460.2

Example 219: [4-[2-(1,2,3,4-tetrahydroisoquinolin-6-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 522.3

Example 220: [4-[2-(3-amino-5-hydroxy-phenyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 498.2

Example 221: [4-[2-(1H-indol-3-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 506.2

Example 222: [4-[2-(4,5,6,7-tetrahydrothieno[3,2-c]pyridin-2-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 528.2; ¹H NMR (600 MHz, DMSO-d6) δ ppm 9.08 (br s, 2H), 8.27 (br s, 1H), 7.71 (br s, 1H), 7.63-7.60 (m, 2H), 7.47 (br d, J=8.1 Hz, 2H), 7.20 (br s, 1H), 4.68 (br s, 1H), 4.30 (br s, 2H), 3.68 (br s, 1H), 3.50-3.47 (m, 4H), 3.16-3.10 (m, 2H), 2.99 (br s, 1H), 2.05-1.82 (m, 4H)

Example 223: (rac)-[4-[2-(azetidin-2-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 446.2

Example 224: (rac)-[4-[2-(3-oxa-9-azabicyclo[3.3.1]nonan-7-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 516.3

Example 225: [4-[2-(4-amino-3-pyridyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 483.2

Example 226: [4-[2-[(2R)-piperazin-2-yl]-3H-imidazo[4,5-b]pyridin-7-yl]-1-piperidyl]-[4-(trifluoromethoxy)phenyl]methanone;2,2,2-trifluoroacetic acid

Example 227: [4-[2-(3-aminocyclobutyl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 460.2

Example 228: [4-[2-[3-(methylamino)phenyl]-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 496.2

Example 229: (rac)-[4-[2-(2-azabicyclo[4.1.0]heptan-7-yl)-3H-imidazo[4,5-b]pyridin-7-yl]-1-piperidyl]-[4-(trifluoromethoxy)phenyl]methanone;2,2,2-trifluoroacetic acid; LC/MS (m/z, M+H): 486.2

### Examples 230-236

Examples 230-236 were prepared following a procedure similar to that of step 2 of Example 6.

Example 230: [4-[2-[4-hydroxy-6-(trifluoromethyl)-3-quinolyl]-3H-imidazo[4,5-b]pyridin-7-yl]-1-piperidyl]-[4-(trifluoromethoxy)phenyl]methanone; LC/MS (m/z, M+H): 602.2

Example 231: (rac)-[4-[2-(3-oxabicyclo[3.1.0]hexan-6-yl)-3H-imidazo[4,5-b]pyridin-7-yl]-1-piperidyl]-[4-(trifluoromethoxy)phenyl]methanone; LC/MS (m/z, M+H): 473.2

Example 232: N-[4-hydroxy-2-[7-[1-[4-(trifluoromethoxy)benzoyl]-4-piperidyl]-3H-imidazo[4,5-b]pyridin-2-yl]phenyl]acetamide; LC/MS (m/z, M+H): 540.2

Example 233: 5,6-dimethyl-3-[7-[1-[4-(trifluoromethoxy)benzoyl]-4-piperidyl]-3H-imidazo[4,5-b]pyridin-2-yl]-1H-pyridin-2-one; LC/MS (m/z, M+H): 512.2

Example 234: 1-[3,5-dimethyl-4-[7-[1-[4-(trifluoromethoxy)benzoyl]-4-piperidyl]-3H-imidazo[4,5-b]pyridin-2-yl]-1H-pyrrol-2-yl]ethanone; LC/MS (m/z, M+H): 526.2

Example 235: [4-[2-[1-(1,1-dioxothiolan-3-yl)-4-piperidyl]-3H-imidazo[4,5-b]pyridin-7-yl]-1-piperidyl]-[4-(trifluoromethoxy)phenyl]methanone; LC/MS (m/z, M+H): 592.2

Example 236: [4-[2-(1-methylsulfonylazetidin-3-yl)-3H-imidazo[4,5-b]pyridin-7-yl]-1-piperidyl]-[4-(trifluoromethoxy)phenyl]methanone; LC/MS (m/z, M+H): 524.2

### Example 237: [2-hydroxy-4-(trifluoromethoxy)phenyl]-[4-(2-tetrahydropyran-4-yl-3H-imidazo[4,5-b]pyridin-7-yl)-1-piperidyl]methanone

Example 237 was prepared following a procedure similar to that of Example 131 using 2-hydroxy-4-(trifluoromethoxy)benzoic acid. LC/MS (m/z, M+H): 491

### Example 238: (rac)-[4-[2-(1,4-oxazepan-2-yl)-3H-imidazo[4,5-b]pyridin-7-yl]-1-piperidyl]-[4-(trifluoromethoxy)phenyl]methanone;hydrochloride

Example 238 was prepared by a procedure similar to that of step 1 of Example 5 using 4-Boc-2-homomorpholinecarboxylic acid and [4-(2,3-diamino-4-pyridyl)-1-piperidyl]-[4-(trifluoromethoxy)phenyl]methanone (described in step 1 of Example 6), followed by a procedure similar to that of step 7 of Examples 355 and 356. LC/MS (m/z, M+H): 490

### Example 239: [4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone

Example 239 was prepared following a procedure similar to that of step 1 of Example 15 using [4-(2,3-diamino-4-pyridyl)-1-piperidyl]-[4-(trifluoromethoxy)phenyl]methanone and the corresponding carboxylic acid.

### Examples 240 and 241 [4-(1,1,2,2-tetrafluoroethoxy)phenyl]-[4-(2-tetrahydropyran-4-yl-3H-imidazo[4,5-b]pyridin-7-yl)-1-piperidyl]methanone and [4-(1,1,2,2,2-pentafluoroethoxy)phenyl]-[4-(2-tetrahydropyran-4-yl-3H-imidazo[4,5-b]pyridin-7-yl)-1-piperidyl]methanone

Examples 240 and 241 were prepared following a procedure similar to that of step 5 of Examples 1 and 2.

### Example 242: [4-(2-phenyl-3H-imidazo[4,5-b]pyridin-7-yl)-1-piperidyl]-[4-(trifluoromethoxy)phenyl]methanone

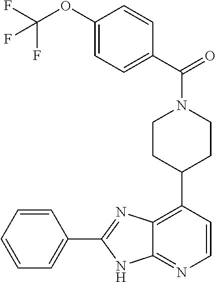 [SMILES: O=C(c1ccc(OC(F)(F)F)cc1)N1CCC(c2ccnc3[nH]c(-c4ccccc4)nc23)CC1]

To a solution of [4-(3H-imidazo[4,5-b]pyridin-7-yl)-1-piperidyl]-[4-(trifluoromethoxy)phenyl]methanone (described in example 18, 100 mg, 0.26 mmol) in Eaton's reagent (1 mL) was added benzoic acid (34 mg, 0.28 mmol) and the resulting reaction mixture was stirred at 125° C. for one hour, then cooled down to room temperature, and poured onto crushed ice. Then, solid Na₂CO₃ was slowly added to pH 7-8, diluted with water, and the whole was extracted twice with AcOEt. The combined organic layers were dried over magnesium sulfate, filtered and concentrated. The resulting residue was purified on silica gel (SiO₂ 20 g) eluting with DCM/(DCM/MeOH 90/10) 70/30 to give 72 mg (60% yield) of [4-(2-phenyl-3H-imidazo[4,5-b]pyridin-7-yl)-1-piperidyl]-[4-(trifluoromethoxy)phenyl]methanone as a white solid. LC/MS (m/z, M+H, Method 1): calc. 467.2, found 467.1.

### Example 243: (2,2,3,3-tetrafluoro-1,4-benzodioxin-6-yl)-[4-(2-tetrahydropyran-4-yl-3H-imidazo[4,5-b]pyridin-7-yl)-1-piperidyl]methanone

Example 243 was prepared following a procedure similar to that of step 5 of Examples 1 and 2.

### Example 244: [4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone

Example 244 was prepared following a procedure similar to that of step 1 of Example 15 using [4-(2,3-diamino-4-pyridyl)-1-piperidyl]-[4-(trifluoromethoxy)phenyl]methanone and the corresponding carboxylic acid. LC/MS (m/z, M+H): 557

### Examples 245 and 246: [2-amino-4-(trifluoromethoxy)phenyl]-[4-[2-[7-oxa-4-azaspiro[2.5]octan-6-yl]-3H-imidazo[4,5-b]pyridin-7-yl]-1-piperidyl]methanone, Isomers 1 and 2

Examples 245 and 246 were prepared following a procedure similar to that of Example 252, followed by chiral separation using Method CS10.

### Example 247: [4-[2-[rac-(1S,3S)-3-aminocyclohexyl]-3H-imidazo[4,5-b]pyridin-7-yl]-1-piperidyl]-[4-(trifluoromethoxy)phenyl]methanone

Example 247 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid, followed by a procedure similar to that of step 4 of Examples 1 and 2. LC/MS (m/z, M+H): 488

### Example 248: [4-(2-cyclopentyl-3H-imidazo[4,5-b]pyridin-7-yl)-1-piperidyl]-[4-(trifluoromethoxy)phenyl]methanone

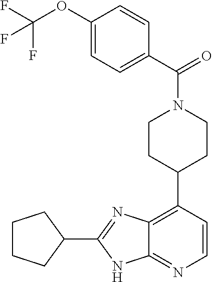 [SMILES: O=C(c1ccc(OC(F)(F)F)cc1)N1CCC(c2ccnc3[nH]c(C4CCCC4)nc23)CC1]

To a solution of [4-(2-cyclopent-3-en-1-yl-3H-imidazo[4,5-b]pyridin-7-yl)-1-piperidyl]-[4-(trifluoromethoxy)phenyl]methanone (Example 198; 60 mg, 0.13 mmol) in MeOH (6 mL), was added Pd/C (14 mg, 0.013 mmol) and the mixture was stirred under H₂ atmosphere (2 bars) at 25° C. for 12 hours. 6 mg of additional Pd/C was added and the mixture was stirred under H₂ atmosphere (2 bars) at 25° C. for 3.5 hours. The mixture was filtered through celite, and the solvent was concentrated under vacuum. The resulting residue was purified on silica gel (SiO₂ 10 g) eluting with DCM/iPrOH 95/5 to give 43 mg (71% yield) of [4-(2-cyclopentyl-3H-imidazo[4,5-b]pyridin-7-yl)-1-piperidyl]-[4-(trifluoromethoxy)phenyl]methanone as a white solid. LC/MS (m/z, M+H, Method 1): calc. 459.2, found 459.2.

### Example 249: [4-[2-(4-methylpiperazin-1-yl)-3H-imidazo[4,5-b]pyridin-7-yl]-1-piperidyl]-[4-(trifluoromethoxy)phenyl]methanone;hydrochloride

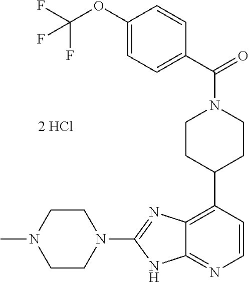 [SMILES: CN1CCN(c2nc3c(C4CCN(C(=O)c5ccc(OC(F)(F)F)cc5)CC4)ccnc3[nH]2)CC1]

### Step 1: 2-Bromo-7-(4-piperidyl)-3H-imidazo[4,5-b]pyridine;hydrobromide

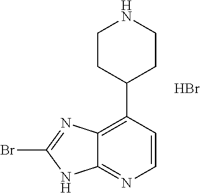 [SMILES: Brc1nc2c(C3CCNCC3)ccnc2[nH]1 ; Br]

To a solution of tert-butyl 4-(2-thioxo-1,3-dihydroimidazo[4,5-b]pyridin-7-yl)piperidine-1-carboxylate (500 mg, 1.49 mmol) in THF (5 mL) was added, at −50° C., 2.5 mL of acetic acid and hydrobromic acid (500 μL, 9.73 mmol). The resulting was stirred at −50° C. for 30 minutes. The reaction mixture was then diluted with diethyl ether, filtered, and washed with diethyl ether to give 180 mg (33% yield) of crude 2-bromo-7-(4-piperidyl)-3H-imidazo[4,5-b]pyridine;hydrobromide as a yellow solid which was used in the next step without further purification.

### Step 2: 2-(4-Methylpiperazin-1-yl)-7-(4-piperidyl)-3H-imidazo[4,5-b]pyridine;hydrobromide

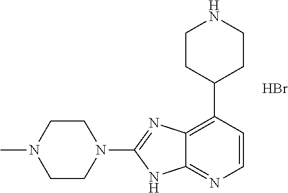 [SMILES: CN1CCN(c2nc3c(C4CCNCC4)ccnc3[nH]2)CC1 ; Br]

In a microwave vial, to 2-bromo-7-(4-piperidyl)-3H-imidazo[4,5-b]pyridine;hydrobromide (130 mg, 0.36 mmol) was added 1-methylpiperazine (3488 mg, 34.83 mmol) and DMF (0.5 mL). The vial was sealed and submitted to microwave at 190° C., for 30 minutes followed by 20 minutes at 220° C. Then, the reaction mixture was concentrated in vacuo. The resulting residue was diluted with DCM, transferred to a separating funnel, and washed with water. The aqueous layer was extracted with DCM, and the combined organic layers were dried over magnesium sulfate, filtered and concentrated in vacuo. The resulting residue was triturated in diethyl ether to give 50 mg (36% yield) of crude 2-(4-methylpiperazin-1-yl)-7-(4-piperidyl)-3H-imidazo[4,5-b]pyridine;hydrobromide as a pale brown solid which was used in the next step without further purification.

### Step 3: [4-[2-(4-Methylpiperazin-1-yl)-3H-imidazo[4,5-b]pyridin-7-yl]-1-piperidyl]-[4-(trifluoromethoxy)phenyl]methanone;hydrochloride

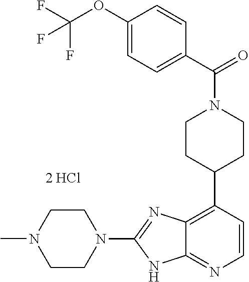 [SMILES: CN1CCN(c2nc3c(C4CCN(C(=O)c5ccc(OC(F)(F)F)cc5)CC4)ccnc3[nH]2)CC1]

Step 3 was performed following the protocol described in step 8 of Examples 355 and 356 using 4-(trifluoromethoxy)benzoic acid. The obtained residue was suspended with a 2 N HCl solution in Et₂O, and filtered to give 18 mg (25% yield) of [4-[2-(4-methylpiperazin-1-yl)-3H-imidazo[4,5-b]pyridin-7-yl]-1-piperidyl]-[4-(trifluoromethoxy)phenyl]methanone;hydrochloride as a white solid. LC/MS (m/z, M+H-2 HCl, Method 1): calc. 489.2, found 489.2.

### Example 250: [4-[2-[7-oxa-4-azaspiro[2.5]octan-6-yl]-3H-imidazo[4,5-b]pyridin-7-yl]-1-piperidyl]-[4-(trifluoromethoxy)phenyl]methanone, Isomer 1

Example 250 was obtained by chiral separation of the compound of Example 180 using Method CS4; Isomer 1 was the first eluting isomer.

### Example 251: [2-amino-4-(trifluoromethoxy)phenyl]-[4-(2-cyclopent-3-en-1-yl-3H-imidazo[4,5-b]pyridin-7-yl)-1-piperidyl]methanone;hydrochloride

Example 251 was prepared following a procedure similar to that of step 1 of Example 5 using 3-cyclopentene-1-carboxylic acid and tert-butyl N-[2-[4-(2,3-diamino-4-pyridyl)piperidine-1-carbonyl]-5-(trifluoromethoxy)phenyl]carbamate (described in step 2 of Examples 121 and 122), followed by a procedure similar to that of step 7 of Examples 355 and 356. LC/MS (m/z, M+H): 472

### Example 252 (rac)-[4-[2-(Azepan-3-yl)-3H-imidazo[4,5-b]pyridin-7-yl]-1-piperidyl]-[4-(trifluoromethoxy)phenyl]methanone

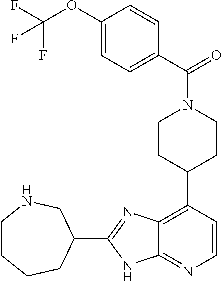 [SMILES: O=C(c1ccc(OC(F)(F)F)cc1)N1CCC(c2ccnc3[nH]c(C4CCCCNC4)nc23)CC1]

### Step 1: (rac)-tert-Butyl 3-[7-[1-[4-(trifluoromethoxy)benzoyl]-4-piperidyl]-3H-imidazo[4,5-b]pyridin-2-yl]azepane-1-carboxylate

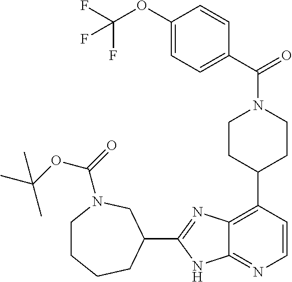 [SMILES: CC(C)(C)OC(=O)N1CCCCC(c2nc3c(C4CCN(C(=O)c5ccc(OC(F)(F)F)cc5)CC4)ccnc3[nH]2)C1]

In a 10-20 mL microwave vial, were placed (rac)-1-tert-butoxycarbonylazepane-3-carboxylic acid (96 mg, 0.39 mmol), CDI (64 mg, 0.39 mmol) and acetonitrile (10 mL). The vial was sealed and submitted to microwave at 120° C. for 15 minutes. Then, 1-tert-butoxycarbonylazepane-3-carboxylic acid (100 mg, 0.39 mmol) was added to the mixture, which was then submitted to microwave at 140° C. for 90 minutes. After 90 minutes, the reaction mixture was cooled down to room temperature, diluted with ethyl acetate and water, and extracted twice with ethyl acetate. The organic layers were combined, dried over magnesium sulfate, filtered, and concentrated to dryness. The resulting residue was then purified on silica gel (SiO₂ 10 g) eluting with DCM/MeOH/NH₄OH 90/10/2 to give 60 mg (39% yield) of (rac)-tert-butyl 3-[7-[1-[4-(trifluoromethoxy)benzoyl]-4-piperidyl]-3H-imidazo[4,5-b]pyridin-2-yl]azepane-1-carboxylate as a white solid. LC/MS (m/z, M+H): 588.

### Step 2: (rac)-[4-[2-(Azepan-3-yl)-3H-imidazo[4,5-b]pyridin-7-yl]-1-piperidyl]-[4-(trifluoromethoxy)phenyl]methanone

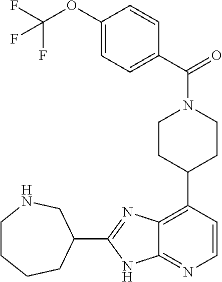 [SMILES: O=C(c1ccc(OC(F)(F)F)cc1)N1CCC(c2ccnc3[nH]c(C4CCCCNC4)nc23)CC1]

A solution of (rac)-tert-butyl 3-[7-[1-[4-(trifluoromethoxy)benzoyl]-4-piperidyl]-3H-imidazo[4,5-b]pyridin-2-yl]azepane-1-carboxylate (60 mg, 0.1 mmol) in ethyl acetate (2 mL) was cooled down to 0° C., and then 0.61 mL of a 2 M HCl solution was added. The resulting reaction mixture was then stirred at room temperature for 12 hours. After 12 hours, the reaction mixture was concentrated to dryness and the resulting residue was purified by flash chromatography on silica gel (SiO₂ 10 g) eluting with DCM/MeOH/NH₄OH 90/10/2 to give 49 mg (100% yield) of (rac)-[4-[2-(azepan-3-yl)-3H-imidazo[4,5-b]pyridin-7-yl]-1-piperidyl]-[4-(trifluoromethoxy)phenyl]methanone as a white solid. LC/MS (m/z, M+H, Method 1): calc. 488.2, found 488.2.

### Examples 253: [2-amino-4-(trifluoromethoxy)phenyl]-[4-(2-cyclopentyl-3H-imidazo[4,5-b]pyridin-7-yl)-1-piperidyl]methanone;hydrochloride

Example 253 was prepared following a procedure similar to that of step 1 of Example 5 using 3-cyclopentene-1-carboxylic acid and tert-butyl N-[2-[4-(2,3-diamino-4-pyridyl)piperidine-1-carbonyl]-5-(trifluoromethoxy)phenyl]carbamate (described in step 2 of Examples 121 and 122), followed by a procedure similar to that of Example 98, followed by a procedure similar to that of step 4 of Examples 1 and 2. LC/MS (m/z, M+H): 474

### Examples 254 and 255: [4-[2-[4-amino-1-methyl-2-oxabicyclo[2.1.1]hexan-3-yl]-3H-imidazo[4,5-b]pyridin-7-yl]-1-piperidyl]-[2-amino-4-(trifluoromethoxy)phenyl]methanone, Isomers 1 and 2

Examples 254 and 255 were prepared by a procedure similar to that of step 1 of Example 5, using 4-([(tert-butoxy)carbonyl]amino)-1-methyl-2-oxabicyclo[2.1.1]hexane-3-carboxylic acid and tert-butyl N-[2-[4-(2,3-diamino-4-pyridyl)piperidine-1-carbonyl]-5-(trifluoromethoxy)phenyl]carbamate (described in step 2 of Examples 121 and 122), followed by a procedure similar to that of step 7 of Examples 355 and 356, followed by chiral separation using Method CS2.

### Example 256: (rac)-[2-amino-4-(trifluoromethoxy)phenyl]-[4-[2-(3-piperidyl)-3H-imidazo[4,5-b]pyridin-7-yl]-1-piperidyl]methanone

Example 256 was prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to that of step 4 of Examples 1 and 2. LC/MS (m/z, M+H): 489

### Example 257: [4-[2-(3-hydroxyphenyl)-3H-imidazo[4,5-b]pyridin-7-yl]-1-piperidyl]-[4-(trifluoromethoxy)phenyl]methanone

Example 257 was prepared following a procedure similar to that of step 1 of Example 20 using [4-(2,3-diamino-4-pyridyl)-1-piperidyl]-[4-(trifluoromethoxy)phenyl]methanone and the corresponding carboxylic acid. LC/MS (m/z, M+H): 483; ¹H NMR (400 MHz, DMSO-d6, 120° C.) δ ppm 1.80-2.10 (m, 4H), 3.07-3.25 (m, 2H), 3.44-3.62 (m, 1H), 4.19 (br d, J=10.8 Hz, 2H), 6.91 (br dd, J=7.9, 2.1 Hz, 1H), 7.07 (br d, J=5.1 Hz, 1H), 7.31 (br t, J=7.9 Hz, 1H), 7.38 (br d, J=8.4 Hz, 2H), 7.58 (br d, J=8.4 Hz, 2H), 7.61-7.68 (m, 2H), 8.22 (br d, J=5.1 Hz, 1H), 8.99-9.53 (m, 1H), 12.47-13.30 (m, 1H)

### Example 258: [4-[2-[rac-(1R,3S)-3-aminocyclohexyl]-3H-imidazo[4,5-b]pyridin-7-yl]-1-piperidyl]-[4-(trifluoromethoxy)phenyl]methanone

Example 258 was prepared following a procedure similar to that of step 1 of Example 20 using tert-butyl N-[2-[4-(2,3-diamino-4-pyridyl)piperidine-1-carbonyl]-5-(trifluoromethoxy)phenyl]carbamate and the corresponding carboxylic acid. LC/MS (m/z, M+H): 488

### Example 259: (rac)-[4-(2-tetrahydropyran-4-yl-3H-imidazo[4,5-b]pyridin-7-yl)azepan-1-yl]-[4-(trifluoromethoxy)phenyl]methanone

Example 259 was prepared following a procedure similar to that of Examples 332 and 333 using tert-butyl 4-(4,4,5,5-tetramethyl-1,3,2-dioxaborolan-2-yl)-2,3,6,7-tetrahydro-1H-azepine-1-carboxylate in step 1, and 4-(trifluoromethoxy)benzoic acid in step 4.

### Example 260: (rac)-[2-amino-4-(trifluoromethoxy)phenyl]-[4-(2-tetrahydropyran-4-yl-3H-imidazo[4,5-b]pyridin-7-yl)azepan-1-yl]methanone

Example 260 was prepared following a procedure similar to that of Examples 332 and 333 using tert-butyl 4-(4,4,5,5-tetramethyl-1,3,2-dioxaborolan-2-yl)-2,3,6,7-tetrahydro-1H-azepine-1-carboxylate in step 1, and 2-amino-4-(trifluoromethoxy)benzoic acid in step 4.

### Examples 261 and 262: (rac)-[4-[2-(3-fluoro-4-piperidyl)-3H-imidazo[4,5-b]pyridin-7-yl]-1-piperidyl]-[4-(trifluoromethoxy)phenyl]methanone and [4-[2-(1,2,3,6-tetrahydropyridin-4-yl)-3H-imidazo[4,5-b]pyridin-7-yl]-1-piperidyl]-[4-(trifluoromethoxy)phenyl]methanone

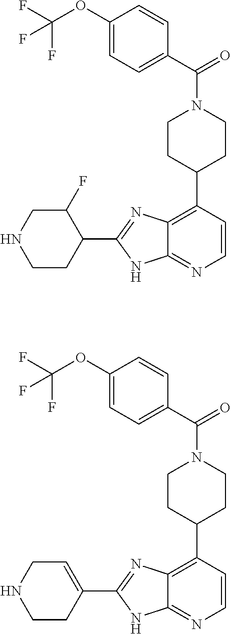 [SMILES: O=C(c1ccc(OC(F)(F)F)cc1)N1CCC(c2ccnc3[nH]c(C4CCNCC4F)nc23)CC1 ; O=C(c1ccc(OC(F)(F)F)cc1)N1CCC(c2ccnc3[nH]c(C4=CCNCC4)nc23)CC1]

### Step 1: tert-butyl (rac)-3-fluoro-4-[7-[1-[4-(trifluoromethoxy)benzoyl]-4-piperidyl]-3H-imidazo[4,5-b]pyridin-2-yl]piperidine-1-carboxylate & tert-butyl 4-[7-[1-[4-(trifluoromethoxy)benzoyl]-4-piperidyl]-3H-imidazo[4,5-b]pyridin-2-yl]-3,6-dihydro-2H-pyridine-1-carboxylate

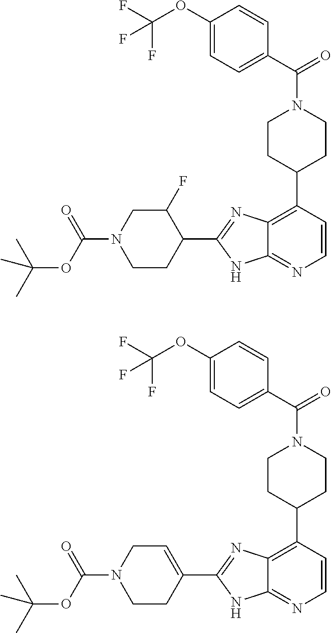 [SMILES: CC(C)(C)OC(=O)N1CCC(c2nc3c(C4CCN(C(=O)c5ccc(OC(F)(F)F)cc5)CC4)ccnc3[nH]2)C(F)C1 ; CC(C)(C)OC(=O)N1CC=C(c2nc3c(C4CCN(C(=O)c5ccc(OC(F)(F)F)cc5)CC4)ccnc3[nH]2)CC1]

Step 1 was performed following a modified protocol of step 1 of Example 5 (the reaction was run under reflux for 6.5 hours instead of microwave irradiation, and after 6.5 hours, 0.5 mL of acetic was added and the reaction was refluxed for 30 minutes) using [4-(2,3-diamino-4-pyridyl)-1-piperidyl]-[4-(trifluoromethoxy)phenyl]methanone (described in step 1 of Example 6) and 1-(tert-butoxycarbonyl)-3-fluoropiperidine-4-carboxylic acid to give 128 mg (approx. 57% yield) of a mixture of tert-butyl (rac)-3-fluoro-4-[7-[1-[4-(trifluoromethoxy)benzoyl]-4-piperidyl]-3H-imidazo[4,5-b]pyridin-2-yl]piperidine-1-carboxylate & tert-butyl 4-[7-[1-[4-(trifluoromethoxy)benzoyl]-4-piperidyl]-3H-imidazo[4,5-b]pyridin-2-yl]-3,6-dihydro-2H-pyridine-1-carboxylate which was used in the next step.

### Step 2: (rac)-[4-[2-(3-fluoro-4-piperidyl)-3H-imidazo[4,5-b]pyridin-7-yl]-1-piperidyl]-[4-(trifluoromethoxy)phenyl]methanone and [4-[2-(1,2,3,6-tetrahydropyridin-4-yl)-3H-imidazo[4,5-b]pyridin-7-yl]-1-piperidyl]-[4-(trifluoromethoxy)phenyl]methanone

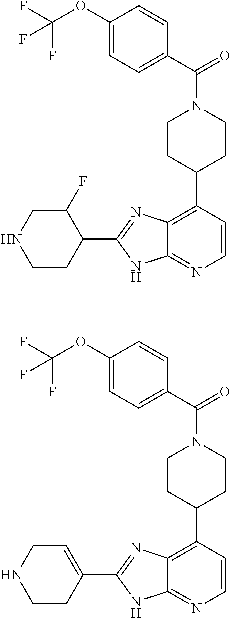 [SMILES: O=C(c1ccc(OC(F)(F)F)cc1)N1CCC(c2ccnc3[nH]c(C4CCNCC4F)nc23)CC1 ; O=C(c1ccc(OC(F)(F)F)cc1)N1CCC(c2ccnc3[nH]c(C4=CCNCC4)nc23)CC1]

Step 2 was performed following the protocol described in step 7 of Examples 355 and 356 (using HCl 4N dioxane) to give both compounds separately.

Example 261: 12 mg (5% yield) of (rac)-[4-[2-(3-fluoro-4-piperidyl)-3H-imidazo[4,5-b]pyridin-7-yl]-1-piperidyl]-[4-(trifluoromethoxy)phenyl]methanone as a white solid. LC/MS (m/z, M+H, Method 1): calc. 492.2, found 492.0.

Example 262: 37 mg (17% yield) of 4-[2-(1,2,3,6-tetrahydropyridin-4-yl)-3H-imidazo[4,5-b]pyridin-7-yl]-1-piperidyl]-[4-(trifluoromethoxy)phenyl]methanone as a white solid. LC/MS (m/z, M+H, Method 1): calc. 472.2, found 472.2.

### Example 263: 3-[7-[1-[4-(trifluoromethoxy)benzoyl]-4-piperidyl]-3H-imidazo[4,5-b]pyridin-2-yl]bicyclo[1.1.1]pentane-1-carboxylic acid

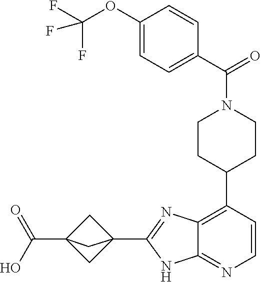 [SMILES: O=C(c1ccc(OC(F)(F)F)cc1)N1CCC(c2ccnc3[nH]c(C45CC(C(=O)O)(C4)C5)nc23)CC1]

### Step 1: Methyl 3-[7-[1-[4-(trifluoromethoxy)benzoyl]-4-piperidyl]-3H-imidazo[4,5-b]pyridin-2-yl]bicyclo[1.1.1]pentane-1-carboxylate

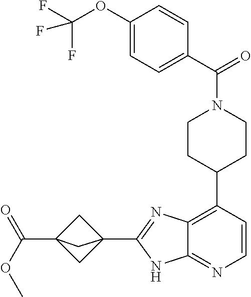 [SMILES: COC(=O)C12CC(c3nc4c(C5CCN(C(=O)c6ccc(OC(F)(F)F)cc6)CC5)ccnc4[nH]3)(C1)C2]

Step 1 was performed following the protocol described in step 1 of Example 20 using [4-(2,3-diamino-4-pyridyl)-1-piperidyl]-[4-(trifluoromethoxy)phenyl]methanone (described in step 1 of Example 6) and 3-(methoxycarbonyl)bicyclo[1.1.1]pentane-1-carboxylic acid to give 171 mg (84% yield) of methyl 3-[7-[1-[4-(trifluoromethoxy)benzoyl]-4-piperidyl]-3H-imidazo[4,5-b]pyridin-2-yl]bicyclo[1.1.1]pentane-1-carboxylate as a white powder.

### Step 2: 3-[7-[1-[4-(trifluoromethoxy)benzoyl]-4-piperidyl]-3H-imidazo[4,5-b]pyridin-2-yl]bicyclo[1.1.1]pentane-1-carboxylic acid

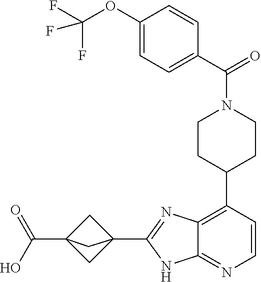 [SMILES: O=C(c1ccc(OC(F)(F)F)cc1)N1CCC(c2ccnc3[nH]c(C45CC(C(=O)O)(C4)C5)nc23)CC1]

To a solution of methyl 3-[7-[1-[4-(trifluoromethoxy)benzoyl]-4-piperidyl]-3H-imidazo[4,5-b]pyridin-2-yl]bicyclo[1.1.1]pentane-1-carboxylate (165 mg, 0.32 mmol) in MeOH (3 mL) was added 0.64 mL of a 2 N solution of NaOH (0.64 mL, 1.28 mmol) and the whole mixture was stirred at room temperature for 12 hours. Then, the reaction mixture was concentrated in vacuo, diluted with water (3 mL), acidified with 4 equivalents of HCl (2 N HCl solution) to pH 5-6. The resulting precipitate was sonicated, filtered, washed with water and dried under vacuum to give 124 mg of a white powder which was then triturated in MeCN, filtered to give 103 mg (64% yield) of 3-[7-[1-[4-(trifluoromethoxy)benzoyl]-4-piperidyl]-3H-imidazo[4,5-b]pyridin-2-yl]bicyclo[1.1.1]pentane-1-carboxylic acid as a white solid. LC/MS (m/z, M+H, Method 1): calc. 501.2, found 501.2.

### Example 264: 3-[4-(2-tetrahydropyran-4-yl-3H-imidazo[4,5-b]pyridin-7-yl)piperidine-1-carbonyl]-6-(trifluoromethyl)-1H-pyridin-2-one

Example 264 was prepared following a procedure similar to that of step 5 of Examples 1 and 2.

### Example 265: [4-(difluoromethoxy)phenyl]-[4-(2-tetrahydropyran-4-yl-3H-imidazo[4,5-b]pyridin-7-yl)-1-piperidyl]methanone

Example 265 was prepared following a procedure similar to that of step 5 of Examples 1 and 2.

### Examples 266 and 267: [2-amino-4-(trifluoromethoxy)phenyl]-[4-[2-[1,1-dioxothiolan-3-yl]-3H-imidazo[4,5-b]pyridin-7-yl]-1-piperidyl]methanone, Isomers 1 and 2

Examples 266 and 267 were prepared following a procedure similar to that of step 1 of Example 15 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to step 4 of Examples 1 and 2, followed by chiral separation using Method CS9.

### Examples 268 and 269: [4-[2-[1,4-oxazepan-2-yl]-3H-imidazo[4,5-b]pyridin-7-yl]-1-piperidyl]-[4-(trifluoromethoxy)phenyl]methanone;hydrochloride, Isomers 1 and 2

Examples 268 and 269 were prepared following a procedure similar to that of step 1 of Example 5 using [2-amino-4-(trifluoromethoxy)phenyl]-[4-(2,3-diamino-4-pyridyl)-1-piperidyl]methanone and the corresponding carboxylic acid, followed by a procedure similar to step 4 of Examples 1 and 2, followed by chiral separation using Method CS10.

Example 268 (Isomer 1): LC/MS (m/z, M+H): 490

### Examples 270 and 271: [2-amino-4-(trifluoromethoxy)phenyl]-[4-[2-[1,4-oxazepan-2-yl]-3H-imidazo[4,5-b]pyridin-7-yl]-1-piperidyl]methanone;hydrochloride, Isomers 1 and 2

Examples 270 and 271 were prepared following a procedure similar to that of step 1 of Example 5 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to step 4 of Examples 1 and 2, followed by chiral separation using Method CS10.

Example 270 (Isomer 1): LC/MS (m/z, M+H): 505

Example 271 (Isomer 2): LC/MS (m/z, M+H): 505

### Examples 272 and 273: [4-(2-tetrahydropyran-4-yl-3H-imidazo[4,5-b]pyridin-7-yl)azepan-1-yl]-[4-(trifluoromethoxy)phenyl]methanone, Isomers 1 and 2

Examples 272 and 273 were obtained by chiral separation of the compound of Example 259 using Method CS3 (n-Heptane/EtOH 80/20).

### Examples 274 and 275: [2-amino-4-(trifluoromethoxy)phenyl]-[4-(2-tetrahydropyran-4-yl-3H-imidazo[4,5-b]pyridin-7-yl)azepan-1-yl]methanone, Isomers 1 and 2

Examples 274 and 275 were obtained by chiral separation of the compound of Example 260 using Method CS3 (flow rate 45 mL/min, n-Heptane/EtOH 80/20).

### Example 276: (trans)-4-[7-[1-[4-(trifluoromethoxy)benzoyl]-4-piperidyl]-3H-imidazo[4,5-b]pyridin-2-yl]cyclohexanecarboxylic acid;hydrochloride

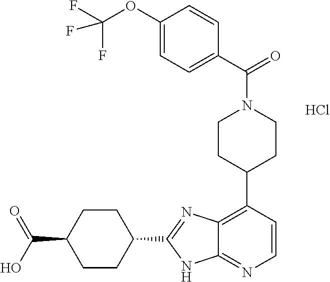 [SMILES: O=C(c1ccc(OC(F)(F)F)cc1)N1CCC(c2ccnc3[nH]c([C@H]4CC[C@H](C(=O)O)CC4)nc23)CC1 ; Cl]

### Step 1: (trans)-methyl 4-[7-[1-[4-(trifluoromethoxy)benzoyl]-4-piperidyl]-3H-imidazo[4,5-b]pyridin-2-yl]cyclohexanecarboxylate

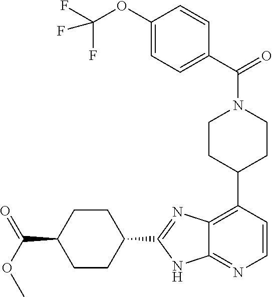 [SMILES: COC(=O)[C@H]1CC[C@H](c2nc3c(C4CCN(C(=O)c5ccc(OC(F)(F)F)cc5)CC4)ccnc3[nH]2)CC1]

Step 1 was performed following the protocol described in step 1 of Example 15 using [4-(2,3-diamino-4-pyridyl)-1-piperidyl]-[4-(trifluoromethoxy)phenyl]methanone (described in step 1 of Example 6) and (trans)-1,4-cyclohexanedicarboxylic acid monomethyl ester to give 219 mg (78% yield) of methyl 4-[7-[1-[4-(trifluoromethoxy)benzoyl]-4-piperidyl]-3H-imidazo[4,5-b]pyridin-2-yl]cyclohexanecarboxylate as a yellow solid.

### Step 2: (trans)-4-[7-[1-[4-(trifluoromethoxy)benzoyl]-4-piperidyl]-3H-imidazo[4,5-b]pyridin-2-yl]cyclohexanecarboxylic acid;hydrochloride

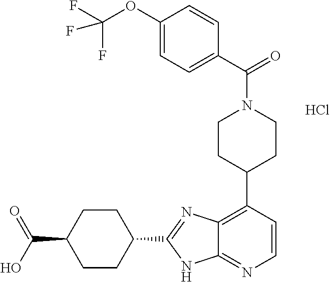 [SMILES: O=C(c1ccc(OC(F)(F)F)cc1)N1CCC(c2ccnc3[nH]c([C@H]4CC[C@H](C(=O)O)CC4)nc23)CC1 ; Cl]

To a solution of methyl 4-[7-[1-[4-(trifluoromethoxy)benzoyl]-4-piperidyl]-3H-imidazo[4,5-b]pyridin-2-yl]cyclohexanecarboxylate (219 mg, 0.41 mmol) in MeOH (8 mL) and water (2 mL) was added lithium hydroxide (99 mg, 4.13 mmol) and the whole mixture was stirred for 12 hours at room temperature. After 12 hours, pH was adjusted to 1-2 with a 1 M HCl solution, the whole mixture was then transferred to a separating funnel, and extracted twice with DCM. The combined organic layers were then dried over sodium sulfate, filtered and concentrated in vacuo. The resulting residue was triturated in diethyl ether with few drops of DCM, then filtered to give 108 mg (47% yield) of (trans)-4-[7-[1-[4-(trifluoromethoxy)benzoyl]-4-piperidyl]-3H-imidazo[4,5-b]pyridin-2-yl]cyclohexanecarboxylic acid;hydrochloride as a pale brown solid. LC/MS (m/z, M+H-HCl, Method 1): calc. 517.2, found 517.5.

### Examples 277 and 278: (cis)-[4-[2-[3-aminotetrahydrofuran-2-yl]-3H-imidazo[4,5-b]pyridin-7-yl]-1-piperidyl]-[2-amino-4-(trifluoromethoxy)phenyl]methanone, Isomers 1 and 2

Examples 277 and 278 were prepared following a procedure similar to that of step 1 of Example 5 using rac-(2S,3R)-3-(tert-butoxycarbonylamino)tetrahydrofuran-2-carboxylic acid and tert-butyl N-[2-[4-(2,3-diamino-4-pyridyl)piperidine-1-carbonyl]-5-(trifluoromethoxy)phenyl]carbamate (described in step 2 of Examples 121 and 122), followed by a procedure similar to step 3 of Example 29, followed by chiral separation using Method CS3.

### Example 279: (rac)-[4-[2-(1,1-dioxothiolan-3-yl)-3H-imidazo[4,5-b]pyridin-7-yl]-1-piperidyl]-[4-(trifluoromethoxy)phenyl]methanone

Example 279 was prepared following a procedure similar to that of step 1 of Example 15 using [4-(2,3-diamino-4-pyridyl)-1-piperidyl]-[4-(trifluoromethoxy)phenyl]methanone and the corresponding carboxylic acid.

### Example 280: [4-[2-(4-hydroxy-4-methyl-cyclohexyl)-3H-imidazo[4,5-b]pyridin-7-yl]-1-piperidyl]-[4-(trifluoromethoxy)phenyl]methanone

Example 280 was prepared following a procedure similar to that of step 1 of Example 15 using [4-(2,3-diamino-4-pyridyl)-1-piperidyl]-[4-(trifluoromethoxy)phenyl]methanone and the corresponding carboxylic acid.

### Example 281: [2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;hydrochloride

Example 281 was prepared following a procedure similar to that of step 1 of Example 5 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to step 4 of Examples 1 and 2.

### Example 282: [2-amino-4-(trifluoromethoxy)phenyl]-[4-[2-(4-hydroxy-4-methyl-cyclohexyl)-3H-imidazo[4,5-b]pyridin-7-yl]-1-piperidyl]methanone;hydrochloride

Example 282 was prepared following a procedure similar to that of step 1 of Example 5 using tert-butyl N-[2-[4-(2,3-diamino-4-pyridyl)piperidine-1-carbonyl]-5-(trifluoromethoxy)phenyl]carbamate and the corresponding carboxylic acid, followed by a procedure similar to step 4 of Examples 1 and 2. LC/MS (m/z, M+H): 518

### Examples 283 and 284: [2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;hydrochloride, Isomers 1 and 2

Examples 283 and 284 were obtained by chiral separation of the compound of Example 281 using Method CS4.

Example 283 (Isomer 1): LC/MS (m/z, M+H): 572

Example 284 (Isomer 2): LC/MS (m/z, M+H): 572

### Example 285: [2-amino-4-(trifluoromethoxy)phenyl]-[4-[2-(1,2,3,6-tetrahydropyridin-4-yl)-3H-imidazo[4,5-b]pyridin-7-yl]-1-piperidyl]methanone

Example 285 was prepared following a procedure similar to that of Examples 261 and 262 using 1-(tert-butoxycarbonyl)-3-fluoropiperidine-4-carboxylic acid and [2-amino-4-(trifluoromethoxy)phenyl]-[4-(2,3-diamino-4-pyridyl)-1-piperidyl]methanone (prepared following the procedure of step 8 of Examples 355 and 356 using 4-(4-piperidyl)pyridine-2,3-diamine;hydrochloride (step 1 of Example 74) and 2-amino-4-(trifluoromethoxy)benzoic acid) in step 1.

### Examples 286 and 287: [4-[2-[azepan-3-yl]-3H-imidazo[4,5-b]pyridin-7-yl]-1-piperidyl]-[4-(trifluoromethoxy)phenyl]methanone, Isomers 1 and 2

Examples 286 and 287 were prepared according to Example 252, followed by chiral separation using Method CS11.

Example 286 (Isomer 1): LC/MS (m/z, M+H): 488

Example 287 (Isomer 2): LC/MS (m/z, M+H): 488

### Examples 288 and 289: [2-amino-4-(trifluoromethoxy)phenyl]-[4-[2-[azepan-3-yl]-3H-imidazo[4,5-b]pyridin-7-yl]-1-piperidyl]methanone, Isomers 1 and 2

Examples 288 and 289 were prepared following a procedure similar to that of Example 252, followed by chiral separation using Method CS3.

Example 288 (Isomer 1): LC/MS (m/z, M+H): 503

Example 289 (Isomer 2): LC/MS (m/z, M+H): 503

### Example 290: [2-amino-4-(pentafluoro-λ⁶-sulfanyl)phenyl]-[4-(2-cyclopentyl-3H-imidazo[4,5-b]pyridin-7-yl)-1-piperidyl]methanone

Example 290 was prepared following a procedure similar to that of step 1 of Example 5 using cyclopentanecarboxylic acid and tert-butyl 4-(2,3-diamino-4-pyridyl)piperidine-1-carboxylate (described in step 2 of Examples 1 and 2), followed by a procedure similar to that of step 3 of Example 29, followed by a procedure similar to that of step 8 of Examples 355 and 356 using 2-amino-4-(pentafluoro-λ⁶-sulfanyl)benzoic acid. LC/MS (m/z, M+H): 516; ¹H NMR (400 MHz, DMSO-d6, 100° C.) δ ppm 1.57-2.19 (m, 12H), 3.06-3.18 (m, 2H), 3.30 (quin, J=8.1 Hz, 1H), 3.33-3.52 (m, 1H), 4.06-4.24 (m, 2H), 5.46 (s, 2H), 6.95-7.05 (m, 2H), 7.23 (br d, J=8.3 Hz, 1H), 7.27 (d, J=2.3 Hz, 1H), 8.12 (br d, J=4.9 Hz, 1H), 12.03-12.54 (m, 1H)

### Example 291: [4-(2-cyclopentyl-3H-imidazo[4,5-b]pyridin-7-yl)-1-piperidyl]-[4-(pentafluoro-λ⁶-sulfanyl)phenyl]methanone

Example 291 was prepared following a procedure similar to that of step 1 of Example 5 using cyclopentanecarboxylic acid and tert-butyl 4-(2,3-diamino-4-pyridyl)piperidine-1-carboxylate (described in step 2 of Examples 1 and 2), followed by a procedure similar to that of step 3 of Example 29, followed by a procedure similar to that of step 8 of Examples 355 and 356 using 4-(pentafluorothio)benzoic acid. LC/MS (m/z, M+H): 501; ¹H NMR (400 MHz, DMSO-d6, 100° C.) δ ppm 1.58-2.13 (m, 12H), 3.13 (br t, J=10.8 Hz, 2H), 3.30 (quin, J=8.1 Hz, 1H), 3.37-3.55 (m, 1H), 3.89-4.61 (m, 2H), 7.00 (d, J=5.0 Hz, 1H), 7.65 (br d, J=8.9 Hz, 2H), 7.94 (d, J=8.9 Hz, 2H), 8.08-8.18 (m, 1H), 11.80-12.63 (m, 1H)

### Example 292: (rac)-[2-amino-4-(pentafluoro-λ⁶-sulfanyl)phenyl]-[4-(2-tetrahydrofuran-2-yl-3H-imidazo[4,5-b]pyridin-7-yl)-1-piperidyl]methanone

Example 292 was prepared following a procedure similar to that of step 1 of Example 5 using 2-tetrahydrofuroic acid and tert-butyl 4-(2,3-diamino-4-pyridyl)piperidine-1-carboxylate (described in Step 2 of Examples 1 and 2), followed by a procedure similar to that of step 3 of Example 29, followed by a procedure similar to that of step 8 of Examples 355 and 356 using 2-amino-4-(pentafluoro-λ⁶-sulfanyl)benzoic acid. LC/MS (m/z, M+H): 518; ¹H NMR (400 MHz, DMSO-d6, 100° C.) δ ppm 1.80-2.17 (m, 6H), 2.22-2.43 (m, 2H), 3.09-3.20 (m, 2H), 3.42-3.58 (m, 1H), 3.81-3.94 (m, 1H), 3.98-4.07 (m, 1H), 4.09-4.25 (m, 2H), 5.10 (t, J=6.9 Hz, 1H), 5.49 (s, 2H), 7.03 (dd, J=8.5, 2.3 Hz, 1H), 7.06 (d, J=5.0 Hz, 1H), 7.26 (br d, J=8.5 Hz, 1H), 7.30 (d, J=2.3 Hz, 1H), 8.22 (d, J=5.0 Hz, 1H), 12.34-12.69 (m, 1H)

### Example 293 (cis)-[4-[2-(4-Methoxycyclohexyl)-3H-imidazo[4,5-b]pyridin-7-yl]-1-piperidyl]-[4-(pentafluoro-λ⁶-sulfanyl)phenyl]methanone

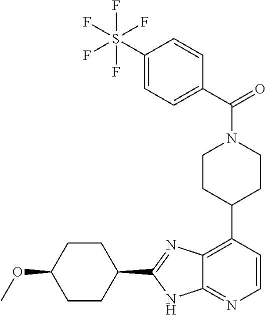 [SMILES: CO[C@H]1CC[C@@H](c2nc3c(C4CCN(C(=O)c5ccc(S(F)(F)(F)(F)F)cc5)CC4)ccnc3[nH]2)CC1]

### Step 1: (cis) & (trans)-tert-Butyl 4-[2-(4-methoxycyclohexyl)-3H-imidazo[4,5-b]pyridin-7-yl]piperidine-1-carboxylate

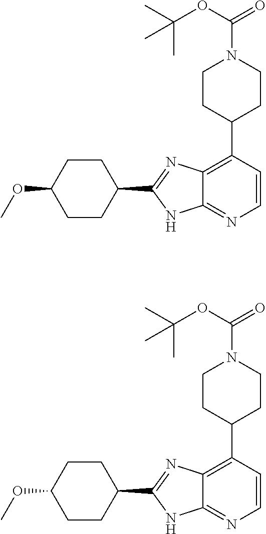 [SMILES: CO[C@H]1CC[C@@H](c2nc3c(C4CCN(C(=O)OC(C)(C)C)CC4)ccnc3[nH]2)CC1 ; CO[C@H]1CC[C@H](c2nc3c(C4CCN(C(=O)OC(C)(C)C)CC4)ccnc3[nH]2)CC1]

In a 10-20 mL microwave vial, were placed 4-methoxycyclohexanecarboxylic acid (300 mg, 1.9 mmol), CDI (400 mg, 2.46 mmol) in 10 mL of acetonitrile. The vial was sealed and submitted to microwave at 135° C. for 15 minutes. Then, the vial was opened, and tert-butyl 4-(2,3-diamino-4-pyridyl)piperidine-1-carboxylate (527 mg, 1.80 mmol) was added. The vial was sealed and submitted to microwave for 60 minutes at 135° C. After 60 minutes, the vial was opened and the reaction mixture was concentrated to dryness. The resulting residue was purified by flash chromatography on silica gel (SiO2 80 g) eluting with DCM/MeOH/NH₄OH 100/0/0 to 70/30/3, to give 250 mg (32% yield) of (cis)-tert-butyl 4-[2-(4-methoxycyclohexyl)-3H-imidazo[4,5-b]pyridin-7-yl]piperidine-1-carboxylate and 180 mg (23% yield) of (trans)-tert-butyl 4-[2-(4-methoxycyclohexyl)-3H-imidazo[4,5-b]pyridin-7-yl]piperidine-1-carboxylate as pale brown solids. LC/MS (m/z, M+H, Method 1): (cis) calc. 415.5, found 415.2; (trans) calc. 415.5, found 415.2.

### Step 2: (cis)-2-(4-Methoxycyclohexyl)-7-(4-piperidyl)-3H-imidazo[4,5-b]pyridine; hydrochloride

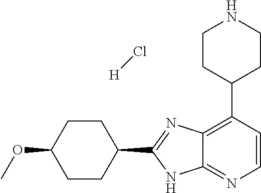 [SMILES: CO[C@H]1CC[C@@H](c2nc3c(C4CCNCC4)ccnc3[nH]2)CC1 ; Cl]

To a solution of (cis)-tert-butyl 4-[2-(4-methoxycyclohexyl)-3H-imidazo[4,5-b]pyridin-7-yl]piperidine-1-carboxylate (250 mg, 0.6 mmol) in methanol (2 mL) and ethyl acetate (6 mL), was added 1.96 mL of a 4 M HCl solution. The resulting reaction mixture was then stirred at room temperature for 12 hours. After 12 hours, the reaction mixture was concentrated to dryness, triturated in diethyl ether, filtered, and washed with diethyl ether to give 212 mg (100% yield) of (cis)-2-(4-methoxycyclohexyl)-7-(4-piperidyl)-3H-imidazo[4,5-b]pyridine;hydrochloride as a white solid. LC/MS (m/z, M+H-HCl, Method 1): calc. 315.2, found 315.2.

### Step 3: (cis)-[4-[2-(4-Methoxycyclohexyl)-3H-imidazo[4,5-b]pyridin-7-yl]-1-piperidyl]-[4-(pentafluoro-λ⁶-sulfanyl)phenyl]methanone

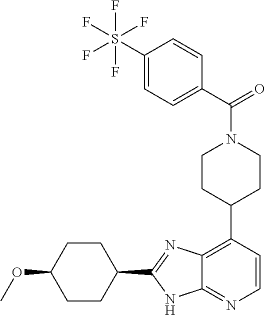 [SMILES: CO[C@H]1CC[C@@H](c2nc3c(C4CCN(C(=O)c5ccc(S(F)(F)(F)(F)F)cc5)CC4)ccnc3[nH]2)CC1]

To a solution of (cis)-2-(4-methoxycyclohexyl)-7-(4-piperidyl)-3H-imidazo[4,5-b]pyridine;hydrochloride (110 mg, 0.31 mmol) in DMF (0.8 mL) was added N,N-diisopropylethylamine (0.22 mL, 1.25 mmol) and the resulting reaction mixture was stirred at room temperature for 5 minutes. Then, 4-(pentafluoro-λ⁶-sulfanyl)benzoic acid (73 mg, 0.30 mmol) and TATU (122 mg, 0.36 mmol) were added. The resulting mixture was stirred at room temperature for two hours. After 2 hours, the reaction mixture was diluted with AcOEt, transferred in a separating funnel, extracted twice with EtOAc, washed with water. The combined organic layers were dried over magnesium sulfate, filtered and concentrated to dryness. The resulting residue was purified by flash chromatography on silica gel (SiO₂ 80 g) eluting with DCM/MeOH/NH₄OH 100/0/0 to 90/10/1, to give 72 mg (40% yield) of (cis)-[4-[2-(4-methoxycyclohexyl)-3H-imidazo[4,5-b]pyridin-7-yl]-1-piperidyl]-[4-(pentafluoro-λ⁶-sulfanyl)phenyl]methanone as a white solid. LC/MS (m/z, M+H-HCl, Method 1): calc. 545.2, found 545.2; ¹H NMR (400 MHz, DMSO-d6, 30° C.) δ ppm 1.49-1.66 (m, 2H) 1.72-2.08 (m, 10H) 2.90-3.05 (m, 2H) 3.26 (s, 3H) 3.27-3.36 (m, 1H) 3.42-3.53 (m, 2H) 3.55-3.72 (m, 2H) 4.60-4.79 (m, 1H) 7.09 (d, J=5.1 Hz, 1H) 7.69 (d, J=8.5 Hz, 2H) 7.98 (d, J=8.5 Hz, 2H) 8.20 (d, J=5.1 Hz, 1H)

### Example 294: (trans)-[4-[2-(4-methoxycyclohexyl)-3H-imidazo[4,5-b]pyridin-7-yl]-1-piperidyl]-[4-(pentafluoro-λ⁶-sulfanyl)phenyl]methanone

Example 294 was prepared following a procedure similar to that of steps 2 and 3 of Example 293 using (trans)-tert-butyl 4-[2-(4-methoxycyclohexyl)-3H-imidazo[4,5-b]pyridin-7-yl]piperidine-1-carboxylate. LC/MS (m/z, M+H): 545; ¹H NMR (400 MHz, DMSO-d6, 100° C.) δ ppm 1.20-1.37 (m, 2H) 1.62-1.78 (m, 4H) 1.81-2.02 (m, 2H) 2.05-2.18 (m, 4H) 2.73-3.26 (m partially hidden, 4H) 3.28 (s, 3H) 3.35-3.51 (m, 1H) 3.94-4.30 (m, 2H) 7.01 (d, J=5.0 Hz, 1H) 7.66 (d, J=8.5 Hz, 2H) 7.94 (d, J=8.6 Hz, 2H) 8.15 (d, J=5.0 Hz, 1H)

### Example 295: (rac)-[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone

Example 295 was prepared following a procedure similar to that of Examples 332 and 333 using tert-butyl 4-(4,4,5,5-tetramethyl-1,3,2-dioxaborolan-2-yl)-2,3,6,7-tetrahydro-1H-azepine-1-carboxylate in step 1, and 2-amino-4-(pentafluoro-λ⁶-sulfanyl)benzoic acid in step 4. LC/MS (m/z, M+H): 546; ¹H NMR (400 MHz, DMSO-d6 with acetic acid-d3, 100° C.) δ ppm 1.73-2.18 (m, 10H), 3.10-3.21 (m, 1H), 3.31-3.58 (m, 5H), 3.61-3.92 (m, 2H), 3.96 (dt, J=11.4, 3.4 Hz, 2H), 6.96-7.06 (m, 2H), 7.23-7.31 (m, 2H), 8.14 (d, J=5.0 Hz, 1H)

### Example 296: (cis)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone

Example 296 was prepared following a procedure similar to that of step 3 of Example 293 using 2-amino-4-(pentafluoro-λ⁶-sulfanyl)benzoic acid. LC/MS (m/z, M+H): 560; ¹H NMR (400 MHz, DMSO-d6, 100° C.) δ ppm 1.52-2.17 (m, 12H) 3.14 (br t, J=11.8 Hz, 3H) 3.29 (s, 3H) 3.37-3.54 (m, 2H) 4.07-4.26 (m, 2H) 5.48 (br s, 2H) 6.94-7.08 (m, 2H) 7.21-7.34 (m, 2H) 8.15 (br s, 1H) 11.81-12.66 (m, 1H)

### Example 297: (trans)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone

Example 297 was prepared following a procedure similar to that of Examples 355 and 356. LC/MS (m/z, M+H): 560; ¹H NMR (400 MHz, DMSO-d6, 100° C.) δ ppm 1.21-1.39 (m, 2H) 1.60-1.78 (m, 2H) 1.92 (br s, 4H) 2.11 (br d, J=10.5 Hz, 4H) 2.75-2.89 (m, 1H) 3.05-3.15 (m, 2H) 3.16-3.26 (m, 1H) 3.28 (s, 3H) 3.33-3.50 (m, 1H) 4.02-4.21 (m, 2H) 5.45 (br s, 2H) 6.94-7.03 (m, 2H) 7.24 (d, J=8.4 Hz, 1H) 7.27 (d, J=2.2 Hz, 1H) 8.13 (br d, J=4.4 Hz, 1H) 11.50-12.62 (m, 1H)

### Examples 298 and 299: [4-(pentafluoro-λ⁶-sulfanyl)phenyl]-[4-[2-[tetrahydrofuran-2-yl]-3H-imidazo[4,5-b]pyridin-7-yl]-1-piperidyl]methanone, Isomers 1 and 2

Examples 298 and 299 were obtained by chiral separation of the compound of Example 34 using Method CS1.

Example 298: LC/MS (m/z, M+H): 503; ¹H NMR (400 MHz, DMSO-d6, 30° C.) δ ppm 1.69-2.08 (m, 6H) 2.13-2.24 (m, 1H) 2.27-2.38 (m, 1H) 2.90-3.05 (m, 1H) 3.21-3.40 (m, 1H) 3.45-3.54 (m, 1H) 3.54-3.63 (m, 1H) 3.80-3.92 (m, 1H) 3.96-4.05 (m, 1H) 4.62-4.73 (m, 1H) 5.02-5.13 (m, 1H) 7.11 (d, J=5.0 Hz, 1H) 7.69 (br d, J=8.3 Hz, 2H) 8.00 (d, J=8.3 Hz, 2H) 8.17-8.26 (m, 1H) 12.57-13.08 (m, 1H)

Example 299: LC/MS (m/z, M+H): 503; ¹H NMR (400 MHz, DMSO-d6, 30° C.) δ ppm 1.69-2.08 (m, 6H) 2.13-2.24 (m, 1H) 2.27-2.38 (m, 1H) 2.90-3.05 (m, 1H) 3.21-3.40 (m, 1H) 3.45-3.54 (m, 1H) 3.54-3.63 (m, 1H) 3.80-3.92 (m, 1H) 3.96-4.05 (m, 1H) 4.62-4.73 (m, 1H) 5.02-5.13 (m, 1H) 7.11 (d, J=5.0 Hz, 1H) 7.69 (br d, J=8.3 Hz, 2H) 8.00 (d, J=8.3 Hz, 2H) 8.17-8.26 (m, 1H) 12.57-13.08 (m, 1H)

### Examples 300 and 301: [4-(pentafluoro-λ⁶-sulfanyl)phenyl]-[4-[2-[tetrahydrofuran-3-yl]-3H-imidazo[4,5-b]pyridin-7-yl]-1-piperidyl]methanone, Isomers 1 and 2

Examples 300 and 301 were prepared following a procedure similar to that of Example 293 using tetrahydrofuran-3-carboxylic acid in step 1 and 4-(pentafluoro-λ⁶-sulfanyl)benzoic acid in step 3, followed by chiral separation using Method CS12.

Example 300: LC/MS (m/z, M+H): 503; ¹H NMR (400 MHz, DMSO-d6, 100° C.) δ ppm 1.80-2.04 (m, 4H) 2.34 (q, J=7.3 Hz, 2H) 3.13 (br t, J=11.2 Hz, 2H) 3.36-3.52 (m, 1H) 3.67 (quin, J=7.5 Hz, 1H) 3.77-4.34 (m, 6H) 7.03 (d, J=5.0 Hz, 1H) 7.66 (d, J=8.5 Hz, 2H) 7.94 (d, J=8.5 Hz, 2H) 8.16 (d, J=5.0 Hz, 1H) 11.87-12.80 (m, 1H)

Example 301: LC/MS (m/z, M+H): 503; ¹H NMR (400 MHz, DMSO-d6, 100° C.) δ ppm 1.85-2.03 (m, 4H) 2.28-2.41 (m, 2H) 3.03-3.28 (m, 2H) 3.37-3.52 (m, 1H) 3.68 (quin, J=7.5 Hz, 1H) 3.78-4.32 (m, 6H) 7.04 (d, J=5.0 Hz, 1H) 7.66 (d, J=8.5 Hz, 2H) 7.94 (d, J=8.5 Hz, 2H) 8.17 (d, J=5.0 Hz, 1H)

### Example 302: [4-[2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone

Example 302 was prepared following a procedure similar to that of Example 293 using 3-methoxybicyclo[1.1.1]pentane-1-carboxylic acid in step 1, without MeOH as co-solvent in step 2, and using 4-(pentafluoro-λ⁶-sulfanyl)benzoic acid in step 3. LC/MS (m/z, M+H): 529; ¹H NMR (400 MHz, DMSO-d6, 100° C.) δ ppm 1.76-2.02 (m, 4H) 2.31 (s, 6H) 3.14 (br t, J=12.4 Hz, 2H) 3.29 (s, 3H) 3.42-3.57 (m, 1H) 3.92-4.35 (m, 2H) 7.04 (d, J=5.0 Hz, 1H) 7.66 (d, J=8.4 Hz, 2H) 7.94 (d, J=8.4 Hz, 2H) 8.17 (d, J=5.0 Hz, 1H) 11.98-13.10 (m, 1H)

### Examples 303: [4-(pentafluoro-λ⁶-sulfanyl)phenyl]-[4-[2-(4-piperidyl)-3H-imidazo[4,5-b]pyridin-7-yl]-1-piperidyl]methanone;hydrochloride

Example 303 was prepared following a procedure similar to that of step 1 of Example 5 using [4-(2,3-diamino-4-pyridyl)-1-piperidyl]-[4-(pentafluoro-λ⁶-sulfanyl)phenyl]methanone (described in step 1 of Example 408) and 1-(tert-butoxycarbonyl)piperidine-4-carboxylic acid, followed by a procedure similar to that of step 4 of Examples 1 and 2 (using DCM/MeOH 9/1 as solvent). LC/MS (m/z, M+H): 516; ¹H NMR (400 MHz, DMSO-d6 with acetic acid-d3, 100° C.) δ ppm 1.91-2.02 (m, 4H), 2.14 (dtd, J=14.3, 10.7, 10.7, 3.9 Hz, 2H), 2.24-2.36 (m, 2H), 3.08-3.24 (m, 4H), 3.26-3.38 (m, 1H), 3.38-3.55 (m, 3H), 4.01-4.42 (m, 2H), 7.15 (d, J=5.1 Hz, 1H), 7.67 (br d, J=8.8 Hz, 2H), 7.95 (d, J=8.8 Hz, 2H), 8.26 (d, J=5.1 Hz, 1H)

### Example 304: [4-[2-[(2R)-morpholin-2-yl]-3H-imidazo[4,5-b]pyridin-7-yl]-1-piperidyl]-[4-(pentafluoro-λ⁶-sulfanyl)phenyl]methanone

Example 304 was prepared following a procedure similar to that of step 1 of Example 5 using (R)-4-(tert-butoxycarbonyl)morpholine-2-carboxylic acid and [4-(2,3-diamino-4-pyridyl)-1-piperidyl]-[4-(pentafluoro-λ⁶-sulfanyl)phenyl]methanone (described in step 1 of Example 408), followed by a procedure similar to that of step 4 of Examples 1 and 2 (using DCM as solvent instead of MeOH). LC/MS (m/z, M+H): 518; ¹H NMR (400 MHz, DMSO-d6, 100° C.) δ ppm 1.75-2.03 (m, 4H) 2.75-3.22 (m, 6H) 3.43-3.56 (m, 1H) 3.61-3.74 (m, 1H) 3.88 (dt, J=11.1, 2.8 Hz, 1H) 3.95-4.30 (m, 2H) 4.69 (dd, J=9.5, 2.9 Hz, 1H) 7.06 (d, J=5.0 Hz, 1H) 7.66 (d, J=8.4 Hz, 2H) 7.94 (d, J=8.4 Hz, 2H) 8.21 (d, J=5.0 Hz, 1H)

### Example 305: [2-amino-4-(pentafluoro-λ⁶-sulfanyl)phenyl]-[4-[2-(3-methoxy-1-bicyclo[1.1.1]pentanyl)-3H-imidazo[4,5-b]pyridin-7-yl]-1-piperidyl]methanone

Example 305 was prepared following a procedure similar to that of Example 293 using 3-methoxybicyclo[1.1.1]pentane-1-carboxylic acid in step 1, without MeOH as co-solvent in step 2, and using 2-amino-4-(pentafluoro-λ⁶-sulfanyl)benzoic acid in step 3. LC/MS (m/z, M+H): 544; ¹H NMR (400 MHz, DMSO-d6 with acetic acid-d3, 100° C.) δ ppm 1.75-2.01 (m, 4H) 2.30 (s, 6H) 3.04-3.20 (m, 2H) 3.29 (s, 3H) 3.39-3.53 (m, 1H) 4.13 (br d, J=9.0 Hz, 2H) 5.45 (br s, 2H) 6.95-7.08 (m, 2H) 7.18-7.29 (m, 2H) 8.16 (br d, J=4.9 Hz, 1H) 11.98-12.97 (m, 1H)

### Example 306: [4-(2-N-morpholino-3H-imidazo[4,5-b]pyridin-7-yl)-1-piperidyl]-[4-(pentafluoro-λ⁶-sulfanyl)phenyl]methanone

Example 306 was prepared following a procedure similar to that of step 8 of Examples 355 and 356 using 4-(pentafluoro-λ⁶-sulfanyl)benzoic acid and 4-[7-(4-piperidyl)-3H-imidazo[4,5-b]pyridin-2-yl]morpholine;hydrobromide (described in step 1 of Example 249). LC/MS (m/z, M+H): 518; ¹H NMR (500 MHz, DMSO-d6, 25° C.) δ ppm 1.65-2.01 (m, 4H) 2.86-3.00 (m, 1H) 3.16-3.39 (m partially hidden, 2H) 3.44-3.62 (m, 5H) 3.66-3.78 (m, 4H) 4.56-4.74 (m, 1H) 6.91 (br s, 1H) 7.68 (br d, J=7.6 Hz, 2H) 7.86 (br s, 1H) 8.00 (d, J=8.7 Hz, 2H) 10.97-12.23 (m, 1H)

### Example 307: [2-amino-4-(pentafluoro-λ⁶-sulfanyl)phenyl]-[4-[2-(4-piperidyl)-3H-imidazo[4,5-b]pyridin-7-yl]-1-piperidyl]methanone;hydrochloride

Example 307 was prepared following a procedure similar to that of step 1 of Example 5 using [2-amino-4-(pentafluoro-λ⁶-sulfanyl)phenyl]-[4-(2,3-diamino-4-pyridyl)-1-piperidyl]methanone (prepared following a procedure similar to step 1 of Example 408 using 2-amino-4-(pentafluoro-λ⁶-sulfanyl)benzoic acid) and 1-(tert-butoxycarbonyl)piperidine-4-carboxylic acid, followed by a procedure similar to that of step 4 of Examples 1 and 2 (using DCM/MeOH 9/1 as solvent). LC/MS (m/z, M+H): 531; ¹H NMR (400 MHz, DMSO-d6, 100° C.) δ ppm 1.81-2.02 (m, 4H), 2.11-2.26 (m, 2H), 2.27-2.41 (m, 2H), 3.03-3.19 (m, 4H), 3.33-3.47 (m, 3H), 3.61-3.72 (m, 1H), 4.03-4.24 (m, 2H), 7.00 (dd, J=8.4, 2.1 Hz, 1H), 7.25 (br d, J=8.4 Hz, 1H), 7.28 (d, J=2.1 Hz, 1H), 7.32 (br d, J=5.4 Hz, 1H), 8.35 (d, J=5.4 Hz, 1H), 8.80-9.27 (m, 2H)

### Examples 308 and 312: [2-amino-4-(pentafluoro-λ⁶-sulfanyl)phenyl]-[4-[2-[tetrahydrofuran-3-yl]-3H-imidazo[4,5-b]pyridin-7-yl]-1-piperidyl]methanone, Isomers 1 and 2

Examples 308 and 312 were prepared following a procedure similar to that of Example 293 using tetrahydrofuran-3-carboxylic acid in step 1 and 2-amino-4-(pentafluoro-λ⁶-sulfanyl)benzoic acid in step 3, followed by chiral separation using Method CS12.

Example 308 (Isomer 1): LC/MS (m/z, M+H): 518; ¹H NMR (400 MHz, DMSO-d6, 100° C.) δ ppm 1.78-2.04 (m, 4H) 2.34 (q, J=7.4 Hz, 2H) 3.02-3.20 (m, 2H) 3.31-3.53 (m, 1H) 3.67 (quin, J=7.4 Hz, 1H) 3.83 (q, J=7.4 Hz, 1H) 3.88-3.99 (m, 2H) 4.02-4.22 (m, 3H) 5.46 (br s, 2H) 6.95-7.06 (m, 2H) 7.23 (d, J=8.5 Hz, 1H) 7.27 (d, J=2.3 Hz, 1H) 8.10-8.23 (m, 1H) 12.07-12.84 (m, 1H)

Example 312 (Isomer 2): LC/MS (m/z, M+H): 518; ¹H NMR (400 MHz, DMSO-d6, 100° C.) δ ppm 1.78-2.04 (m, 4H) 2.34 (q, J=7.4 Hz, 2H) 3.02-3.20 (m, 2H) 3.31-3.53 (m, 1H) 3.67 (quin, J=7.4 Hz, 1H) 3.83 (q, J=7.4 Hz, 1H) 3.88-3.99 (m, 2H) 4.02-4.22 (m, 3H) 5.46 (br s, 2H) 6.95-7.06 (m, 2H) 7.23 (d, J=8.5 Hz, 1H) 7.27 (d, J=2.3 Hz, 1H) 8.10-8.23 (m, 1H) 12.07-12.84 (m, 1H)

### Examples 309 and 310: (cis)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone and (trans)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone

Examples 309 and 310 were prepared following a procedure similar to that of Example 293 using 3-methoxycyclobutanecarboxylic acid in step 1, without MeOH as co-solvent in step 2, and 2-amino-4-(pentafluoro-λ⁶-sulfanyl)benzoic acid in step 3, followed by isomeric separation using Method CS12.

Example 309 (cis-isomer): LC/MS (m/z, M+H): 532; ¹H NMR (400 MHz, DMSO-d6, 100° C.) δ ppm 1.72-2.06 (m, 4H) 2.33-2.44 (m, 2H) 2.59-2.71 (m, 2H) 3.04-3.18 (m, 2H) 3.20 (s, 3H) 3.34-3.55 (m, 1H) 3.60-3.74 (m, 1H) 4.16 (br d, J=5.0 Hz, 3H) 5.46 (br s, 2H) 6.94-7.05 (m, 2H) 7.24 (br d, J=8.5 Hz, 1H) 7.27 (d, J=2.3 Hz, 1H) 8.06-8.23 (m, 1H) 11.87-12.79 (m, 1H)

Example 310 (trans-isomer): LC/MS (m/z, M+H): 532; ¹H NMR (400 MHz, DMSO-d6 with acetic acid-d3, 100° C.) δ ppm 1.89-2.01 (m, 4H) 2.24-2.38 (m, 2H) 2.60-2.74 (m, 2H) 3.05-3.17 (m, 2H) 3.18-3.30 (m, 1H) 3.21 (s, 3H) 3.36-3.52 (m, 1H) 3.85-3.98 (m, 1H) 4.05-4.20 (m, 2H) 6.95-7.06 (m, 2H) 7.25 (d, J=8.4 Hz, 1H) 7.27 (d, J=2.1 Hz, 1H) 8.15 (d, J=5.0 Hz, 1H)

### Example 311: [2-amino-4-(pentafluoro-λ⁶-sulfanyl)phenyl]-[4-(2-N-morpholino-3H-imidazo[4,5-b]pyridin-7-yl)-1-piperidyl]methanone

Example 311 was prepared by a procedure similar to that of step 2 of Example 249 using 2-bromo-7-(4-piperidyl)-3H-imidazo[4,5-b]pyridine;hydrobromide and morpholine (without DMF), followed by a procedure similar to that of step 8 of Examples 355 and 356 using 2-amino-4-(pentafluoro-λ⁶-sulfanyl)benzoic acid. LC/MS (m/z, M+H): 533; ¹H NMR (400 MHz, DMSO-d6, 100° C.) δ ppm 1.88 (br s, 4H) 3.02-3.15 (m, 2H) 3.21-3.34 (m, 1H) 3.50-3.59 (m, 4H) 3.68-3.77 (m, 4H) 4.01-4.18 (m, 2H) 5.44 (br s, 2H) 6.82 (br d, J=4.8 Hz, 1H) 7.00 (dd, J=8.4, 2.1 Hz, 1H) 7.22 (br d, J=8.4 Hz, 1H) 7.27 (d, J=2.1 Hz, 1H) 7.77-7.91 (m, 1H) 11.14-12.09 (m, 1H)

### Example 313: (trans)-[4-[2-(3-methoxycyclobutyl)-3H-imidazo[4,5-b]pyridin-7-yl]-1-piperidyl]-[4-(pentafluoro-λ⁶-sulfanyl)phenyl]methanone

Example 313 was prepared following a procedure similar to that of step 1 of Example 5 using tert-butyl 4-(2,3-diamino-4-pyridyl)piperidine-1-carboxylate (described in step 2 of Examples 1 and 2) and 3-methoxycyclobutanecarboxylic acid, followed by a procedure similar to that of step 3 of Example 29, followed by a procedure similar to that of step 1 of Examples 1 and 2 using 4-(pentafluoro-λ⁶-sulfanyl)benzoic acid, followed by isomeric separation using Method CS12. LC/MS (m/z, M+H): 517; ¹H NMR (400 MHz, DMSO-d6 with acetic acid-d3, 100° C.) δ ppm 1.89-2.02 (m, 4H), 2.35-2.45 (m, 2H), 2.62-2.73 (m, 2H), 3.15 (br t, J=11.3 Hz, 2H), 3.21 (s, 3H), 3.40-3.52 (m, 1H), 3.64-3.77 (m, 1H), 3.93-4.46 (m, 3H), 7.04 (d, J=5.1 Hz, 1H), 7.66 (br d, J=8.8 Hz, 2H), 7.93 (d, J=8.8 Hz, 2H), 8.17 (d, J=5.1 Hz, 1H)

### Example 314: [4-[2-(1-methyl-4-piperidyl)-3H-imidazo[4,5-b]pyridin-7-yl]-1-piperidyl]-[4-(pentafluoro-λ⁶-sulfanyl)phenyl]methanone

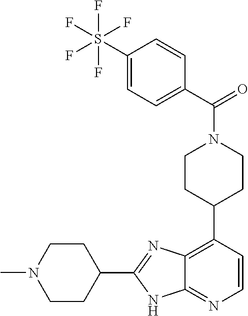 [SMILES: CN1CCC(c2nc3c(C4CCN(C(=O)c5ccc(S(F)(F)(F)(F)F)cc5)CC4)ccnc3[nH]2)CC1]

To a solution of [4-(pentafluoro-λ⁶-sulfanyl)phenyl]-[4-[2-(4-piperidyl)-3H-imidazo[4,5-b]pyridin-7-yl]-1-piperidyl]methanone (prepared according to Example 303 and beforehand eluted on a SCX column with 2M NH₃-MeOH to generate the free base; 51 mg, 0.1 mmol) in MeOH (4 mL), were added a 37% w/w solution of p-formaldehyde in water (40 mg, 0.49 mmol, 5 equivalents) and acetic acid (0.011 mL, 0.2 mmol). The resulting mixture was stirred at room temperature for 15 minutes and sodium cyanoborohydride (11 mg, 0.16 mmol) was added and the resulting reaction mixture was stirred at room temperature for 1.5 hours. The solution mixture was filtered and the filtrate was concentrated in vacuo. The resulting residue was purified on silica gel (SiO₂ 10 g) eluting with DCM/NH₃-MeOH 7M 4%, to give 38 mg (74% yield) of [4-[2-(1-methyl-4-piperidyl)-3H-imidazo[4,5-b]pyridin-7-yl]-1-piperidyl]-[4-(pentafluoro-λ⁶-sulfanyl)phenyl]methanone as a white solid. LC/MS (m/z, M+H, Method 1): calc. 530.2, found 530.2; ¹H NMR (400 MHz, DMSO-d6, 100° C.) δ ppm 1.80-2.10 (m, 8H), 2.21 (s, 3H), 2.73-3.05 (m partially hidden, 5H), 3.09-3.20 (m, 2H), 3.37-3.50 (m, 1H), 3.91-4.40 (m, 2H), 7.01 (d, J=5.1 Hz, 1H), 7.66 (br d, J=8.8 Hz, 2H), 7.94 (d, J=8.8 Hz, 2H), 8.14 (d, J=5.1 Hz, 1H), 11.60-12.91 (m, 1H)

### Example 315: [4-[2-(1-cyclopropyl-4-piperidyl)-3H-imidazo[4,5-b]pyridin-7-yl]-1-piperidyl]-[4-(pentafluoro-λ⁶-sulfanyl)phenyl]methanone

Example 315 was prepared by a procedure similar to that of Example 314 using (1-ethoxycyclopropoxy)trimethylsilane instead of p-formaldehyde. LC/MS (m/z, M+H): 556; ¹H NMR (400 MHz, DMSO-d6, 100° C.) δ ppm 0.27-0.35 (m, 2H), 0.38-0.47 (m, 2H), 1.62-1.72 (m, 1H), 1.77-2.01 (m, 8H), 2.33 (td, J=11.4, 2.8 Hz, 2H), 2.81-2.92 (m partially hidden, 1H), 3.03 (dt, J=11.6, 3.1 Hz, 2H), 3.08-3.21 (m, 2H), 3.36-3.51 (m, 1H), 3.96-4.41 (m, 2H), 7.00 (d, J=5.0 Hz, 1H), 7.65 (br d, J=8.8 Hz, 2H), 7.93 (d, J=8.8 Hz, 2H), 8.14 (d, J=5.0 Hz, 1H), 11.86-12.69 (m, 1H)

### Example 316: [4-[2-[(2S)-morpholin-2-yl]-3H-imidazo[4,5-b]pyridin-7-yl]-1-piperidyl]-[4-(pentafluoro-λ⁶-sulfanyl)phenyl]methanone

Example 316 was prepared by a procedure similar to that of step 1 of Example 5 using (S)-4-(tert-butoxycarbonyl)morpholine-2-carboxylic acid and 4-(pentafluoro-λ⁶-sulfanyl)phenyl]-[4-(2,3-diamino-4-pyridyl)-1-piperidyl]methanone (described in step 1 of Example 364), followed by a procedure similar to that of step 7 of Examples 355 and 356 (with DCM as solvent instead of MeOH). LC/MS (m/z, M+H): 518; ¹H NMR (400 MHz, DMSO-d6, 100° C.) δ ppm 1.77-2.02 (m, 4H) 2.77-3.23 (m partially hidden, 6H) 3.41-3.56 (m, 2H) 3.60-3.74 (m, 1H) 3.84-3.92 (m, 1H) 3.93-4.36 (m, 2H) 4.69 (dd, J=9.4, 2.7 Hz, 1H) 7.05 (d, J=5.1 Hz, 1H) 7.66 (br d, J=8.4 Hz, 2H) 7.94 (d, J=8.4 Hz, 2H) 8.21 (d, J=5.0 Hz, 1H)

### Example 317: (cis)-[4-[2-(3-methoxycyclobutyl)-3H-imidazo[4,5-b]pyridin-7-yl]-1-piperidyl]-[4-(pentafluoro-λ⁶-sulfanyl)phenyl]methanone

Example 317 was prepared following a procedure similar to that of step 1 of Example 5 using tert-butyl 4-(2,3-diamino-4-pyridyl)piperidine-1-carboxylate (described in step 2 of Examples 1 and 2) and 3-methoxycyclobutanecarboxylic acid, followed by a procedure similar to that of step 3 of Example 29, followed by a procedure similar to that of step 1 of Examples 1 and 2 using 4-(pentafluoro-λ⁶-sulfanyl)benzoic acid, followed by isomeric separation by Method CS12. LC/MS (m/z, M+H): 517; ¹H NMR (400 MHz, DMSO-d6 with acetic acid-d3, 100° C.) δ ppm 1.88-2.02 (m, 4H), 2.28-2.39 (m, 2H), 2.62-2.74 (m, 2H), 3.10-3.19 (m, 2H), 3.21 (s, 3H), 3.20-3.32 (m, 1H), 3.41-3.57 (m, 1H), 3.84-3.98 (m, 1H), 3.99-4.46 (m, 2H), 7.03 (d, J=5.0 Hz, 1H), 7.66 (br d, J=8.8 Hz, 2H), 7.93 (d, J=8.8 Hz, 2H), 8.17 (d, J=5.0 Hz, 1H)

### Example 318: [2-amino-4-(pentafluoro-λ⁶-sulfanyl)phenyl]-[4-[2-(1-methyl-4-piperidyl)-3H-imidazo[4,5-b]pyridin-7-yl]-1-piperidyl]methanone

Example 318 was prepared by a procedure similar to that of Example 314 using [2-amino-4-(pentafluoro-λ⁶-sulfanyl)phenyl]-[4-[2-(4-piperidyl)-3H-imidazo[4,5-b]pyridin-7-yl]-1-piperidyl]methanone (obtained from Example 307 eluted on SCX column with 2M NH₃-MeOH to generate the free base). LC/MS (m/z, M+H): 545; ¹H NMR (400 MHz, DMSO-d6, 100° C.) δ ppm 1.82-2.11 (m, 10H), 2.21 (s, 3H), 2.74-2.88 (m, 3H), 3.05-3.16 (m, 2H), 3.30-3.49 (m, 1H), 4.06-4.23 (m, 2H), 5.45 (s, 2H), 6.96-7.05 (m, 2H), 7.23 (d, J=8.4 Hz, 1H), 7.27 (d, J=2.1 Hz, 1H), 8.14 (br d, J=4.9 Hz, 1H), 12.01-12.68 (m, 1H)

### Example 319: [2-amino-4-(pentafluoro-λ⁶-sulfanyl)phenyl]-[4-[2-[(2S)-morpholin-2-yl]-3H-imidazo[4,5-b]pyridin-7-yl]-1-piperidyl]methanone

Example 319 was prepared by a procedure similar to that of step 1 of Example 5 using (S)-4-(tert-butoxycarbonyl)morpholine-2-carboxylic acid and [2-amino-4-(pentafluoro-λ⁶-sulfanyl)phenyl]-[4-(2,3-diamino-4-pyridyl)-1-piperidyl]methanone (prepared by a procedure similar to step 1 of Example 364), followed by a procedure similar to that of step 7 of Examples 355 and 356 (with DCM as solvent instead of MeOH). LC/MS (m/z, M+H): 533; ¹H NMR (400 MHz, DMSO-d6, 100° C.) δ ppm 1.74-2.05 (m, 4H) 2.75-3.24 (m, 6H) 3.38-3.55 (m, 1H) 3.61-3.73 (m, 1H) 3.82-3.94 (m, 1H) 4.13 (br d, J=9.3 Hz, 2H) 4.70 (dd, J=9.5, 2.8 Hz, 1H) 5.46 (br s, 2H) 7.00 (dd, J=8.4, 2.1 Hz, 1H) 7.04 (d, J=5.0 Hz, 1H) 7.24 (br d, J=8.4 Hz, 1H) 7.27 (d, J=2.1 Hz, 1H) 8.20 (d, J=5.0 Hz, 1H) 11.51-13.29 (m, 1H)

### Example 320: [2-amino-4-(pentafluoro-λ⁶-sulfanyl)phenyl]-[4-[2-(1-cyclopropyl-4-piperidyl)-3H-imidazo[4,5-b]pyridin-7-yl]-1-piperidyl]methanone

Example 320 was prepared by a procedure similar to that of Example 314 using (1-ethoxycyclopropoxy)trimethylsilane instead of p-formaldehyde and [2-amino-4-(pentafluoro-λ⁶-sulfanyl)phenyl]-[4-[2-(4-piperidyl)-3H-imidazo[4,5-b]pyridin-7-yl]-1-piperidyl]methanone (Example 307 eluted on SCX column with 2M NH₃-MeOH to generate the free base). LC/MS (m/z, M+H): 571; ¹H NMR (400 MHz, DMSO-d6, 100° C.) δ ppm 0.27-0.35 (m, 2H), 0.39-0.47 (m, 2H), 1.67 (tt, J=6.7, 3.5 Hz, 1H), 1.76-2.03 (m, 8H), 2.33 (td, J=11.5, 2.6 Hz, 2H), 2.81-2.92 (m, 1H), 2.99-3.06 (m, 2H), 3.06-3.16 (m, 2H), 3.34-3.48 (m, 1H), 4.06-4.22 (m, 2H), 5.45 (s, 2H), 6.96-7.03 (m, 2H), 7.23 (d, J=8.4 Hz, 1H), 7.27 (d, J=2.3 Hz, 1H), 8.04-8.44 (m, 1H), 12.00-12.57 (m, 1H)

### Example 321: [4-[2-(1-methoxy-1-methyl-ethyl)-3H-imidazo[4,5-b]pyridin-7-yl]-1-piperidyl]-[4-(pentafluoro-λ⁶-sulfanyl)phenyl]methanone

Example 321 was prepared following a procedure similar to that of Example 293 using 2-methoxy-2-methyl-propanoic acid in step 1, without MeOH as co-solvent in step 2, and using 4-(pentafluoro-λ⁶-sulfanyl)benzoic acid in step 3. LC/MS (m/z, M+H): 505; ¹H NMR (400 MHz, DMSO-d6, 100° C.) δ ppm 1.62 (s, 6H) 1.76-2.06 (m, 4H) 3.10 (s, 3H) 3.10-3.22 (m, 2H) 3.43-3.57 (m, 1H) 3.96-4.34 (m, 2H) 7.05 (d, J=5.0 Hz, 1H) 7.66 (d, J=8.5 Hz, 2H) 7.94 (d, J=8.5 Hz, 2H) 8.20 (br d, J=5.0 Hz, 1H) 12.03-12.76 (m, 1H)

### Example 322: [2-(methylamino)-4-(pentafluoro-λ⁶-sulfanyl)phenyl]-[4-[2-(1-methyl-4-piperidyl)-3H-imidazo[4,5-b]pyridin-7-yl]-1-piperidyl]methanone

Example 322 was prepared following a procedure similar to that of Example 314 using [2-amino-4-(pentafluoro-λ⁶-sulfanyl)phenyl]-[4-[2-(4-piperidyl)-3H-imidazo[4,5-b]pyridin-7-yl]-1-piperidyl]methanone (obtained from Example 307 eluted on SCX column with 2M NH₃-MeOH to generate the free base). LC/MS (m/z, M+H): 559; ¹H NMR (400 MHz, DMSO-d6, 100° C.) δ ppm 1.75-2.12 (m, 8H) 2.21 (s, 3H) 2.72-2.89 (m, 8H) 3.00-3.18 (m, 2H) 3.28-3.55 (m, 1H) 3.88-4.31 (m, 2H) 5.51-5.70 (m, 1H) 6.95 (d, J=2.1 Hz, 1 H) 6.99 (d, J=5.1 Hz, 1H) 7.05 (dd, J=8.4, 2.1 Hz, 1H) 7.26 (d, J=8.4 Hz, 1H) 8.06-8.23 (m, 1H) 12.00-12.54 (m, 1H)

### Example 323: [2-amino-4-(pentafluoro-λ⁶-sulfanyl)phenyl]-[4-[2-(1-methoxy-1-methyl-ethyl)-3H-imidazo[4,5-b]pyridin-7-yl]-1-piperidyl]methanone

Example 323 was prepared following a procedure similar to that of Example 293 using 2-methoxy-2-methyl-propanoic acid in step 1, without MeOH as co-solvent in step 2, and using 2-amino-4-(pentafluoro-λ⁶-sulfanyl)benzoic acid in step 3. LC/MS (m/z, M+H): 520; ¹H NMR (400 MHz, DMSO-d6, 100° C.) δ ppm 1.62 (s, 6H) 1.79-2.08 (m, 4H) 3.05-3.19 (m, 2H) 3.10 (s, 3H) 3.43-3.55 (m, 1H) 4.03-4.24 (m, 2H) 5.46 (br s, 2H) 7.00 (dd, J=8.4, 2.1 Hz, 1H) 7.04 (d, J=5.0 Hz, 1H) 7.23 (br d, J=8.4 Hz, 1H) 7.27 (d, J=2.1 Hz, 1H) 8.19 (brs, 1H) 11.78-13.07 (m, 1H)

### Example 324: [4-[2-(3-hydroxycyclobutyl)-3H-imidazo[4,5-b]pyridin-7-yl]-1-piperidyl]-[4-(pentafluoro-λ⁶-sulfanyl)phenyl]methanone

### Intermediate I: 3-[tert-Butyl(dimethyl)silyl]oxycyclobutanecarboxylic acid

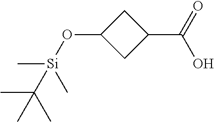

To a solution of 3-hydroxycyclobutanecarboxylic acid (1000 mg, 8.61 mmol) and imidazole (1172 mg, 17.22 mmol) in DMF (4 mL) was added at room temperature portionwise tert-butyl-chloro-dimethyl-silane (1428 mg, 9.47 mmol) and the resulting mixture was stirred at room temperature for 36 hours. After 36 hours, the reaction mixture was transferred in a separating funnel containing water, and extracted twice with AcOEt and Et₂O. The combined organic layers were washed with a 1N HCl solution, dried over magnesium sulfate, filtered and concentrated in vacuo.

The resulting residue was dissolved in 3 mL of MeOH+30 mL of THF, a solution of potassium carbonate (5 g) in water (35 mL) was added, and the whole solution was stirred for 12 hours. Then, the reaction mixture was concentrated in vacuo, and the resulting aqueous layer was extracted with Et₂O. The aqueous layer was adjusted to pH=2 with an aqueous saturated solution of NaHSO₄ (15% in water), extracted twice with ethyl acetate. The combined organic layers were then dried over magnesium sulfate, filtered and concentrated to dryness to give 1 g (50% yield) of pure 3-[tert-butyl(dimethyl)silyl]oxycyclobutanecarboxylic acid as a white solid.

Example 324 was prepared following a procedure similar to that of Example 293 using (cis)-Intermediate I (prepared following a procedure similar to Intermediate I from (cis)-3-hydroxycyclobutanecarboxylic acid) in step 1, 5-6N HCl in iPrOH in step 2, and using 4-(pentafluoro-λ⁶-sulfanyl)benzoic acid in step 3. LC/MS (m/z, M+H): 503; ¹H NMR (400 MHz, DMSO-d6, 100° C.) δ ppm 1.78-2.04 (m, 4H) 2.21-2.36 (m, 2H) 2.58-2.70 (m, 2H) 3.05-3.20 (m, 3H) 3.36-3.58 (m, 1H) 3.95-4.39 (m, 3H) 7.01 (d, J=5 Hz, 1H) 7.66 (d, J=8.5 Hz, 2H) 7.94 (d, J=8.5 Hz, 2H) 8.14 (br d, J=5.0 Hz, 1H) 11.62-12.87 (m, 1H)

### Example 325: [4-[2-[(2S)-4-methylmorpholin-2-yl]-3H-imidazo[4,5-b]pyridin-7-yl]-1-piperidyl]-[4-(pentafluoro-λ⁶-sulfanyl)phenyl]methanone

Example 326 was prepared following a procedure similar to that of Example 314 using [4-[2-[(2S)-morpholin-2-yl]-3H-imidazo[4,5-b]pyridin-7-yl]-1-piperidyl]-[4-(pentafluoro-λ⁶-sulfanyl)phenyl]methanone (Example 316)

### Example 326: (cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone

Example 326 was prepared following a procedure similar to that of Example 293 using (cis)-Intermediate I (prepared following a procedure similar to Intermediate I from (cis)-3-hydroxycyclobutanecarboxylic acid) in step 1, 5-6N HCl in iPrOH in step 2, and using 2-amino-4-(pentafluoro-λ⁶-sulfanyl)benzoic acid in step 3. LC/MS (m/z, M+H): 518; ¹H NMR (400 MHz, DMSO-d6, 100° C.) δ ppm 1.76-2.05 (m, 4H) 2.19-2.35 (m, 2H) 2.56-2.72 (m, 2H) 3.02-3.21 (m, 3H) 3.30-3.56 (m, 1H) 4.00-4.28 (m, 3H) 4.69-5.00 (m, 1H) 5.46 (br s, 2H) 6.93-7.05 (m, 2H) 7.19-7.30 (m, 2H) 8.13 (br s, 1H) 11.61-12.73 (m, 1H)

### Example 327: (cis)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone

### Intermediate II: (cis)-4-[tert-butyl(dimethyl)silyl]oxycyclohexanecarboxylic acid

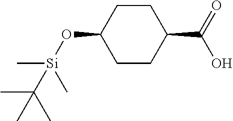 [SMILES: CC(C)(C)[Si](C)(C)O[C@H]1CC[C@@H](C(=O)O)CC1]

To a solution of (cis)-4-hydroxycyclohexanecarboxylic acid (1000 mg, 6.93 mmol) in DMF (7 mL) were successively added at room temperature tert-butyl-chloro-dimethyl-silane (2300 mg, 15.2 mmol) and triethylamine (2.13 mL, 15.26 mmol). The resulting mixture was stirred at room temperature for 12 hours. After 12 hours, the reaction mixture was transferred to a separating funnel containing water, pH was adjusted to pH=4 with a 1 N HCl solution, and the mixture was extracted twice with Et₂O. The combined organic layers were washed with an aqueous saturated solution of NaHCO₃, dried over magnesium sulfate, filtered and concentrated in vacuo.

Then, the resulting residue was dissolved in 1/1 mixture of MeOH and THF and treated with a 5M solution of NaOH (2 mL) for 3 hours. Then, the reaction mixture was concentrated in vacuo, and the resulting aqueous layer was extracted with Et₂O. The aqueous layer was adjusted to pH=2 with a 2N HCl solution, and extracted twice with diethyl ether. The combined organic layers were then dried over magnesium sulfate, filtered and concentrated to dryness to give 1.4 g (78% yield) of pure (cis)-4-[tert-butyl(dimethyl)silyl]oxycyclohexanecarboxylic acid as a colorless oil.

Example 327 was prepared following a procedure similar to that of Example 293 using Intermediate II in step 1, without MeOH as co-solvent in step 2, and using 4-(pentafluoro-λ⁶-sulfanyl)benzoic acid in step 3. LC/MS (m/z, M+H): 531; ¹H NMR (400 MHz, DMSO-d6, 100° C.) δ ppm 1.51-1.66 (m, 2H) 1.67-1.81 (m, 4H) 1.82-2.03 (m, 4H) 2.06-2.21 (m, 2H) 3.14 (br t, J=10.7 Hz, 2H) 3.35-3.52 (m, 1H) 3.77-4.34 (m, 4H) 7.00 (d, J=5.0 Hz, 1H) 7.66 (br d, J=8.4 Hz, 2H) 7.94 (d, J=8.4 Hz, 2H) 8.14 (br d, J=5.0 Hz, 1H) 11.54-12.75 (m, 1H)

### Example 328: [2-amino-4-(pentafluoro-λ⁶-sulfanyl)phenyl]-[4-[2-[(2R)-morpholin-2-yl]-3H-imidazo[4,5-b]pyridin-7-yl]-1-piperidyl]methanone

Example 328 was prepared following a procedure similar to that of step 1 of Example 5 using (R)-4-(tert-butoxycarbonyl)morpholine-2-carboxylic acid and [2-amino-4-(pentafluoro-λ⁶-sulfanyl)phenyl]-[4-(2,3-diamino-4-pyridyl)-1-piperidyl]methanone (prepared by a procedure similar to that of step 1 of Example 364), followed by a procedure similar to that of step 7 of Examples 355 and 356 (with DCM as solvent instead of MeOH). LC/MS (m/z, M+H): 533; ¹H NMR (400 MHz, DMSO-d6, 100° C.) δ ppm 1.78-2.06 (m, 4H) 2.74-3.25 (m, 6H) 3.40-3.55 (m, 1H) 3.62-3.72 (m, 1H) 3.89 (dt, J=11.2, 2.7 Hz, 1H) 4.06-4.23 (m, 2H) 4.69 (dd, J=9.5, 2.9 Hz, 1H) 5.46 (s, 2H) 7.00 (dd, J=8.5, 2.3 Hz, 1H) 7.04 (d, J=5.0 Hz, 1H) 7.24 (d, J=8.5 Hz, 1H) 7.27 (d, J=2.3 Hz, 1H) 8.20 (d, J=5.0 Hz, 1H)

### Example 329: (trans)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone

### Intermediate III: (trans)-4-[tert-butyl(dimethyl)silyl]oxycyclohexanecarboxylic acid

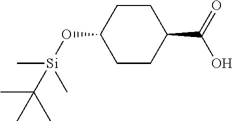 [SMILES: CC(C)(C)[Si](C)(C)O[C@H]1CC[C@H](C(=O)O)CC1]

Intermediate III was prepared following the protocol described for the preparation of intermediate II, using (trans)-4-hydroxycyclohexanecarboxylic acid.

Example 329 was prepared following a procedure similar to that of Example 293 using Intermediate III in step 1, without MeOH as co-solvent in step 2, and using 4-(pentafluoro-λ⁶-sulfanyl)benzoic acid in step 3. LC/MS (m/z, M+H): 531; ¹H NMR (400 MHz, DMSO-d6, 100° C.) δ ppm 1.22-1.40 (m, 2H) 1.69 (qd, J=12.7, 3.1 Hz, 2H) 1.77-2.14 (m, 8H) 2.80 (tt, J=11.8, 3.6 Hz, 1H) 3.13 (br t, J=11.2 Hz, 2H) 3.33-3.57 (m, 2H) 3.89-4.35 (m, 3H) 7.00 (d, J=5.0 Hz, 1H) 7.66 (br d, J=8.4 Hz, 2H) 7.94 (d, J=8.4 Hz, 2H) 8.13 (br s, 1H) 11.67-12.78 (m, 1H)

### Example 330: [4-[2-[(2R)-4-methylmorpholin-2-yl]-3H-imidazo[4,5-b]pyridin-7-yl]-1-piperidyl]-[4-(pentafluoro-λ⁶-sulfanyl)phenyl]methanone

Example 330 was prepared following a procedure similar to that of Example 314 using [4-[2-[(2R)-morpholin-2-yl]-3H-imidazo[4,5-b]pyridin-7-yl]-1-piperidyl]-[4-(pentafluoro-λ⁶-sulfanyl)phenyl]methanone (Example 304). LC/MS (m/z, M+H): 532; ¹H NMR (400 MHz, DMSO-d6, 100° C.) δ ppm 1.73-2.04 (m, 4H) 2.21 (td, J=11.2, 3.4 Hz, 1H) 2.28 (s, 2H) 2.38 (t, J=10.6 Hz, 1H) 2.61-2.74 (m, 1H) 3.00-3.22 (m, 4H) 3.42-3.57 (m, 1H) 3.76 (td, J=11.0, 2.1 Hz, 1H) 3.91-3.99 (m, 1H) 4.01-4.30 (m, 2H) 4.78 (dd, J=9.8, 2.3 Hz, 1H) 7.07 (d, J=5.0 Hz, 1H) 7.66 (d, J=8.5 Hz, 2H) 7.94 (d, J=8.5 Hz, 2H) 8.22 (br d, J=5.0 Hz, 1H) 12.07-12.95 (m, 1H)

### Example 331: (cis)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone

Example 331 was prepared following a procedure similar to that of Example 293 using Intermediate II in step 1, without MeOH as co-solvent in step 2, and using 2-amino-4-(pentafluoro-λ⁶-sulfanyl)benzoic acid in step 3. LC/MS (m/z, M+H): 546; ¹H NMR (400 MHz, DMSO-d6 with acetic acid-d3, 100° C.) δ ppm 1.53-1.69 (m, 2H) 1.70-1.83 (m, 4H) 1.88-2.01 (m, 5H) 2.07-2.21 (m, 2H) 2.92 (tt, J=10.0, 3.6 Hz, 1H) 3.07-3.20 (m, 2H) 3.36-3.49 (m, 1H) 3.81-3.90 (m, 1H) 4.16 (br d, J=11.3 Hz, 2H) 6.97-7.05 (m, 2H) 7.25 (d, J=8.5 Hz, 1H) 7.28 (d, J=2.3 Hz, 1H) 8.15 (d, J=5.0 Hz, 1H)

### Examples 332 and 333: [4-(pentafluoro-λ⁶-sulfanyl)phenyl]-[(4R)-4-(2-tetrahydropyran-4-yl-3H-imidazo[4,5-b]pyridin-7-yl)azepan-1-yl]methanone, Isomers 1 and 2

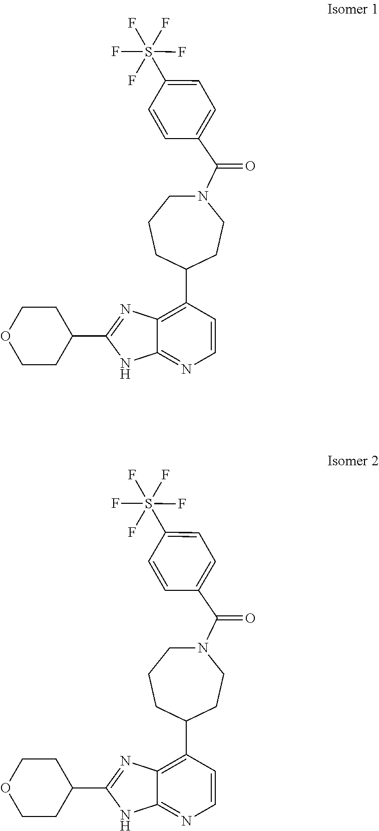 [SMILES: O=C(c1ccc(S(F)(F)(F)(F)F)cc1)N1CCCC(c2ccnc3[nH]c(C4CCOCC4)nc23)CC1 ; O=C(c1ccc(S(F)(F)(F)(F)F)cc1)N1CCCC(c2ccnc3[nH]c(C4CCOCC4)nc23)CC1]

### Step 1: (rac)-tert-butyl 4-(2-tetrahydropyran-4-yl-3H-imidazo[4,5-b]pyridin-7-yl)-2,3,6,7-tetrahydroazepine-1-carboxylate

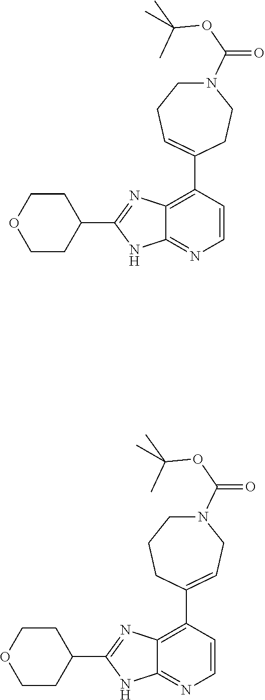 [SMILES: CC(C)(C)OC(=O)N1CCC=C(c2ccnc3[nH]c(C4CCOCC4)nc23)CC1 ; CC(C)(C)OC(=O)N1CC=C(c2ccnc3[nH]c(C4CCOCC4)nc23)CCC1]

Step 1 was performed following the protocol described in step 2 of Examples 339-342, using tert-butyl-4-(4,4,5,5-tetramethyl-1,3,2-dioxaborolan-2-yl)-2,3,6,7-tetrahydro-1H-azepine-1-carboxylate, to give 471 mg (71% yield) of a 50/50 mixture of (rac)-tert-butyl 4-(2-tetrahydropyran-4-yl-3H-imidazo[4,5-b]pyridin-7-yl)-2,3,6,7-tetrahydroazepine-1-carboxylate.

### Step 2: (rac)-tert-butyl 4-(2-tetrahydropyran-4-yl-3H-imidazo[4,5-b]pyridin-7-yl)azepane-1-carboxylate

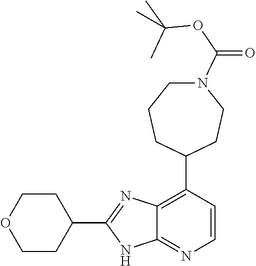 [SMILES: CC(C)(C)OC(=O)N1CCCC(c2ccnc3[nH]c(C4CCOCC4)nc23)CC1]

Step 2 was performed following the same protocol as for Step 3 of Examples 339-342, to give 449 mg (96% yield) of (rac)-tert-butyl 4-(2-tetrahydropyran-4-yl-3H-imidazo[4,5-b]pyridin-7-yl)azepane-1-carboxylate as a white foam.

### Step 3: (rac)-7-(azepan-4-yl)-2-tetrahydropyran-4-yl-3H-imidazo[4,5-b]pyridine;hydrochloride

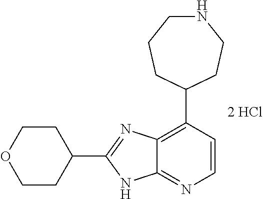 [SMILES: c1cc(C2CCCNCC2)c2nc(C3CCOCC3)[nH]c2n1 ; "2 HCl"]

Step 3 was performed following the protocol described in step 4 of Examples 339-342 to give 308 mg (92% yield) of (rac)-7-(azepan-4-yl)-2-tetrahydropyran-4-yl-3H-imidazo[4,5-b]pyridine;hydrochloride as brown solid.

### Step 4: (rac)-[4-(pentafluoro-λ⁶-sulfanyl)phenyl]-[4-(2-tetrahydropyran-4-yl-3H-imidazo[4,5-b]pyridin-7-yl)azepan-1-yl]methanone

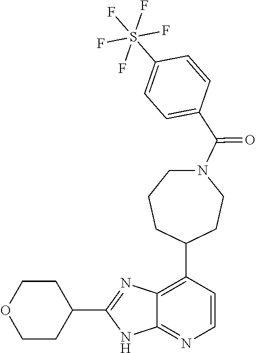 [SMILES: O=C(c1ccc(S(F)(F)(F)(F)F)cc1)N1CCCC(c2ccnc3[nH]c(C4CCOCC4)nc23)CC1]

Step 4 was performed following the protocol described in step 5 of Examples 339-342 using 4-(pentafluorothio)benzoic acid to give 80 mg (67% yield) of (rac)-[4-(pentafluoro-λ⁶-sulfanyl)phenyl]-[4-(2-tetrahydropyran-4-yl-3H-imidazo[4,5-b]pyridin-7-yl)azepan-1-yl]methanone as a white foam.

### Step 5: [4-(pentafluoro-λ⁶-sulfanyl)phenyl]-[4-(2-tetrahydropyran-4-yl-3H-imidazo[4,5-b]pyridin-7-yl)azepan-1-yl]methanone, Isomers 1 and 2

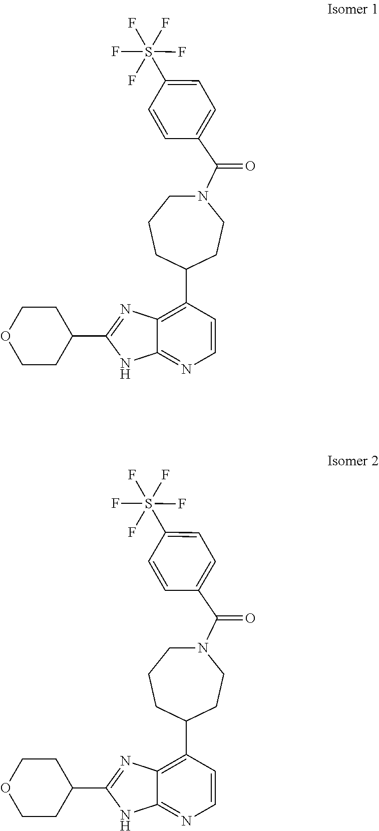 [SMILES: O=C(c1ccc(S(F)(F)(F)(F)F)cc1)N1CCCC(c2ccnc3[nH]c(C4CCOCC4)nc23)CC1 ; O=C(c1ccc(S(F)(F)(F)(F)F)cc1)N1CCCC(c2ccnc3[nH]c(C4CCOCC4)nc23)CC1]

(rac)-[4-(pentafluoro-λ⁶-sulfanyl)phenyl]-[4-(2-tetrahydropyran-4-yl-3H-imidazo[4,5-b]pyridin-7-yl)azepan-1-yl]methanone was separated following Method CS3 (eluting with (n-Heptane 75% EtOH 25%)+0.1% TEA (flow rate 45 mL)) to give 27 mg (38% yield) of [4-(pentafluoro-λ⁶-sulfanyl)phenyl]-[4-(2-tetrahydropyran-4-yl-3H-imidazo[4,5-b]pyridin-7-yl)azepan-1-yl]methanone, Isomer 1 and 30 mg (43% yield) of [4-(pentafluoro-λ⁶-sulfanyl)phenyl]-[4-(2-tetrahydropyran-4-yl-3H-imidazo[4,5-b]pyridin-7-yl)azepan-1-yl]methanone, Isomer 2 as white solids.

Example 332 (Isomer 1): LC/MS (m/z, M+H, Method 1): calc. 531.2, found 531.1; ¹H NMR (400 MHz, DMSO-d6 with acetic acid-d3, 100° C.) δ ppm 1.73-2.24 (m, 10H) 2.91-4.49 (m, 10H) 7.01 (d, J=5.1 Hz, 1H) 7.66 (d, J=8.5 Hz, 2H) 7.92 (d, J=8.6 Hz, 2H) 8.15 (d, J=5.1 Hz, 1H)

Example 333 (Isomer 2): LC/MS (m/z, M+H, Method 1): calc. 531.2, found 531.1; ¹H NMR (400 MHz, DMSO-d6, 100° C.) δ ppm 1.64-2.24 (m, 10H) 3.05-4.07 (m, 10H) 6.99 (d, J=5.1 Hz, 1H) 7.65 (d, J=8.6 Hz, 2H) 7.92 (d, J=8.6 Hz, 2H) 8.12 (br s, 1H) 11.76-12.51 (m, 1H)

### Example 334: [4-[2-[(1R,4R)-2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone

Example 334 was prepared following a procedure similar to that of step 1 of Example 5 using (1R,4R)-5-tert-butoxycarbonyl-2-oxa-5-azabicyclo[2.2.1]heptane-1-carboxylic acid and [4-(2,3-diamino-4-pyridyl)-1-piperidyl]-[4-(pentafluoro-λ⁶-sulfanyl)phenyl]methanone (described in step 1 of Example 408), followed by a procedure similar to that of step 4 Examples 1 and 2 (using HCl 2 M in Et₂O). LC/MS (m/z, M+H): 530; ¹H NMR (400 MHz, DMSO-d6, 100° C.) δ ppm 1.68-2.03 (m, 4H) 2.04-2.18 (m, 2H) 3.06-3.27 (m, 3H) 3.33-3.43 (m, 1H) 3.45-3.58 (m, 1H) 3.75 (br s, 1H) 3.81-4.36 (m, 4H) 7.07 (d, J=5.0 Hz, 1H) 7.65 (br d, J=7.8 Hz, 2H) 7.94 (d, J=8.8 Hz, 2H) 8.15-8.32 (m, 1H) 12.05-12.77 (m, 1H)

### Example 335: (trans)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone

Example 335 was prepared following a procedure similar to that of Example 293 using Intermediate III in step 1, without MeOH as co-solvent in step 2, and using 2-amino-4-(pentafluoro-λ⁶-sulfanyl)benzoic acid in step 3. LC/MS (m/z, M+H): 546; ¹H NMR (400 MHz, DMSO-d6 with Trifluoroacetic acid-D, 100° C.) δ ppm 1.31-1.45 (m, 2H) 1.72-2.09 (m, 8H) 2.12-2.24 (m, 2H) 3.02-3.19 (m, 3H) 3.48-3.65 (m, 2H) 4.21 (br d, J=10.4 Hz, 2H) 7.03 (dd, J=8.5, 2.3 Hz, 1H) 7.24-7.35 (m, 2H) 7.55 (d, J=5.6 Hz, 1H) 8.52 (d, J=5.6 Hz, 1H)

### Example 336: 3-methyl-3-[7-[1-[4-(pentafluoro-λ⁶-sulfanyl)benzoyl]-4-piperidyl]-3H-imidazo[4,5-b]pyridin-2-yl]cyclobutanone

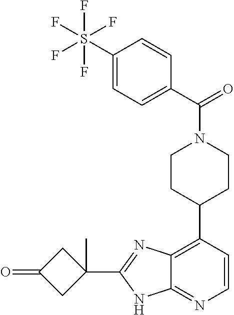 [SMILES: CC1(c2nc3c(C4CCN(C(=O)c5ccc(S(F)(F)(F)(F)F)cc5)CC4)ccnc3[nH]2)CC(=O)C1]

### Step 1: [4-[2-[3-[tert-butyl(dimethyl)silyl]oxy-1-bicyclo[1.1.1]pentanyl]-3H-imidazo[4,5-b]pyridin-7-yl]-1-piperidyl]-[4-(pentafluoro-λ⁶-sulfanyl)phenyl]methanone

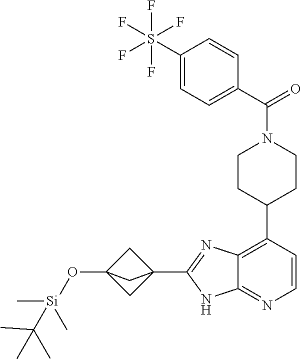 [SMILES: CC(C)(C)[Si](C)(C)OC12CC(c3nc4c(C5CCN(C(=O)c6ccc(S(F)(F)(F)(F)F)cc6)CC5)ccnc4[nH]3)(C1)C2]

Step 1 was performed following the protocol described in step 1 of Example 5 using 3-[tert-butyl(dimethyl)silyl]oxybicyclo[1.1.1]pentane-1-carboxylic acid (Intermediate IV) and [4-(2,3-diamino-4-pyridyl)-1-piperidyl]-[4-(pentafluoro-λ⁶-sulfanyl)phenyl]methanone (described in step 1 of Example 408) to give 85 mg (65% yield) of [4-[2-[3-[tert-butyl(dimethyl)silyl]oxy-1-bicyclo[1.1.1]pentanyl]-3H-imidazo[4,5-b]pyridin-7-yl]-1-piperidyl]-[4-(pentafluoro-λ⁶-sulfanyl)phenyl]methanone as a white solid.

### Step 2: 3-methyl-3-[7-[1-[4-(pentafluoro-λ⁶-sulfanyl)benzoyl]-4-piperidyl]-3H-imidazo[4,5-b]pyridin-2-yl]cyclobutanone

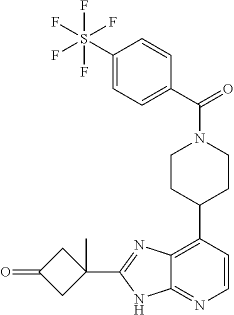 [SMILES: CC1(c2nc3c(C4CCN(C(=O)c5ccc(S(F)(F)(F)(F)F)cc5)CC4)ccnc3[nH]2)CC(=O)C1]

Under argon atmosphere, to a solution of [4-[2-[3-[tert-butyl(dimethyl)silyl]oxy-1-bicyclo[1.1.1]pentanyl]-3H-imidazo[4,5-b]pyridin-7-yl]-1-piperidyl]-[4-(pentafluoro-λ⁶-sulfanyl)phenyl]methanone (85 mg, 0.14 mmol) in THF (4 mL) was added TBAF (0.16 mL, 0.16 mmol, 1 mol/L solution in THF) and the resulting reaction mixture was stirred at room temperature for 12 hours, then concentrated in vacuo. The resulting residue was diluted with AcOEt and water, and transferred to a separating funnel. The organic layer was dried over magnesium sulfate, filtered and concentrated in vacuo. The resulting residue was purified on silica gel (SiO₂ 10 g) eluting with DCM/(MeOH/NH₄OH 90/10) 100/0 to 50/50 to give 26 mg (37% yield) of 3-methyl-3-[7-[1-[4-(pentafluoro-λ⁶-sulfanyl)benzoyl]-4-piperidyl]-3H-imidazo[4,5-b]pyridin-2-yl]cyclobutanone as a white solid. LC/MS (m/z, M+H, Method 1): calc. 515.1, found 515.0; ¹H NMR (400 MHz, DMSO-d6, 100° C.) δ ppm 1.82 (s, 3H) 1.90-2.08 (m, 4H) 2.30-2.30 (m, 1H) 3.09-3.29 (m, 3H) 3.44-3.62 (m, 1H) 3.71-3.85 (m, 2H) 3.90-4.46 (m, 2H) 7.09 (d, J=5.0 Hz, 1H) 7.68 (br d, J=8.4 Hz, 2H) 7.97 (d, J=8.4 Hz, 2H) 8.15-8.33 (m, 1H) 11.89-12.99 (m, 1H)

### Example 337: [4-[2-(1-hydroxy-1-methyl-ethyl)-3H-imidazo[4,5-b]pyridin-7-yl]-1-piperidyl]-[4-(pentafluoro-λ⁶-sulfanyl)phenyl]methanone

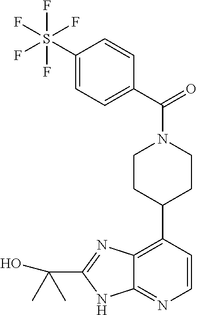 [SMILES: CC(C)(O)c1nc2c(C3CCN(C(=O)c4ccc(S(F)(F)(F)(F)F)cc4)CC3)ccnc2[nH]1]

### Step 1: tert-butyl 4-[2-(1-acetoxy-1-methyl-ethyl)-3H-imidazo[4,5-b]pyridin-7-yl]piperidine-1-carboxylate

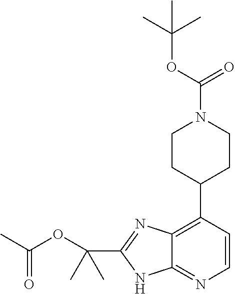 [SMILES: CC(=O)OC(C)(C)c1nc2c(C3CCN(C(=O)OC(C)(C)C)CC3)ccnc2[nH]1]

Step 1 was performed following the protocol described in step 1 of Example 5 using 2-acetoxy-2-methyl-propanoic acid and tert-butyl 4-(2,3-diamino-4-pyridyl)piperidine-1-carboxylate to give 270 mg (49% yield) of tert-butyl 4-[2-(1-acetoxy-1-methyl-ethyl)-3H-imidazo[4,5-b]pyridin-7-yl]piperidine-1-carboxylate as a white foam.

### Step 2: [1-methyl-1-[7-(4-piperidyl)-3H-imidazo[4,5-b]pyridin-2-yl]ethyl]acetate;hydrochloride

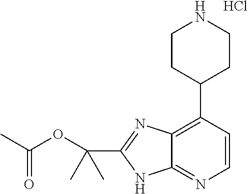 [SMILES: Cl ; CC(=O)OC(C)(C)c1nc2c(C3CCNCC3)ccnc2[nH]1]

Step 2 was performed following the protocol described in step 3 of Example 29 to give 227 mg (100% yield) of [1-methyl-1-[7-(4-piperidyl)-3H-imidazo[4,5-b]pyridin-2-yl]ethyl]acetate;hydrochloride as a white solid.

### Step 3: [1-methyl-1-[7-[1-[4-(pentafluoro-lambda6-sulfanyl)benzoyl]-4-piperidyl]-3H-imidazo[4,5-b]pyridin-2-yl]ethyl]acetate

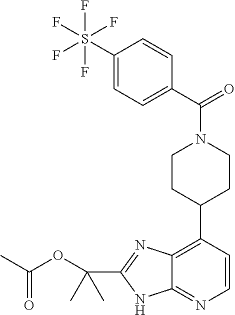 [SMILES: CC(=O)OC(C)(C)c1nc2c(C3CCN(C(=O)c4ccc(S(F)(F)(F)(F)F)cc4)CC3)ccnc2[nH]1]

Step 3 was performed following the protocol described in step 8 of Examples 355 and 356 using 4-(pentafluoro-λ⁶-sulfanyl)benzoic acid to give 157 mg (100% yield) of crude [1-methyl-1-[7-[1-[4-(pentafluoro-lambda6-sulfanyl)benzoyl]-4-piperidyl]-3H-imidazo[4,5-b]pyridin-2-yl]ethyl]acetate which was used in the next step without further purification.

### Step 4: [4-[2-(1-hydroxy-1-methyl-ethyl)-3H-imidazo[4,5-b]pyridin-7-yl]-1-piperidyl]-[4-(pentafluoro-λ⁶-sulfanyl)phenyl]methanone

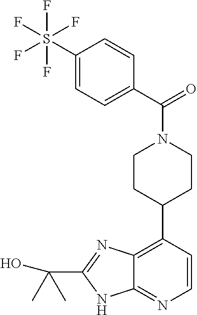 [SMILES: CC(C)(O)c1nc2c(C3CCN(C(=O)c4ccc(S(F)(F)(F)(F)F)cc4)CC3)ccnc2[nH]1]

Under argon atmosphere, to a solution of [1-methyl-1-[7-[1-[4-(pentafluoro-lambda6-sulfanyl)benzoyl]-4-piperidyl]-3H-imidazo[4,5-b]pyridin-2-yl]ethyl]acetate (180 mg, 0.34 mmol) in MeOH (3 mL) was added potassium carbonate (74 mg, 0.51 mmol), and the reaction mixture was stirred at room temperature for 12 hours. After 12 hours, the reaction mixture was concentrated in vacuo, diluted with AcOEt and water, transferred in a separating funnel. The organic layer was then dried over magnesium sulfate, filtered and concentrated in vacuo. The resulting residue was purified on silica gel (SiO₂ 15 g) eluting with DCM/MeOH/NH₄OH 90/10/1 to give 79 mg (47% yield) of [4-[2-(1-hydroxy-1-methyl-ethyl)-3H-imidazo[4,5-b]pyridin-7-yl]-1-piperidyl]-[4-(pentafluoro-λ⁶-sulfanyl)phenyl]methanone as a white solid. LC/MS (m/z, M+H, Method 1): calc. 491.1, found 491.0.

### Example 338: [2-amino-4-(pentafluoro-λ⁶-sulfanyl)phenyl]-[4-[2-(1-hydroxy-1-methyl-ethyl)-3H-imidazo[4,5-b]pyridin-7-yl]-1-piperidyl]methanone

Example 338 was prepared following a procedure similar to that of Example 337 using 2-amino-4-(pentafluoro-λ⁶-sulfanyl)benzoic acid, instead of 4-(pentafluoro-λ⁶-sulfanyl)benzoic acid, in step 3. LC/MS (m/z, M+H): 506; ¹H NMR (400 MHz, DMSO-d6, 100° C.) δ ppm 1.60 (s, 6H) 1.80-2.03 (m, 4H) 3.05-3.19 (m, 2H) 3.41-3.56 (m, 1H) 4.03-4.25 (m, 2H) 4.99-5.23 (m, 1H) 5.45 (s, 2H) 6.94-7.05 (m, 2H) 7.23 (d, J=8.5 Hz, 1H) 7.27 (d, J=2.3 Hz, 1H) 8.17 (br d, J=5.0 Hz, 1H) 11.66-12.63 (m, 1H)

### Examples 339-342: [(cis)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone, Isomers 1 and 2, and [(trans)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone Isomers 1 and 2

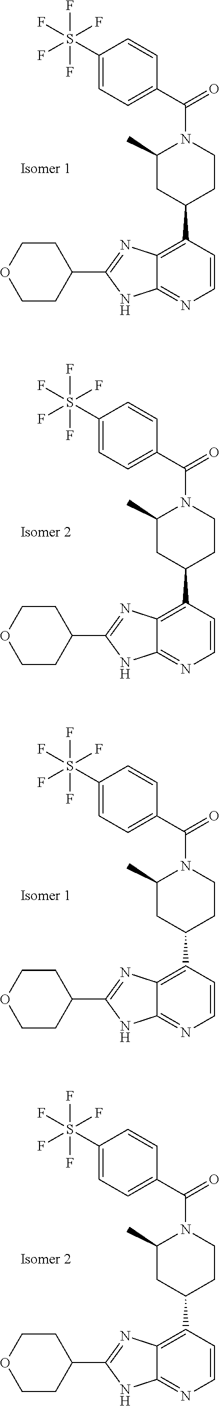 [SMILES: C[C@@H]1C[C@H](c2ccnc3[nH]c(C4CCOCC4)nc23)CCN1C(=O)c1ccc(S(F)(F)(F)(F)F)cc1 ; C[C@@H]1C[C@H](c2ccnc3[nH]c(C4CCOCC4)nc23)CCN1C(=O)c1ccc(S(F)(F)(F)(F)F)cc1 ; C[C@@H]1C[C@@H](c2ccnc3[nH]c(C4CCOCC4)nc23)CCN1C(=O)c1ccc(S(F)(F)(F)(F)F)cc1 ; C[C@@H]1C[C@@H](c2ccnc3[nH]c(C4CCOCC4)nc23)CCN1C(=O)c1ccc(S(F)(F)(F)(F)F)cc1]

### Step 1: 7-chloro-2-tetrahydropyran-4-yl-3H-imidazo[4,5-b]pyridine

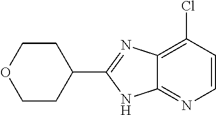 [SMILES: Clc1ccnc2[nH]c(C3CCOCC3)nc12]

Step 1 was performed following the protocol described in step 1 of Example 15 using 4-chloropyridine-2,3-diamine and tetrahydro-2H-pyran-4-carboxylic acid to give 1.15 g (72% yield) of 7-chloro-2-tetrahydropyran-4-yl-3H-imidazo[4,5-b]pyridine as a white solid.

### Step 2: tert-butyl 2-methyl-4-(2-tetrahydropyran-4-yl-3H-imidazo[4,5-b]pyridin-7-yl)-3,6-dihydro-2H-pyridine-1-carboxylate and tert-butyl 6-methyl-4-(2-tetrahydropyran-4-yl-3H-imidazo[4,5-b]pyridin-7-yl)-3,6-dihydro-2H-pyridine-1-carboxylate

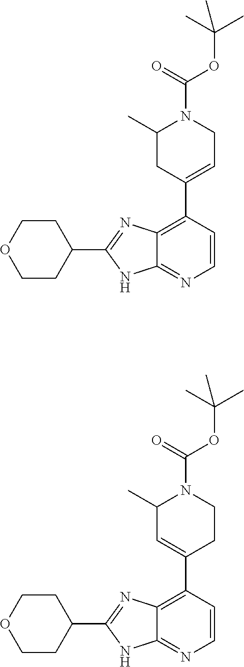 [SMILES: CC1CC(c2ccnc3[nH]c(C4CCOCC4)nc23)=CCN1C(=O)OC(C)(C)C ; CC1C=C(c2ccnc3[nH]c(C4CCOCC4)nc23)CCN1C(=O)OC(C)(C)C]

In a 2-5 mL microwave vial, to a solution of 7-chloro-2-tetrahydropyran-4-yl-3H-imidazo[4,5-b]pyridine (223 mg, 0.94 mmol) in dioxane (4.5 mL) and water (0.45 mL) were successively added Cs₂CO₃ (763 mg, 2.34 mmol), Pd(dppf)Cl₂·DCM (76 mg, 0.09 mmol) and finally tert-butyl-2-methyl-4-(4,4,5,5-tetramethyl-1,3,2-dioxaborolan-2-yl)-3,6-dihydropyridine-1(2H)-carboxylate (485 mg, 1.5 mmol). The resulting mixture was bubbled with argon for 10 minutes. Then the vial was sealed and submitted to microwave for 30 minutes at 160° C. After 30 minutes, the reaction mixture was diluted with AcOEt, transferred to a separating funnel, and washed with brine. The organic layer was then dried over magnesium sulfate, filtered and concentrated in vacuo. The resulting residue was purified on silica gel (SiO₂ 200 g) eluting with DCM/i-PrOH 96/4 to 94/6 to give 280 mg (75% yield) of a mixture of tert-butyl 2-methyl-4-(2-tetrahydropyran-4-yl-3H-imidazo[4,5-b]pyridin-7-yl)-3,6-dihydro-2H-pyridine-1-carboxylate and tert-butyl 6-methyl-4-(2-tetrahydropyran-4-yl-3H-imidazo[4,5-b]pyridin-7-yl)-3,6-dihydro-2H-pyridine-1-carboxylate as a white solid.

### Step 3: tert-butyl 2-methyl-4-(2-tetrahydropyran-4-yl-3H-imidazo[4,5-b]pyridin-7-yl)piperidine-1-carboxylate

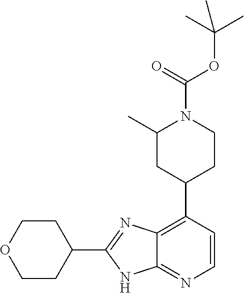 [SMILES: CC1CC(c2ccnc3[nH]c(C4CCOCC4)nc23)CCN1C(=O)OC(C)(C)C]

To a solution of a mixture of tert-butyl 2-methyl-4-(2-tetrahydropyran-4-yl-3H-imidazo[4,5-b]pyridin-7-yl)-3,6-dihydro-2H-pyridine-1-carboxylate and tert-butyl 6-methyl-4-(2-tetrahydropyran-4-yl-3H-imidazo[4,5-b]pyridin-7-yl)-3,6-dihydro-2H-pyridine-1-carboxylate (853 mg, 2.14 mmol) in EtOH (30 mL) and THF (15 mL), was added Pd/C (228 mg, 0.21 mmol) and the mixture was stirred under H₂ atmosphere (2 bars) at 50° C. for 12 hours. An additional 228 mg of Pd/C (228 mg, 0.21 mmol) was added and the mixture was stirred under H₂ atmosphere (4 bars) at 50° C. for 12 hours. Then, the mixture was filtered through Celite, and the solvent was concentrated in vacuo. A further additional 228 mg of Pd/C (228 mg, 0.21 mmol) was added and the mixture was stirred under H₂ atmosphere (4 bars) at 50° C. for 48 hours. The mixture was filtered through Celite, and the filtrate was concentrated in vacuo. The crude was dissolved in a mixture of THF/EtOH (15 ml/30 ml), and it was added 2027 mg of Pd/C. The resulting mixture was stirred under H₂ atmosphere (4 bars) at 50° C. for 48 hours. The mixture was filtered through Celite, then the filtrate was concentrated in vacuo to give 729 mg of crude tert-butyl 2-methyl-4-(2-tetrahydropyran-4-yl-3H-imidazo[4,5-b]pyridin-7-yl)piperidine-1-carboxylate as a white foam which was used in the next step without further purification.

### Step 4: 7-(2-methyl-4-piperidyl)-2-tetrahydropyran-4-yl-3H-imidazo[4,5-b]pyridine;hydrochloride

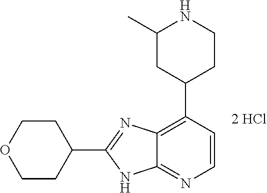 [SMILES: CC1CC(c2ccnc3[nH]c(C4CCOCC4)nc23)CCN1]

Step 4 was performed following the protocol described in step 3 of Example 29 to give 643 mg (100% yield) of 7-(2-methyl-4-piperidyl)-2-tetrahydropyran-4-yl-3H-imidazo[4,5-b]pyridine;hydrochloride as a white solid.

### Step 5: [2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone

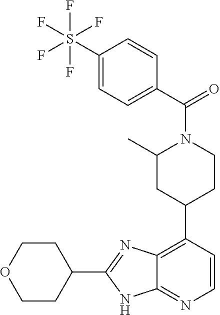 [SMILES: CC1CC(c2ccnc3[nH]c(C4CCOCC4)nc23)CCN1C(=O)c1ccc(S(F)(F)(F)(F)F)cc1]

Step 5 was performed following the protocol described in step 8 of Examples 355 and 356 using 4-(pentafluorothio)benzoic acid to give 157 mg (57% yield) of [2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone as a white foam.

### Step 6: [(cis)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone, Isomers 1 and 2, and [(trans)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone Isomers 1 and 2

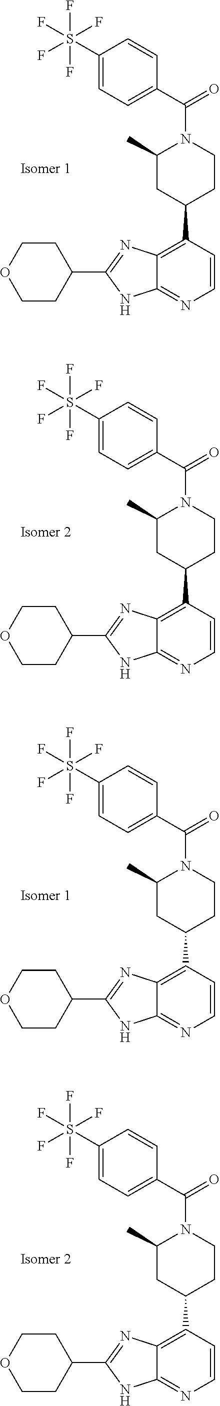 [SMILES: C[C@@H]1C[C@H](c2ccnc3[nH]c(C4CCOCC4)nc23)CCN1C(=O)c1ccc(S(F)(F)(F)(F)F)cc1 ; C[C@@H]1C[C@H](c2ccnc3[nH]c(C4CCOCC4)nc23)CCN1C(=O)c1ccc(S(F)(F)(F)(F)F)cc1 ; C[C@@H]1C[C@@H](c2ccnc3[nH]c(C4CCOCC4)nc23)CCN1C(=O)c1ccc(S(F)(F)(F)(F)F)cc1 ; C[C@@H]1C[C@@H](c2ccnc3[nH]c(C4CCOCC4)nc23)CCN1C(=O)c1ccc(S(F)(F)(F)(F)F)cc1]

The four isomers of [2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone were separated using first Method CS8 (with Whelk 01 SS column (5 μm, 250×30 mm), eluting with (n-Heptane 80%, EtOH/MeOH 15%/5%)+TEA 0.1% (flow rate 45 mL)), followed by Method CS3 (with n-heptane 90/EtOH 10)+0.1% TEA (flow rate 45 mL/min)), to give (cis)-[2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone, Isomers 1 and 2, and (trans)-[2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone, Isomers 1 and 2, as white solids in the quantities recited below.

Example 339 (cis-Isomer 1): 18 mg (11% yield); LC/MS (m/z, M+H, Method 1): calc. 531.2, found 531.0; ¹H NMR (400 MHz, DMSO-d6, 100° C.) δ ppm 1.26 (d, J=6.4 Hz, 3H), 1.88-2.27 (m, 8H), 3.18 (tt, J=10.0, 5.2 Hz, 1H), 3.39-3.71 (m, 5H), 3.98 (dt, J=11.4, 3.4 Hz, 2H), 4.20-4.33 (m, 1H), 7.06 (d, J=5.0 Hz, 1H), 7.68 (br d, J=8.6 Hz, 2H), 7.97 (d, J=8.6 Hz, 2H), 8.18 (br d, J=5.0 Hz, 1H), 12.11-12.65 (m, 1H)

Example 340 (cis-Isomer 2): 42 mg (27% yield); LC/MS (m/z, M+H, Method 1): calc. 531.2, found 531.0; ¹H NMR (400 MHz, DMSO-d6, 100° C.) δ ppm 1.37 (d, J=6.9 Hz, 3H), 1.63-2.24 (m, 8H), 3.06-3.37 (m, 2H), 3.41-3.59 (m, 2H), 3.57-3.76 (m, 1H), 3.77-5.30 (m, 4H), 7.01 (d, J=5.0 Hz, 1H), 7.63 (br d, J=8.8 Hz, 2H), 7.94 (d, J=8.8 Hz, 2H), 8.10-8.32 (m, 1H), 11.95-12.61 (m, 1H)

Example 341 (trans-Isomer 1): 39 mg (25% yield); LC/MS (m/z, M+H, Method 1): calc. 531.2, found 531.1; ¹H NMR (400 MHz, DMSO-d6, 100° C.) δ ppm 1.26 (d, J=6.5 Hz, 3H), 1.88-2.26 (m, 8H), 3.18 (tt, J=10.0, 5.1 Hz, 1H), 3.36-3.68 (m, 5H), 3.99 (dt, J=11.4, 3.4 Hz, 2H), 4.26 (dq, J=14.0, 7.0 Hz, 1H), 7.07 (d, J=5.1 Hz, 1H), 7.68 (br d, J=8.6 Hz, 2H), 7.97 (d, J=8.6 Hz, 2H), 8.18 (br d, J=5.1 Hz, 1H), 11.83-12.78 (m, 1H)

Example 342 (trans-Isomer 2): 29 mg (18% yield); LC/MS (m/z, M+H, Method 1): calc. 531.2, found 531.01; ¹H NMR (400 MHz, DMSO-d6, 100° C.) δ ppm 1.40 (d, J=6.9 Hz, 3H), 1.77-2.25 (m, 8H), 3.10-3.44 (m, 2H), 3.48-3.59 (m, 3H), 3.64-3.83 (m, 1H), 3.90-4.30 (m, 3H), 7.04 (d, J=5.0 Hz, 1H), 7.66 (br d, J=8.8 Hz, 2H), 7.97 (d, J=8.8 Hz, 2H), 8.13-8.44 (m, 1H), 12.05-12.64 (m, 1H)

### Example 343: [2-amino-4-(pentafluoro-λ⁶-sulfanyl)phenyl]-[4-[2-[(1R,4R)-2-oxa-5-azabicyclo[2.2.1]heptan-1-yl]-3H-imidazo[4,5-b]pyridin-7-yl]-1-piperidyl]methanone

Example 343 was prepared by a procedure similar to that of step 1 of Example 5 using (1R,4R)-5-tert-butoxycarbonyl-2-oxa-5-azabicyclo[2.2.1]heptane-1-carboxylic acid and [2-amino-4-(pentafluoro-λ⁶-sulfanyl)phenyl]-[4-(2,3-diamino-4-pyridyl)-1-piperidyl]methanone (prepared according to step 1 of Example 408 with 2-amino-4-(pentafluoro-λ⁶-sulfanyl)benzoic acid), followed by a procedure similar to that of step 4 of Examples 1 and 2 (using HCl 2 M in Et₂O). LC/MS (m/z, M+H): 545; ¹H NMR (400 MHz, DMSO-d6, 100° C.) δ ppm 1.61-2.02 (m, 4H) 2.04-2.18 (m, 2H) 3.04-3.18 (m, 3H) 3.23 (br d, J=10.0 Hz, 1H) 3.40 (br d, J=10.1 Hz, 1H) 3.49 (br s, 1H) 3.78 (br s, 1H) 3.82-3.93 (m, 1H) 3.94-4.04 (m, 1H) 4.13 (br d, J=6.1 Hz, 2H) 5.46 (br s, 2H) 7.00 (dd, J=8.5, 2.1 Hz, 1H) 7.06 (d, J=5.0 Hz, 1H) 7.23 (br d, J=8.5 Hz, 1H) 7.27 (d, J=2.1 Hz, 1H) 8.06-8.35 (m, 1H)

### Example 344: [2-amino-4-(trifluoromethoxy)phenyl]-[4-fluoro-4-(6-fluoro-2-tetrahydropyran-4-yl-3H-imidazo[4,5-b]pyridin-7-yl)-1-piperidyl]methanone

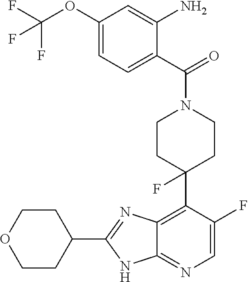 [SMILES: Nc1cc(OC(F)(F)F)ccc1C(=O)N1CCC(F)(c2c(F)cnc3[nH]c(C4CCOCC4)nc23)CC1]

### Step 1: 1-tert-butyl 04-(1,3-dioxoisoindolin-2-yl) 4-fluoropiperidine-1,4-dicarboxylate

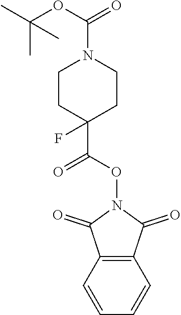 [SMILES: CC(C)(C)OC(=O)N1CCC(F)(C(=O)ON2C(=O)c3ccccc3C2=O)CC1]

To a solution of 1-Boc-4-fluoro-4-piperidinecarboxylic acid (500 mg, 2.02 mmol), N-hydroxyphthalimide (365 mg, 2.23 mmol) and 4-dimethylaminopyridine (25 mg, 0.20 mmol) in dichloromethane (6 mL) was added N,N′-diisopropylcarbodiimide (400 μL, 2.60 mmol) dropwise. The resulting reaction mixture was then stirred at room temperature. After 2 hours, the reaction mixture was filtered through a plug of silica gel, and washed with 50 mL of dichloromethane. The filtrate was concentrated under vacuum to give 574 mg (72% yield) of 01-tert-butyl 04-(1,3-dioxoisoindolin-2-yl) 4-fluoropiperidine-1,4-dicarboxylate as a white solid. LC/MS (m/z, M+H-tBu): 337

### Step 2: tert-butyl-4-fluoro-4-[6-fluoro-2-tetrahydropyran-4-yl-3-(2-trimethylsilylethoxymethyl)imidazo[4,5-b]pyridin-7-yl]piperidine-1-carboxylate

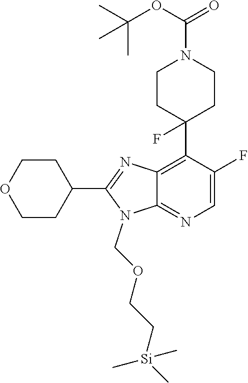 [SMILES: CC(C)(C)OC(=O)N1CCC(F)(c2c(F)cnc3c2nc(C2CCOCC2)n3COCC[Si](C)(C)C)CC1]

In a 4 mL photochemistry vial, were placed 01-tert-butyl 04-(1,3-dioxoisoindolin-2-yl) 4-fluoropiperidine-1,4-dicarboxylate (570 mg, 1.45 mmol), 2-[(6-fluoro-2-tetrahydropyran-4-yl-imidazo[4,5-b]pyridin-3-yl)methoxy]ethyl-trimethyl-silane (280 mg, 0.80 mmol), sodium iodide (120 mg, 0.80 mmol), triphenylphosphine (210 mg, 0.80 mmol) and (R)-(−)-1,1′-binaphthyl-2,2′-diyl hydrogenphosphate (30 mg, 0.09 mmol). The vial was evacuated and filled with argon (three times) and then 2.5 mL of degassed dioxane was added. The reaction mixture was stirred under irradiation with blue LEDs (450 nm, maintained at approximately room temperature by a desk fan). After 4 hours, the reaction mixture was diluted with an aqueous solution of NaOH (0.1N). The aqueous phase was extracted twice with EtOAc and the combined organic phases were dried over magnesium sulfate, filtered and concentrated in vacuo. The resulting residue was purified by flash chromatography (SiO₂ 12 g) eluting with cyclohexane/EtOAc 80/20 to 70/30. The crude product was treated on SCX Cartridge to give 38 mg (6% yield) of tert-butyl-4-fluoro-4-[6-fluoro-2-tetrahydropyran-4-yl-3-(2-trimethylsilylethoxymethyl)imidazo[4,5-b]pyridin-7-yl]piperidine-1-carboxylate was obtained as a colorless wax. LC/MS (m/z, M+H): 553

### Step 3: 6-fluoro-7-(4-fluoro-4-piperidyl)-2-tetrahydropyran-4-yl-3H-imidazo[4,5-b]pyridine;hydrochloride

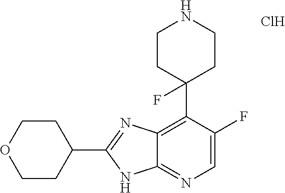 [SMILES: Fc1cnc2[nH]c(C3CCOCC3)nc2c1C1(F)CCNCC1 ; Cl]

To a solution of tert-butyl 4-fluoro-4-[6-fluoro-2-tetrahydropyran-4-yl-3-(2-trimethylsilylethoxymethyl)imidazo[4,5-b]pyridin-7-yl]piperidine-1-carboxylate (37 mg, 0.07 mmol) in 2 mL of MeOH was added a solution of HCl (4N in dioxane, 800 μL, 3.20 mmol). The resulting solution was stirred overnight and then concentrated to dryness. The residue (48 mg) was used without further purification. LC/MS (m/z, M+H): 323

### Step 4: [2-amino-4-(trifluoromethoxy)phenyl]-[4-fluoro-4-(6-fluoro-2-tetrahydropyran-4-yl-3H-imidazo[4,5-b]pyridin-7-yl)-1-piperidyl]methanone

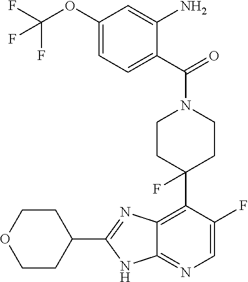 [SMILES: Nc1cc(OC(F)(F)F)ccc1C(=O)N1CCC(F)(c2c(F)cnc3[nH]c(C4CCOCC4)nc23)CC1]

To a solution of 6-fluoro-7-(4-fluoro-4-piperidyl)-2-tetrahydropyran-4-yl-3H-imidazo[4,5-b]pyridine;hydrochloride (48 mg, 0.13 mmol) and 2-amino-4-(trifluoromethoxy)benzoic acid (35 mg, 0.16 mmol) in DMF (2 mL) were added triethylamine (125 μL, 0.90 mmol) and TBTU (50 mg, 0.15 mmol). The resulting mixture was stirred at room temperature for 30 minutes, then diluted with water, transferred to a separating funnel, and extracted twice with AcOEt. The combined organic layers were then dried over magnesium sulfate, filtered and concentrated in vacuo. The resulting residue was purified by flash chromatography (SiO₂ 12 g) eluting with DCM/MeOH 100/0 to 95/5 to give 23 mg (32% yield) of [2-amino-4-(trifluoromethoxy)phenyl]-[4-fluoro-4-(6-fluoro-2-tetrahydropyran-4-yl-3H-imidazo[4,5-b]pyridin-7-yl)-1-piperidyl]methanone as a yellow solid. LC/MS (m/z, M+H, Method 1): calc. 526.2, found 526.0; ¹H NMR (400 MHz, DMSO-d6 with acetic acid-d3, 100° C.) δ ppm 1.88-2.02 (m, 4H) 2.25 (brt, J=11.9 Hz, 2H) 2.56-2.92 (m, 2H) 3.14-3.27 (m, 1H) 3.37 (br t, J=11.5 Hz, 2H) 3.44-3.59 (m, 2H) 3.96 (dt, J=11.5, 3.4 Hz, 2H) 4.08 (br d, J=12.6 Hz, 2H) 6.45-6.54 (m, 1H) 6.71 (br s, 1H) 7.17 (d, J=8.4 Hz, 1H) 8.20 (d, J=4.4 Hz, 1H)

### Example 345: [4-[2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone

### Intermediate IV: 3-[tert-butyl(dimethyl)silyl]oxybicyclo[1.1.1]pentane-1-carboxylic acid

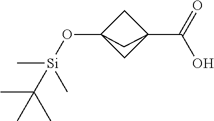 [SMILES: CC(C)(C)[Si](C)(C)OC12CC(C(=O)O)(C1)C2]

To a solution of 3-hydroxybicyclo[1.1.1]pentane-1-carboxylic acid (500 mg, 3.90 mmol), 4-dimethylaminopyridine (95 mg, 0.78 mmol) and imidazole (318 mg, 4.68 mmol) in DCM (10 mL) was added tert-butyl-chloro-dimethyl-silane (647 mg, 4.29 mmol). The resulting mixture was stirred for 12 hours at room temperature, and washed with water. The organic layer was dried over magnesium sulfate, filtered and concentrated to dryness to give 650 mg (69% yield) of crude 3-[tert-butyl(dimethyl)silyl]oxybicyclo[1.1.1]pentane-1-carboxylic acid which was used without further purification.

Example 345 was prepared following a procedure similar to that of Example 293 using Intermediate IV in step 1, without MeOH as co-solvent in step 2, and using 4-(pentafluoro-λ⁶-sulfanyl)benzoic acid in step 3. LC/MS (m/z, M+H): 515; ¹H NMR (400 MHz, DMSO-d6, 30° C.) δ ppm 1.69-2.02 (m, 4H) 2.25 (br s, 6H) 2.88-3.10 (m, 1H) 3.20-3.39 (m hidden, 1H) 3.46-3.67 (m, 2H) 4.57-4.75 (m, 1H) 6.48 (br s, 1H) 7.10 (d, J=5.0 Hz, 1H) 7.69 (br d, J=8.4 Hz, 2H) 8.00 (d, J=8.4 Hz, 2H) 8.11-8.29 (m, 1H) 12.25-13.25 (m, 1H)

### Example 346: [2-amino-4-(pentafluoro-λ⁶-sulfanyl)phenyl]-[4-[2-(3-hydroxy-1-bicyclo[1.1.1]pentanyl)-3H-imidazo[4,5-b]pyridin-7-yl]-1-piperidyl]methanone

Example 346 was prepared following a procedure similar to that of Example 293 using Intermediate IV in step 1, without MeOH as co-solvent in step 2, and using 2-amino-4-(pentafluoro-λ⁶-sulfanyl)benzoic acid in step 3. LC/MS (m/z, M+H): 530; ¹H NMR (400 MHz, DMSO-d6, 100° C.) δ ppm 1.77-2.01 (m, 4H) 2.25 (s, 6H) 3.04-3.18 (m, 2H) 3.30-3.58 (m, 1H) 4.02-4.24 (m, 2H) 5.45 (br s, 2H) 6.09 (br s, 1H) 6.95-7.06 (m, 2H) 7.23 (br d, J=8.4 Hz, 1H) 7.27 (d, J=2.1 Hz, 1H) 8.07-8.26 (m, 1H) 11.69-12.97 (m, 1H)

### Example 347: (rac)-[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone

Example 347 was prepared following a procedure similar to that of steps 1-3 of Examples 332 and 333 using tert-butyl 3-(4,4,5,5-tetramethyl-1,3,2-dioxaborolan-2-yl)-2,5-dihydro-1H-pyrrole-1-carboxylate in step 1, followed by a procedure similar to that of step 8 of Examples 355 and 356 using 2-amino-4-(pentafluoro-λ⁶-sulfanyl)benzoic acid. LC/MS (m/z, M+H): 518; ¹H NMR (400 MHz, DMSO-d6, 100° C.) δ ppm 1.80-2.01 (m, 4H) 2.15-2.45 (m, 2H) 3.13 (tt, J=10.2, 5.0 Hz, 1H) 3.48 (td, J=10.9, 3.4 Hz, 2H) 3.54-3.80 (m, 3H) 3.83-4.05 (m, 4H) 5.63 (br s, 2H) 6.96 (dd, J=8.4, 2.2 Hz, 1H) 7.03 (d, J=5.0 Hz, 1H) 7.25 (d, J=2.2 Hz, 1H) 7.30 (d, J=8.4 Hz, 1H) 8.16 (d, J=5.0 Hz, 1H) 12.21-12.59 (m, 1H)

### Example 348: (rac)-[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone

Example 348 was prepared following a procedure similar to that of steps 1-3 of Examples 332 and 333 using tert-butyl 3-(4,4,5,5-tetramethyl-1,3,2-dioxaborolan-2-yl)-2,5-dihydro-1H-pyrrole-1-carboxylate in step 1, followed by a procedure similar to that of step 8 of Examples 355 and 356 using 4-(pentafluorothio)benzoic acid. LC/MS (m/z, M+H): 503; ¹H NMR (400 MHz, DMSO-d6, 100° C.) δ ppm 1.76-2.03 (m, 4H) 2.18-2.45 (m, 2H) 3.05-3.20 (m, 1H) 3.34-4.16 (m, 9H) 7.04 (br d, J=4.8 Hz, 1H) 7.74 (br d, J=8.4 Hz, 2H) 7.92 (br d, J=8.4 Hz, 2H) 8.11-8.20 (m, 1H) 12.25-12.64 (m, 1H)

### Example 349: [2-hydroxy-4-(pentafluoro-λ⁶-sulfanyl)phenyl]-[4-(2-tetrahydropyran-4-yl-3H-imidazo[4,5-b]pyridin-7-yl)-1-piperidyl]methanone

Example 349 was prepared following a procedure similar to that of Example 131 using 2-hydroxy-4-(pentafluoro-λ⁶-sulfanyl)benzoic acid. LC/MS (m/z, M+H): 533; ¹H NMR (400 MHz, DMSO-d6 with acetic acid-d3, 100° C.) δ ppm 1.77-2.05 (m partially hidden, 8H), 3.01-3.21 (m, 3H), 3.37-3.56 (m, 3H), 3.87-4.28 (m, 4H), 6.99 (d, J=5.1 Hz, 1H), 7.29-7.43 (m, 3H), 8.16 (d, J=5.1 Hz, 1H)

### Example 350: [2-amino-4-(pentafluoro-λ⁶-sulfanyl)phenyl]-[4-(6-fluoro-2-tetrahydropyran-4-yl-3H-imidazo[4,5-b]pyridin-7-yl)-1-piperidyl]methanone

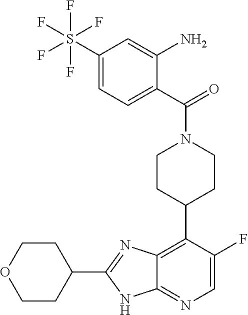 [SMILES: Nc1cc(S(F)(F)(F)(F)F)ccc1C(=O)N1CCC(c2c(F)cnc3[nH]c(C4CCOCC4)nc23)CC1]

### Step 1: 6-fluoro-2-tetrahydropyran-4-yl-3H-imidazo[4,5-b]pyridine

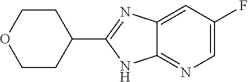 [SMILES: Fc1cnc2[nH]c(C3CCOCC3)nc2c1]

In a microwave vial, to a solution of tetrahydropyran-4-carbaldehyde (275 mg, 2.4 mmol) in DMF (2.5 mL) was added 5-fluoropyridine-2,3-diamine (300 mg, 2.36 mmol). Then, sodium metabisulfite (590 mg, 3.10 mmol) was added, the vial was sealed and submitted to microwave at 125° C. for 45 minutes. After 45 minutes, the reaction mixture was concentrated in vacuo and the resulting residue was purified on silica gel (SiO₂ 40 g) eluting with DCM/MeOH 95/5 to give 386 mg (73% yield) of 6-fluoro-2-tetrahydropyran-4-yl-3H-imidazo[4,5-b]pyridine.

### Step 2: 6-fluoro-4-oxido-2-tetrahydropyran-4-yl-3H-imidazo[4,5-b]pyridin-4-ium

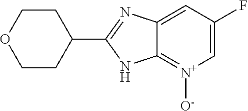 [SMILES: [O-][n+]1cc(F)cc2nc(C3CCOCC3)[nH]c21]

To a solution of 6-fluoro-2-tetrahydropyran-4-yl-3H-imidazo[4,5-b]pyridine (380 mg, 1.72 mmol) in acetic acid (15 mL), was added portionwise 3-chloroperoxybenzoic acid (1.15 g, 6.66 mmol) and the reaction mixture was stirred for 12 hours at room temperature. After 12 hours, the reaction mixture was filtered on SGX cartridge (10 g) and washed first with MeOH, then eluted with a 2N solution of ammonia in MeOH. The collected fractions were concentrated in vacuo to give 335 mg (82% yield) of 6-fluoro-4-oxido-2-tetrahydropyran-4-yl-3H-imidazo[4,5-b]pyridin-4-ium as a yellow powder.

### Step 3: 7-chloro-6-fluoro-2-tetrahydropyran-4-yl-3H-imidazo[4,5-b]pyridine

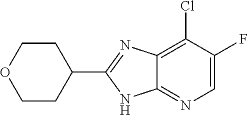 [SMILES: Fc1cnc2[nH]c(C3CCOCC3)nc2c1Cl]

In a microwave vial, to 6-fluoro-4-oxido-2-tetrahydropyran-4-yl-3H-imidazo[4,5-b]pyridin-4-ium (330 mg, 1.39 mmol) was added phosphorus(V) oxychloride (10 mL, 107.3 mmol). The vial was sealed and submitted to microwave at 90° C. for 10 minutes. After 10 minutes, the vial as cooled down to room temperature and the reaction mixture was poured carefully onto a cold saturated aqueous solution of NaHCO₃ (100 mL). The pH was adjusted to 8-9 with solid NaHCO₃. The aqueous layer was extracted with DCM (100 mL). The organic layer was filtered on a liquid-liquid hydrophobic phase separation cartridge (hydrophobic Radleys cartridge, 70 mL). The resulting filtrate was concentrated to dryness to give 345 mg (78% yield) of crude 7-chloro-6-fluoro-2-tetrahydropyran-4-yl-3H-imidazo[4,5-b]pyridine which was used in the next step without further purification.

### Step 4: 6-fluoro-7-iodo-2-tetrahydropyran-4-yl-3H-imidazo[4,5-b]pyridine

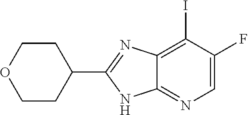 [SMILES: Fc1cnc2[nH]c(C3CCOCC3)nc2c1I]

To a solution of 7-chloro-6-fluoro-2-tetrahydropyran-4-yl-3H-imidazo[4,5-b]pyridine (340 mg, 1.33 mmol) in THF (6 mL) was added 1 mL of a 2 M solution of HCl in diethyl ether. The reaction mixture was stirred for 2 minutes and concentrated to dryness in vacuo. The resulting white powder was solubilized in 12 mL of MeCN and transferred to a microwave vial containing sodium iodide (3 g, 20 mmol). The vial was sealed and submitted to microwave at 160° C. for 30 minutes. The reaction mixture was then poured onto an aqueous saturated solution of NaHCO₃ and Na₂S₂O₃ (50 mL). The whole was stirred for 5 minutes, and filtered to give 402 mg (87% yield) of 6-fluoro-7-iodo-2-tetrahydropyran-4-yl-3H-imidazo[4,5-b]pyridine as a white powder.

### Step 5: 2-[(6-fluoro-7-iodo-2-tetrahydropyran-4-yl-imidazo[4,5-b]pyridin-3-yl)methoxy]ethyl-trimethyl-silane

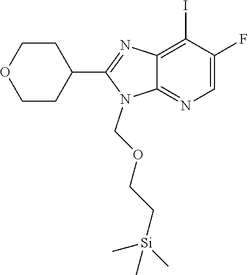 [SMILES: C[Si](C)(C)CCOCn1c(C2CCOCC2)nc2c(I)c(F)cnc21]

Under argon atmosphere at 5° C., to a solution of 6-fluoro-7-iodo-2-tetrahydropyran-4-yl-3H-imidazo[4,5-b]pyridine (400 mg, 1.15 mmol) in DMF (6 mL) was added portionwise sodium hydride (60% in oil, 65 mg, 1.62 mmol) and the reaction mixture was then stirred at 5° C. for 30 minutes, and 2-(trimethylsilyl)methoxymethyl chloride (270 μL, 1.5 mmol) was added dropwise. The whole mixture was stirred at 5° C. for 1.75 hours and slowly hydrolyzed with a saturated aqueous solution of NH₄Cl (10 mL). The whole mixture was warmed up to room temperature, and diluted with water (40 mL) and AcOEt (40 mL). The aqueous layer was extracted with AcOEt and the combined organic layers were dried over sodium sulfate, filtered and concentrated in vacuo. The resulting residue was purified on silica gel (SiO₂ 12 g) eluting with cyclohexane/AcOEt 100/0 to 80/20 to give 212 mg (38%) of 2-[(6-fluoro-7-iodo-2-tetrahydropyran-4-yl-imidazo[4,5-b]pyridin-3-yl)methoxy]ethyl-trimethyl-silane as a yellow solid.

### Step 6: tert-butyl 4-[6-fluoro-2-tetrahydropyran-4-yl-3-(2-trimethylsilylethoxymethyl)imidazo[4,5-b]pyridin-7-yl]piperidine-1-carboxylate

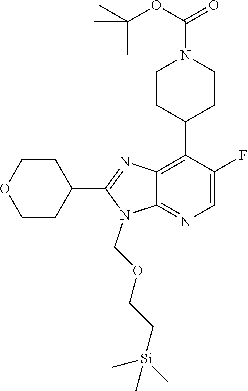 [SMILES: CC(C)(C)OC(=O)N1CCC(c2c(F)cnc3c2nc(C2CCOCC2)n3COCC[Si](C)(C)C)CC1]

In a 8 mL photochemical vial were charged nickel(II) chloride ethylene glycol dimethyl ether complex (11 mg, 0.05 mmol), 4,4′-di-tert-butyl-2,2′-bipyridine (13 mg, 0.05 mmol). Then, THF (1.5 mL) was added and the vial was sealed and stirred vigorously for 15 minutes. Then, potassium (1-(tert-butoxycarbonyl)piperidin-4-yl)trifluoroborate (160 mg, 0.55 mmol), and cesium carbonate (205 mg, 0.63 mmol) were added, followed by a degassed solution of 2-[(6-fluoro-7-iodo-2-tetrahydropyran-4-yl-imidazo[4,5-b]pyridin-3-yl)methoxy]ethyl-trimethyl-silane (200 mg, 0.42 mmol) in THF (4 mL) and (Ir[dF(CF₃)ppy]₂(dtbpy))PF₆ (10 mg, 0.009 mmol). The whole solution was bubbled with argon for 10 minutes and then submitted to irradiation with blue LEDs (450 nm, maintained at approximately room temperature by a desk fan) for 2 hours. After 2 hours, the reaction mixture was diluted with water (30 mL) and AcOEt (30 mL), and the aqueous layer was extracted with AcOEt. The combined organic layers were then dried over sodium sulfate, filtered and concentrated in vacuo. The resulting residue was purified on silica gel (SiO₂ 12 g) eluting with cyclohexane/AcOEt 8/2 to give 103 mg (76% yield) of tert-butyl 4-[6-fluoro-2-tetrahydropyran-4-yl-3-(2-trimethylsilylethoxymethyl)imidazo[4,5-b]pyridin-7-yl]piperidine-1-carboxylate as a yellow wax.

### Step 7: 6-fluoro-7-(4-piperidyl)-2-tetrahydropyran-4-yl-3H-imidazo[4,5-b]pyridine;hydrochloride

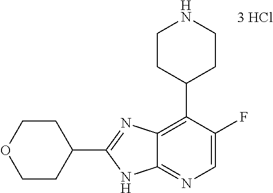 [SMILES: Fc1cnc2[nH]c(C3CCOCC3)nc2c1C1CCNCC1]

To a solution of tert-butyl 4-[6-fluoro-2-tetrahydropyran-4-yl-3-(2-trimethylsilylethoxymethyl)imidazo[4,5-b]pyridin-7-yl]piperidine-1-carboxylate (100 mg, 0.19 mmol) in MeOH (3 mL) was added a 4 M HCl solution (500 μL, 2 mmol) in dioxane and the whole mixture was stirred 12 hours at room temperature, then 4.5 hours at 40° C. 100 μL of a 4 M HCl solution in dioxane were added and the reaction mixture was stirred at room temperature for 2 hours and concentrated in vacuo. The resulting residue was triturated in DCM, and concentrated in vacuo to give 72 mg (93% yield) of 6-fluoro-7-(4-piperidyl)-2-tetrahydropyran-4-yl-3H-imidazo[4,5-b]pyridine;hydrochloride as a white solid which was used in the next step without further purification.

### Step 8: [2-amino-4-(pentafluoro-λ⁶-sulfanyl)phenyl]-[4-(6-fluoro-2-tetrahydropyran-4-yl-3H-imidazo[4,5-b]pyridin-7-yl)-1-piperidyl]methanone

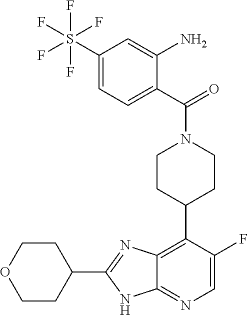 [SMILES: Nc1cc(S(F)(F)(F)(F)F)ccc1C(=O)N1CCC(c2c(F)cnc3[nH]c(C4CCOCC4)nc23)CC1]

Step 8 was performed following the protocol described in Example 131 using 2-amino-4-(pentafluoro-λ⁶-sulfanyl)benzoic acid to give 47 mg (50% yield) of [2-amino-4-(pentafluoro-λ⁶-sulfanyl)phenyl]-[4-(6-fluoro-2-tetrahydropyran-4-yl-3H-imidazo[4,5-b]pyridin-7-yl)-1-piperidyl]methanone as a white powder. LC/MS (m/z, M+H, Method 1): calc. 550.2, found 550.1; ¹H NMR (400 MHz, DMSO-d6, 100° C.) δ ppm 1.77-2.06 (m, 6H) 2.16-2.46 (m, 2H) 3.06-3.26 (m, 3H) 3.45-3.65 (m, 3H) 3.98 (dt, J=11.4, 3.3 Hz, 2H) 4.18 (br d, J=9.3 Hz, 2H) 5.48 (br s, 2H) 7.04 (dd, J=8.4, 2.2 Hz, 1H) 7.25 (d, J=8.4 Hz, 1H) 7.31 (d, J=2.2 Hz, 1H) 8.07-8.19 (m, 1H) 12.44-12.72 (m, 1H)

Examples 351-354: [2-amino-4-(pentafluoro-λ⁶-sulfanyl)phenyl]-[(cis)-2-methyl-4-(2-tetrahydropyran-4-yl-3H-imidazo[4,5-b]pyridin-7-yl)-1-piperidyl]methanone, Isomer 1, [2-amino-4-(pentafluoro-λ⁶-sulfanyl)phenyl]-[(trans)-2-methyl-4-(2-tetrahydropyran-4-yl-3H-imidazo[4,5-b]pyridin-7-yl)-1-piperidyl]methanone, Isomers 1 and 2, and [2-amino-4-(pentafluoro-k-sulfanyl)phenyl]-[(cis)-2-methyl-4-(2-tetrahydropyran-4-yl-3H-imidazo[4,5-b]pyridin-7-yl)-1-piperidyl]methanone, Isomer 2

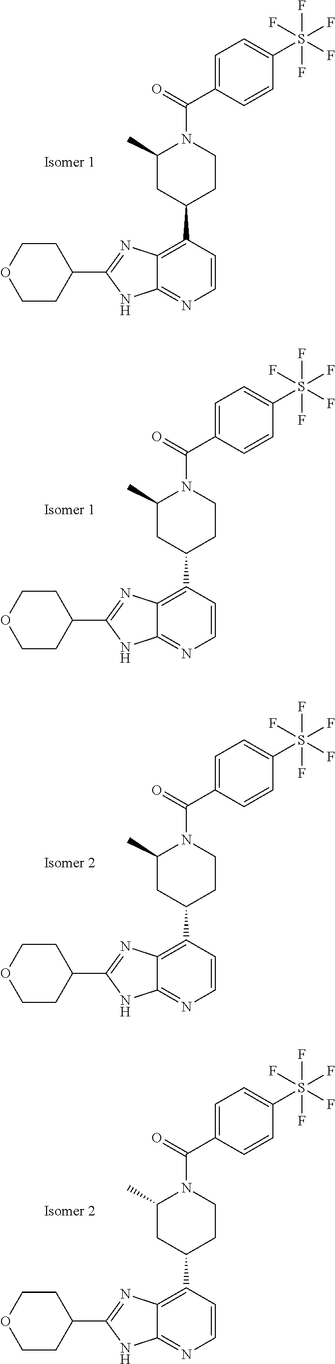 [SMILES: C[C@@H]1C[C@H](c2ccnc3[nH]c(C4CCOCC4)nc23)CCN1C(=O)c1ccc(S(F)(F)(F)(F)F)cc1 ; C[C@@H]1C[C@@H](c2ccnc3[nH]c(C4CCOCC4)nc23)CCN1C(=O)c1ccc(S(F)(F)(F)(F)F)cc1 ; C[C@@H]1C[C@@H](c2ccnc3[nH]c(C4CCOCC4)nc23)CCN1C(=O)c1ccc(S(F)(F)(F)(F)F)cc1 ; C[C@H]1C[C@@H](c2ccnc3[nH]c(C4CCOCC4)nc23)CCN1C(=O)c1ccc(S(F)(F)(F)(F)F)cc1]

Examples 351-354 were prepared by a procedure similar to that of step 5 of Examples 339-342 using 2-amino-4-(pentafluoro-λ⁶-sulfanyl)benzoic acid, followed by chiral separation using Method CS8 (Whelk 01 SS column (5 μm, 350×76.5 mm), (flow rate 250 mL/min)), followed by further chiral separation using Method CS18.

### Examples 355 and 356: (cis)-[2-Amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone and (trans)-[2-Amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone

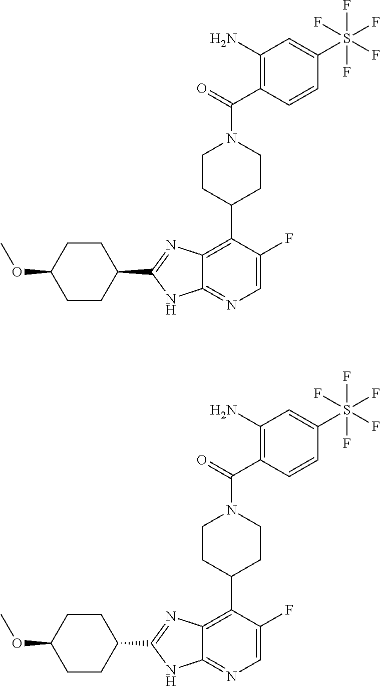 [SMILES: CO[C@H]1CC[C@@H](c2nc3c(C4CCN(C(=O)c5ccc(S(F)(F)(F)(F)F)cc5N)CC4)c(F)cnc3[nH]2)CC1 ; CO[C@H]1CC[C@H](c2nc3c(C4CCN(C(=O)c5ccc(S(F)(F)(F)(F)F)cc5N)CC4)c(F)cnc3[nH]2)CC1]

### Step 1: 2-Chloro-5-fluoro-3-nitro-pyridin-4-amine

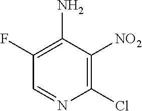 [SMILES: Nc1c(F)cnc(Cl)c1[N+](=O)[O-]]

To a solution of 2-chloro-5-fluoro-pyridin-4-amine (25.0 g, 171 mmol) in H₂SO₄ (300 mL) was added potassium nitrate (34.5 g, 341 mmol) slowly at −5° C. The mixture was stirred at room temperature for 16 hours. The mixture was then poured into iced water, the solid filtered, collected and dried in vacuo. The solid was dissolved in H₂SO₄ (200 mL) and the solution stirred at room temperature for 16 hours. The reaction was poured into iced water (600 mL), adjust to pH=7 with NH₃—H₂O and exacted with EA (3×600 mL). The combined organic layers were washed with brine (600 mL), dried over Na₂SO₄, filtered and evaporated under reduced pressure to give 27 g (83% yield) of 2-chloro-5-fluoro-3-nitro-pyridin-4-amine. LCMS: ESI m/z (M+H) 191.9

### Step 2: 5-Fluoro-N-2-[(4-methoxyphenyl)methyl]-3-nitro-pyridine-2,4-diamine

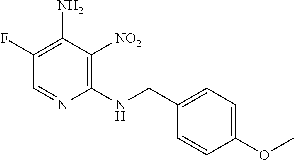 [SMILES: COc1ccc(CNc2ncc(F)c(N)c2[N+](=O)[O-])cc1]

To s solution of 2-chloro-5-fluoro-3-nitro-pyridin-4-amine (27 g, 141 mmol) in DMSO (200 mL) were added N,N-diethylethanamine (43 g, 424 mmol) and 4-methoxybenzylamine (41.4 g, 282 mmol) and the reaction stirred at 150° C. for 5 hours. The cooled mixture was quenched with water (200 mL) and extracted with EtOAc (3×100 mL). The combined organic layers were washed with brine (100 mL), dried over Na₂SO₄, filtered and concentrated in vacuo. The crude product was purified by flash chromatography to afford 35 g (85% yield) of 5-fluoro-N-2-[(4-methoxyphenyl)methyl]-3-nitro-pyridine-2,4-diamine. LCMS: ESI m/z (M+H) 292.9

### Step 3: 4-Chloro-5-fluoro-N-[(4-methoxyphenyl)methyl]-3-nitro-pyridin-2-amine

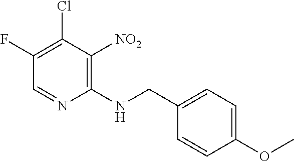 [SMILES: COc1ccc(CNc2ncc(F)c(Cl)c2[N+](=O)[O-])cc1]

To a solution of 5-fluoro-N-2-[(4-methoxyphenyl)methyl]-3-nitro-pyridine-2,4-diamine (1 g, 3.4 mmol) in MeCN (15 mL) were added tert-butyl nitrite (529 mg, 5.1 mmol) and CuCl (508 mg, 5.1 mmol) and the reaction stirred at 70° C. for 3 hours. The cooled mixture was quenched with water (30 mL) and extracted with EtOAc (3×20 mL). The combined organic layers were washed with brine (20 mL), dried over Na₂SO₄, filtered and concentrated in vacuo. The crude was purified by flash chromatography to afford to 500 mg (47% yield) of 4-chloro-5-fluoro-N-[(4-methoxyphenyl)methyl]-3-nitro-pyridin-2-amine. LCMS: ESI (M+H), m/z 311.8

### Step 4: tert-Butyl 4-[5-fluoro-2-[(4-methoxyphenyl)methylamino]-3-nitro-4-pyridyl]-3,6-dihydro-2H-pyridine-1-carboxylate

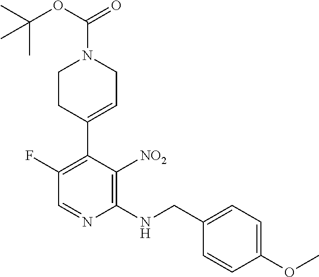 [SMILES: COc1ccc(CNc2ncc(F)c(C3=CCN(C(=O)OC(C)(C)C)CC3)c2[N+](=O)[O-])cc1]

A mixture of 4-chloro-5-fluoro-N-[(4-methoxyphenyl)methyl]-3-nitro-pyridin-2-amine (500 mg, 1.60 mmol), tert-butyl 4-(4,4,5,5-tetramethyl-1,3,2-dioxaborolan-2-yl)-3,6-dihydro-2H-pyridine-1-carboxylate (496 g, 1.60 mmol), [1,1′-bis(diphenylphosphino)ferrocene]dichloropalladium(II) (59 mg, 0.08 mmol), and disodium carbonate (510 mg, 4.8 mmol) in 1,4-dioxane (8 mL) and water (2 mL) was stirred at 80° C. for 3 hours under N₂ atmosphere. The mixture was then diluted with water (30 mL), and extracted with ethyl acetate (2×20 mL). The organic layers were combined and washed with brine (20 mL), then it was dried over Na₂SO₄. The mixture was concentrated and the residue was purified by silica gel flash chromatography to give 500 mg (68% yield) of tert-butyl 4-[5-fluoro-2-[(4-methoxyphenyl)methylamino]-3-nitro-4-pyridyl]-3,6-dihydro-2H-pyridine-1-carboxylate. LCMS: ESI m/z (M+H) 458.8

### Step 5: tert-Butyl 4-(2,3-diamino-5-fluoro-4-pyridyl)piperidine-1-carboxylate

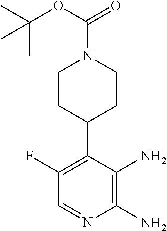 [SMILES: CC(C)(C)OC(=O)N1CCC(c2c(F)cnc(N)c2N)CC1]

To a solution of tert-butyl 4-[5-fluoro-2-[(4-methoxyphenyl)methylamino]-3-nitro-4-pyridyl]-3,6-dihydro-2H-pyridine-1-carboxylate (4.2 g, 9.16 mmol) in ethanol (100 mL) was added palladium (1.95 g, 18.3 mmol). The mixture was stirred at 50° C. for 7 days under 1 atmosphere of H₂. The mixture was then filtered and concentrated and purified by flash chromatography to give 1.91 g (67% yield) of tert-butyl 4-(2,3-diamino-5-fluoro-4-pyridyl)piperidine-1-carboxylate. LCMS: ESI (M+H) m/z 311.3

### Step 6: tert-Butyl 4-[6-fluoro-2-(4-methoxycyclohexyl)-3H-imidazo[4,5-b]pyridin-7-yl]piperidine-1-carboxylate

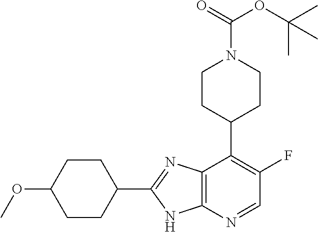 [SMILES: COC1CCC(c2nc3c(C4CCN(C(=O)OC(C)(C)C)CC4)c(F)cnc3[nH]2)CC1]

In a microwave vial, were successively introduced tert-butyl 4-(2,3-diamino-5-fluoro-4-pyridyl)piperidine-1-carboxylate (85 mg, 0.25 mmol), 4-methoxycyclohexanecarbaldehyde (37 mg, 0.28 mmol) and DMF (0.5 mL). To this solution, was then added sodium metabisulfite (68 mg, 0.36 mmol). The vial was then sealed and submitted to microwave at 125° C. for 90 minutes. After 90 minutes, the vial was cooled down to room temperature, opened, diluted with AcOEt, transferred to a separating funnel, and extracted twice with AcOEt. The combined organic layers were then washed with water, dried over magnesium sulfate, filtered, concentrated in vacuo. The resulting residue was purified by flash chromatography to give 80 mg (57% yield) of tert-butyl 4-[6-fluoro-2-(4-methoxycyclohexyl)-3H-imidazo[4,5-b]pyridin-7-yl]piperidine-1-carboxylate. LC/MS (m/z, M+H, Method 1): calc. 433.5, found 433.2

### Step 7: 6-Fluoro-2-(4-methoxycyclohexyl)-7-(4-piperidyl)-3H-imidazo[4,5-b]pyridine;hydrochloride

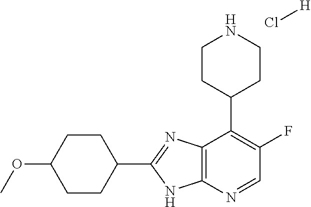

To a solution of tert-butyl 4-[6-fluoro-2-(4-methoxycyclohexyl)-3H-imidazo[4,5-b]pyridin-7-yl]piperidine-1-carboxylate (80 mg, 0.18 mmol) in methanol (2 mL), was added 0.48 mL of a 5 M HCl solution in iPrOH. The resulting mixture was then stirred at room temperature for 12 hours. After 12 hours, the reaction mixture was concentrated in vacuo, and the resulting residue was triturated in diethyl ether, filtered, washed with diethyl ether to give 50 mg (63% yield) of 6-fluoro-2-(4-methoxycyclohexyl)-7-(4-piperidyl)-3H-imidazo[4,5-b]pyridine;hydrochloride as a yellow solid. LC/MS (m/z, M+H-HCl, Method 1): calc. 333.5, found 333.2

### Step 8: (cis)-[2-Amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone and (trans)-[2-Amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone

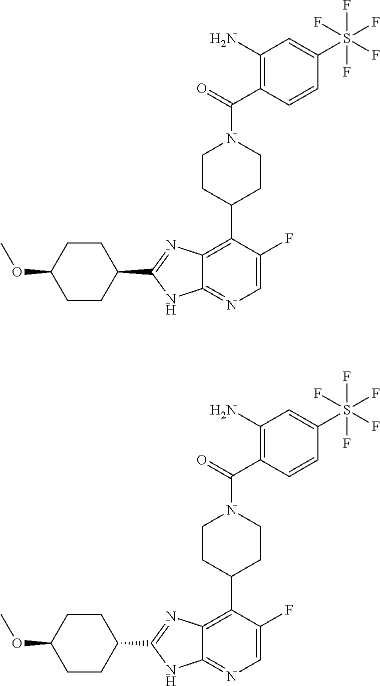 [SMILES: CO[C@H]1CC[C@@H](c2nc3c(C4CCN(C(=O)c5ccc(S(F)(F)(F)(F)F)cc5N)CC4)c(F)cnc3[nH]2)CC1 ; CO[C@H]1CC[C@H](c2nc3c(C4CCN(C(=O)c5ccc(S(F)(F)(F)(F)F)cc5N)CC4)c(F)cnc3[nH]2)CC1]

To a solution of 6-fluoro-2-(4-methoxycyclohexyl)-7-(4-piperidyl)-3H-imidazo[4,5-b]pyridine;hydrochloride (50 mg, 0.12 mmol) in DMF (2 mL) was added N,N-diisopropylethylamine (64 mg, 0.49 mmol). The resulting mixture was cooled down to −20° C., and TATU (42 mg, 0.13 mmol) and 2-amino-4-(pentafluoro-λ⁶-sulfanyl)benzoic acid (34 mg, 0.13 mmol) were added. The reaction mixture was stirred for one hour at this temperature, then diluted with water, transferred to a separating funnel, extracted twice with AcOEt. The combined organic layers were then dried over magnesium sulfate, filtered, concentrated in vacuo. The resulting residue was triturated in diethyl ether and filtered to give 66 mg of the cis/trans mixture as a white solid, which was then purified by flash chromatography to give 14 mg (20% yield) of (cis)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone and 22 mg (31% yield) of (trans)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone as yellow solids.

Example 355 (cis): LC/MS (m/z, M+H, Method 1): calc. 578.6, found 578.1

Example 356 (trans): LC/MS (m/z, M+H, Method 1): calc. 578.6, found 578.1; ¹H NMR (400 MHz, DMSO-d6, 100° C.) δ ppm 1.24-1.37 (m, 2H), 1.63-1.75 (m, 2H), 1.80 (br d, J=12.3 Hz, 2H), 2.11 (br d, J=10.5 Hz, 4H), 2.23-2.38 (m, 2H), 2.79-2.89 (m, 1H), 3.09 (br t, J=12.5 Hz, 2H), 3.18-3.24 (m, 1H), 3.28 (s, 3H), 3.47-3.58 (m, 1H), 4.08-4.23 (m, 2H), 5.46 (s, 2H), 7.01 (br d, J=8.3 Hz, 1H), 7.23 (br d, J=8.3 Hz, 1H), 7.29 (s, 1H), 8.10 (br s, 1H), 12.29-12.57 (m, 1H)

### Example 357: [4-(6-fluoro-2-tetrahydropyran-4-yl-3H-imidazo[4,5-b]pyridin-7-yl)-1-piperidyl]-[4-(pentafluoro-λ⁶-sulfanyl)phenyl]methanone

Example 357 was prepared following a procedure similar to that of Example 131 using 4-(pentafluoro-λ⁶-sulfanyl)benzoic acid and 6-fluoro-7-(4-piperidyl)-2-tetrahydropyran-4-yl-3H-imidazo[4,5-b]pyridine;hydrochloride (described in step 7 of Example 78). LC/MS (m/z, M+H): 535; ¹H NMR (400 MHz, DMSO-d6, 100° C.) δ ppm 1.79-1.88 (m, 2H), 1.88-1.99 (m, 4H), 2.22-2.39 (m, 2H), 3.08-3.23 (m, 3H), 3.45-3.61 (m, 3H), 3.92-4.00 (m, 2H), 4.01-4.32 (m, 2H), 7.64 (d, J=9 Hz, 2H), 7.85-8.03 (m, 2H), 8.12 (d, J=3 Hz, 1H), 12.31-12.81 (m, 1H)

### Examples 358 and 359: [2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone, Isomers 1 and 2

Examples 358 and 359 were obtained by chiral separation of the compound of Example 295 using Method CS1 (chiralpak IC column (20 μm, 76.5×400 mm), eluting with (n-Heptane 80% EtOH 20%)+0.1% TEA).

### Examples 360 and 361: [2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone, Isomers 1 and 2

Examples 360 and 361 were obtained by chiral separation of the compound of Example 347 using Method CS17.

### Examples 362 and 363: [4-(pentafluoro-λ⁶-sulfanyl)phenyl]-[3-(2-tetrahydropyran-4-yl-3H-imidazo[4,5-b]pyridin-7-yl)pyrrolidin-1-yl]methanone, Isomers 1 and 2

Examples 362 and 363 were obtained by chiral separation of the compound of Example 348 using Method CS12.

### Example 364: (rac)-[4-(6-fluoro-2-morpholin-2-yl-3H-imidazo[4,5-b]pyridin-7-yl)-1-piperidyl]-[4-(pentafluoro-λ⁶-sulfanyl)phenyl]methanone

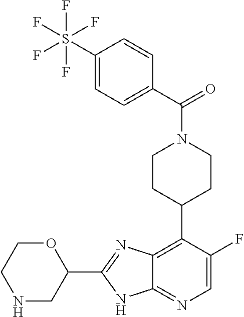 [SMILES: O=C(c1ccc(S(F)(F)(F)(F)F)cc1)N1CCC(c2c(F)cnc3[nH]c(C4CNCCO4)nc23)CC1]

### Step 1: [4-(2,3-diamino-5-fluoro-4-pyridyl)-1-piperidyl]-[4-(pentafluoro-λ⁶-sulfanyl)phenyl]methanone

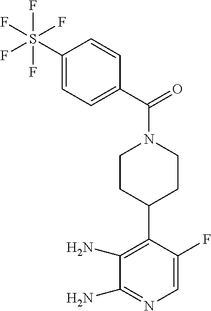 [SMILES: Nc1ncc(F)c(C2CCN(C(=O)c3ccc(S(F)(F)(F)(F)F)cc3)CC2)c1N]

Step 1 was performed following the protocol described for the preparation of Example 131 using 5-fluoro-4-(4-piperidyl)pyridine-2,3-diamine;hydrochloride (described in step 1 of Example 429) and 4-(pentafluoro-λ⁶-sulfanyl)benzoic acid to give 167 mg (87% yield) of [4-(2,3-diamino-5-fluoro-4-pyridyl)-1-piperidyl]-[4-(pentafluoro-λ⁶-sulfanyl)phenyl]methanone as a pink solid.

### Step 2: (rac)-[4-(6-fluoro-2-morpholin-2-yl-3H-imidazo[4,5-b]pyridin-7-yl)-1-piperidyl]-[4-(pentafluoro-λ⁶-sulfanyl)phenyl]methanone

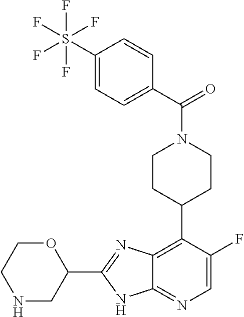 [SMILES: O=C(c1ccc(S(F)(F)(F)(F)F)cc1)N1CCC(c2c(F)cnc3[nH]c(C4CNCCO4)nc23)CC1]

To a solution of [4-(2,3-diamino-5-fluoro-4-pyridyl)-1-piperidyl]-[4-(pentafluoro-λ⁶-sulfanyl)phenyl]methanone (125 mg, 0.28 mmol) in DCM (7 mL) was added at 5° C., 4-tert-butoxycarbonylmorpholine-2-carboxylic acid (70 mg, 0.30 mmol), triethylamine (100 μL, 0.72 mmol) and 2-chloro-1-methylpyridinium iodide (80 mg, 0.31 mmol). The reaction mixture was stirred at 5° C. for 30 minutes and then at room temperature for 3 hours. After 3 hours, the reaction mixture was concentrated in vacuo and the resulting residue was solubilized in MeOH and filtered through an SCX cartridge eluting with MeOH first, followed by a 2M solution of NH₃ in MeOH. The filtrate was concentrated in vacuo and the resulting residue was purified on silica gel (SiO₂ 12 g) eluting with DCM/MeOH 95/5. The desired collected fractions were concentrated in vacuo to give a colorless wax, which eluted through an SCK cartridge (2.5 g) with MeOH followed by a 2M solution of NH₃ in MeOH to give 34 mg (22% yield) of (rac)-[4-(6-fluoro-2-morpholin-2-yl-3H-imidazo[4,5-b]pyridin-7-yl)-1-piperidyl]-[4-(pentafluoro-λ⁶-sulfanyl)phenyl]methanone as a white powder. LC/MS (m/z, M+H, Method 1): calc. 536.1, found 536.0; ¹H NMR (400 MHz, DMSO-d6, 100° C.) 6 ppm 1.82 (br d, J=11.4 Hz, 2H), 2.16-2.33 (m, 2H), 2.79-2.84 (m, 2H), 2.96-3.20 (partially hidden m, 6H), 3.52-3.72 (m, 2H), 3.88 (dt, J=11.1, 2×2.8 Hz, 1H), 3.97-4.41 (m, 2H), 4.69 (dd, J=9.5, 2.9 Hz, 1H), 7.65 (br d, J=8.5 Hz, 2H), 7.92-7.99 (m, 2H, 2H), 8.17 (d, J=3.4 Hz, 1H)

### Example 365: [4-[6-fluoro-2-(2-oxabicyclo[2.2.2]octan-4-yl)-3H-imidazo[4,5-b]pyridin-7-yl]-1-piperidyl]-[4-(pentafluoro-λ⁶-sulfanyl)phenyl]methanone

Example 365 was prepared following a procedure similar to that of Examples 355 and 356 using 2-oxabicyclo[2.2.2]octane-4-carbaldehyde in step 6, and 4-(pentafluoro-λ⁶-sulfanyl)benzoic acid in step 8 (with TBTU instead of TATU). LC/MS (m/z, M+H): 561; ¹H NMR (400 MHz, DMSO-d6, 100° C.) δ ppm 1.69-1.85 (m, 4H), 2.04-2.18 (m, 6H), 2.24-2.33 (m, 2H), 3.12 (br t, J=13.6 Hz, 2H), 3.57 (tt, J=12.3 Hz, J=3.8 Hz, 1H), 3.79-3.84 (m, 1H), 4.05 (br s, 2H), 4.08-4.32 (m, 2H), 7.64 (br d, J=8.8 Hz, 2H), 7.96 (d, J=8.8 Hz, 2H), 8.13 (d, J=3.3 Hz, 1H), 12.39-12.75 (m, 1H)

### Example 366: [2-amino-4-(pentafluoro-λ⁶-sulfanyl)phenyl]-[4-[6-fluoro-2-(2-oxabicyclo[2.2.2]octan-4-yl)-3H-imidazo[4,5-b]pyridin-7-yl]-1-piperidyl]methanone

Example 366 was prepared following a procedure similar to that of Examples 355 and 356 using 2-oxabicyclo[2.2.2]octane-4-carbaldehyde in step 6, and with TBTU instead of TATU in step 8. LC/MS (m/z, M+H): 576; ¹H NMR (400 MHz, DMSO-d6, 100° C.) δ ppm 1.68-1.83 (m, 4H), 2.00-2.17 (m, 6H), 2.32-2.40 (m, 2H), 3.03-3.16 (m, 2H), 3.49-3.59 (m, 1H), 3.80-3.83 (m, 1H), 4.04 (s, 2H), 4.07-4.23 (m, 2H), 5.44 (br s, 2H), 7.01 (dd, J=8.5, 2.3 Hz, 1H), 7.22 (d, J=8.5 Hz, 1H), 7.29 (d, J=2.3 Hz, 1H), 8.11 (br d, J=2.5 Hz, 1H), 12.55-12.64 (m, 1H)

### Example 367: (rac)-[4-(6-fluoro-2-tetrahydropyran-3-yl-3H-imidazo[4,5-b]pyridin-7-yl)-1-piperidyl]-[4-(pentafluoro-λ⁶-sulfanyl)phenyl]methanone

Example 367 was prepared following a procedure similar to that of Examples 355 and 356 using tetrahydropyran-3-carbaldehyde in step 6, and 4-(pentafluoro-λ⁶-sulfanyl)benzoic acid in step 8 (with TBTU instead of TATU).

### Example 368: (cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone

Example 368 was prepared following a procedure similar to that of Example 293 using (cis)-Intermediate I (prepared following a procedure similar to Intermediate I from (cis)-3-hydroxycyclobutanecarboxylic acid) in step 1, 5-6N HCl in iPrOH in step 2, and using 4-(pentafluoro-λ⁶-sulfanyl)benzoic acid in step 3.

### Example 369: (cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone

Example 369 was prepared following a procedure similar to that of Example 293 using (cis)-Intermediate I (prepared following a procedure similar to Intermediate I from (cis)-3-hydroxycyclobutanecarboxylic acid) in step 1, 5-6N HCl in iPrOH in step 2, and using 2-amino-4-(pentafluoro-λ⁶-sulfanyl)benzoic acid in step 3.

### Example 370: [2-fluoro-4-(pentafluoro-λ⁶-sulfanyl)phenyl]-[4-(2-tetrahydropyran-4-yl-3H-imidazo[4,5-b]pyridin-7-yl)-1-piperidyl]methanone

Example 370 was prepared following a procedure similar to that of step 5 of Examples 1 and 2 using 2-fluoro-4-(pentafluorosulfur)benzoic acid and 7-(4-piperidyl)-2-tetrahydropyran-4-yl-3H-imidazo[4,5-b]pyridine;dihydrochloride.

### Example 371: (rac)-[2-amino-4-(pentafluoro-λ⁶-sulfanyl)phenyl]-[4-(6-fluoro-2-tetrahydropyran-3-yl-3H-imidazo[4,5-b]pyridin-7-yl)-1-piperidyl]methanone

Example 371 was prepared following a procedure similar to that of Examples 355 and 356 using tetrahydropyran-3-carbaldehyde in step 6, and with TBTU instead of TATU in step 8.

### Examples 372 and 373: (trans)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone and (cis)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone

Examples 372 and 373 were prepared following a procedure similar to that of Examples 355 and 356 using 4-(pentafluoro-λ⁶-sulfanyl)benzoic acid in step 8.

Example 372 (cis-isomer): LC/MS (m/z, M+H): 563; ¹H NMR (400 MHz, DMSO-d6, 100° C.) δ ppm 1.24-1.37 (m, 2H), 1.64-1.75 (m, 2H), 1.81 (br d, J=11.3 Hz, 2H), 2.08-2.15 (m, 4H), 2.22-2.36 (m, 2H), 2.85 (tt, J=11.5, 3.3 Hz, 1H), 3.12 (br t, J=12.0 Hz, 2H), 3.18-3.25 (m, 1H), 3.28 (s, 3H), 3.49-3.61 (m, 1H), 4.17 (m, 2H), 7.64 (br d, J=8.5 Hz, 2H), 7.95 (d, J=8.5 Hz, 2H), 8.10 (d, J=3.3 Hz, 1H), 12.38-12.53 (m, 1H)

### Example 374: (rac)-[4-(6-fluoro-2-tetrahydrofuran-3-yl-3H-imidazo[4,5-b]pyridin-7-yl)-1-piperidyl]-[4-(pentafluoro-λ⁶-sulfanyl)phenyl]methanone

Example 374 was prepared following a procedure similar to that of Examples 355 and 356 using tetrahydrofuran-3-carbaldehyde in step 6 and 4-(pentafluoro-λ⁶-sulfanyl)benzoic acid and triethylamine instead of DIPEA in step 8. LC/MS (m/z, M+H): 521; ¹H NMR (400 MHz, DMSO-d6, 100° C.) δ ppm 1.84 (br d, J=12.5 Hz, 2H), 2.27-2.40 (m, 3H), 3.07-3.21 (m, 2H), 3.51-3.65 (m, 1H), 3.65-3.76 (m, 1H), 3.82-3.91 (m, 1H), 3.91-4.04 (m, 2H), 4.12 (t, J=8.0 Hz, 1H), 4.07-4.29 (m, 1H), 7.66 (br d, J=8.5 Hz, 2H), 7.92-7.99 (m, 2H), 8.15 (br d, J=3.0 Hz, 1H), 12.32-12.81 (m, 1H)

### Example 375: (rac)-[2-amino-4-(pentafluoro-λ⁶-sulfanyl)phenyl]-[4-[6-fluoro-2-(oxepan-4-yl)-3H-imidazo[4,5-b]pyridin-7-yl]-1-piperidyl]methanone

Example 375 was prepared following a procedure similar to that of Examples 355 and 356 using oxepane-4-carbaldehyde in step 6, and the procedure of Example 131 using 2-amino-4-(pentafluoro-λ⁶-sulfanyl)benzoic acid in place of step 8.

### Example 376: (rac)-[4-[6-fluoro-2-(oxepan-4-yl)-3H-imidazo[4,5-b]pyridin-7-yl]-1-piperidyl]-[4-(pentafluoro-λ⁶-sulfanyl)phenyl]methanone

Example 376 was prepared following a procedure similar to that of Examples 355 and 356 using oxepane-4-carbaldehyde in step 6, and the procedure of Example 131 using 4-(pentafluoro-λ⁶-sulfanyl)benzoic acid in place of step 8.

### Example 377: (rac)-[2-amino-4-(pentafluoro-λ⁶-sulfanyl)phenyl]-[4-(6-fluoro-2-tetrahydrofuran-3-yl-3H-imidazo[4,5-b]pyridin-7-yl)-1-piperidyl]methanone

Example 377 was prepared following a procedure similar to that of Examples 355 and 356 using tetrahydrofuran-3-carbaldehyde in step 6, and 2-amino-4-(pentafluoro-λ⁶-sulfanyl)benzoic acid and triethylamine instead of DIPEA in step 8.

### Example 378: [4-[2-[(2R,6R)-6-methylmorpholin-2-yl]-3H-imidazo[4,5-b]pyridin-7-yl]-1-piperidyl]-[4-(pentafluoro-λ⁶-sulfanyl)phenyl]methanone;dihydrochloride

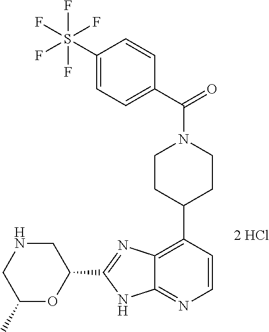 [SMILES: C[C@@H]1CNC[C@H](c2nc3c(C4CCN(C(=O)c5ccc(S(F)(F)(F)(F)F)cc5)CC4)ccnc3[nH]2)O1]

### Step 1: tert-butyl (2R,6R)-2-[[3-amino-4-[1-[4-(pentafluoro-λ⁶-sulfanyl)benzoyl]-4-piperidyl]-2-pyridyl]carbamoyl]-6-methyl-morpholine-4-carboxylate

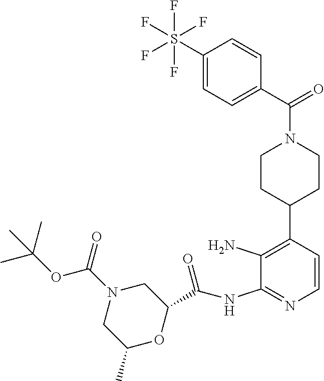 [SMILES: C[C@@H]1CN(C(=O)OC(C)(C)C)C[C@H](C(=O)Nc2nccc(C3CCN(C(=O)c4ccc(S(F)(F)(F)(F)F)cc4)CC3)c2N)O1]

Step 1 was performed following the protocol described in step 5 of Examples 1 and 2 using [4-(2,3-diamino-4-pyridyl)-1-piperidyl]-[4-(pentafluoro-λ⁶-sulfanyl)phenyl]methanone (described in step 1 of Example 364) and (2R,6R)-4-(tert-butoxycarbonyl)-6-methylmorpholine-2-carboxylic acid to give 75 mg (100% yield) of crude tert-butyl (2R,6R)-2-[[3-amino-4-[1-[4-(pentafluoro-λ⁶-sulfanyl)benzoyl]-4-piperidyl]-2-pyridyl]carbamoyl]-6-methyl-morpholine-4-carboxylate which was used in the next step without further purification.

### Step 2: tert-butyl (2R,6R)-2-methyl-6-[7-[1-[4-(pentafluoro-λ⁶-sulfanyl)benzoyl]-4-piperidyl]-3H-imidazo[4,5-b]pyridin-2-yl]morpholine-4-carboxylate

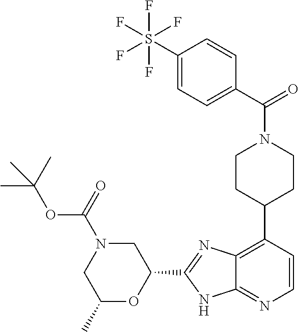 [SMILES: C[C@@H]1CN(C(=O)OC(C)(C)C)C[C@H](c2nc3c(C4CCN(C(=O)c5ccc(S(F)(F)(F)(F)F)cc5)CC4)ccnc3[nH]2)O1]

To a solution of tert-butyl (2R,6R)-2-[[3-amino-4-[1-[4-(pentafluoro-λ⁶-sulfanyl)benzoyl]-4-piperidyl]-2-pyridyl]carbamoyl]-6-methyl-morpholine-4-carboxylate (75 mg, 0.12 mmol) in DMF (1 mL) was added cesium fluoride (51 mg, 0.34 mmol), and the resulting reaction mixture was stirred at 110° C. for 12 hours; After 12 hours, the reaction mixture was cooled to room temperature, diluted with water and AcOEt, transferred to a separating funnel, and extracted twice with AcOEt. The combined organic layers were washed with water, dried over magnesium sulfate, filtered and concentrated in vacuo. The resulting residue was purified on silica gel (SiO₂ 12 g) with AcOEt, to give 26 mg (34% yield over two steps) of tert-butyl (2R,6R)-2-methyl-6-[7-[1-[4-(pentafluoro-λ⁶-sulfanyl)benzoyl]-4-piperidyl]-3H-imidazo[4,5-b]pyridin-2-yl]morpholine-4-carboxylate as a white solid.

### Step 3: [4-[2-[(2R,6R)-6-methylmorpholin-2-yl]-3H-imidazo[4,5-b]pyridin-7-yl]-1-piperidyl]-[4-(pentafluoro-λ⁶-sulfanyl)phenyl]methanone;dihydrochloride

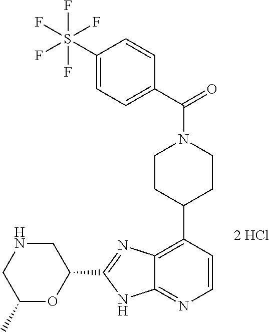 [SMILES: C[C@@H]1CNC[C@H](c2nc3c(C4CCN(C(=O)c5ccc(S(F)(F)(F)(F)F)cc5)CC4)ccnc3[nH]2)O1]

To (2R,6R)-2-methyl-6-[7-[1-[4-(pentafluoro-λ⁶-sulfanyl)benzoyl]-4-piperidyl]-3H-imidazo[4,5-b]pyridin-2-yl]morpholine-4-carboxylate (25 mg, 0.04 mmol) was added 2 mL of a 5-6 M HCl solution in i-PrOH. The whole mixture was stirred at room temperature for 40 minutes, then concentrated in vacuo to give 22 mg (92% yield) of [4-[2-[(2R,6R)-6-methylmorpholin-2-yl]-3H-imidazo[4,5-b]pyridin-7-yl]-1-piperidyl]-[4-(pentafluoro-λ⁶-sulfanyl)phenyl]methanone;dihydrochloride as a white solid. LC/MS (m/z, M+H-2HCl, Method 1): calc. 532.2, found 532.1; ¹H NMR (400 MHz, DMSO-d6, 100° C.) δ ppm 1.28 (d, J=6.3 Hz, 3H), 1.81-2.03 (m, 4H), 2.78-3.70 (partially hidden m, 9H), 4.11-4.30 (m, 2H), 5.25 (dd, J=11.3, 2.6 Hz, 1H), 7.19 (d, J=5.0 Hz, 1H), 7.69 (d, J=8.3 Hz, 2H), 7.95-8.00 (m, 2H), 8.32 (d, J=5.0 Hz, 1H), 9.52-10.01 (m, 2H)

### Example 379: [4-[6-fluoro-2-(1-methoxy-1-methyl-ethyl)-3H-imidazo[4,5-b]pyridin-7-yl]-1-piperidyl]-[4-(pentafluoro-λ⁶-sulfanyl)phenyl]methanone

Example 379 was prepared following a procedure similar to that of Examples 355 and 356 using 2-methoxy-2-methyl-propanal in step 6, and 4-(pentafluoro-λ⁶-sulfanyl)benzoic acid in step 8. LC/MS (m/z, M+H): 523; ¹H NMR (400 MHz, DMSO-d6, 100° C.) δ ppm 1.63 (s, 6H), 1.83 (br d, J=12.3 Hz, 2H), 2.19-2.42 (m, 2H), 3.06-3.20 (m, 2H), 3.11 (s, 3H), 3.61 (tt, J=12.3, 3.5 Hz, 1H), 3.9-4.5 (m, 2H), 7.64 (br d, J=8.5 Hz, 2H), 7.95 (m, 2H), 8.17 (d, J=3.3 Hz, 1H), 12.57-12.76 (m, 1H)

### Example 380: [2-amino-4-(pentafluoro-λ⁶-sulfanyl)phenyl]-[4-[2-[4-hydroxy-4-(trifluoromethyl)cyclohexyl]-3H-imidazo[4,5-b]pyridin-7-yl]-1-piperidyl]methanone

Example 380 was prepared following a procedure similar to that of step 1 of Example 293 using 4-hydroxy-4-(trifluoromethyl)cyclohexanecarboxylic acid, followed by a procedure similar to that of step 4 of Examples 1 and 2, followed by a procedure similar to that of step 3 of Example 293 using 2-amino-4-(pentafluoro-λ⁶-sulfanyl)benzoic acid.

### Example 381: [2-amino-4-(pentafluoro-λ⁶-sulfanyl)phenyl]-[4-[6-fluoro-2-(3-methoxy-1-bicyclo[1.1.1]pentanyl)-3H-imidazo[4,5-b]pyridin-7-yl]-1-piperidyl]methanone

Example 381 was prepared following a procedure similar to that of step 6 of Examples 355 and 356 using 3-methoxybicyclo[1.1.1]pentane-1-carbaldehyde, followed by a procedure similar to that of step 7 of Examples 355 and 356, followed by a procedure similar to that of Example 131 using 2-amino-4-(pentafluoro-λ⁶-sulfanyl)benzoic acid. LC/MS (m/z, M+H): 562; ¹H NMR (400 MHz, DMSO-d6, 100° C.) δ ppm 1.80 (br d, J=11.6 Hz, 2H), 2.22-2.29 (m, 2H), 2.31 (s, 6H), 2.93 (br s, 3H), 3.10 (br t, J=12.6 Hz, 2H), 3.50-3.63 (m, 1H), 4.04-4.22 (m, 2H), 5.44 (br s, 2H), 7.02 (dd, J=8.3, 2 Hz, 1H), 7.22 (d, J=8.3 Hz, 1H), 7.29 (d, J=2.1 Hz, 1H), 8.13 (br s, 1H), 12.77 (m, 1H)

### Example 382: [4-[6-fluoro-2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone

Example 381 was prepared following a procedure similar to that of step 6 of Examples 355 and 356 using 3-methoxybicyclo[1.1.1]pentane-1-carbaldehyde, followed by a procedure similar to that of step 7 of Examples 355 and 356, followed by a procedure similar to that of Example 131 using 4-(pentafluoro-λ⁶-sulfanyl)benzoic acid. LC/MS (m/z, M+H): 547; ¹H NMR (400 MHz, DMSO-d6, 100° C.) δ ppm 1.81 (br d, J=12.5 Hz, 2H), 2.17-2.29 (m, 2H), 2.32 (br s, 6H), 3.07-3.18 (m, 2H), 3.29 (s, 3H), 3.51-3.67 (m, 1H), 3.91-4.35 (m, 2H), 7.64 (br d, J=8.0 Hz, 2H), 7.96 (br d, J=8.0 Hz, 2H), 8.12-8.15 (m, 1H), 12.73-12.93 (m, 1H)

### Example 383: (rac)-[2-amino-4-(pentafluoro-λ⁶-sulfanyl)phenyl]-[4-(6-fluoro-2-tetrahydrofuran-2-yl-3H-imidazo[4,5-b]pyridin-7-yl)-1-piperidyl]methanone

Example 383 was prepared following a procedure similar to that of Examples 355 and 356 using tetrahydrofuran-2-carbaldehyde in step 6, and 2-amino-4-(pentafluoro-λ⁶-sulfanyl)benzoic acid and triethylamine instead of DIPEA in step 8.

### Example 384: (rac)-[4-(6-fluoro-2-tetrahydrofuran-2-yl-3H-imidazo[4,5-b]pyridin-7-yl)-1-piperidyl]-[4-(pentafluoro-λ⁶-sulfanyl)phenyl]methanone

Example 384 was prepared following a procedure similar to that of Examples 355 and 356 using tetrahydrofuran-2-carbaldehyde in step 6, and 4-(pentafluoro-λ⁶-sulfanyl)benzoic acid and triethylamine instead of DIPEA in step 8.

### Example 385: [4-(6-fluoro-2-tetrahydropyran-4-yl-3H-imidazo[4,5-b]pyridin-7-yl)-1-piperidyl]-[2-hydroxy-4-(pentafluoro-λ⁶-sulfanyl)phenyl]methanone

Example 385 was prepared following a procedure similar to that of Example 131 using 2-hydroxy-4-(pentafluoro-λ⁶-sulfanyl)benzoic acid and 6-fluoro-7-(4-piperidyl)-2-tetrahydropyran-4-yl-3H-imidazo[4,5-b]pyridine;dihydrochloride (described in step 7 of Example 350). LC/MS (m/z, M+H): 551; ¹H NMR (400 MHz, DMSO-d6, 100° C.) δ ppm 1.79 (br d, J=12.5 Hz, 2H), 1.85-2.00 (m, 4H), 2.19-2.37 (partially hidden m, 2H), 3.05 (partially hidden br t, J=12.3 Hz, 2H), 3.11-3.20 (m, 1H), 3.41-3.62 (m, 3H), 3.95 (dt, J=11.5, 3.5 Hz, 2H), 4.02-4.41 (m, 2H), 7.21-7.44 (m, 4H), 8.11 (d, J=3.4 Hz, 1H), 11.74-13.15 (m, 1H)

### Example 386: (rac)-[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone

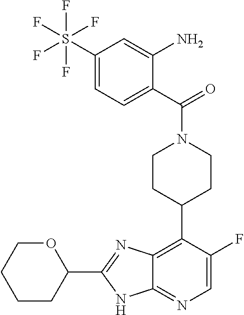 [SMILES: Nc1cc(S(F)(F)(F)(F)F)ccc1C(=O)N1CCC(c2c(F)cnc3[nH]c(C4CCCCO4)nc23)CC1]

### Step 1: (rac)-tert-butyl 4-(6-fluoro-2-tetrahydropyran-2-yl-3H-imidazo[4,5-b]pyridin-7-yl)piperidine-1-carboxylate

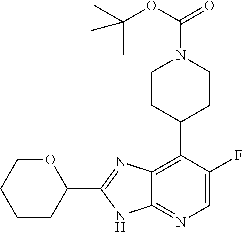 [SMILES: CC(C)(C)OC(=O)N1CCC(c2c(F)cnc3[nH]c(C4CCCCO4)nc23)CC1]

Under argon atmosphere, to a solution of tetrahydropyran-2-carboxylic acid (63 mg, 0.48 mmol) in MeCN (6 mL) were successively added tert-butyl-4-(2,3-diamino-5-fluoro-4-pyridyl)piperidine-1-carboxylate (described in step 5 of Examples 355 and 356; 150 mg, 0.48 mmol), 1-methylimidazole (139 mg, 1.69 mmol), and N,N,N′,N′-tetramethylchloroformamidinium hexafluorophosphate (163 mg, 0.58 mmol), and the resulting reaction mixture was stirred at room temperature for 15 hours. After 15 hours, 0.2 equivalents of tetrahydropyran-2-carboxylic acid and 0.2 equivalents of N,N,N′,N′-tetramethylchloroformamidinium hexafluorophosphate were added and the whole mixture was stirred at room temperature for 3 additional hours, then stirred at 80° C. for one hour. Then, the reaction mixture was cooled down to room temperature, and filtered to give 122 mg of a white powder. This latter was triturated in DCM, filtered, washed with water, dried in vacuo. The resulting solid was finally purified on silica gel (SiO₂ 4 g), eluting with DCM/AcOEt 100/0 to 50/50 to give 55 mg (28% yield) of tert-butyl 4-(6-fluoro-2-tetrahydropyran-2-yl-3H-imidazo[4,5-b]pyridin-7-yl)piperidine-1-carboxylate as a pale brown powder.

### Step 2: (rac)-6-fluoro-7-(4-piperidyl)-2-tetrahydropyran-2-yl-3H-imidazo[4,5-b]pyridine;hydrochloride

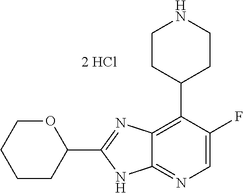 [SMILES: Fc1cnc2[nH]c(C3CCCCO3)nc2c1C1CCNCC1]

Step 2 was performed following the procedure used in step 7 of Examples 355 and 356 to give 50 mg (100% yield) of 6-fluoro-7-(4-piperidyl)-2-tetrahydropyran-2-yl-3H-imidazo[4,5-b]pyridine;hydrochloride as a white powder.

### Step 3: (rac)-[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone

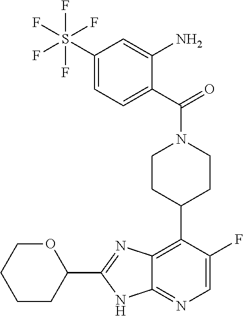 [SMILES: Nc1cc(S(F)(F)(F)(F)F)ccc1C(=O)N1CCC(c2c(F)cnc3[nH]c(C4CCCCO4)nc23)CC1]

Step 3 was performed following the protocol described in Example 131 using 6-fluoro-7-(4-piperidyl)-2-tetrahydropyran-2-yl-3H-imidazo[4,5-b]pyridine;hydrochloride and 2-amino-4-(pentafluoro-λ⁶-sulfanyl)benzoic acid to give 34 mg (47% yield) of (rac)-[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone as a white solid. LC/MS (m/z, M+H, Method 1): calc. 550.2, found 550.1.

### Example 387: [2-amino-4-(pentafluoro-λ⁶-sulfanyl)phenyl]-[4-[2-[4-(hydroxymethyl)-1-piperidyl]-3H-imidazo[4,5-b]pyridin-7-yl]-1-piperidyl]methanone;formic acid

Example 387 was prepared by a procedure similar to that of step 3 of Example 174 using 4-piperidylmethanol and tert-butyl 4-(2-bromo-3H-imidazo[4,5-b]pyridin-7-yl)piperidine-1-carboxylate (prepared in step 2 of Example 174), followed by a procedure similar to that of steps 7 and 8 of Examples 355 and 356 using 2-amino-4-(pentafluoro-λ⁶-sulfanyl)benzoic acid in step 8. LC/MS (m/z, M+H): 561; ¹H NMR (400 MHz, DMSO-d6, 100° C.) δ ppm 1.17-1.32 (m, 2H), 1.60-1.70 (m, 1H), 1.72-1.80 (m, 2H), 1.80-1.93 (m, 4H), 2.95-3.16 (partially hidden m, 3H), 3.22-3.38 (m, 1H), 3.32 (d, J=6.1 Hz, 2H), 4.03-4.24 (m, 4H), 5.45 (br s, 2H), 6.79 (d, J=5.4 Hz, 1H), 6.99 (dd, J=8.4, 2.3 Hz, 1H), 7.22 (d, J=8.4 Hz, 1H), 7.27 (d, J=2.3 Hz, 1H), 7.82 (br d, J=5.4 Hz, 1H), 11.10-12.27 (m, 1H)

### Example 388: [4-[6-fluoro-2-(3-methoxycyclobutyl)-3H-imidazo[4,5-b]pyridin-7-yl]-1-piperidyl]-[4-(pentafluoro-λ⁶-sulfanyl)phenyl]methanone

Example 388 was prepared by a procedure similar to that of step 2 of Example 364 using 3-methoxycyclobutanecarboxylic acid and tert-butyl 4-(2,3-diamino-5-fluoro-4-pyridyl)piperidine-1-carboxylate (described in step 5 of Examples 355 and 356), followed by a procedure similar to that of steps 7 and 8 of Examples 355 and 356 using 4-(pentafluoro-λ⁶-sulfanyl)benzoic acid in step 8.

### Example 389: (rac)-[4-(6-fluoro-2-tetrahydropyran-2-yl-3H-imidazo[4,5-b]pyridin-7-yl)-1-piperidyl]-[4-(pentafluoro-λ⁶-sulfanyl)phenyl]methanone

Example 389 was prepared by a procedure similar to that of Example 386 using 4-(pentafluorothio)benzoic acid in step 3.

### Example 390: (trans)-[4-[6-fluoro-2-(4-hydroxycyclohexyl)-3H-imidazo[4,5-b]pyridin-7-yl]-1-piperidyl]-[4-(pentafluoro-λ⁶-sulfanyl)phenyl]methanone

Example 390 was prepared by a procedure similar to that of Example 386 using Intermediate III in step 1 and 4-(pentafluorothio)benzoic acid in step 3. LC/MS (m/z, M+H): 549; ¹H NMR (400 MHz, DMSO-d6, 100° C.) δ ppm 1.26-1.42 (m, 2H), 1.68 (qd, J=12.5, 3.3 Hz, 2H), 1.81 (br d, J=12.5 Hz, 2H), 1.92-2.02 (m, 2H), 2.02-2.13 (m, 2H), 2.22-2.40 (partially hidden m, 2H), 2.80 (tt, J=11.4, 3.7 Hz, 1H), 3.11 (t, J=12.5 Hz, 2H), 3.42-3.65 (m, 2H), 3.89-4.50 (m, 3H), 7.64 (br d, J=8.5 Hz, 2H), 7.95 (br d, J=8.5 Hz, 2H), 8.04-8.16 (m, 1H), 12.35-12.58 (m, 1H)

### Example 391: [2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone

Example 391 was prepared following a procedure similar to that of Examples 355 and 356 using 3-methoxycyclobutane-1-carbaldehyde in step 6.

### Example 392: [4-[6-fluoro-2-[(1R,4R)-2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone

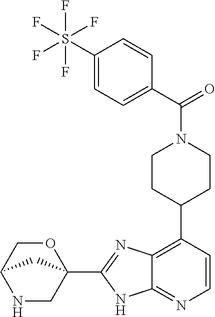 [SMILES: O=C(c1ccc(S(F)(F)(F)(F)F)cc1)N1CCC(c2ccnc3[nH]c([C@@]45CN[C@@H](CO4)C5)nc23)CC1]

To a suspension of [4-(2,3-diamino-5-fluoro-4-pyridyl)-1-piperidyl]-[4-(pentafluoro-λ⁶-sulfanyl)phenyl]methanone (as prepared in step 1 of Example 408; 75 mg, 0.16 mmol) in dry DCM (4 mL) were added (1R,4R)-5-(tert-butoxycarbonyl)2-oxa-5-azabicyclo[2.2.1]heptane-1-carboxylic acid (44 mg, 0.17 mmol) and triethylamine (0.06 mL, 0.41 mmol). The resulting mixture was cooled to 0° C., and 2-chloro-methylpyridinium iodide (47 mg, 0.18 mmol) was added and the mixture was stirred for 5 minutes at 0° C. followed by 2.5 hours at room temperature, then refluxed 1 hour. 3.5 mg of (1R,4R)-5-(tert-butoxycarbonyl)2-oxa-5azabicyclo[2.2.1]heptane-1-carboxylic acid, 4.8 μL of triethylamine and 3.8 mg of 2-chloro-methylpyridinium iodide were added. The mixture was stirred at room temperature for 20 minutes and then at reflux for one hour. Then, the reaction mixture was eluted through a SCX column with first MeOH then a mixture MeOH/NH₃-MeOH 2M and finally NH₃/MeOH 2M. The resulting filtrate was concentrated in vacuo, and the resulting residue was diluted with 0.5 mL of DCM and 0.5 mL of 2M HCl/Et₂O. The resulting mixture was stirred at room temperature for 30 minutes, concentrated in vacuo and the resulting residue was eluted through a SCX (5 g) column first with MeOH then a mixture MeOH/NH₃-MeOH 2M (1/1) and finally NH₃/MeOH 2M. The fraction obtained with NH₃ solution were put together and evaporated to give a brown oil. The obtained crude material was purified on silica gel (SiO₂ 10 g) eluting with DCM/7M NH₃-MeOH 4.5% to 6% to give 38 mg (43% yield) of [4-[6-fluoro-2-[(1R,4R)-2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone as a white solid. LC/MS (m/z, M+H, Method 1): calc. 548.2, found 548.4; ¹H NMR (400 MHz, DMSO-d6, 100° C.) δ ppm 1.82 (br d, J=12.4 Hz, 2H), 2.04-2.15 (m, 2H), 2.19-2.28 (m, 2H), 3.12 (t, J=12.8 Hz, 2H), 3.22 (d, J=10.4 Hz, 1H), 3.35-3.43 (m, 2H), 3.61 (tt, J=12.1, 3.6 Hz, 1H), 3.73-3.76 (m, 1H), 3.87 (br d, J=6.9 Hz, 1H), 3.99 (br d, J=6.9 Hz, 1H), 4.03-4.30 (m, 2H), 7.64 (br d, J=8.9 Hz, 2H), 7.95 (d, J=8.9 Hz, 2H), 8.17 (br d, J=3.3 Hz, 1H)

### Examples 393-396: [4-[2-[3-methoxycyclohexyl]-3H-imidazo[4,5-b]pyridin-7-yl]-1-piperidyl]-[4-(pentafluoro-λ⁶-sulfanyl)phenyl]methanone, Isomers 1, 2, 3, and 4

Examples 393-397 were prepared by a procedure similar to that of step 1 of Example 5 using 3-methoxycyclohexanecarboxylic acid and tert-butyl 4-(2,3-diamino-4-pyridyl)piperidine-1-carboxylate (described in step 2 of Examples 1 and 2), followed by a procedure similar to that of step 3 of Example 29, followed by a procedure similar to that of step 8 of Examples 355 and 356 using 4-(pentafluoro-λ⁶-sulfanyl)benzoic acid, followed by isomeric separation using Method CS2, Method CS19, and Method CS12 (with Chiralpak AY column (5 μm, 250×30 mm), eluting with (n-Heptane 95% EtOH 5%)+0.1% TEA (flow rate 45 mL/min)), successively.

### Example 397: (rac)-[4-(6-fluoro-2-thiomorpholin-2-yl-3H-imidazo[4,5-b]pyridin-7-yl)-1-piperidyl]-[4-(pentafluoro-λ⁶-sulfanyl)phenyl]methanone

Example 397 was prepared by a procedure similar to that of Example 364 using 4-(tert-butoxycarbonyl)thiomorpholine-2-carboxylic acid in step 2.

### Example 398: (trans)-[4-[6-fluoro-2-(4-methoxycyclohexyl)-3H-imidazo[4,5-b]pyridin-7-yl]-1-piperidyl]-[2-hydroxy-4-(pentafluoro-λ⁶-sulfanyl)phenyl]methanone

Example 398 was prepared by a procedure similar to that of step 8 of Examples 355 and 356 using 2-hydroxy-4-(pentafluoro-λ⁶-sulfanyl)benzoic acid. LC/MS (m/z, M+H): 579; ¹H NMR (400 MHz, DMSO-d6, 100° C.) δ ppm 1.22-1.39 (m, 2H), 1.62-1.75 (m, 2H), 1.79 (br d, J=11.5 Hz, 2H), 2.05-2.17 (m, 4H), 2.19-2.29 (m, 2H), 2.78-2.87 (partially hidden m, 1H), 3.01-3.12 (partially hidden m, 2H), 3.21 (tt, J=10.0, 4.0 Hz, 1H), 3.28 (s, 3H), 3.44-3.61 (m, 1H), 3.91-4.28 (m, 2H), 7.27-7.43 (m, 3H), 8.09 (d, J=3.3 Hz, 1H)

### Example 399: [2-amino-4-(pentafluoro-λ⁶-sulfanyl)phenyl]-[4-[6-fluoro-2-(1-methoxy-1-methyl-ethyl)-3H-imidazo[4,5-b]pyridin-7-yl]-1-piperidyl]methanone

Example 399 was prepared following a procedure similar to that of Examples 355 and 356 using 2-methoxy-2-methyl-propanal in step 6. LC/MS (m/z, M+H): 538; ¹H NMR (400 MHz, DMSO-d6, 100° C.) δ ppm 1.62 (s, 6H), 1.82 (br d, J=11.1 Hz, 2H), 2.28-2.41 (m, 2H), 3.05-3.18 (m, 2H), 3.11 (s, 3H), 3.51-3.68 (m, 1H), 4.08-4.25 (m, 2H), 5.45 (br s, 2H), 7.01 (dd, J=8.4, 2.2 Hz, 1H), 7.22 (br d, J=8.4 Hz, 1H), 7.28 (d, J=2.2 Hz, 1H), 8.16 (br d, J=3.0 Hz, 1H), 12.53-12.85 (m, 1H)

### Examples 400 and 401: [4-[6-fluoro-2-[tetrahydrofuran-3-yl]-3H-imidazo[4,5-b]pyridin-7-yl]-1-piperidyl]-[4-(pentafluoro-λ⁶-sulfanyl)phenyl]methanone, Isomers 1 and 2

Examples 400 and 401 were obtained by chiral separation of the compound of Example 374 using Method CS21.

### Example 402: [4-[6-fluoro-2-[(2R,6R)-6-methylmorpholin-2-yl]-3H-imidazo[4,5-b]pyridin-7-yl]-1-piperidyl]-[4-(pentafluoro-λ⁶-sulfanyl)phenyl]methanone;dihydrochloride

Example 402 was prepared by a procedure similar to that of step 2 of Example 364 using (2R,6R)-4-(tert-butoxycarbonyl)-6-methylmorpholine-2-carboxylic acid and [4-(2,3-diamino-5-fluoro-4-pyridyl)-1-piperidyl]-[4-(pentafluoro-λ⁶-sulfanyl)phenyl]methanone (described in step 1 of Example 364), followed by a procedure similar to that of steps 2 and 3 of Example 378.

Examples 403 and 404: [2-amino-4-(pentafluoro-λ⁶-sulfanyl)phenyl]-[4-[6-fluoro-2-[tetrahydrofuran-3-yl]-3H-imidazo[4,5-b]pyridin-7-yl]-1-piperidyl]methanone, Isomers 1 and 2

Examples 403 and 404 were obtained by chiral separation of the compound of Example 377 using Method CS21.

Example 403 (Isomer 1): LC/MS (m/z, M+H, Method 2): 536; ¹H NMR (400 MHz, DMSO-d6, 100° C.) δ ppm 1.76-1.86 (m, 2H), 2.27-2.41 (m, 4H), 3.05-3.15 (m, 2H), 3.46-3.60 (m, 1H), 3.67 (quin, J=7.4 Hz, 1H), 3.79-3.87 (m, 1H), 3.89-4.01 (m, 2H), 4.05-4.26 (m, 3H), 5.46 (br s, 2H), 7.01 (dd, J=8.4, 2.3 Hz, 1H), 7.22 (br d, J=8.4 Hz, 1H), 7.28 (d, J=2.3 Hz, 1H), 8.12 (br d, J=2.9 Hz, 1H), 12.32-12.85 (m, 1H)

Example 404 (Isomer 2): LC/MS (m/z, M+H, Method 2): 536; ¹H NMR (400 MHz, DMSO-d6, 100° C.) δ ppm 1.77-1.86 (m, 2H), 2.20-2.38 (m, 4H), 3.04-3.15 (m, 2H), 3.53 (tt, J=12.1, 3.5 Hz, 1H), 3.67 (quin, J=7.4 Hz, 1H), 3.79-3.87 (m, 1H), 3.89-3.99 (m, 2H), 4.06-4.21 (m, 3H), 5.45 (s, 2H), 7.01 (dd, J=8.5, 2.3 Hz, 1H), 7.22 (d, J=8.5 Hz, 1H), 7.28 (d, J=2.3 Hz, 1H), 8.12 (d, J=3.4 Hz, 1H), 11.62-13.45 (m, 1H)

### Examples 405 and 406: [4-[6-fluoro-2-[morpholin-2-yl]-3H-imidazo[4,5-b]pyridin-7-yl]-1-piperidyl]-[4-(pentafluoro-λ⁶-sulfanyl)phenyl]methanone, Isomers 1 and 2

Examples 405 and 406 were obtained by chiral separation of the compound of Example 364 using Method CS21.

Example 405 (Isomer 1): LC/MS (m/z, M+H, Method 2): 536; ¹H NMR (400 MHz, DMSO-d6, 100° C.) δ ppm 1.77-1.88 (m, 2H), 2.18-2.28 (m, 2H), 2.79-2.84 (m, 2H), 2.97-3.05 (m, 2H), 3.06-3.26 (m, 4H), 3.49-3.75 (m, 3H), 3.82-3.93 (m, 1H), 3.98-4.41 (m, 2H), 4.69 (dd, J=9.6, 2.9 Hz, 1H), 7.65 (br d, J=8.5 Hz, 2H), 7.95 (d, J=8.5 Hz, 2H), 8.17 (d, J=3.4 Hz, 1H)

### Example 407: (rac)-5-[6-fluoro-7-[1-[4-(pentafluoro-λ⁶-sulfanyl)benzoyl]-4-piperidyl]-3H-imidazo[4,5-b]pyridin-2-yl]piperidin-2-one

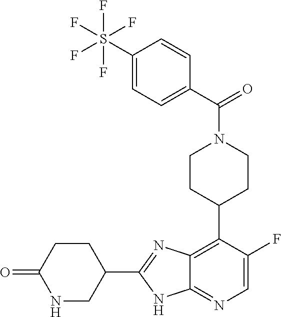 [SMILES: O=C1CCC(c2nc3c(C4CCN(C(=O)c5ccc(S(F)(F)(F)(F)F)cc5)CC4)c(F)cnc3[nH]2)CN1]

### Step 1: (rac)-N-[3-amino-5-fluoro-4-[1-[4-(pentafluoro-λ⁶-sulfanyl)benzoyl]-4-piperidyl]-2-pyridyl]-6-oxo-piperidine-3-carboxamide

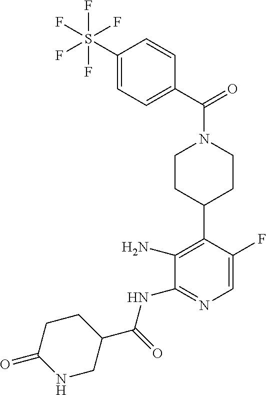 [SMILES: Nc1c(NC(=O)C2CCC(=O)NC2)ncc(F)c1C1CCN(C(=O)c2ccc(S(F)(F)(F)(F)F)cc2)CC1]

To a suspension of [4-(2,3-diamino-5-fluoro-4-pyridyl)-1-piperidyl]-[4-(pentafluoro-λ⁶-sulfanyl)phenyl]methanone (described in step 1 of Example 364; 120 mg, 0.27 mmol) in dry DCM (4 mL) was added 6-oxopiperidine-3-carboxylic acid (12 mg, 0.08 mmol), triethylamine (0.095 mL, 0.68 mmol) and 2-chloro-methylpyridinium iodide (86 mg, 0.33 mmol) and the mixture was refluxed for 3 hours then stirred at room temperature for 12 hours. After 12 hours, 6-oxopiperidine-3-carboxylic acid (12 mg, 0.08 mmol), triethylamine (22.9 μL, 0.16 mmol) and 2-chloro-methylpyridinium iodide (22 mg, 0.08 mmol) were added and the resulting mixture was heated at reflux for 2.5 hours. Then the DCM solution was taken to a SCX cartridge (25 g) eluted with MeOH then a mixture MeOH/NH₃-MeOH 2M and finally NH₃/MeOH 2M to give 154 mg (100% yield) of crude (rac)-N-[3-amino-5-fluoro-4-[1-[4-(pentafluoro-λ⁶-sulfanyl)benzoyl]-4-piperidyl]-2-pyridyl]-6-oxo-piperidine-3-carboxamide as a yellow oil which was used in the next step without further purification.

### Step 2: (rac)-5-[6-fluoro-7-[1-[4-(pentafluoro-λ⁶-sulfanyl)benzoyl]-4-piperidyl]-3H-imidazo[4,5-b]pyridin-2-yl]piperidin-2-one

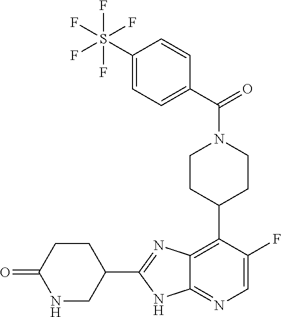 [SMILES: O=C1CCC(c2nc3c(C4CCN(C(=O)c5ccc(S(F)(F)(F)(F)F)cc5)CC4)c(F)cnc3[nH]2)CN1]

To a solution of (rac)-N-[3-amino-5-fluoro-4-[1-[4-(pentafluoro-λ⁶-sulfanyl)benzoyl]-4-piperidyl]-2-pyridyl]-6-oxo-piperidine-3-carboxamide (154 mg, 0.27 mmol) in dry CH₃CN (15 mL) was added acetic acid (0.15 mL) and the mixture was stirred at reflux for 4 hours. Then, the reaction mixture was cooled down to room temperature, concentrated to dryness, diluted with DCM and washed first with water and then with a saturated aqueous solution of NaCl. The organic layer was separated, dried over MgSO₄, filtered and evaporated to give 145 mg of a brown oil. The crude material was purified on silica gel (SiO₂ 15 g) eluting with a mixture DCM/MeOH 4% to 6% to give 51 mg (34% yield over two steps) of (rac)-5-[6-fluoro-7-[1-[4-(pentafluoro-λ⁶-sulfanyl)benzoyl]-4-piperidyl]-3H-imidazo[4,5-b]pyridin-2-yl]piperidin-2-one as a white powder. LC/MS (m/z, M+H, Method 1): calc. 548.1, found 548.4.

### Example 408: (trans)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone

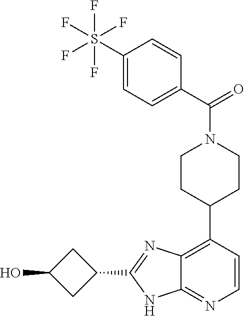 [SMILES: O=C(c1ccc(S(F)(F)(F)(F)F)cc1)N1CCC(c2ccnc3[nH]c([C@H]4C[C@H](O)C4)nc23)CC1]

### Step 1: [4-(2,3-diamino-4-pyridyl)-1-piperidyl]-[4-(pentafluoro-λ⁶-sulfanyl)phenyl]methanone

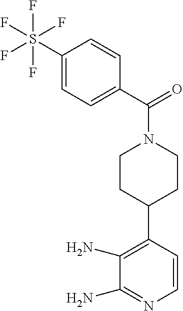 [SMILES: Nc1nccc(C2CCN(C(=O)c3ccc(S(F)(F)(F)(F)F)cc3)CC2)c1N]

To a solution of 4-(4-piperidyl)pyridine-2,3-diamine;hydrochloride (500 mg, 1.88 mmol) in DMF (4 mL) was added DIEA (1.88 mL, 7.54 mmol). The reaction mixture was stirred for 10 minutes at 0° C. and then, 4-(pentafluorothio)benzoic acid (492 mg, 1.8855 mmol) and TATU (639 mg, 1.88 mmol) were added. The reaction mixture was stirred for 30 minutes, then diluted with water and AcOEt, and transferred to a separating funnel. The aqueous layer was extracted with AcOEt and the combined organic layers were dried over magnesium sulfate, filtered and concentrated in vacuo. The resulting residue was purified on silica gel (SiO₂ 40 g) eluting with DCM/MeOH/NH₄OH 90/10/1 to give 320 mg (40% yield) of [4-(2,3-diamino-4-pyridyl)-1-piperidyl]-[4-(pentafluoro-λ⁶-sulfanyl)phenyl]methanone as a pale brown solid.

### Step 2: (trans)-[4-[2-[3-[tert-butyl(dimethyl)silyl]oxycyclobutyl]-3H-imidazo[4,5-b]pyridin-7-yl]-1-piperidyl]-[4-(pentafluoro-λ⁶-sulfanyl)phenyl]methanone

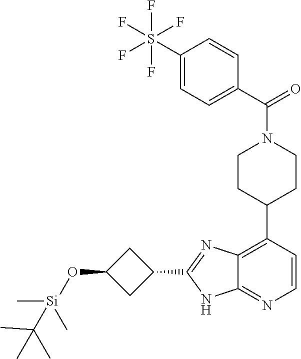 [SMILES: CC(C)(C)[Si](C)(C)O[C@H]1C[C@H](c2nc3c(C4CCN(C(=O)c5ccc(S(F)(F)(F)(F)F)cc5)CC4)ccnc3[nH]2)C1]

Step 2 was performed using the protocol described in step 1 of Example 252 using [4-(2,3-diamino-4-pyridyl)-1-piperidyl]-[4-(pentafluoro-λ⁶-sulfanyl)phenyl]methanone (previous step) and 3-[tert-butyl(dimethyl)silyl]oxycyclobutanecarboxylic acid (prepared following the protocol for the preparation of Intermediate I from trans-3-hydroxycyclobutanecarboxylic acid) to give 100 mg (68% yield) of (trans)-[4-[2-[3-[tert-butyl(dimethyl)silyl]oxycyclobutyl]-3H-imidazo[4,5-b]pyridin-7-yl]-1-piperidyl]-[4-(pentafluoro-λ⁶-sulfanyl)phenyl]methanone as a white powder.

### Step 3: (trans)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone

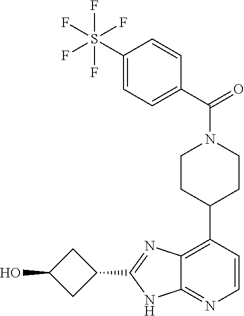 [SMILES: O=C(c1ccc(S(F)(F)(F)(F)F)cc1)N1CCC(c2ccnc3[nH]c([C@H]4C[C@H](O)C4)nc23)CC1]

To a solution of [4-[2-[3-[tert-butyl(dimethyl)silyl]oxycyclobutyl]-3H-imidazo[4,5-b]pyridin-7-yl]-1-piperidyl]-[4-(pentafluoro-λ⁶-sulfanyl)phenyl]methanone (100 mg, 0.16 mmol) in MeOH (2 mL) was added a 2 M HCl solution in EtOH (0.32 mL, 0.65 mmol) and the resulting reaction mixture was stirred at room temperature for 2 hours. Then, the reaction mixture was concentrated in vacuo, and filtered through an SCX cartridge (2 g) eluted with MeOH followed by a 2 N solution of NH₃ in MeOH. The filtrate was concentrated in vacuo to give 43 mg (52% yield) of (trans)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone as a white solid. LC/MS (m/z, M+H, Method 1): calc. 503.1, found 503.4.

### Example 409: [2-amino-4-(pentafluoro-λ⁶-sulfanyl)phenyl]-[4-(2-cyclopentyl-6-fluoro-3H-imidazo[4,5-b]pyridin-7-yl)-1-piperidyl]methanone

Example 409 was prepared following a procedure similar to that of steps 6-8 of Examples 355 and 356 using cyclopentanecarbaldehyde in step 6 and 2-amino-4-(pentafluoro-λ⁶-sulfanyl)benzoic acid in step 8. LC/MS (m/z, M+H): 564; ¹H NMR (400 MHz, DMSO-d6, 100° C.) δ ppm 1.62-1.73 (m, 2H), 1.73-1.86 (m, 4H), 1.88-2.00 (m, 2H), 2.03-2.13 (m, 2H), 2.18-2.39 (m, 2H), 3.09 (br t, J=12.1 Hz, 2H), 3.30 (quin, J=8.0 Hz, 1H), 3.47-3.60 (m, 1H), 4.08-4.24 (m, 2H), 5.45 (br s, 2H), 7.01 (dd, J=8.4, 2.3 Hz, 1H), 7.22 (d, J=8.4 Hz, 1H), 7.28 (d, J=2.3 Hz, 1H), 8.08 (br d, J=3.1 Hz, 1H), 12.22-12.73 (m, 1H)

### Examples 410-413: [2-amino-4-(pentafluoro-λ⁶-sulfanyl)phenyl]-[4-[2-[3-methoxycyclohexyl]-3H-imidazo[4,5-b]pyridin-7-yl]-1-piperidyl]methanone, Isomers 1, 2, 3, and 4

Examples 410-413 were prepared by a procedure similar to that of step 1 of Example 5 using 3-methoxycyclohexanecarboxylic acid and tert-butyl 4-(2,3-diamino-4-pyridyl)piperidine-1-carboxylate (described in step 2 of Examples 1 and 2), followed by a procedure similar to that of step 3 of Example 29, followed by a procedure similar to that of step 8 of Examples 355 and 356 using 2-amino-4-(pentafluoro-λ⁶-sulfanyl)benzoic acid, followed by isomeric separation using Method CS2, Method CS10 (column (5 μm, 250×30 mm), eluting with (n-heptane 90% EtOH 10%)+0.1% TEA (flow rate 40 mL)), and Method CS20, successively.

### Examples 414 and 415: [4-[6-fluoro-2-[oxepan-4-yl]-3H-imidazo[4,5-b]pyridin-7-yl]-1-piperidyl]-[4-(pentafluoro-λ⁶-sulfanyl)phenyl]methanone, Isomers 1 and 2

Examples 414 and 415 were obtained by chiral separation of the compound of Example 376 using Method CS15.

### Examples 416 and 417: [2-amino-4-(pentafluoro-λ⁶-sulfanyl)phenyl]-[4-[6-fluoro-2-[oxepan-4-yl]-3H-imidazo[4,5-b]pyridin-7-yl]-1-piperidyl]methanone, Isomers 1 and 2

Examples 416 and 417 were obtained by chiral separation of the compound of Example 375 using Method CS15.

Example 416 (Isomer 1): LC/MS (m/z, M+H, Method 2): 564; ¹H NMR (400 MHz, DMSO-d6, 100° C.) δ ppm 1.74-2.08 (m, 5H), 2.09-2.22 (m, 3H), 2.25-2.44 (m, 2H), 3.08-3.18 (m, 2H), 3.18-3.27 (m, 1H), 3.52-3.63 (m, 1H), 3.63-3.91 (m, 4H), 4.11-4.26 (m, 2H), 5.47 (br s, 2H), 7.04 (dd, J=8.5, 2.3 Hz, 1H), 7.26 (d, J=8.5 Hz, 1H), 7.31 (d, J=2.3 Hz, 1H), 8.12 (br s, J=1.4 Hz, 1H), 12.26-12.73 (m, 1H)

### Examples 418 and 419: [2-amino-4-(pentafluoro-λ⁶-sulfanyl)phenyl]-[4-[6-fluoro-2-[tetrahydrofuran-2-yl]-3H-imidazo[4,5-b]pyridin-7-yl]-1-piperidyl]methanone, Isomers 1 and 2

Examples 418 and 419 were obtained by chiral separation of the compound of Example 383 using Method CS5.

Example 418 (Isomer 1): LC/MS (m/z, M+H, Method 2): 536; ¹H NMR (400 MHz, DMSO-d6, acetic acid-d4, 100° C.) δ ppm 1.82 (br d, J=12.3 Hz, 2H), 1.93-2.11 (m, 2H), 2.20-2.38 (m, 4H), 3.10 (br t, J=12.5 Hz, 2H), 3.57 (tt, J=12.1, 3.8 Hz, 1H), 3.82-3.90 (m, 1H), 3.96-4.05 (m, 1H), 4.08-4.22 (m, 2H), 5.07 (dd, J=7.3, 6.4 Hz, 1H), 7.02 (dd, J=8.5, 2.3 Hz, 1H), 7.23 (br d, J=8.5 Hz, 1H), 7.28 (d, J=2.3 Hz, 1H), 8.15 (d, J=3.3 Hz, 1H)

### Example 420 and 421: [4-[6-fluoro-2-[tetrahydrofuran-2-yl]-3H-imidazo[4,5-b]pyridin-7-yl]-1-piperidyl]-[4-(pentafluoro-λ⁶-sulfanyl)phenyl]methanone, Isomers 1 and 2

Examples 420 and 421 were obtained by chiral separation of the compound of Example 384 using Method CS5.

Example 420 (Isomer 1): LC/MS (m/z, M+H, Method 2): 521; ¹H NMR (400 MHz, DMSO-d6, acetic acid-d4, 100° C.) δ ppm 1.77-1.85 (m, 2H), 1.92-2.13 (m, 2H), 2.17-2.38 (m, 4H), 3.12 (br t, J=12.4 Hz, 2H), 3.59 (tt, J=12.2, 3.8 Hz, 2H), 3.80-3.92 (m, 1H), 3.96-4.06 (m, 1H), 4.06-4.36 (m, 2H), 5.08 (dd, J=7.3, 6.4 Hz, 1H), 7.64 (br d, J=8.5 Hz, 2H), 7.95 (br d, J=8.5 Hz, 2H), 8.16 (d, J=3.4 Hz, 1H)

### Example 422: [4-[6-fluoro-2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone

### Intermediate V: 3-[tert-butyl(dimethyl)silyl]oxybicyclo[1.1.1]pentane-1-carbaldehyde

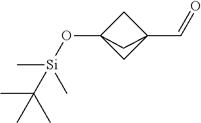

### Step 1: [3-[tert-butyl(dimethyl)silyl]oxy-1-bicyclo[1.1.1]pentanyl]methanol

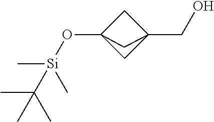

To a solution of Intermediate IV (345 mg, 1.42 mmol) in THF (3 mL) was added at 0° C. a 1 M solution of LiAlH₄ in THF (2.85 mL, 2.85 mmol). The reaction mixture was then warmed up to room temperature and stirred for 2.5 hours. Then, the reaction mixture was cooled down to 0° C. and hydrolyzed with a saturated aqueous solution of sodium sulfate, then warmed up to room temperature and stirred for 30 minutes. The whole was then filtered, washed with THF and concentrated in vacuo to give 289 mg (89% yield) of [3-[tert-butyl(dimethyl)silyl]oxy-1-bicyclo[1.1.1]pentanyl]methanol as a colorless oil.

### Step 2: 3-[tert-butyl(dimethyl)silyl]oxybicyclo[1.1.1]pentane-1-carbaldehyde

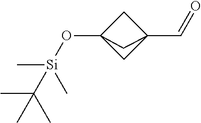

To a solution [3-[tert-butyl(dimethyl)silyl]oxy-1-bicyclo[1.1.1]pentanyl]methanol (280 mg, 1.22 mmol) in DCM (6 mL) was added portionwise at 0° C., Dess-Martin periodinane (624 mg, 1.47 mmol). The reaction mixture was stirred at 0° C. for 10 minutes followed by 2 hours at room temperature, then diluted with DCM and with 6 mL of an aqueous saturated solution of NaHCO₃, 3 mL of a 5% aqueous solution of Na₂S₂O₃. The whole was stirred and then filtered on a PTFE cartridge. The organic layer was concentrated in vacuo to give a residue which was triturated in cold diethyl ether, and filtered. The filtrate was concentrated in vacuo to give 271 mg (98% yield) of 3-[tert-butyl(dimethyl)silyl]oxybicyclo[1.1.1]pentane-1-carbaldehyde as a yellow oil which was used in the next step without further purification.

Example 422 was prepared following a procedure similar to that of step 6 of Examples 355 and 356 using Intermediate V and [4-(2,3-diamino-5-fluoro-4-pyridyl)-1-piperidyl]-[4-(pentafluoro-λ⁶-sulfanyl)phenyl]methanone (described in step 1 of Example 364), followed by a procedure similar to that of step 7 of Examples 355 and 356.

### Example 423: [2-amino-4-(pentafluoro-λ⁶-sulfanyl)phenyl]-[4-[6-fluoro-2-(3-hydroxy-1-bicyclo[1.1.1]pentanyl)-3H-imidazo[4,5-b]pyridin-7-yl]-1-piperidyl]methanone

Example 423 was prepared following a procedure similar to that of step 6 of Examples 355 and 356 using Intermediate V and [2-amino-4-(pentafluoro-λ⁶-sulfanyl)phenyl]-[4-(2,3-diamino-5-fluoro-4-pyridyl)-1-piperidyl]methanone (prepared as described in step 1 of Example 364 using 2-amino-4-(pentafluoro-λ⁶-sulfanyl)benzoic acid), followed by a procedure similar to that of step 7 of Examples 355 and 356. LC/MS (m/z, M+H, Method 2): 548; ¹H NMR (400 MHz, DMSO-d6, 100° C.) δ ppm 1.75-1.86 (m, 2H), 2.19-2.30 (m, 2H), 2.25 (s, 6H), 3.04-3.15 (m, 2H), 3.48-3.57 (m, 1H), 4.07-4.23 (m, 2H), 5.32-5.56 (m, 2H), 5.94-6.30 (m, 1H), 7.01 (dd, J=8.4, 2.3 Hz, 1H), 7.22 (d, J=8.4 Hz, 1H), 7.29 (d, J=2.3 Hz, 1H), 8.11 (d, J=3.4 Hz, 1H), 12.42-12.90 (m, 1H)

### Examples 424: (trans)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone

Example 424 was prepared by a procedure similar to that of step 1 of Example 5 using [2-amino-4-(pentafluoro-λ⁶-sulfanyl)phenyl]-[4-(2,3-diamino-4-pyridyl)-1-piperidyl]methanone (described in step 1 of Example 408), followed by a procedure similar to that of step 7 of Examples 355 and 356.

### Example 425: [4-(2-cyclopentyl-6-fluoro-3H-imidazo[4,5-b]pyridin-7-yl)-1-piperidyl]-[4-(pentafluoro-λ⁶-sulfanyl)phenyl]methanone

Example 425 was prepared following a procedure similar to that of steps 6-8 of Examples 355 and 356 using cyclopentanecarbaldehyde and tert-butyl 4-(2,3-diamino-5-fluoro-4-pyridyl)piperidine-1-carboxylate (described in step 5 of Examples 355 and 356) and using 4-(pentafluoro-λ⁶-sulfanyl)benzoic acid in step 8.

### Example 426: [4-[6-fluoro-2-(3-methoxy-1-bicyclo[1.1.1]pentanyl)-3H-imidazo[4,5-b]pyridin-7-yl]-1-piperidyl]-[4-(trifluoromethoxy)phenyl]methanone

Example 426 was prepared following a procedure similar to that of Example 429 using 3-methoxybicyclo[1.1.1]pentane-1-carbaldehyde in step 3. LC/MS (m/z, M+H, Method 2): 505; ¹H NMR (400 MHz, DMSO-d6, 100° C.) δ ppm 1.80 (br d, J=12.0 Hz, 2H), 2.17-2.38 (m, 2H), 2.31 (s, 6H), 3.10 (br t, J=12.3 Hz, 2H), 3.29 (s, 3H), 3.58 (br t, J=11.0 Hz, 1H), 4.06-4.30 (m, 2H), 7.40 (br d, J=8.8 Hz, 2H), 7.51-7.61 (m, 2H), 8.13 (br d, J=3.3 Hz, 1H), 12.63-12.93 (m, 1H)

### Example 427: [4-[6-fluoro-2-(3-methoxy-1-bicyclo[1.1.1]pentanyl)-3H-imidazo[4,5-b]pyridin-7-yl]-1-piperidyl]-[2-hydroxy-4-(pentafluoro-λ⁶-sulfanyl)phenyl]methanone

Example 427 was prepared following a procedure similar to that of Example 131 using 2-hydroxy-4-(pentafluoro-λ⁶-sulfanyl)benzoic acid and 6-fluoro-2-(3-methoxy-1-bicyclo[1.1.1]pentanyl)-7-(4-piperidyl)-3H-imidazo[4,5-b]pyridine;hydrochloride (prepared as described in the second step of Examples 381 and 382) LC/MS (m/z, M+H, Method 2): 563; ¹H NMR (400 MHz, DMSO-d6, 100° C.) δ ppm 1.79 (br d, J=13.4 Hz, 2H), 2.15-2.27 (m, 2H), 2.31 (s, 6H), 3.06-3.14 (partially hidden m, 2H), 3.29 (s, 3H), 3.49-3.61 (m, 1H), 3.88-4.36 (m, 2H), 7.30-7.42 (m, 3H), 8.13 (d, J=3.5 Hz, 1H), 9.98-10.61 (m, 1H)

### Example 428: [2-amino-4-(trifluoromethoxy)phenyl]-[4-[6-fluoro-2-(3-methoxy-1-bicyclo[1.1.1]pentanyl)-3H-imidazo[4,5-b]pyridin-7-yl]-1-piperidyl]methanone

Example 428 was prepared following a procedure similar to that of Example 451 using 3-methoxybicyclo[1.1.1]pentane-1-carbaldehyde in step 2. LC/MS (m/z, M+H, Method 2): 520; ¹H NMR (400 MHz, DMSO-d6, 100° C.) δ ppm 1.80 (br d, J=12.8 Hz, 2H) 2.31 (s, 8H) 3.08 (br t, J=12.7 Hz, 2H) 3.29 (s, 3H) 3.43-3.67 (m, 1H) 4.17 (br d, J=11.8 Hz, 2H) 5.33 (br s, 2H) 6.49 (br d, J=8.4 Hz, 1H) 6.70 (br s, 1H) 7.13 (d, J=8.4 Hz, 1H) 8.12 (br d, J=2.8 Hz, 1H) 12.50-12.96 (m, 1H)

### Example 429 [4-[6-fluoro-2-(2-oxabicyclo[2.2.2]octan-4-yl)-3H-imidazo[4,5-b]pyridin-7-yl]-1-piperidyl]-[4-(trifluoromethoxy)phenyl]methanone

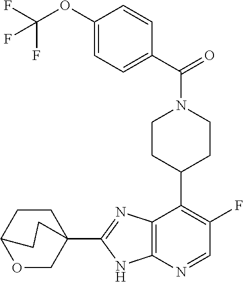 [SMILES: O=C(c1ccc(OC(F)(F)F)cc1)N1CCC(c2c(F)cnc3[nH]c(C45CCC(CC4)OC5)nc23)CC1]

### Step 1: 5-Fluoro-4-(4-piperidyl)pyridine-2,3-diamine;hydrochloride

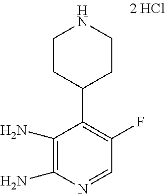 [SMILES: Nc1ncc(F)c(C2CCNCC2)c1N]

To a solution of tert-butyl 4-(2,3-diamino-5-fluoro-4-pyridyl)piperidine-1-carboxylate (742 mg, 2.39 mmol) in MeOH (20 mL) was added 5.98 mL of a 4 N solution of HCl in dioxane. The resulting reaction mixture was stirred for 4 hours at room temperature, then concentrated in vacuo to give 677 mg (100% yield) of 5-fluoro-4-(4-piperidyl)pyridine-2,3-diamine;hydrochloride as an orange solid which was used in the next step without further purification.

### Step 2: [4-(2,3-diamino-5-fluoro-4-pyridyl)-1-piperidyl]-[4-(trifluoromethoxy)phenyl]methanone

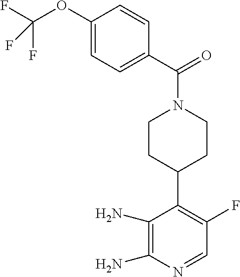 [SMILES: Nc1ncc(F)c(C2CCN(C(=O)c3ccc(OC(F)(F)F)cc3)CC2)c1N]

To a solution of 5-fluoro-4-(4-piperidyl)pyridine-2,3-diamine;hydrochloride (338 mg, 1.19 mmol) in DMF (5 mL) wad added DIPEA (617 mg, 0.83 mL, 4.77 mmol). Then, 4-(trifluoromethoxy)benzoic acid (246 mg, 1.19 mmol) and TBTU (421 mg, 1.31 mmol) were added and the resulting mixture was stirred at room temperature for 1 hour. After one hour, the reaction mixture was diluted with ethyl acetate, washed with an aqueous saturated solution of NaHCO₃, water and brine. The organic layer was then dried over sodium sulfate, filtered and concentrated in vacuo. The resulting residue was purified on silica gel (SiO₂ 24 g) eluting with DCM/MeOH/NH₄OH 94/6/0.6 to give 400 mg (84% yield) of [4-(2,3-diamino-5-fluoro-4-pyridyl)-1-piperidyl]-[4-(trifluoromethoxy)phenyl]methanone.

### Step 3: [4-[6-Fluoro-2-(2-oxabicyclo[2.2.2]octan-4-yl)-3H-imidazo[4,5-b]pyridin-7-yl]-1-piperidyl]-[4-(trifluoromethoxy)phenyl]methanone

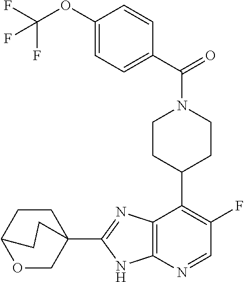 [SMILES: O=C(c1ccc(OC(F)(F)F)cc1)N1CCC(c2c(F)cnc3[nH]c(C45CCC(CC4)OC5)nc23)CC1]

In a microwave vial, were successively introduced [4-(2,3-diamino-5-fluoro-4-pyridyl)-1-piperidyl]-[4-(trifluoromethoxy)phenyl]methanone (75 mg, 0.19 mmol), 2-oxabicyclo[2.2.2]octane-4-carbaldehyde (29 mg, 0.21 mmol) and DMF (2 mL). To this solution, was then added sodium metabisulfite (46 mg, 0.24 mmol). The vial was then sealed and submitted to microwave at 125° C. for 90 minutes. After 90 minutes, the vial was cooled down to room temperature, opened, diluted with AcOEt, transferred to a separating funnel, and extracted twice with AcOEt. The combined organic layers were then washed with water, dried over magnesium sulfate, filtered, and concentrated in vacuo. The resulting residue was purified by flash chromatography to give 70 mg (72% yield) of [4-[6-fluoro-2-(2-oxabicyclo[2.2.2]octan-4-yl)-3H-imidazo[4,5-b]pyridin-7-yl]-1-piperidyl]-[4-(trifluoromethoxy)phenyl]methanone. LC/MS (m/z, M+H, Method 2): calc. 519.2, found 519.4; ¹H NMR (400 MHz, DMSO-d6, 100° C.) δ ppm 1.71-1.92 (m, 4H), 2.03-2.22 (m, 6H), 2.23-2.40 (m, 2H), 3.13 (br t, J=12.3 Hz, 2H), 3.59 (tt, J=12.0, 3.8 Hz, 1H), 3.84 (br s, 1H), 4.07 (s, 2H), 4.12-4.29 (m, 2H), 7.43 (d, J=8.5 Hz, 2H), 7.58 (d, J=8.5 Hz, 2H), 8.15 (d, J=3.3 Hz, 1H), 12.14-13.00 (m, 1H)

### Example 430: [2-amino-5-(pentafluoro-λ⁶-sulfanyl)phenyl]-[4-(2-tetrahydropyran-4-yl-3H-imidazo[4,5-b]pyridin-7-yl)-1-piperidyl]methanone

Example 430 was prepared following a procedure similar to that of step 3 of Example 293 using 2-amino-5-(pentafluoro-λ⁶-sulfanyl)benzoic acid and 7-(4-piperidyl)-2-tetrahydropyran-4-yl-3H-imidazo[4,5-b]pyridine;dihydrochloride (obtained by a procedure similar to step 2 of Example 12 using HCl instead of TFA).

### Example 431: [4-[2-(1-cyclopropyl-4-piperidyl)-6-fluoro-3H-imidazo[4,5-b]pyridin-7-yl]-1-piperidyl]-[4-(pentafluoro-λ⁶-sulfanyl)phenyl]methanone

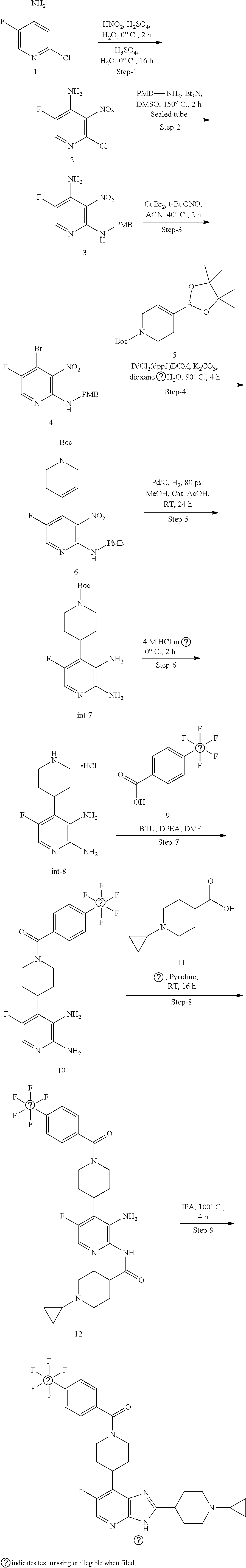

Step 1: To a stirred solution of 2-chloro-5-fluoro-pyridin-4-amine (20.0 g, 0.136 mol) in sulfuric acid (200.0 mL) was added potassium nitrate (27.6 g, 0.273 mol) at 0° C. The reaction mass was stirred at 0° C. for 1 hour. The reaction was monitored by TLC and LCMS. After completion, the reaction mass was allowed to room temperature. add ice water (100 mL) solid was formed. The solid was filtered and dried well to afford crude compound (20 g). The crude compound (20 g) in sulfuric acid (200.0 mL) was stirred at room temperature for 16 hours. The reaction was monitored by TLC and LCMS. The reaction mass was added to ice water (100 mL), solid was filtered and dried well to afford 2-chloro-5-fluoro-3-nitro-pyridin-4-amine (20.0 g, 0.104 mol, yield: 76.5%) as a yellow solid. LC/MS: t_{RA}=1.68 min, m/z: 189.9 [M−H]

Step 2: To a stirred solution of 2-chloro-5-fluoro-3-nitro-pyridin-4-amine (20.0 g, 0.104 mol) and N,N-diethylethanamine (31.7 g, 0.313 mol) in DMSO (200.0 mL) was followed by (4-methoxyphenyl)methanamine (28.6 g, 0.209 mol) at RT. The reaction mass was stirred at 160° C. for 2 hours. The reaction was monitored by TLC and LCMS. After completion of the reaction, the reaction mixture concentrated under reduced pressure to obtained crude mass, which was diluted with ice water (300 mL) and extracted with ethyl acetate (2×200 mL). Organic layers were separated, dried over Na₂SO₄, filtered, and concentrate under reduced pressure to afford crude mass. Then crude was purified by column chromatography to afford 5-fluoro-N2-[(4-methoxyphenyl)methyl]-3-nitro-pyridine-2,4-diamine (20.0 g, 0.0684 mol, yield: 65.5%) as a yellow solid. LC/MS: t_{RA}=1.68 min, m/z: 292 [M+H].

Step 3: To a stirred solution of dibromocopper (15.3 g, 0.0684 mol) and tert-butyl nitrite (14.1 g, 0.137 mol) in acetonitrile (200 mL) was followed by 5-fluoro-N2-[(4-methoxyphenyl)methyl]-3-nitro-pyridine-2,4-diamine (20.0 g, 0.0684 mol) at 45° C. The reaction mass was stirred at 45° C. for 2 hours under argon atmosphere. The reaction was monitored by TLC and LCMS. The reaction mass was concentrated under reduced pressure to obtained crude mass, which was diluted with 4 M aq. HCl (500 mL) and extracted with ethyl acetate (200 mL×2). Organic layers were separated, dried over Na₂SO₄, filtered and concentrate the reduced pressure to afford crude. The crude mass was purified by column to afford 4-bromo-5-fluoro-N-[(4-methoxyphenyl)methyl]-3-nitro-pyridin-2-amine (15.0 g, 0.0421 mol, yield: 61.5%) as a yellow solid. LC/MS: t_{RA}=1.95 min, m/z: 356 [M+H].

Step 4: To a stirred solution of tert-butyl 4-(4,4,5,5-tetramethyl-1,3,2-dioxaborolan-2-yl)-3,6-dihydro-2H-pyridine-1-carboxylate (15.0 g, 0.0485 mol) and 4-bromo-5-fluoro-N-[(4-methoxyphenyl)methyl]-3-nitro-pyridin-2-amine (34.6 g, 0.0970 mol) in 1,4-dioxane (150 mL) and water (50.0 mL) was added K₂CO₃ (13.4 g, 0.0970 mol) and were degassed under argon for 15 min. Then was added PdCl₂(dppf). DCM complex (3.55 g, 0.00485 mol) and the reaction mixture was stirred at 90° C. for 16 hours. The progress of the reaction was monitored by TLC and LCMS. After completion of the reaction, reaction mass filtered through celite bed. The filtrate was diluted with ethyl acetate (500 mL), washed with water (500 mL), separate the organic layers and dried over anhydrous Na₂SO₄ and concentrated under reduced pressure. The crude compound (30 g) was adsorbed over silica gel and purified by Gravity column to afford the desired product of tert-butyl 4-[5-fluoro-2-[(4-methoxyphenyl)methylamino]-3-nitro-4-pyridyl]-3,6-dihydro-2H-pyridine-1-carboxylate (15.0 g, 0.0327 mol, yield: 67.4%) as a yellow gummy liquid. LC/MS: t_{RA}=2.63 min, m/z: 459 [M+H].

Step 5: To a stirred solution of tert-butyl 4-[5-fluoro-2-[(4-methoxyphenyl)methylamino]-3-nitro-4-pyridyl]-3,6-dihydro-2H-pyridine-1-carboxylate (10.0 g, 0.0218 mol) in methanol (200 mL) was added 10% Pd/C (2.32 g, 50% wet) and add catalytic amount of acetic acid (1.31 g, 0.0218 mol) under an inert atmosphere and then the reaction mixture was stirred overnight under a 100 psi under hydrogen atmosphere in parr shaker at room temperature for 16 hours. The progress of the reaction was monitored by TLC and LCMS. The reaction mixture was filtered through celite bed and washed with ethanol and concentrated to dryness. Crude compound (9 g) was adsorbed over silica gel and purified by Gravity Column Chromatography to afford tert-butyl 4-(2,3-diamino-5-fluoro-4-pyridyl)piperidine-1-carboxylate (4.00 g, 0.0129 mol, yield: 59.1%) as a yellow solid. LC/MS: t_{RA}=1.67 min, m/z: 311 [M+H].

Step 6: To a stirred solution of tert-butyl 4-(2,3-diamino-5-fluoro-4-pyridyl)piperidine-1-carboxylate (1.00 g, 0.00322 mol) in dichloromethane (10.0 mL) was followed by 4M hydrogen chloride in dioxane (8.05 mL, 0.0322 mol) under argon atmosphere and then the reaction mixture was stirred at room temperature for 1 hour. The progress of the reaction was monitored by TLC and LCMS. reaction mass was concentrated under reduced pressure to obtain crude mass which was triturated with diethyl ether (50 mL) and decant the solvent and the solid was dried well to afford 5-fluoro-4-(4-piperidyl)pyridine-2,3-diamine (1 g, 0.00476 mol, yield: 148%) as an off white solid.

Step 7: To a stirred solution of 4-(pentafluoro-λ⁶-sulfanyl)benzoic acid (1.00 g, 0.00403 mol) and N,N-diethylethanamine (1.22 g, 0.0121 mol), [benzotriazol-1-yloxy(dimethylamino)methylene]-dimethyl-ammonium;tetrafluoroborate (1.94 g, 0.00604 mol) in N,N-dimethylformamide (10.0 mL) was followed by 5-fluoro-4-(4-piperidyl)pyridine-2,3-diamine (0.847 g, 0.00403 mol) at 0° C. The reaction mass was stirred at RT for 1 hour under argon atmosphere. The reaction was monitored by TLC and LCMS. The reaction mixture was concentrated under reduced pressure to obtained crude mass, which was diluted with ice water (100 mL) and extracted with ethyl acetate (100 mL×2). Organic layers was separated, dried over Na₂SO₄, filtered and concentrate the reduced pressure to afford crude. Crude mass was purified by Column Chromatography to afford [4-(2,3-diamino-5-fluoro-4-pyridyl)-1-piperidyl]-[4-(pentafluoro-λ⁶-sulfanyl)phenyl]methanone (1.00 g, 0.00227 mol, yield: 56.4%) as a yellow solid. LC/MS: t_{RA}=1.57 min, m/z: 441 [M+H].

Step 8: To a stirred solution of 1-cyclopropylpiperidine-4-carboxylic acid (0.0768 g, 0.000454 mol) and [4-(2,3-diamino-5-fluoro-4-pyridyl)-1-piperidyl]-[4-(pentafluoro-λ⁶-sulfanyl)phenyl]methanone (0.100 g, 0.000227 mol) in pyridine (2.00 mL) was followed by 3-(ethyliminomethyleneamino)-N,N-dimethyl-propan-1-amine;hydrochloride (0.0653 g, 0.000341 mol) at 0° C. and the reaction mass was stirred at RT for 2 hours. The reaction was monitored by TLC and LCMS. The reaction mass was concentrated under reduced pressure to obtained crude mass, which was diluted with water (100 mL) and extracted with ethyl acetate (100 mL). Organic layers was separated, dried over Na₂SO₄, filtered and concentrate the reduced pressure to afford crude compound which was purified by column to afford N-[3-amino-5-fluoro-4-[1-[4-(pentafluoro-lambda6-sulfanyl)benzoyl]-4-piperidyl]-2-pyridyl]-1-cyclopropyl-piperidine-4-carboxamide (0.100 g, 0.000101 mol, yield: 44.7%) as an off white solid. LC/MS: t_{RA}=1.70 min, m/z: 592 [M+H].

Step 9: To a stirred solution of N-[3-amino-5-fluoro-4-[1-[4-(pentafluoro-λ⁶-sulfanyl)benzoyl]-4-piperidyl]-2-pyridyl]-1-cyclopropyl-piperidine-4-carboxamide (100 mg, 0.100 mol) in propan-2-ol (2.00 mL) was followed by K₂CO₃ (13.8 mg, 0.100 mol) at rt and the reaction mass was stirred at 100° C. for 5 h. The reaction was monitored by TLC and LCMS. The reaction mixture was concentrated under reduced pressure to obtained crude mass, which was diluted with water (100 mL) and extracted with ethyl acetate (100 mL). Organic layers was separated, dried over Na₂SO₄, filtered and concentrate the reduced pressure to afford crude which was purified by preparative HPLC to afford [4-[2-(1-cyclopropyl-4-piperidyl)-6-fluoro-3H-imidazo[4,5-b]pyridin-7-yl]-1-piperidyl]-[4-(pentafluoro-λ⁶-sulfanyl)phenyl]methanone (17 mg, 2.96e−5 mol, yield: 29%) as an off white solid. LC/MS: t_{RA}=1.58 min, m/z: 574 [M+H]; HPLC: t_{RA}=5.72 min, 99.02%; ¹H NMR (400 MHz, DMSO-d₆) δ ppm 8.16 (s, 1H) 7.99-8.06 (m, 2H) 7.64-7.72 (m, 2H) 4.59-4.74 (m, 1H) 3.51-3.67 (m, 2H) 2.98-3.09 (m, 2H) 2.80-2.97 (m, 2H) 2.23-2.35 (m, 4H) 1.91-1.99 (m, 2H) 1.74-1.88 (m, 3H) 1.58-1.73 (m, 2H) 0.38-0.48 (m, 2H) 0.26-0.35 (m, 2H)

### Example 432: [2-amino-4-(trifluoromethoxy)phenyl]-[4-[6-fluoro-2-[3-(trifluoromethyl)-1-bicyclo[1.1.1]pentanyl]-3H-imidazo[4,5-b]pyridin-7-yl]-1-piperidyl]methanone

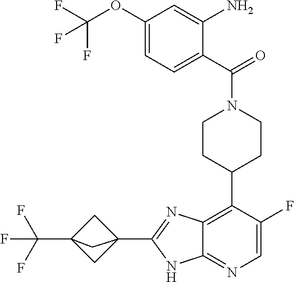 [SMILES: Nc1cc(OC(F)(F)F)ccc1C(=O)N1CCC(c2c(F)cnc3[nH]c(C45CC(C(F)(F)F)(C4)C5)nc23)CC1]

### Step 1: tert-butyl 4-[6-fluoro-2-[3-(trifluoromethyl)-1-bicyclo[1.1.1]pentanyl]-3H-imidazo[4,5-b]pyridin-7-yl]piperidine-1-carboxylate

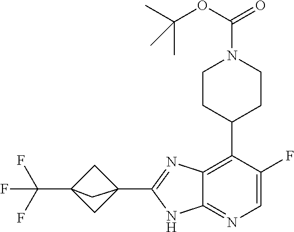 [SMILES: CC(C)(C)OC(=O)N1CCC(c2c(F)cnc3[nH]c(C45CC(C(F)(F)F)(C4)C5)nc23)CC1]

At 0° C., to a solution of tert-butyl 4-(2,3-diamino-5-fluoro-4-pyridyl)piperidine-1-carboxylate (described in step 5 of Examples 355 and 356; 100 mg, 0.32 mmol) in DMF (5 mL) were successively added 3-(trifluoromethyl)bicyclo[1.1.1]pentane-1-carboxylic acid (58 mg, 0.32 mmol), triethylamine (225 μL, 1.61 mmol) and TATU (156 mg, 0.48 mmol). The resulting mixture was stirred for 3 hours at room temperature. After 3 hours, the reaction mixture was concentrated in vacuo (bath temperature 55° C.), then diluted with AcOEt and an aqueous saturated solution of NaHCO₃. The aqueous layer was extracted three times with AcOEt and the combined organic layers were dried over magnesium sulfate, filtered and concentrated. The resulting residue was purified on silica gel (SiO₂ 12 g) eluting with DCM/MeOH 90/10 to give 95 mg (43% yield) of tert-butyl 4-[6-fluoro-2-[3-(trifluoromethyl)-1-bicyclo[1.1.1]pentanyl]-3H-imidazo[4,5-b]pyridin-7-yl]piperidine-1-carboxylate as a white powder.

### Step 2: 6-fluoro-7-(4-piperidyl)-2-[3-(trifluoromethyl)-1-bicyclo[1.1.1]pentanyl]-3H-imidazo[4,5-b]pyridine;hydrochloride

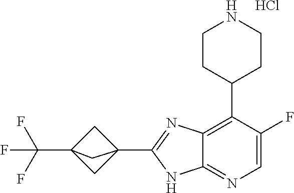 [SMILES: Cl ; Fc1cnc2[nH]c(C34CC(C(F)(F)F)(C3)C4)nc2c1C1CCNCC1]

Step 2 was performed using the protocol described in step 7 of Examples 355 and 356 to give 82 mg (100% yield) of 6-fluoro-7-(4-piperidyl)-2-[3-(trifluoromethyl)-1-bicyclo[1.1.1]pentanyl]-3H-imidazo[4,5-b]pyridine;hydrochloride as a white powder.

### Step 3: [2-amino-4-(trifluoromethoxy)phenyl]-[4-[6-fluoro-2-[3-(trifluoromethyl)-1-bicyclo[1.1.1]pentanyl]-3H-imidazo[4,5-b]pyridin-7-yl]-1-piperidyl]methanone

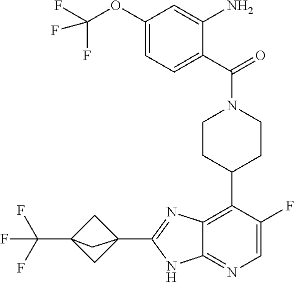 [SMILES: Nc1cc(OC(F)(F)F)ccc1C(=O)N1CCC(c2c(F)cnc3[nH]c(C45CC(C(F)(F)F)(C4)C5)nc23)CC1]

Step 3 was performed following the protocol described in step 8 of Examples 355 and 356, using 2-amino-4-(trifluoromethoxy)benzoic acid and triethylamine instead of DIPEA, to give 64 mg (62% yield) of [2-amino-4-(trifluoromethoxy)phenyl]-[4-[6-fluoro-2-[3-(trifluoromethyl)-1-bicyclo[1.1.1]pentanyl]-3H-imidazo[4,5-b]pyridin-7-yl]-1-piperidyl]methanone as a white solid. LC/MS (m/z, M+H, Method 1): calc. 558.2, found 558.4; ¹H NMR (400 MHz, DMSO-d6, 30° C.) δ ppm 1.77 (br d, J=11.8 Hz, 2H), 2.06-2.30 (m, 2H), 2.46 (s, 6H), 2.91-3.25 (m, 2H), 3.48-3.64 (m, 1H), 3.73-4.74 (m, 2H), 5.50-5.65 (m, 2H), 6.52 (br dd, J=8.4, 1.2 Hz, 1H), 6.68 (br s, 1H), 7.13 (d, J=8.4 Hz, 1H), 8.23 (br d, J=2.8 Hz, 1H), 13.20-13.41 (m, 1H)

### Examples 433 and 434: [2-amino-4-(pentafluoro-λ⁶-sulfanyl)phenyl]-[4-[6-fluoro-2-[tetrahydropyran-2-yl]-3H-imidazo[4,5-b]pyridin-7-yl]-1-piperidyl]methanone, Isomers 1 and 2

Examples 433 and 434 were obtained by chiral separation of the compound of Example 386 using Method CS10.

Example 433 (Isomer 1): LC/MS (m/z, M+H, Method 2): 550; ¹H NMR (400 MHz, DMSO-d6, 100° C.) δ ppm 1.55-1.75 (m, 2H), 1.77-2.08 (m, 6H), 2.16-2.36 (m, 2H), 3.10 (br t, J=12.5 Hz, 2H), 3.51-3.70 (m, 2H), 3.95-4.06 (m, 1H), 4.08-4.25 (m, 2H), 4.65 (dd, J=10.1, 2.6 Hz, 1H), 5.45 (br s, 2H), 7.01 (dd, J=8.5, 2.3 Hz, 1H), 7.23 (d, J=8.5 Hz, 1H), 7.28 (d, J=2.3 Hz, 1H), 8.15 (br d, J=2.9 Hz, 1H), 12.26-12.96 (m, 1H)

### Example 435: (rac)-[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;hydrochloride

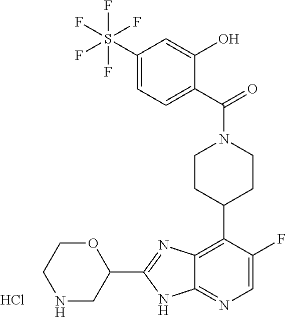 [SMILES: Cl ; O=C(c1ccc(S(F)(F)(F)(F)F)cc1O)N1CCC(c2c(F)cnc3[nH]c(C4CNCCO4)nc23)CC1]

### Step 1: [4-(2,3-diamino-5-fluoro-4-pyridyl)-1-piperidyl]-[2-hydroxy-4-(pentafluoro-λ⁶-sulfanyl)phenyl]methanone

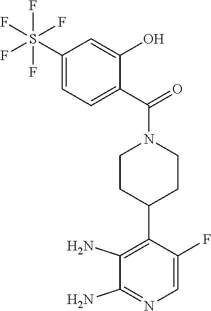 [SMILES: Nc1ncc(F)c(C2CCN(C(=O)c3ccc(S(F)(F)(F)(F)F)cc3O)CC2)c1N]

At room temperature, to a solution of 5-fluoro-4-(4-piperidyl)pyridine-2,3-diamine;dihydrochloride (step 1 example 74, 140 mg, 0.49 mmol) in DMF (1.5 mL) was added N,N-diisopropylethylamine (258 mg, 1.98 mmol). Then, were added a solution of 2-hydroxy-4-(pentafluoro-λ⁶-sulfanyl)benzoic acid (169 mg, 0.64 mmol) and TBTU (238 mg, 0.74 mmol) in DMF (1.5 mL) followed by N,N-diisopropylethylamine (64 mg, 0.49 mmol). The whole mixture was stirred for 12 hours at room temperature, then concentrated in vacuo. The resulting residue was purified on silica gel (SiO₂ 120 g) eluting with DCM/MeOH 95/5 to 90/10 to give 83 mg (37% yield) of [4-(2,3-diamino-5-fluoro-4-pyridyl)-1-piperidyl]-[2-hydroxy-4-(pentafluoro-λ⁶-sulfanyl)phenyl]methanone as a white powder.

### Step 2: tert-butyl 2-[6-fluoro-7-[1-[2-hydroxy-4-(pentafluoro-λ⁶-sulfanyl)benzoyl]-4-piperidyl]-3H-imidazo[4,5-b]pyridin-2-yl]morpholine-4-carboxylate

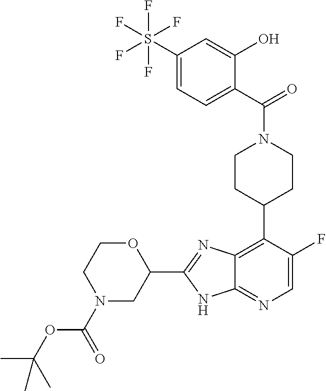 [SMILES: CC(C)(C)OC(=O)N1CCOC(c2nc3c(C4CCN(C(=O)c5ccc(S(F)(F)(F)(F)F)cc5O)CC4)c(F)cnc3[nH]2)C1]

Step 2 was performed following the protocol described in step 6 of Examples 355 and 356, using [4-(2,3-diamino-5-fluoro-4-pyridyl)-1-piperidyl]-[2-hydroxy-4-(pentafluoro-λ⁶-sulfanyl)phenyl]methanone and N-Boc-2-morpholinecarbaldehyde to give 37 mg (33% yield) of tert-butyl 2-[6-fluoro-7-[1-[2-hydroxy-4-(pentafluoro-λ⁶-sulfanyl)benzoyl]-4-piperidyl]-3H-imidazo[4,5-b]pyridin-2-yl]morpholine-4-carboxylate as a white solid.

### Step 3: (rac)-[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;hydrochloride

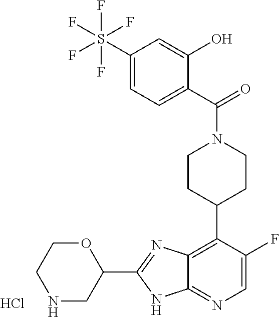 [SMILES: Cl ; O=C(c1ccc(S(F)(F)(F)(F)F)cc1O)N1CCC(c2c(F)cnc3[nH]c(C4CNCCO4)nc23)CC1]

At room temperature, 2-[6-fluoro-7-[1-[2-hydroxy-4-(pentafluoro-λ⁶-sulfanyl)benzoyl]-4-piperidyl]-3H-imidazo[4,5-b]pyridin-2-yl]morpholine-4-carboxylate (36 mg, 0.05 mmol) was solubilized in 2.5 mL of a 5 M HCl solution in i-PrOH. The whole mixture was stirred for one hour, concentrated to give 32 mg (100% yield) of (rac)-[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;hydrochloride as a yellow solid. LC/MS (m/z, M+H, Method 1): calc. 552.1, found 552.4; ¹H NMR (400 MHz, DMSO-d6, 100° C.) δ ppm 1.80 (br d, J=12.0 Hz, 2H) 2.18-2.31 (m, 2H) 2.85-3.38 (m partially hidden, 4H) 3.42-3.70 (m, 3H) 3.85-4.47 (m, 4H) 5.19 (dd, J=10.3, 2.8 Hz, 1H) 7.31-7.40 (m, 2H) 7.44 (d, J=1.9 Hz, 1H) 8.24 (d, J=3.4 Hz, 1H) 8.81-10.88 (m, 2H)

### Example 436: [4-[6-fluoro-2-[3-(trifluoromethyl)-1-bicyclo[1.1.1]pentanyl]-3H-imidazo[4,5-b]pyridin-7-yl]-1-piperidyl]-[4-(trifluoromethoxy)phenyl]methanone

Example 436 was prepared following a procedure similar to that of step 3 of Example 432 using 4-(trifluoromethoxy)benzoic acid. LC/MS (m/z, M+H, Method 2): 543; ¹H NMR (400 MHz, DMSO-d6 and acetic acid-d4, 100° C.) δ ppm 1.82 (br d, J=13.3 Hz, 2H), 2.17-2.32 (m, 2H), 2.47 (s, 6H), 3.05-3.18 (m, 2H), 3.59 (tt, J=12.0, 3.5 Hz, 1H), 4.09-4.32 (m, 2H), 7.39 (br d, J=8.6 Hz, 2H), 7.56 (br d, J=8.6 Hz, 2H), 8.17 (d, J=3.4 Hz, 1H)

### Example 437: [4-[2-(1-methoxy-1-methyl-ethyl)-3H-imidazo[4,5-b]pyridin-7-yl]-1-piperidyl]-[4-(trifluoromethoxy)phenyl]methanone

Example 437 was prepared following a procedure similar to that of step 3 of Example 429 using 2-methoxy-2-methylpropanal. LC/MS (m/z, M+H, Method 2): 463; ¹H NMR (400 MHz, DMSO-d6 and trifluoroacetic acid-d1, 100° C.) δ ppm 1.69 (s, 6H), 1.80-1.92 (m, 2H), 1.93-2.00 (m, 2H), 3.09-3.19 (m, 2H), 3.23 (s, 3H), 3.77 (tt, J=11.7, 3.7 Hz, 1H), 4.15-4.35 (m, 2H), 7.39 (br d, J=8.0 Hz, 2H), 7.54-7.64 (m, 3H), 8.52 (d, J=5.9 Hz, 1H)

### Examples 438 and 439: [4-[6-fluoro-2-[tetrahydropyran-2-yl]-3H-imidazo[4,5-b]pyridin-7-yl]-1-piperidyl]-[4-(pentafluoro-λ⁶-sulfanyl)phenyl]methanone, Isomers 1 and 2

Examples 438 and 439 were obtained by chiral separation of the compound of Example 389 using Method CS10.

### Examples 440 and 441: (trans)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone and (cis)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone

Examples 440 and 441 were prepared following a procedure similar to that of step 1 of Example 364 using 2-amino-4-(pentafluoro-λ⁶-sulfanyl)benzoic acid, followed by a procedure similar to that of step 2 of Examples 355 and 356 using a cis/trans mixture of 4-[tert-butyl(dimethyl)silyl]oxycyclohexanecarbaldehyde (prepared following the procedure described for the preparation of intermediate V, from intermediate III), followed by a procedure similar to that of step 7 of Examples 355 and 356 (with separation of cis/trans isomers during purification).

### Examples 442 and 443: (cis)-[4-[6-fluoro-2-(4-methoxycyclohexyl)-3H-imidazo[4,5-b]pyridin-7-yl]-1-piperidyl]-[4-(trifluoromethoxy)phenyl]methanone and (trans)-[4-[6-fluoro-2-(4-methoxycyclohexyl)-3H-imidazo[4,5-b]pyridin-7-yl]-1-piperidyl]-[4-(trifluoromethoxy)phenyl]methanone

Examples 442 and 443 were prepared following a procedure similar to that of step 3 of Example 429 using 4-methoxycyclohexanecarbaldehyde.

Example 442 (cis-isomer): LC/MS (m/z, M+H, Method 2): 521; ¹H NMR (400 MHz, DMSO-d6, 100° C.) δ ppm 1.55-1.65 (m, 2H), 1.75-1.85 (m, 4H), 1.85-1.95 (m, 2H), 1.95-2.09 (m, 2H), 2.19-2.42 (m, 2H), 2.89-3.00 (m hidden, 1H), 3.10 (br t, J=12.3 Hz, 2H), 3.26 (s, 3H), 3.40-3.48 (m, 1H), 3.55 (qd, J=8.0, 3.8 Hz, 1H), 4.05-4.33 (m, 2H), 7.39 (br d, J=8.1 Hz, 2H), 7.56 (br d, J=8.1 Hz, 2H), 8.09 (d, J=3.3 Hz, 1H), 12.02-12.78 (m, 1H)

Example 443 (trans-isomer): LC/MS (m/z, M+H, Method 2): 521.5; ¹H NMR (400 MHz, DMSO-d6, 100° C.) δ ppm 1.27-1.41 (m, 2H), 1.62-1.76 (m, 2H), 1.80 (br d, J=12.4 Hz, 2H), 2.12 (br d, J=10.4 Hz, 4H), 2.20-2.39 (m, 2H), 2.80-2.91 (m, 1H), 3.10 (br t, J=12.3 Hz, 2H), 3.17-3.25 (m, 1H), 3.28 (s, 3H), 3.48-3.63 (m, 1H), 4.08-4.31 (m, 2H), 7.39 (br d, J=8.1 Hz, 2H), 7.56 (br d, J=8.1 Hz, 2H), 8.10 (d, J=3.0 Hz, 1H), 12.03-12.83 (m, 1H)

### Example 444: [4-[6-fluoro-2-(1-methoxy-1-methyl-ethyl)-3H-imidazo[4,5-b]pyridin-7-yl]-1-piperidyl]-[4-(trifluoromethoxy)phenyl]methanone

Example 444 was prepared following a procedure similar to that of Example 429 using 2-methoxy-2-methylpropanal in step 3. LC/MS (m/z, M+H, Method 2): 481; ¹H NMR (400 MHz, DMSO-d6, 100° C.) δ ppm 1.62 (s, 6H), 1.82 (br d, J=12.6 Hz, 2H), 2.23-2.43 (m, 2H), 3.01-3.20 (m, 2H), 3.11 (s, 3H), 3.50-3.69 (m, 1H), 4.02-4.36 (m, 2H), 7.40 (br d, J=8.4 Hz, 2H), 7.55 (br d, J=8.4 Hz, 2H), 8.16 (br s, 1H), 12.33-12.98 (m, 1H)

### Example 445: (trans)-(4-(6-fluoro-2-(4-hydroxy-4-methylcyclohexyl)-3H-imidazo[4,5-b]pyridin-7-yl)piperidin-1-yl)(4-(pentafluoro-λ⁶-sulfanyl)phenyl)methanone

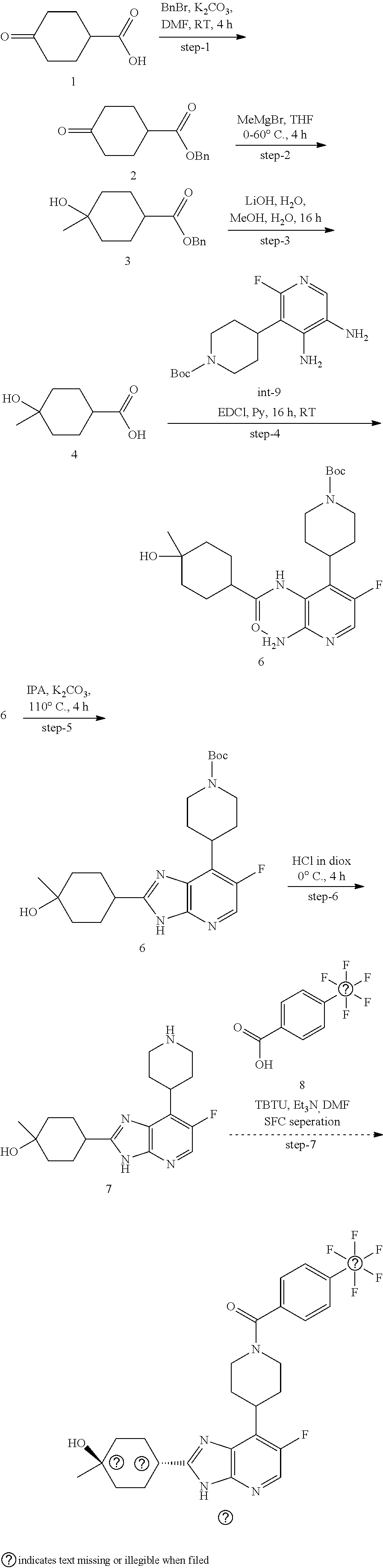

Step 1: To a stirred solution of 4-oxocyclohexanecarboxylic acid (10.0 g, 0.0703 mol) in DMF (50.0 mL), were added dipotassium carbonate (19.4 g, 0.141 mol) and bromomethylbenzene (24.1 g, 0.141 mol) at RT and stirred for 16 hours at RT. The reaction progress was monitored by TLC and LCMS. After completion (TLC and LC/MS), the reaction mixture was quenched with water (100 mL), extracted with ethyl acetate (2×50 mL), organic layer dried over sodium sulfate and concentrated under vacuum to get crude. The crude was purified by flash column in 10% of ethyl acetate/hexane as an eluent to afford benzyl 4-oxocyclohexanecarboxylate (11.0 g, 0.0474 mol, yield: 67.3%) as a light brown liquid. LC/MS: t_{RA}=3.443 min, m/z: 233.2 [M+H].

Step 2: To a stirred solution of benzyl 4-oxocyclohexanecarboxylate (10.0 g, 0.0431 mol) in tetrahydrofuran (50.0 mL), were added bromo(methyl)magnesium (1.00 mmol/L, 25.0 mL, 2.50e−5 mol) at 0° C. and then stirred at 65° C. for 3 hours. The reaction progress was monitored by TLC and LCMS. After completion (TLC and LC/MS), the reaction mixture was quenched with saturated ammonium chloride solution, extracted with ethyl acetate (2×20 mL), organic layers were dried over sodium sulphate and concentrated under vacuum to get crude mass which was purified by flash column in 30% ethyl acetate in hexane to afford benzyl 4-hydroxy-4-methyl-cyclohexanecarboxylate (2.00 g, 0.00805 mol, yield: 18.7%) as a colorless liquid.

Step 3: To a stirred solution of benzyl 4-hydroxy-4-methyl-cyclohexanecarboxylate (1.50 g, 0.00604 mol) in methanol (10.0 mL) and water (2.00 mL), was added lithium hydroxide (0.723 g, 0.0302 mol) at RT. The reaction mixture was heated to 60° C. for 4 hours and monitored by TLC and LCMS. After completion (TLC and LC/MS), the reaction mixture was concentrated under vacuum, diluted with water (10 mL), pH adjusted to acidic using dilute HCl, extracted with 10% MeOH/DCM (2×50 mL), combined organic layer washed with brine (50 mL), dried over sodium sulphate and concentrated under vacuum to obtain crude mass and recrystallized with acetonitrile and filtered the inorganic salt, concentrated the filtrate to afford 4-hydroxy-4-methyl-cyclohexanecarboxylic acid (1 g, 0.00822 mol, yield: 81%) as a colorless gummy. LC/MS: t_{RA}=0.306 min, m/z: 157 [M−H]

Step 4: To a stirred solution of 4-hydroxy-4-methyl-cyclohexanecarboxylic acid (0.250 g, 0.00158 mol) in pyridine (10.0 mL), was added tert-butyl 4-(2,3-diamino-5-fluoro-4-pyridyl) piperidine-1-carboxylate (0.490 g, 0.00158 mol) and cooled to 0° C., then 3-(ethyliminomethyleneamino)-N, N-dimethyl-propan-1-amine; hydrochloride (0.454 g, 0.00237 mol) was added at 0° C. and stirred for 16 hours at RT. The reaction progress was monitored by TLC and LCMS. After completion (TLC and LC/MS), the reaction mixture was diluted with water (10 mL), extracted with ethyl acetate (2×10 mL), Organic layer were dried over sodium sulphate and concentrated under vacuum to afford tert-butyl 4-[2-amino-5-fluoro-3-[(4-hydroxy-4-methyl-cyclohexanecarbonyl) amino]-4-pyridyl]piperidine-1-carboxylate (0.500 g, 0.00111 mol, yield: 70.2%). LC/MS: t_{RA}=1.713 min, m/z: 449.2 [M−H]

Step 5: To a stirred solution of tert-butyl 4-[2-amino-5-fluoro-3-[(4-hydroxy-4-methyl-cyclohexanecarbonyl) amino]-4-pyridyl]piperidine-1-carboxylate (0.500 g, 0.00111 mol) in propan-2-ol (10.00 mL) was added dipotassium carbonate (0.153 g, 0.00111 mol) at RT. The reaction mixture was stirred at 100° C. for 3 hours. After completion (TLC and LC/MS), the reaction mixture was monitored by TLC and LCMS. After completion (TLC and LC/MS), the reaction mixture was concentrated under vacuum, diluted with water (10 mL), extracted with ethyl acetate (2×10 mL), Organic layer were dried over sodium sulphate and concentrated under vacuum to get crude tert-butyl 4-[6-fluoro-2-(4-hydroxy-4-methyl-cyclohexyl)-3H-imidazo[4,5-b]pyridin-7-yl]piperidine-1-carboxylate (0.410 g, 0.000948 mol, yield: 85.4%). LC/MS: t_{RA}=1.63 min, m/z: 433.1 [M+H]

Step 6: To a stirred solution of tert-butyl 4-[6-fluoro-2-(4-hydroxy-4-methyl-cyclohexyl)-3H-imidazo[4,5-b]pyridin-7-yl]piperidine-1-carboxylate (0.400 g, 0.000925 mol) in dichloromethane (5.00 mL), was cooled to 0° C. and added hydrogen chloride in dioxane (5.00 mL, 0.000116 mol) at 0° C. and stirred for 3 hours at RT. The reaction Progress was monitored by TLC and LCMS. The reaction mixture was concentrated under vacuum and triturated with hexane to obtain crude mass 4-[6-fluoro-7-(4-piperidyl)-3H-imidazo[4,5-b]pyridin-2-yl]-1-methyl-cyclohexanol (80.0%, 0.350 g, 0.000842 mol, yield: 91.1%) as a brown solid. LC/MS: t_{RA}=0.19 min, m/z: 331.15 [M−H]

Step 7: To a stirred solution of 4-(pentafluoro-λ⁶-sulfanyl) benzoic acid (0.200 g, 0.000806 mol) in DMF (5.00 mL), was added [benzotriazol-1-yloxy(dimethylamino)methylene]-dimethyl-ammonium; tetrafluoroborate (0.388 g, 0.00121 mol), cooled to 0° C., then N, N-diethylethanamine (0.0815 g, 0.000806 mol), stirred for 5 min, 4-[6-fluoro-7-(4-piperidyl)-3H-imidazo[4,5-b]pyridin-2-yl]-1-methyl-cyclohexanol (0.321 g, 0.000967 mol). The reaction mixture was stirred at RT for 16 hours. The reaction mixture was monitored by TLC and LCMS. After completion (TLC and LC/MS), the reaction mixture was diluted with water (10 mL), extracted with ethyl acetate (2×10 mL), Organic layer were dried over sodium sulphate and concentrated under vacuum to get crude. The crude was purified by preparative HPLC (SAMPLE CODE: EXP-23-LJ66-049-C Mobile Phase: A=0.1% HCOOH IN WATER B=ACN Column: Gemini NX (250 mm×21.2 mm), 5.0p Flow: 18 ml/min Gradient Programmer: Time % B 0 20 2 30 8 60 PURITY>95%) pure fraction were concentrated to afford (trans)-[4-[6-fluoro-2-(4-hydroxy-4-methyl-cyclohexyl)-3H-imidazo[4,5-b]pyridin-7-yl]-1-piperidyl]-[4-(pentafluoro-λ⁶-sulfanyl)phenyl]methanone (0.0520 g, 9.24e−5 mol, yield: 11.5%) as a white solid. LC/MS: t_{RA}=1.798 min, m/z: 561.15 [M−H]. HPLC: t_{RA}=5.64 min, 95.7%¹H NMR (400 MHz, DMSO-d₆) δ ppm 8.13-8.20 (m, 1H) 7.98-8.06 (m, 2H) 7.61-7.70 (m, 2H) 4.62-4.78 (m, 1H) 4.31-4.39 (m, 1H) 3.47-3.69 (m, 3H) 2.80-3.02 (m, 3H) 1.90-2.07 (m, 3H) 1.60-1.90 (m, 8H) 1.40-1.57 (m, 3H)

### Examples 446 and 448: (cis)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone and (trans)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone

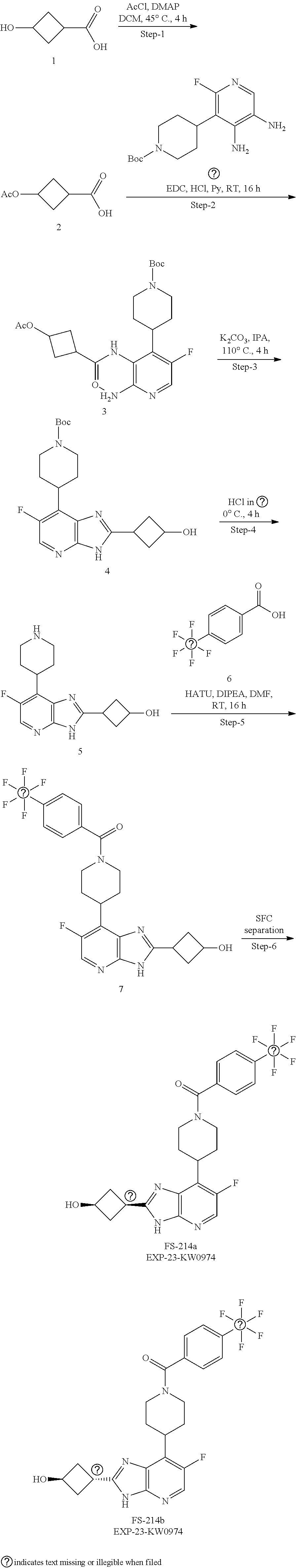

### Step 1: To a stirred solution of 3-hydroxycyclobutanecarboxylic acid (1.00 g, 0.00861 mol) in DCM (10.00 mL), were added N, N-dimethylpyridin-4-amine (0.105 g, 0.000861 mol) and acetyl chloride (1.01 g, 0.0129 mol) at RT and heated to 45° C. for 4 hours. The reaction progress was monitored by TLC and LCMS. After completion (TLC and LC/MS), the reaction mixture was quenched with water (10 mL), extracted with DCM (2×25 mL), organic layers were dried over sodium sulphate and concentrated under vacuum to obtain crude mass. The crude was purified by flash column in 40% ethyl acetate in hexane to afford 3-acetoxycyclobutanecarboxylic acid (1.10 g, 0.00696 mol, yield: 80.8%) as colorless gummy liquid. LC/MS: t_{RA}=0.690 min, m/z: 157.1 [M−H]

### Step 2: To a stirred solution of 3-acetoxycyclobutanecarboxylic acid (0.250 g, 0.00158 mol) in pyridine (10.0 mL), was added tert-butyl 4-(2,3-diamino-5-fluoro-4-pyridyl) piperidine-1-carboxylate (0.491 g, 0.00158 mol) and cooled to 0° C., then 2-(chloromethoxy) ethyl-trimethyl-silane (0.198 g, 0.00119 mol) was added at 0° C. and stirred for 16 hours at RT. The reaction mixture was monitored by TLC and LCMS. After completion (TLC and LC/MS), the reaction mixture was diluted with water (10 mL), extracted with ethyl acetate (2×10 mL), Organic layer were dried over sodium sulphate and concentrated under vacuum to afford tert-butyl 4-[3-[(3-acetoxycyclobutanecarbonyl) amino]-2-amino-5-fluoro-4-pyridyl]piperidine-1-carboxylate (50.0%, 0.500 g, 0.000555 mol, yield: 35.1%). LC/MS: t_{RA}=1.748 min, m/z: 449.15 [M−H]

Step 3: To a stirred solution of tert-butyl 4-[3-[(3-acetoxycyclobutanecarbonyl) amino]-2-amino-5-fluoro-4-pyridyl]piperidine-1-carboxylate (0.500 g, 0.00111 mol) in propan-2-ol (5.00 mL) was added dipotassium; carbonate (0.460 g, 0.00333 mol) at RT. The reaction mixture was stirred at 100° C. for 3 hours. The reaction mixture was monitored by TLC and LCMS. After completion (TLC and LC/MS), the reaction mixture was concentrated under vacuum, diluted with water (10 mL), extracted with ethyl acetate (2×10 mL), Organic layer were dried over sodium sulphate and concentrated under vacuum to get crude. LC/MS: t_{RA}=2.14 min, m/z: 389.15 [M−H]

Step 4: To a stirred solution of tert-butyl 4-[2-(3-acetoxycyclobutyl)-6-fluoro-3H-imidazo[4,5-b]pyridin-7-yl]piperidine-1-carboxylate (0.500 g, 0.00116 mol) in dichloromethane (5.00 mL), was cooled to 0° C. and added hydrogen chloride in dioxane (1M) (5.00 mL, 0.000116 mol) at 0° C. and stirred for 3 hours at RT. The reaction progress was monitored by TLC and LCMS. After completion (TLC and LC/MS), the reaction mixture was concentrated under vacuum and triturated with hexane to obtain crude mass 3-[6-fluoro-7-(4-piperidyl)-3H-imidazo[4,5-b]pyridin-2-yl]cyclobutanol (0.400 g, 0.00138 mol, yield: 119%). LC/MS: t_{RA}=0.203 min, m/z: 288.95 [M−H]

Step 5: To a stirred solution of 4-(pentafluoro-λ6-sulfanyl) benzoic acid (0.200 g, 0.000806 mol) in DMF (5.00 mL), was added [dimethylamino(triazolo[4,5-b]pyridin-3-yloxy)methylene]-dimethyl-ammonium;hexafluorophosphate (0.460 g, 0.00121 mol), cooled to 0° C., then N-ethyl-N-isopropyl-propan-2-amine (0.521 g, 0.00403 mol), stirred for 5 minutes, 3-[6-fluoro-7-(4-piperidyl)-3H-imidazo[4,5-b]pyridin-2-yl]cyclobutanol (0.281 g, 0.000967 mol). The reaction mixture was stirred at RT for 16 hours. The reaction mixture was monitored by TLC and LCMS. After completion (TLC and LC/MS), the reaction mixture was diluted with water (10 mL), extracted with ethyl acetate (2×10 mL), organic layers were dried over sodium sulphate and concentrated under vacuum to get crude which was purified by preparative HPLC (Sample code: EXP-23-LJ66-045-C Mobile Phase: A=0.1% HCOOH IN WATER B=ACN Column: Gemini NX (250 mm×21.2 mm), 5.0μ Flow: 18 mL/min Gradient Programmer: Time % B 0 20 2 30 8 60 PURITY>95%), pure fractions were concentrated to afford [4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone (0.210 g, 0.000403 mol, yield: 50.1%) as a white solid. LC/MS: t_{RA}=3.017 min, m/z: 521 [M−H]

Step 6: Chiral separation: For chiral separation, 210 mg submitted for SFC purification, (Chiral method: Column: CHIRALPAK-IC (150×4.6 mm×5 μm) Mobile Phase: (A) n-hexane (B) IPA:MeOH (50:50) Isocratic: 80:20 (A:B) Flow: 1.0 ml/min Diluent: EtOH Column Temp: 25° C.), After chiral separation, pure fractions were lyophilized to afford (cis)-(4-(6-fluoro-2-(3-hydroxycyclobutyl)-3H-imidazo[4,5-b]pyridin-7-yl)piperidin-1-yl)(4-(pentafluoro-λ⁶-sulfanyl) phenyl)methanone (0.127 g, 0.000244 mol, yield: 63.5%) LC/MS: t_{RA}=3.28 min, m/z: 521 [M+H]. HPLC: t_{RA}=5.46 min, 98.93%. ¹H NMR (400 MHz, DMSO-d₆) δ ppm 12.84-12.97 (m, 1H) 8.12-8.20 (m, 1H) 7.98-8.09 (m, 2H) 7.64-7.76 (m, 2H) 5.22-5.35 (m, 1H) 4.58-4.75 (m, 1H) 4.04-4.23 (m, 1H) 3.48-3.67 (m, 2H) 2.91-3.15 (m, 2H) 2.61-2.70 (m, 3H) 2.20-2.33 (m, 5H) 1.70-1.94 (m, 2H). Chiral HPLC: t_{RA}=6.65 min, 99.4% and (trans)-(4-(6-fluoro-2-(3-hydroxycyclobutyl)-3H-imidazo[4,5-b]pyridin-7-yl)piperidin-1-yl)(4-(pentafluoro-λ⁶-sulfanyl)phenyl)methanone (0.00850 g, 1.63e−5 mol, yield: 4.25%). LC-MS: t_{RA}=3.08 min, m/z: 521 [M+H]. HPLC: t_{RA}=4.25 min, 97.60%. Chiral HPLC: t_{RA}=7.61 min, 98.4%. ¹H NMR (400 MHz, DMSO-d₆) δ ppm 8.18-8.26 (m, 1H) 8.10-8.18 (m, 1H) 8.00-8.08 (m, 2H) 7.99-8.05 (m, 2H) 7.64-7.73 (m, 2H) 7.61-7.66 (m, 1H) 5.18-5.26 (m, 1H) 4.63-4.72 (m, 1H) 4.41-4.54 (m, 1H) 3.52-3.66 (m, 3H) 2.57-2.67 (m, 3H) 2.18-2.41 (m, 4H)

### Example 447: [2-amino-4-(trifluoromethoxy)phenyl]-[4-[6-fluoro-2-(1-methoxy-1-methyl-ethyl)-3H-imidazo[4,5-b]pyridin-7-yl]-1-piperidyl]methanone

Example 447 was prepared following a procedure similar to that of step 2 of Example 451 using 2-methoxy-2-methylpropanal, followed by a procedure similar to that of step 3 of Example 451. LC/MS (m/z, M+H, Method 2): 496; ¹H NMR (400 MHz, DMSO-d6, 100° C.) δ ppm 1.62 (s, 6H), 1.81 (br d, J=12.1 Hz, 2H), 2.26-2.42 (m, 2H), 3.01-3.19 (m, 2H), 3.10 (s, 3H), 3.50-3.69 (m, 1H), 4.17 (br d, J=12.1 Hz, 2H), 5.33 (br s, 2H), 6.49 (br d, J=8.4 Hz, 1H), 6.64-6.73 (m, 1H), 7.13 (d, J=8.4 Hz, 1H), 8.15 (br s, 1H), 12.44-12.95 (m, 1H)

### Example 449: 4-(6-fluoro-2-N-morpholino-3H-imidazo[4,5-b]pyridin-7-yl)-1-piperidyl]-[4-(pentafluoro-λ⁶-sulfanyl)phenyl]methanone

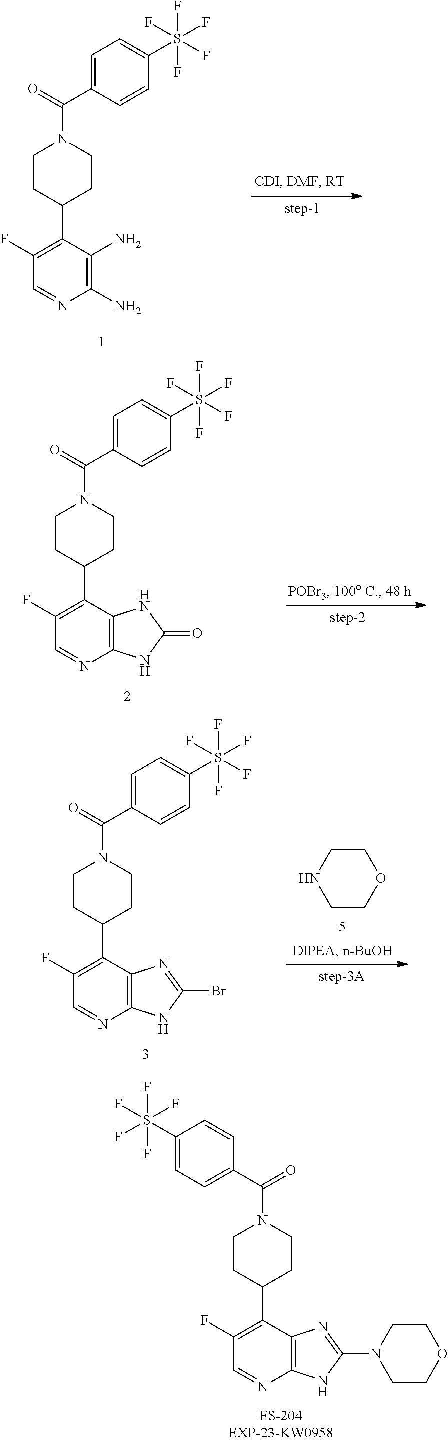 [SMILES: Nc1ncc(F)c(C2CCN(C(=O)c3ccc(S(F)(F)(F)(F)F)cc3)CC2)c1N ; O=C(c1ccc(S(F)(F)(F)(F)F)cc1)N1CCC(c2c(F)cnc3[nH]c(=O)[nH]c23)CC1 ; O=C(c1ccc(S(F)(F)(F)(F)F)cc1)N1CCC(c2c(F)cnc3[nH]c(Br)nc23)CC1 ; C1COCCN1 ; O=C(c1ccc(S(F)(F)(F)(F)F)cc1)N1CCC(c2c(F)cnc3[nH]c(N4CCOCC4)nc23)CC1]

Step 1: To a stirred solution of [4-(2,3-diamino-5-fluoro-4-pyridyl)-1-piperidyl]-[4-(pentafluoro-λ6-sulfanyl) phenyl]methanone (0.800 g, 0.00182 mol) in DMF (5.00 mL), were added di(imidazol-1-yl)methanone (1.47 g, 0.00908 mol) at RT and stirred for 16 hours at RT. The reaction progress was monitored by TLC and LCMS. After completion (TLC and LC/MS), the reaction mixture was quenched with water (10 mL), solid precipitated out was filtered, washed with hexane (10 mL) to get crude 6-fluoro-7-[1-[4-(pentafluoro-λ⁶-sulfanyl) benzoyl]-4-piperidyl]-1,3-dihydroimidazo[4,5-b]pyridin-2-one (80.0%, 0.500 g, 0.000858 mol, yield: 47.2%) as an off-white solid. LC/MS: t_{RA}=1.65 min, m/z: 467 [M+H].

Step 2: To a stirred solution of 6-fluoro-7-[1-[4-(pentafluoro-λ6-sulfanyl)benzoyl]-4-piperidyl]-1,3-dihydroimidazo[4,5-b]pyridin-2-one (0.400 g, 0.000858 mol) in POBr₃ (4.0 g), was heated to 110° C. and stirred for 16 hours at 110° C. The reaction progress was monitored by TLC and LCMS. After completion (TLC and LC/MS), the reaction mixture was quenched with saturated NaHCO₃ (10 mL), extracted with EtOAc (2×10 mL), organic layer were dried over sodium sulphate, filtered, then organic layer was concentrated under vacuum to afford crude [4-(2-bromo-6-fluoro-3H-imidazo[4,5-b]pyridin-7-yl)-1-piperidyl]-[4-(pentafluoro-λ6-sulfanyl)phenyl]methanone (20.0%, 0.300 g, 0.000113 mol, yield: 13.2%) as brown solid. LC/MS: t_{RA}=1.828 min, m/z: 531 [M+2].

Step 3: To a stirred solution of [4-(2-bromo-6-fluoro-3H-imidazo[4,5-b]pyridin-7-yl)-1-piperidyl]-[4-(pentafluoro-λ6-sulfanyl)phenyl]methanone (0.200 g, 0.000378 mol) in n-butanol (5.00 mL), were added N-ethyl-N-isopropyl-propan-2-amine (0.147 g, 0.00113 mol) and morpholine (0.0988 g, 0.00113 mol) at RT and heated to 100° C. for 16 hours. The reaction progress was monitored by TLC and LCMS. After completion (TLC and LC/MS), the reaction mixture was concentrated under vacuum, diluted with water (10 mL), extracted with ethyl acetate (2×10 mL), Organic layer were dried over sodium sulphate and concentrated under vacuum to get crude which was purified by preparative HPLC, (Mobile Phase: A=0.1% HCOOH IN WATER B=ACN Column: X SELECT (250 mm×20.0 mm), 5.0μ Flow: 15 ml/min Gradient Programmer: Time % B 0 10 2 20 8 50 PURITY >95%:) pure fractions were concentrated to afford [4-(6-fluoro-2-morpholino-3H-imidazo[4,5-b]pyridin-7-yl)-1-piperidyl]-[4-(pentafluoro-λ4⁶-sulfanyl)phenyl]methanone (0.0124 g, 2.32e−5 mol, yield: 6.13%) as an off-white solid. LC/MS: t_{RA}=1.763 min, m/z: 536 [M+H]. ¹H NMR (400 MHz, DMSO-d₆) δ ppm 12.04-12.20 (m, 1H) 7.94-8.09 (m, 2H) 7.74-7.89 (m, 1H) 7.54-7.70 (m, 2H) 4.54-4.77 (m, 1H) 3.67-3.83 (m, 4H) 3.48-3.61 (m, 6H) 2.85-3.00 (m, 1H) 2.16-2.37 (m, 2H) 1.57-1.90 (m, 2H).

### Example 450: (trans)-[4-[6-fluoro-2-[4-hydroxy-4-(trifluoromethyl)cyclohexyl]-3H-imidazo[4,5-b]pyridin-7-yl]-1-piperidyl]-[4-(pentafluoro-λ⁶-sulfanyl)phenyl]methanone

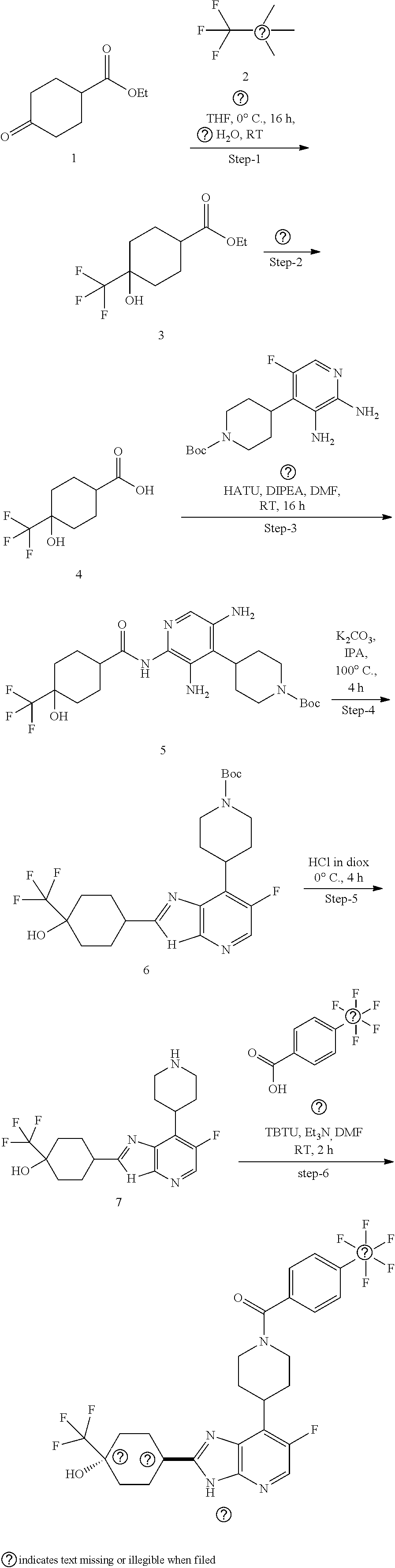

Step 1: To a stirred solution of ethyl 4-oxocyclohexanecarboxylate (5.00 g, 0.0294 mol) and cesium fluoride (5.35 g, 0.0353 mol) in 1,2-dimethoxyethane (50.0 mL), trimethyl(trifluoromethyl)silane (5.01 g, 0.0353 mol) was added drop wise at 0′° C. Reaction mixture was warmed to RT and stirred at ambient temperature for 16 hours. After 16 hours, tetrabutylammonium fluoride (7.68 g, 0.0294 mol) was added drop wise at 0° C., stirred for 30 min. The reaction was monitored by TLC and LCMS. After completion (TLC and LC/MS), the volatiles were evaporated and the residue was quenched with ice water (50 mL), extracted with ethyl acetate (3×50 mL). All the organics were washed with water (2×20 mL), saturated brine solution (20 mL) and the organic layer was dried over Na₂SO₄, filtered off and concentrated to afford crude compound as brown oil which was purified by column chromatography using hexane/EtOAc as an eluent. Product eluted at 20% EtOAc in hexane were concentrated to obtain ethyl 4-hydroxy-4-(trifluoromethyl) cyclohexane carboxylate (3.50 g, 0.0146 mol, yield: 49.6%) as a colorless oil. LC/MS: t_{RA}=2.786 min, m/z: 239.2 [M−H]

Step 2: To a stirred solution of ethyl 4-hydroxy-4-(trifluoromethyl) cyclohexane carboxylate (3.50 g, 0.0146 mol) in 1,4-dioxane (35.0 mL), NaOH (0.699 g, 0.0175 mol) in water (3.5 ml) was added drop wise at 0° C. The reaction mixture was warmed to RT and stirred at 80° C. for 2 hours. The reaction was monitored by TLC and LCMS. After completion (TLC and LC/MS), volatiles were evaporated and the residue was quenched with ice water (20 mL), extracted with diethyl ether (3×25 mL). The aqueous layer was acidified with 4N HCl, the precipitated solids were filtered and dried to afford 4-hydroxy-4-(trifluoromethyl) cyclohexane carboxylic acid (2.00 g, 0.00943 mol, yield: 64.7%) as an off-white solid. LC/MS: t_{RA}=1.70 min, m/z: 211.1 [M−H]

Step 3: To a stirred solution of 4-hydroxy-4-(trifluoromethyl)cyclohexane carboxylic acid (1.00 g, 0.00471 mol), tert-butyl 4-(2,3-diamino-5-fluoro-4-pyridyl)piperidine-1-carboxylate (1.46 g, 0.00471 mol), N-ethyl-N-isopropyl-propan-2-amine (0.609 g, 0.00471 mol) and 2,4,6-tripropyl-1,3,5,2λ5,4λ5,6lλ5-trioxatriphosphinane 2,4,6-trioxide (2.25 g, 0.00707 mol) in N,N-dimethylformamide (10.0 mL) at 0° C. Reaction mixture was warmed to RT and stirred at ambient temperature for 16 hours. The reaction was monitored by TLC and LCMS. After completion (TLC and LC/MS), the reaction mixture was quenched with ice water (20 mL), the precipitated solids were filtered and dried to afford tert-butyl 4-[3-amino-5-fluoro-2-[[4-hydroxy-4-(trifluoromethyl)cyclohexanecarbonyl]amino]-4-pyridyl]piperidine-1-carboxylate (1.60 g, 0.00317 mol, yield: 67.3%) used for next step without further purifications. LC/MS: t_{RA}=2.608 min, m/z: 503.2 [M−H]

Step 4: To a stirred solution of tert-butyl 4-[2-amino-5-fluoro-3-[[4-hydroxy-4-(trifluoromethyl)cyclohexanecarbonyl]amino]-4-pyridyl]piperidine-1-carboxylate (1.80 g, 0.00357 mol) and dipotassium; carbonate (1.48 g, 0.0107 mol) in propan-2-ol (15.0 mL). The reaction mixture was stirred at 100° C. for 4 hours. The reaction was monitored by TLC and LCMS. After completion (TLC and LC/MS), volatiles were evaporated and the residue was quenched with ice water (10 mL), extracted with EtOAc (3×25 mL). Organics were washed with water (2×10 mL), sat. brine solution (10 mL) and the organic layer was dried over Na₂SO₄, filtered and concentrated to afford crude compound as brown solid. The crude material was triturated with diethyl ether to obtain tert-butyl 4-[6-fluoro-2-[4-hydroxy-4-(trifluoromethyl)cyclohexyl]-3H-imidazo[4,5-b]pyridin-7-yl]piperidine-1-carboxylate (1.20 g, 0.00247 mol, yield: 69.1%) as an off white solid. LC/MS: t_{RA}=2.08 min, m/z: 487.15 [M+H]

Step 5: To a stirred solution of tert-butyl 4-[6-fluoro-2-[4-hydroxy-4-(trifluoromethyl)cyclohexyl]-3H-imidazo[4,5-b]pyridin-7-yl]piperidine-1-carboxylate (0.403 g, 0.000828 mol) in 1,4-dioxane (10.0 mL), hydrogen chloride (0.0604 g, 0.00166 mol) was added drop wise at 0° C. Reaction mixture was warmed to RT and stirred at RT for 2 hours. The reaction was monitored by TLC and LCMS. After completion (TLC and LC/MS), volatiles were evaporated and the residue was triturated with diethyl ether to afford 4-[6-fluoro-7-(4-piperidyl)-3H-imidazo[4,5-b]pyridin-2-yl]-1-(trifluoromethyl)cyclohexanol (0.700 g, 0.00181 mol, yield: 73.4%) as a brown solid. LC/MS: t_{RA}=1.98 min, m/z: 387.10 [M+H]

Step 6: To a stirred solution of 4-(pentafluoro-λ6-sulfanyl)benzoic acid (0.247 g, 0.000994 mol) in N,N-dimethylformamide (6.0 mL), were added [benzotriazol-1-yloxy(dimethylamino)methylene]-dimethyl-ammonium;tetrafluoroborate (0.399 g, 0.00124 mmol) and N,N-diethylethanamine (0.251 g, 0.00248 mol) at 0° C. and continued for 15 minutes, then added 4-[6-fluoro-7-(4-piperidyl)-3H-imidazo[4,5-b]pyridin-2-yl]-1-(trifluoromethyl) cyclohexanol (0.320 g 0.000828 mmol) at 0° C. Reaction mixture was warmed to RT and stirred at RT for 16 hours. The reaction was monitored by TLC and LCMS. After completion (TLC and LC/MS), volatiles were evaporated and the residue was quenched with ice water (30 mL), extracted with EtOAc (3×30 mL). Organics were washed with water (2×30 mL), saturated brine solution (20 mL) and the organic layer was dried over Na₂SO₄, filtered and concentrated to afford crude compound which was purified by preparative HPLC (Mobile phase A: 0.1% TFA in water mobile phase B: Acetonitrile COLUMN: ZODIAC-C18 (19*250 mm) 5μ flow rate: 17 ml/min solubility: ACN+THF GRADIENT: TIME % B 0 30 2 40 8 70); pure fractions were lyophilized to afford (trans)-(4-(6-fluoro-2-(4-hydroxy-4-(trifluoromethyl)cyclohexyl)-3H-imidazo[4,5-b]pyridine-7-yl)piperidin-1-yl)(4-(pentafluoro-λ⁶-sulfaneyl)phenyl)methanone (14 mg, 0.00002 mol, yield 2.74%) as an off white solid. LC-MS: t_{RA}=1.83 min, m/z: 617.15 [M+H]; HPLC: t_{RA}=4.01 min, 90.02%; ¹H NMR (400 MHz, DMSO-d₆) δ ppm 8.15-8.27 (m, 1H) 7.92-8.07 (m, 2H) 7.59-7.76 (m, 2H) 5.73-5.98 (m, 1H) 4.60-4.79 (m, 1H) 3.22-3.41 (m, 2H) 2.83-3.06 (m, 3H) 1.78-2.07 (m, 8H) 1.54-1.78 (m, 3H). ¹⁹F NMR (272 MHz, DMSO-d₆) δ=−68.90 (s, 1F), −71.41 (s, 1F), −74.50 (s, 1F), −82.86 (s, 1F), −185.43 (m, 1F). Chiral HPLC: t_{RA}=7.50 min, 94.62% (ee) (Method Column; REGIS(S,S) WHELK-01(150×4.6 mm×5 μm) Mobile Phase: (A) n-hexane (B) 0.1% NH₄OH in EtOH:IPA:MeOH (1:1:1) Isocratic 70:30 (A:B) Flow: 1.0 ml/min).

### Example 451 [2-Amino-4-(trifluoromethoxy)phenyl]-[4-[6-fluoro-2-(2-oxabicyclo[2.2.2]octan-4-yl)-3H-imidazo[4,5-b]pyridin-7-yl]-1-piperidyl]methanone

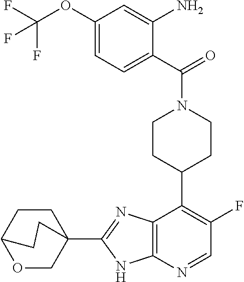 [SMILES: Nc1cc(OC(F)(F)F)ccc1C(=O)N1CCC(c2c(F)cnc3[nH]c(C45CCC(CC4)OC5)nc23)CC1]

### Step 1: tert-Butyl N-[2-[4-(2,3-diamino-5-fluoro-4-pyridyl)piperidine-1-carbonyl]-5-(trifluoromethoxy)phenyl]carbamate

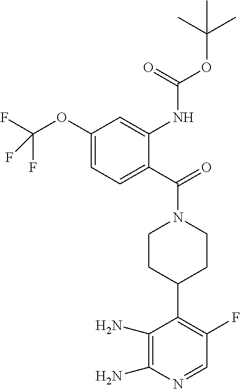 [SMILES: CC(C)(C)OC(=O)Nc1cc(OC(F)(F)F)ccc1C(=O)N1CCC(c2c(F)cnc(N)c2N)CC1]

To a solution of 5-fluoro-4-(4-piperidyl)pyridine-2,3-diamine;hydrochloride (338 mg, 1.19 mmol) in DMF (5 mL) wad added DIPEA (617 mg, 0.83 mL, 4.77 mmol). Then, 2-(tert-butoxycarbonylamino)-4-(trifluoromethoxy)benzoic acid (383 mg, 1.19 mmol) and TBTU (421 mg, 1.31 mmol) were added and the resulting mixture was stirred at room temperature for 1 hour. After one hour, the reaction mixture was diluted with ethyl acetate, washed with an aqueous saturated solution of NaHCO₃, water and brine. The organic layer was then dried over sodium sulfate, filtered and concentrated in vacuo. The resulting residue was purified on silica gel (SiO₂ 24 g) eluting with DCM/MeOH/NH₄OH 94/6/0.6 to give 508 mg (82% yield) of tert-butyl N-[2-[4-(2,3-diamino-5-fluoro-4-pyridyl)piperidine-1-carbonyl]-5-(trifluoromethoxy)phenyl]carbamate.

### Step 2: tert-Butyl N-[2-[4-[6-fluoro-2-(2-oxabicyclo[2.2.2]octan-4-yl)-3H-imidazo[4,5-b]pyridin-7-yl]piperidine-1-carbonyl]-5-(trifluoromethoxy)phenyl]carbamate

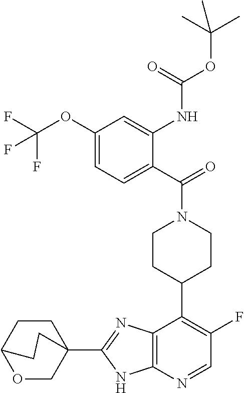 [SMILES: CC(C)(C)OC(=O)Nc1cc(OC(F)(F)F)ccc1C(=O)N1CCC(c2c(F)cnc3[nH]c(C45CCC(CC4)OC5)nc23)CC1]

In a microwave vial, were successively introduced tert-butyl N-[2-[4-(2,3-diamino-5-fluoro-4-pyridyl)piperidine-1-carbonyl]-5-(trifluoromethoxy)phenyl]carbamate (75 mg, 0.15 mmol), 2-oxabicyclo[2.2.2]octane-4-carbaldehyde (23 mg, 0.16 mmol) and DMF (2 mL). To this solution, was then added sodium metabisulfite (36 mg, 0.19 mmol). The vial was then sealed and submitted to microwave at 125° C. for 90 minutes. After 90 minutes, the vial was cooled down to room temperature, opened, diluted with AcOEt, transferred to a separating funnel, and extracted twice with AcOEt. The combined organic layers were then washed with water, dried over magnesium sulfate, filtered, concentrated in vacuo. The resulting residue was purified by flash chromatography to give 92 mg (100% yield) of tert-butyl N-[2-[4-[6-fluoro-2-(2-oxabicyclo[2.2.2]octan-4-yl)-3H-imidazo[4,5-b]pyridin-7-yl]piperidine-1-carbonyl]-5-(trifluoromethoxy)phenyl]carbamate as a pale yellow wax.

### Step 3: [2-Amino-4-(trifluoromethoxy)phenyl]-[4-[6-fluoro-2-(2-oxabicyclo[2.2.2]octan-4-yl)-3H-imidazo[4,5-b]pyridin-7-yl]-1-piperidyl]methanone

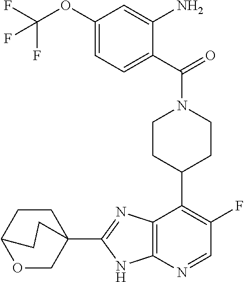 [SMILES: Nc1cc(OC(F)(F)F)ccc1C(=O)N1CCC(c2c(F)cnc3[nH]c(C45CCC(CC4)OC5)nc23)CC1]

To a solution of N-[2-[4-[6-fluoro-2-(2-oxabicyclo[2.2.2]octan-4-yl)-3H-imidazo[4,5-b]pyridin-7-yl]piperidine-1-carbonyl]-5-(trifluoromethoxy)phenyl]carbamate (92 mg, 0.14 mmol) in MeOH (1 mL) was added 800 μL of a 4N HCl solution in dioxane. The resulting reaction mixture was stirred for 16 hours at room temperature. After 16 hours, the reaction mixture was concentrated in vacuo and the resulting residue was purified on silica gel (SiO₂ 12 g) eluting with DCM/iPrOH 90/10 to give 12 mg (7% yield) of 2-amino-4-(trifluoromethoxy)phenyl]-[4-[6-fluoro-2-(2-oxabicyclo[2.2.2]octan-4-yl)-3H-imidazo[4,5-b]pyridin-7-yl]-1-piperidyl]methanone. LC/MS (m/z, M+H, Method 1): calc. 534.2, found 534.4; ¹H NMR (400 MHz, DMSO-d6, 100° C.) δ ppm 1.67-1.85 (m, 4H), 2.00-2.20 (m, 6H), 2.23-2.41 (m, 2H), 3.08 (br t, J=12.1 Hz, 2H), 3.45-3.64 (m, 1H), 3.76-3.85 (m, 1H), 4.03 (s, 2H), 4.17 (br d, J=12.1 Hz, 2H), 5.33 (br s, 2H), 6.49 (br d, J=8.4 Hz, 1H), 6.70 (br s, 1H), 7.14 (d, J=8.4 Hz, 1H), 8.11 (br d, J=3.0 Hz, 1H), 12.14-12.95 (m, 1H)

### Examples 452 and 455: (rac)-[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone, and (rac)-2-[6-fluoro-7-[1-[4-(pentafluoro-λ⁶-sulfanyl)benzoyl]-4-piperidyl]-3H-imidazo[4,5-b]pyridin-2-yl]-2,3,4a,5,7,7a-hexahydrofuro [3,4-b][1,4]oxazine-4-carboxylic acid

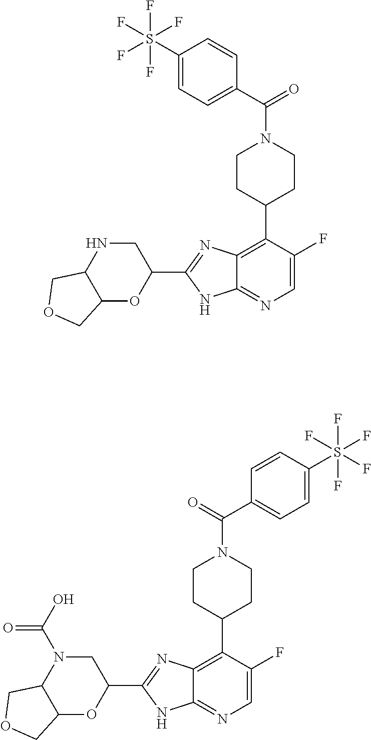 [SMILES: O=C(c1ccc(S(F)(F)(F)(F)F)cc1)N1CCC(c2c(F)cnc3[nH]c(C4CNC5COCC5O4)nc23)CC1 ; O=C(c1ccc(S(F)(F)(F)(F)F)cc1)N1CCC(c2c(F)cnc3[nH]c(C4CN(C(=O)O)C5COCC5O4)nc23)CC1]

### Step 1: tert-butyl 2-[[2-amino-5-fluoro-4-[1-[4-(pentafluoro-λ⁶-sulfanyl)benzoyl]-4-piperidyl]-3-pyridyl]carbamoyl]-2,3,4a,5,7,7a-hexahydrofuro[3,4-b][1,4]oxazine-4-carboxylate

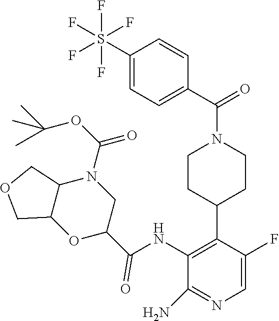 [SMILES: CC(C)(C)OC(=O)N1CC(C(=O)Nc2c(N)ncc(F)c2C2CCN(C(=O)c3ccc(S(F)(F)(F)(F)F)cc3)CC2)OC2COCC21]

Step 1 was performed following the protocol described in step 2 of Example 364 using [4-(2,3-diamino-5-fluoro-4-pyridyl)-1-piperidyl]-[4-(pentafluoro-λ6-sulfanyl)phenyl]methanone (described in step 1 of Example 364) and 4-[(tert-butoxy)carbonyl]-hexahydro-2H-furo[3,4-B]morpholine-2-carboxylic acid, to give 110 mg (56% yield) of tert-butyl 2-[[2-amino-5-fluoro-4-[1-[4-(pentafluoro-λ6-sulfanyl)benzoyl]-4-piperidyl]-3-pyridyl]carbamoyl]-2,3,4a,5,7,7a-hexahydrofuro[3,4-b][1,4]oxazine-4-carboxylate as a brown solid.

### Step 2: tert-butyl 2-[6-fluoro-7-[1-[4-(pentafluoro-λ6-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylate

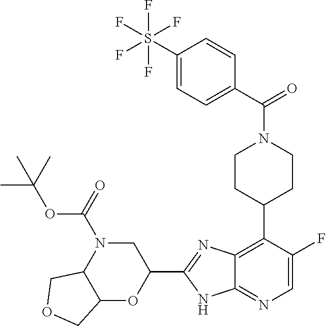 [SMILES: CC(C)(C)OC(=O)N1CC(c2nc3c(C4CCN(C(=O)c5ccc(S(F)(F)(F)(F)F)cc5)CC4)c(F)cnc3[nH]2)OC2COCC21]

In a 2-5 mL microwave vial, to a solution of tert-butyl 2-[[2-amino-5-fluoro-4-[1-[4-(pentafluoro-λ6-sulfanyl)benzoyl]-4-piperidyl]-3-pyridyl]carbamoyl]-2,3,4a,5,7,7a-hexahydrofuro[3,4-b][1,4]oxazine-4-carboxylate (110 mg, 0.08 mmol) in i-PrOH (3 mL) was added K₂CO₃ (22 mg, 0.16 mmol). The vial was sealed and submitted to microwave at 110° C. for one hour. After one hour, the reaction mixture was concentrated in vacuo, diluted with DCM and water, transferred to a separating funnel, and extracted three times with DCM. The combined organic layers were washed with water, brine, dried over magnesium sulfate, filtered and concentrated. The resulting residue was purified on silica gel (SiO₂ 24 g) eluting with DCM/i-PrOH 100/0 to 90/10 to give 48 mg (42% yield) of tert-butyl 2-[6-fluoro-7-[1-[4-(pentafluoro-λ6-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylate as white solid.

### Step 3: (rac)-[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone, and (rac)-2-[6-fluoro-7-[1-[4-(pentafluoro-λ6-sulfanyl)benzoyl]-4-piperidyl]-3H-imidazo[4,5-b]pyridin-2-yl]-2,3,4a,5,7,7a-hexahydrofuro [3,4-b][1,4]oxazine-4-carboxylic acid

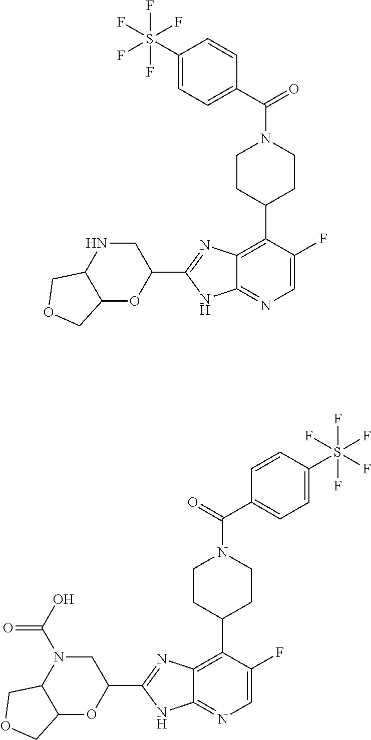 [SMILES: O=C(c1ccc(S(F)(F)(F)(F)F)cc1)N1CCC(c2c(F)cnc3[nH]c(C4CNC5COCC5O4)nc23)CC1 ; O=C(c1ccc(S(F)(F)(F)(F)F)cc1)N1CCC(c2c(F)cnc3[nH]c(C4CN(C(=O)O)C5COCC5O4)nc23)CC1]

To a solution of tert-butyl 2-[6-fluoro-7-[1-[4-(pentafluoro-λ6-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylate (65 mg, 0.096 mmol) in DCM (5 mL), was added 0.19 mL of a 4M HCl solution in dioxane (0.19 mL, 0.78 mmol) and the resulting reaction mixture was stirred for 48 hours at room temperature. Then, the reaction mixture was concentrated in vacuo, diluted with AcOEt and water, transferred to separating funnel containing an aqueous saturated solution of NaHCO₃, and extracted twice with AcOEt. The combined organic layers were then washed with water, brine, dried over magnesium sulfate, filtered and concentrated. The resulting residue was purified on silica gel (SiO₂ 12 g) eluting with DCM/i-PrOH 100/0 to 90/10 to give 12 mg (18% yield) of (rac)-2-[6-fluoro-7-[1-[4-(pentafluoro-λ6-sulfanyl)benzoyl]-4-piperidyl]-3H-imidazo[4,5-b]pyridin-2-yl]-2,3,4a,5,7,7a-hexahydrofuro [3,4-b][1,4]oxazine-4-carboxylic acid as a white solid, and 10 mg (17% yield) of (rac)-[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone as white solid.

Example 452: LC/MS (m/z, M+H, Method 1): calc. 578.5, found 578.4

Example 455: LC/MS (m/z, M+H, Method 1): calc. 622.1, found 622.4

### Examples 453 and 454: (trans)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone and (cis)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone

Examples 453 and 454 were obtained by isomeric separation of the compound of Example 391 using Method CS14.

Example 453 (trans-isomer): LC/MS (m/z, M+H, Method 2): 550; ¹H NMR (400 MHz, DMSO-d6, 100° C.) δ ppm 1.82 (br d, J=11.4 Hz, 2H), 2.35-2.43 (m, 4H), 2.59-2.69 (m, 2H), 3.10 (t, J=12.5 Hz, 2H), 3.20 (s, 3H), 3.48-3.60 (m, 1H), 3.61-3.70 (m, 1H), 4.06-4.25 (m, 3H), 5.45 (br s, 2H), 7.01 (dd, J=8.4, 2.3 Hz, 1H), 7.23 (br d, J=8.3 Hz, 1H), 7.28 (d, J=2.3 Hz, 1H), 8.10 (br s, 1H), 12.42-12.72 (m, 1H)

Example 454 (cis-isomer): LC/MS (m/z, M+H, Method 2): 550; ¹H NMR (400 MHz, DMSO-d6, 100° C.) δ ppm 1.77-1.86 (m, 2H), 2.24-2.37 (m, 4H), 2.61-2.71 (m, 2H), 3.05-3.16 (m, 2H), 3.21 (s, 3H), 3.21-3.28 (m, 1H), 3.49-3.63 (m, 1H), 3.88-3.97 (m, 1H), 4.09-4.22 (m, 2H), 5.44 (br s, 2H), 7.02 (dd, J=8.4, 2.3 Hz, 1H), 7.24 (d, J=8.4 Hz, 1H), 7.28 (d, J=2.3 Hz, 1H), 8.10 (br s, 1H), 12.37-12.77 (m, 1H)

### Example 456: (rac)-[4-[2-(1,1-dioxo-1,4-thiazinan-2-yl)-6-fluoro-3H-imidazo[4,5-b]pyridin-7-yl]-1-piperidyl]-[4-(pentafluoro-λ⁶-sulfanyl)phenyl]methanone

Example 456 was prepared by a procedure similar to that of step 2 of Example 364 using 4-tert-butoxycarbonyl-1,1-dioxo-1,4-thiazinane-2-carboxylic acid to give tert-butyl 2-[[2-amino-5-fluoro-4-[1-[4-(pentafluoro-λ⁶-sulfanyl)benzoyl]-4-piperidyl]-3-pyridyl]carbamoyl]-1,1-dioxo-1,4-thiazinane-4-carboxylate, followed by a procedure similar to that of step 7 of Examples 355 and 356.

### Example 457: [4-[6-fluoro-2-(3-methoxycyclobutyl)-3H-imidazo[4,5-b]pyridin-7-yl]-1-piperidyl]-[4-(trifluoromethoxy)phenyl]methanone

Example 457 was prepared by a procedure similar to that of Example 429 using 3-methoxycyclobutane-1-carbaldehyde in step 3. LC/MS (m/z, M+H, Method 2): 493; ¹H NMR (400 MHz, DMSO-d6, 100° C., mixture of diastereoisomers 90/10) δ ppm 1.81 (br d, J=13.1 Hz, 2H), 2.17-2.44 (m, 4H), 2.59-2.73 (m, 2H), 3.04-3.16 (m, 2H), 3.16-3.30 (m, 4H), 3.49-3.61 (m, 0.9H), 3.61-3.72 (m, 0.1H), 3.87-3.98 (m, 0.9H), 4.10-4.29 (m, 2.10H), 7.39 (br d, J=7.9 Hz, 2H), 7.53-7.59 (m, 2H), 8.10 (d, J=3.4 Hz, 1H), 12.16-13.01 (m, 1H)

### Example 458: (2-amino-4-(trifluoromethoxy) phenyl]-[4-[6-fluoro-2-(3-methoxycyclobutyl)-3Himidazo[4,5-b]pyridin-7-yl]-1-piperidyl]methanone

Example 458 was prepared by a procedure similar to that of Example 451 using 3-methoxycyclobutane-1-carbaldehyde in step 2. LC/MS (m/z, M+H, Method 3): 508; ¹H NMR (400 MHz, DMSO-d6 and acetic acid-d4, 100° C., mixture of diastereoisomers 70/30) δ ppm 1.81 (br d, J=12.6 Hz, 2H), 2.19-2.44 (m, 4H), 2.60-2.72 (m, 2H), 3.02-3.15 (m, 2H), 3.17-3.31 (m, 4H), 3.55 (ddd, J=12.1, 8.5, 4.1 Hz, 0.7H), 3.67 (tt, J=9.6, 4.5 Hz, 0.3H), 3.87-4.02 (m, 0.7H), 4.10-4.28 (m, 2.30H), 6.46-6.54 (m, 1H), 6.70 (br s, 1H), 7.11-7.21 (m, 1H), 8.06-8.14 (m, 1H)

### Examples 459 and 460: (cis)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone and (trans)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone

Examples 459 and 460 were prepared by a procedure similar to that of Example 451 using 4-methoxycyclohexanecarbaldehyde in step 2.

Example 459 (cis-isomer): LC/MS (m/z, M+H, Method 2): 536; ¹H NMR (400 MHz, DMSO-d6, 100° C.) δ ppm 1.54-1.65 (m, 2H), 1.74-1.84 (m, 4H), 1.85-1.94 (m, 2H), 1.94-2.07 (m, 2H), 2.19-2.38 (m, 2H), 2.88-3.02 (m hidden, 1H), 3.08 (td, J=12.8, 2.0 Hz, 2H), 3.26 (s, 3H), 3.38-3.46 (m, 1H), 3.47-3.62 (m, 1H), 4.17 (br d, J=12.8 Hz, 2H), 5.33 (s, 2H), 6.49 (br d, J=8.4 Hz, 1H), 6.65-6.73 (m, 1H), 7.15 (d, J=8.4 Hz, 1H), 8.08 (d, J=3.3 Hz, 1H), 12.11-12.73 (m, 1H)

Example 460 (trans-isomer): LC/MS (m/z, M+H, Method 2): 536.5; ¹H NMR (400 MHz, DMSO-d6, 100° C.) δ ppm 1.23-1.39 (m, 2H), 1.60-1.75 (m, 2H), 1.80 (br d, J=12.5 Hz, 2H), 2.11 (br d, J=10.8 Hz, 4H), 2.19-2.36 (m, 2H), 2.84 (tt, J=11.5, 3.3 Hz, 1H), 3.01-3.12 (m, 2H), 3.21 (tt, J=10.3, 3.7 Hz, 1H), 3.28 (s, 3H), 3.46-3.58 (m, 1H), 4.17 (br d, J=12.1 Hz, 2H), 5.34 (s, 2H), 6.49 (dd, J=8.3, 1.1 Hz, 1H), 6.66-6.74 (m, 1H), 7.14 (d, J=8.4 Hz, 1H), 8.09 (d, J=3.4 Hz, 1H), 12.11-12.69 (m, 1H)

### Example 461: (rac)-7-[6-fluoro-7-[1-[4-(trifluoromethoxy)benzoyl]-4-piperidyl]-3H-imidazo[4,5-b]pyridin-2-yl]-2-oxa-5-azaspiro[3.5]nonane-5-carboxylic acid

Example 461 was prepared by a procedure similar to that of step 2 of Example 364 using sodium;5-tert-butoxycarbonyl-2-oxa-5-azaspiro[3.5]nonane-7-carboxylate and [4-(2,3-diamino-5-fluoro-4-pyridyl)-1-piperidyl]-[4-(trifluoromethoxy)phenyl]methanone (described in step 2 of Example 429), followed by a procedure similar to Example 378.

### Example 462—ERK5 Inhibitory Activity of Compounds

Two assays were performed to assess the ERK5 inhibitory activity of compounds of the Examples, a cell-based assay and a cell-free biochemical assay. Results of the assays are shown below in Table 2.

### Inhibition of Cellular ERK5 Activity

The renal cancer cell line SN12C was transduced by a lentivirus pGreenFire1 MEF2 EF1 Neo (ref TR030VA-N) from SBI using standard infection protocol. pGreenFire1 MEF2 EF1 Neo allows the expression of luciferase gene under the control of minimal promoter with MEF2 transcriptional response elements. Cells harboring the reporter construct were selected by geneticin treatment. The selected cells were then transposed by a piggyback based plasmid pCM4007 allowing the expression of constitutively activated MEK5DD under control of TREG3 promoter, a doxycycline regulated promoter. Transposed cells were selected by puromycin treatment. Upon doxycycline treatment (1 μg/ml) MEK5DD was expressed. MEK5DD activates ERK5 that phosphorylates MEF2C protein. Activated MEF2C protein can bind to its transcriptional response elements. Then the luciferase is expressed. In a 96-well plate (96F nuncleon ref137101 thermofisher), 50,000 cells were inoculated in 142.5 μl of RPMI medium containing 10% fetal calf serum, 1% glutamine, and 1 μg/ml doxycycline. After 24 hours, compounds were added in 7.5 μl culture medium (with 2% DMSO to give a final concentration of 0.1%) in order to obtain the desired concentration (0.3-10000 nM). The luciferase activity was measured using the Kit Bright Glo Luminescent Cell Assay Cat E2610 (Promega) according to the manufacturer's protocol. Luminescence was determined using 0.2 second reading/well using a Tecan SPARK. IC₅₀ values were calculated using XLFIT5 for Microsoft Excel using method 205. The IC₅₀ values represent the concentration of compound which inhibits the measurable luminescence signal by 50% as compared to DMSO-treated control cells.

### Cell-Free Assay of ERK5 Inhibition

An assay was performed to measure the capacity of each compound to inhibit ERK5 enzymatic activity. Compound potency was evaluated by Time-Resolved Forster Resonance Energy Transfer (FRET system). The activated catalytic domain of protein ERK5 (CarnaBiosciences #04-146) was mixed at 4 nM with varying concentrations of compound and incubated for 30 minutes at room temperature. A mixture of 1 mM ATP and 1 μM biotinylated synthetic peptide was added (Biosyntan GmbH). This synthetic peptide represents amino acids 30-52 of Eukaryotic translation initiation factor 4E-binding protein 1 (see, e.g., the sequence under accession No. NP_004086.1) biotinylated at the N-terminus. After 30 minutes at 37° C., the peptide phosphorylation by ERK5 was measured by addition of FRET reagents consisting of 12.5 μg/ml Streptavidin-XL665, 1 nM Anti-P-4EBP1 antibody and 300 ng/ml Anti-rabbit-K antibody. Following 90 minutes at room temperature fluorescent signals were read on the Pherastar FSX multimod detector from BMG Labtech (Exc° 340 nm, Em1 620 nm, Em2 665 nm). The IC₅₀ values represent the concentration of compound which inhibits the measurable fluorescence signal by 50% as compared to the DMSO only control.

TABLE 2
Results of cell-based and cell-free assays

| Compound | Cell-based assay | Cell-free assay |
|---|---|---|
| (Example No.) | (IC₅₀ in nM) | (IC₅₀ in nM) |
| 1 | 95 | 108 |
| 2 | 134 | 140 |
| 3 | 35 | 21 |
| 4 | 35 | 38 |
| 5 | 26 | 22 |
| 6 | 420 | 77 |
| 7 | 57 | 54 |
| 8 | 87 | 58 |
| 9 | 47 | 37 |
| 10 | 3582 | 2737 |
| 11 | 37 | 65 |
| 12 | 31 | 47 |
| 13 | 75 | 44 |
| 14 | 1025 | 2813 |
| 15 | 23 | 64 |
| 16 | 341 | 56 |
| 17 | 42 | 366 |
| 18 | 765 | 1200 |
| 19 | 115 | 386 |
| 20 | 122 | 213 |
| 21 | 2441 | 637 |
| 22 | 32 | 24 |
| 23 | 211 | 185 |
| 24 | 150 | 148 |
| 25 | 179 | 148 |
| 26 | 79 | 140 |
| 27 | 98 | 151 |
| 28 | 83 | 110 |
| 29 | 23 | 11 |
| 30 | 279 | 377 |
| 31 | 229 | 324 |
| 32 | 240 | 353 |
| 33 | 328 | 678 |
| 34 | 86 | 102 |
| 35 | 2337 | 1700 |
| 36 | 143 | 136 |
| 37 | 47 | 47 |
| 38 | 416 | 845 |
| 39 | 236 | 302 |
| 40 | 664 | 1460 |
| 41 | 644 | 2250 |
| 42 | 192 | 424 |
| 43 | 115 | 147 |
| 44 | 52 | 40 |
| 45 | 311 | 519 |
| 46 | 149 | 207 |
| 47 | 111 | 157 |
| 48 | 3046 | 4490 |
| 49 | 1178 | 2170 |
| 50 | 189 | 145 |
| 51 | 158 | 89 |
| 52 | 2955 | 15300 |
| 53 | >10000 | 262 |
| 54 | 534 | 776 |
| 55 | 1998 | 3520 |
| 56 | 216 | 288 |
| 57 | 57 | 85 |
| 58 | >10000 | — |
| 59 | 5294 | 23360 |
| 60 | 471 | 1039 |
| 61 | 291 | 495 |
| 62 | 627 | 492 |
| 63 | 368 | 74 |
| 64 | 869 | 1017 |
| 65 | 97 | 46 |
| 66 | 914 | 2345 |
| 67 | 163 | 181 |
| 68 | 146 | 198 |
| 69 | 122 | 367 |
| 70 | 57 | 90 |
| 71 | 383 | 963 |
| 72 | 83 | 25 |
| 73 | 200 | 60 |
| 74 | 305 | 74 |
| 75 | 25 | 73 |
| 76 | 77 | 165 |
| 77 | 166 | 648 |
| 78 | 3603 | — |
| 79 | 7890 | — |
| 80 | 6609 | 5410 |
| 81 | 296 | 259 |
| 82 | 272 | 361 |
| 83 | 3929 | 3020 |
| 84 | >10000 | 26800 |
| 85 | >10000 | — |
| 86 | >10000 | 25200 |
| 87 | >10000 | — |
| 88 | 7898 | 15600 |
| 89 | 3160 | — |
| 90 | 223 | 562 |
| 91 | 284 | 667 |
| 92 | 2253 | — |
| 93 | 1356 | 191 |
| 94 | 106 | 59 |
| 95 | 215 | 268 |
| 96 | 87 | 83 |
| 97 | 13 | 8 |
| 98 | 55 | 91 |
| 99 | 2993 | 5720 |
| 100 | 161 | 56 |
| 101 | 39 | 18 |
| 102 | 63 | 141 |
| 103 | 111 | 157 |
| 104 | 1720 | 2290 |
| 105 | 6638 | 6910 |
| 106 | >10000 | 25700 |
| 107 | >10000 | — |
| 108 | 1043 | 825 |
| 109 | 7944 | — |
| 110 | >10000 | — |
| 111 | >10000 | — |
| 112 | >10000 | 14900 |
| 113 | 8313 | 10400 |
| 114 | 3560 | 5390 |
| 115 | >10000 | — |
| 116 | >10000 | 16400 |
| 117 | >10000 | — |
| 118 | >10000 | 18000 |
| 119 | 191 | 179 |
| 120 | >10000 | 6600 |
| 121 | 26 | 63 |
| 122 | 88 | 258 |
| 123 | 303 | 25 |
| 124 | 876 | 129 |
| 125 | 33 | 68 |
| 126 | 96 | 117 |
| 127 | 34 | 12 |
| 128 | 19 | 8 |
| 129 | 355 | 415 |
| 130 | 130 | 151 |
| 131 | 32 | 57 |
| 132 | 57 | 123 |
| 133 | 17 | 40 |
| 134 | 939 | 4810 |
| 135 | >10000 | — |
| 136 | 344 | 1110 |
| 137 | 202 | 696 |
| 138 | 342 | 557 |
| 139 | 3270 | — |
| 140 | 57 | 27 |
| 141 | 1094 | 3440 |
| 142 | 491 | 890 |
| 143 | 126 | 242 |
| 144 | 98 | 102 |
| 145 | 195 | 152 |
| 146 | 27 | 21 |
| 147 | 1106 | 2040 |
| 148 | 156 | 69 |
| 149 | 70 | 29 |
| 150 | 13 | 13 |
| 151 | 226 | 10 |
| 152 | 328 | 16 |
| 153 | 195 | — |
| 154 | 34 | 44 |
| 155 | 72 | 89 |
| 156 | 19 | 11 |
| 157 | 290 | 494 |
| 158 | 797 | 1570 |
| 159 | 164 | 53 |
| 160 | 129 | 121 |
| 161 | 192 | 159 |
| 162 | 261 | — |
| 163 | 888 | 952 |
| 164 | 410 | 449 |
| 165 | >10000 | — |
| 166 | 34 | 179 |
| 167 | 54 | 115 |
| 168 | 420 | 290 |
| 169 | 141 | 24 |
| 170 | 43 | 8 |
| 171 | 1366 | 3590 |
| 172 | 617 | 1590 |
| 173 | 6 | 8 |
| 174 | 32 | 17 |
| 175 | 124 | 46 |
| 176 | 381 | 270 |
| 177 | 656 | 334 |
| 178 | 23 | 16 |
| 179 | 156 | 104 |
| 180 | 467 | 395 |
| 181 | 165 | 68 |
| 182 | 266 | 111 |
| 183 | 250 | 136 |
| 184 | 69 | 47 |
| 185 | 62 | 35 |
| 186 | 27 | — |
| 187 | 41 | — |
| 188 | 106 | 37 |
| 189 | 37 | 36 |
| 190 | 979 | — |
| 191 | 179 | 36 |
| 192 | 839 | 793 |
| 193 | 3933 | 11100 |
| 194 | 373 | 286 |
| 195 | 57 | 80 |
| 196 | 105 | 169 |
| 197 | 21 | 30 |
| 198 | 32 | 67 |
| 199 | 22 | 106 |
| 200 | 47 | 56 |
| 201 | 908 | 1610 |
| 202 | 103 | 45 |
| 203 | 252 | 224 |
| 204 | 43 | 23 |
| 205 | 266 | 107 |
| 206 | 838 | 1140 |
| 207 | 1037 | 4670 |
| 208 | 146 | 30 |
| 209 | 5220 | 533 |
| 210 | 245 | 294 |
| 211 | 953 | 1600 |
| 212 | 71 | 748 |
| 213 | 456 | 227 |
| 214 | 975 | 1850 |
| 215 | 429 | 785 |
| 216 | 130 | 220 |
| 217 | 801 | 2000 |
| 218 | 1238 | 1250 |
| 219 | 98 | 33 |
| 220 | 544 | 63 |
| 221 | 713 | 1700 |
| 222 | 40 | 19 |
| 223 | 2271 | 1940 |
| 224 | 619 | 237 |
| 225 | 372 | 312 |
| 226 | 2346 | 953 |
| 227 | 201 | 76 |
| 228 | 174 | 597 |
| 229 | 587 | 421 |
| 230 | 6477 | — |
| 231 | 73 | 84 |
| 232 | 712 | 1050 |
| 233 | 899 | 6300 |
| 234 | 116 | 215 |
| 235 | 435 | 310 |
| 236 | 1306 | 2320 |
| 237 | 14 | 9 |
| 238 | 70 | 93 |
| 239 | 121 | 67 |
| 240 | 276 | 521 |
| 241 | 162 | 233 |
| 242 | 106 | 505 |
| 243 | 302 | 539 |
| 244 | 64 | 91 |
| 245 | 68 | 113 |
| 246 | 221 | 347 |
| 247 | 215 | 53 |
| 248 | 71 | 67 |
| 249 | 60 | 30 |
| 250 | 303 | 386 |
| 251 | 32 | 29 |
| 252 | 139 | 65 |
| 253 | 15 | 18 |
| 254 | 203 | 207 |
| 255 | 1572 | 1660 |
| 256 | 26 | 13 |
| 257 | 196 | 104 |
| 258 | 195 | 53 |
| 259 | 473 | 601 |
| 260 | 332 | 201 |
| 261 | 281 | 142 |
| 262 | 92 | 20 |
| 263 | 5348 | 909 |
| 264 | >10000 | 98 |
| 265 | 121 | 93 |
| 266 | 404 | 93 |
| 267 | 520 | 113 |
| 268 | 53 | 34 |
| 269 | 163 | 92 |
| 270 | 19 | 8 |
| 271 | 86 | 25 |
| 272 | 405 | 479 |
| 273 | 257 | 513 |
| 274 | 486 | 135 |
| 275 | 371 | 176 |
| 276 | 2892 | 185 |
| 277 | 389 | 67 |
| 278 | 575 | 282 |
| 279 | 849 | 208 |
| 280 | 233 | 91 |
| 281 | 102 | 43 |
| 282 | 54 | 26 |
| 283 | 13 | 23 |
| 284 | 45 | 35 |
| 285 | 63 | 13 |
| 286 | 270 | 122 |
| 287 | 116 | 21 |
| 288 | 180 | 33 |
| 289 | 91 | 12 |
| 290 | 67 | 279 |
| 291 | 172 | 257 |
| 292 | 149 | 199 |
| 293 | 404 | 810 |
| 294 | 66 | 138 |
| 295 | 281 | 327 |
| 296 | 144 | 175 |
| 297 | 50 | 69 |
| 298 | 163 | 166 |
| 299 | 758 | 1372 |
| 300 | 227 | 283 |
| 301 | 284 | 273 |
| 302 | 97 | 95 |
| 303 | 715 | 123 |
| 304 | 167 | 202 |
| 305 | 35 | 68 |
| 306 | 110 | 150 |
| 307 | 421 | 41 |
| 308 | 99 | 97 |
| 309 | 113 | 145 |
| 310 | 328 | 501 |
| 311 | 94 | 58 |
| 312 | 124 | 145 |
| 313 | 196 | 137 |
| 314 | 210 | 73 |
| 315 | 268 | 224 |
| 316 | 106 | 93 |
| 317 | 456 | 745 |
| 318 | 175 | 37 |
| 319 | 220 | 62 |
| 320 | 127 | 88 |
| 321 | 307 | 568 |
| 322 | 435 | 314 |
| 323 | 142 | 297 |
| 324 | 481 | 269 |
| 325 | 1214 | 2018 |
| 326 | 506 | 105 |
| 327 | 158 | 128 |
| 328 | 199 | 56 |
| 329 | 395 | 71 |
| 330 | 303 | 2965 |
| 331 | 169 | 68 |
| 332 | 151 | 477 |
| 333 | 124 | 1838 |
| 334 | 156 | 65 |
| 335 | 167 | 75 |
| 336 | 173 | 587 |
| 337 | 623 | 1281 |
| 338 | 303 | 488 |
| 339 | 222 | 735 |
| 340 | 161 | 477 |
| 341 | 1240 | 5174 |
| 342 | 295 | 1063 |
| 343 | 168 | 30 |
| 344 | 68 | 140 |
| 345 | 282 | 115 |
| 346 | 397 | 55 |
| 347 | 742 | 2248 |
| 348 | 1194 | 5875 |
| 349 | 67 | 55 |
| 350 | 21 | 94 |
| 351 | 66 | 386 |
| 352 | 1364 | 17026 |
| 353 | 135 | 655 |
| 354 | 146 | 492 |
| 355 | 126 | 836 |
| 356 | 37 | 487 |
| 357 | 31 | 182 |
| 358 | 445 | 1917 |
| 359 | 755 | 8433 |
| 360 | 1017 | 6575 |
| 361 | 670 | 4666 |
| 362 | 1016 | 13071 |
| 363 | 1373 | 14075 |
| 364 | 54 | 141 |
| 365 | 44 | 1289 |
| 366 | 33 | 604 |
| 367 | 97 | 2150 |
| 368 | 460 | 713 |
| 369 | 317 | 1301 |
| 370 | 82 | 243 |
| 371 | 68 | 815 |
| 372 | 32 | 229 |
| 373 | 340 | 5709 |
| 374 | 130 | 458 |
| 375 | 75 | 480 |
| 376 | 193 | 646 |
| 377 | 62 | 449 |
| 378 | 1282 | 3350 |
| 379 | 118 | 1108 |
| 380 | 318 | 11000 |
| 381 | 22 | 385 |
| 382 | 45 | 400 |
| 383 | — | 385 |
| 384 | 90 | 456 |
| 385 | 41 | 79 |
| 386 | 156 | 2470 |
| 387 | 1061 | 315 |
| 388 | 251 | 1990 |
| 389 | 475 | 3736 |
| 390 | 161 | 193 |
| 391 | 125 | 3450 |
| 392 | 123 | 85 |
| 393 | 414 | 2830 |
| 394 | 469 | 5810 |
| 395 | 278 | 2420 |
| 396 | 920 | 7860 |
| 397 | 188 | 457 |
| 398 | 36 | 90 |
| 399 | 97 | 801 |
| 400 | 104-165 | 684 |
| 401 | 107 | 688 |
| 402 | 522 | 1660 |
| 403 | 84 | 549 |
| 404 | 72 | 778 |
| 405 | 49 | 70 |
| 406 | 235 | 465 |
| 407 | 414 | 1150 |
| 408 | 227 | 331 |
| 409 | 63 | 6540 |
| 410 | 257 | 3370 |
| 411 | 377 | 1590 |
| 412 | 427 | 3790 |
| 413 | 226 | 2070 |
| 414 | 103 | 688 |
| 415 | 133 | 1141 |
| 416 | 64 | 770 |
| 417 | 99 | 1475 |
| 418 | 40 | 238 |
| 419 | 179 | 1576 |
| 420 | 59 | 324 |
| 421 | 454 | 2561 |
| 422 | 88 | 149 |
| 423 | 65 | 124 |
| 424 | 308 | 157 |
| 425 | 113 | 3124 |
| 426 | 10 | 69 |
| 427 | 37 | 199 |
| 428 | 5 | 28 |
| 429 | 30 | 97 |
| 430 | 661 | 16068 |
| 431 | — | 535 |
| 432 | 16-43 | 3866 |
| 433 | 78 | 2461 |
| 434 | 158 | 6852 |
| 435 | 41 | 25 |
| 436 | 38-88 | 4502 |
| 437 | 107 | 518 |
| 438 | 110 | 1767 |
| 439 | 194 | 3646 |
| 440 | 59 | 105 |
| 441 | 65 | 289 |
| 442 | 110 | 705 |
| 443 | 9 | 74 |
| 444 | 51 | 199 |
| 445 | — | — |
| 446 | — | 487 |
| 447 | 9 | 82 |
| 448 | — | 175 |
| 449 | — | 280 |
| 450 | — | 10513 |
| 451 | 19 | 59 |
| 452 | 3556 | 19925 |
| 453 | 82 | 1350 |
| 454 | 169 | 2501 |
| 455 | — | 6465 |
| 456 | 872 | 2951 |
| 457 | 121 | 408 |
| 458 | 75 | 117 |
| 459 | 58 | 273 |
| 460 | 9 | 43 |
| 461 | — | 244 |

“—” denotes that the values were not measured or that no value was determined

The data in Table 2 indicate that most of the compounds synthesised are active in the micromolar or nanomolar concentration range in cell-based and cell-free systems.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A compound, being of Formula (I) 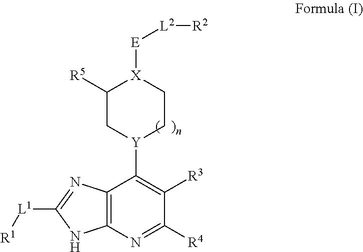
or a pharmaceutically acceptable salt thereof, wherein:
R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
wherein each R^{A} is independently selected from halo, —OH, —NHR′, —CN, oxo, —SO₂CH₃, —(C₁-C₃)alkyl, —O(C₁-C₃)alkyl, —C(O)R″, —C(O)NHR′, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, —NH₂, and —CN,
and/or wherein two occurrences of R^{A} may be taken together with the atom(s) to which they are attached to form a (C₃-C₆)cycloalkyl group, or a 3- to 6-membered heterocycloalkyl group,
wherein each R′ is independently selected from —H, methyl, and phenyl, and
wherein each R″ is independently selected from —OH, methyl, phenyl optionally substituted by —OCF₃, and tetrahydrofuranyl;
L¹ is selected from a direct bond, —O—, —CH₂—, and —CH═;
R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
wherein each R^{B} is selected from halo, —OH, oxo, —NH₂, —NHMe, —NO₂, —CN, —SF₅, —SiMe₃, —B(OH)₂, —(C₁-C₃)alkyl, —O(C₁-C₃)alkyl, —(C₃-C₆)cycloalkyl, —SO₂CH₃, —NHC(O)Me, phenyl, benzyl, 1-λ⁶-2-thiazolidine-1,1-dionyl, and pyridyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
and further wherein, when R² is —(C₃-C₆)cycloalkyl, R² is optionally substituted at one ring atom by a spirocyclic oxindolyl group;
L² is selected from a direct bond, —CH₂—, —CH₂CH₂—, and —CH₂O—, wherein L², when present, is optionally substituted with —NH₂ or methyl;
R³ is selected from —H, halo, —OH, and methyl optionally substituted with —OH;
R⁴ is —H, or —NH₂;
R⁵ is —H, or methyl;
X is CH or N;
Y is selected from CH, CF, and N (with the proviso that when Y is N, X is also N);
E is selected from —C(O)—, —S(O)₂—, and —O— (with the proviso that when E is —O—, X and Y are both CH); and
n is 0, 1, or 2.

2. The compound of claim 1, wherein the compound is a compound of Formula (I-A) 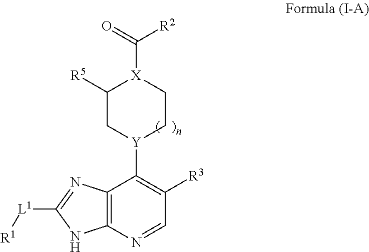
or a pharmaceutically acceptable salt thereof, wherein R¹, R², R³, R⁵, L¹, X, Y, and n are as defined in claim 1.

3. The compound of claim 1 or claim 2, wherein the compound is a compound of Formula (I-B) 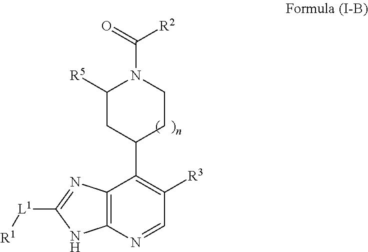
or a pharmaceutically acceptable salt thereof, wherein R¹, R², R³, R⁵, L¹, and n are as defined in any of the preceding claims.

4. The compound of any one of claims 1 to 3, wherein:
R¹ is selected from —H, —(C₁-C₆)alkyl, —(C₃-C₇)cycloalkyl, —(C₄-C₈)cycloalkenyl, —(C₆-C₁₀)aryl, 4- to 10-membered heterocycloalkyl, 5- to 8-membered heterocycloalkenyl, and 5- to 10-membered heteroaryl, wherein R¹ is optionally substituted with one or more occurrences of R^{A},
wherein each R^{A} is independently selected from halo, —OH, —NHR′, —CN, oxo, —SO₂CH₃, —(C₁-C₃)alkyl, —O(C₁-C₃)alkyl, —C(O)R″, —C(O)NHR′, —NHC(O)R″, —(C₃-C₆)cycloalkyl, 3- to 6-membered heterocycloalkyl, phenyl, and pyridyl, wherein each occurrence of —(C₁-C₃)alkyl, —O(C₁-C₃)alkyl, and phenyl, is optionally substituted by one or more groups independently selected from halo, —OH, —NH₂, and —CN,
and/or wherein two occurrences of R^{A} may be taken together with the atom(s) to which they are attached to form a (C₃-C₆)cycloalkyl group, or a 3- to 6-membered heterocycloalkyl group,
wherein each R′ is independently selected from —H, methyl, and phenyl, and
wherein each R″ is independently selected from methyl, phenyl optionally substituted by —OCF₃, and tetrahydrofuranyl;
R² is selected from —(C₃-C₆)cycloalkyl, 5- to 10-membered heterocycloalkyl, —(C₆-C₁₀)aryl, and 5- to 10-membered heteroaryl, wherein R² is optionally substituted with one or more occurrences of R^{B},
wherein each R^{B} is selected from halo, —OH, —NH₂, —NO₂, —CN, —SF₅, —B(OH)₂, —(C₁-C₃)alkyl, —O(C₁-C₃)alkyl, —(C₃-C₆)cycloalkyl, —SO₂CH₃, phenyl, benzyl, 1-λ⁶-2-thiazolidine-1,1-dionyl, and pyridyl, wherein each occurrence of —(C₁-C₃)alkyl, —O(C₁-C₃)alkyl, —(C₃-C₆)cycloalkyl, phenyl, and benzyl, is optionally substituted by one or more groups independently selected from halo, —OH, —NH₂, and —CF₃,
and further wherein, when R² is —(C₃-C₆)cycloalkyl, R² is optionally substituted at one ring atom by a spirocyclic oxindolyl group; and
X and Y are each independently selected from CH, and N (with the proviso that when Y is N, X is also N).

5. The compound of any one of claims 1 to 4, wherein the compound is a compound of Formula (I-C) or Formula (I-D) 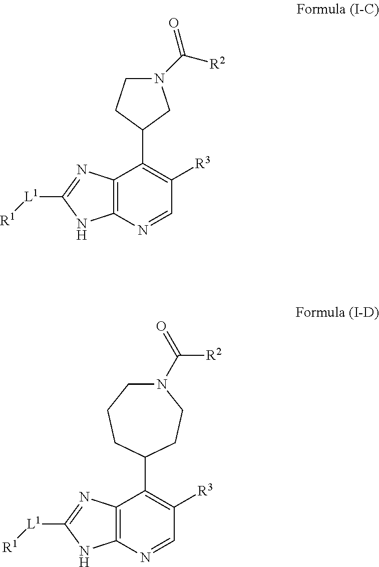
or a pharmaceutically acceptable salt thereof, wherein R¹, R², R³, and L¹ are as defined in any of the preceding claims.

6. The compound of any one of claims 1 to 5, wherein R² is selected from —(C₃-C₆)cycloalkyl, 5- to 6-membered heterocycloalkyl, 9- to 10-membered heterocycloalkyl, —(C₆-C₁₀)aryl, 5- to 6-membered heteroaryl, and 9- to 10-membered heteroaryl, wherein R² is optionally substituted with one or more occurrences of R^{B} as defined in any of the preceding claims.

7. The compound of any one of claims 1 to 6, wherein R² is selected from: 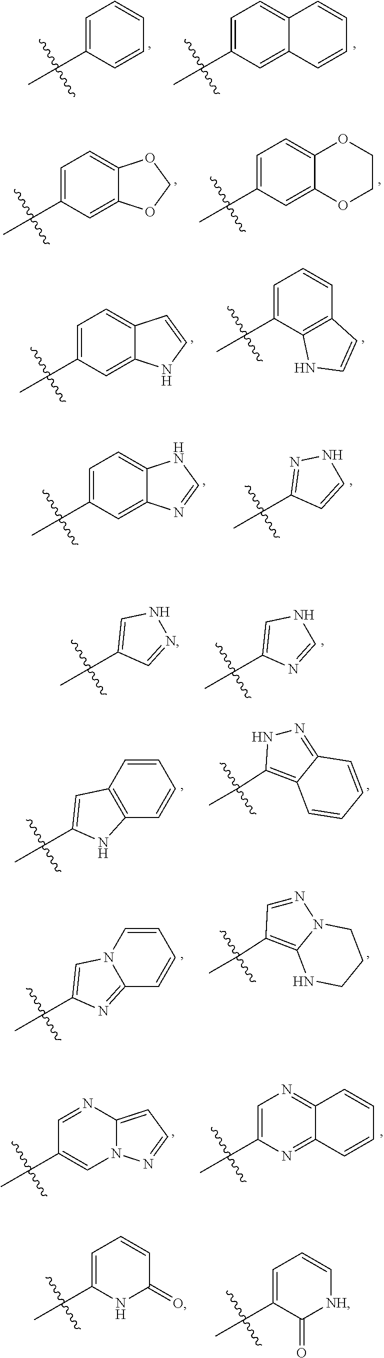 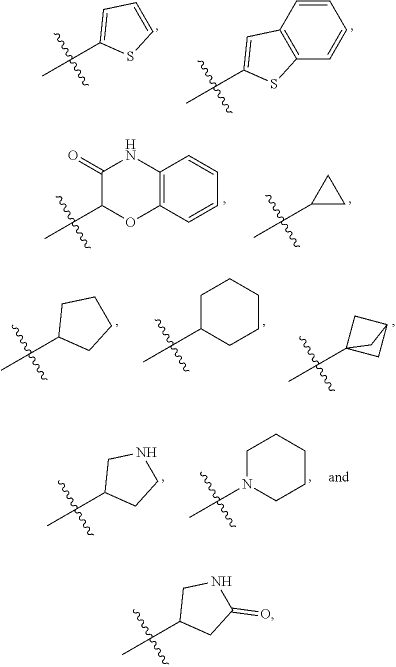
wherein R² is optionally substituted by one or more occurrences of R^{B} as defined in any of the preceding claims.

8. The compound of any one of claims 1 to 7, wherein R² is selected from: 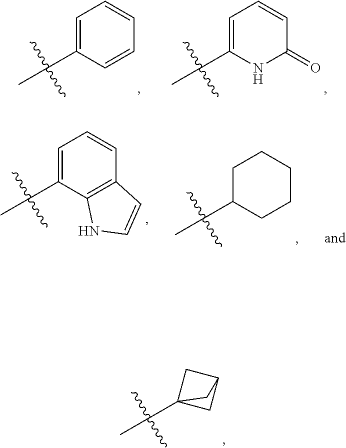
wherein R² is optionally substituted by one or more occurrences of R^{B}, wherein each R^{B} is independently selected from the group consisting of halo, —NH₂, —O(C₁-C₃)alkyl, —(C₁-C₃)alkyl, —(C₃-C₆)cycloalkyl, —SF₅, and —NO₂, and wherein each occurrence of —O(C₁-C₃)alkyl, —(C₁-C₃)alkyl, and —(C₃-C₆)cycloalkyl, is optionally substituted by one or more groups independently selected from halo and —CF₃.

9. The compound of any one of claims 1 to 8, wherein at least one occurrence of R^{B} is —SF₅.

10. The compound of any one of claims 1 to 9, wherein R² is:

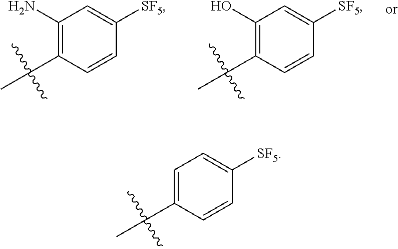

11. The compound of any one of claims 1 to 10, wherein R¹ is selected from —H, —(C₁-C₃)alkyl, —(C₃-C₆)cycloalkyl, —(C₅-C₇)cycloalkenyl, —(C₆-C₁₀)aryl, 4- to 9-membered heterocycloalkyl, 6-membered heterocycloalkenyl, 5- to 6-membered heteroaryl, and 9- to 10-membered heteroaryl, wherein R¹ is optionally substituted with one or more occurrences of R^{A} as defined in any of the preceding claims.

12. The compound of any one of claims 1 to 11, wherein R′ is selected from: 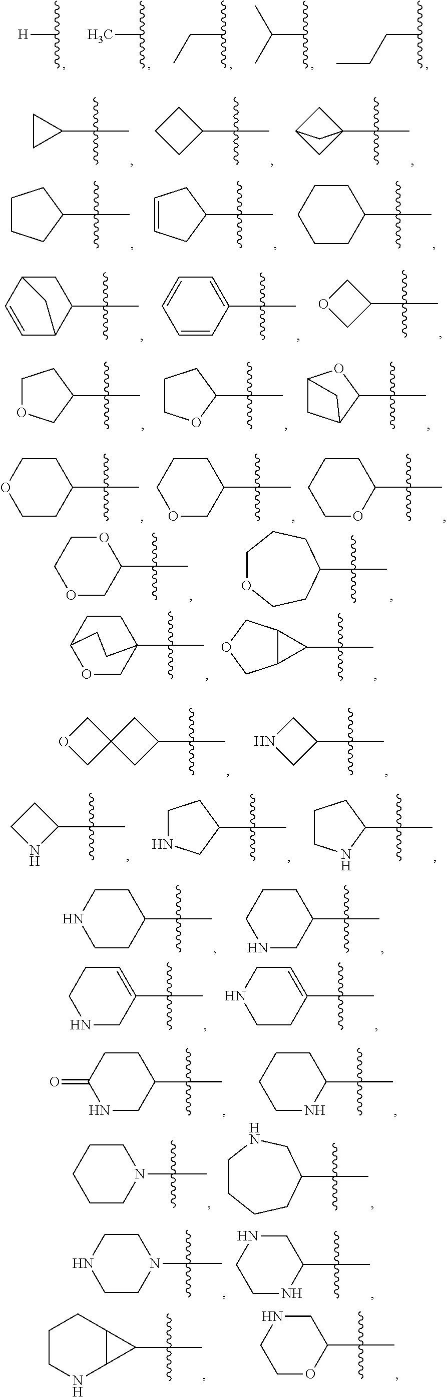 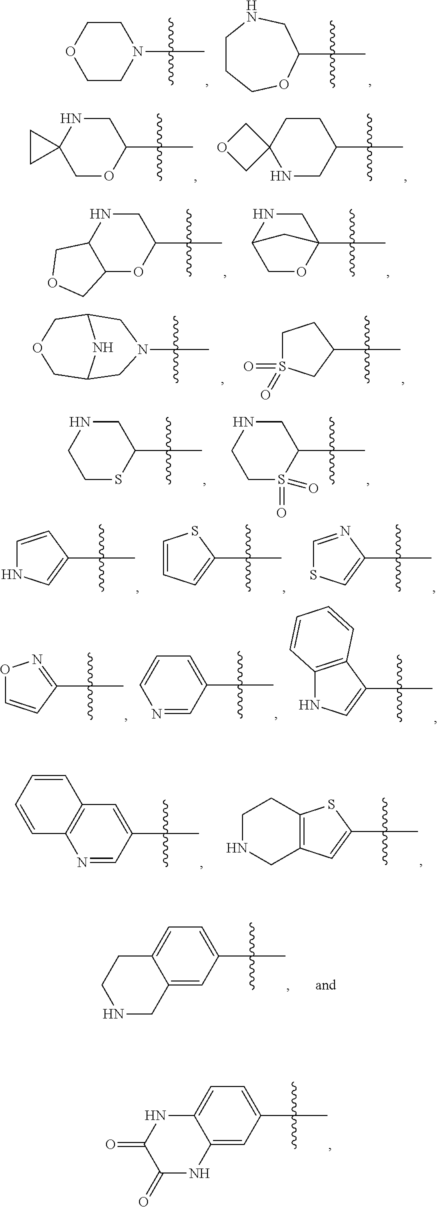
wherein R¹ is optionally substituted by one or more occurrences of R^{A} as defined in any of the preceding claims.

13. The compound of any one of claims 1 to 12, wherein R′ is selected from: 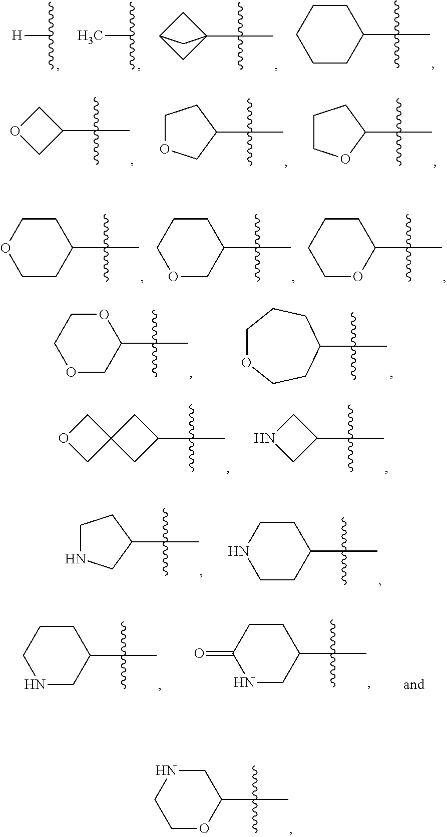
wherein R¹ is optionally substituted by one or more occurrences of R^{A}, wherein each R^{A} is independently selected from the group consisting of -halo, —OH, —NH₂, oxo, —(C₁-C₃)alkyl, —C(O)R″, and —O(C₁-C₃)alkyl, wherein each occurrence of (C₁-C₃)alkyl may be optionally substituted by one or more halo groups, and wherein each R″ is independently selected from methyl, phenyl optionally substituted by —OCF₃, and tetrahydrofuranyl.

14. The compound of any one of claims 1 to 13, wherein R¹ is selected from —(C₄-C₈)cycloalkenyl, —(C₆-C₁₀)aryl, 7- to 10-membered multicyclic (e.g., bicyclic) heterocycloalkyl, 5- to 8-membered heterocycloalkenyl, and 7- to 10-membered multicyclic (e.g., bicyclic) heteroaryl, wherein R¹ is optionally substituted with one or more occurrences of R^{A} as defined in any of the preceding claims, or R¹ is —(C₁-C₆)alkyl substituted by one or more substituents selected from —OH, —NH₂, and —O(C₁-C₃)alkyl.

15. The compound of any one of claims 1 to 14, wherein L¹ is a direct bond or —CH₂—.

16. A compound, being of Formula (II) 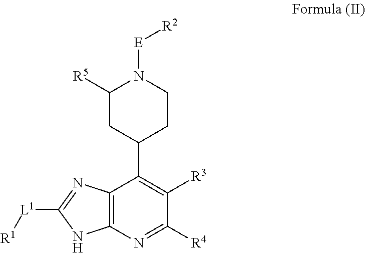
or a pharmaceutically acceptable salt thereof, wherein R¹, L¹, R³, R⁴, and R⁵ are as defined in any of the preceding claims, and wherein:
R² is selected from —(C₃-C₆)cycloalkyl (e.g., cyclopropyl), —(C₆-C₁₀)aryl, 5- to 10-membered heterocycloalkyl, and 5- to 10-membered heteroaryl, wherein R² is optionally substituted by one or more occurrences of R^{B}, wherein each R^{B} is independently selected from the group consisting of halo, —OH, oxo, —NO₂, —NH₂, —NHMe, —CN, —SF₅, —SO₂CH₃, —NHC(O)Me, —SiMe₃, —(C₁-C₃)alkyl optionally substituted by one or more halo, —O(C₁-C₃)alkyl optionally substituted by one or more halo, —(C₃-C₆)cycloalkyl optionally substituted by —CF₃, phenyl optionally substituted by one or more halo, —OH, or —CF₃, benzyl optionally substituted by halo, and 1-λ⁶-2-thiazolidine-1,1-dionyl,
and further wherein, when R² is cyclopropyl, R² is optionally substituted at one ring atom by a spirocyclic oxindolyl group; and
E is —C(O)— or —S(O)₂—.

17. A compound, being of Formula (III) 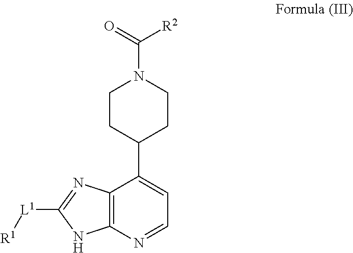
or a pharmaceutically acceptable salt thereof, wherein:
R¹ is —H, —(C₁-C₃)alkyl, —(C₄-C₆)cycloalkyl, —(C₅-C₇)cycloalkenyl, —(C₆-C₁₀)aryl, or 4- to 9-membered heterocycloalkyl, 6-membered heterocycloalkenyl, 5- to 6-membered heteroaryl, and 9- to 10-membered heteroaryl, wherein R¹ is optionally substituted by one or more R^{A}, wherein each R^{A} is independently selected from halo, —OH, oxo, —NH₂, —NHMe, —C(O)OH, —C(O)Me, —SO₂CH₃, —NHC(O)R″, —(C₁-C₃)alkyl, —O(C₁-C₃)alkyl, cyclopropyl, oxetanyl, sulfolanyl, phenyl, benzyl, and pyridyl, wherein each occurrence of —(C₁-C₃)alkyl, —O(C₁-C₃)alkyl, and phenyl is optionally substituted by one or more groups independently selected from halo, —OH, and —NH₂,
wherein each R″ is independently selected from methyl, and tetrahydrofuranyl;
L¹ is a direct bond or —CH₂—; and
R² is selected from —(C₃-C₆)cycloalkyl, —(C₆-C₁₀)aryl, 5- to 6-membered heterocycloalkyl, and 5- to 10-membered heteroaryl, wherein R² is optionally substituted by one or more occurrences of R^{B}, wherein each R^{B} is independently selected from the group consisting of halo, —NO₂, —NH₂, —NHMe, —NHAc, —O(C₁-C₃)alkyl, —SF₅, —SiMe₃, —(C₁-C₃)alkyl, 1-λ⁶-2-thiazolidine-1,1-dionyl, phenyl, benzyl, and —(C₃-C₆)cycloalkyl optionally substituted by —CF₃, wherein each occurrence of —(C₁-C₃)alkyl, —O(C₁-C₃)alkyl, phenyl, and benzyl, is optionally substituted by one or more groups independently selected from halo, —NH₂, and —CF₃, and further wherein, when R² is cyclopropyl, R² is optionally substituted at one ring atom by a spirocyclic oxindolyl group.

18. A compound, being of Formula (IV) 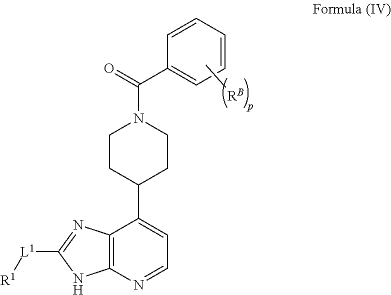
or a pharmaceutically acceptable salt thereof, wherein R¹, and L¹ are as defined in any of the preceding claims, and wherein:
p is 1, 2, 3, 4, or 5; and
each R^{B} is independently selected from the group consisting of halo, —NO₂, —NH₂, —NHMe, —NHAc, —OCH₃, —OCF₃, —SF₅, SiMe₃, (C₁-C₃)alkyl, —O(C₁-C₃)alkyl, 1-λ⁶-2-thiazolidine-1,1-dionyl, phenyl, and —(C₃-C₆)cycloalkyl optionally substituted by —CF₃, wherein each occurrence of —(C₁-C₃)alkyl, —O(C₁-C₃)alkyl, and phenyl is optionally substituted by one or more groups independently selected from halo, —NH₂, and —CF₃.

19. A compound, being of Formula (V) 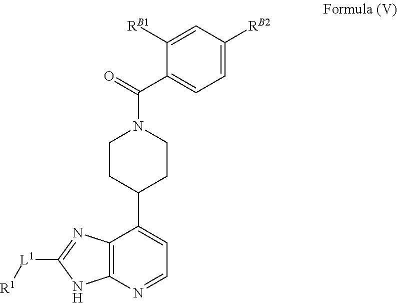
or a pharmaceutically acceptable salt thereof, wherein R¹, and L¹ are as defined in any of the preceding claims, and wherein:
R^{B1} is either —H, or is selected from the group consisting of halo (e.g. —F, or —Cl), —OH, —NH₂, —NHMe, —NHAc, and —NO₂; and
R^{B2} is selected from the group consisting of halo (e.g. —Br), —SF₅, —SO₂CH₃, —SiMe₃, —(C₁-C₃)alkyl optionally substituted by one or more halo groups, —O(C₁-C₃)alkyl optionally substituted by one or more halo groups, 1-trifluoromethylcyclopropan-1-yl, and phenyl optionally substituted by one or more groups independently selected from halo, or —NH₂.

20. The compound of claim 19, wherein R^{B2} is —SF₅.

21. A compound, being of Formula (VIII), (IX), or (X) 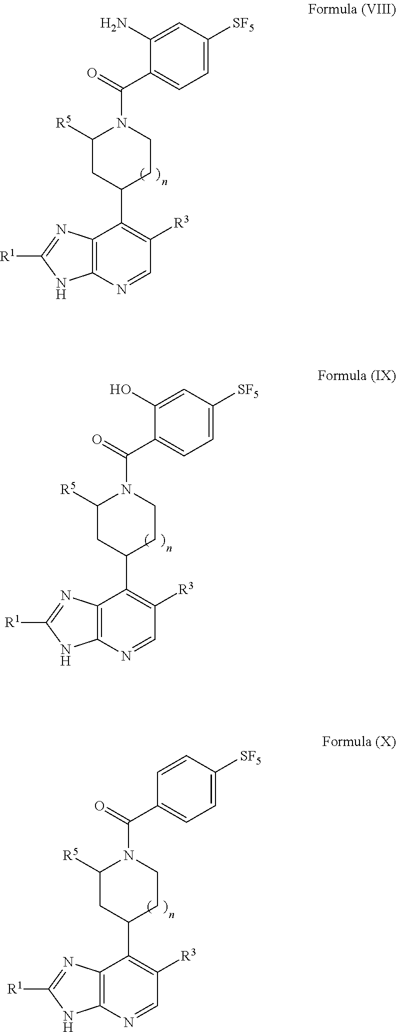
or a pharmaceutically acceptable salt thereof, wherein R¹, R³, R⁵, and n are as defined in any of the preceding claims.

22. A compound selected from the group consisting of:
[2-amino-4-(trifluoromethoxy)phenyl]-[4-[2-(tetrahydrofuran-3-ylmethyl)-3H-imidazo[4,5-b]pyridin-7-yl]-1-piperidyl]methanone,
[2-amino-4-(trifluoromethoxy)phenyl]-[4-(2-tetrahydrofuran-3-yl-3H-imidazo[4,5-b]pyridin-7-yl)-1-piperidyl]methanone,
[2-amino-4-(trifluoromethoxy)phenyl]-[4-(2-tetrahydropyran-4-yl-3H-imidazo[4,5-b]pyridin-7-yl)-1-piperidyl]methanone,
[4-[2-(4-piperidyl)-3H-imidazo[4,5-b]pyridin-7-yl]-1-piperidyl]-[4-(trifluoromethoxy)phenyl]methanone,
trans-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-hydroxycyclohexyl)-3H-imidazo[4,5-b]pyridin-7-yl]-1-piperidyl]-[4-(trifluoromethoxy)phenyl]methanone,
cis-[4-[2-(4-aminocyclohexyl)-3H-imidazo[4,5-b]pyridin-7-yl]-1-piperidyl]-[4-(trifluoromethoxy)phenyl]methanone,
1-[3-[7-[1-[4-(trifluoromethoxy)benzoyl]-4-piperidyl]-3H-imidazo[4,5-b]pyridin-2-yl]azetidin-1-yl]ethanone,
[4-(2-tetrahydropyran-4-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(1-methyl-4-piperidyl)-3H-imidazo[4,5-b]pyridin-7-yl]-1-piperidyl]methanone,
[4-[2-(1-methyl-4-piperidyl)-3H-imidazo[4,5-b]pyridin-7-yl]-1-piperidyl]-[4-(trifluoromethoxy)phenyl]methanone,
[4-(2-methyl-3H-imidazo[4,5-b]pyridin-7-yl)-1-piperidyl]-[4-(trifluoromethoxy)phenyl]methanone,
[4-[2-(3-methoxy-1-bicyclo[1.1.1]pentanyl)-3H-imidazo[4,5-b]pyridin-7-yl]-1-piperidyl]-[4-(trifluoromethoxy)phenyl]methanone,
5-[7-[1-[4-(trifluoromethoxy)benzoyl]-4-piperidyl]-3H-imidazo[4,5-b]pyridin-2-yl]piperidin-2-one,
[4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone,
[4-(3H-imidazo[4,5-b]pyridin-7-yl)-1-piperidyl]-[4-(trifluoromethoxy)phenyl]methanone,
[4-[2-(oxetan-3-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(2,2-dimethyltetrahydropyran-4-yl)-3H-imidazo[4,5-b]pyridin-7-yl]-1-piperidyl]methanone,
[4-[2-(azetidin-3-yl)-3H-imidazo[4,5-b]pyridin-7-yl]-1-piperidyl]-[4-(trifluoromethoxy)phenyl]methanone,
[4-(2-morpholin-2-yl-3H-imidazo[4,5-b]pyridin-7-yl]-1-piperidyl]-[4-(trifluoromethoxy)phenyl]methanone,
[4-[2-(1-methylpyrrolidin-3-yl)-3H-imidazo[4,5-b]pyridin-7-yl]-1-piperidyl]-[4-(trifluoromethoxy)phenyl]methanone,
[4-(2-tetrahydrofuran-3-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-[2-(6,6-dimethyltetrahydropyran-3-yl)-3H-imidazo[4,5-b]pyridin-7-yl]-1-piperidyl]methanone,
[2-amino-4-(trifluoromethoxy)phenyl]-[4-(2-morpholin-2-yl-3H-imidazo[4,5-b]pyridin-7-yl)-1-piperidyl]methanone,
[4-[2-(2,2-dimethyltetrahydropyran-4-yl)-3H-imidazo[4,5-b]pyridin-7-yl]-1-piperidyl]-[4-(trifluoromethoxy)phenyl]methanone,
[4-[2-(6,6-dimethyltetrahydropyran-3-yl)-3H-imidazo[4,5-b]pyridin-7-yl]-1-piperidyl]-[4-(trifluoromethoxy)phenyl]methanone,
[4-[2-(1,4-dioxan-2-yl)-3H-imidazo[4,5-b]pyridin-7-yl]-1-piperidyl]-[4-(trifluoromethoxy)phenyl]methanone,
[4-(2-tetrahydrofuran-2-yl-3H-imidazo[4,5-b]pyridin-7-yl)-1-piperidyl]-[4-(trifluoromethoxy)phenyl]methanone,
[4-[2-(4-methylmorpholin-2-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(5,5-difluoro-3-piperidyl)-3H-imidazo[4,5-b]pyridin-7-yl]-1-piperidyl]methanone,
[2-amino-4-(trifluoromethoxy)phenyl]-[4-[2-[1-(2,2-difluoroethyl)-4-piperidyl]-3H-imidazo[4,5-b]pyridin-7-yl]-1-piperidyl]methanone,
[4-[2-[1-(2,2-difluoroethyl)azetidin-3-yl]-3H-imidazo[4,5-b]pyridin-7-yl]-1-piperidyl]-[4-(trifluoromethoxy)phenyl]methanone,
[4-[2-(oxetan-3-ylmethyl)-3H-imidazo[4,5-b]pyridin-7-yl]-1-piperidyl]-[4-(trifluoromethoxy)phenyl]methanone,
(4-chloro-1H-indol-7-yl)-[4-(2-tetrahydropyran-4-yl-3H-imidazo[4,5-b]pyridin-7-yl)-1-piperidyl]methanone,
[2-nitro-4-(pentafluoro-λ⁶-sulfanyl)phenyl]-[4-(2-tetrahydropyran-4-yl-3H-imidazo[4,5-b]pyridin-7-yl)-1-piperidyl]methanone,
[4-[2-(tetrahydrofuran-3-ylmethyl)-3H-imidazo[4,5-b]pyridin-7-yl]-1-piperidyl]-[4-(trifluoromethoxy)phenyl]methanone,
[4-[2-(oxepan-4-yl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(oxepan-4-yl)-3H-imidazo[4,5-b]pyridin-7-yl]-1-piperidyl]methanone,
[4-[2-(2-oxaspiro[3.3]heptan-6-ylmethyl)-3H-imidazo[4,5-b]pyridin-7-yl]-1-piperidyl]-[4-(trifluoromethoxy)phenyl]methanone,
[4-(1,1,2,2,2-pentafluoroethyl)phenyl]-[4-(2-tetrahydropyran-4-yl-3H-imidazo[4,5-b]pyridin-7-yl)-1-piperidyl]methanone,
[4-(pentafluoro-λ⁶-sulfanyl)phenyl]-[4-(2-tetrahydropyran-4-yl-3H-imidazo[4,5-b]pyridin-7-yl)-1-piperidyl]methanone,
(cis)-[4-(2-tetrahydropyran-4-yl-3H-imidazo[4,5-b]pyridin-7-yl)-1-piperidyl]-[4-(2,2,2-trifluoroethyl)cyclohexyl]methanone,
(trans)-[4-(2-tetrahydropyran-4-yl-3H-imidazo[4,5-b]pyridin-7-yl)-1-piperidyl]-[4-(2,2,2-trifluoroethyl)cyclohexyl]methanone,
[2-amino-4-(trifluoromethoxy)phenyl]-[4-[2-(2-oxaspiro[3.3]heptan-6-ylmethyl)-3H-imidazo[4,5-b]pyridin-7-yl]-1-piperidyl]methanone,
[2-amino-4-(trifluoromethoxy)phenyl]-[4-[2-(oxetan-3-ylmethyl)-3H-imidazo[4,5-b]pyridin-7-yl]-1-piperidyl]methanone,
(4-methoxy-1H-indol-7-yl)-[4-(2-tetrahydropyran-4-yl-3H-imidazo[4,5-b]pyridin-7-yl)-1-piperidyl]methanone,
6-[4-(2-tetrahydropyran-4-yl-3H-imidazo[4,5-b]pyridin-7-yl)piperidine-1-carbonyl]-3-(trifluoromethyl)-1H-pyridin-2-one,
[4-(2-tetrahydropyran-4-yl-3H-imidazo[4,5-b]pyridin-7-yl)-1-piperidyl]-[4-(2,2,2-trifluoroethyl)phenyl]methanone,
[4-(trifluoromethoxy)phenyl]-[4-[2-(trifluoromethyl)-3H-imidazo[4,5-b]pyridin-7-yl]-1-piperidyl]methanone,
[4-(2-tetrahydropyran-2-yl-3H-imidazo[4,5-b]pyridin-7-yl)-1-piperidyl]-[4-(trifluoromethoxy)phenyl]methanone,
[2-amino-4-(trifluoromethoxy)phenyl]-[4-(2-tetrahydropyran-2-yl-3H-imidazo[4,5-b]pyridin-7-yl)-1-piperidyl]methanone,
(3-methoxy-1-bicyclo[1.1.1]pentanyl)-[4-(2-tetrahydropyran-4-yl-3H-imidazo[4,5-b]pyridin-7-yl)-1-piperidyl]methanone,
[4-(2-tetrahydropyran-4-yl-3H-imidazo[4,5-b]pyridin-7-yl)-1-piperidyl]-[3-(trifluoromethyl)-1-bicyclo[1.1.1]pentanyl]methanone,
[4-(2-tetrahydropyran-4-yl-3H-imidazo[4,5-b]pyridin-7-yl)-1-piperidyl]-[4-[1-(trifluoromethyl)cyclopropyl]phenyl]methanone,
[4-[2-(5,5-difluoro-3-piperidyl)-3H-imidazo[4,5-b]pyridin-7-yl]-1-piperidyl]-[4-(trifluoromethoxy)phenyl]methanone,
[2-amino-4-(trifluoromethoxy)phenyl]-[4-[2-(tetrahydropyran-4-ylmethyl)-3H-imidazo[4,5-b]pyridin-7-yl]-1-piperidyl]methanone,
[4-(2-pyrrolidin-3-yl-3H-imidazo[4,5-b]pyridin-7-yl)-1-piperidyl]-[4-(trifluoromethoxy)phenyl]methanone,
[4-[2-[(1-methyl-4-piperidyl)methyl]-3H-imidazo[4,5-b]pyridin-7-yl]-1-piperidyl]-[4-(trifluoromethoxy)phenyl]methanone,
[2-amino-4-(trifluoromethoxy)phenyl]-[4-(2-tetrahydropyran-3-yl-3H-imidazo[4,5-b]pyridin-7-yl)-1-piperidyl]methanone,
[4-[2-(tetrahydropyran-4-ylmethyl)-3H-imidazo[4,5-b]pyridin-7-yl]-1-piperidyl]-[4-(trifluoromethoxy)phenyl]methanone,
[4-(2-tetrahydropyran-3-yl-3H-imidazo[4,5-b]pyridin-7-yl)-1-piperidyl]-[4-(trifluoromethoxy)phenyl]methanone, and
[4-[2-[1-(2,2-difluoroethyl)-4-piperidyl]-3H-imidazo[4,5-b]pyridin-7-yl]-1-piperidyl]-[4-(trifluoromethoxy)phenyl]methanone,
and the pharmaceutically acceptable salts thereof.

23. A compound selected from the group consisting of:
[2-amino-4-(trifluoromethoxy)phenyl]-[4-[2-(oxetan-3-yl)-3H-imidazo[4,5-b]pyridin-7-yl]-1-piperidyl]methanone;
[4-[2-[tetrahydrofuran-2-yl]-3H-imidazo[4,5-b]pyridin-7-yl]-1-piperidyl]-[4-(trifluoromethoxy)phenyl]methanone;
[4-[2-(3-hydroxy-1-bicyclo[1.1.1]pentanyl)-3H-imidazo[4,5-b]pyridin-7-yl]-1-piperidyl]-[4-(trifluoromethoxy)phenyl]methanone;
[4-[2-[pyrrolidin-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[tetrahydropyran-2-yl]-3H-imidazo[4,5-b]pyridin-7-yl]-1-piperidyl]methanone;
2-naphthyl-[4-(2-tetrahydropyran-4-yl-3H-imidazo[4,5-b]pyridin-7-yl)-1-piperidyl]methanone;
1H-indazol-3-yl-[4-(2-tetrahydropyran-4-yl-3H-imidazo[4,5-b]pyridin-7-yl)-1-piperidyl]methanone;
[4-(2-tetrahydropyran-4-yl-3H-imidazo[4,5-b]pyridin-7-yl)-1-piperidyl]-[7-(trifluoromethyl)-4,5,6,7-tetrahydropyrazolo[1,5-a]pyrimidin-3-yl]methanone;
(1-methyl-5-phenyl-pyrazol-3-yl)-[4-(2-tetrahydropyran-4-yl-3H-imidazo[4,5-b]pyridin-7-yl)-1-piperidyl]methanone;
(3-amino-2-naphthyl)-[4-(2-tetrahydropyran-4-yl-3H-imidazo[4,5-b]pyridin-7-yl)-1-piperidyl]methanone;
(2-amino-3,4,5,6-tetrafluoro-phenyl)-[4-(2-tetrahydropyran-4-yl-3H-imidazo[4,5-b]pyridin-7-yl)-1-piperidyl]methanone;
[1-isopropyl-2-(trifluoromethyl)benzimidazol-5-yl]-[4-(2-tetrahydropyran-4-yl-3H-imidazo[4,5-b]pyridin-7-yl)-1-piperidyl]methanone;
(2-amino-4-methylsulfonyl-phenyl)-[4-(2-tetrahydropyran-4-yl-3H-imidazo[4,5-b]pyridin-7-yl)-1-piperidyl]methanone;
[3-(1,1-dioxo-1,2-thiazolidin-2-yl)phenyl]-[4-(2-tetrahydropyran-4-yl-3H-imidazo[4,5-b]pyridin-7-yl)-1-piperidyl]methanone;
[2-(3-hydroxyphenyl)cyclopropyl]-[4-(2-tetrahydropyran-4-yl-3H-imidazo[4,5-b]pyridin-7-yl)-1-piperidyl]methanone;
2-[2-chloro-4-(trifluoromethyl)phenoxy]-1-[4-(2-tetrahydropyran-4-yl-3H-imidazo[4,5-b]pyridin-7-yl)-1-piperidyl]propan-1-one;
(3-phenylcyclopentyl)-[4-(2-tetrahydropyran-4-yl-3H-imidazo[4,5-b]pyridin-7-yl)-1-piperidyl]methanone;
[4-hydroxy-1-[2-(trifluoromethyl)phenyl]pyrazol-3-yl]-[4-(2-tetrahydropyran-4-yl-3H-imidazo[4,5-b]pyridin-7-yl)-1-piperidyl]methanone;
[4-[2-[5,5-difluoro-3-piperidyl]-3H-imidazo[4,5-b]pyridin-7-yl]-1-piperidyl]-[4-(trifluoromethoxy)phenyl]methanone;
[3-amino-4-phenyl-5-(trifluoromethyl)-2-thienyl]-[4-(2-tetrahydropyran-4-yl-3H-imidazo[4,5-b]pyridin-7-yl)-1-piperidyl]methanone;
(7-hydroxy-1H-indol-2-yl)-[4-(2-tetrahydropyran-4-yl-3H-imidazo[4,5-b]pyridin-7-yl)-1-piperidyl]methanone;
[4-[2-(1-cyclopropyl-4-piperidyl)-3H-imidazo[4,5-b]pyridin-7-yl]-1-piperidyl]-[4-(trifluoromethoxy)phenyl]methanone;
1-[4-[7-[1-[4-(trifluoromethoxy)benzoyl]-4-piperidyl]-3H-imidazo[4,5-b]pyridin-2-yl]-1-piperidyl]ethanone;
[2-amino-4-(pentafluoro-λ⁶-sulfanyl)phenyl]-[4-(2-tetrahydropyran-4-yl-3H-imidazo[4,5-b]pyridin-7-yl)-1-piperidyl]methanone;
(cis)-[4-(2-tetrahydropyran-4-yl-3H-imidazo[4,5-b]pyridin-7-yl)-1-piperidyl]-[4-(trifluoromethoxy)cyclohexyl]methanone;
1-[4-[7-[1-[2-amino-4-(trifluoromethoxy)benzoyl]-4-piperidyl]-3H-imidazo[4,5-b]pyridin-2-yl]-1-piperidyl]ethanone;
[2-amino-4-(trifluoromethoxy)phenyl]-[4-[2-(1-cyclopropyl-4-piperidyl)-3H-imidazo[4,5-b]pyridin-7-yl]-1-piperidyl]methanone;
[4-(2-cyclohexyl-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-(1,4-dioxan-2-yl)-3H-imidazo[4,5-b]pyridin-7-yl]-1-piperidyl]methanone;
2-[4-(2-tetrahydropyran-4-yl-3H-imidazo[4,5-b]pyridin-7-yl)piperidine-1-carbonyl]benzothiophene-5-carbonitrile;
(5-amino-1-phenyl-pyrazol-4-yl)-[4-(2-tetrahydropyran-4-yl-3H-imidazo[4,5-b]pyridin-7-yl)-1-piperidyl]methanone;
2-[2-oxo-2-[4-(2-tetrahydropyran-4-yl-3H-imidazo[4,5-b]pyridin-7-yl)-1-piperidyl]ethyl]-4H-1,4-benzoxazin-3-one;
1-(3,4-difluorophenyl)-4-[4-(2-tetrahydropyran-4-yl-3H-imidazo[4,5-b]pyridin-7-yl)piperidine-1-carbonyl]pyrrolidin-2-one;
[4-(3-chloro-4-fluoro-phenyl)phenyl]-[4-(2-tetrahydropyran-4-yl-3H-imidazo[4,5-b]pyridin-7-yl)-1-piperidyl]methanone;
(7-amino-2-methyl-pyrazolo[1,5-a]pyrimidin-6-yl)-[4-(2-tetrahydropyran-4-yl-3H-imidazo[4,5-b]pyridin-7-yl)-1-piperidyl]methanone;
1-[(2-chlorophenyl)methyl]-4-[4-(2-tetrahydropyran-4-yl-3H-imidazo[4,5-b]pyridin-7-yl)piperidine-1-carbonyl]pyrrolidin-2-one;
1-(4-fluorophenyl)-4-[4-(2-tetrahydropyran-4-yl-3H-imidazo[4,5-b]pyridin-7-yl)piperidine-1-carbonyl]pyrrolidin-2-one;
[1-[(2-chlorophenyl)methyl]pyrazol-4-yl]-[4-(2-tetrahydropyran-4-yl-3H-imidazo[4,5-b]pyridin-7-yl)-1-piperidyl]methanone;
[4-(2-aminophenyl)phenyl]-[4-(2-tetrahydropyran-4-yl-3H-imidazo[4,5-b]pyridin-7-yl)-1-piperidyl]methanone;
(3,6-dichloroimidazo[1,2-a]pyridin-2-yl)-[4-(2-tetrahydropyran-4-yl-3H-imidazo[4,5-b]pyridin-7-yl)-1-piperidyl]methanone;
(4-amino-1-ethyl-pyrazol-3-yl)-[4-(2-tetrahydropyran-4-yl-3H-imidazo[4,5-b]pyridin-7-yl)-1-piperidyl]methanone;
(3-aminoquinoxalin-2-yl)-[4-(2-tetrahydropyran-4-yl-3H-imidazo[4,5-b]pyridin-7-yl)-1-piperidyl]methanone;
2-[4-(2-tetrahydropyran-4-yl-3H-imidazo[4,5-b]pyridin-7-yl)piperidine-1-carbonyl]spiro[cyclopropane-1,3′-indoline]-2′-one;
[1-(2-chlorophenyl)pyrrolidin-3-yl]-[4-(2-tetrahydropyran-4-yl-3H-imidazo[4,5-b]pyridin-7-yl)-1-piperidyl]methanone;
(2-amino-4,5-dichloro-phenyl)-[4-(2-tetrahydropyran-4-yl-3H-imidazo[4,5-b]pyridin-7-yl)-1-piperidyl]methanone;
[1-(4-fluorophenyl)imidazol-4-yl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-(1-methoxy-1-methyl-ethyl)-3H-imidazo[4,5-b]pyridin-7-yl]-1-piperidyl]methanone;
[2-amino-4-(trifluoromethoxy)phenyl]-[4-[2-(1-hydroxy-1-methyl-ethyl)-3H-imidazo[4,5-b]pyridin-7-yl]-1-piperidyl]methanone;
[2-amino-4-(trifluoromethoxy)phenyl]-[4-(2-cyclohexyl-3H-imidazo[4,5-b]pyridin-7-yl)-1-piperidyl]methanone;
[2-amino-4-(trifluoromethoxy)phenyl]-[4-[2-(3-methoxypropyl)-3H-imidazo[4,5-b]pyridin-7-yl]-1-piperidyl]methanone;
[4-[2-(3-amino-1-bicyclo[1.1.1]pentanyl)-3H-imidazo[4,5-b]pyridin-7-yl]-1-piperidyl]-[4-(trifluoromethoxy)phenyl]methanone;
[4-[2-(3-amino-1-bicyclo[1.1.1]pentanyl)-3H-imidazo[4,5-b]pyridin-7-yl]-1-piperidyl]-[2-amino-4-(trifluoromethoxy)phenyl]methanone;
(trans)-[4-(2-tetrahydropyran-4-yl-3H-imidazo[4,5-b]pyridin-7-yl)-1-piperidyl]-[4-(trifluoromethoxy)cyclohexyl]methanone;
[3-fluoro-4-(trifluoromethoxy)phenyl]-[4-(2-tetrahydropyran-4-yl-3H-imidazo[4,5-b]pyridin-7-yl)-1-piperidyl]methanone;
(cis)-[2-amino-4-(trifluoromethoxy)phenyl]-[4-[2-(3-methoxycyclobutyl)-3H-imidazo[4,5-b]pyridin-7-yl]-1-piperidyl]methanone;
(trans)-[2-amino-4-(trifluoromethoxy)phenyl]-[4-[2-(3-methoxycyclobutyl)-3H-imidazo[4,5-b]pyridin-7-yl]-1-piperidyl]methanone;
[4-[2-[(4-methylpiperazin-1-yl)methyl]-3H-imidazo[4,5-b]pyridin-7-yl]-1-piperidyl]-[4-(trifluoromethoxy)phenyl]methanone;
[4-[2-[2-(5-chloro-2-hydroxy-phenyl)thiazol-4-yl]-3H-imidazo[4,5-b]pyridin-7-yl]-1-piperidyl]-[4-(trifluoromethoxy)phenyl]methanone;
[4-[2-(2-fluoro-5-hydroxy-phenyl)-3H-imidazo[4,5-b]pyridin-7-yl]-1-piperidyl]-[4-(trifluoromethoxy)phenyl]methanone;
N-[3-[7-[1-[4-(trifluoromethoxy)benzoyl]-4-piperidyl]-3H-imidazo[4,5-b]pyridin-2-yl]phenyl]tetrahydrofuran-2-carboxamide;
[4-[2-[5-(hydroxymethyl)isoxazol-3-yl]-3H-imidazo[4,5-b]pyridin-7-yl]-1-piperidyl]-[4-(trifluoromethoxy)phenyl]methanone;
1-ethyl-3-hydroxy-6-[7-[1-[4-(trifluoromethoxy)benzoyl]-4-piperidyl]-3H-imidazo[4,5-b]pyridin-2-yl]quinoxalin-2-one;
[3-(2-tetrahydropyran-4-yl-3H-imidazo[4,5-b]pyridin-7-yl)pyrrolidin-1-yl]-[4-(trifluoromethoxy)phenyl]methanone;
[2-amino-4-(trifluoromethoxy)phenyl]-[3-(2-tetrahydropyran-4-yl-3H-imidazo[4,5-b]pyridin-7-yl)pyrrolidin-1-yl]methanone;
(4-bromophenyl)-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[1,4-dioxan-2-yl]-3H-imidazo[4,5-b]pyridin-7-yl]-1-piperidyl]methanone;
4-[7-[1-[2-amino-4-(trifluoromethoxy)benzoyl]-4-piperidyl]-3H-imidazo[4,5-b]pyridin-2-yl]cyclohexanone;
[4-(2-tetrahydropyran-4-yl-3H-imidazo[4,5-b]pyridin-7-yl)-1-piperidyl]-(4-trimethylsilylphenyl)methanone;
(cis)-[4-[2-(3-hydroxycyclobutyl)-3H-imidazo[4,5-b]pyridin-7-yl]-1-piperidyl]-[4-(trifluoromethoxy)phenyl]methanone;
(trans)-[4-[2-(3-hydroxycyclobutyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-(3-methoxy-1-bicyclo[1.1.1]pentanyl)-3H-imidazo[4,5-b]pyridin-7-yl]-1-piperidyl]methanone;
(trans)-[4-(trifluoromethoxy)phenyl]-[4-[2-(4-methoxycyclohexyl)-3H-imidazo[4,5-b]pyridin-7-yl]-1-piperidyl]methanone;
(cis)-[4-(trifluoromethoxy)phenyl]-[4-[2-(4-methoxycyclohexyl)-3H-imidazo[4,5-b]pyridin-7-yl]-1-piperidyl]methanone;
(trans)-[2-amino-4-(trifluoromethoxy)phenyl]-[4-[2-(4-methoxycyclohexyl)-3H-imidazo[4,5-b]pyridin-7-yl]-1-piperidyl]methanone;
(cis)-[2-amino-4-(trifluoromethoxy)phenyl]-[4-[2-(4-methoxycyclohexyl)-3H-imidazo[4,5-b]pyridin-7-yl]-1-piperidyl]methanone;
(2,2-difluoro-1,3-benzodioxol-5-yl)-[4-(2-tetrahydropyran-4-yl-3H-imidazo[4,5-b]pyridin-7-yl)-1-piperidyl]methanone;
(4-bromo-1H-indol-7-yl)-[4-(2-tetrahydropyran-4-yl-3H-imidazo[4,5-b]pyridin-7-yl)-1-piperidyl]methanone;
[4-(2-tetrahydropyran-4-yl-3H-imidazo[4,5-b]pyridin-7-yl)-1-piperidyl]-[2-(trifluoromethyl)-1H-indol-6-yl]methanone;
[4-(cyclopropoxy)phenyl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[5,5-difluoro-3-piperidyl]-3H-imidazo[4,5-b]pyridin-7-yl]-1-piperidyl]methanone;
[2-amino-4-(trifluoromethoxy)phenyl]-[3-(2-tetrahydropyran-4-yl-3H-imidazo[4,5-b]pyridin-7-yl)pyrrolidin-1-yl]methanone;
2-tetrahydropyran-4-yl-7-[1-[4-(trifluoromethoxy)phenyl]sulfonyl-4-piperidyl]-3H-imidazo[4,5-b]pyridine;
[2-amino-4-(trifluoromethoxy)phenyl]-[4-[2-[oxepan-4-yl]-3H-imidazo[4,5-b]pyridin-7-yl]-1-piperidyl]methanone;
[4-[2-[rac-(2R,3S)-3-aminotetrahydrofuran-2-yl]-3H-imidazo[4,5-b]pyridin-7-yl]-1-piperidyl]-[4-(trifluoromethoxy)phenyl]methanone;
(cis)-[2-amino-4-(trifluoromethoxy)phenyl]-[4-[2-(3-hydroxycyclobutyl)-3H-imidazo[4,5-b]pyridin-7-yl]-1-piperidyl]methanone;
(trans)-[2-amino-4-(trifluoromethoxy)phenyl]-[4-[2-(3-hydroxycyclobutyl)-3H-imidazo[4,5-b]pyridin-7-yl]-1-piperidyl]methanone;
[3-(2-tetrahydropyran-4-yl-3H-imidazo[4,5-b]pyridin-7-yl)pyrrolidin-1-yl]-[4-(trifluoromethoxy)phenyl]methanone;
[2-amino-4-(trifluoromethoxy)phenyl]-[4-(2-morpholino-3H-imidazo[4,5-b]pyridin-7-yl)-1-piperidyl]methanone;
[2-amino-4-(trifluoromethoxy)phenyl]-[4-[2-(2-methylmorpholin-2-yl)-3H-imidazo[4,5-b]pyridin-7-yl]-1-piperidyl]methanone;
[4-[2-[6,6-dimethylmorpholin-2-yl]-3H-imidazo[4,5-b]pyridin-7-yl]-1-piperidyl]-[4-(trifluoromethoxy)phenyl]methanone;
[4-[2-[6,6-dimethylmorpholin-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[tetrahydrofuran-2-yl]-3H-imidazo[4,5-b]pyridin-7-yl]-1-piperidyl]methanone;
[4-[2-(7-oxa-4-azaspiro[2.5]octan-6-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[6,6-dimethylmorpholin-2-yl]-3H-imidazo[4,5-b]pyridin-7-yl]-1-piperidyl]methanone;
[2-amino-4-(trifluoromethoxy)phenyl]-[4-[2-[6,6-dimethylmorpholin-2-yl]-3H-imidazo[4,5-b]pyridin-7-yl]-1-piperidyl]methanone;
[4-[2-[1-(oxetan-3-yl)-4-piperidyl]-3H-imidazo[4,5-b]pyridin-7-yl]-1-piperidyl]-[4-(trifluoromethoxy)phenyl]methanone;
[4-[2-(2-oxabicyclo[2.2.2]octan-4-yl)-3H-imidazo[4,5-b]pyridin-7-yl]-1-piperidyl]-[4-(trifluoromethoxy)phenyl]methanone;
[4-(2-morpholino-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-[tetrahydrofuran-3-yl]-3H-imidazo[4,5-b]pyridin-7-yl]-1-piperidyl]methanone;
[2-amino-4-(trifluoromethoxy)phenyl]-[4-[2-[1-(oxetan-3-yl)-4-piperidyl]-3H-imidazo[4,5-b]pyridin-7-yl]-1-piperidyl]methanone;
[2-amino-3-fluoro-4-(trifluoromethoxy)phenyl]-[4-(2-tetrahydropyran-4-yl-3H-imidazo[4,5-b]pyridin-7-yl)-1-piperidyl]methanone;
[4-[2-[rac-(2R,3S)-3-aminotetrahydrofuran-2-yl]-3H-imidazo[4,5-b]pyridin-7-yl]-1-piperidyl]-[2-amino-4-(trifluoromethoxy)phenyl]methanone;
(cis)-[4-(2-tetrahydropyran-4-yl-3H-imidazo[4,5-b]pyridin-7-yl)cyclohexyl]-[4-(trifluoromethoxy)-1-piperidyl]methanone;
(trans)-[4-(2-tetrahydropyran-4-yl-3H-imidazo[4,5-b]pyridin-7-yl)cyclohexyl]-[4-(trifluoromethoxy)-1-piperidyl]methanone;
[4-[2-(4-amino-1-methyl-2-oxabicyclo[2.1.1]hexan-3-yl)-3H-imidazo[4,5-b]pyridin-7-yl]-1-piperidyl]-[2-amino-4-(trifluoromethoxy)phenyl]methanone;
(trans)-[4-[2-(3-methoxycyclobutyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-(7-oxa-4-azaspiro[2.5]octan-6-yl)-3H-imidazo[4,5-b]pyridin-7-yl]-1-piperidyl]methanone;
[2-amino-4-(trifluoromethoxy)phenyl]-[4-[2-(2-oxabicyclo[2.2.2]octan-4-yl)-3H-imidazo[4,5-b]pyridin-7-yl]-1-piperidyl]methanone;
[4-(2-cyclopent-3-en-1-yl-3H-imidazo[4,5-b]pyridin-7-yl)-1-piperidyl]-[4-(trifluoromethoxy)phenyl]methanone;
[2-amino-4-(trifluoromethoxy)phenyl]-[4-[2-[3-(trifluoromethyl)-1-bicyclo[1.1.1]pentanyl]-3H-imidazo[4,5-b]pyridin-7-yl]-1-piperidyl]methanone;
(2-amino-4-bromo-phenyl)-[4-(2-tetrahydropyran-4-yl-3H-imidazo[4,5-b]pyridin-7-yl)-1-piperidyl]methanone;
[4-[2-(4-amino-1-methyl-2-oxabicyclo[2.1.1]hexan-3-yl)-3H-imidazo[4,5-b]pyridin-7-yl]-1-piperidyl]-[4-(trifluoromethoxy)phenyl]methanone;
N-[2-[4-(2-tetrahydropyran-4-yl-3H-imidazo[4,5-b]pyridin-7-yl)piperidine-1-carbonyl]-5-(trifluoromethoxy)phenyl]acetamide;
(trans)-[4-[2-(3-methoxycyclobutyl)-3H-imidazo[4,5-b]pyridin-7-yl]-1-piperidyl]-[4-(trifluoromethoxy)phenyl]methanone;
[4-[2-(1,2,3,6-tetrahydropyridin-5-yl)-3H-imidazo[4,5-b]pyridin-7-yl]-1-piperidyl]-[4-(trifluoromethoxy)phenyl]methanone;
[4-[2-[4-phenylpyrrolidin-3-yl]-3H-imidazo[4,5-b]pyridin-7-yl]-1-piperidyl]-[4-(trifluoromethoxy)phenyl]methanone;
[4-[2-[4-fluoropyrrolidin-2-yl]-3H-imidazo[4,5-b]pyridin-7-yl]-1-piperidyl]-[4-(trifluoromethoxy)phenyl]methanone;
[4-[2-(2-amino-3,3,3-trifluoro-propyl)-3H-imidazo[4,5-b]pyridin-7-yl]-1-piperidyl]-[4-(trifluoromethoxy)phenyl]methanone;
[4-[2-[6-(aminomethyl)-3-pyridyl]-3H-imidazo[4,5-b]pyridin-7-yl]-1-piperidyl]-[4-(trifluoromethoxy)phenyl]methanone;
[4-[2-[rac-(3R,4S)-4-(4-pyridyl)pyrrolidin-3-yl]-3H-imidazo[4,5-b]pyridin-7-yl]-1-piperidyl]-[4-(trifluoromethoxy)phenyl]methanone;
[4-[2-(3-amino-2-bicyclo[2.2.1]hept-5-enyl)-3H-imidazo[4,5-b]pyridin-7-yl]-1-piperidyl]-[4-(trifluoromethoxy)phenyl]methanone;
[4-[2-[pyrrolidin-2-yl]-3H-imidazo[4,5-b]pyridin-7-yl]-1-piperidyl]-[4-(trifluoromethoxy)phenyl]methanone;
[4-[2-(3-amino-2-thienyl)-3H-imidazo[4,5-b]pyridin-7-yl]-1-piperidyl]-[4-(trifluoromethoxy)phenyl]methanone;
4-[7-[1-[4-(trifluoromethoxy)benzoyl]-4-piperidyl]-3H-imidazo[4,5-b]pyridin-2-yl]piperidine-4-carbonitrile;
[4-[2-[2-piperidyl]-3H-imidazo[4,5-b]pyridin-7-yl]-1-piperidyl]-[4-(trifluoromethoxy)phenyl]methanone;
[4-[2-(4-aminotetrahydropyran-4-yl)-3H-imidazo[4,5-b]pyridin-7-yl]-1-piperidyl]-[4-(trifluoromethoxy)phenyl]methanone;
[4-[2-(3-amino-4-methoxy-phenyl)-3H-imidazo[4,5-b]pyridin-7-yl]-1-piperidyl]-[4-(trifluoromethoxy)phenyl]methanone;
[4-[2-(2-piperidyl)-3H-imidazo[4,5-b]pyridin-7-yl]-1-piperidyl]-[4-(trifluoromethoxy)phenyl]methanone;
[4-[2-[pyrrolidin-2-yl]-3H-imidazo[4,5-b]pyridin-7-yl]-1-piperidyl]-[4-(trifluoromethoxy)phenyl]methanone;
[4-[2-(1,2,3,4-tetrahydroisoquinolin-6-yl)-3H-imidazo[4,5-b]pyridin-7-yl]-1-piperidyl]-[4-(trifluoromethoxy)phenyl]methanone;
[4-[2-(3-amino-5-hydroxy-phenyl)-3H-imidazo[4,5-b]pyridin-7-yl]-1-piperidyl]-[4-(trifluoromethoxy)phenyl]methanone;
[4-[2-(1H-indol-3-yl)-3H-imidazo[4,5-b]pyridin-7-yl]-1-piperidyl]-[4-(trifluoromethoxy)phenyl]methanone;
[4-[2-(4,5,6,7-tetrahydrothieno[3,2-c]pyridin-2-yl)-3H-imidazo[4,5-b]pyridin-7-yl]-1-piperidyl]-[4-(trifluoromethoxy)phenyl]methanone;
[4-[2-(azetidin-2-yl)-3H-imidazo[4,5-b]pyridin-7-yl]-1-piperidyl]-[4-(trifluoromethoxy)phenyl]methanone;2,2,2-trifluoroacetic acid
[4-[2-(3-oxa-9-azabicyclo[3.3.1]nonan-7-yl)-3H-imidazo[4,5-b]pyridin-7-yl]-1-piperidyl]-[4-(trifluoromethoxy)phenyl]methanone;
[4-[2-(4-amino-3-pyridyl)-3H-imidazo[4,5-b]pyridin-7-yl]-1-piperidyl]-[4-(trifluoromethoxy)phenyl]methanone;
[4-[2-[piperazin-2-yl]-3H-imidazo[4,5-b]pyridin-7-yl]-1-piperidyl]-[4-(trifluoromethoxy)phenyl]methanone;
[4-[2-(3-aminocyclobutyl)-3H-imidazo[4,5-b]pyridin-7-yl]-1-piperidyl]-[4-(trifluoromethoxy)phenyl]methanone;
[4-[2-[3-(methylamino)phenyl]-3H-imidazo[4,5-b]pyridin-7-yl]-1-piperidyl]-[4-(trifluoromethoxy)phenyl]methanone;
[4-[2-(2-azabicyclo[4.1.0]heptan-7-yl)-3H-imidazo[4,5-b]pyridin-7-yl]-1-piperidyl]-[4-(trifluoromethoxy)phenyl]methanone;
[4-[2-[4-hydroxy-6-(trifluoromethyl)-3-quinolyl]-3H-imidazo[4,5-b]pyridin-7-yl]-1-piperidyl]-[4-(trifluoromethoxy)phenyl]methanone;
[4-[2-(3-oxabicyclo[3.1.0]hexan-6-yl)-3H-imidazo[4,5-b]pyridin-7-yl]-1-piperidyl]-[4-(trifluoromethoxy)phenyl]methanone;
N-[4-hydroxy-2-[7-[1-[4-(trifluoromethoxy)benzoyl]-4-piperidyl]-3H-imidazo[4,5-b]pyridin-2-yl]phenyl]acetamide;
5,6-dimethyl-3-[7-[1-[4-(trifluoromethoxy)benzoyl]-4-piperidyl]-3H-imidazo[4,5-b]pyridin-2-yl]-1H-pyridin-2-one;
1-[3,5-dimethyl-4-[7-[1-[4-(trifluoromethoxy)benzoyl]-4-piperidyl]-3H-imidazo[4,5-b]pyridin-2-yl]-1H-pyrrol-2-yl]ethanone;
[4-[2-[1-(1,1-dioxothiolan-3-yl)-4-piperidyl]-3H-imidazo[4,5-b]pyridin-7-yl]-1-piperidyl]-[4-(trifluoromethoxy)phenyl]methanone;
[4-[2-(1-methylsulfonylazetidin-3-yl)-3H-imidazo[4,5-b]pyridin-7-yl]-1-piperidyl]-[4-(trifluoromethoxy)phenyl]methanone;
[2-hydroxy-4-(trifluoromethoxy)phenyl]-[4-(2-tetrahydropyran-4-yl-3H-imidazo[4,5-b]pyridin-7-yl)-1-piperidyl]methanone;
[4-[2-(1,4-oxazepan-2-yl)-3H-imidazo[4,5-b]pyridin-7-yl]-1-piperidyl]-[4-(trifluoromethoxy)phenyl]methanone;
[4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone;
[4-(1,1,2,2-tetrafluoroethoxy)phenyl]-[4-(2-tetrahydropyran-4-yl-3H-imidazo[4,5-b]pyridin-7-yl)-1-piperidyl]methanone;
[4-(1,1,2,2,2-pentafluoroethoxy)phenyl]-[4-(2-tetrahydropyran-4-yl-3H-imidazo[4,5-b]pyridin-7-yl)-1-piperidyl]methanone;
[4-(2-phenyl-3H-imidazo[4,5-b]pyridin-7-yl)-1-piperidyl]-[4-(trifluoromethoxy)phenyl]methanone;
(2,2,3,3-tetrafluoro-1,4-benzodioxin-6-yl)-[4-(2-tetrahydropyran-4-yl-3H-imidazo[4,5-b]pyridin-7-yl)-1-piperidyl]methanone;
[4-[2-[4-hydroxy-4-(trifluoromethyl)cyclohexyl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[7-oxa-4-azaspiro[2.5]octan-6-yl]-3H-imidazo[4,5-b]pyridin-7-yl]-1-piperidyl]methanone;
[4-[2-[rac-(1S,3S)-3-aminocyclohexyl]-3H-imidazo[4,5-b]pyridin-7-yl]-1-piperidyl]-[4-(trifluoromethoxy)phenyl]methanone;
[4-(2-cyclopentyl-3H-imidazo[4,5-b]pyridin-7-yl)-1-piperidyl]-[4-(trifluoromethoxy)phenyl]methanone;
[4-[2-(4-methylpiperazin-1-yl)-3H-imidazo[4,5-b]pyridin-7-yl]-1-piperidyl]-[4-(trifluoromethoxy)phenyl]methanone;
[4-[2-[7-oxa-4-azaspiro[2.5]octan-6-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-(2-cyclopent-3-en-1-yl-3H-imidazo[4,5-b]pyridin-7-yl)-1-piperidyl]methanone;
[4-[2-(azepan-3-yl)-3H-imidazo[4,5-b]pyridin-7-yl]-1-piperidyl]-[4-(trifluoromethoxy)phenyl]methanone;
[2-amino-4-(trifluoromethoxy)phenyl]-[4-(2-cyclopentyl-3H-imidazo[4,5-b]pyridin-7-yl)-1-piperidyl]methanone;
[4-[2-[4-amino-1-methyl-2-oxabicyclo[2.1.1]hexan-3-yl]-3H-imidazo[4,5-b]pyridin-7-yl]-1-piperidyl]-[2-amino-4-(trifluoromethoxy)phenyl]methanone;
[4-[2-(3-hydroxyphenyl)-3H-imidazo[4,5-b]pyridin-7-yl]-1-piperidyl]-[4-(trifluoromethoxy)phenyl]methanone;
[4-[2-[rac-(1R,3S)-3-aminocyclohexyl]-3H-imidazo[4,5-b]pyridin-7-yl]-1-piperidyl]-[4-(trifluoromethoxy)phenyl]methanone;
[4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[4-(2-tetrahydropyran-4-yl-3H-imidazo[4,5-b]pyridin-7-yl)azepan-1-yl]methanone;
[4-[2-(3-fluoro-4-piperidyl)-3H-imidazo[4,5-b]pyridin-7-yl]-1-piperidyl]-[4-(trifluoromethoxy)phenyl]methanone;
[4-[2-(1,2,3,6-tetrahydropyridin-4-yl)-3H-imidazo[4,5-b]pyridin-7-yl]-1-piperidyl]-[4-(trifluoromethoxy)phenyl]methanone;
3-[7-[1-[4-(trifluoromethoxy)benzoyl]-4-piperidyl]-3H-imidazo[4,5-b]pyridin-2-yl]bicyclo[1.1.1]pentane-1-carboxylic acid;
3-[4-(2-tetrahydropyran-4-yl-3H-imidazo[4,5-b]pyridin-7-yl)piperidine-1-carbonyl]-6-(trifluoromethyl)-1H-pyridin-2-one;
[4-(difluoromethoxy)phenyl]-[4-(2-tetrahydropyran-4-yl-3H-imidazo[4,5-b]pyridin-7-yl)-1-piperidyl]methanone;
[2-amino-4-(trifluoromethoxy)phenyl]-[4-[2-[1,1-dioxothiolan-3-yl]-3H-imidazo[4,5-b]pyridin-7-yl]-1-piperidyl]methanone;
[4-[2-[1,4-oxazepan-2-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[1,4-oxazepan-2-yl]-3H-imidazo[4,5-b]pyridin-7-yl]-1-piperidyl]methanone;
[4-(2-tetrahydropyran-4-yl-3H-imidazo[4,5-b]pyridin-7-yl)azepan-1-yl]-[4-(trifluoromethoxy)phenyl]methanone;
[2-amino-4-(trifluoromethoxy)phenyl]-[4-(2-tetrahydropyran-4-yl-3H-imidazo[4,5-b]pyridin-7-yl)azepan-1-yl]methanone;
(trans)-4-[7-[1-[4-(trifluoromethoxy)benzoyl]-4-piperidyl]-3H-imidazo[4,5-b]pyridin-2-yl]cyclohexanecarboxylic acid;
(cis)-[4-[2-[3-aminotetrahydrofuran-2-yl]-3H-imidazo[4,5-b]pyridin-7-yl]-1-piperidyl]-[2-amino-4-(trifluoromethoxy)phenyl]methanone;
[4-[2-(1,1-dioxothiolan-3-yl)-3H-imidazo[4,5-b]pyridin-7-yl]-1-piperidyl]-[4-(trifluoromethoxy)phenyl]methanone;
[4-[2-(4-hydroxy-4-methyl-cyclohexyl)-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(trifluoromethoxy)phenyl]-[4-[2-(4-hydroxy-4-methyl-cyclohexyl)-3H-imidazo[4,5-b]pyridin-7-yl]-1-piperidyl]methanone;
[2-amino-4-(trifluoromethoxy)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(trifluoromethoxy)phenyl]-[4-[2-(1,2,3,6-tetrahydropyridin-4-yl)-3H-imidazo[4,5-b]pyridin-7-yl]-1-piperidyl]methanone;
[4-[2-[azepan-3-yl]-3H-imidazo[4,5-b]pyridin-7-yl]-1-piperidyl]-[4-(trifluoromethoxy)phenyl]methanone;
[2-amino-4-(trifluoromethoxy)phenyl]-[4-[2-[azepan-3-yl]-3H-imidazo[4,5-b]pyridin-7-yl]-1-piperidyl]methanone;
[2-amino-4-(pentafluoro-λ⁶-sulfanyl)phenyl]-[4-(2-cyclopentyl-3H-imidazo[4,5-b]pyridin-7-yl)-1-piperidyl]methanone;
[4-(2-cyclopentyl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydrofuran-2-yl-3H-imidazo[4,5-b]pyridin-7-yl)-1-piperidyl]methanone;
(cis)-[4-[2-(4-methoxycyclohexyl)-3H-imidazo[4,5-b]pyridin-7-yl]-1-piperidyl]-[4-(pentafluoro-λ⁶-sulfanyl)phenyl]methanone;
(trans)-[4-[2-(4-methoxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
(cis)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone;
(trans)-[2-amino-4-(pentafluoro-λ⁶-sulfanyl)phenyl]-[4-[2-(4-methoxycyclohexyl)-3H-imidazo[4,5-b]pyridin-7-yl]-1-piperidyl]methanone;
[4-(pentafluoro-λ⁶-sulfanyl)phenyl]-[4-[2-[tetrahydrofuran-2-yl]-3H-imidazo[4,5-b]pyridin-7-yl]-1-piperidyl]methanone;
[4-(pentafluoro-λ⁶-sulfanyl)phenyl]-[4-[2-[tetrahydrofuran-3-yl]-3H-imidazo[4,5-b]pyridin-7-yl]-1-piperidyl]methanone;
[4-[2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[4-(pentafluoro-λ⁶-sulfanyl)phenyl]-[4-[2-(4-piperidyl)-3H-imidazo[4,5-b]pyridin-7-yl]-1-piperidyl]methanone;hydrochloride;
[4-[2-[morpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(3-methoxy-1-bicyclo[1.1.1]pentanyl)-3H-imidazo[4,5-b]pyridin-7-yl]-1-piperidyl]methanone;
[4-(2-N-morpholino-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-(4-piperidyl)-3H-imidazo[4,5-b]pyridin-7-yl]-1-piperidyl]methanone;
[2-amino-4-(pentafluoro-λ⁶-sulfanyl)phenyl]-[4-[2-[tetrahydrofuran-3-yl]-3H-imidazo[4,5-b]pyridin-7-yl]-1-piperidyl]methanone;
(cis)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone;
(trans)-[2-amino-4-(pentafluoro-λ⁶-sulfanyl)phenyl]-[4-[2-(3-methoxycyclobutyl)-3H-imidazo[4,5-b]pyridin-7-yl]-1-piperidyl]methanone;
[2-amino-4-(pentafluoro-λ⁶-sulfanyl)phenyl]-[4-(2-N-morpholino-3H-imidazo[4,5-b]pyridin-7-yl)-1-piperidyl]methanone;
(trans)-[4-[2-(3-methoxycyclobutyl)-3H-imidazo[4,5-b]pyridin-7-yl]-1-piperidyl]-[4-(pentafluoro-λ⁶-sulfanyl)phenyl]methanone;
[4-[2-(1-methyl-4-piperidyl)-3H-imidazo[4,5-b]pyridin-7-yl]-1-piperidyl]-[4-(pentafluoro-λ⁶-sulfanyl)phenyl]methanone;
[4-[2-(1-cyclopropyl-4-piperidyl)-3H-imidazo[4,5-b]pyridin-7-yl]-1-piperidyl]-[4-(pentafluoro-λ⁶-sulfanyl)phenyl]methanone;
[4-[2-[morpholin-2-yl]-3H-imidazo[4,5-b]pyridin-7-yl]-1-piperidyl]-[4-(pentafluoro-λ⁶-sulfanyl)phenyl]methanone;
(cis)-[4-[2-(3-methoxycyclobutyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(1-methyl-4-piperidyl)-3H-imidazo[4,5-b]pyridin-7-yl]-1-piperidyl]methanone;
[2-amino-4-(pentafluoro-λ⁶-sulfanyl)phenyl]-[4-[2-[morpholin-2-yl]-3H-imidazo[4,5-b]pyridin-7-yl]-1-piperidyl]methanone;
[2-amino-4-(pentafluoro-λ⁶-sulfanyl)phenyl]-[4-[2-(1-cyclopropyl-4-piperidyl)-3H-imidazo[4,5-b]pyridin-7-yl]-1-piperidyl]methanone;
[4-[2-(1-methoxy-1-methyl-ethyl)-3H-imidazo[4,5-b]pyridin-7-yl]-1-piperidyl]-[4-(pentafluoro-λ⁶-sulfanyl)phenyl]methanone;
[2-(methylamino)-4-(pentafluoro-λ⁶-sulfanyl)phenyl]-[4-[2-(1-methyl-4-piperidyl)-3H-imidazo[4,5-b]pyridin-7-yl]-1-piperidyl]methanone;
[2-amino-4-(pentafluoro-λ⁶-sulfanyl)phenyl]-[4-[2-(1-methoxy-1-methyl-ethyl)-3H-imidazo[4,5-b]pyridin-7-yl]-1-piperidyl]methanone;
(cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
[4-[2-[4-methylmorpholin-2-yl]-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
(cis)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[morpholin-2-yl]-3H-imidazo[4,5-b]pyridin-7-yl]-1-piperidyl]methanone;
(trans)-[4-[2-(4-hydroxycyclohexyl)-3H-imidazo[4,5-b]pyridin-7-yl]-1-piperidyl]-[4-(pentafluoro-λ⁶-sulfanyl)phenyl]methanone;
[4-[2-[4-methylmorpholin-2-yl]-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone;
[4-(pentafluoro-λ⁶-sulfanyl)phenyl]-[4-(2-tetrahydropyran-4-yl-3H-imidazo[4,5-b]pyridin-7-yl)azepan-1-yl]methanone;
[4-[2-[2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[2-(4-hydroxycyclohexyl)-3H-imidazo[4,5-b]pyridin-7-yl]-1-piperidyl]methanone;
3-methyl-3-[7-[1-[4-(pentafluoro-λ⁶-sulfanyl)benzoyl]-4-piperidyl]-3H-imidazo[4,5-b]pyridin-2-yl]cyclobutanone;
[4-[2-(1-hydroxy-1-methyl-ethyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(1-hydroxy-1-methyl-ethyl)-3H-imidazo[4,5-b]pyridin-7-yl]-1-piperidyl]methanone;
[(cis)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[(trans)-2-methyl-4-(2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[2-[2-oxa-5-azabicyclo[2.2.1]heptan-1-yl]-3H-imidazo[4,5-b]pyridin-7-yl]-1-piperidyl]methanone;
[2-amino-4-(trifluoromethoxy)phenyl]-[4-fluoro-4-(6-fluoro-2-tetrahydropyran-4-yl-3H-imidazo[4,5-b]pyridin-7-yl)-1-piperidyl]methanone;
[4-[2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-(3-hydroxy-1-bicyclo[1.1.1]pentanyl)-3H-imidazo[4,5-b]pyridin-7-yl]-1-piperidyl]methanone;
[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(pentafluoro-k-sulfanyl)phenyl]-[3-(2-tetrahydropyran-4-yl-3H-imidazo[4,5-b]pyridin-7-yl)pyrrolidin-1-yl]methanone;
[2-hydroxy-4-(pentafluoro-λ⁶-sulfanyl)phenyl]-[4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[4-(6-fluoro-2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(cis)-2-methyl-4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[(trans)-2-methyl-4-(2-tetrahydropyran-4-yl-3H-imidazo[4,5-b]pyridin-7-yl)-1-piperidyl]methanone;
(cis)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone;
(trans)-[2-amino-4-(pentafluoro-λ⁶-sulfanyl)phenyl]-[4-[6-fluoro-2-(4-methoxycyclohexyl)-3H-imidazo[4,5-b]pyridin-7-yl]-1-piperidyl]methanone;
[4-(6-fluoro-2-tetrahydropyran-4-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-(2-tetrahydropyran-4-yl-3H-imidazo[4,5-b]pyridin-7-yl)azepan-1-yl]methanone;
[2-amino-4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(pentafluoro-λ⁶-sulfanyl)phenyl]-[3-(2-tetrahydropyran-4-yl-3H-imidazo[4,5-b]pyridin-7-yl)pyrrolidin-1-yl]methanone;
[4-(6-fluoro-2-morpholin-2-yl-3H-imidazo[4,5-b]pyridin-7-yl)-1-piperidyl]-[4-(pentafluoro-λ⁶-sulfanyl)phenyl]methanone;
[4-[6-fluoro-2-(2-oxabicyclo[2.2.2]octan-4-yl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(2-oxabicyclo[2.2.2]octan-4-yl)-3H-imidazo[4,5-b]pyridin-7-yl]-1-piperidyl]methanone;
[4-(6-fluoro-2-tetrahydropyran-3-yl-3H-imidazo[4,5-b]pyridin-7-yl)-1-piperidyl]-[4-(pentafluoro-λ⁶-sulfanyl)phenyl]methanone;
(cis)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
(cis)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
[2-fluoro-4-(pentafluoro-λ⁶-sulfanyl)phenyl]-[4-(2-tetrahydropyran-4-yl-3H-imidazo[4,5-b]pyridin-7-yl)-1-piperidyl]methanone;
[2-amino-4-(pentafluoro-λ⁶-sulfanyl)phenyl]-[4-(6-fluoro-2-tetrahydropyran-3-yl-3H-imidazo[4,5-b]pyridin-7-yl)-1-piperidyl]methanone;
(trans)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone;
(cis)-[4-[6-fluoro-2-(4-methoxycyclohexyl)-3H-imidazo[4,5-b]pyridin-7-yl]-1-piperidyl]-[4-(pentafluoro-λ⁶-sulfanyl)phenyl]methanone;
[4-(6-fluoro-2-tetrahydrofuran-3-yl-3H-imidazo[4,5-b]pyridin-7-yl)-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(oxepan-4-yl)-3H-imidazo[4,5-b]pyridin-7-yl]-1-piperidyl]methanone;
[4-[6-fluoro-2-(oxepan-4-yl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(6-fluoro-2-tetrahydrofuran-3-yl-3H-imidazo[4,5-b]pyridin-7-yl)-1-piperidyl]methanone;
[4-[2-[6-methylmorpholin-2-yl]-3H-imidazo[4,5-b]pyridin-7-yl]-1-piperidyl]-[4-(pentafluoro-λ⁶-sulfanyl)phenyl]methanone;
[4-[6-fluoro-2-(1-methoxy-1-methyl-ethyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[2-[4-hydroxy-4-(trifluoromethyl)cyclohexyl]-3H-imidazo[4,5-b]pyridin-7-yl]-1-piperidyl]methanone;
[2-amino-4-(pentafluoro-λ⁶-sulfanyl)phenyl]-[4-[6-fluoro-2-(3-methoxy-1-bicyclo[1.1.1]pentanyl)-3H-imidazo[4,5-b]pyridin-7-yl]-1-piperidyl]methanone;
[4-[6-fluoro-2-(3-methoxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(6-fluoro-2-tetrahydrofuran-2-yl-3H-imidazo[4,5-b]pyridin-7-yl)-1-piperidyl]methanone;
[4-(6-fluoro-2-tetrahydrofuran-2-yl-3H-imidazo[4,5-b]pyridin-7-yl)-1-piperidyl]-[4-(pentafluoro-λ⁶-sulfanyl)phenyl]methanone;
[4-(6-fluoro-2-tetrahydropyran-4-yl-3H-imidazo[4,5-b]pyridin-7-yl)-1-piperidyl]-[2-hydroxy-4-(pentafluoro-λ⁶-sulfanyl)phenyl]methanone;
[2-amino-4-(pentafluoro-λ⁶-sulfanyl)phenyl]-[4-(6-fluoro-2-tetrahydropyran-2-yl-3H-imidazo[4,5-b]pyridin-7-yl)-1-piperidyl]methanone;
[2-amino-4-(pentafluoro-λ⁶-sulfanyl)phenyl]-[4-[2-[4-(hydroxymethyl)-1-piperidyl]-3H-imidazo[4,5-b]pyridin-7-yl]-1-piperidyl]methanone;
[4-[6-fluoro-2-(3-methoxycyclobutyl)-3H-imidazo[4,5-b]pyridin-7-yl]-1-piperidyl]-[4-(pentafluoro-λ⁶-sulfanyl)phenyl]methanone;
[4-(6-fluoro-2-tetrahydropyran-2-yl-3H-imidazo[4,5-b]pyridin-7-yl)-1-piperidyl]-[4-(pentafluoro-λ⁶-sulfanyl)phenyl]methanone;
(trans)-[4-[6-fluoro-2-(4-hydroxycyclohexyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
[4-[6-fluoro-2-[2-oxa-5-azabicyclo[2.2.1]heptan-1-yl]-3H-imidazo[4,5-b]pyridin-7-yl]-1-piperidyl]-[4-(pentafluoro-λ⁶-sulfanyl)phenyl]methanone;
[4-[2-[3-methoxycyclohexyl]-3H-imidazo[4,5-b]pyridin-7-yl]-1-piperidyl]-[4-(pentafluoro-λ⁶-sulfanyl)phenyl]methanone;
[4-(6-fluoro-2-thiomorpholin-2-yl-3H-imidazo[4,5-b]pyridin-7-yl)-1-piperidyl]-[4-(pentafluoro-λ⁶-sulfanyl)phenyl]methanone;
(trans)-[4-[6-fluoro-2-(4-methoxycyclohexyl)-3H-imidazo[4,5-b]pyridin-7-yl]-1-piperidyl]-[2-hydroxy-4-(pentafluoro-λ⁶-sulfanyl)phenyl]methanone;
[2-amino-4-(pentafluoro-λ⁶-sulfanyl)phenyl]-[4-[6-fluoro-2-(1-methoxy-1-methyl-ethyl)-3H-imidazo[4,5-b]pyridin-7-yl]-1-piperidyl]methanone;
[4-[6-fluoro-2-[tetrahydrofuran-3-yl]-3H-imidazo[4,5-b]pyridin-7-yl]-1-piperidyl]-[4-(pentafluoro-λ⁶-sulfanyl)phenyl]methanone;
[4-[6-fluoro-2-[6-methylmorpholin-2-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[tetrahydrofuran-3-yl]-3H-imidazo[4,5-b]pyridin-7-yl]-1-piperidyl]methanone;
[4-[6-fluoro-2-[morpholin-2-yl]-3H-imidazo[4,5-b]pyridin-7-yl]-1-piperidyl]-[4-(pentafluoro-λ⁶-sulfanyl)phenyl]methanone;
5-[6-fluoro-7-[1-[4-(pentafluoro-λ⁶-sulfanyl)benzoyl]-4-piperidyl]-3H-imidazo[4,5-b]pyridin-2-yl]piperidin-2-one;
(trans)-[4-[2-(3-hydroxycyclobutyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-(2-cyclopentyl-6-fluoro-3H-imidazo[4,5-b]pyridin-7-yl)-1-piperidyl]methanone;
[2-amino-4-(pentafluoro-λ⁶-sulfanyl)phenyl]-[4-[2-[3-methoxycyclohexyl]-3H-imidazo[4,5-b]pyridin-7-yl]-1-piperidyl]methanone;
[4-[6-fluoro-2-[oxepan-4-yl]-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-[oxepan-4-yl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[tetrahydrofuran-2-yl]-3H-imidazo[4,5-b]pyridin-7-yl]-1-piperidyl]methanone;
[4-[6-fluoro-2-[tetrahydrofuran-2-yl]-3H-imidazo[4,5-b]pyridin-7-yl]-1-piperidyl]-[4-(pentafluoro-λ⁶-sulfanyl)phenyl]methanone;
[4-[6-fluoro-2-(3-hydroxy-1-bicyclo[1.1.1]pentanyl)-3H-imidazo[4,5-b]pyridin-7-yl]-1-piperidyl]-[4-(pentafluoro-λ⁶-sulfanyl)phenyl]methanone;
[2-amino-4-(pentafluoro-λ⁶-sulfanyl)phenyl]-[4-[6-fluoro-2-(3-hydroxy-1-bicyclo[1.1.1]pentanyl)-3H-imidazo[4,5-b]pyridin-7-yl]-1-piperidyl]methanone;
(trans)-[2-amino-4-(pentafluoro-λ⁶-sulfanyl)phenyl]-[4-[2-(3-hydroxycyclobutyl)-3H-imidazo[4,5-b]pyridin-7-yl]-1-piperidyl]methanone;
[4-(2-cyclopentyl-6-fluoro-3H-imidazo[4,5-b]pyridin-7-yl)-1-piperidyl]-[4-(pentafluoro-λ⁶-sulfanyl)phenyl]methanone;
[4-[6-fluoro-2-(3-methoxy-1-bicyclo[1.1.1]pentanyl)-3H-imidazo[4,5-b]pyridin-7-yl]-1-piperidyl]-[4-(trifluoromethoxy)phenyl]methanone;
[4-[6-fluoro-2-(3-methoxy-1-bicyclo[1.1.1]pentanyl)-3H-imidazo[4,5-b]pyridin-7-yl]-1-piperidyl]-[2-hydroxy-4-(pentafluoro-λ⁶-sulfanyl)phenyl]methanone;
[2-amino-4-(trifluoromethoxy)phenyl]-[4-[6-fluoro-2-(3-methoxy-1-bicyclo[1.1.1]pentanyl)-3H-imidazo[4,5-b]pyridin-7-yl]-1-piperidyl]methanone;
[4-[6-fluoro-2-(2-oxabicyclo[2.2.2]octan-4-yl)-3H-imidazo[4,5-b]pyridin-7-yl]-1-piperidyl]-[4-(trifluoromethoxy)phenyl]methanone;
[2-amino-5-(pentafluoro-λ⁶-sulfanyl)phenyl]-[4-(2-tetrahydropyran-4-yl-3H-imidazo[4,5-b]pyridin-7-yl)-1-piperidyl]methanone;
[4-[2-(1-cyclopropyl-4-piperidyl)-6-fluoro-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-[3-(trifluoromethyl)-1-bicyclo[1.1.1]pentanyl]-3H-imidazo[4,5-b]pyridin-7-yl]-1-piperidyl]methanone;
[2-amino-4-(pentafluoro-λ⁶-sulfanyl)phenyl]-[4-[6-fluoro-2-[tetrahydropyran-2-yl]-3H-imidazo[4,5-b]pyridin-7-yl]-1-piperidyl]methanone;
[4-(6-fluoro-2-morpholin-2-yl-3H-imidazo[4,5-b]pyridin-7-yl)-1-piperidyl]-[2-hydroxy-4-(pentafluoro-λ⁶-sulfanyl)phenyl]methanone;
[4-[6-fluoro-2-[3-(trifluoromethyl)-1-bicyclo[1.1.1]pentanyl]-3H-imidazo[4,5-b]pyridin-7-yl]-1-piperidyl]-[4-(trifluoromethoxy)phenyl]methanone;
[4-[2-(1-methoxy-1-methyl-ethyl)-3H-imidazo[4,5-b]pyridin-7-yl]-1-piperidyl]-[4-(trifluoromethoxy)phenyl]methanone;
[4-[6-fluoro-2-[tetrahydropyran-2-yl]-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone;
(cis)-[2-amino-4-(pentafluoro-λ⁶-sulfanyl)phenyl]-[4-[6-fluoro-2-(4-hydroxycyclohexyl)-3H-imidazo[4,5-b]pyridin-7-yl]-1-piperidyl]methanone;
(cis)-[4-[6-fluoro-2-(4-methoxycyclohexyl)-3H-imidazo[4,5-b]pyridin-7-yl]-1-piperidyl]-[4-(trifluoromethoxy)phenyl]methanone;
(trans)-[4-[6-fluoro-2-(4-methoxycyclohexyl)-3H-imidazo[4,5-b]pyridin-7-yl]-1-piperidyl]-[4-(trifluoromethoxy)phenyl]methanone;
[4-[6-fluoro-2-(1-methoxy-1-methyl-ethyl)-3H-imidazo[4,5-b]pyridin-7-yl]-1-piperidyl]-[4-(trifluoromethoxy)phenyl]methanone;
(trans)-(4-(6-fluoro-2-(4-hydroxy-4-methylcyclohexyl)-3H-imidazo[4,5-b]pyridin-7-yl)piperidin-1-yl)(4-(pentafluoro-λ⁶-sulfanyl)phenyl)methanone;
(cis)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-(1-methoxy-1-methyl-ethyl)-3H-imidazo[4,5-b]pyridin-7-yl]-1-piperidyl]methanone;
(trans)-[4-[6-fluoro-2-(3-hydroxycyclobutyl)-3H-imidazo[4,5-b]pyridin-7-yl]-1-piperidyl]-[4-(pentafluoro-λ⁶-sulfanyl)phenyl]methanone;
4-(6-fluoro-2-N-morpholino-3H-imidazo[4,5-b]pyridin-7-yl)-1-piperidyl]-[4-(pentafluoro-λ⁶-sulfanyl)phenyl]methanone;
(trans)-[4-[6-fluoro-2-[4-hydroxy-4-(trifluoromethyl)cyclohexyl]-3H-imidazo[4,5-b]pyridin-7-yl]-1-piperidyl]-[4-(pentafluoro-λ⁶-sulfanyl)phenyl]methanone;
[2-amino-4-(trifluoromethoxy)phenyl]-[4-[6-fluoro-2-(2-oxabicyclo[2.2.2]octan-4-yl)-3H-imidazo[4,5-b]pyridin-7-yl]-1-piperidyl]methanone;
[4-[2-(3,4,4a,5,7,7a-hexahydro-2H-furo[3,4-b][1,4]oxazin-2-yl)-6-fluoro-3H-imidazo[4,5-b]pyridin-7-yl]-1-piperidyl]-[4-(pentafluoro-λ⁶-sulfanyl)phenyl]methanone;
(trans)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
(cis)-[2-amino-4-(pentafluoro-λ⁶-sulfanyl)phenyl]-[4-[6-fluoro-2-(3-methoxycyclobutyl)-3H-imidazo[4,5-b]pyridin-7-yl]-1-piperidyl]methanone;
2-[6-fluoro-7-[1-[4-(pentafluoro-λ⁶-sulfanyl)benzoyl]-4-piperidyl]-3H-imidazo[4,5-b]pyridin-2-yl]-2,3,4a,5,7,7a-hexahydrofuro[3,4-b][1,4]oxazine-4-carboxylic acid;
[4-[2-(1,1-dioxo-1,4-thiazinan-2-yl)-6-fluoro-3H-imidazo[4,5-b]pyridin-7-yl]-1-piperidyl]-[4-(pentafluoro-λ⁶-sulfanyl)phenyl]methanone;
[4-[6-fluoro-2-(3-methoxycyclobutyl)-3H-imidazo[4,5-b]pyridin-7-yl]-1-piperidyl]-[4-(trifluoromethoxy)phenyl]methanone;
(2-amino-4-(trifluoromethoxy) phenyl]-[4-[6-fluoro-2-(3-methoxycyclobutyl)-3H-imidazo[4,5-b]pyridin-7-yl]-1-piperidyl]methanone;
(cis)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone;
(trans)-[2-amino-4-(trifluoromethoxy)phenyl]-[4-[6-fluoro-2-(4-methoxycyclohexyl)-3H-imidazo[4,5-b]pyridin-7-yl]-1-piperidyl]methanone; and
7-[6-fluoro-7-[1-[4-(trifluoromethoxy)benzoyl]-4-piperidyl]-3H-imidazo[4,5-b]pyridin-2-yl]-2-oxa-5-azaspiro[3.5]nonane-5-carboxylic acid, and the pharmaceutically acceptable salts thereof.

24. The compound of any one of the preceding claims, wherein the compound has an IC₅₀ value of less than about 2 μM, e.g., less than about 1 μM, 0.5 μM, 0.2 μM, 100 nM, or 50 nM, against ERK5.

25. A pharmaceutical composition comprising the compound of any one of claims 1-24 and at least one pharmaceutically acceptable excipient or carrier.

26. A compound according to any one of claims 1-24, or a pharmaceutical composition according to claim 25, for use in therapy.

27. A compound according to any one of claims 1-24, or a pharmaceutical composition according to claim 25, for use in the treatment or prevention of cancer.

28. The compound or pharmaceutical composition for use according to claim 27, wherein the cancer is characterized by increased MAPK7 expression and/or increased ERK5 activity.

29. The compound or pharmaceutical composition for use according to claim 27 or claim 28, wherein the cancer is selected from leukaemia, breast cancer, multiple myeloma, colon cancer, renal cell carcinoma, mesothelioma, adenocarcinoma, neuroblastoma, and hepatocellular carcinoma.
